(19) United States
(12) Patent Application Publication
(10) Pub. No.: US 2006/0257987 A1
(43) Pub. Date: Nov. 16, 2006

(54) Ppar modulators

(76) Inventors: Isabel Cristina Gonzalez Valcarcel, Indianapolis, IN (US); Nathan Bryan Mantlo, Brownsburg, IN (US); Qing Shi, Carmel, IN (US); Minmin Wang, Fishers, IN (US); Leonard Larry Winneroski JR., Greenwood, IN (US); Yanping Xu, Noblesville, IN (US); Jeremy Schulenburg York, Noblesville, IN (US)

Correspondence Address: ELI LILLY & COMPANY, PATENT DIVISION, P.O. BOX 6288, INDIANAPOLIS, IN, 46206-6288, US

(21) Appl. No.: 10/566,291

(22) PCT Filed: Aug. 17, 2004
(86) PCT No.: PCT/US04/24381

§ 371 Date: Jan. 25, 2006

Related U.S. Application Data
(60) Provisional application No. 60/496,549, filed on Aug. 20, 2003.

(51) Int. Cl. C12N 9/96

(52) U.S. Cl. 435/188

(57) ABSTRACT

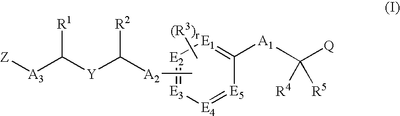

The present invention is directed to a compound of formula I, or a pharmaceutically acceptable salt, solvate, hydrate or stereoisomer thereof, which is useful in treating or preventing disorders mediated by a peroxisome proliferator activated receptor (PPAR) such as syndrome X, type II diabetes, hyperglycemia, hyperlipidemia, obesity, coagaulopathy, hypertension, arteriosclerosis, and other disorders related to syndrome X and cardiovascular diseases.

### FIELD OF THE INVENTION

The present invention relates to compounds of peroxisome proliferator activated receptor (PPAR) agonists, more specifically compounds of PPAR gamma-delta dual agonists, which are useful for the treatment and/or prevention of disorders modulated by a PPAR agonist.

### BACKGROUND OF THE INVENTION

The peroxisome proliferator activated receptors (PPARs) are members of the nuclear receptor gene family that are activated by fatty acids and fatty acid metabolites. The PPARs belong to the subset of nuclear receptors that function as heterodimers with the 9-cis retinoic acid receptor (RXR). Three subtypes, designated PPARα, PPARγ and PPARδ, are found in species ranging from Xenopus to humans.

PPARα is the main subtype in the liver and has facilitated analysis of the mechanism by which peroxisome proliferators exert their pleiotropic effects. PPARα is activated by a number of medium and long-chain fatty acids, and it is involved in stimulating β-oxidation of fatty acids. PPARα is also involved with the activity of fibrates and fatty acids in rodents and humans. Fibric acid derivatives such as clofibrate, fenofibrate, bezafibrate, ciprofibrate, beclofibrate and etofibrate, as well as gemfibrozil, produce a substantial reduction in plasma triglycerides along with moderate reduction in low-density lipoprotein (LDL) cholesterol, and they are used particularly for the treatment of hypertriglyceridemia.

PPARγ is the main subtype in adipose tissue and involved in activating the program of adipocyte differentiation. PPARγ is not involved in stimulating peroxisome proliferation in the liver. There are two isomers of PPARγ: PPARγ1 and PPARγ2, which differ only in that PPARγ2 contains an additional 28 amino acids present at the amino terminus. The DNA sequences for the PPARγ receptors are described in Elbrecht, et al., BBRC 224:431-437 (1996). Although peroxisome proliferators, including the fibrates and fatty acids, activate the transcriptional activity of PPAR's, only prostaglandin J₂ derivatives have been identified as natural ligands for PPARγ, which also binds the anti-diabetic agents thiazolidinediones with high affinity. The physiological functions of PPARα and PPARγ in lipid and carbohydrate metabolism were uncovered once it was recognized that they were the receptors for the fibrate and glitazone drugs, respectively.

PPARα and PPARγ receptors have been implicated in diabetes mellitus, cardiovascular disease, obesity, and gastrointestinal disease, such as inflammatory bowel disease and other inflammation related illnesses. Such inflammation related illnesses include, but are not limited to Alzheimer's disease, Crohn's disease, rheumatoid arthritis, psoriasis, and ischemia reprofusion injury.

By contrast, PPARδ (also referred to as PPARβ and NUC1) is not reported to be receptor for any known class of drug molecules, and its role in mammalian physiology has remained undefined. The human nuclear receptor gene PPARδ (hPPARδ) has been cloned from a human osteosarcoma cell cDNA library and is fully described in A. Schmidt et al., Molecular Endocrinology, 6:1634-1641 (1992).

Diabetes is a disease in which a mammal's ability to regulate glucose levels in the blood is impaired because the mammal has a reduced ability to convert glucose to glycogen for storage in muscle and liver cells. In Type I diabetes, this reduced ability to store glucose is caused by reduced insulin production. “Type II Diabetes” or “non-insulin dependent diabetes mellitus” (NIDDM) is the form of diabetes, which is due to a profound resistance to insulin stimulating or regulatory effect on glucose and lipid metabolism in the main insulin-sensitive tissues, muscle, liver and adipose tissue. This resistance to insulin responsiveness results in insufficient insulin activation of glucose uptake, oxidation and storage in muscle and inadequate insulin repression of lipolysis in adipose tissue and of glucose production and secretion in liver. When these cells become desensitized to insulin, the body tries to compensate by producing abnormally high levels of insulin and hyperinsulemia results. Hyperinsulemia is associated with hypertension and elevated body weight. Since insulin is involved in promoting the cellular uptake of glucose, amino acids and triglycerides from the blood by insulin sensitive cells, insulin insensitivity can result in elevated levels of triglycerides and LDL (known as the “bad” cholesterol) which are risk factors in cardiovascular diseases. The constellation of symptoms which includes hyperinsulemia combined with hypertension, elevated body weight, elevated triglycerides and elevated LDL is known as Syndrome X.

Hyperlipidemia is a condition which is characterized by an abnormal increase in serum lipids, such as cholesterol, triglycerides and phospholipids. These lipids do not circulate freely in solution in plasma, but are bound to proteins and transported as macromolecular complexes called lipoproteins. One form of hyperlipidemia is hypercholesterolemia, characterized by the existence of elevated LDL cholesterol levels. The initial treatment for hypercholesterolemia is often a diet low in fat and cholesterol coupled with appropriate physical exercise. Drug intervention is initiated if LDL-lowering goals are not met by diet and exercise alone. It is desirable to lower elevated levels of LDL cholesterol and increase levels of HDL cholesterol. Generally, it has been found that increased levels of HDL are associated with lower risk for coronary heart disease (CHD). See Gordon, et al., Am. J. Med., 62, 707-714 (1977); Stampfer, et al., N. England J. Med., 325, 373-381 (1991); and Kannel, et al., Ann. Internal Med., 90, 85-91 (1979). An example of an HDL raising agent is nicotinic acid, but the quantities needed to achieve HDL elevation are associated with undesirable effects, such as flushing.

There are several treatments currently available for treating diabetes mellitus but these treatments still remain unsatisfactory and have limitations. While physical exercise and reduction in dietary intake of calories will improve the diabetic condition, compliance with this approach can be poor because of sedentary lifestyles and excess food consumption, in particular high fat-containing food. Therefore, treatment with hypoglycemics, such as sulfonylureas (e.g., chlorpropamide, tolbutamide, tolazamide and acetohexamide) and biguanides (e.g. phenformin and metformin) are often necessary as the disease progresses. Sulfonylureas stimulate the β cells of the pancreas to secrete more insulin as the disease progresses. However, the response of the β cells eventually fails and treatment with insulin injections is necessary. In addition, both sulfonylurea treatment and insulin injection have the life threatening side effect of hypoglycemic coma, and thus patients using these treatments must carefully control dosage.

It has been well established that improved glycemic control in patients with diabetes (Type I and Type II) is accompanied by decreased microvasclular complications (DCCT and UKPDS). Due to difficulty in maintaining adequate glycemic control over time in patients with Type II diabetes, the use of insulin sensitizers in the therapy of Type II diabetes is growing. There is also a growing body of evidence that PPARγ agonist, insulin sensitizer, may have benefits in the treatment of Type II diabetes beyond their effects in improving glycemic control.

In the last decade a class of compounds known as thiazolidinediones (TZD) (e.g. U.S. Pat. Nos. 5,089,514; 4,342,771; 4,367,234; 4,340,605; and 5,306,726) have emerged as effective antidiabetic agents that have been shown to increase the sensitivity of insulin sensitive tissues, such as skeletal muscle, liver and adipose, to insulin. Increasing insulin sensitivity rather than the amount of insulin in the blood reduces the likelihood of hypoglycemic coma. Although thiazolidinediones have been shown to increase insulin sensitivity by binding to PPARγ receptors, this treatment also produces unwanted side effects such as weight gain and edema and, for troglitazone, liver toxicity. Recently, the compounds that are not TZDs have also been reported as PPAR modulators.

Adams et al. (WO 97/28115, WO 97/28135 and U.S. Pat. No. 5,895,051) discloses acetylphenols, which are useful as antiobesity and antidiabetic compounds.

Leibowitz et al. (WO 97/28149) discloses compounds which are PPARδ agonists and useful for treating cardiovascular diseases and related conditions.

Brooks et al. (WO 02/100813) discloses compounds of PPAR modulators that are useful for treating type II diabetes and other PPAR-mediated diseases and conditions.

In view of the above, an objective of the present invention is to provide new pharmaceutical agents which modulate PPAR receptors to prevent, treat and/or alleviate these diseases or conditions while reducing and or eliminating one or more of the unwanted side effects associated with the current treatments.

### SUMMARY OF THE INVENTION

The present invention relates to a compound of novel peroxisome proliferator activated receptor (PPAR) agonist having a structural formula I, 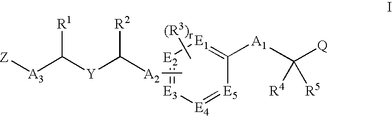
or a pharmaceutically acceptable salt, solvate, hydrate or stereoisomer thereof, wherein:
A₁ is: a bond, CH₂, O or S, and wherein A₁ and R⁴ or A₁ and R⁵ together being a 3- to 6-membered carbocyclyl when A₁ is a carbon;
A₂ and A₃ are independently: CH₂, O or S;
E₁, E₂, E₃, E₄ and E₅ are each CH or substituted carbon bearing A₂ and R³; or at least one of E₁, E₂, E₃, E₄ and E₅ is nitrogen and each of others being CH or substituted carbon bearing A₂ and R³;
Q is: —C(O)OR⁶, or R^{6A};
Y is: a bond, C₁-C₆ alkyl or C₃-C₆ cycloalkyl;
Z is: a) aryl;
   b) a 5- to 10-membered heteroaryl wherein the heteroaryl containing at least one heteroatom selected from N, O or S,
   c) bi-aryl, wherein biaryl being defined as aryl substituted with another aryl or aryl substituted with heteroaryl, or
   d) bi-heteroaryl, wherein bi-heteroaryl being defined as heteroaryl substituted with another heteroaryl, or heteroaryl substituted with aryl, and wherein aryl, heteroaryl, bi-aryl and bi-heteroaryl being optionally substituted with one or more groups independently selected from R⁷;
n is: 1, 2, 3, 4, 5 or 6
p is: 1 or 2;
r is: 1, 2, 3, or 4;
R¹ and R² are each independently:
   hydrogen,
   haloalkyl,
   C₁-C₆ alkyl,
   (CH₂)ₙC₃-C₈ cycloalkyl, or
   R¹ and R² form a 4- to 8-membered nonaromatic carbocyclic ring; and wherein at least one of R¹ and R² is alkyl or cycloalkyl, and;
R³ is: hydrogen,
   nitro,
   cyano,
   hydroxyl,
   halo,
   haloalkyl,
   haloalkyloxy,
   aryloxy,
   C₁-C₆ alkyl,
   C₁-C₆ alkoxy or
   C₃-C₈ cycloalkyl;
R⁴ and R⁵ are each independently: hydrogen or C₁-C₆ alkyl;
R⁶ is: hydrogen, C₁-C₆ alkyl or aminoalkyl;
R^{6A} is: carboxamide, sulfonamide, acylsulfonamide, tetrazole, 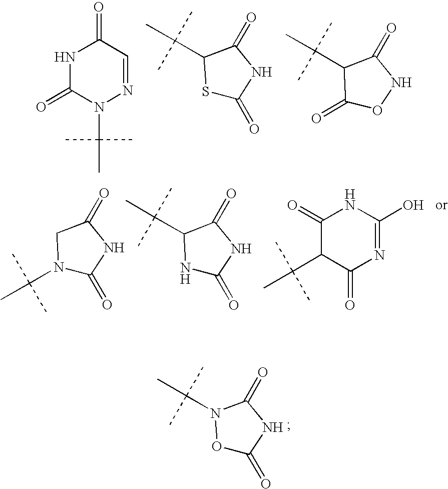
R⁷ is: hydrogen,
   oxo,
   nitro,
   cyano,
   hydroxyl,
   halo,
   haloalkyl,
   haloalkyloxy,
   aryloxy,
   arylalkyl,
   aminoalkyl,
   C₁-C₆ alkyl
   C₁-C₆ alkoxy,
   (CH₂)ₙC₃-C₈ cycloalkyl,
   C(O)R⁹,
   C(O)OR⁹,
   C(═NOR⁸)R⁹,
   CR⁸(OH)R⁹,
   C[═C(R⁸)₂]R⁹,
   OR⁹,
   SR⁹ or
   S(O)ₚR⁹;
R⁸ is: hydrogen or C₁-C₆ alkyl; and
R⁹ is: hydrogen,
   C₁-C₆ alkyl,
   C₃-C₈ cycloalkyl,
   aryl,
   heteroaryl or
   heterocyclyl,
   wherein alkyl, cycloalkyl, aryl, heteroaryl or heterocyclyl being optionally substituted with one or more substituents selected from the group consisting of:
   hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

The compounds of the present invention are useful in the treatment and/or prevention of diseases or condition relates to hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component.

In one embodiment, the present invention also relates to a pharmaceutical composition which comprising a compound of the present invention, or a pharmaceutically acceptable salt, solvate, hydrate or stereoisomer thereof and a pharmaceutically acceptable carrier. Within the scope of this invention also include a pharmaceutical composition containing additional therapeutic agent as well a compound of the present invention, or a pharmaceutically acceptable salt, solvate, hydrate or stereoisomer thereof and a pharmaceutically acceptable carrier.

In another embodiment, the present invention relates to a method of modulating a PPAR by contacting the receptor with a compound of the present invention, or a pharmaceutically acceptable salt, solvate and hydrate or stereoisomer thereof.

### DETAILED DESCRIPTION OF THE INVENTION

The compounds of the present invention are directed to peroxisome proliferator activated receptor (PPAR) agonists, more specifically compounds of PPARγ/δ dual agonists, which are useful for the treatment and/or prevention of disorders modulated by a PPAR, such as Type II diabetes, hyperglycemia, dyslipidemia, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other related diseases.

An embodiment of the present invention is a compound of novel peroxisome proliferator activated receptor (PPAR) agonists having a structural formula I, 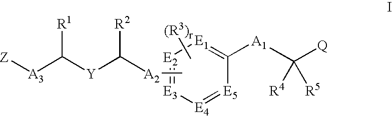
or a pharmaceutically acceptable salt, solvate, hydrate or stereoisomer thereof, wherein:
A₁ is: a bond, CH₂, O or S, and wherein A₁ and R⁴ or A₁ and R⁵ together being a 3- to 6-membered carbocyclyl when A₁ is a carbon;
A₂ and A₃ are independently: CH₂, O or S;
E₁, E₂, E₃, E₄ and E₅ are each CH or substituted carbon bearing A₂ and R³; or at least one of E₁, E₂, E₃, E₄ and E₅ is nitrogen and each of others being CH or substituted carbon bearing A₂ and R³;
Q is: —C(O)OR⁶, or R^{6A};
Y is: a bond, C₁-C₆ alkyl or C₃-C₆ cycloalkyl;
Z is: a) aryl;
   b) a 5- to 10-membered heteroaryl wherein the heteroaryl containing at least one heteroatom selected from N, O or S,
   c) bi-aryl, wherein biaryl being defined as aryl substituted with another aryl or aryl substituted with heteroaryl, or
   d) bi-heteroaryl, wherein bi-heteroaryl being defined as heteroaryl substituted with another heteroaryl, or heteroaryl substituted with aryl, and wherein aryl, heteroaryl, bi-aryl and bi-heteroaryl being optionally substituted with one or more groups independently selected from R⁷;
n is: 1, 2, 3, 4, 5 or 6
p is: 1 or 2;
r is: 1, 2, 3, or 4;
R¹ and R² are each independently:
   hydrogen,
   haloalkyl,
   C₁-C₆ alkyl,
   (CH₂)ₙC₃-C₈ cycloalkyl, or
   R¹ and R² form a 4- to 8-membered nonaromatic carbocyclic ring; and wherein at least one of R¹ and R² is alkyl or cycloalkyl, and;
R³ is: hydrogen,
   nitro,
   cyano,
   hydroxyl,
   halo,
   haloalkyl,
   haloalkyloxy,
   aryloxy,
   C₁-C₆ alkyl,
   C₁-C₆ alkoxy, or
   C₃-C₈ cycloalkyl;
R⁴ and R⁵ are each independently: hydrogen or C₁-C₆ alkyl;
R⁶ is: hydrogen, C₁-C₆ alkyl or aminoalkyl;
R^{6A} is: carboxamide, sulfonamide, acylsulfonamide, tetrazole, 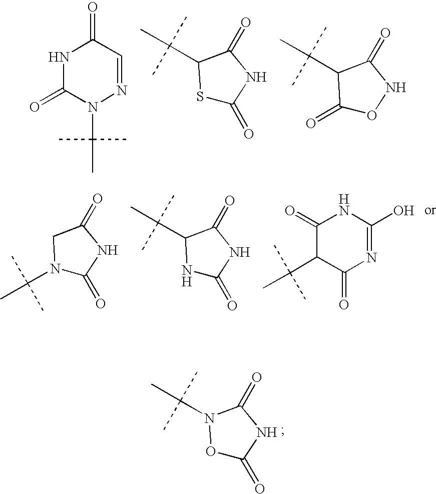
R⁷ is: hydrogen,
   oxo,
   nitro,
   cyano,
   hydroxyl,
   halo,
   haloalkyl,
   haloalkyloxy,
   aryloxy,
   arylalkyl,
   aminoalkyl,
   C₁-C₆ alkyl,
   C₁-C₆ alkoxy,
   (CH₂)ₙC₃-C₈ cycloalkyl,
   C(O)R⁹,
   C(O)OR⁹,
   C(═NOR⁸)R⁹,
   CR⁸(OH)R⁹,
   C[═C(R⁸)₂]R⁹,
   OR⁹,
   SR⁹ or
   S(O)ₚR⁹;
R⁸ is: hydrogen or C₁-C₆ alkyl; and
R⁹ is: hydrogen,
   C₁-C₆ alkyl,
   C₃-C₈ cycloalkyl,
   aryl,
   heteroaryl or
   heterocyclyl,
   wherein alkyl, cycloalkyl, aryl, heteroaryl or heterocyclyl being optionally substituted with one or more substituents selected from the group consisting of:
   hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

A preferred embodiment of the present invention is a compound having a structural formula II, 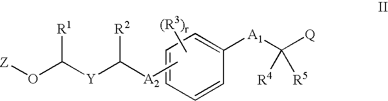
or a pharmaceutically acceptable salt, solvate, hydrate or stereoisomer thereof, wherein:
A₁ is: a bond, CH₂, O or S, and wherein A₁ and R⁴ or A₁ and R⁵ together being a 3- to 6-membered carbocyclyl when A₁ is a carbon;
A₂ is: O or S or CH₂;
Q is: —C(O)OR⁶, or R^{6A};
Y is: a bond, C₁-C₆ alkyl or C₃-C₆ cycloalkyl;
Z is: a) aryl;
   b) a 5- to 10-membered heteroaryl wherein the heteroaryl containing at least one heteroatom selected from N, O or S,
   c) bi-aryl, wherein biaryl being defined as aryl substituted with another aryl or aryl substituted with heteroaryl, or
   d) bi-heteroaryl, wherein bi-heteroaryl being defined as heteroaryl substituted with another heteroaryl, or heteroaryl substituted with aryl, and wherein aryl, heteroaryl, bi-aryl and bi-heteroaryl being optionally substituted with one or more groups independently selected from R⁷;
n is: 1, 2, 3, 4, 5 or 6
p is: 1 or 2;
r is: 1, 2, 3, or 4;
R¹ and R² are each independently:
   hydrogen,
   haloalkyl,
   C₁-C₆ alkyl,
   (CH₂)ₙC₃-C₈ cycloalkyl, or
   R¹ and R² form a 4- to 8-membered nonaromatic carbocyclic ring; and
   wherein at least one of R¹ and R² is alkyl or cycloalkyl, and;
R³ is: hydrogen,
   nitro,
   cyano,
   hydroxyl,
   halo,
   haloalkyl,
   haloalkyloxy,
   aryloxy,
   C₁-C₆ alkyl,
   C₁-C₆ alkoxy, or
   C₃-C₈ cycloalkyl;
R⁴ and R⁵ are each independently: hydrogen or C₁-C₆ alkyl;
R⁶ is: hydrogen, C₁-C₆ alkyl or aminoalkyl;
R^{6A} is: carboxamide, sulfonamide, acylsulfonamide, tetrazole, 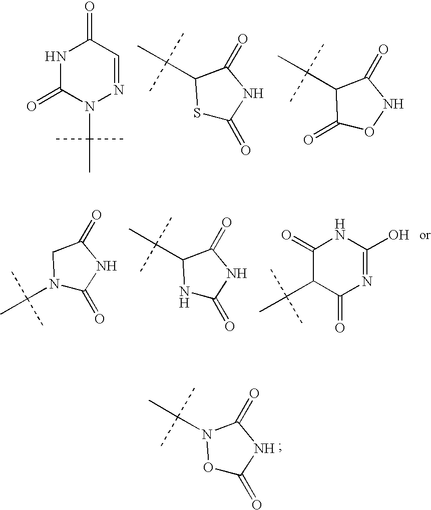
R⁷ is: hydrogen,
   oxo,
   nitro,
   cyano,
   hydroxyl,
   halo,
   haloalkyl,
   haloalkyloxy,
   aryloxy,
   arylalkyl,
   aminoalkyl,
   C₁-C₆ alkyl,
   C₁-C₆ alkoxy,
   (CH₂)ₙC₃-C₈ cycloalkyl,
   C(O)R⁹,
   C(O)OR⁹,
   C(═NOR⁸)R⁹,
   CR⁸(OH)R⁹,
   C[═C(8)₂]R⁹,
   OR⁹,
   SR⁹ or
   S(O)ₚR⁹;
R⁸ is: hydrogen or C₁-C₆ alkyl; and
R⁹ is: hydrogen,
   C₁-C₆ alkyl,
   C₃-C₈ cycloalkyl,
   aryl,
   heteroaryl or
   heterocyclyl,
   wherein alkyl, cycloalkyl, aryl, heteroaryl or heterocyclyl being optionally substituted with one or more substituents selected from the group consisting of:
   hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

The compound as recited above in formula II, wherein Z is optionally substituted phenyl or naphthyl, furanyl, imidazolyl, indolyl, oxazolyl, isoxazolyl, pyridyl, pyrrolyl, thiazolyl, thiophenyl, benzofuranyl, benzothiophenyl, benzoisoxazolyl, quinolinyl, isoquinolinyl or a structural formula selected from following: 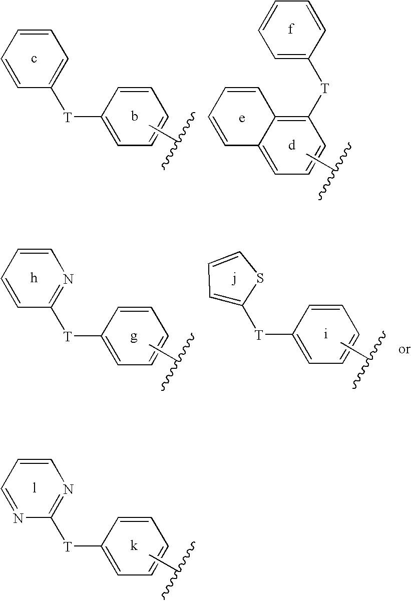
wherein T is:
a bond, —(CH₂)_{q}O—, —O(CH₂)_{q}—, —C(O)(CH₂)_{q}—, —(CH₂)_{q}C(O)—, —(CH₂)_{q}S—, —S(CH₂)_{q}—, S[O]ₚ, —(C₁-C₃ alkyl)-, —(CH₂)_{q}C(═CH₂)—, —C(═CH₂)(CH₂)_{q}—, —(CH₂)_{q}C(═NOH)—, —C(═NOH)(CH₂)_{q}—, —(CH₂)_{q}C(═NOCH₃)—, —C(═NOCH₃)(CH₂)_{q}—, —CH(OH)(CH₂)_{q}—, or —(CH₂)_{q}CH(OH)—,
   q is: 0, 1, 2 or 3; and
   rings b to l are each optionally substituted with one or more groups independently selected from the group consisting of:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Another preferred embodiment of the present invention is a compound having a structural formula III, 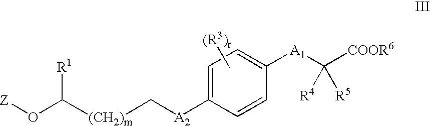
or a pharmaceutically acceptable salt, solvate, hydrate or stereoisomer thereof, wherein m is 1, 2, 3 or 4.

Yet another preferred embodiment of the present invention is the compound having a structural formula IV, 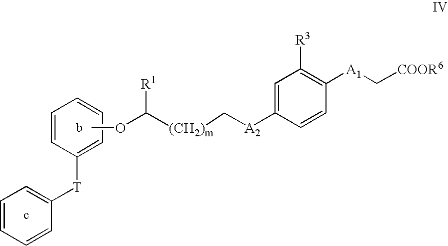
or a pharmaceutically acceptable salt, solvate, hydrate or stereoisomer thereof, wherein:
A₁ and A₂ are respectively:
O and O,
CH₂ and O,
CH₂ and S,
O and S or
S and O;
   m is: 1 or 2;
   R¹ is: C₁-C₃ alkyl;
   R³ is: hydrogen, halo or C₁-C₆ alkyl;
   R⁶ and R⁹ are each independently: hydrogen or C₁-C₆ alkyl;
   T is: a bond, —O—, —C(O)—, —S(O)—S(O)₂—, —C(═CH₂)—, —C(—NOH)— or —CH(OH)—; and
   rings b and c are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Yet another preferred embodiment of the present invention is the compound having a structural formula V, 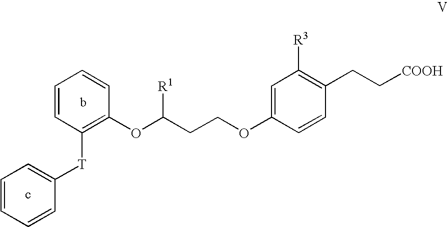
or a pharmaceutically acceptable salt, solvate, hydrate or stereoisomer thereof, wherein:
T is: a bond, —O— or —C(O)—;
R¹ is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, methyl, ethyl, isopropyl, N(CH₃)₂, S(O)₂CH₃, methoxy and cyclopropyl.

Yet another preferred embodiment of the present invention is a compound having a structural formula VI, 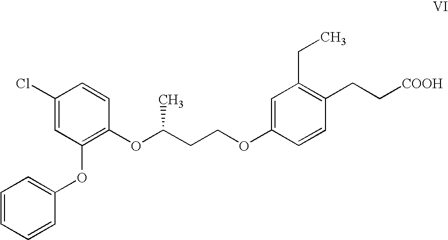
or a pharmaceutically acceptable salt, solvate or hydrate thereof.

Yet another preferred embodiment of the present invention is the compound having a structural formula VII, 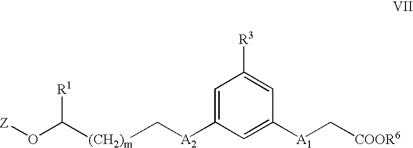
or a pharmaceutically acceptable salt, solvate, hydrate or stereoisomer thereof, wherein:
Z is: 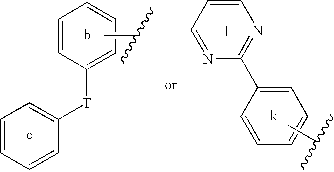
A₁ and A₂ are respectively: bond and S; bond and 0; CH₂ and S; or CH₂ and O;
m is: 1 or 2;
R¹ is: C₁-C₃alkyl;
R³ is: hydrogen, halo or C₁-C₆ alkyl;
R⁶ and R⁹ are each independently: hydrogen or C₁-C₆ alkyl;
T is: bond, —O—, —C(O)—, —S(O)—S(O)₂—, —C(═CH₂)—, —C(═NOH)— or —CH(OH)—; and
rings b, c, k and l are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

The compound as recited above in formula VII, wherein R¹ is: methyl, ethyl or cyclopropyl; R³ is: methyl or ethyl; and rings b, c k and l are each optionally substituted with one or more substituent independently selected from the group consisting of: hydrogen, Cl, Br, CF₃, OCF₃, N(CH₃)₂, S(O)₂CH₃, methyl, ethyl, isopropyl, methoxy and cyclopropyl.

Yet another preferred embodiment of the present invention is the compound having a structural formula VIII, 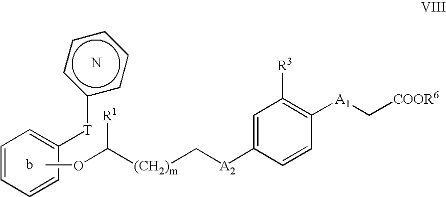
or a pharmaceutically acceptable salt, solvate, hydrate or stereoisomer thereof, wherein:
A₁ and A₂ are respectively:
O and O,
CH₂ and O,
CH₂ and S,
O and S or
S and O;
   m is: 1 or 2;
   R¹ is: C₁-C₃ alkyl; and
   R³ is: hydrogen, halo or C₁-C₆ alkyl;
   R⁶ and R⁹ are each independently: hydrogen or C₁-C₆ alkyl;
   T is: a bond, —O—, —C(O)—, —S(O)—S(O)₂—, —C(═CH₂)—, —C(═NOH)— or —CH(OH)—; and
   ring b is optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Yet another preferred embodiment of the present invention is a compound having a structural formula IX, 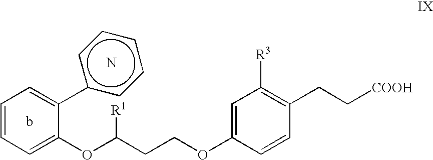
or a pharmaceutically acceptable salt, solvate, hydrate or stereoisomer thereof, wherein:
R¹ is: C₁-C₃ alkyl;
R³ is: hydrogen, halo or C₁-C₄ alkyl;
ring b is optionally substituted with one or more groups independently selected from the group consisting of: hydrogen, halo, haloalkyl, haloalkyloxy and C₁-C₆ alkyl.

Yet another preferred embodiment of the present invention is the compound having a structural formula X, 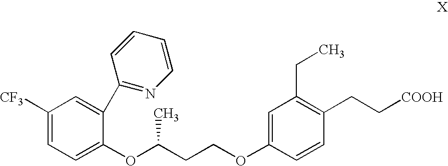
or a pharmaceutically acceptable salt, solvate or hydrate thereof.

Yet another preferred embodiment of the present invention is the compound having a structural formula XI, 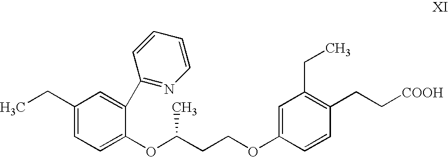
or a pharmaceutically acceptable salt, solvate or hydrate thereof.

Yet another preferred embodiment of the present invention is the compound having a structural formula XII, 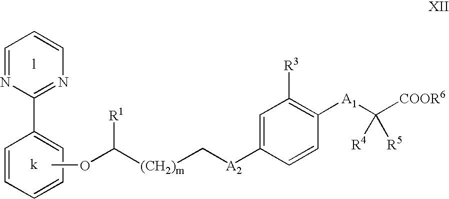
or a pharmaceutically acceptable salt, solvate, hydrate or stereoisomer thereof, wherein:
A₁ and A₂ are respectively:
O and O,
CH₂ and O,
CH₂ and S,
O and S or
S and O;
   m is: 1 or 2;
   R¹ is: C₁-C₃ alkyl; and
   R³ is: hydrogen, halo or C₁-C₆ alkyl;
   R⁴, R⁵, R⁶ and R⁹ are each independently: hydrogen or C₁-C₆ alkyl;
   rings k and l are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

The compound as recited above in formula XII, wherein R⁴ and R⁵ are each methyl or ethyl; m is 1; rings k and l are each optionally substituted with one or more substituent independently selected from the group consisting of: hydrogen, Cl, Br, CF₃, OCF₃, N(CH₃)₂, S(O)₂CH₃, methyl, ethyl, isopropyl, methoxy and cyclopropyl; and oxygen atom oxygen atom of —O—CH(R¹)—(CH₂)ₘ— moiety is placed in an ortho position relative to the ring l.

Yet another preferred embodiment of the present invention is the compound having a structural formula XIII, 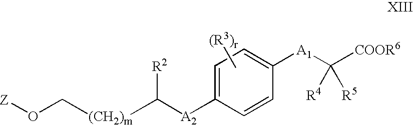
or a pharmaceutically acceptable salt, solvate, hydrate or stereoisomer thereof, wherein m is 1, 2, 3, or 4.

Yet another preferred embodiment of the present invention is the compound having a structural formula XIV, 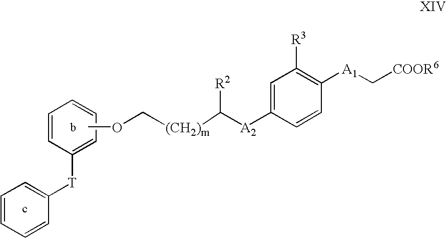
or a pharmaceutically acceptable salt, solvate, hydrate or stereoisomer thereof, wherein:
A₁ and A₂ are respectively:
O and O,
CH₂ and O,
CH₂ and S,
O and S, or
S and O;
   m is: 1 or 2:
   R² is: C₁-C₃ alkyl; and
   R³ is: hydrogen, halo or C₁-C₆ alkyl;
   R⁶ and R⁹ are each independently: hydrogen or C₁-C₆ alkyl;
   T is: a bond, —O—, —C(O)—, —S(O)—S(O)₂—, —C(═CH₂)—, —C(═NOH)— or —CH(OH)—; and
   rings b and c are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Yet another preferred embodiment of the present invention is the compound having a structural formula XV, 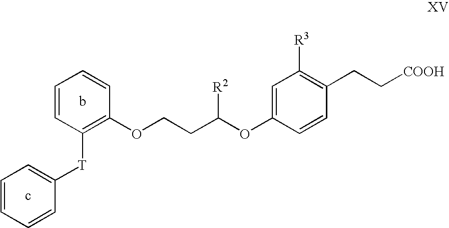
or a pharmaceutically acceptable salt, solvate, hydrate or stereoisomer thereof, wherein:
T is: a bond, O or C(O);
R² is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, N(CH₃)₂, S(O)₂CH₃, methyl, ethyl, isopropyl, methoxy and cyclopropyl.

Yet another preferred embodiment of the present invention is the compound having a structural formula XVI, 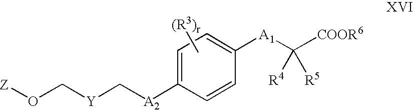
or a pharmaceutically acceptable salt, solvate, hydrate or stereoisomer thereof, wherein Y is a branched alkyl or C₃-C₆ cycloalkyl.

Yet another preferred embodiment of the present invention is the compound having a structural formula XVII, 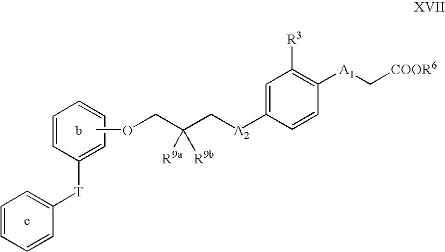
or a pharmaceutically acceptable salt, solvate, hydrate or stereoisomer thereof, wherein:
A₁ and A₂ are respectively:
O and O,
CH₂ and O,
CH₂ and S,
O and S, or
S and O;
   R³ is: hydrogen, halo or C₁-C₆ alkyl;
   R⁶ and R⁹ are each independently: hydrogen or C₁-C₄ alkyl;
   R^{9a} and R^{9b} are:
each independently hydrogen or C₁-C₄ alkyl wherein at least one of R^{9a} and R^{9b} being C₁-C₄ alkyl, or together C₃-C₆ cycloalkyl;
   T is: a bond, —O—, —C(O)—, —S(O)—S(O)₂—, —C(═CH₂)—, —C(═NOH)— or —CH(OH)—; and
   rings b and c are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Yet another embodiment of the present invention is the compound having a structural formula XVIII, 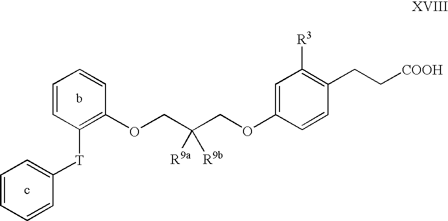
or a pharmaceutically acceptable salt, solvate, hydrate or stereoisomer thereof, wherein:
T is: a bond, O or C(O);
R³ is: methyl or ethyl;
R^{9a} and R^{9b} are each independently hydrogen, methyl or ethyl, wherein at least one of R^{9a} and R^{9b} being methyl or ethyl;
rings b and c are each optionally substituted with one or more substituent independently selected from the group consisting of: hydrogen, Cl, Br, CF₃, OCF₃, S(O)₂CH₃, N(CH₃)₂, methyl, ethyl, isopropyl, methoxy and cyclopropyl.

Yet another preferred embodiment of the present invention is the compound having a structural formula XIX, 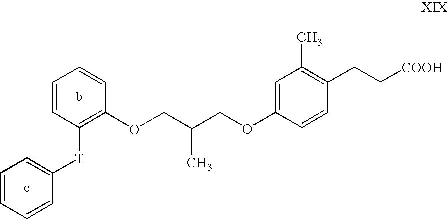
or a pharmaceutically acceptable salt, solvate, hydrate or stereoisomer thereof.

Yet another preferred embodiment of the present invention is the compound having a structural formula XX, 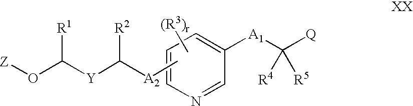
or a pharmaceutically acceptable salt, solvate, hydrate or stereoisomer thereof, wherein:
A₁ is: a bond, CH₂, O or S, and
wherein A₁ and R⁴ or A₁ and R⁵ together being a 3- to 6-membered carbocyclyl when A₁ is a carbon;
   A₂ is: O or S or CH₂;
   Q is: —C(O)OR⁶, or R^{6A};
   Y is: a bond, C₁-C₆ alkyl or C₃-C₆ cycloalkyl;
   Z is: a) aryl;
b) a 5- to 10-membered heteroaryl wherein the heteroaryl containing at least one heteroatom selected from N, O or S,
c) bi-aryl, wherein biaryl being defined as aryl substituted with another aryl or aryl substituted with heteroaryl, or
d) bi-heteroaryl, wherein bi-heteroaryl being defined as heteroaryl substituted with another heteroaryl, or heteroaryl substituted with aryl, and wherein aryl, heteroaryl, bi-aryl and bi-heteroaryl being optionally substituted with one or more groups independently selected from R⁷;
   n is: 1, 2, 3, 4, 5 or 6
   p is: 1 or 2;
   r is: 1, 2, 3, or 4;
   R¹ and R² are each independently:
hydrogen,
haloalkyl,
C₁-C₆ alkyl,
(CH₂)ₙC₃-C₈ cycloalkyl, or
R¹ and R² form a 4- to 8-membered nonaromatic carbocyclic ring; and
wherein at least one of R¹ and R² is alkyl or cycloalkyl, and;
   R³ is: hydrogen,
nitro,
cyano,
hydroxyl,
halo,
haloalkyl,
haloalkyloxy,
aryloxy,
C₁-C₆ alkyl,
C₁-C₆ alkoxy, or
C₃-C₈ cycloalkyl;
   R⁴ and R⁵ are each independently: hydrogen or C₁-C₆ alkyl;
   R⁶ is: hydrogen, C₁-C₆ alkyl or aminoalkyl;
   R^{6A} is: carboxamide, sulfonamide, acylsulfonamide, tetrazole, 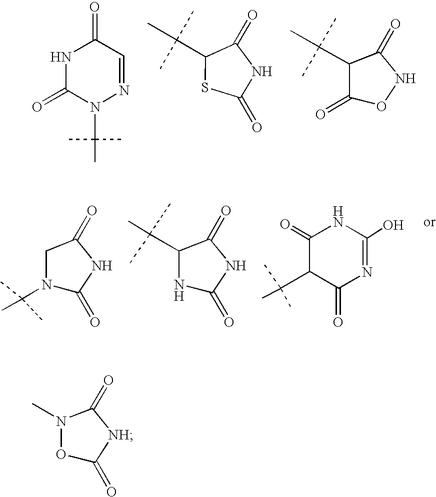
   R⁷ is: hydrogen,
oxo,
nitro,
cyano,
hydroxyl,
halo,
haloalkyl,
haloalkyloxy,
aryloxy,
arylalkyl,
aminoalkyl,
C₁-C₆ alkyl,
C₁-C₆ alkoxy,
(CH₂)ₙC₃-C₈ cycloalkyl,
C(O)R⁹,
C(O)OR⁹,
C(═NOR⁸)R⁹,
CR⁸(OH)R⁹,
C[═C(R⁸)₂]R⁹,
OR⁹,
SR⁹ or
S(O)ₚR⁹;
   R⁸ is: hydrogen or C₁-C₆ alkyl; and
   R⁹ is: hydrogen,
C₁-C₆ alkyl,
C₃-C₈ cycloalkyl,
aryl,
heteroaryl or
heterocyclyl,
wherein alkyl, cycloalkyl, aryl, heteroaryl or heterocyclyl being optionally substituted with one or more substituents selected from the group consisting of:
hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

The compound as recited above in formula XX, wherein Z is optionally substituted phenyl or naphthyl, furanyl, imidazolyl, indolyl, oxazolyl, isoxazolyl, pyridyl, pyrrolyl, thiazolyl, thiophenyl, benzofuranyl, benzothiophenyl, benzoisoxazolyl, quinolinyl, isoquinolinyl or a structural formula selected from following: 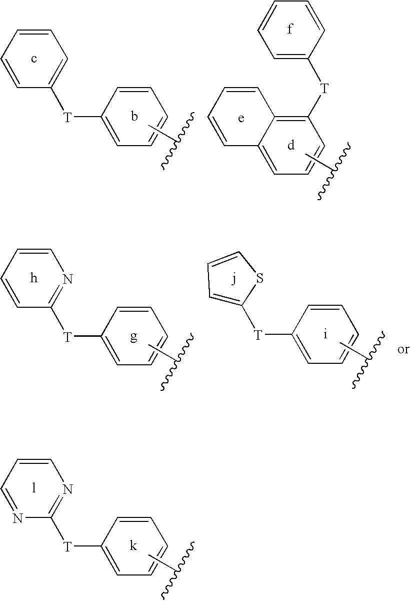
wherein T is:
a bond, —(CH₂)_{q}O—, —O(CH₂)_{q}—, —C(O)(CH₂)_{q}—, —(CH₂)_{q}C(O)—, —(CH₂)_{q}S—, —S(CH₂)_{q}—, S[O]ₚ, —(C₁-C₃ alkyl)-, —(CH₂)_{q}C(═CH₂)—, —C(═CH₂)(CH₂)_{q}—, —(CH₂)_{q}C(═NOH)—, —C(═NOH)(CH₂)_{q}—, —(CH₂)_{q}C(═NOCH₃)—, —C(═NOCH₃)(CH₂)_{q}—, —CH(OH)(CH₂)_{q}—, or —(CH₂)_{q}CH(OH)—,
   q is: 0, 1, 2 or 3; and
   rings b to j are each optionally substituted with one or more groups independently selected from the group consisting of:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Yet another preferred embodiment of the present invention is the compound having a structural formula XXI, 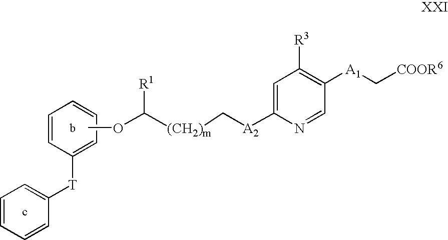
or a pharmaceutically acceptable salt, solvate, hydrate or stereoisomer thereof, wherein:
A₁ and A₂ are respectively:
O and O,
CH₂ and O,
CH₂ and S,
O and S or
S and O;
   m is: 1, 2, 3 or 4;
   R¹ is: C₁-C₃ alkyl; and
   R³ is: hydrogen, halo or C₁-C₆ alkyl;
   R⁶ and R⁹ are each independently: hydrogen or C₁-C₆ alkyl;
   T is: a bond, —O—, —C(O)—, —S(O)—S(O)₂—, —C(═CH₂)—, —C(═NOH)— or —CH(OH)—; and
   rings b and c are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

Yet another preferred embodiment of the present invention is the compound having a structural formula XXII, 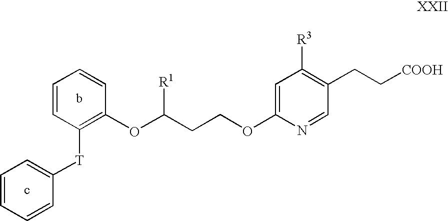
or a pharmaceutically acceptable salt, solvate, hydrate or stereoisomer thereof, wherein:
T is: a bond, —O— or —C(O)—;
R¹ is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, S(O)₂CH₃, N(CH₃)₂, methyl, ethyl, isopropyl, methoxy and cyclopropyl.

Yet another preferred embodiment of the present invention is the compound having a structural formula XXIII, 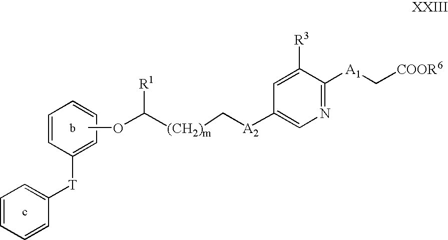
or a pharmaceutically acceptable salt, solvate, hydrate or stereoisomer thereof, wherein:
A₁ and A₂ are respectively:
O and O,
CH₂ and O,
CH₂ and S,
O and S or
S and O;
   m is: 1, 2, 3 or 4;
   R¹ is: C₁-C₃ alkyl; and
   R³ is: hydrogen, halo or C₁-C₆ alkyl;
   R⁶ and R⁹ are each independently: hydrogen or C₁-C₆ alkyl;
   T is: a bond, —O—, —C(O)—, —S(O)—S(O)₂—, —C(═CH₂)—, —C(═NOH)— or —CH(OH)—; and
   rings b and c are each optionally substituted with one or more groups independently selected from:
hydrogen, oxo, nitro, cyano, hydroxyl, halo, haloalkyl, haloalkyloxy, aryloxy, arylalkyl, amin [symbol] R⁹, C₁-C₆ alkyl, C₁-C₆ alkoxy and (CH₂)ₙC₃-C₈ cycloalkyl.

Yet another preferred embodim [symbol] present invention is the compound having a structural formula XXIV, 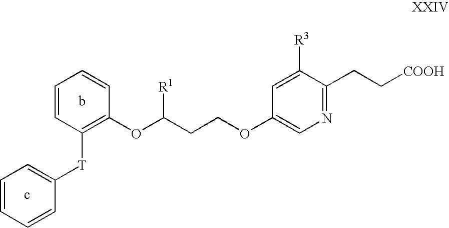
or a pharmaceutically acceptable salt, solvate, hydrate or stereoisomer thereof, wherein:
T is: a bond, —O— or —C(O)—;
R¹ is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, S(O)₂CH₃, N(CH₃)₂, methyl, ethyl, isopropyl, methoxy and cyclopropyl.

The more preferred embodiment of the present invention is the compounds listed below, more specifically the compounds of PPAR gamma/delta dual agonists:

| No. | Structure | Name |
|---|---|---|
| 1 | 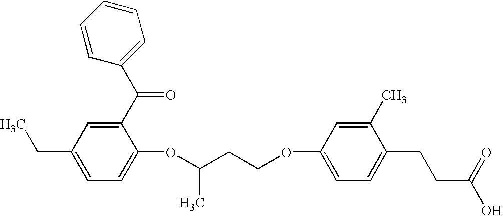 | 3-{4-[3-(2-Benzoyl-4-ethyl-phenoxy)- butoxy]-2-methyl-phenyl}-propionic acid |
| 2 | 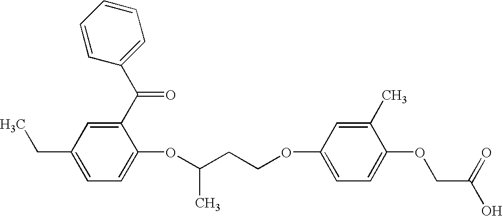 | {4-[3-(2-Benzoyl-4-ethyl-phenoxy)- butoxy]-2-methyl-phenoxy}-acetic acid |
| 3 | 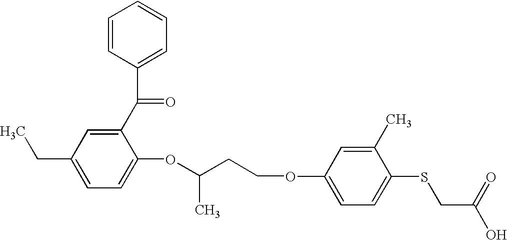 | {4-[3-(2-Benzoyl-4-ethyl-phenoxy)- butoxy]-2-methyl-phenylsulfanyl}-acetic acid |
| 4 | 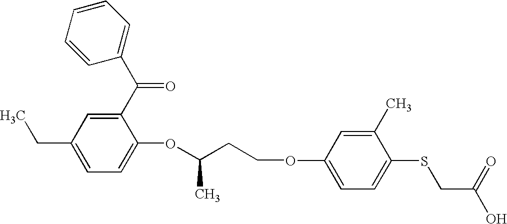 | {4-[3-(2-Benzoyl-4-ethyl-phenoxy)- butoxy]-2-methyl-phenylsulfanyl}-acetic acid |
| 5 | 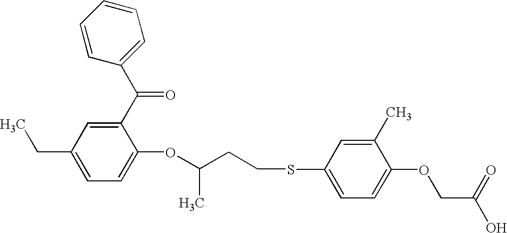 | {4-[3-(2-Benzoyl-4-ethyl-phenoxy)- butylsulfanyl]-2-methyl-phenoxy}-acetic acid |
| 6 | 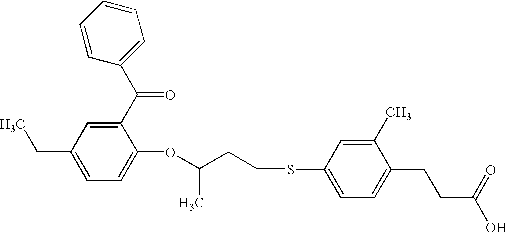 | {4-[3-(2-Benzoyl-4-ethyl-phenoxy)- butylsulfanyl]-2-methyl-phenyl}-propionic acid |
| 7 | 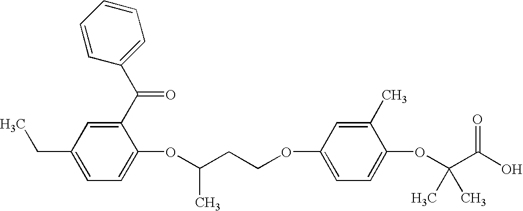 | 2-{4-[3-(2-Benzoyl-4-ethyl-phenoxy)- butoxy]-2-methyl-phenoxy}-2-methyl- propionic acid |
| 8 | 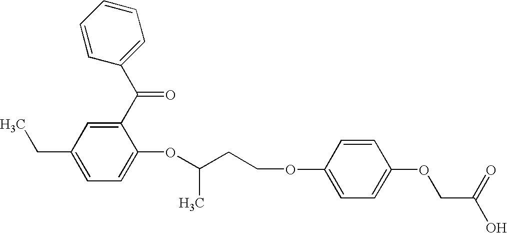 | {4-[3-(2-Benzoyl-4-ethyl-phenoxy)- butoxy]-phenoxy}-acetic acid |
| 9 | 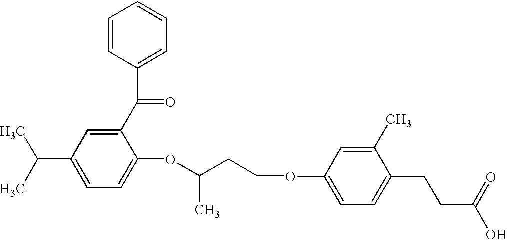 | 3-{4-[3-(2-Benzoyl-4-isopropyl-phenoxy)- butoxy]-2-methyl-phenyl)}-propionic acid |
| 10 | 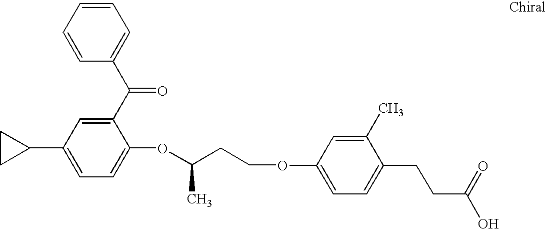 | 3-{4-[3-(2-Benzoyl-4-cyclopropyl-phenoxy)- butoxy]-2-methyl-phenyl}-propionic acid |
| 11 | 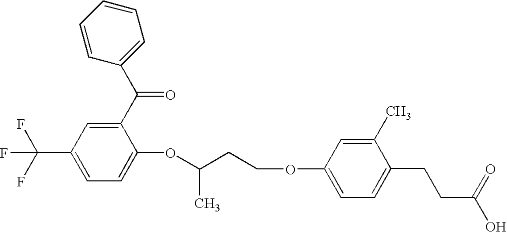 | 3-{4-[3-(2-Benzoyl-4-trifluoromethyl- phenoxy)-butoxy]-2-methyl-phenyl}- propionic acid |
| 12 | 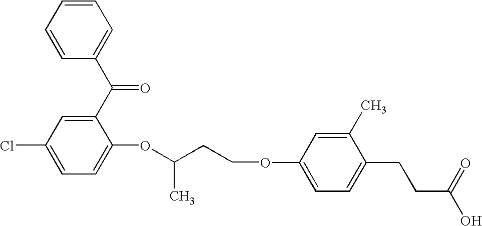 | 3-{4-[3-(2-Benzoyl-4-chloro-phenoxy)- butoxy]-2-methyl-phenyl}-propionic acid |
| 13 | 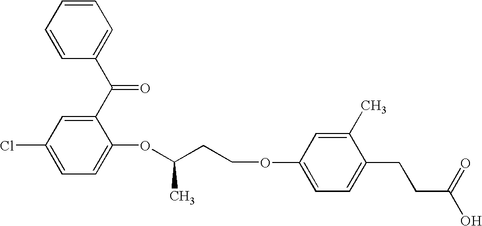 | 3-{4-[3-(2-Benzoyl-4-chloro-phenoxy)- butoxy]-2-methyl-phenyl}-propionic acid |
| 14 | 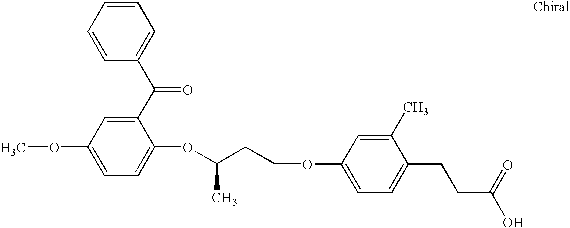 | 3-{4-[3-(2-Benzoyl-4-methoxy-phenoxy)- butoxy]-2-methyl-phenyl}-propionic acid |
| 15 | 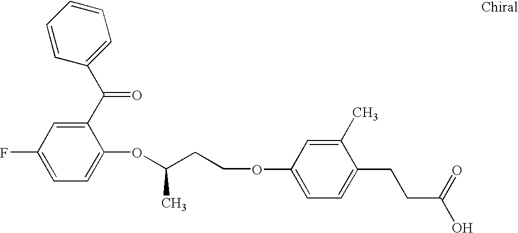 | 3-{4-[3-(2-Benzoyl-4-fluoro-phenoxy)- butoxy]-2-methyl-phenyl}-propionic acid |
| 16 | 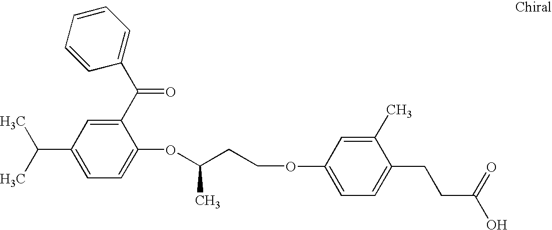 | 3-{4-[3-(2-Benzoyl-4-isopropyl-phenoxy)- butoxy]-2-methyl-phenyl}-propionic acid |
| 17 | 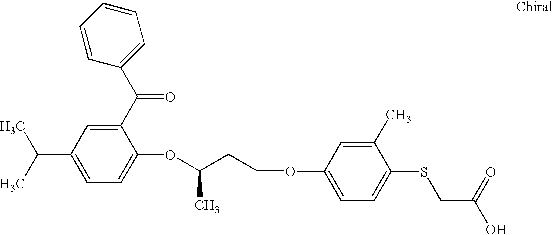 | {4-[3-(2-Benzoyl-4-isopropyl-phenoxy)- butoxy]-2-methyl-phenylsulfanyl}-acetic acid |
| 18 | 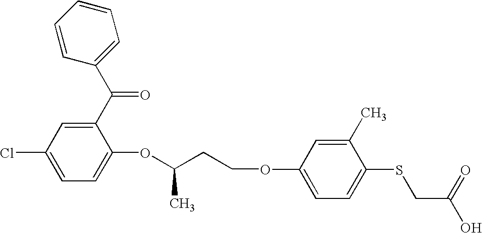 | {4-[3-(2-Benzoyl-4-chloro-phenoxy)- butoxy]-2-methyl-phenylsulfanyl}-acetic acid |
| 19 | 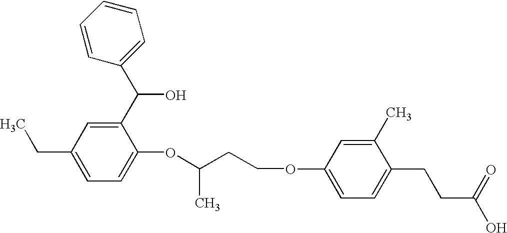 | 3-(4-{3-[4-Ethyl-2-(hydroxy-phenyl-methyl)- phenoxy]-butoxy}-2-methyl-phenyl)- propionic acid |
| 20 | 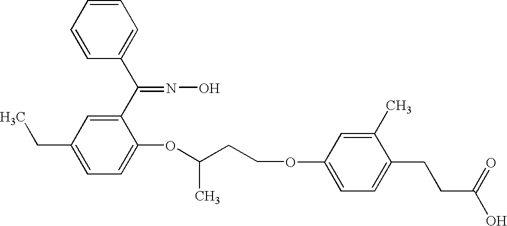 | 3-(4-{3-[4-Ethyl-2-(hydroxyimino-phenyl- methyl)-phenoxy]-butoxy}-2-methyl- phenyl)-propionic acid |
| 21 | 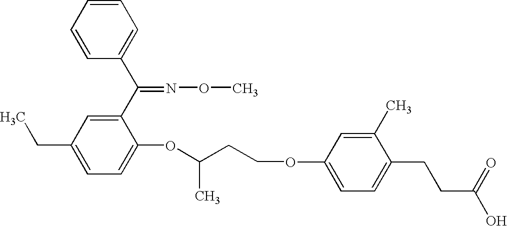 | 3-(4-{3-[4-Ethyl-2-(methoxyimino-phenyl- methyl)-phenoxy]-butoxy}-2-methyl- phenyl)-propionic acid |
| 22 | 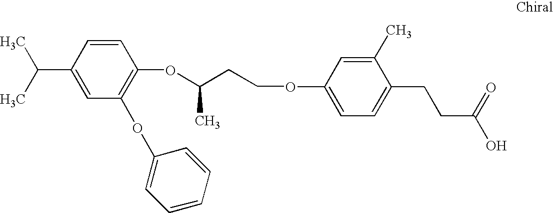 | 3-{4-[3-(4-Isopropyl-2-phenoxy-phenoxy)- butoxy]-2-methyl-phenyl}-propionic acid |
| 23 | 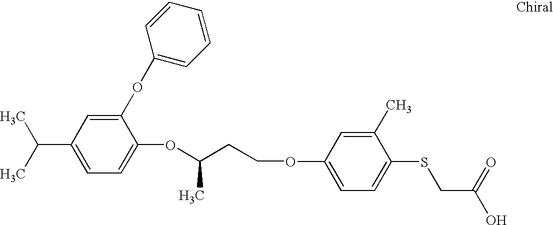 | {4-[3-(4-Isopropyl-2-phenoxy-phenoxy)- butoxy]-2-methyl-phenylsulfanyl}-acetic acid |
| 24 | 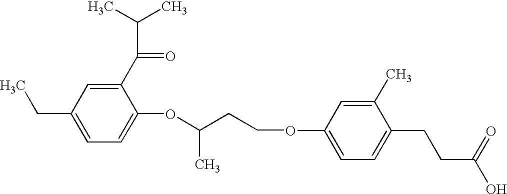 | 3-{4-[3-(4-Ethyl-2-isobutyryl-phenoxy)- butoxy]-2-methyl-phenyl}-propionic acid |
| 25 | 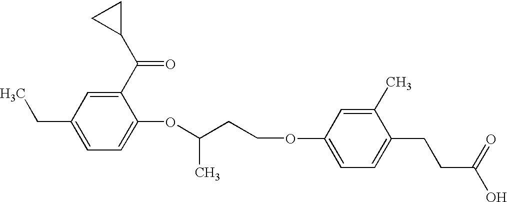 | 3-{4-[3-(2-Cyclopropanecarbonyl-4-ethyl- phenoxy)-butoxy]-2-methyl-phenyl}- propionic acid |
| 26 | 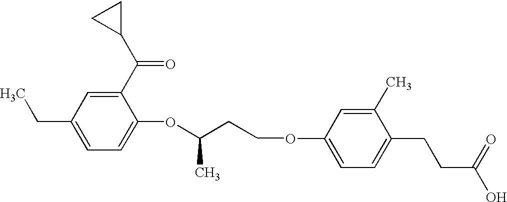 | 3-{4-[3-(2-Cyclopropanecarbonyl-4-ethyl- phenoxy)-butoxy]-2-methyl-phenyl}- propionic acid |
| 27 | 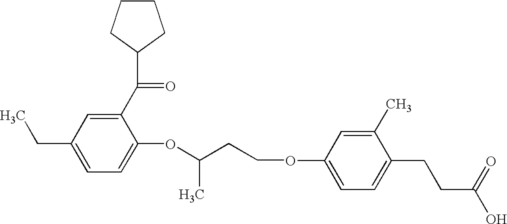 | 3-{4-[3-(2-Cyclopentanecarbonyl-4-ethyl- phenoxy)-butoxy]-2-methyl-phenyl}- propionic acid |
| 28 | 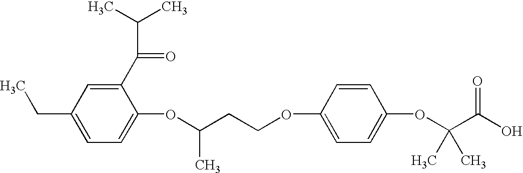 | 2-{4-[3-(4-Ethyl-2-isobutyryl-phenoxy)- butoxy]-phenoxy}-2-methyl-propionic acid |
| 29 | 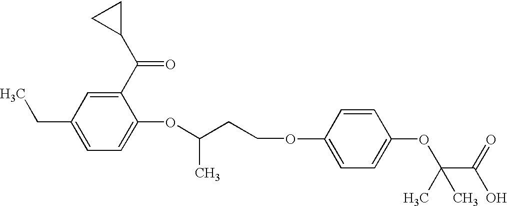 | 2-{4-[3-(2-Cyclopropanecarbonyl-4-ethyl- phenoxy)-butoxy]-phenoxy}-2-methyl- propionic acid |
| 30 | 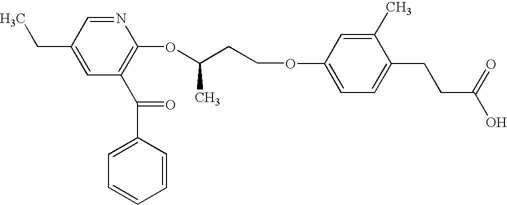 | 3-{4-[3-(3-Benzoyl-5-ethyl-pyridin-2- yloxy)-butoxy]-2-methyl-phenyl}- propionic acid |
| 31 | 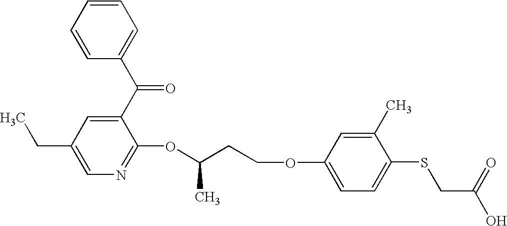 | {4-[3-(3-Benzoyl-5-ethyl-pyridin-2- yloxy)-butoxy]-2-methyl-phenylsulfanyl}- acetic acid |
| 32 | 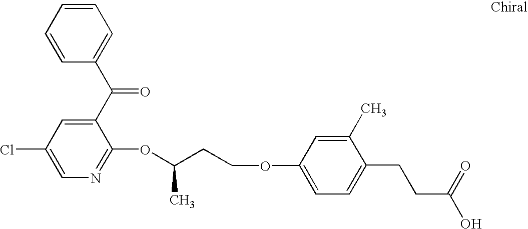 | 3-{4-[3-(3-Benzoyl-5-chloro-pyridin-2- yloxy)-butoxy]-2-methyl-phenyl}- propionic acid |
| 33 | 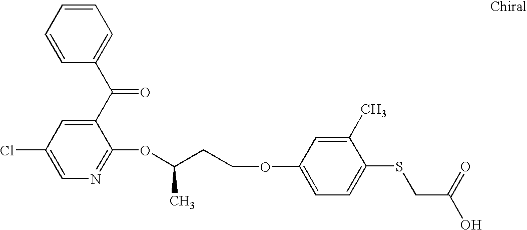 | {4-[3-(3-Benzoyl-5-chloro-pyridin-2- yloxy)-butoxy]-2-methyl-phenylsulfanyl}- acetic acid |
| 34 | 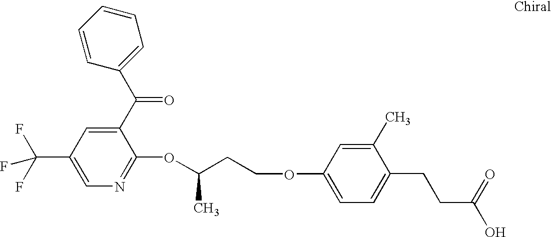 | 3-{4-[3-(3-Benzoyl-5-trifluoromethyl- pyridin-2-yloxy)-butoxy]-2-methyl- phenyl}- propionic acid |
| 35 | 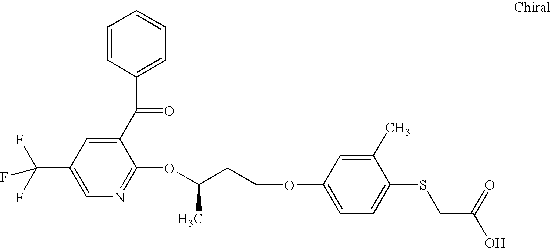 | {4-[3-(3-Benzoyl-5-trifluoromethyl- pyridin-2-yloxy)-butoxy]-2-methyl- phenylsulfanyl}-acetic acid |
| 36 | 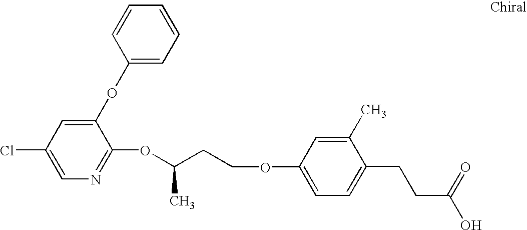 | 3-{4-[3-(5-Chloro-3-phenoxy-pyridin- 2-yloxy)-butoxy]-2-methyl-phenyl}- propionic acid |
| 37 | 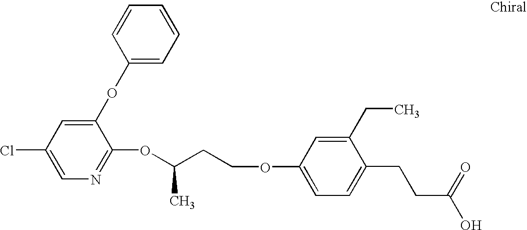 | 3-{4-[3-(5-Chloro-3-phenoxy-pyridin- 2-yloxy)-butoxy]-2-ethyl-phenyl}- propionic acid |
| 38 | 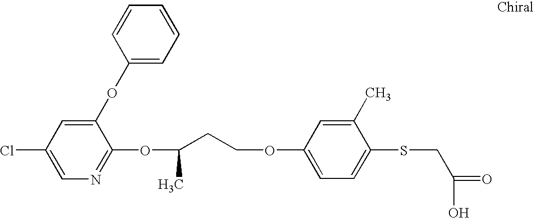 | {4-[3-(5-Chloro-3-phenoxy-pyridin- 2-yloxy)-butoxy]-2-methyl-phenylsulfanyl}- acetic acid |
| 39 | 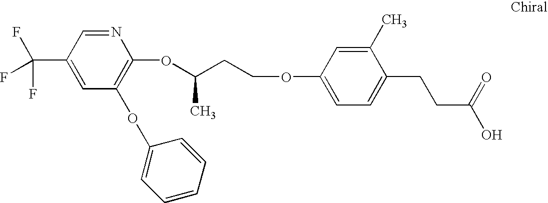 | 3-{2-Methyl-4-[3-(3-phenoxy-5- trifluoromethyl-pyridin-2-yloxy)-butoxy]- phenyl}-propionic acid |
| 40 | 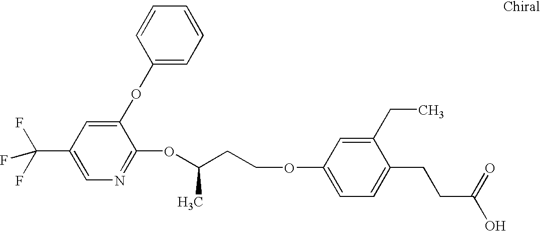 | 3-{2-Ethyl-4-[3-(3-phenoxy-5- trifluoromethyl-pyridin-2-yloxy)-butoxy]- phenyl}-propionic acid |
| 41 | 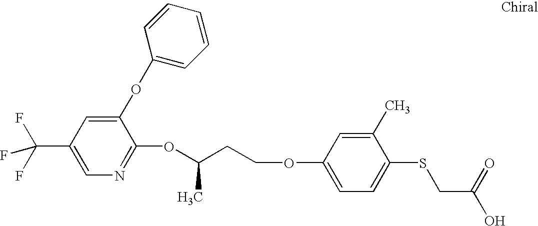 | 3-{2-Ethyl-4-[3-(3-phenoxy-5- trifluoromethyl-pyridin-2-yloxy)-butoxy]- phenyl}-propionic acid |
| 42 | 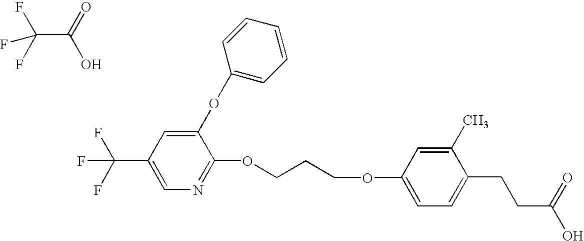 | 3-{2-Methyl-4-[3-(3-phenoxy-5- trifluoromethyl-pyridin-2-yloxy)-propoxy]- phenyl}-propionic acid (trifluoroacetic acid salt) |
| 43 | 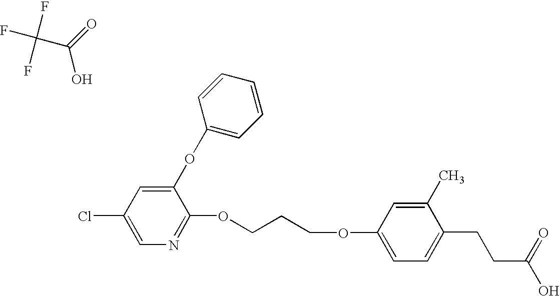 | 3-{4-[3-(5-Chloro-3-phenoxy-pyridin- 2-yloxy)-propoxy]-2-methyl-phenyl}- propionic acid |
| 44 | 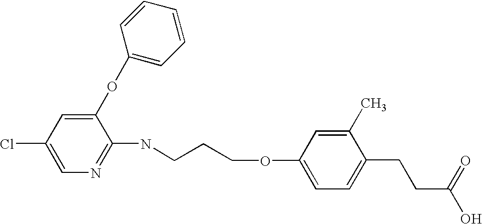 | 3-{4-[2-(5-Chloro-3-phenoxy-pyridin- 2-ylamino)-ethoxy]-2-methyl-phenyl}- propionic acid |
| 45 | 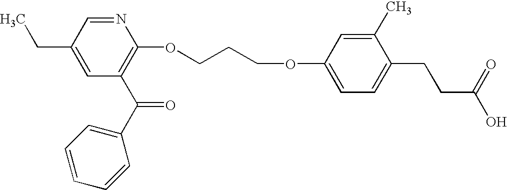 | 3-{4-[3-(3-Benzoyl-5-ethyl-pyridin-2-yloxy)- propoxy]-2-methyl-phenyl}-propionic acid |
| 46 | 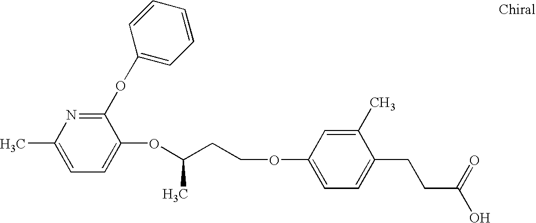 | 3-{2-Methyl-4-[3-(6-methyl-2-phenoxy- pyridin-3-yloxy)-butoxy]-phenyl}- propionic acid |
| 47 | 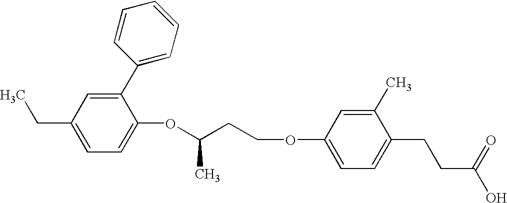 | 3-{4-[3-(5-Ethyl-biphenyl-2-yloxy)-butoxy]- 2-methyl-phenyl}-propionic acid |
| 48 | 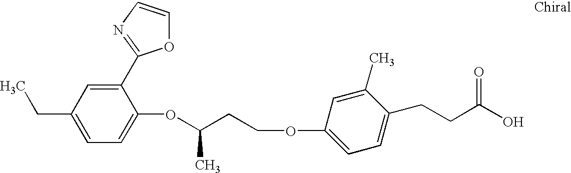 | 3-{4-[3-(4-Ethyl-2-oxazol-2-yl-phenoxy)- butoxy]-2-methyl-phenyl}-propionic acid |
| 49 | 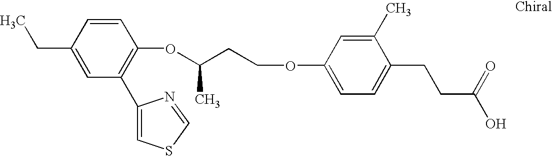 | 3-{4-[3-(4-Ethyl-2-thiazol-4-yl-phenoxy)- phenoxy)-butoxy]-2-methyl-phenyl}- propionic acid |
| 50 | 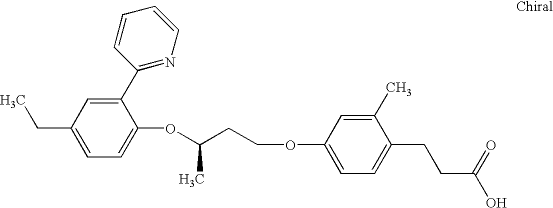 | 3-{4-[3-(4-Ethyl-2-pyridin-2-yl-phenoxy)- butoxy]-2-methyl-phenyl}-propionic acid |
| 51 | 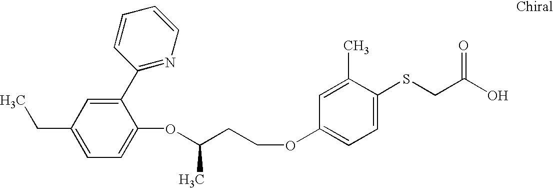 | {4-[3-(4-Ethyl-2-pyridin-2-yl-phenoxy)- butoxy]-2-methyl-phenylsulfanyl}-acetic acid |
| 52 | 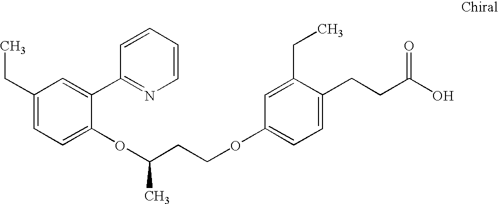 | 3-{2-Ethyl-4-[3-(4-ethyl-2-pyridin-2-yl- phenoxy)-butoxy]-phenyl}-propionic acid |
| 53 | 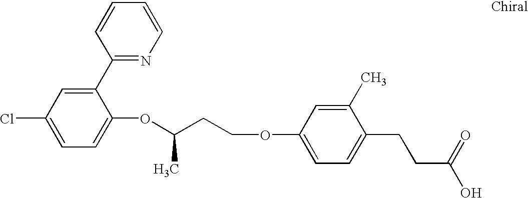 | 3-{4-[3-(4-Chloro-2-pyridin-2-yl-phenoxy)- butoxy]-2-methyl-phenyl}-propionic acid |
| 54 | 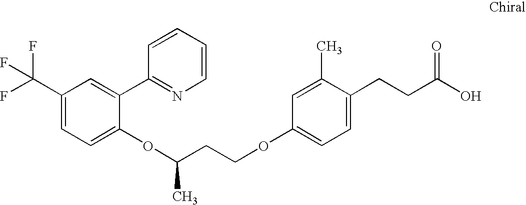 | 3-{2-Methyl-4-[3-(2-pyridin-2-yl-4- trifluoromethyl-phenoxy)-butoxy]-phenyl}- propionic acid |
| 55 | 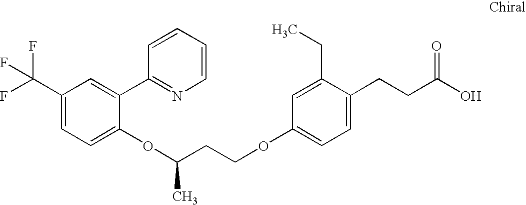 | 3-{2-Ethyl-4-[3-(2-pyridin-2-yl-4- trifluoromethyl-phenoxy)-butoxy]-phenyl}- propionic acid |
| 56 | 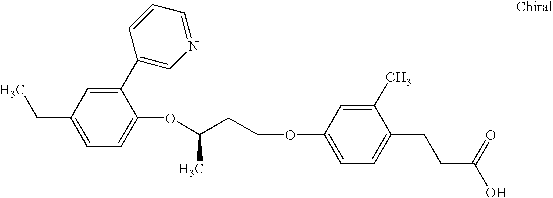 | 3-{4-[3-(4-Ethyl-2-pyridin-3-yl-phenoxy)- butoxy]-2-methyl-phenyl}-propionic acid |
| 57 | 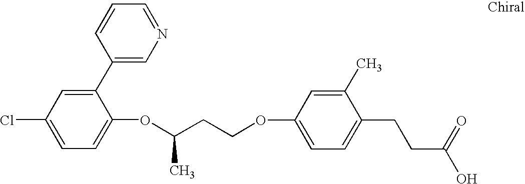 | 3-{4-[3-(4-Chloro-2-pyridin-3-yl-phenoxy)- butoxy]-2-methyl-phenyl}-propionic acid |
| 58 | 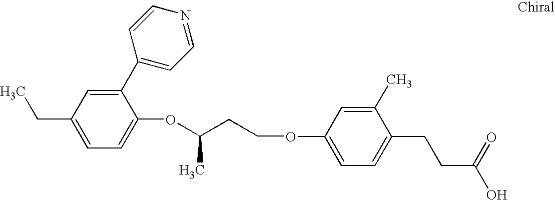 | 3-{4-[3-(4-Ethyl-2-pyridin-4-yl-phenoxy)- butoxy]-2-methyl-phenyl}-propionic acid |
| 59 | 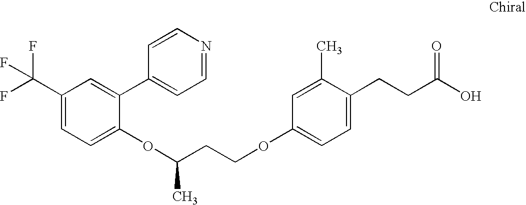 | 3-{2-Methyl-4-[3-(2-pyridin-4-yl-4- trifluoromethyl-phenoxy)-butoxy]-phenyl}- propionic acid |
| 60 | 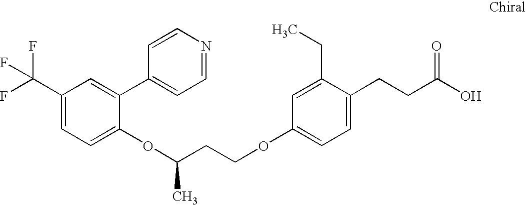 | 3-{2-Ethyl-4-[3-(2-pyridin-4-yl-4- trifluoromethyl-phenoxy)-butoxy]-phenyl}- propionic acid |
| 61 | 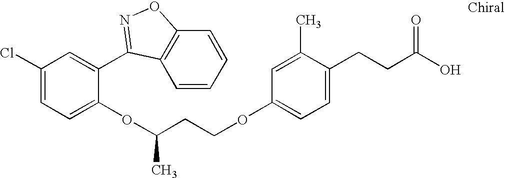 | 3-{4-[3-(2-Benzo[d]isoxazol-3-yl-4-chloro- phenoxy)-butoxy]-2-methyl-phenyl}- propionic acid |
| 62 | 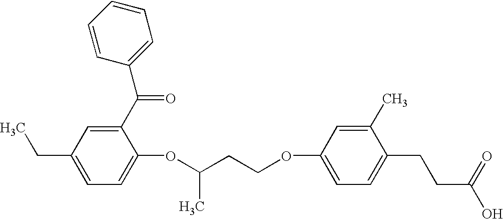 | 3-{4-[3-(2-Benzoyl-4-ethyl-phenoxy)- butoxy]-2-methyl-phenyl}-propionic acid |
| 63 | 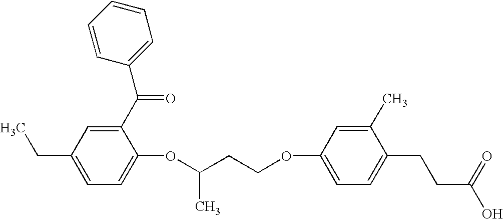 | {4-[3-(2-Benzoyl-4-ethyl-phenoxy)- butoxy]-2-methyl-phenoxy}-acetic acid |
| 64 | 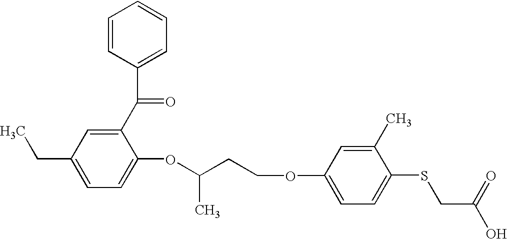 | {4-[3-(2-Benzoyl-4-ethyl-phenoxy)- butoxy]-2-methyl-phenylsulfanyl}-acetic acid |
| 65 | 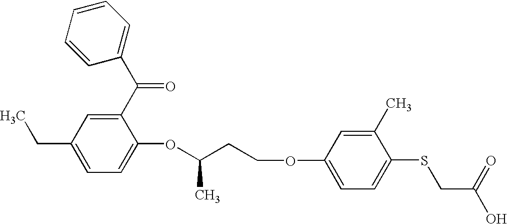 | {4-[3-(2-Benzoyl-4-ethyl-phenoxy)- butoxy]-2-methyl-phenylsulfanyl}-acetic acid |
| 66 | 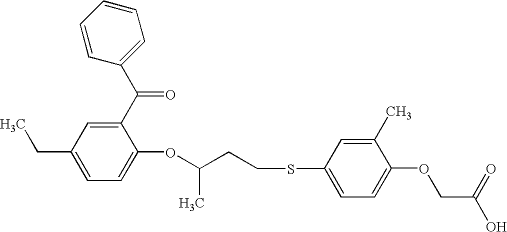 | {4-[3-(2-Benzoyl-4-ethyl-phenoxy)- butylsulfanyl]-2-methyl-phenoxy}-acetic acid |
| 67 | 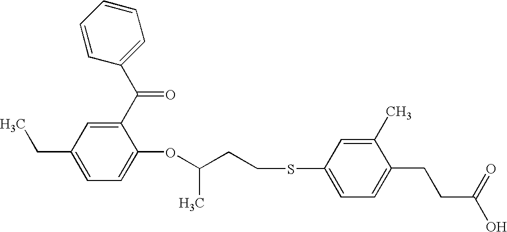 | 3-{4-[3-(2-Benzoyl-4-ethyl-phenoxy)- butylsulfanyl]-2-methyl-phenyl}-propionic acid |
| 68 | 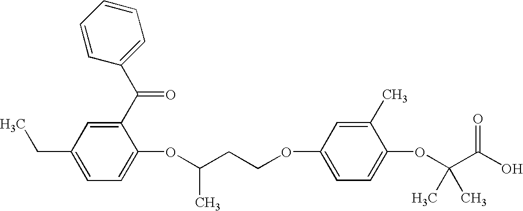 | 2-{4-[3-(2-Benzoyl-4-ethyl-phenoxy)- butoxy]-2-methyl-phenoxy}-2-methyl- propionic acid |
| 69 | 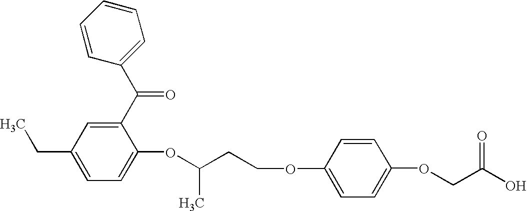 | {4-[3-(2-Benzoyl-4-ethyl-phenoxy)- butoxy]-phenoxy}-acetic acid |
| 70 | 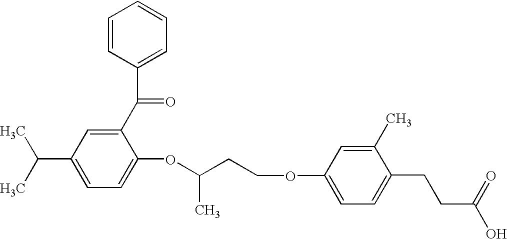 | 3-{4-[3-(2-Benzoyl-4-isopropyl-phenoxy)- butoxy]-2-methyl-phenyl}-propionic acid |
| 71 | 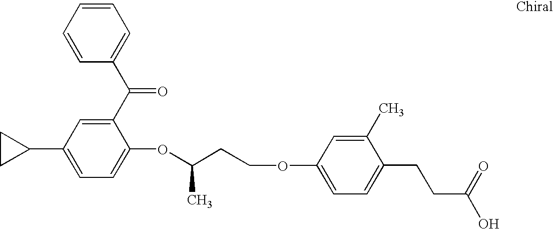 | 3-{4-[3-(2-Benzoyl-4-cyclopropyl-phenoxy)- butoxy]-2-methyl-phenyl}-propionic acid |
| 72 | 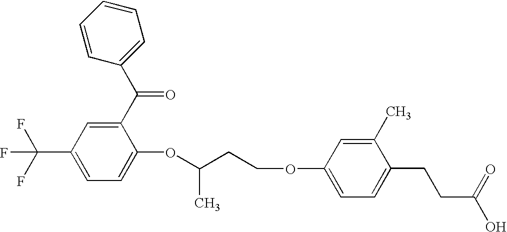 | 3-{4-[3-(2-Benzoyl-4-trifluoromethyl- phenoxy)-butoxy]-2-methyl-phenyl}- propionic acid |
| 73 | 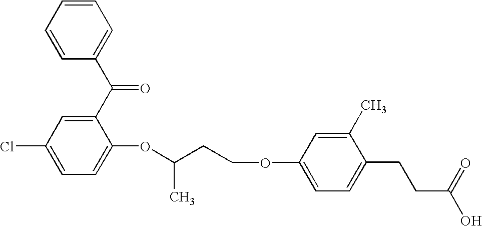 | 3-{4-[3-(2-Benzoyl-4-chloro-phenoxy)- butoxy]-2-methyl-phenyl}-propionic acid |
| 74 | 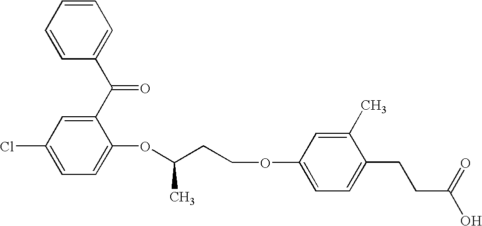 | 3-{4-[3-(2-Benzoyl-4-chloro-phenoxy)- butoxy]-2-methyl-phenyl}-propionic acid |
| 75 | 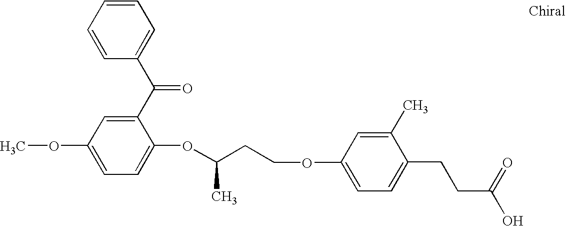 | 3-{4-[3-(2-Benzoyl-4-methoxy-phenoxy)- butoxy]-2-methyl-phenyl}-propionic acid |
| 76 | 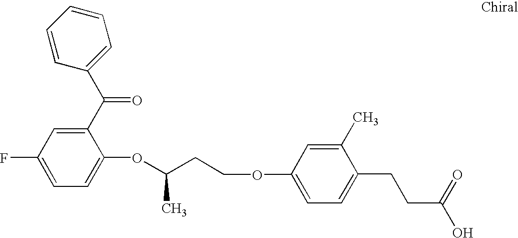 | 3-{4-[3-(2-Benzoyl-4-fluoro-phenoxy)- butoxy]-2-methyl-phenyl}-propionic acid |
| 77 | 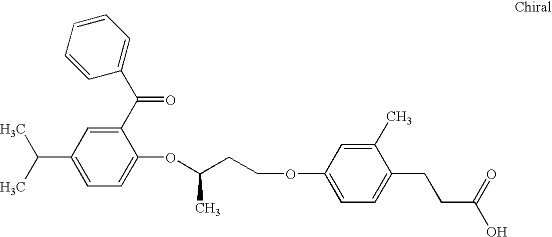 | 3-{4-[3-(2-Benzoyl-4-isopropyl-phenoxy)- butoxy]-2-methyl-phenyl}-propionic acid |
| 78 | 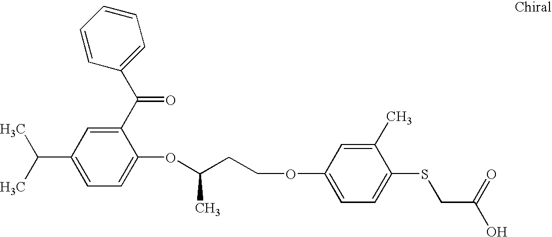 | {4-[3-(2-Benzoyl-4-isopropyl-phenoxy)- butoxy]-2-methyl-phenylsulfanyl}-acetic acid |
| 79 | 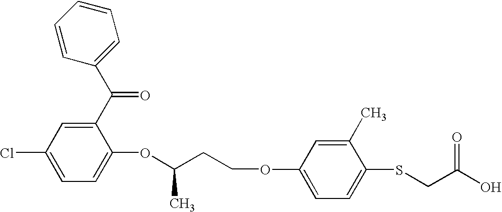 | {4-[3-(2-Benzoyl-4-chloro-phenoxy)- butoxy]-2-methyl-phenylsulfanyl}-acetic acid |
| 80 | 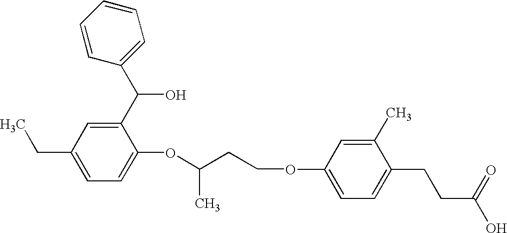 | 3-(4-{3-[4-Ethyl-2-(hydroxy-phenyl-methyl)- phenoxy]-butoxy}-2-methyl-phenyl)- propionic acid |
| 81 | 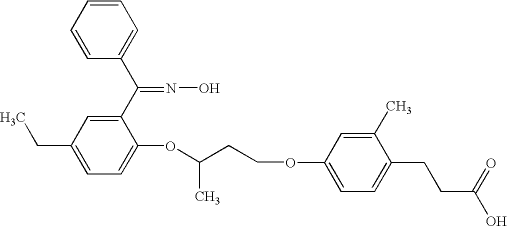 | 3-(4-{3-[4-Ethyl-2-(hydroxyimino-phenyl- methyl)-phenoxy]-butoxy}-2-methyl- phenyl)-propionic acid |
| 82 | 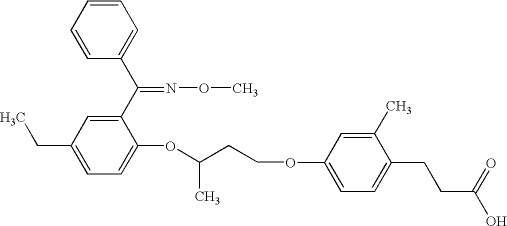 | 3-(4-{3-[4-Ethyl-2-(methoxyimino-phenyl- methyl)-phenoxy]-butoxy}-2-methyl- phenyl)-propionic acid |
| 83 | 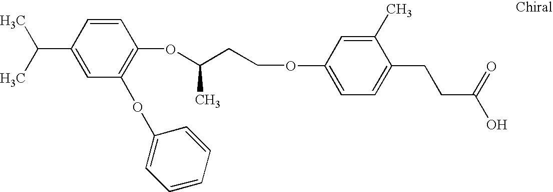 | 3-{4-[3-(4-Isopropyl-2-phenoxy-phenoxy)- butoxy]-2-methyl-phenyl}-propionic acid |
| 84 | 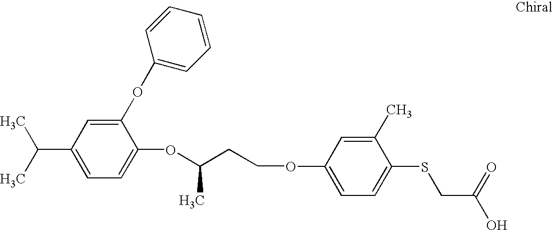 | {4-[3-(4-Isopropyl-2-phenoxy-phenoxy)- butoxy]-2-methyl-phenylsulfanyl}-acetic acid |
| 85 | 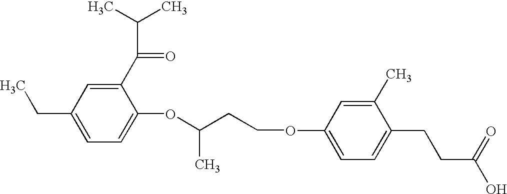 | 3-{4-[3-(4-Ethyl-2-isobutyryl-phenoxy)- butoxy]-2-methyl-phenyl}-propionic acid |
| 86 | 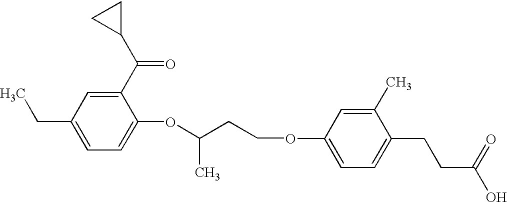 | 3-{4-[3-(2-Cyclopropanecarbonyl-4-ethyl- phenoxy)-butoxy]-2-methyl-phenyl}- propionic acid |
| 87 | 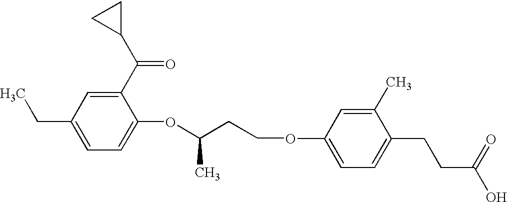 | 3-{4-[3-(2-Cyclopropanecarbonyl-4-ethyl- phenoxy)-butoxy]-2-methyl-phenyl}- propionic acid |
| 88 | 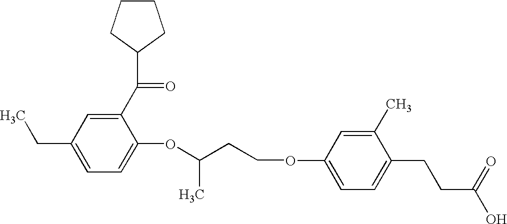 | 3-{4-[3-(2-Cyclopentanecarbonyl-4-ethyl- phenoxy)-butoxy]-2-methyl-phenyl}- propionic acid |
| 89 | 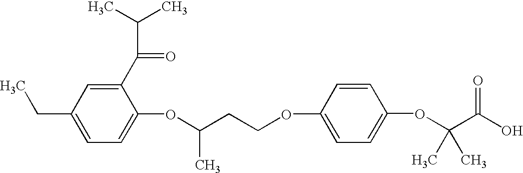 | 2-{4-[3-(4-Ethyl-2-isobutyryl-phenoxy)- butoxy]-phenoxy}-2-methyl-propionic acid |
| 90 | 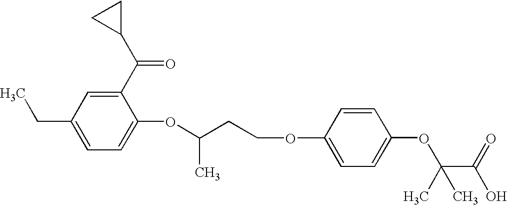 | 2-{4-[3-(2-Cyclopropanecarbonyl-4-ethyl- phenoxy)-butoxy]-phenoxy}-2-methyl- propionic acid |
| 91 | 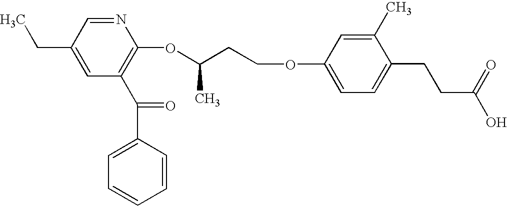 | 3-{4-[3-(3-Benzoyl-5-ethyl-pyridin-2-yloxy)- butoxy]-2-methyl-phenyl)-propionic acid |
| 92 | 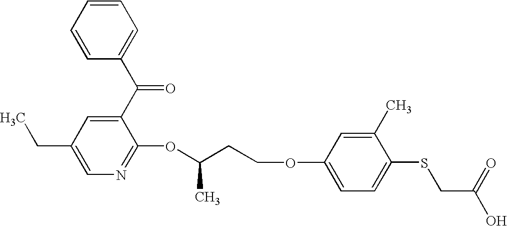 | {4-[3-(3-Benzoyl-5-ethyl-pyridin-2-yloxy)- butoxy]-2-methyl-phenylsulfanyl}-acetic acid |
| 93 | 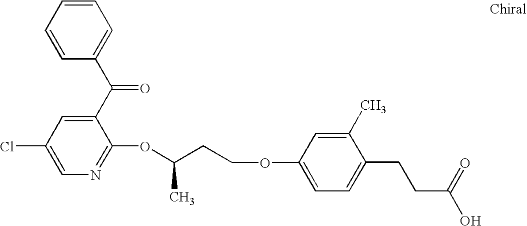 | 3-{4-[3-(3-Benzoyl-5-chloro-pyridin-2- yloxy)-butoxy]-2-methyl-phenyl}-propionic acid |
| 94 | 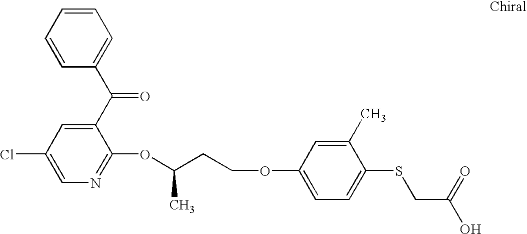 | {4-[3-(3-Benzoyl-5-chloro-pyridin-2-yloxy)- butoxy]-2-methyl-phenylsulfanyl)-acetic acid |
| 95 | 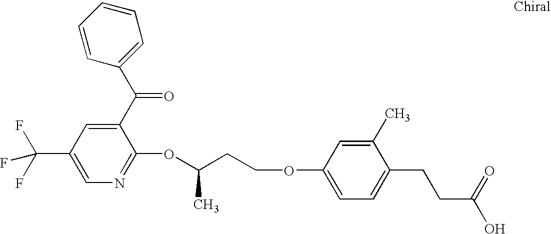 | 3-{4-[3-(3-Benzoyl-5-trifluoromethyl- pyridin-2-yloxy)-butoxy]-2-methyl-phenyl}- propionic acid |
| 96 | 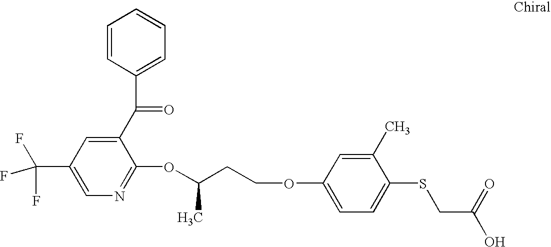 | {4-[3-(3-Benzoyl-5-trifluoromethyl- pyridin-2-yloxy)-butoxy]-2-methyl- phenylsulfanyl}-acetic acid |
| 97 | 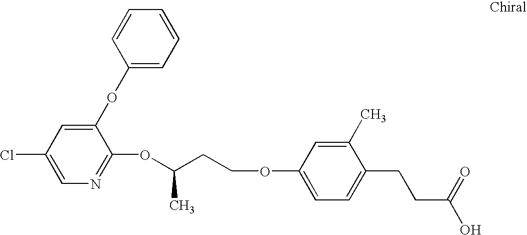 | 3-{4-[3-(5-Chloro-3-phenoxy-pyridin- 2-yloxy)-butoxy]-2-methyl-phenyl}- propionic acid |
| 98 | 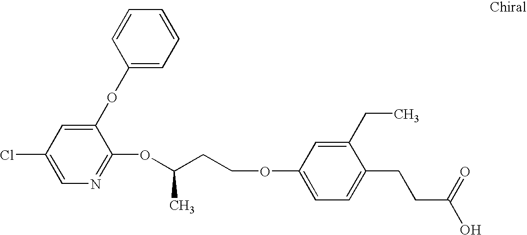 | 3-{4-[3-(5-Chloro-3-phenoxy-pyridin- 2-yloxy)-butoxy]-2-ethyl-phenyl}- propionic acid |
| 99 | 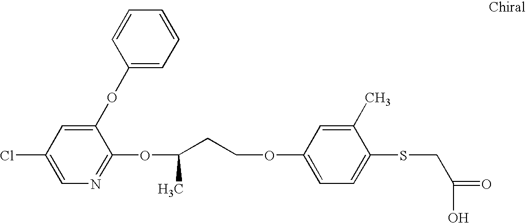 | {4-[3-(5-Chloro-3-phenoxy-pyridin-2-yloxy)- butoxy]-2-methyl-phenylsulfanyl}-acetic acid |
| 100 | 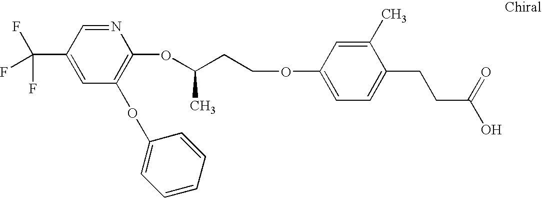 | 3-{2-Methyl-4-[3-(3-phenoxy-5- trifluoromethyl-pyridin-2-yloxy)-butoxy]- phenyl}-propionic acid |
| 101 | 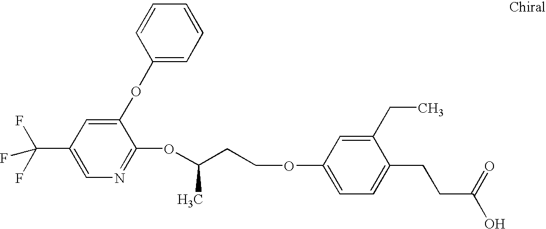 | 3-{2-Ethyl-4-[3-(3-phenoxy-5- trifluoromethyl-pyridin-2-yloxy)-butoxy]- phenyl}-propionic acid |
| 102 | 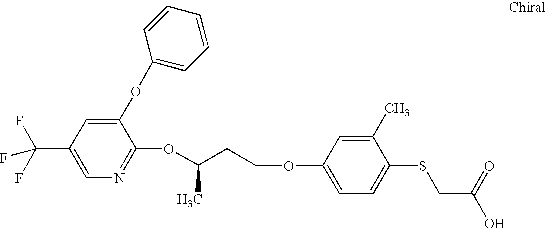 | 3-{2-Ethyl-4-[3-(3-phenoxy-5- trifluoromethyl-pyridin-2-yloxy)-butoxy]- phenyl}-propionic acid |
| 103 | 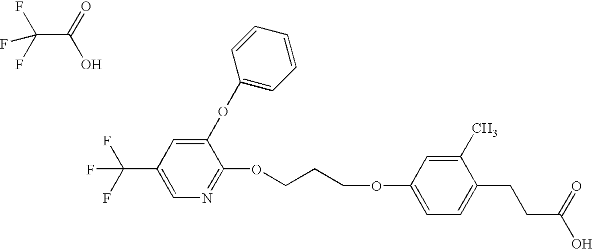 | 3-{2-Methyl-4-[3-(3-phenoxy-5- trifluoromethyl-pyridin-2-yloxy)-propoxy]- phenyl}-propionic acid (trifluoroacetic acid salt) |
| 104 | 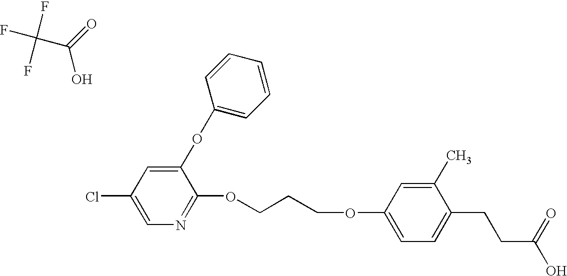 | 3-{4-[3-(5-Chloro-3-phenoxy-pyridin-2- yloxy)-propoxy]-2-methyl-phenyl }- propionic acid |
| 105 | 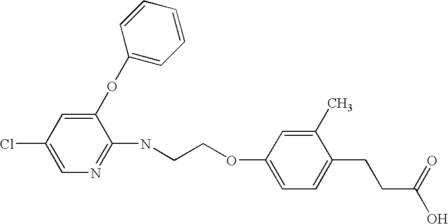 | 3-{4-[2-(5-Chloro-3-phenoxy-pyridin-2- ylamino)-ethoxy]-2-methyl-phenyl}- propionic acid |
| 106 | 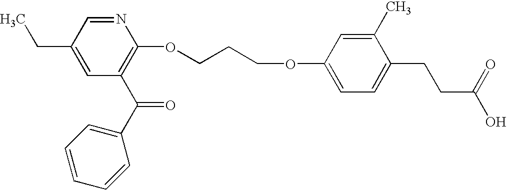 | 3-{4-[3-(3-Benzoyl-5-ethyl-pyridin-2-yloxy)- propoxy]-2-methyl-phenyl}-propionic acid |
| 107 | 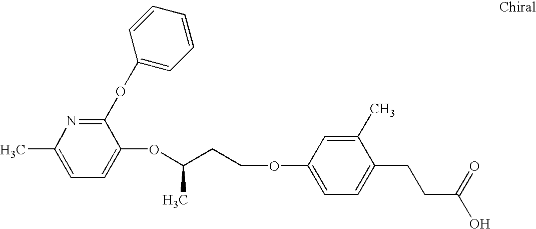 | 3-{2-Methyl-4-[3-(6-methyl-2-phenoxy- pyridin-3-yloxy)-butoxy]-phenyl}-propionic acid |
| 108 | 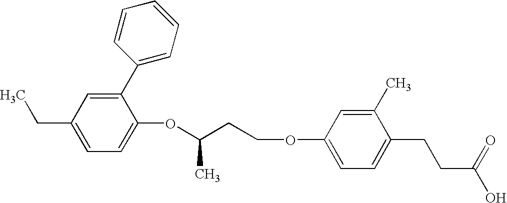 | 3-{4-[3-(5-Ethyl-biphenyl-2-yloxy)-butoxy]- 2-methyl-phenyl}-propionic acid |
| 109 | 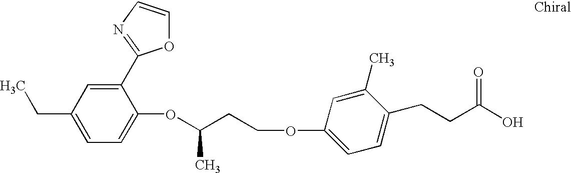 | 3-{4-[3-(4-Ethyl-2-oxazol-2-yl-phenoxy)- butoxy]-2-methyl-phenyl}-propionic acid |
| 110 | 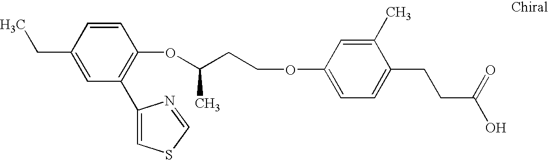 | 3-{4-[3-(4-Ethyl-2-thiazol-4-yl-phenoxy)- butoxy]-2-methyl-phenyl}-propionic acid |
| 111 | 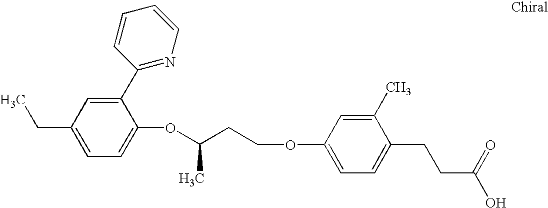 | 3-{4-[3-(4-Ethyl-2-pyridin-2-yl-phenoxy)- butoxy]-2-methyl-phenyl}-propionic acid |
| 112 | 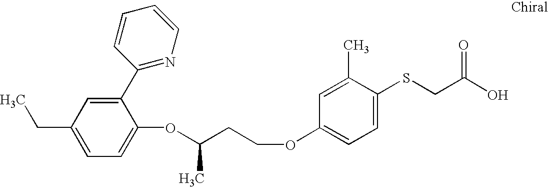 | {4-[3-(4-Ethyl-2-pyridin-2-yl-phenoxy)- butoxy]-2-methyl-phenylsulfanyl}-acetic acid |
| 113 | 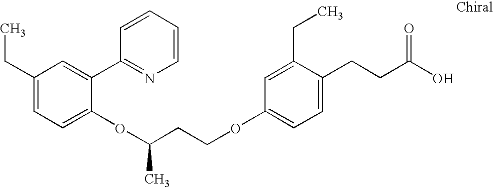 | 3-{2-Ethyl-4-[3-(4-ethyl-2-pyridin-2-yl- phenoxy)-butoxy]-phenyl}-propionic acid |
| 114 | 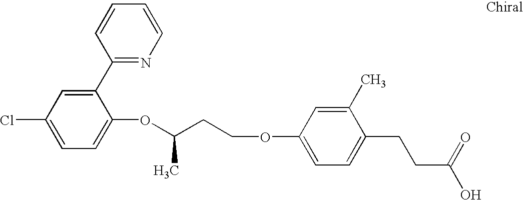 | 3-{4-[3-(4-Chloro-2-pyridin-2-yl-phenoxy)- butoxy]-2-methyl-phenyl}-propionic acid |
| 115 | 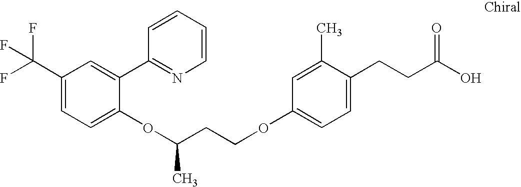 | 3-{2-Methyl-4-[3-(2-pyridin-2-yl-4- trifluoromethyl-phenoxy)-butoxy]-phenyl}- propionic acid |
| 116 | 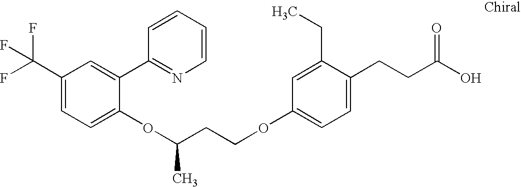 | 3-{2-Ethyl-4-[3-(2-pyridin-2-yl-4- trifluoromethyl-phenoxy)-butoxy]-phenyl}- propionic acid |
| 117 | 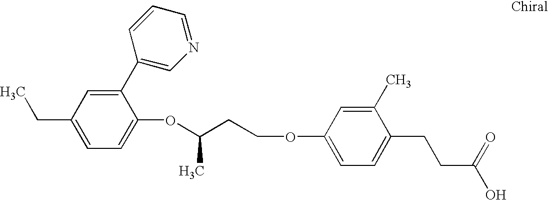 | 3-{4-[3-(4-Ethyl-2-pyridin-3-yl-phenoxy)- butoxy]-2-methyl-phenyl}-propionic acid |
| 118 | 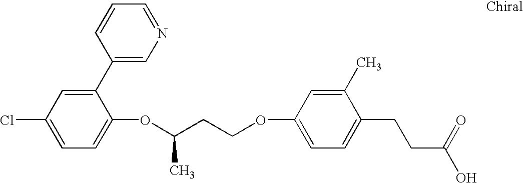 | 3-{4-[3-(4-Chloro-2-pyridin-3-yl-phenoxy)- butoxy]-2-methyl-phenyl}-propionic acid |
| 119 | 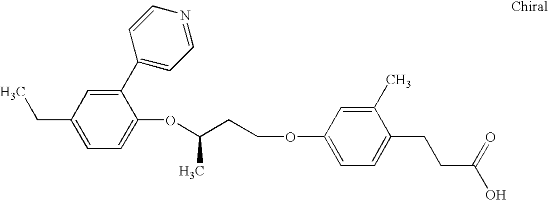 | 3-{4-[3 -(4-Ethyl-2-pyridin-4-yl-phenoxy)- butoxy]-2-methyl-phenyl}-propionic acid |
| 120 | 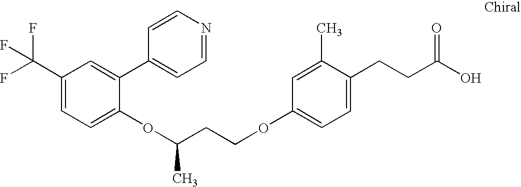 | 3-{2-Methyl-4-[3-(2-pyridin-4-yl-4- trifluoromethyl-phenoxy)-butoxy]-phenyl}- propionic acid |
| 121 | 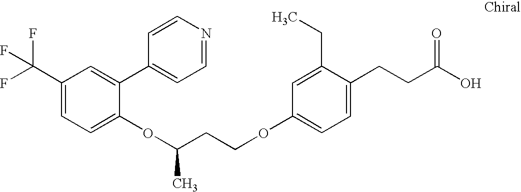 | 3-{2-Ethyl-4-[3-(2-pyridin-4-yl-4- trifluoromethyl-phenoxy)-butoxy]-phenyl}- propionic acid |
| 122 | 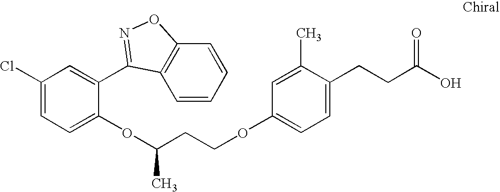 | 3-{4-[3-(2-Benzo[d]isoxazol-3-yl-4-chloro- phenoxy)-butoxy]-2-methyl-phenyl}- propionic acid |
| 123 | 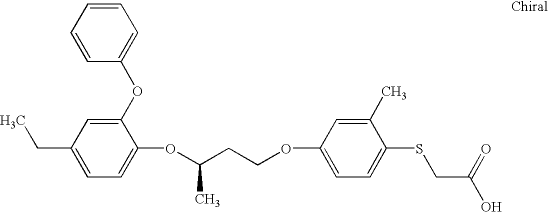 | (R)-{4-[3-(4-ethyl-2-phenoxy-phenoxy)- butoxy]-2-methyl-phenylsulfanyl}-acetic acid |
| 124 | 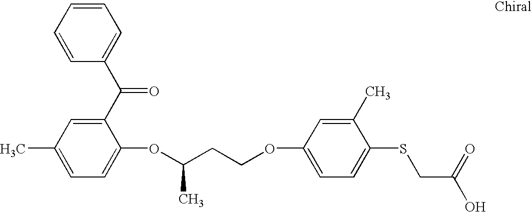 | (R)-{4-[3-(2-benzoyl-4-methyl-phenoxy)- butoxy]-2-methyl-phenylsulfanyl}-acetic acid |
| 125 | 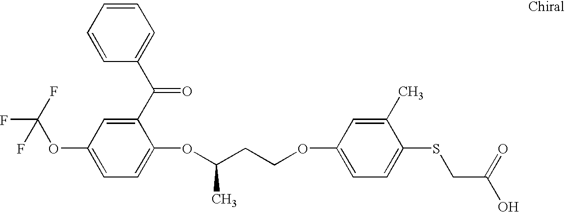 | (R)-{4-[3-(2-benzoyl-4-trifluoromethoxy- phenoxy)-butoxy]-2-methyl-phenylsulfanyl}- acetic acid |
| 126 | 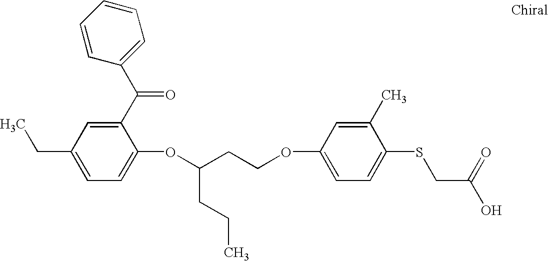 | {4-[3-(2-benzoyl-4-ethyl-phenoxy)- hexyloxy]-2-methyl-phenylsulfanyl}-acetic acid |
| 127 | 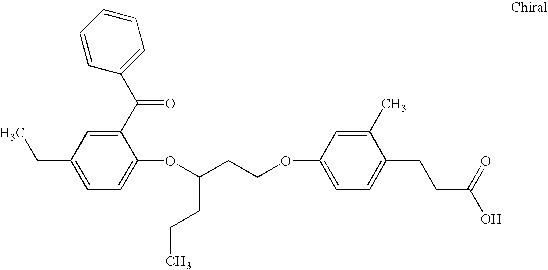 | 3-{4-[3-(2-benzoyl-4-ethyl-phenoxy)- hexyloxy]-2-methyl-phenyl}-propionic acid |
| 128 | 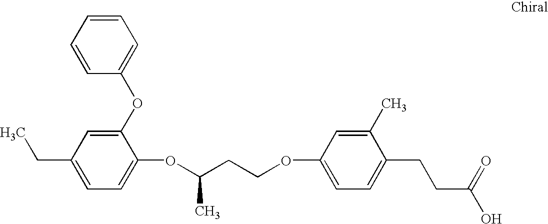 | (R)-3-{4-[3-(4-ethyl-2-phenoxy-phenoxy)- butoxy]-2-methyl-phenyl}-propionic acid |
| 129 | 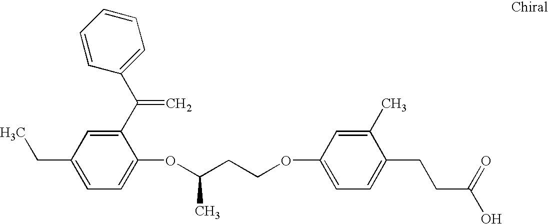 | (R)-3-(4-{3-[4-ethyl-2-(1-phenyl-vinyl)- phenoxy]-butoxy}-2-methyl-phenyl)- propionic acid |
| 130 | 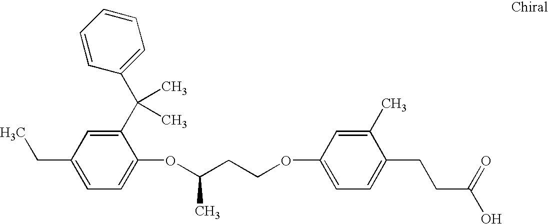 | (R)-3-(4-{3-[4-ethyl-2-(1-methyl-1-phenyl- ethyl)-phenoxy]-butoxy}-2-methyl-phenyl)- propionic acid |
| 131 | 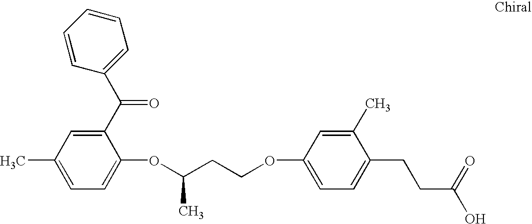 | (R)-3-{4-[3-(2-benzoyl-4-methyl-phenoxy)- butoxy]-2-methyl-phenyl}-propionic acid |
| 132 | 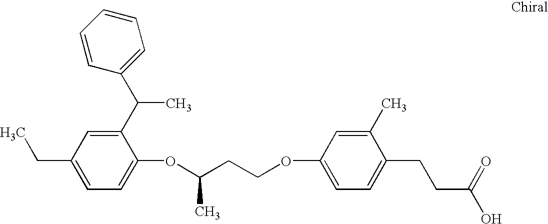 | (R)-3-(4-{3-[4-ethyl-2-(1-phenyl-ethyl)- phenoxy]butoxy}-2-methyl-phenyl)- propionic acid |
| 133 | 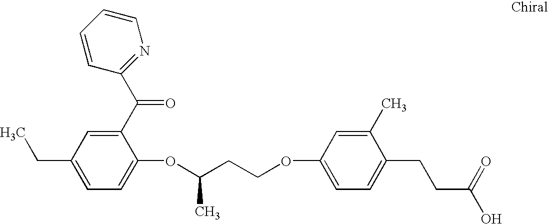 | (R)-3-(4-{3-[4-ethyl-2-(pyridine-2-carbonyl)- phenoxy]-butoxy}-2-methyl-phenyl)- propionic acid |
| 134 | 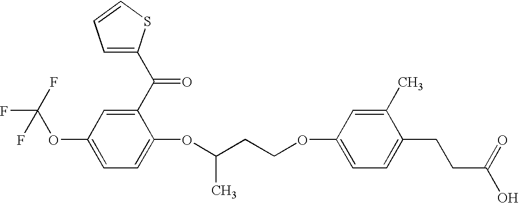 | 3-(2-methyl-4-{3-[2-(thiophene-2-carbonyl)- 4-trifluoromethoxy-phenoxy]-butoxy}- phenyl)-propionic acid |
| 135 | 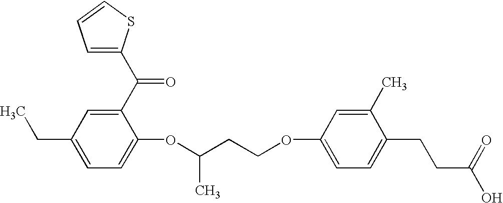 | 3-(4-{3-[4-ethyl-2-(thiophene-2-carbonyl)- phenoxy]-butoxy}-2-methyl-phenyl)- propionic acid |
| 136 | 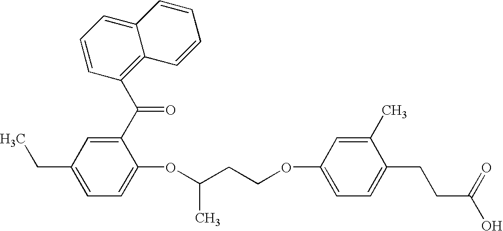 | 3-(4-{3-[4-ethyl-2-(naphthalene-1-carbonyl)- phenoxy]-butoxy}-2-methyl-phenyl)- propionic acid |
| 137 | 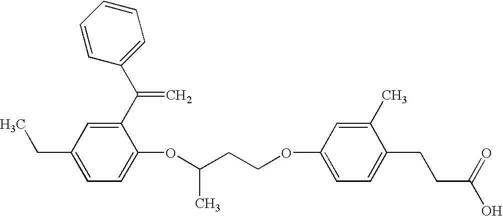 | 3-(4-{3-[4-ethyl-2-(1-phenyl-vinyl)- phenoxy]-butoxy}-2-methyl-phenyl)- propionic acid |
| 138 | 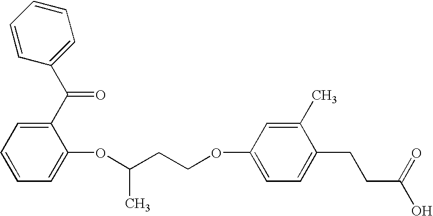 | 3-{4-[3-(2-benzoyl-phenoxy)-butoxy]-2- methyl-phenyl}-propionic acid |
| 139 | 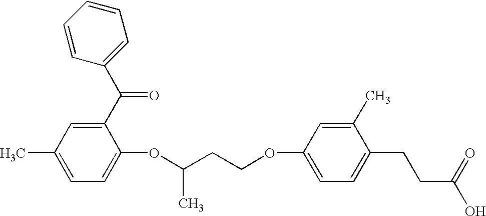 | 3-{4-[3-(2-benzoyl-4-methyl-phenoxy)- butoxy]-2-methyl-phenyl}-propionic acid |
| 140 | 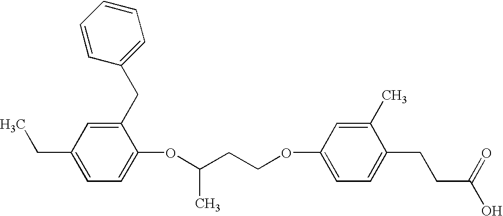 | 3-{4-[3-(2-benzyl-4-ethyl-phenoxy)- butoxy]-2-methyl-phenyl}-propionic acid |
| 141 | 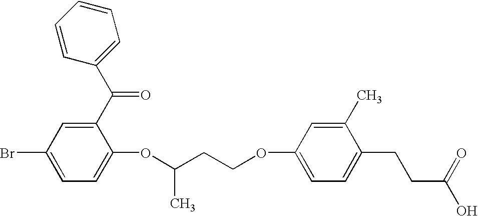 | 3-{4-[3-(2-benzoyl-4-bromo-phenoxy)- butoxy]-2-methyl-phenyl}-propionic acid |
| 142 | 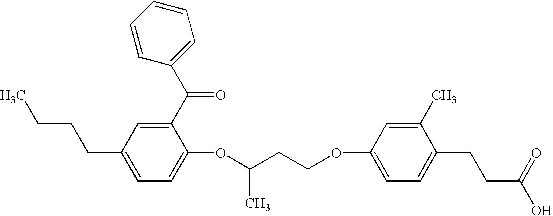 | 3-{4-[3-(2-benzoyl-4-butyl-phenoxy)- butoxy]-2-methyl-phenyl}-propionic acid |
| 143 | 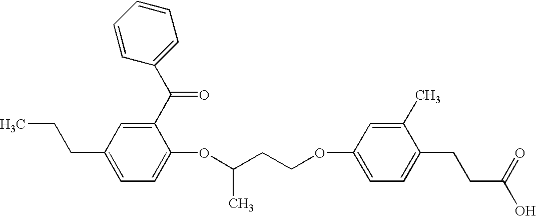 | 3-{4-[3-(2-benzoyl-4-propyl-phenoxy)- butoxy]-2-methyl-phenyl}-propionic acid |
| 144 | 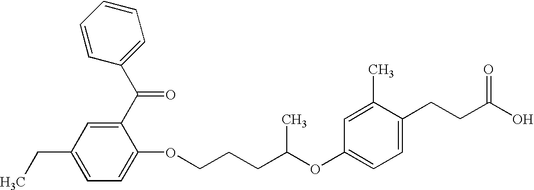 | 3-{4-[4-(2-benzoyl-4-ethyl-phenoxy)-1- methyl-butoxy]-2-methyl-phenyl}-propionic acid |
| 145 | 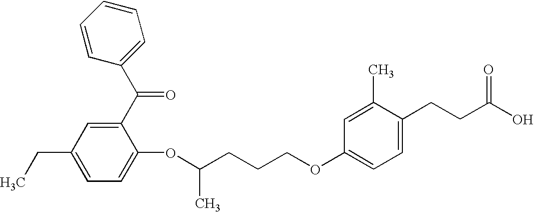 | 3-{4-[4-(2-benzoyl-4-ethyl-phenoxy)- pentyloxy]-2-methyl-phenyl}-propionic acid |
| 146 | 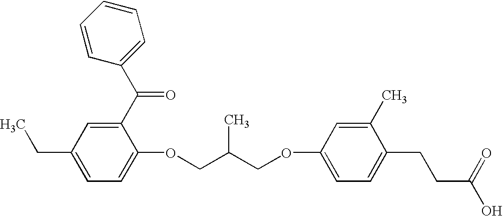 | 3-{4-[3-(2-benzoyl-4-ethyl-phenoxy)-2- methyl-propoxy]-2-methyl-phenyl}- propionic acid |
| 147 | 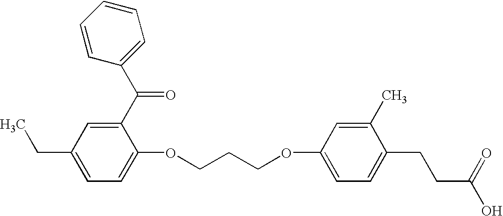 | 3-{4-[3-(2-benzoyl-4-ethyl-phenoxy)- propoxy]-2-methyl-phenyl}-propionic acid |
| 148 | 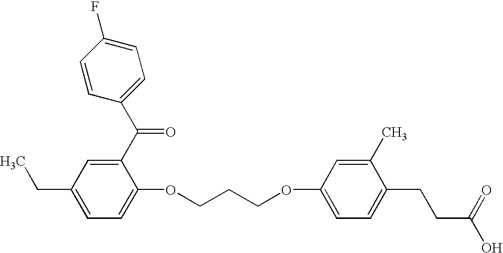 | 3-(4-{3-[4-ethyl-2-(4-fluoro-benzoyl)- phenoxy]-propoxy}-2-methyl-phenyl)- propionic acid |
| 149 | 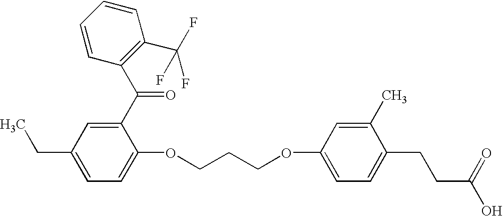 | 3-(4-{3-[4-ethyl-2-(2-trifluoromethyl- benzoyl)-phenoxy]-propoxy}-2-methyl- phenyl)-propionic acid |
| 150 | 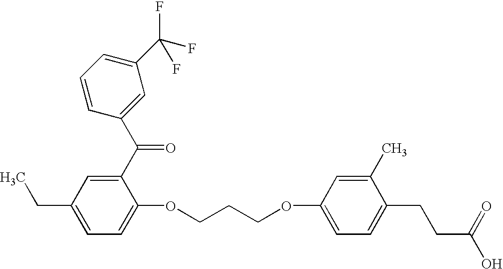 | 3-(4-{3-[4-ethyl-2-(3-trifluoromethyl- benzoyl)-phenoxy]-propoxy}-2-methyl- phenyl)-propionic acid |
| 151 | 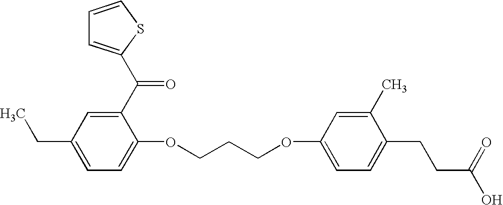 | 3-(4-{3-[4-ethyl-2-(thiophene-2-carbonyl)- phenoxy]-propoxy}-2-methyl-phenyl)- propionic acid |
| 152 | 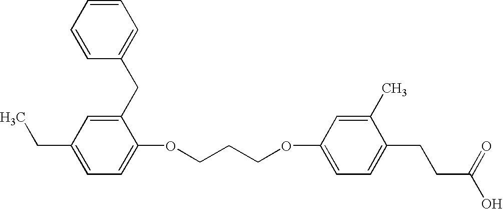 | 3-{4-[3-(2-benzyl-4-ethyl-phenoxy)- propoxy]-2-methyl-phenyl}-propionic acid |
| 153 | 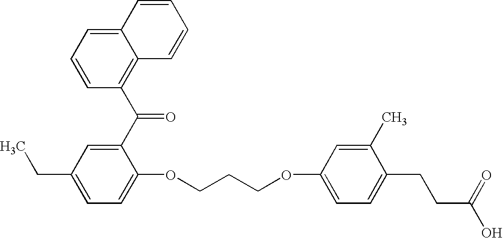 | 3-(4-{3-[4-ethyl-2-(naphthalene-1- carbonyl)-phenoxy]-propoxy}-2-methyl- phenyl)-propionic acid |
| 154 | 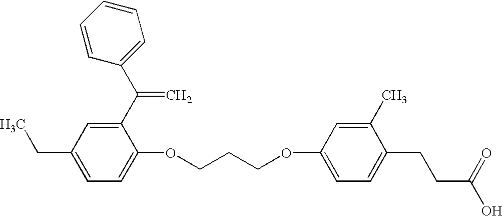 | 3-(4-{3-[4-ethyl-2-(1-phenyl-vinyl)- phenoxy]-propoxy}-2-methyl-phenyl)- propionic acid |
| 155 | 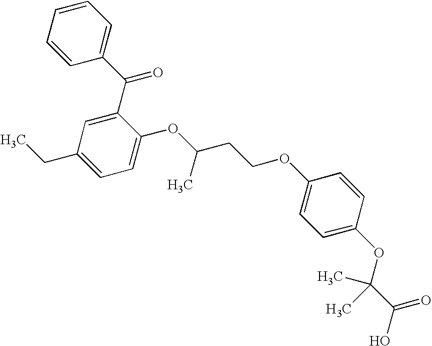 | 2-{4-[3-(2-benzoyl-4-ethyl-phenoxy)- butoxy]-phenoxy}-2-methyl-propionic acid |
| 156 | 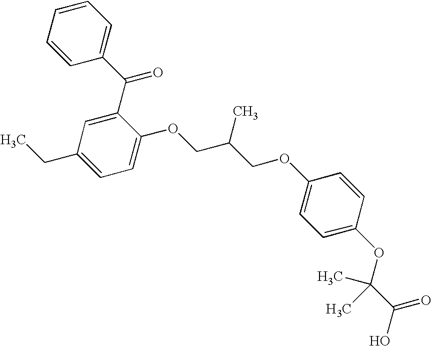 | 2-{4-[3-(2-benzoyl-4-ethyl-phenoxy)-2- methyl-propoxy]-phenoxy}-2-methyl- propionic acid |
| 157 | 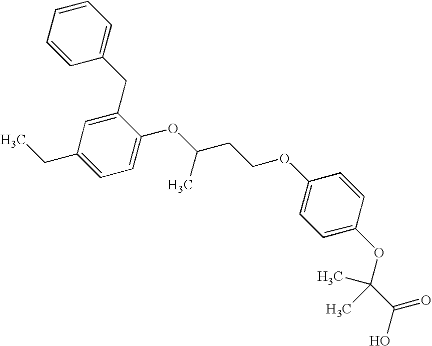 | 2-{4-[3-(2-benzyl-4-ethyl-phenoxy)- butoxy]-phenoxy}-2-methyl-propionic acid |
| 158 | 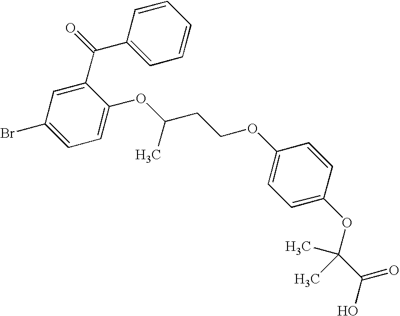 | 2-{4-[3-(2-benzoyl-4-bromo-phenoxy)- butoxy]-phenoxy}-2-methyl-propionic acid |
| 159 | 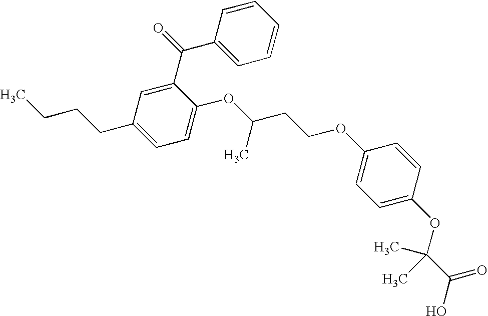 | 2-{4-[3-(2-benzoyl-4-butyl-phenoxy)- butoxy]-phenoxy}-2-methyl-propionic acid |
| 160 | 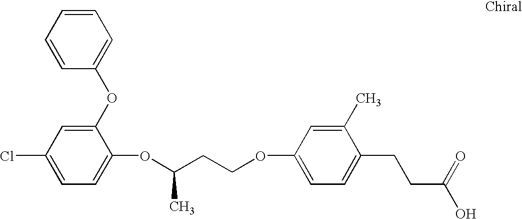 | (R)-3-{4-[3-(4-chloro-2-phenoxy- phenoxy)-butoxy]-2-methyl-phenyl}- propionic acid |
| 161 | 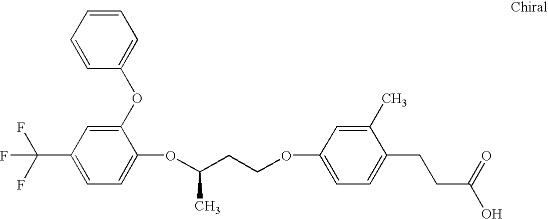 | (R)-3-{2-methyl-4-[3-(2-phenoxy-4- trifluoromethyl-phenoxy)-butoxy]-phenyl}- propionic acid |
| 162 | 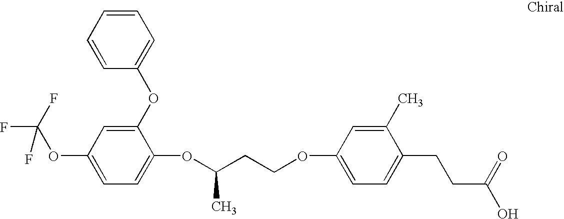 | (R)-3-{2-methyl-4-[3-(2-phenoxy-4- trifluoromethoxy-phenoxy)-butoxy]-phenyl}- propionic acid |
| 163 | 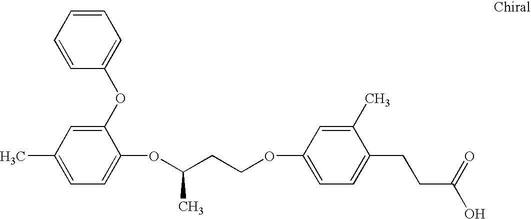 | (R)-3-{2-methyl-4-[3-{4-methyl-2-phenoxy- phenoxy)-butoxy]-phenyl}-propionic acid |
| 164 | 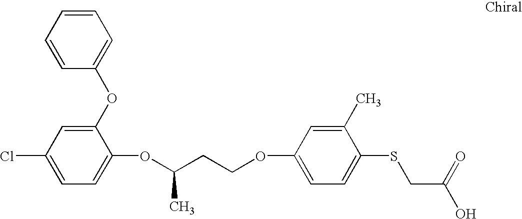 | (R)-{4-[3-(4-chloro-2-phenoxy-phenoxy)- butoxy]-2-methyl-phenylsulfanyl}-acetic acid |
| 165 | 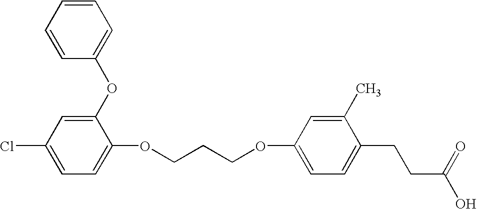 | 3-{4-[3-(4-chloro-2-phenoxy-phenoxy)- propoxy]-2-methyl-phenyl}-propionic acid |
| 166 | 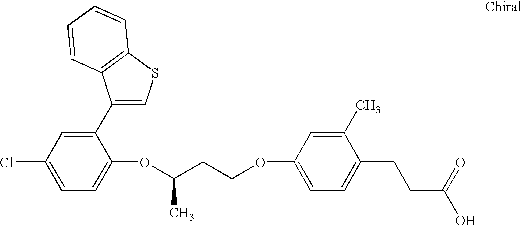 | (R)-3-{4-[3-(2-benzo[b]thiophen-3-yl-4- chloro-phenoxy)-butoxy]-2-methyl-phenyl}- propionic acid |
| 167 | 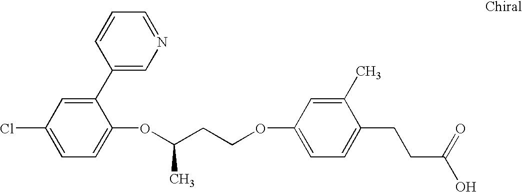 | (R) 3-{4-[3-(4-chloro-2-pyridin-3-yl- phenoxy)-butoxy]-2-methyl-phenyl}- propionic acid |
| 168 | 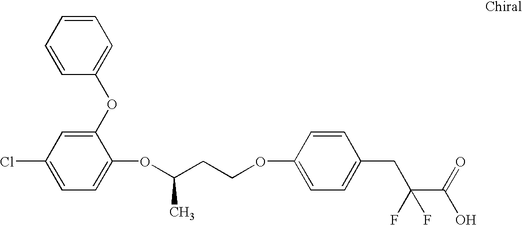 | (R)-3-{4-[3-(4-chloro-2-phenoxy-phenoxy)- butoxy]-phenyl}-2,2-difluoro-propionic acid |
| 169 | 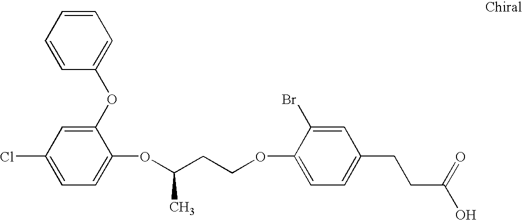 | %)(R)-3-{3-bromo-4-[3-(4-chloro-2- phenoxy-phenoxy)-butoxy]-phenyl}- propionic acid |
| 170 | 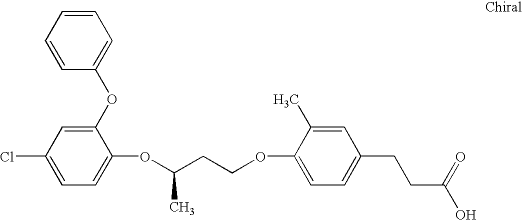 | (R)-3-{4-[3-(4-chloro-2-phenoxy-phenoxy)- butoxy]-3-methyl-phenyl}-propionic acid |
| 171 | 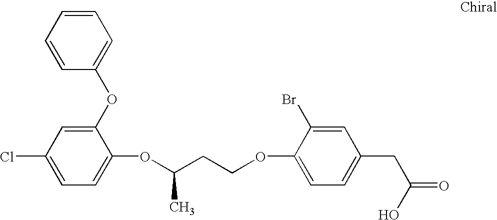 | (R)-{3-bromo-4-[3-(4-chloro-2-phenoxy- phenoxy)-butoxy]-phenyl}-acetic acid |
| 172 | 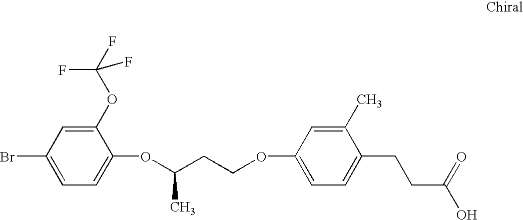 | (R)-3-{4-[3-(4-bromo-2-trifluoromethoxy- phenoxy)-butoxy]-2-methyl-phenyl}- propionic acid |
| 173 | 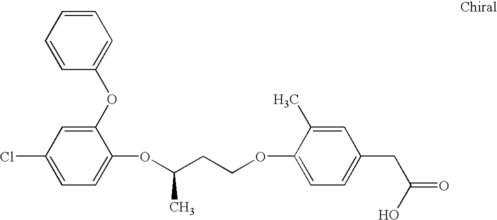 | (R)-{4-[3-(4-chloro-2-phenoxy-phenoxy)- butoxy]-3-methyl-phenyl}-acetic acid |
| 174 | 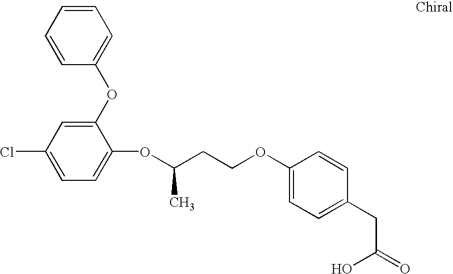 | (R)-{4-[3-(4-chloro-2-phenoxy-phenoxy)- butoxy]-phenyl}-acetic acid |
| 175 | 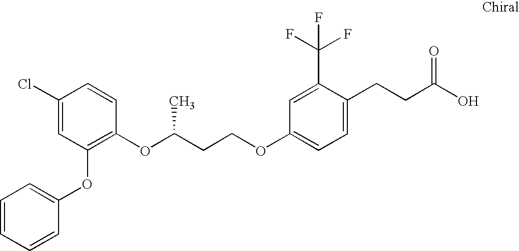 | (R)-3-{4-[3-(4-chloro-2-phenoxy-phenoxy)- butoxy]-2-trifluoromethyl-phenyl}-propionic acid |
| 176 | 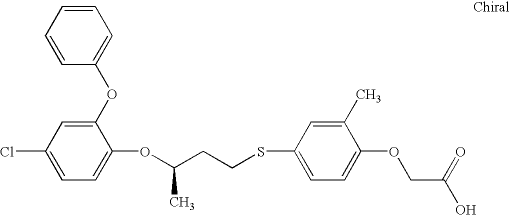 | (R)-{4-[3-(4-chloro-2-phenoxy-phenoxy)- butylsulfanyl]-2-methyl-phenoxy}-acetic acid |
| 177 | 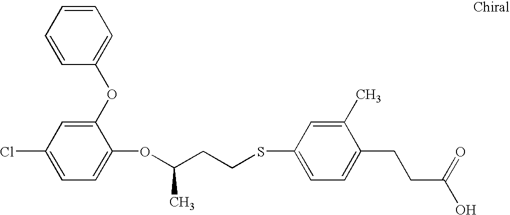 | (R)-3-{4-[3-(4-chloro-2-phenoxy-phenoxy)- butylsulfanyl]-2-methyl-phenyl}-propionic acid |
| 178 | 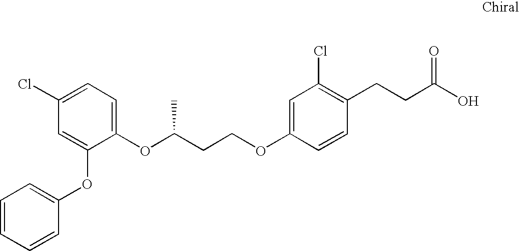 | (R)-3-{2-Chloro-4-[3-(4-chloro-2-phenoxy- phenoxy)-butoxy]-phenyl}-propionic acid |
| 179 | 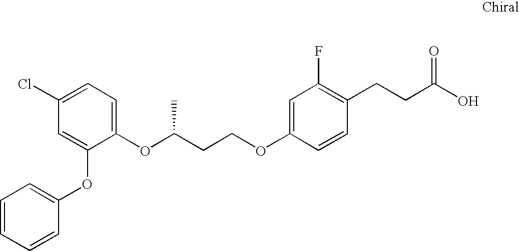 | (R)-3-{4-[3-(4-Chloro-2-phenoxy-phenoxy)- butoxy]-2-fluoro-phenyl}-propionic acid |
| 180 | 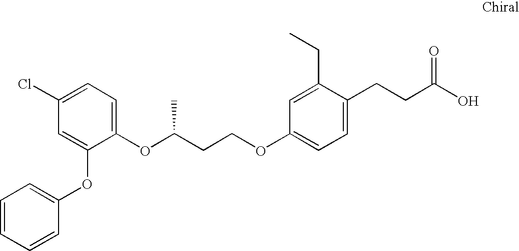 | (R)-3-{4-[3-(4-Chloro-2-phenoxy-phenoxy)- butoxy]-2-ethyl-phenyl}-propionic acid |
| 181 | 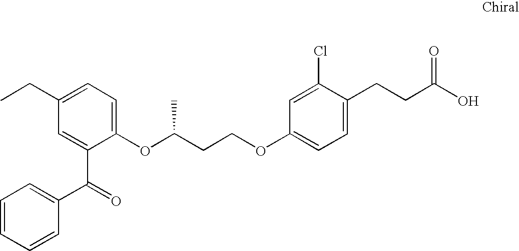 | (R)-3-{4-[3-(2-Benzoyl-4-ethyl-phenoxy)- butoxy]-2-chloro-phenyl}-propionic acid |
| 182 | 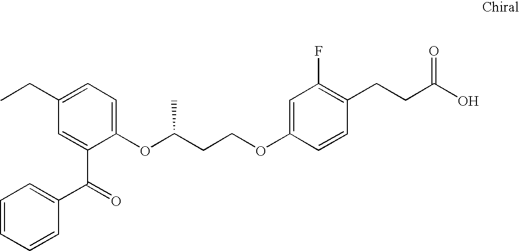 | (R)-3-{4-[3-(2-Benzoyl-4-ethyl-phenoxy)- butoxy]-2-fluoro-phenyl}-propionic acid |
| 183 | 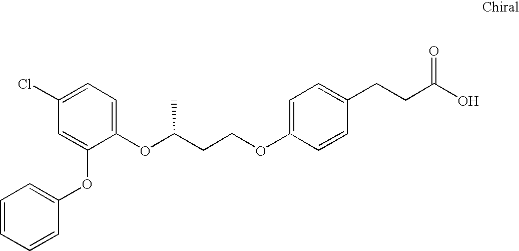 | (R)-3-{4-[3-(4-Chloro-2-phenoxy-phenoxy)- butoxy]-phenyl}-propionic acid |
| 184 | 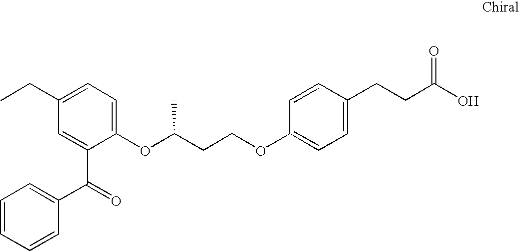 | (R)-3-{4-[3-(2-Benzoyl-4-ethyl-phenoxy)- butoxy]-phenyl}-propionic acid |
| 185 | 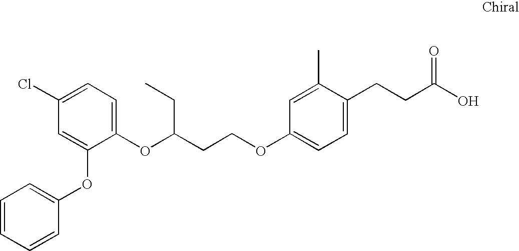 | (R)-3-{4-[3-(4-Chloro-2-phenoxy-phenoxy)- pentyloxy]-2-methyl-phenyl}-propionic acid |
| | Isomer 1 | |
| 186 | 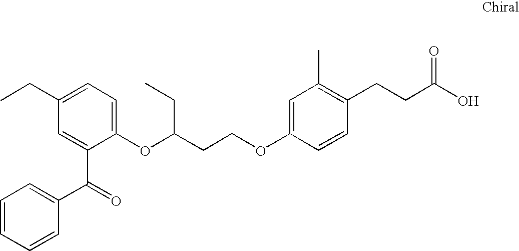 | (R)-3-{4-[3-(2-Benzoyl-4-ethyl-phenoxy)- pentyloxy]-2-methyl-phenyl}-propionic acid |
| | Isomer 1 | |
| 187 | 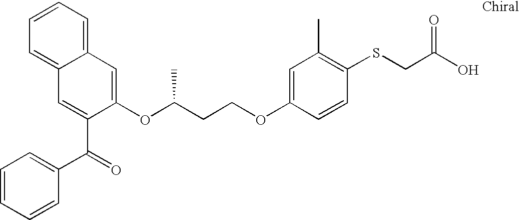 | (R)-{4-[3-(3-Benzoyl-naphthalen-2-yloxy)- butoxy]-2-methyl-phenylsulfanyl}-acetic acid |
| 188 | 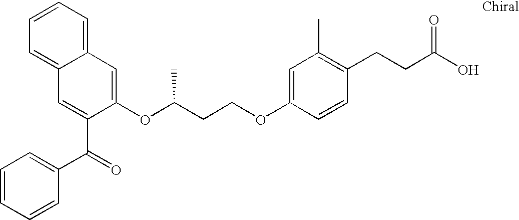 | (R)-3-{4-[3-(3-Benzoyl-naphthalen-2-yloxy)- butoxy]-2-methyl-phenyl}-propionic acid |
| 189 | 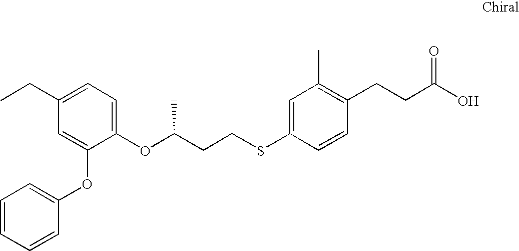 | (R)-3-{4-[3-(4-Ethyl-2-phenoxy-phenoxy)- butylsulfanyl]-2-methyl-phenyl}-propionic acid |
| 190 | 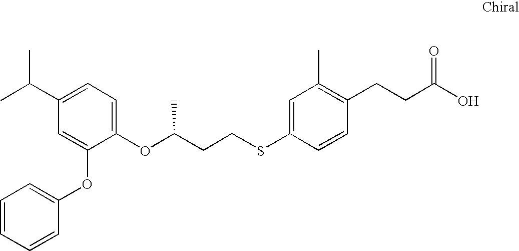 | (R)-3-{4-[3-(4-Isopropyl-2-phenoxy- phenoxy)-butylsulfanyl]-2-methyl-phenyl}- propionic acid |
| 191 | 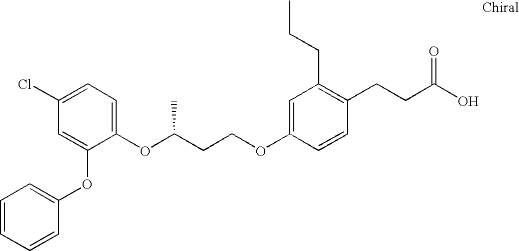 | (R)-3-{4-[3-(4-Chloro-2-phenoxy-phenoxy)- butoxy]-2-propyl-phenyl}-propionic acid |
| 192 | 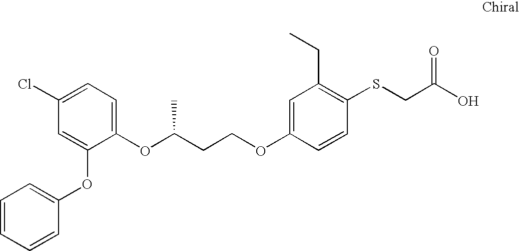 | (R)-{4-[3-(4-Chloro-2-phenoxy-phenoxy)- butoxy]-2-ethyl-phenylsulfanyl}-acetic acid |
| 193 | 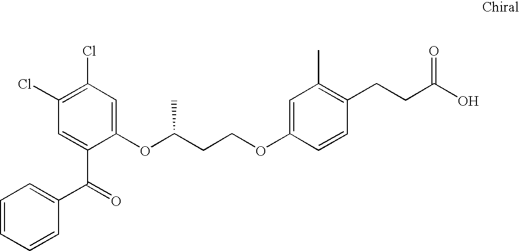 | (R)-3-{4-[3-(2-Benzoyl-4,5-dichloro- phenoxy)-butoxy]-2-methyl-phenyl}- propionic acid |
| 194 | 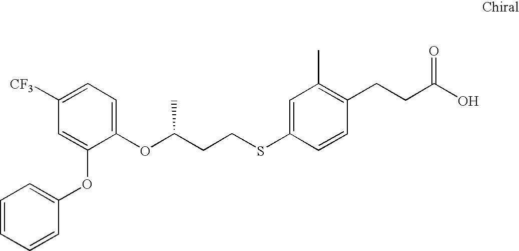 | (R)-3-{2-Methyl-4-[3-(2-phenoxy-4- trifluoromethyl-phenoxy)-butylsulfanyl]- phenyl}-propionic acid |
| 195 | 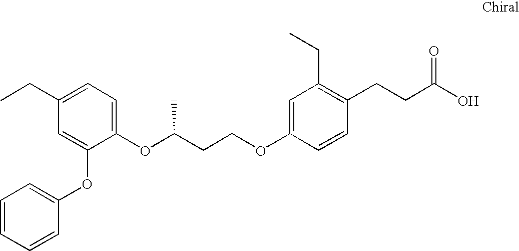 | (R)-3-{2-Ethyl-4-[3-(4-ethyl-2-phenoxy- phenoxy)-butoxy]-phenyl}-propionic acid |
| 196 | 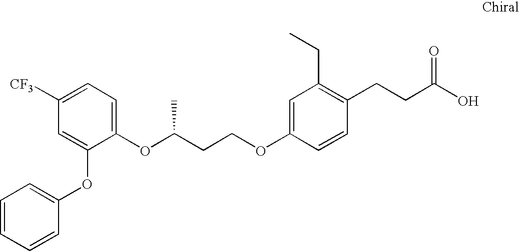 | (R)-3-{2-Ethyl-4-[3-(2-phenoxy-4- trifluoromethyl-phenoxy)-butoxy]-phenyl}- propionic acid |
| 197 | 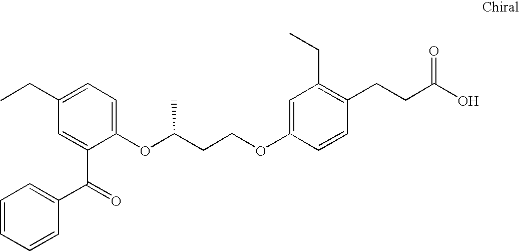 | (R)-3-{4-[3-(2-Benzoyl-4-ethyl-phenoxy)- butoxy]-2-ethyl-phenyl}-propionic acid |
| 198 | 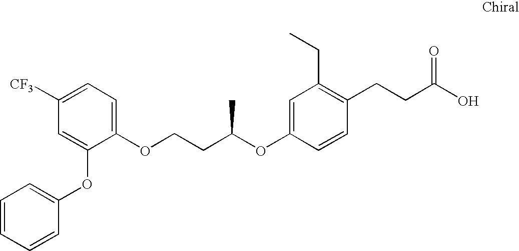 | (R)-3-{2-Ethyl-4-[1-methyl-3-(2-phenoxy-4- trifluoromethyl-phenoxy)-propoxy]-phenyl}- propionic acid |
| 199 | 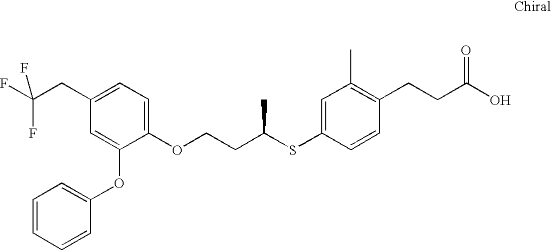 | (R)-3-{2-Methyl-4-[1-methyl-3-(2-phenoxy- 4-trifluoromethoxy-phenoxy)- propylsulfanyl]-phenyl}-propionic acid |
| 200 | 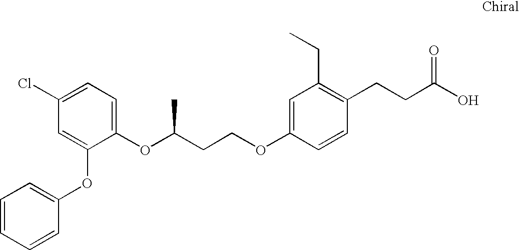 | (S)-3-{4-[3-(4-Chloro-2-phenoxy-phenoxy)- butoxy]-2-ethyl-phenyl}-propionic acid |
| 201 | 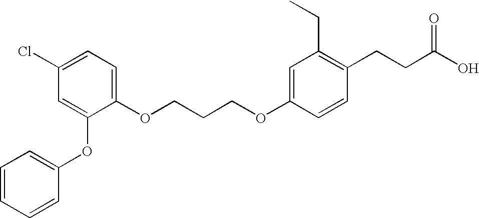 | 3-{4-[3-(4-Chloro-2-phenoxy-phenoxy)- propoxy]-2-ethyl-phenyl}-propionic acid |
| 202 | 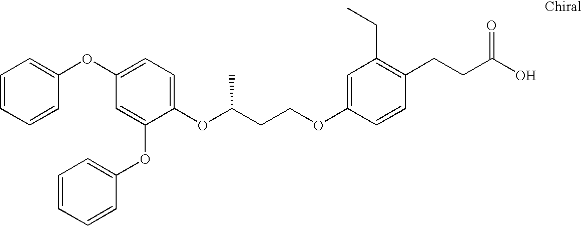 | (R)-3-{4-[3-(2,4-Diphenoxy-phenoxy)- butoxy]-2-ethyl-phenyl}-propionic acid |
| 203 | 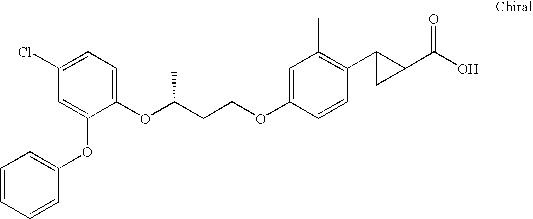 | 2-{4-[4-(4-Chloro-2-phenoxy-phenyl)- 3-methyl-butoxy]-2-methyl-phenyl}- cyclopropanecarboxylic acid |
| | Cis - Isomer 2 | |
| 204 | 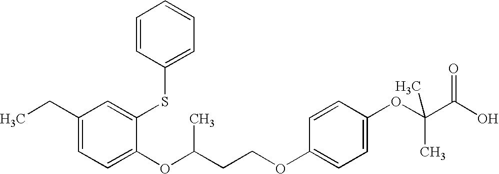 | (R, S)-2-{4-[3-(4-Ethyl-2-phenylsulfanyl- phenoxy)-butoxy]-phenoxy}-2-methyl- propionic acid |
| 205 | 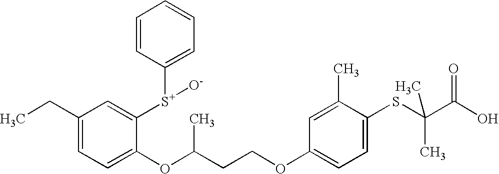 | 2-{4-[3-(R,S-2-Benzenesulfinyl-4-ethyl- phenoxy)-butoxy]-2-methyl-phenylsulfanyl}- 2-methyl-propionic acid (enamtiomer pair 1) |
| 206 | 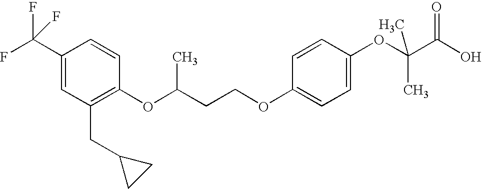 | (R, S)-2-{4-[3-(2-Cyclopropylmethyl-4- trifluoromethyl-phenoxy)-butoxy]-phenoxy}- 2-methyl-propionic acid |
| 207 | 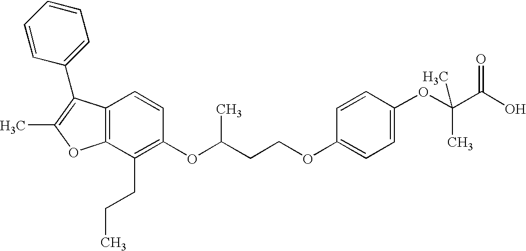 | (R, S)-2-Methyl-2-{4-[3-(2-methyl-3-phenyl- 7-propyl-benzofuran-6-yloxy)-butoxy]- phenoxy}-propionic acid |
| 208 | 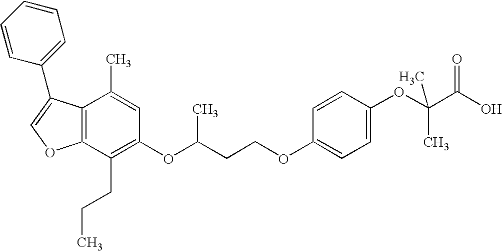 | (R, S)-2-Methyl-2-{4-[3-(4-methyl-3-phenyl- 7-propyl-benzofuran-6-yloxy)-butoxy]- phenoxy}-propionic acid |
| 209 | 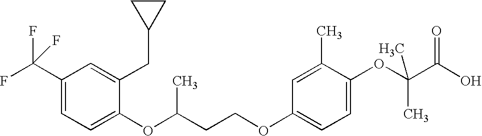 | (R, S)-2-{4-[3-(2-Cyclopropylmethyl-4- trifluoromethyl-phenoxy)-butoxy]-2- methyl-phenoxy}-2-methyl-propionic acid |
| 210 | 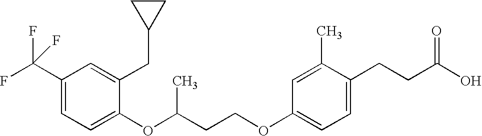 | (R, S)-3-{4-[3-(2-Cyclopropylmethyl-4- trifluoromethyl-phenoxy)-butoxy]-2- methyl-phenyl}-propionic acid |
| 211 | 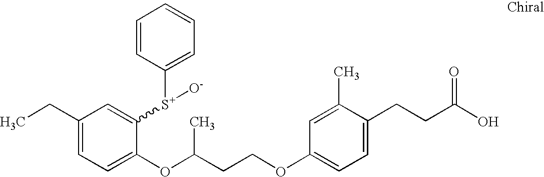 | 3-{R-4-[3-(R, S-2-Benzenesulfinyl-4-ethyl- phenoxy)-butoxy]-2-methyl-phenyl}- propionic acid |
| 212 | 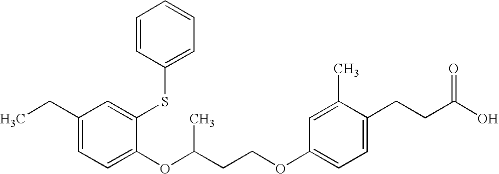 | 3-{4-[3-(4-Ethyl-2-phenylsulfanyl-phenoxy)- butoxy]-2-methyl-phenyl}-propionic acid isomer 2 |
| 213 | 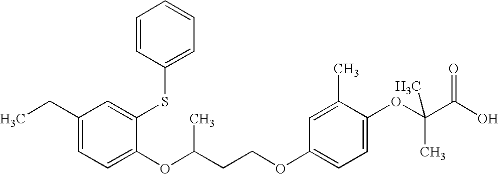 | (R, S)-2-{4-[3-(4-Ethyl-2-phenylsulfanyl- phenoxy)-butoxy]-phenoxy}-2-methyl- propionic acid |
| 214 | 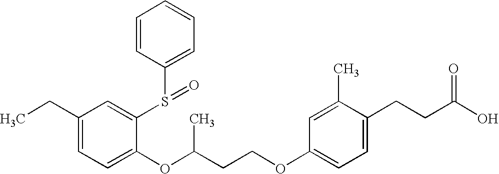 | (R, S)-3-{4-[3-(R,S-2-Benzenesulfinyl- 4-ethyl-phenoxy)-butoxy]-2-methyl-phenyl}- propionic acid |
| 215 | 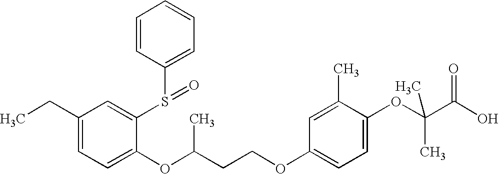 | (R, S)-2-{4-[3-(R,S-2-Benzenesulfinyl- 4-ethyl-phenoxy)-butoxy]-2-methyl- phenoxy}-2-methyl-propionic acid |
| 216 | 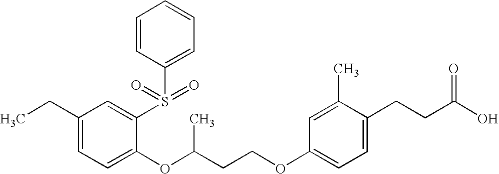 | (R, S)-3-{4-[3-(2-Benzenesulfonyl-4- ethyl-phenoxy)-butoxy]-2-methyl-phenyl}- propionic acid |
| 217 | 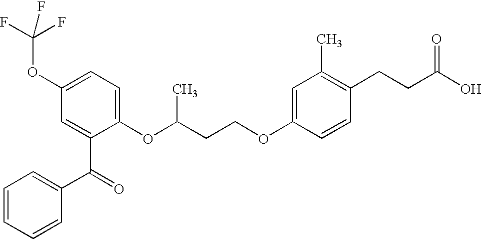 | 3-{4-[3-(2-Benzoyl-4-trifluoromethoxy- phenoxy)-butoxy]-2-methyl-phenyl}- propionic acid |

The compound as recited above, wherein the compound is 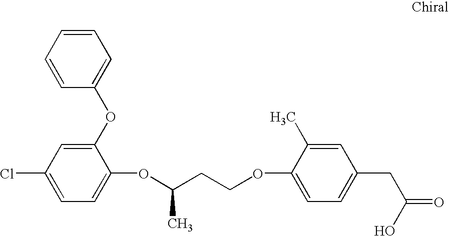
or a pharmaceutically acceptable salt, solvate or hydrate thereof.

Also encompassed by the present invention is a pharmaceutical composition comprising a pharmaceutically acceptable carrier and a compound of the present invention or a pharmaceutically acceptable salt, solvate or hydrate thereof.

Also encompassed by the present invention is a pharmaceutical composition comprising: (1) a compound of the present invention or a pharmaceutically acceptable salt, solvate, hydrate or stereoisomer thereof; (2) a second therapeutic agent selected from the group consisting of insulin sensitizers, sulfonylureas, biguanides, meglitinides, thiazolidinediones, α-glucosidase inhibitors, insulin secretogogues, insulin, antihyperlipidemic agents, plasma HDL-raising agents, HMG-CoA reductase inhibitors, statins, acryl CoA:cholestrol acyltransferase inhibitors, antiobesity compounds, antihypercholesterolemic agents, fibrates, vitamins and aspirin; and (3) optionally a pharmaceutically acceptable carrier.

Also encompassed by the present invention is a method of modulating a peroxisome proliferator activated receptor (PPAR) comprising the step of contacting the receptor with a compound of the present invention or a pharmaceutically acceptable salt, solvate or hydrate thereof.

The method recited above, wherein the PPAR is an alpha (α)-receptor.

The method recited above, wherein the PPAR is a gamma (γ)-receptor.

The method recited above, wherein the PPAR is a delta (δ)-receptor.

The method recited above, wherein the PPAR is a gamma/delta (γ/δ)-receptor.

The method recited above, wherein the PPAR is an alpha, gamma and delta (α/γ/δ)-receptor.

Also encompassed by the present invention is a method for treating and/or preventing a PPAR-γ mediated disease or condition in a mammal comprising the step of administering an effective amount of a compound of the present invention.

Also encompassed by the present invention is a method for treating and/or preventing a PPAR-δ mediated disease or condition in a mammal comprising the step of administering an effective amount of a compound of the present invention.

Also encompassed by the present invention is a method for treating and/or preventing a PPAR-γ/δ mediated disease or condition in a mammal comprising the step of administering an effective amount of a compound of the present invention.

Also encompassed by the present invention is a method for treating and/or preventing a PPAR-α/γ/δ mediated disease or condition in a mammal comprising the step of administering an effective amount of a compound of the present invention.

Also encompassed by the present invention is a method for lowering blood-glucose in a mammal comprising the step of administering an effective amount of a compound of the present invention.

Also encompassed by the present invention is a method of treating and/or preventing disease or condition in a mammal selected from the group consisting of hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component, comprising the step of administering an effective amount of a compound of the present invention.

Also encompassed by the present invention is a method of treating and/or preventing diabetes mellitus in a mammal comprising the step of administering to a mammal a therapeutically effective amount of a compound of the present invention.

Also encompassed by the present invention is a method of treating and/or preventing cardiovascular disease in a mammal comprising the step of administering to a mammal a therapeutically effective amount of a compound of the present invention, or a pharmaceutically acceptable salt, solvate, hydrate or stereoisomer thereof.

Also encompassed by the present invention is a method of treating and/or preventing syndrome X in a mammal comprising the step of administering to the mammal a therapeutically effective amount of a compound of the present invention, or a pharmaceutically acceptable salt, solvate, hydrate or stereoisomer thereof.

Also encompassed by the present invention is a method of treating and/or preventing disease or condition in a mammal selected from the group consisting of hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component, comprising the step of administering an effective amount of a compound of the present invention, and an effective amount of second therapeutic agent selected from the group consisting of insulin sensitizers, sulfonylureas, biguanides, meglitinides, thiazolidinediones, α-glucosidase inhibitors, insulin secretogogues, insulin, antihyperlipidemic agents, plasma HDL-raising agents, HMG-CoA reductase inhibitors, statins, acryl CoA:cholestrol acyltransferase inhibitors, antiobesity compounds, antihypercholesterolemic agents, fibrates, vitamins and aspirin.

Also encompassed by the present invention is use of a compound of the present invention and a pharmaceutically acceptable salt, solvate, hydrate or stereoisomer thereof, for the manufacture of a medicament for the treatment of a condition modulated by a PPAR.

Surprisingly, it is found that the compound having an alkyl branching (e.g., R¹ shown below) has unexpected activity (and/or selectivity) depending on the type of R¹ substituent (H vs. Me) and the conformation of R¹ substituent (R or S) as shown in Table 1 below.

TABLE 1

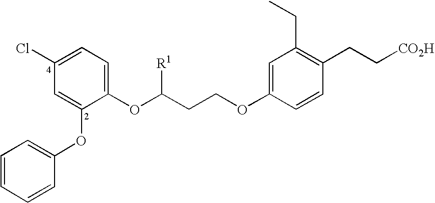

Effect of an alkyl branching (R¹ substituent)

| | EC₅₀α | EC₅₀γ | EC₅₀δ | | | |
|---|---|---|---|---|---|---|
| R¹ | (nM) | (nM) | (nM) | Eff % α | Eff % γ | Eff % δ |
| H | 2991 | 1291 | 18 | 35 | 89 | 143 |
| Me (R) | 2800 | 102 | 6 | 44 | 79 | 132 |
| Me (S) | NA | 2548 | 368 | 2 | 45 | 101 |

(NA = Not Active, no EC₅₀ is measured when Eff % is less than 20%)

As shown in Table 1, a notable improvement on gamma/delta dual agonist activity is achieved when the compound has an alkyl substituent (R¹=Me) adjacent to the 2,4-disubstituted phenoxy group. Additionally, improvement on gamma/delta dual agonist activity is more significant in R-enantiomer compare to its corresponding S-enantiomer.

Surprisingly, it is also noted that 2,4-disubstituted phenyl (Rc and Rd) of the compound shown below in Table 2 contributes significantly in achieving activity and/or selectivity of gamma/delta dual agonist.

TABLE 2

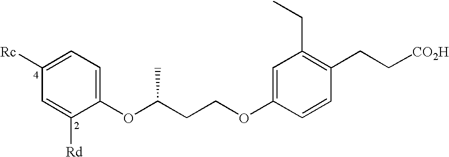

Effect of 2,4-disubstitution of phenyl ring

| | | EC₅₀α | EC₅₀γ | EC₅₀δ | | | |
|---|---|---|---|---|---|---|---|
| Rc | Rd | (nM) | (nM) | (nM) | Eff % α | Eff % γ | Eff % δ |
| H | H | NA | 2697 | 2952 | 4 | 24 | 43 |
| Cl | OPh | 2800 | 102 | 6 | 42 | 76 | 134 |

(NA Not Active, no EC₅₀ is measured when Eff % is less than 20%)

As shown in Table 2, a sharp loss of functional activity (>25 fold for EC₅₀γ and >400 fold for EC₅₀δ) is observed in an unsubstituted analog (Rc, Rd=H) compared to the corresponding 2,4-disubstituted analog (Rc=Cl, Rd=OPh).

It is further observed that when A attached to the position 2 of phenoxy group is a hydrogen-bond acceptor, there is an increase in activity (and/or selectivity) of gamma/delta dual agonist. Whereas when A is not a hydrogen-bond acceptor, a loss of activity on both receptors is observed as shown in the Table 3 below.

TABLE 3

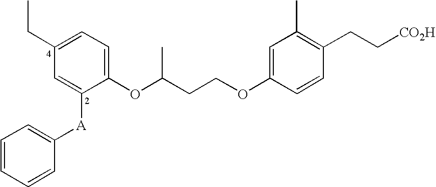

Effect of A attached to the position 2 of phenoxy group

| | EC₅₀α | EC₅₀γ | EC₅₀δ | | | |
|---|---|---|---|---|---|---|
| A | (nM) | (nM) | (nM) | Eff % α | Eff % γ | Eff % δ |
| CO | 1733 | 9 | 6 | 42 | 98 | 135 |
| C—NOH | 2872 | 7 | 6 | 38 | 83 | 131 |
| C—NOMe | 2662 | 644 | 35 | 30 | 139 | 130 |
| CHOH | 2872 | 7 | 6 | 38 | 83 | 131 |
| CH₂ | 2650 | 211 | 108 | 21 | 61 | 134 |
| C—CH₂ | NA | 272 | 67 | 13 | 75 | 139 |
| S | 2903 | 77 | 73 | 24 | 64 | 143 |
| SO | 1478 | 9 | 8 | 38 | 76 | 115 |
| O | 1414 | 8 | 5 | 37 | 80 | 121 |

Surprisingly, it is also found that a certain combination of X and Y are important in achieving the desired activity as shown in Table 4 below. For example, the combination of X/Y being O/CH₂ and O/S are more desirable for achieving a potent gamma/delta dual agonist activity.

TABLE 4

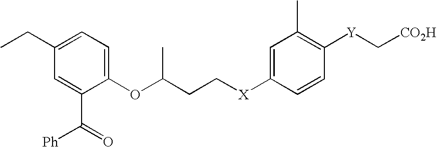

Effect of X/Y

| | | EC₅₀α | EC₅₀γ | EC₅₀δ | | | |
|---|---|---|---|---|---|---|---|
| X | Y | (nM) | (nM) | (nM) | Eff % α | Eff % γ | Eff % δ |
| O | CH₂ | 1503 | 9 | 7 | 40 | 80 | 120 |
| O | S | 2822 | 9 | 24 | 37 | 80 | 101 |
| O | O | 3035 | 89 | 38 | 37 | 76 | 102 |
| S | O | 2890 | 262 | 23 | 39 | 63 | 112 |
| S | CH₂ | 2838 | 172 | 39 | 25 | 83 | 121 |

The terms used to describe the present invention have the following meanings unless otherwise indicated.

The term “alkyl,” unless otherwise indicated, refers to those alkyl groups of a designated number of carbon atoms of either a straight or branched saturated configuration, including substituted alkyl. The term “alkyl” used herein also includes “alkylene group” of either straight or branched saturated configuration, including substituted alkylene. Examples of “alkyl” include, but are not limited to: methyl, ethyl, n-propyl, isopropyl, n-butyl, isobutyl, sec-butyl and tert-butyl, pentyl, hexyl, isopentyl and the like. Examples of “branched alkyl” (or “substituted alkyl”) include, but are not limited to —C(R¹)C(R^{9a})(R^{9b})CR²—; —C(R¹)C(R^{1a})(R^{b})CH₂CR²—; —C(R¹)CH₂C(R^{9a})(R^{9b})CH₂CR²—; —C(R¹)CH₂C(R^{9a})(R^{9b})(CH₂)₂CR²—; and the like where at least one of R^{9a} and R^{9b} is alkyl as defined above. Examples of “alkylene group” is —(CH₂)ₘ—, wherein m is a positive integer. Preferably, m is an integer from about 1 to about 6, more preferably from about 1 to about 3. A “branched (or substituted) alkylene group” is an alkylene group in which one or more methylene hydrogen atoms are replaced with a substituent, such as methyl, ethyl or the like. Alkyl as defined above may be optionally substituted with a designated number of substituents as set forth in the embodiment recited above.

The term “alkoxy” represents an alkyl group of indicated number of carbon atoms attached through an oxygen bridge, such as methoxy, ethoxy, propoxy, isopropoxy, butoxy, tert-butoxy, pentoxy, and the like. Alkoxy as defined above may be optionally substituted with a designated number of substituents as set forth in the embodiment recited above.

The term “cycloalkyl” refers to a saturated or partially saturated carbocycle containing one or more rings of from 3 to 12 carbon atoms, more typically 3 to 6 carbon atoms. Examples of cycloalkyl includes, but are not limited to cyclopropyl, cyclobutyl, cyclopentyl, cyclohexyl and cycloheptyl, and the like. Cycloalkyl as defined above may also includes a tricycle, such as adamantyl. Cycloalkyl as defined above may be optionally substituted with a designated number of substituents as set forth in the embodiment recited above.

The term “halo” refers to fluoro, chloro, bromo and iodo.

The term “haloalkyl” is a C₁-C₆ alkyl group, which is substituted with one or more halo atoms selected from F, Br, Cl and I. Examples of haloalkyl group are trifluoromethyl, CH₂CF₃ and the like.

The term “haloalkyloxy” represents a C₁-C₆ haloalkyl group attached through an oxygen bridge, such as OCF₃. The “haloalkyloxy” as defined above may be optionally substituted with a designated number of substituents as set forth in the embodiment recited above.

The term “aryl” includes carbocyclic aromatic ring systems (e.g. phenyl), fused polycyclic aromatic ring systems (e.g. naphthyl and anthracenyl) and aromatic ring systems fused to carbocyclic non-aromatic ring systems (e.g., 1,2,3,4-tetrahydronaphthyl). The “aryl” as defined above may be optionally substituted with a designated number of substituents as set forth in the embodiment recited above.

The term “aryloxy” represents an aryl group attached through an oxygen bridge, such as phenoxy (—O-phenyl). The “aryloxy” as defined above may be optionally substituted with a designated number of substituents as set forth in the embodiment recited above.

The term “heteroaryl” group, as used herein, is an aromatic ring system having at least one heteroatom such as nitrogen, sulfur or oxygen and includes monocyclic, bicyclic or tricyclic aromatic ring of 5- to 14-carbon atoms containing one or more heteroatoms selected from O, N, or S. The heteroaryl as defined above also includes heteroaryl fused with another heteroaryl, aryl fused with heteroaryl or aryl fused with heterocyclyl (e.g., benzo[1,4]dioxinyl) as defined herein. The “heteroaryl” may also be optionally substituted with a designated number of substituents as set forth in the embodiment recited above. Examples of heteroaryl are, but are not limited to: furanyl, thienyl (also referred to as “thiophenyl”), thiazolyl, imidazolyl, indolyl, isoindolyl, isooxazolyl, oxazoyl, pyrazolyl, pyrrolyl, pyrazinyl, pyridyl, pyrimidyl, pyrimidinyl and purinyl, cinnolinyl, benzofuranyl, benzothienyl (or benzothiophenyl), benzotriazolyl, benzoxazolyl, quinoline, isoxazolyl, isoquinoline 1,4 benzodioxan, or 2,3-dihydrobenzofuranyl and the like.

The term “bi-aryl” is defined as aryl substituted with another aryl or aryl substituted with heteroaryl as defined above. Examples of “biaryl” are, but are not limited to: bi-phenyl where phenyl is substituted with another phenyl; phenyl-pyridyl where phenyl is substituted with pyridyl; and phenyl-pyrimidinyl where phenyl is substituted with pyrimidinyl. Examples of “biaryl” also include “aryl-T-aryl” or “aryl-T-heteroaryl” where T is a bond, —(CH₂)_{q}O—, O(CH₂)_{q}—, C(O)(CH₂)_{q}—, —(CH₂)_{q}C(O)—, —(CH₂)_{q}S—, —S(CH₂)_{q}—, S[O]ₚ, —(C₁-C₃ alkyl)-, —(CH₂)_{q}C(═CH₂)—, —C(═CH₂)(CH₂)_{q}—, —(CH₂)_{q}C(═NOH)—, —C(═NOH)(CH₂)_{q}—, —(CH₂)_{q}C(═NOCH₃)—, —C(═NOCH₃)(CH₂)_{q}—, —CH(OH)(CH₂)_{q}— or —(CH₂)_{q}CH(OH)—; and q is 0, 1, 2 or 3.

The “bi-aryl” as defined above may be optionally substituted with a designated number of substituents as set forth in the embodiment recited above.

The term “bi-heteroaryl” is defined as heteroaryl substituted with another heteroaryl, or heteroaryl substituted with aryl or biaryl as defined above. Examples of “bi-heteroaryl” are, but are not limited to: thienyl-pyrazolyl, thienyl-thienyl, thienyl-pyridyl, thienyl-phenyl, thienyl-biphenyl and the like. Examples of “bi-heteroaryl” also include “heteroaryl-T-heteroaryl” or “heteroaryl-T-aryl” where T is a bond, —(CH₂)_{q}O—, —O(CH₂)_{q}—, —C(O)(CH₂)_{q}—, —(CH₂)_{q}C(O)—, —(CH₂)_{q}S—, —S(CH₂)_{q}—, S[O]ₚ, —(C₁-C₃ alkyl)-, —(CH₂)_{q}C(═CH₂)—, —C(═CH₂)(CH₂)_{q}—, —(CH₂)_{q}C(═NOH)—, —C(NOH)(CH₂)_{q}—, —(CH₂)_{q}C(═NOCH₃)—, —C(═NOCH₃)(CH₂)_{q}—, —CH(OH)(CH₂)_{q}— or —(CH₂)_{q}CH(OH)—; and q is 0, 1, 2 or 3. The “bi-heteroaryl” as defined above may be optionally substituted with a designated number of substituents as set forth in the embodiment recited above.

The term “heterocyclyl” refers to a non-aromatic ring which contains one or more heteroatoms selected from O, N or S, which includes a monocyclic, bicyclic or tricyclic ring of 5- to 14-carbon atoms containing one or more heteroatoms selected from O, N or S. The “heterocyclyl” as defined above may be optionally substituted with a designated number of substituents as set forth in the embodiment recited above. Examples of heterocyclyl include, but are not limited to, morpholine, piperidine, piperazine, pyrrolidine, and thiomorpholine.

The term “carbocyclyl” (also referred as “nonaromatic carbocyclic ring”) refers to a saturated or partially saturated nonaromatic carbocyclic ring. Examples of carbocyclyl are, but are not limited to, cyclopentyl, cyclohexyl, cyclopentenyl, cyclohexenyl and the like.

An “arylalkyl” as used herein is an aryl substituent that is linked to a compound by an alkyl group having from one to six carbon atoms. The “arylalkyl” as defined above may be optionally substituted with a designated number of substituents as set forth in the embodiment recited above.

The “aminoalkyl” as used herein contains both a basic amino group (NH₂) and an alkyl group as defined above.

The term R^{6A} (or acid bioisosteres) as used herein includes, but are not limited to, carboxamide, sulfonamide, acylsulfonamide, tetrazole or the following moiety.

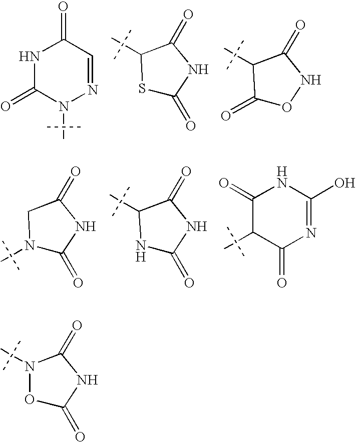

Carboxamide, sulfonamide, acylsulfonamide and tetrazole may be optionally substituted with one or more suitable substituents selected from haloalkyl, aryl, heteroaryl, and C₁-C₆ alkyl. The heteroalkyl, aryl, heteroaryl and alkyl may further optionally substituted with one or more substituents selected from the list provided for R¹⁵. The examples of R^{6A} (or acid bioisosteres) are, but not limited to, hydroxamic acid, acyl cyanamide, tetrazoles, sulfinylazole, sulfonylazole, 3-hydroxyisoxazole, hydroxythiadiazole, sulphonate and acylsulfonamide.

The term “active ingredient” means the compounds generically described by Formula I as well as the salts, solvates and prodrugs of such compounds.

The term “pharmaceutically acceptable” means that the carrier, diluents, excipients and salt must be compatible with the other ingredients of the composition, and not deleterious to the recipient thereof. Pharmaceutical compositions of the present invention are prepared by procedures known in the art using well-known and readily available ingredients.

“Preventing” refers to reducing the likelihood that the recipient will incur or develop any of the pathological conditions described herein.

“Treating” refers to mediating a disease or condition, and preventing or mitigating its further progression or ameliorating the symptoms associated with the disease or condition.

“Pharmaceutically-effective amount” means that amount of a compound of the present invention, or of its salt, solvate, hydrate or prodrug thereof that will elicit the biological or medical response of a tissue, system or mammal. Such an amount can be administered prophylactically to a patient thought to be susceptible to development of a disease or condition. Such amount when administered prophylactically to a patient can also be effective to prevent or lessen the severity of the mediated condition. Such an amount is intended to include an amount, which is sufficient to modulate a PPAR receptor such as a PPARα, PPARγ, PPARδ or PPARγ/δ receptor to mediate a disease or condition. Conditions mediated by PPAR receptors include, for example, diabetes mellitus, cardiovascular disease, Syndrome X, obesity and gastrointestinal disease. Additional conditions associated with the modulation of a PPAR receptor include inflammation related conditions, which include, for example, IBD (inflammatory bowel disease), rheumatoid arthritis, psoriasis, Alzheimer's disease, Chrohn's disease and ischemia reprofusion injury (stroke and miocardial infarction).

A “mammal” is an individual animal that is a member of the taxonomic class Mammalia. The class Mammalia includes humans, monkeys, chimpanzees, gorillas, cattle, swine, horses, sheep, dogs, cats, mice, rats and the like.

Administration to a human is most preferred. A human to whom the compounds and compositions of the present invention are administered has a disease or condition in which control blood glucose levels are not adequately controlled without medical intervention, but wherein there is endogenous insulin present in the human's blood. Non-insulin dependent diabetes mellitus (NIDDM) is a chronic disease or condition characterized by the presence of insulin in the blood, even at levels above normal, but resistance or lack of sensitivity to insulin action at the tissues.

Those skilled in the art will recognize that sterocenters exist in compound of the present invention. Accordingly, the present invention includes all possible stereoisomers and geometric isomers of the presently claimed compounds including racemic compounds and the optically active isomers.

The compounds of the present invention contain one or more chiral centers and exist in different optically active forms. When compounds of the present invention contain one chiral center, the compounds exist in two enantiomeric forms and the present invention includes both enantiomers and mixtures of enantiomers, such as racemic mixtures. Resolution of the final product, an intermediate or a starting material may be effected by any suitable method known in the art, for example by formation of, diastereoisomeric salts which may be separated by crystallization; formation of diastereoisomeric derivatives or complexes which may be separated by crystallization and gas-liquid or liquid chromatography; selective reaction of one enantiomer with an enantiomer-specific reagent such as enzymatic esterification; and gas-liquid or liquid chromatography in a chiral environment such as on a chiral support, for example silica with a bound chiral ligand or in the presence of a chiral solvent. See also Sterochemistry of Carbon Compounds by E. L. Eliel (Mcgraw Hill, 1962) and Tables of Resolving Agents by S. H. Wilen. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the present invention has more than one chiral substituents, it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of the present invention may exist in different stable conformational forms, which may be separable. Torsional asymmetry due to restricted rotation about an asymmetric single bond, for example because of steric hindrance or ring strain, may permit separation of different conformers. The present invention includes each conformational isomer of compounds of formula I and mixtures thereof.

Certain compound of the present invention may exist in zwitterionic form, and the present invention includes each zwitterionic form of compounds of formula I and mixtures thereof.

Certain compounds of the present invention and their salts may exist in more than one crystal form. Polymorphs of compounds of formula I form part of the present invention and may be prepared by crystallization of a compound of formula I under different conditions, such as using different solvents or different solvent mixtures for recrystallization; crystallization at different temperatures; and various modes of cooling ranging from very fast to very slow cooling during crystallization. Polymorphs may also be obtained by heating or melting a compound of formula I followed by gradual or fast cooling. The presence of polymorphs may be determined by solid probe NMR spectroscopy, IR spectroscopy, differential scanning calorimetry, powder X-ray diffraction or other available techniques.

Certain compounds of the present invention and their salts may exist in more than one crystal form, which includes each crystal form and mixtures thereof.

Certain compounds of the present invention and their salts may also exist in the form of solvates, for example hydrates, and thus the present invention includes each solvate and mixtures thereof.

“Pharmaceutically-acceptable salt” refers to salts of the compounds of formula I, which are substantially non-toxic to mammals. Typical pharmaceutically acceptable salts include those salts prepared by reaction of the compounds of the present invention with a mineral, organic acid: an organic base or inorganic base. Such salts are known as base addition salts, respectively. It should be recognized that the particular counterion forming a part of any salt of the present invention is not of a critical nature so long as the salt as a whole is pharmaceutically acceptable and the counterion does not contribute undesired qualities to the salt as a whole.

By virtue of its acidic moiety, a compound of the present invention forms salts with pharmaceutically acceptable bases. Some examples of base addition salts include metal salts such as aluminum; alkali metal salts such as lithium, sodium or potassium; and alkaline earth metal salts such as calcium, magnesium, ammonium, or substituted ammonium salts. Examples of substituted ammonium salts include, for instance, those with lower alkylamines such as trimethylamine and triethylamine; hydroxyalkylamines such as 2-hydroxyethylamine, bis-(2-hydroxyethyl)-amine or tri-(2-hydroxyethyl)-amine; cycloalkylamines such as bicyclohexylamine or dibenzylpiperidine, N-benzyl-β-phenethylamine, dehydroabietylamine, N,N′-bisdehydro-abietylamine, glucamine, N-piperazine methylglucamine; bases of the pyridine type such as pyridine, collidine, quinine or quinoline; and salts of basic amino acids such as lysine and arginine.

Examples of inorganic bases include, without limitation, sodium hydroxide, potassium hydroxide, potassium carbonate, sodium carbonate, sodium bicarbonate, potassium bicarbonate, calcium hydroxide, calcium carbonate, and the like.

Compounds of the present invention, which are substituted with a basic group, may exist as salts with pharmaceutically acceptable acids. The present invention includes such salts. Examples of such salts include hydrochlorides, hydrobromides, sulfates, methanesulfonates, nitrates, maleates, acetates, citrates, fumarates, tartrates [e.g. (+)-tartrates, (−)-tartrates or mixtures thereof including racemic mixtures], succinates, benzoates and salts with amino acids such as glutamic acid. These salts may be prepared by methods known to those skilled in the art.

Certain compounds of the present invention and their salts may also exist in the form of solvates, for example hydrates, and thus the present invention includes each solvate and mixtures thereof.

The compounds of present invention, which bind to and activate the PPARs, lower one or more of glucose, insulin, triglycerides, fatty acids and/or cholesterol, and are therefore useful for the treatment and/or prevention of hyperglycemia, dyslipidemia and in particular Type II diabetes as well as other diseases including syndrome X, Type I diabetes, hypertriglyceridemia, insulin resistance, diabetic dyslipidemia, hyperlipidemia, hypercholesteremia, heart failure, coagaulopathy, hypertension, and cardiovascular diseases, especially arteriosclerosis. In addition, these compounds are indicated to be useful for the regulation of appetite and food intake in subjects suffering from disorders such as obesity, anorexia bulimia and anorexia nervosa.

The compounds and compositions of the present invention are also useful to treat acute or transient disorders in insulin sensitivity, which sometimes occurs following a surgery, trauma, myocardial infarction and the like. The compounds and compositions of the present invention are also useful for lowering serum triglyceride levels. Elevated triglyceride level, whether caused by genetic predisposition or by a high fat diet, is a risk factor for the development of heart disease, stroke, and circulatory system disorders and diseases. The physician of ordinary skill will know how to identify humans who can benefit from administration of the compounds and compositions of the present invention.

The present invention further provides a method for the treatment and/or prophylaxis of hyperglycemia in a human or non-human mammal which comprises administering an effective, non-toxic amount of a compound of formula I, or a tautomeric form thereof and/or a pharmaceutically acceptable salt thereof and/or a pharmaceutically acceptable solvate thereof to a hyperglycemic human or non-human mammal in need thereof.

The compounds of the present invention are useful as therapeutic substances in preventing or treating Syndrome X, diabetes mellitus and related endocrine and cardiovascular disorders and diseases in human or non-human animals.

The present invention also relates to the use of a compound of formula I as described above for the manufacture of a medicament for treating a PPARγ or PPARδ mediated condition, separately or in combination.

A therapeutically effective amount of a compound of the present invention can be used for the preparation of a medicament useful for treating Syndrome X, diabetes, treating obesity, lowering tryglyceride levels, raising the plasma level of high density lipoprotein, and for treating, preventing or reducing the risk of developing arteriosclerosis, and for preventing or reducing the risk of having a first or subsequent atherosclerotic disease event in mammals, particularly in humans. In general, a therapeutically effective amount of a compound of formula I of the present invention could reduce serum glucose level (or HbAlc) of a patient by about 0.7% or more; and reduce serum triglyceride level by about 15% or more and increases serum HDL level in a patient by 20% or more.

Additionally, an effective amount of a compound of the present invention and a therapeutically effective amount of one or more active agents selected from antihyperlipidemic agent, plasma HDL-raising agents, antihypercholesterolemic agents, fibrates, vitamins, aspirin, insulin secretogogues, insulin and the like can be used together for the preparation of a medicament useful for the above described treatments.

Advantageously, compositions containing the compound of the present invention or their salts may be provided in dosage unit form, preferably each dosage unit containing from about 1 to about 500 mg. It is understood that the amount of the compounds or compounds of the present invention that will be administered is determined by a physician considering of all the relevant circumstances.

Syndrome X includes pre-diabetic insulin resistance syndrome and the resulting complications thereof, insulin resistance, non-insulin dependent diabetes, dyslipidemia, hyperglycemia obesity, coagulopathy, hypertension and other complications associated with diabetes. The methods and treatments mentioned herein include the above and encompass the treatment and/or prophylaxis of any one of or any combination of the following: pre-diabetic insulin resistance syndrome, the resulting complications thereof, insulin resistance, Type II or non-insulin dependent diabetes, dyslipidemia, hyperglycemia, obesity and the complications associated with diabetes including cardiovascular disease, especially arteriosclerosis.

The compositions are formulated and administered in the same general manner as detailed herein. The compounds of the present invention may be used effectively alone or in combination with one or more additional active agents depending on the desired target therapy. Combination therapy includes administration of a single pharmaceutical dosage composition, which contains a compound of the present invention and one or more additional active agents, as well as administration of a compound of the present invention and each active agent in its own separate pharmaceutical dosage. For example, a compound of the present invention or thereof and an insulin secretogogue such as biguanides, meglitinides, thiazolidinediones, sulfonylureas, insulin or α-glucosidose inhibitors can be administered to the patient together in a single oral dosage composition such as a tablet or capsule, or each agent administered in separate oral dosages. Where separate dosages are used, a compound of the present invention and one or more additional active agents can be administered at essentially the same time, i.e., concurrently or at separately staggered times, i.e., sequentially; combination therapy is understood to include all these regimens.

An example of combination treatment or prevention of arteriosclerosis may involve administration of a compound of the present invention or salts thereof in combination with one or more of second active therapeutic agents: antihyperlipidemic agents; plasma HDL-raising agents; antihypercholesterolemic agents, fibrates, vitamins, aspirin and the like. As noted above, the compounds of the present invention can be administered in combination with more than one additional active agent.

Another example of combination therapy can be seen in treating diabetes and related disorders wherein the compounds of the present invention or salts thereof can be effectively used in combination with second active therapeutic, such as sulfonylureas, biguanides, meglitinides, thiazolidinediones, α-glucosidase inhibitors, other insulin secretogogues, insulin as well as the active agents discussed above for treating arteriosclerosis.

The examples of second therapeutic agents are insulin sensitizers, PPARγ agonists, glitazones, troglitazone, pioglitazone, englitazone, MCC-555, BRL 49653, biguanides, metformin, phenformin, insulin, insulin minetics, sufonylureas, tolbutamide, glipizide, alpha-glucosidase inhibitors, acarbose, cholesterol lowering agent, HMO-CoA reductase inhibitors, lovastatin, simvastatin, pravastatin, fluvastatin, atrovastatin, rivastatin, other statins, sequestrates, cholestyramine, colestipol, dialkylaminoalkyl derivatives of a cross-linked dextran, nicotinyl alcohol, nicotinic acid: a nicotinic acid salt, PPARα agonists, fenofibric acid derivatives, gemfibrozil, clofibrate, fenofibrate, benzafibrate, inhibitors of cholesterol absorption, beta-sitosterol, acryl CoA:cholesterol acyltransferase inhibitors, melinamide, probucol, PPARδ agonists, antiobesity compounds, fenfluramine, dexfenfluramine, phentiramine, sulbitramine, orlistat, neuropeptide Y5 inhibitors, β₃ adrenergic receptor agonists, and ileal bile acid transporter inhibitors.

The compounds of the present invention and the pharmaceutically acceptable salts, solvates and hydrates thereof have valuable pharmacological properties and can be used in pharmaceutical compositions containing a therapeutically effective amount of a compound of the present invention, or pharmaceutically acceptable salts, esters or prodrugs thereof, in combination with one or more pharmaceutically acceptable excipients. Excipients are inert substances such as, without limitation carriers, diluents, fillers, flavoring agents, sweeteners, lubricants, solubilizers, suspending agents, wetting agents, binders, disintegrating agents, encapsulating material and other conventional adjuvants. Proper excipient is dependent upon the route of administration chosen. Pharmaceutical compositions typically contain from about 1 to about 99 weight percent of the active ingredient, which is a compound of the present invention.

Preferably, the pharmaceutical formulation is in unit dosage form. A “unit dosage form” is a physically discrete unit containing a unit dose suitable for administration in human subjects or other mammals. For example, a unit dosage form can be a capsule or tablet, or a number of capsules or tablets. A “unit dose” is a predetermined quantity of the active compound of the present invention, calculated to produce the desired therapeutic effect, in association with one or more pharmaceutically acceptable excipients. The quantity of active ingredient in a unit dose may be varied or adjusted from about 0.1 to about 1000 milligrams or more according to the particular treatment involved.

The dosage regimen utilizing the compounds of the present invention is selected by one of ordinary skill in the medical or veterinary arts considering various factors, such as without limitation, the species, age, weight, sex, medical condition of the recipient, the severity of the condition to be treated, the route of administration, the level of metabolic and excretory function of the recipient, the dosage form employed, the particular compound and salt thereof employed, and the like.

Preferably, the compounds of the present invention are administered in a single daily dose, or the total daily dose may be administered in divided doses of two, three or more times per day. Where delivery is via transdermal forms, administration is continuous.

Suitable routes of administration of pharmaceutical compositions of the present invention include, for example, oral, eye drop, rectal, transmucosal, topical or intestinal administration; parenteral delivery (bolus or infusion), including intramuscular, subcutaneous, intramedullary injections, as well as intrathecal, direct intraven-tricular, intravenous, intraperitoneal, intranasal, or intraocular injections. The compounds of the present invention can also be administered in a targeted drug delivery system, such as in a liposome coated with endothelial cell-specific antibody.

For oral administration, the compounds of the present invention can be formulated readily by combining the active compounds with pharmaceutically acceptable carriers well known in the art. Such carriers enable the compounds of the present invention to be Formulated as tablets, pills, powders, sachets, granules, dragees, capsules, liquids, elixirs, tinctures, gels, emulsions, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores.

For oral administration in the form of a tablet or capsule, the active ingredient may be combined with an oral, non-toxic, pharmaceutically-acceptable carrier, such as, without limitation, lactose, starch, sucrose, glucose, methyl cellulose, calcium carbonate, calcium phosphate, calcium sulfate, sodium carbonate, mannitol, sorbitol, and the like; together with, optionally, disintegrating agents, such as, without limitation, cross-linked polyvinyl pyrrolidone, maize, starch, methyl cellulose, agar, bentonite, xanthan gum, alginic acid: or a salt thereof such as sodium alginate, and the like; and, optionally, binding agents, for example, without limitation, gelatin, acacia, natural sugars, beta-lactose, corn sweeteners, natural and synthetic gums, acacia, tragacanth, sodium alginate, carboxymethyl-cellulose, polyethylene glycol, waxes, and the like; and, optionally, lubricating agents, for example, without limitation, magnesium stearate, sodium stearate, stearic acid: sodium oleate, sodium benzoate, sodium acetate, sodium chloride, talc, and the like. When a dosage unit form is a capsule, it may contain, in addition to materials of the above type, a liquid carrier such as a fatty oil.

Solid forms include powders, tablets and capsules. A solid carrier can be one or more substances, which may also act as flavoring agents, lubricants, solubilisers, suspending agents, binders, tablet disintegrating agents and encapsulating material.

In powders, the carrier is a finely divided solid, which is in admixture with the finely divided active ingredient. In tablets, the active ingredient is mixed with a carrier having the necessary binding properties in suitable proportions and compacted in the shape and size desired.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Sterile liquids include suspensions, emulsions, syrups, and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

All formulations for oral administration should be in dosages suitable for such administration. Particularly suitable compositions for oral administration are unit dosage forms such as tablets and capsules.

For parental administration, the compounds of the present invention or salts thereof can be combined with sterile aqueous or organic media to form injectable solutions or suspensions. Formulations for injection may be presented in unit dosage form, such as in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that each syringability exists. It must be stable under the conditions of manufacture and storage and must be preserved against any contamination. The carrier can be solvent or dispersion medium containing, for example, water, preferably in physiologically compatible buffers such as Hanks' solution, Ringer's solution, or physiological saline buffer, ethanol, polyol (e.g. glycerol, propylene glycol and liquid polyethylene glycol), propylene glycol and liquid polyethylene glycol), suitable mixtures thereof, and vegetable oils. Under ordinary conditions of storage and use, these preparations contain a preservative to prevent the growth of microorganisms.

The injectable solutions prepared in this manner can then be administered intravenously, intraperitoneally, subcutaneously, or intramuscularly, with intramuscular administration being preferred in humans.

For transmucosal administration, penetrants appropriate to the barrier to be permeated are used in the formulation. Such penetrants are generally known in the art. The active compounds can also be administered intranasally as, for example, liquid drops or spray.

For buccal administration, the compositions may take the form of tablets or lozenges Formulated in a conventional manner.

For administration by inhalation, the compounds for use according to the present invention are conveniently delivered in the form of a dry powder inhaler, or an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of gelatin for use in an inhaler or insufflator may be formulated containing a powder mix of the compound and a suitable powder base such as lactose or starch.

Pharmaceutical compositions of the present invention can be manufactured in a manner that is itself known, e.g., by means of conventional mixing, dissolving, granulating, dragee-making, levigating, emulsifying, encapsulating, entrapping or lyophilizing processes.

In making the compositions of the present invention, the active ingredient will usually be admixed with a carrier, or diluted by a carrier, or enclosed within a carrier, which may be in the form of a capsule, sachet, paper or other container. When the carrier serves as a diluent, it may be a solid, lyophilized solid or paste, semi-solid, or liquid material which acts as a vehicle, or can be in the form of tablets, pills, powders, lozenges, elixirs, suspensions, emulsions, solutions, syrups, aerosols (as a solid or in a liquid medium), or ointment, containing for example up to 10% by weight of the active compound. The compounds of the present invention are preferably formulated prior to administration.

Binding and Cotransfection Studies

The in vitro potency of compounds in modulating PPARγ, PPARα and PPARδ receptors are determined by the procedures detailed below. DNA-dependent binding (ABCD binding) is carried out using Scintillation Proximity Assay (SPA) technology with PPAR receptors. Tritium-labeled PPARα and PPARγ agonists are used as radioligands for generating displacement curves and IC₅₀ values with compounds of the present invention. Cotransfection assays are carried out in CV-1 cells. The reporter plasmid contains an acylCoA oxidase (AOX) PPRE and TK promoter upstream of the luciferase reporter cDNA. Appropriate PPARs and RXRα are constitutively expressed using plasmids containing the CMV promoter. Since for PPARα and PPARβ, interference by endogenous PPARγ in CV-1 cells is an issue, in order to eliminate such interference, a GAL4 chimeric system is used in which the DNA binding domain of the transfected PPAR is replaced by that of GAL4, and the GAL4 response element is utilized in place of the AOX PPRE. Receptor activation by compounds of the present invention is determined relative to PPARα agonist and PPARγ agonist reference molecules to obtain percent efficacies. EC50 values are determined by computer fit to a concentration-response curve. A typical range for concentration determination is from 1 nM to 10 μM. For binding or cotransfection studies with receptors other than PPARs, similar assays are carried out using appropriate ligands, receptors, reporter constructs and etc. for that particular receptor. In some cases, a single high concentration of agonist (10 μM) was used.

These studies are carried out to evaluate the ability of compounds of the present invention to bind to and/or activate various nuclear transcription factors, particularly huPPARα (“hu” indicates “human”), huPPARγ and huPPARδ. These studies provide in-vitro data concerning efficacy and selectivity of compounds of the present invention. Furthermore, binding and cotransfection data for compounds of the present invention are compared with corresponding data for reference compounds that act on either huPPARα or huPPARγ. The typical range of concentration for binding is from 1 nM to 10 μM. The concentration of test compound required to effect 50% maximal activation of PPARα (IC₅₀α) and PPARγ (IC₅₀γ) is determined. The compounds of the present invention are, in general, found to have IC₅₀ in the range of about 1 nM to about 5 μM for PPAR gamma and/or delta.

Evaluation of Triglyceride and Cholesterol Level in HuapoAI Transgenic Mice

Five to six week old male mice, transgenic for human apoAI [C57Bl/6-tgn(apoa1)1rub, Jackson Laboratory, Bar Harbor, Me.] are housed five per cage (10″×20″×8″ with aspen chip bedding) with food (Purina 5001) and water available at all times. After an acclimation period of 2 weeks, animals are individually identified by ear notches, weighed and assigned to groups based on body weight. Beginning the following morning, mice are dosed daily by oral gavage for 7 days using a 20 gauge, 1½″ curved disposable feeding needle. Treatments are test compounds (30 mg/kg), a positive control (fenofibrate, 100 mg/kg) or vehicle [1% carboxymethylcellulose (w/v)/0.25% Tween80 (w/v); 0.2 ml/mouse]. Prior to termination on day 7, mice are weighed and dosed. Three hours after dosing, animals are anesthetized by inhalation of isoflurane (2-4%) and blood obtained via cardiac puncture (0.7-1.0 ml). Whole blood is transferred to serum separator tubes (Vacutainer SST), chilled on ice and permitted to clot. Serum is obtained after centrifugation at 4° C. and frozen until analysis for triglycerides, total cholesterol, compound levels and serum lipoprotein profile by fast protein liquid chromatography (FPLC) coupled to an inline detection system. After sacrifice by cervical dislocation, the liver, heart and epididymal fat pads are excised and weighed.

The animals dosed with vehicle have average triglycerides values of about 60 to 80 mg/dl, which are reduced by the positive control fenofibrate (33-58 mg/dl with a mean reduction of 37%). The animals dosed with vehicle have average total serum cholesterol values of about 140 to 180 mg/dl, which are increased by fenofibrate (about 190 to 280 mg/dl with a mean elevation of 41%). When subject to FPLC analysis, pooled sera from vehicle-treated hu apoAI transgenic mice have a high-density lipoprotein cholesterol (HDLc) peak area, which ranges from 47 v-sec to 62 v-sec. Fenofibrate increases the amount of HDLc (68-96 v-sec with a mean percent increase of 48%). Test compounds evaluated in terms of percent increase in the area under the curve. Representative compounds of the present invention are tested using the above methods or substantially similar methods.

Evaluation of Glucose Levels in db/db Mice

Five week old male diabetic (db/db) mice [C57BlKs/j-m+/+Lepr(db), Jackson Laboratory, Bar Harbor, Me.] or lean littermates (db+) are housed 6 per cage (10″×20″×8″ with aspen chip bedding) with food (Purina 5015) and water available at all times. After an acclimation period of 2 weeks, animals are individually identified by ear notches, weighed and bled via the tail vein for determination of initial glucose levels. Blood is collected (100 μl) from unfasted animals by wrapping each mouse in a towel, cutting the tip of the tail with a scalpel, and milking blood from the tail into a heparinized capillary tube balanced on the edge of the bench. Sample is discharged into a heparinized microtainer with gel separator (VWR) and retained on ice. Plasma is obtained after centrifugation at 4° C. and glucose is measured immediately. Remaining plasma is frozen until the completion of the experiment, and glucose and triglycerides are assayed in all samples. Animals are grouped based on initial glucose levels and body weights. Beginning the following morning, mice are dosed daily by oral gavage for 7 days using a 20 gauge, 1½″ curved disposable feeding needle. Treatments are test compounds (30 mg/kg), a positive control agent (30 mg/kg) or vehicle [1% carboxymethylcellulose (w/v)/0.25% Tween80 (w/v); 0.3 ml/mouse]. On day 7, mice are weighed and bled (tail vein) for about 3 hours after dosing. Twenty-four hours after the 7^{th} dose (i.e., day 8), animals are bled again (tail vein). Samples obtained from conscious animals on days 0, 7 and 8 are assayed for glucose. After 24 hour bleed, animals are weighed and dosed for the final time. Three hours after dosing on day 8, animals are anesthetized by inhalation of isoflurane, and blood obtained is via cardiac puncture (0.5-0.7 ml). Whole blood is transferred to serum separator tubes, chilled on ice and permitted to clot. Serum is obtained after centrifugation at 4° C. and frozen until analysis for compound levels. After sacrifice by cervical dislocation, the liver, heart and epididymal fat pads are excised and weighed.

The animals dosed with vehicle have average triglycerides values of about 170 to 230 mg/dl, which are reduced by the positive PPARγ control (about 70 to 120 mg/dl with a mean reduction of 50%). Male db/db mice are hyperglycemic (average glucose of about 680 to 730 mg/dl on the 7^{th} day of treatment), while lean animals have average glucose levels between about 190 and 230 mg/dl. Treatment with the positive control agent reduces glucose significantly (about 350 to 550 mg/dl with a mean decrease towards normalization of 56%).

Glucose is measured colorimetrically by using commercially purchased reagents (Sigma #315-500). According to the manufacturers, the procedures are modified from published work (McGowan et al. Clin Chem, 20:470-5 (1974) and Keston, A. Specific colorimetric enzymatic analytical reagents for glucose. Abstract of papers 129th Meeting ACS, 31C (1956).); and depend on the release of a mole of hydrogen peroxide for each mole of analyte coupled with a color reaction first described by Trinder (Trinder, P. Ann Clin Biochem, 6:24 (1969)). The absorbance of the dye produced is linearly related to the analyte in the sample. The assays are further modified for use in a 96 well format. Standards (Sigma #339-11, Sigma #16-11, and Sigma #CC0534 for glucose, triglycerides and total cholesterol, respectively), quality control plasma (Sigma # A2034), and samples (2 or 5 μl/well) are measured in duplicate using 200 μl of reagent. An additional aliquot of sample, pipetted to a third well and diluted in 200 μl water, provided a blank for each specimen. Plates are incubated at room temperature (18, 15, and 10 minutes for glucose, triglycerides and total cholesterol, respectively) on a plate shaker and absorbance read at 500 nm (glucose and total cholesterol) or 540 nm (triglycerides) on a plate reader. Sample absorbance is compared to a standard curve (100-800, 10-500, and 100-400 mg/dl for glucose, triglycerides and total cholesterol, respectively). Values for the quality control sample are consistently within the expected range and the coefficient of variation for samples is below 10%. All samples from an experiment are assayed at the same time to minimize inter-assay variability.

Serum lipoproteins are separated and cholesterol is quantitated with an in-line detection system. Sample is applied to a Superose® 6 HR 10/30-size exclusion column (Amersham Pharmacia Biotech) and eluted with phosphate buffered saline-EDTA at 0.5 ml/min. Cholesterol reagent (Roche Diagnostics Chol/HP 704036) at 0.16 ml/min is mixed with the column effluent through a T-connection, and the mixture is passed through a 15 m×0.5 mm id knitted tubing reactor immersed in a 37° C. water bath. The colored product produced in the presence of cholesterol is monitored in the flow stream at 505 nm, and the analog voltage from the monitor is converted to a digital signal for collection and analysis. The change in voltage corresponding to change in cholesterol concentration is plotted against time, and the area under the curve corresponding to the elution of VLDL, LDL and HDL is calculated (Perkin Elmer Turbochrome software).

The compounds of the present invention can be prepared according to the procedures of the following schemes and examples, which may further illustrate details for the preparation of the compounds of the present invention. The compounds illustrated in the schemes and examples are, however, not to be construed as forming the only genus that is considered as the present invention.

General Reaction Scheme

The compounds of the present invention, in general, may be prepared according to the Reaction Schemes described below.

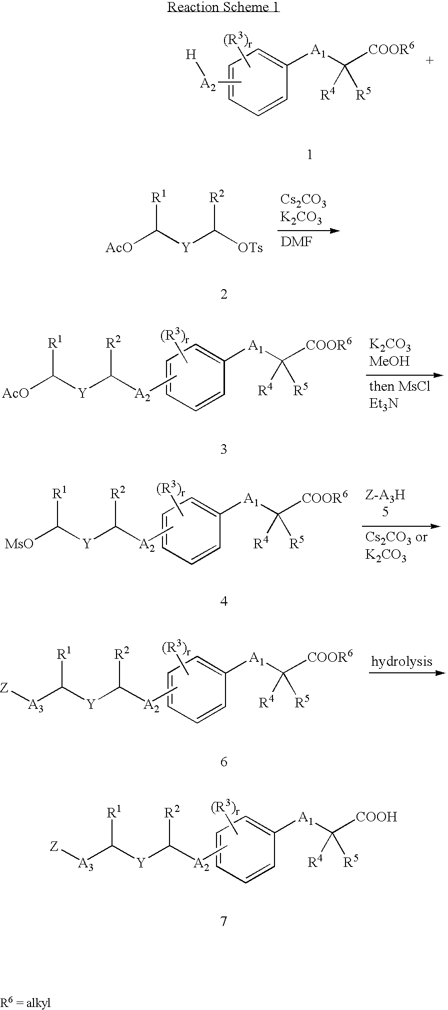

As shown in Reaction Scheme 1, treatment of tosylate 2 with a headpiece compound 1 under the basic condition provides intermediate 3. Acetyl group is removed under K₂CO₃/MeOH condition followed by mesylation of the free alcohol to afford compound 4. Final tailpiece (Z-A₃H) is installed by treatment of compound 4 with compound 5 to give ester 6, which is then undergoes a hydrolysis to provide final acid 7.

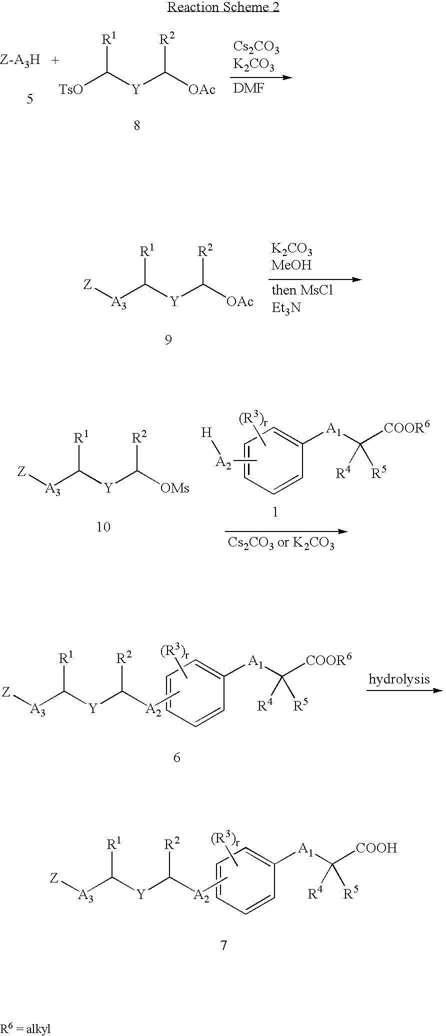

As shown in Reaction Scheme 2, treatment of tosylate 8 with a tailpiece compound 5 under the basic condition provides intermediate 9. Acetyl group is removed under K₂CO₃/MeOH condition followed by mesylation of the free alcohol to afford compound 10. Final headpiece (compound 1) is installed by treatment of compound 10 with 1 to give ester 6, which is then undergoes a hydrolysis to provide final acid 7.

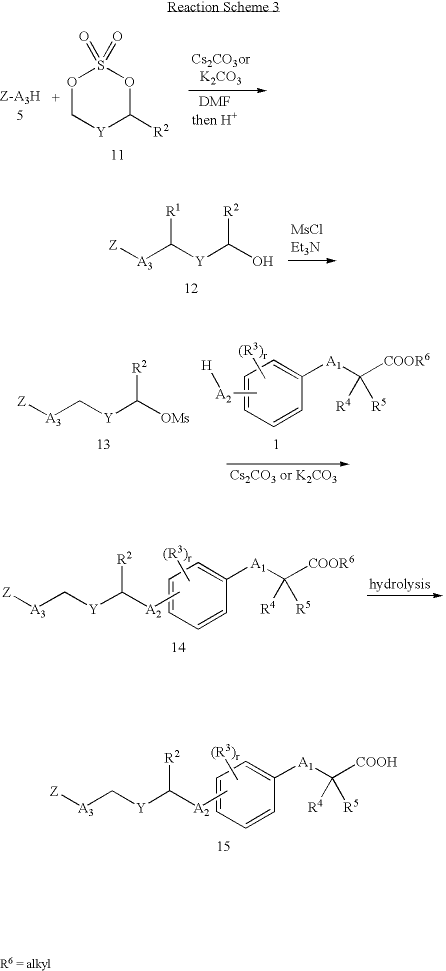

As shown in Reaction Scheme 3, treatment of cyclic sulfate 11 with compound 5 under the basic condition provides alcohol 12. Mesylation of the free alcohol affords compound 13. Final headpiece (compound 1) is installed by treatment of compound 13 with 1 to give ester 14, which is then undergoes, a hydrolysis to provide final acid 15.

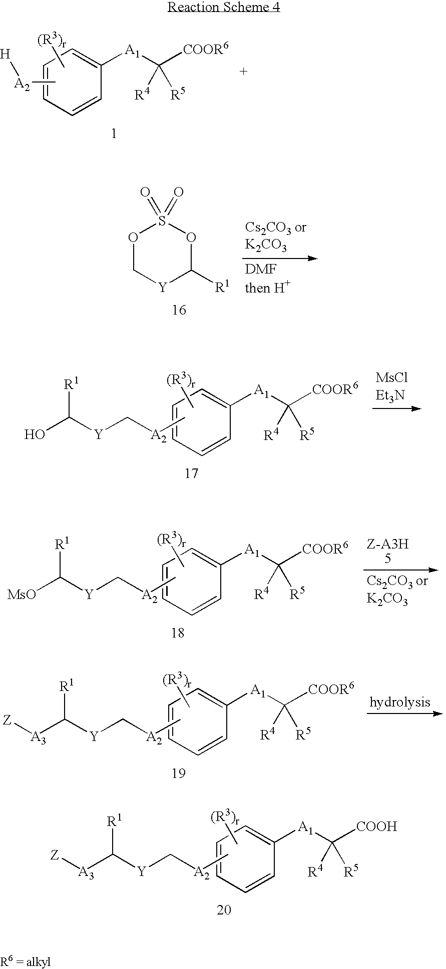

As shown in Reaction Scheme 4, treatment of cyclic sulfate 16 with headpiece 1 under the basic condition provides alcohol 17. Mesylation of the free alcohol affords compound 18. Final tailpiece (Z-A₃H) is installed by treatment of compound 18 with compound 5 to give ester 19, which is then undergoes a hydrolysis to provide final acid 20.

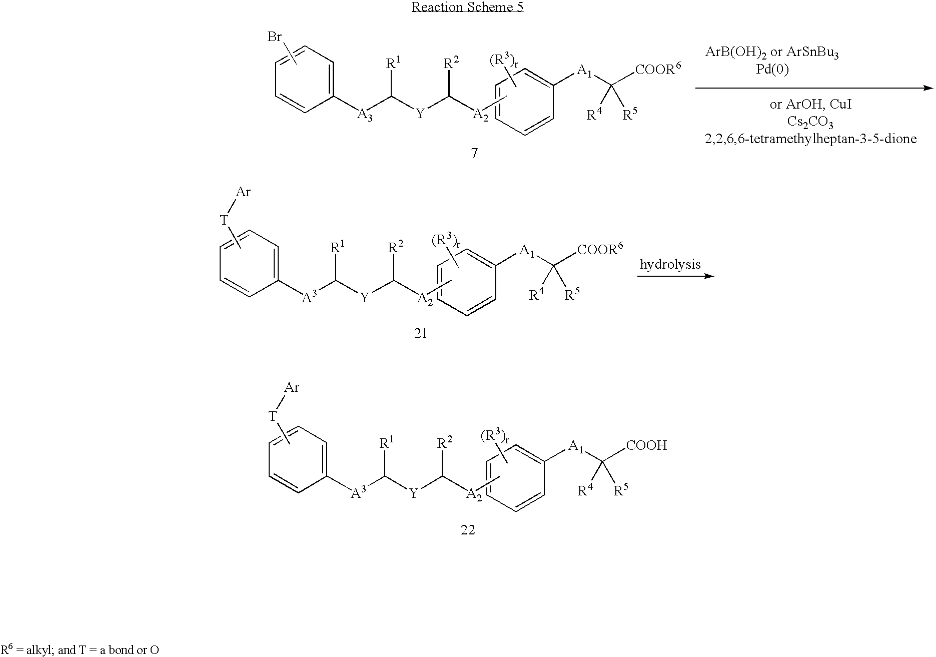

Compound 7 is prepared according to the procedure described in Reaction Schemes 1-4. As shown in Reaction Scheme 5, the moiety of -T-Ar such as aryl or aryloxy groups in 21, which is prepared from the parent bromide compound 7, is installed by using standard Suzuki, Stille or Ullmann reaction conditions. A hydrolysis of ester compound 21 provides final acid 22.

In the Schemes, Procedures and Examples below, various reagent symbols and abbreviations have the following meanings.

| | |
|---|---|
| ACN | Acetonitrile |
| BINAP | 2,2′-Bis(diphenylphosphino)-1,1′-binaphthyl |
| Boc | t-butoxycarbonyl |
| CBZ | benzyloxycarbonyl |
| DCM | dichloromethane |
| DEAD | diethyl azodicarboxylate |
| DIAD | diisopropyl azodicarboxylate |
| DIPEA | diisopropylethylamine |
| DMAP | 4-dimethylamino pyridine |
| DMF | N,N-dimethylformamide |
| DMSO | dimethylsulfoxide |
| eq (equiv) | equivalent(s) |
| ESI-MS | electron spray ion-mass spectroscopy |
| Et | ethyl |
| EtOAc | ethyl acetate |
| h | hours |
| HOAc | acetic acid |
| HPLC | high performance liquid chromatography |
| HRMS | high resolution mass |
| LRMS | low resolution mass |
| LAH | lithium aluminum hydride |
| Me | methyl |
| Ms | methanesulfonyl |
| NBS | N-bromosuccinimide |
| Pd₂(dba)₃ | tris(dibenzylideneacetone)dipalladium(0) |
| Ph | phenyl |
| Pr | propyl |
| rt (r.t.) | room temperature |
| TBAI | tetrabutylammonium iodide |
| TBS | tertbutyldimethylsilyl |
| TFA | trifluoroacetic acid |
| TEA | triethylamine |
| THF | tetrahydrofuran |
| TLC | thin-layer chromatography |

### EXAMPLE 1

### 2-{4-[3-(2-benzoyl-4-ethyl-phenoxy)-butoxy]-2-methyl-phenoxy}-2-methyl-propionic acid

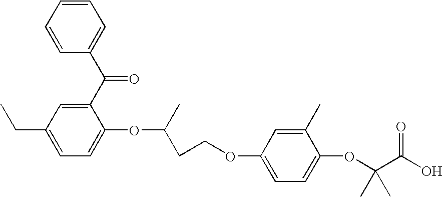

### Step A

### Acetic acid 3-(toluene-4-sulfonyloxy)-butyl ester

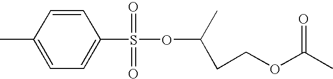

TEA (0.88 mL, 3.79 mmol), p-toluenesulfonyl chloride (0.72 g, 3.79 mmol) and 4-dimethylaminopyridine (0.09 g, 0.79 mmol) are added to acetic acid 3-hydroxy-butyl ester (0.41 g, 3.16 mmol) in dichloromethane (DCM) (4 mL) at 0° C. under N₂, and the mixture is stirred for an hour at 0° C. The mixture is warmed gradually to ambient temperature. After 24 h, the mixture is treated with water and extracted with EtOAc. The organic layers are combined and washed with saturated aqueous sodium chloride, and then dried over magnesium sulfate and concentrated under reduced pressure. Purification by flash chromatography, silica, eluting with hexanes: EtOAc (75:25) afforded the title compound (0.71 g, 2.48 mmol, 78%) as a white solid: ES⁺ (m/e) 304.19 (M+NH₄)⁺; ¹H NMR (400 MHz, CDCl₃) 7.79 (d, 2H, J=8 Hz), 7.33 (d, 2H, J=8 Hz), 4.65-4.80 (m, 1H), 3.85-4.20 (m, 2H), 2.44 (s, 3H), 1.96 (s, 3H), 1.80-1.95 (m, 2H), 1.34 (d, 3H, J=6.4 Hz); R_{f}=0.40 hexanes: EtOAc (70:30).

### Step B

### 5-Ethyl-2-hydroxy-phenyl-methanone

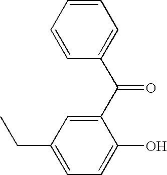

Aluminum chloride (0.58 g, 4.4 mmol) is added in portions to p-ethylanisole (0.50 g, 3.7 mmol) in DCM (3.4 mL) at 0° C. under N₂, and the mixture is stirred for about 10 minutes, and then benzoyl chloride (0.43 mL, 3.9 mmol) is added dropwise. The mixture is stirred at 0° C. for 4 h and poured in ice. The mixture is warmed to ambient temperature and extracted with EtOAc. Organic layers are combined and washed with aqueous sodium chloride, dried over magnesium sulfate, filtered and concentrated to obtain yellow oil. Crude mixture is dissolved in toluene (4.3 mL) and aluminum chloride (0.49 g, 3.7 mmol) is added in portions at ambient temperature, and stirred under N₂. The mixture is warmed at 80° C. for 3 h and additional 16 h at 55° C. The mixture is cooled to ambient temperature and poured in ice. The mixture is extracted with EtOAc. Organic phases are combined and washed with aqueous sodium chloride, and then dried over magnesium sulfate, filtered and concentrated under reduced pressure. Purification by flash chromatography, silica, eluting with hexanes: EtOAc (98:2) provides the title compound as a yellow oil that crystallizes with the time: ES⁺ (m/e) 227.10 (M+H)⁺, R_{f}=0.65 hexanes: EtOAc (90:10).

### Step C

### Acetic acid 3-(2-benzoyl-4-ethyl-phenoxy)-butyl ester

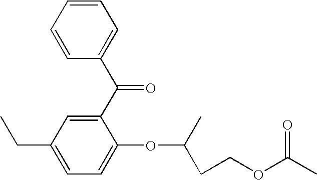

Cesium carbonate (0.72 g, 2.22 mmol) is added to (5-ethyl-2-hydroxy-phenyl)-phenyl-methanone (0.50 g, 2.22 mmol) and acetic acid 3-(toluene-4-sulfonyloxy)-butyl ester (0.60 g, 2.09 mmol) in DMF (DMF) (7.5 mL) at ambient temperature under N₂, and the mixture is stirred at 55° C. for 16 h. The mixture is cooled to ambient temperature, diluted with water and extracted with EtOAc. The organic phase is combined, dried over magnesium sulfate, filtered and concentrated under reduced pressure. Purification by flash chromatography, silica, eluting with hexanes: EtOAc (89:11) provides the title compound as a colorless oil (0.58 g, 1.71 mmol), 82%): ES⁺ (m/e) 341.24 (M+H)⁺, 363.24 (M+Na)⁺; R_{f}=0.40 hexanes: EtOAc (80:20); ¹H NMR (400 MHz, CDCl₃) 7.76 (m, 2H), 7.50-7.54 (m, 1H), 7.39-7.43 (m, 2H), 7.23-7.27 (m, 2H), 6.85 (d, 1H, J=8.4 Hz) 2.62 (q, 2H, J=7.6 Hz), 1.99 (s, 3H), 1.62-1.67 (m, 2H), 1.22 (t, 3H, J=7.6 Hz), 1.11 (d, 3H, J=6 Hz).

### Step D

### [5-Ethyl-2-(3-hydroxy-1-methyl-propoxy-)phenyl]-phenyl-methanone

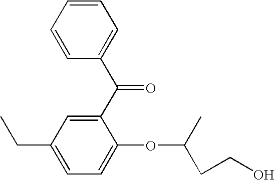

Potassium carbonate (0.15 g, 1.09 mmol) is added to acetic acid 3-(2-benzoyl-4-ethyl-phenoxy)-butyl ester (0.58 g, 1.71 mmol) in methanol (4.5 mL) at room temperature, and the mixture is stirred. After 5 h, the mixture is diluted with water and extracted with EtOAc. Organic layers are combined and washed with saturated aqueous sodium chloride, and then dried over magnesium sulfate, filtered and concentrated under reduced pressure the title compound as a colorless oil (0.50 g, 1.67 mmol, 98%): ES⁺ (m/e), 299.22 (M+H)⁺, 321.24 (M+Na)⁺; R_{f}=0.20 hexanes: EtOAc (80:20); ¹H NMR (400 MHz, CDCl₃) 7.78-7.81 (m, 2H), 7.53-7.58 (m, 1H), 7.40-7.44 (m, 2H), 7.26 (dd, 1H, J₁=2.4 Hz, J₂=8.4 Hz), 7.20 (d, 1H, J=2.8 Hz), 6.93 (d, 1H, J=8.4 Hz), 4.50-4.65 (m, 1H), 3.50-3.70 (m, 2H), 2.61 (q, 2H, J=7.2 Hz), 1.78 (bs,1H), 1.62-1.75 (m, 2H), 1.21 (t, 3H, J=7.2 Hz), 1.17 (d, 3H, J=6.4 Hz).

### Step E

### 2-{4-[3-(2-Benzoyl-4-ethyl-phenoxy)-butoxy]-2-methyl-phenoxy}-2-methyl-propionic acid

Triphenylphosphine (46 mg, 0.17 mmol) is added to [5-ethyl-2-(3-hydroxy-1-methyl-propoxy-)phenyl]-phenyl-methanone (34 mg, 0.12 mmol) and 2-(4-hydroxy-2-methyl-phenoxy)-2-methyl-propionic acid ethyl ester (42 mg, 0.17 mmol) in toluene (1.3 mL) under N₂ at ambient temperature. Diethylazodicarboxilate (34 μL, 0.17 μmol) is added dropwise, and the mixture is stirred for 16 h. The mixture is concentrated under reduced pressure. Purification by flash chromatography, silica, hexanes: EtOAc (90:10) provides 2-{4-[3-(2-benzoyl-4-ethyl-phenoxy)-butoxy]-2-methyl-phenoxy}-2-methyl-propionic acid ethyl ester (43 mg, 0.08 mmol, 71%): ES⁺ (m/e) 519.3 (M+H)⁺; R_{f}=0.59 hexanes: EtOAc (80:20).

Aqueous solution of sodium hydroxide (5M, 0.13 mL, 0.67 mmol) is added to the above propionic acid ethyl ester (35 mg, 0.07 mmol) in ethanol, and the mixture is stirred for 5 h at ambient temperature. The mixture is acidified to pH=2 with a 1 M HCl, and extracted with EtOAc. Organic layers are combined and washed with saturated aqueous sodium chloride, and then dried over magnesium sulfate, filtered and concentrated under reduced pressure to afford the title compound (33 mg, 0.07 mmol, 100%): ES⁺ (m/e) 491.3 (M+H)⁺; ¹H NMR (400 MHz, CDCl₃) 7.76 (d, 2H, J=7,6 Hz), 7.47-7.50 (m, 1H), 7.34-7.37 (m, 2H), 7.20-7.25 (m, 2H), 6.88 (d, J=8.4 Hz), 6.70-6.80 (m, 1H), 6.48-6.56 (m, 2H), 4.53-4.60 (m, 1H), 3.66 (m, 2H), 2.61 (q, 2H, J=7.6 Hz), 2.18 (bs, 3H), 1.61 (bs, 6H), 1.27-1.50 (m, 2H), 1.22 (t, 3H, J=7.6 Hz), 1.15 (t, 3H, J=5.6 Hz).

### EXAMPLE 2

### 3-{4-[3-(2-benzoyl-4-ethyl-phenoxy)-butoxy]2-methyl-phenyl}propionic acid

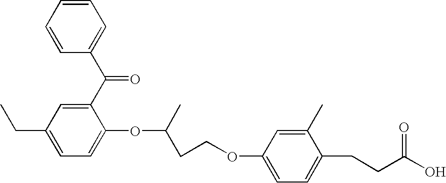

The compound of 3-{4-[3-(2-benzoyl-4-ethyl-phenoxy)-butoxy]-2-methyl-phenyl}-propionic acid methyl ester (21 mg, 0.05 mmol, 76%) is prepared by following the procedure described in Example 1, Step E by using triphenylphosphine (23 mg, 0.09 mmol), [5-ethyl-2-(3-hydroxy-1-methyl-propoxy-)phenyl]-phenyl-methanone (17 mg, 0.06 mmol) (Example 1, Step D), 3-(4-hydroxy-2-methyl-phenyl-propionic acid methyl ester (17 mg, 0.09 mmol) and diethylazodicarboxilate (17 μL, 0.09 mmol). ES⁺ (m/e) 475.29 (M+H)⁺, 497.29 (M+Na)⁺; R_{f}=0.42 hexanes: EtOAc (80:20).

Work up of the above propionic acid methyl ester (21 mg, 0.04 mmol) in methanol (0.5 mL) as described in Example 1, Step E provides the title compound (20 mg, 0.04 mmol, 100%): ES⁺ (m/e) 461.27 (M+H)⁺, 483.26 (M+Na)⁺; ¹H NMR (400 MHz, CDCl₃) 7.76 (d, 2H, J=7.6 Hz), 7.48-7.52 (m, 1H), 7.35-7.39 (m, 2H), 7.21-7.25 (m, 2H), 7.00 (d, J=8.4 Hz), 6.88 (d, 1H, J=8.4 Hz), 6.58 (d, 1H, J=6.58 Hz), 6.52 (dd, 1H, J=2 Hz, J₂=8.4 Hz), 4.50-4.60 (m, 1H), 3.68 (t, 2H, J=6 Hz), 2.87 (t, 2H, J=8.4 Hz), 2.57-2.64 (m, 4H), 2.27 (s, 3H), 1.75-1.81 (m, 2H), 1.21 (t, 3H, J=7.6 Hz), 1.15 (d, 3H, J=6.4 Hz).

### EXAMPLE 3

### 2-{3-[3-(2-benzoyl-4-ethyl-phenoxy)-butoxy]-phenoxy}-2-methyl-propionic acid

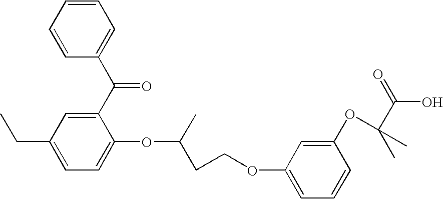

The compound of 2-{3-[3-(2-benzoyl-4-ethyl-phenoxy)-butoxy]-phenoxy}-2-methyl-propionic acid ethyl ester (17 mg, 0.03 mmol, 56%) is prepared by following the procedure described in Example 1, Step E by using triphenylphosphine (24 mg, 0.09 mmol), [5-ethyl-2-(3-hydroxy-1-methyl-propoxy-)phenyl]-phenyl-methanone (18 mg, 0.06 mmol) (Step D of Example 1), 2-(3-hydroxy-phenoxy)-2-methyl-propionic acid ethyl ester (20 mg, 0.09 mmol) in toluene (0.5 mL) and diethylazodicarboxilate (18 μL, 0.09 mmol). ES⁺ (m/e) 505.30 (M+H)⁺, R_{f}=0.49 hexanes: EtOAc (80:20).

Work-up of the above propionic acid ethyl ester (17 mg, 0.04 mmol) in ethanol (0.5 mL) as described in Example 1, Step E provides the title compound as a colorless oil (16 mg, 0.04 mmol, 100%): ES⁺ (m/e) 477.29 (M+H)⁺, 499.26 (M+Na)⁺; ¹H NMR (400 MHz, CDCl₃) 7.78 (d, 2H, J=6.8 Hz), 7.50-7.54 (m, 1H), 7.37-7.41 (m, 2H), 7.19-7.25 (m, 2H), 7.10-7.14 (m, 1H), 6.88 (d, J=8.4 Hz), 6.49-6.51 (m, 1H), 6.41-6.42 (m, 1H), 4.50-4.60 (m, 1H), 3.72 (t, 2H, J=5.6 Hz), 2.61 (q, 2H, J=8 Hz), 1.72-1.90 (m, 2H), 1.59 (s, 3H), 1.59 (s, 3H), 1.22 (t, 3H, J=7.6 Hz), 1.17 (d, 3H, J=6 Hz).

### EXAMPLE 4

### 3-{4-[3-(2-Benzoyl-4-ethyl-phenoxy)-butoxy]-phenyl}-2-methoxy-propionic acid

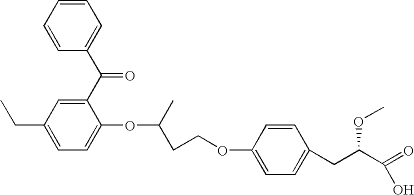

The compound of 3-{4-[3-(2-benzoyl-4-ethyl-phenoxy)-butoxy]-phenyl}-2-methoxy-propionic acid ethyl ester (24 mg, 0.05 mmol, 46%) is prepared by following the procedure described in Example 1, Step E by using triphenylphosphine (40 mg, 0.15 mmol), [5-ethyl-2-(3-hydroxy-1-methyl-propoxy-)phenyl]-phenyl-methanone (30 mg, 0.10 mmol) (Step D of Example 1), 3-{4-hydroxy-phenyl)-2-methoxy-propionic acid ethyl ester (34 mg, 0.15 mmol) in toluene (1.7 mL) and diethylazodicarboxilate (30 μL, 0.15 mmol). ES⁺ (m/e) 505.30 (M+H)⁺, R_{f}=0.40 hexanes: EtOAc (80:20).

Work-up of the above propionic acid ethyl ester (24 mg, 0.05 mmol) in ethanol (0.5 mL) as described in Example 1, Step E provides the title compound as a colorless oil (22 mg, 0.05 mmol, 100%): ES⁺ (m/e) 477.3 (M+H)⁺, 499.3 (M+Na)⁺; ¹H NMR (400 MHz, CDCl₃) 7.76-7.78 (m, 2H), 7.49-7.52 (m, 1H), 7.35-7.39 (m, 2H), 7.21-7.26 (m, 2H), 7.11 (d, 1H, J=8.4 Hz), 6.89 (d, 1H, J=8.4 Hz), 6.67-6.69 (m, 2H), 4.52-4.60 (m, 1H), 3.98 (dd, 1H, J=4 Hz, J₂=7.2 Hz), 3.65-3.73 (m, 2H), 3.40 (s, 3H), 2.93-3.11 (m, 2H), 2.61 (q, 2H, J=7.6 Hz), 1.74-1.82 (m, 2H), 1.22 (t, 3H, J=7.6 Hz), 1.16 (d, 3H, J=6.4 Hz).

### EXAMPLE 5

### 3-{3-[3-(2-Benzoyl-4-ethyl-phenoxy)-butoxyl]-phenyl}-2-m-ethoxy-propionic acid

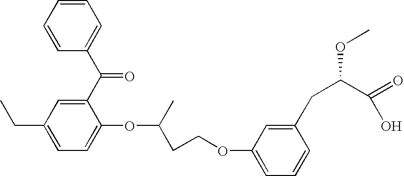

The compound of 3-{3-[3-(2-benzoyl-4-ethyl-phenoxy)-butoxyl]-phenyl}-2-m-ethoxy-propionic acid ethyl ester (12 mg, 0.03 mmol, 36%) is prepared by following the procedure described in Example 1, Step E by using rriphenylphosphine (26 mg, 0.10 mmol), [5-ethyl-2-(3-hydroxy-1-methyl-propoxy-)phenyl]-phenyl-methanone (20 mg, 0.07 mmol), 3-(3-hydroxy-phenyl)-2-methoxy-propionic acid ethyl ester (22 mg, 0.10 mmol) in toluene (0.7 mL) and diethylazodicarboxilate (19 μL, 0.10 mmol). ES⁺ (m/e) 505.3 (M+H)⁺, R_{f}=0.42 hexanes: EtOAc (80:20).

Work-up of the above propionic acid ethyl ester (12 mg, 0.03 mmol) in ethanol (0.4 mL) as described in Example 1, Step E provides the title compound as a colorless oil (11 mg, 0.03 mmol, 100%): ES⁺ (m/e) 477.3 (M+H)⁺, 499.3 (M+Na)⁺; ¹H NMR (400 MHz, CDCl₃) 7.77-7.79 (m, 2H), 7.50-7.54 (m, 1H), 7.37-7.41 (m, 2H), 7.14-7.26 (m, 3H), 6.89 (d, 1H, J=8.4 Hz), 6.80 (d, 1H, J=7.6 Hz), 6.63-6.73 (m, 2H), 4.54-4.60 (m, 1H), 3.99-4.03 (m, 1H), 3.73-3.76 (m, 2H), 3.39 (s, 3H), 2.9-3.12 (m, 2H), 2.61 (q, 2H, J=7.6 Hz), 1.74-1.88 (m, 2H), 1.22 (t, 3H, J=7.6 Hz), 1.17 (d, 3H, J=6.4 Hz).

### EXAMPLE 6

### {4-[3-(2-Benzoyl-4-ethyl-phenoxy)-butoxy]-2-methyl-phenoxy}-acetic acid

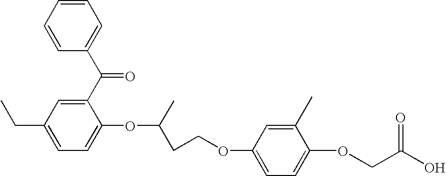

The compound of 3-{4-[3-(2-benzoyl-4-ethyl-phenoxy)-butoxy]-2-methyl-phenoxy}-acetic acid methyl ester (41 mg, 0.09 mmol, 86%) is prepared by following the procedure described in Step E of Example 1 by using triphenylphosphine (39 mg, 0.15 mmol), [5-ethyl-2-(3-hydroxy-1-methyl-propoxy-)phenyl]-phenyl-methanone (30 mg, 0.10 mmol), (4-hydroxy-2-methyl-phenoxy)-acetic acid methyl ester (29 mg, 0.15 mmol) in toluene (1.2 mL) and diethylazodicarboxilate (30 μL, 0.15 mmol). ES⁺ (m/e) 477.27 (M+H)⁺, 499.26 (M+Na)⁺; R_{f}=0.35 hexanes: EtOAc (80:20).

Work-up of the above acetic acid methyl ester (40 mg, 0.08 mmol) in methanol (1.0 mL) as described in Example 1, Step E provides the title compound as a colorless oil (38 mg, 0.08 mmol, 100%): ES⁺ (m/e) 463.26 (M+H)⁺; ¹H NMR (400 MHz, CDCl₃) 7.76-7.78 (m, 2H), 7.48-7.52 (m, 2H), 7.34-7.39 (m, 2H), 7.21-7.25 (m, 2H), 6.89 (d, 1H, J=8.4 Hz), 6.61-6.66 (m, 2H), 6.51 (dd, 1H, J₁=2.8 Hz, J₂=8.4 Hz), 4.61 (s, 2H), 4.54-4.60 (m, 1H), 3.65-3.68 (m, 2H), 2.61 (q, 2H, J=8 Hz), 2.25 (s, 3H), 1.73-1.82 (m, 2H), 1.22 (t, 3H, J=8 Hz), 1.16 (d, 3H, J=6 Hz).

### EXAMPLE 7

### {4-[3-(2-Benzoyl-4-ethyl-phenoxy)-butoxy]-phenoxy}acetic acid

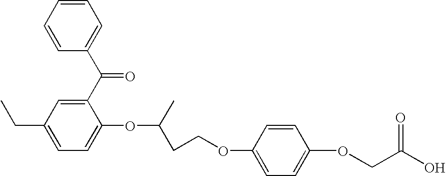

The compound of {4-[3-(2-benzoyl-4-ethyl-phenoxy)-butoxy]-phenoxy}acetic acid ethyl ester (29 mg, 0.06 mmol, 61%) is prepared by following the procedure described in Step E of Example 1 by using triphenylphosphine (39 mg, 0.15 mmol), [5-ethyl-2-(3-hydroxy-1-methyl-propoxy-)phenyl]-phenyl-methanone (30 mg, 0.10 mmol), (4-hydroxy-phenoxy)-acetic acid ethyl ester (29 mg, 0.15 mmol) in toluene (1.2 mL) and diethylazodicarboxilate (30 μL, 0.15 mmol). ES⁺ (m/e) 477.3 (M+H)⁺, R_{f}=0.39 hexanes: EtOAc (80:20).

Work up of the above acetic acid ethyl ester (29 mg, 0.06 mmol) in ethanol (1.0 mL) as described in Example 1, Step E provides the title compound as a colorless oil (27 mg, 0.06 mmol, 100%): ES⁺ (m/e) 449.28 (+H)⁺; ¹H NMR (400 MHz, CDCl₃) 7.76-7.78 (m, 2H), 7.48-7.52 (m, 1H), 7.36-7.39 (m, 2H), 7.21-7.25 (m, 2H), 6.89 (d, 1H, J=8.4 Hz), 6.82-6.84 (m, 2H), 6.68-6.71 (m, 2H), 4.62 (s, 2H), 4.54-4.58 (m, 1H), 3.67-3.70 (m, 2H), 2.61 (q, 2H, J=7.6 Hz), 1.75-1.84 (m, 2H), 1.22 (t, 3H, J=7.6 Hz), 1.16 (d, 3H, J=6.4 Hz).

### EXAMPLE 8

### {4-[3-(2-Benzoyl-4-ethyl-phenoxy)-butoxy]-2-methyl-phenyl-sulfanyl}-acetic acid

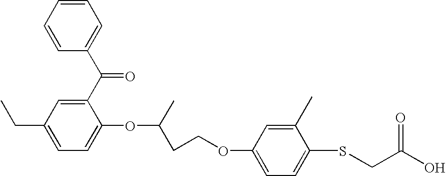

The compound of {4-[3-(2-benzoyl-4-ethyl-phenoxy)-butoxy]-2-methyl-phenylsulfanyl}-acetic acid ethyl ester (37 mg, 0.07 mmol, 72%) is prepared by following the procedure described in Step E of Example 1 by using rriphenylphosphine (39 mg, 0.15 mmol), [5-ethyl-2-(3-hydroxy-1-methyl-propoxy-)phenyl]-phenyl-methanone (30 mg, 0.10 mmol), (4-hydroxy-2-methyl-nphenylsulfanyl)-acetic acid ethyl ester (34 mg, 0.15 mmol) in toluene (1.2 mL) and diethylazodicarboxilate (30 μL, 0.15 mmol). ES⁺ (m/e) 507.26 (M+H)⁺, R_{f}=0.43 hexanes: EtOAc (80:20).

Work up of the above acetic acid ethyl ester (37 mg, 0.08 mmol) in ethanol (1.0 mL) as described in Example 1, Step E provides the title compound as a colorless oil (34 mg, 0.06 mmol, 100%): ES⁺ (m/e) 479.23 (M+H)⁺; ¹H NMR (400 MHz, CDCl₃) 7.76-7.78 (m, 2H), 7.45-7.52 (m, 1H), 7.35-7.39 (m, 2H), 7.205-7.25 (m, 2H), 6.86 (d, 1H, J=8.4 Hz), 6.64 (d, 1H, J=2.4 Hz), 6.54 (dd, 1H, J₁=2.4 Hz, J₂=8.8 Hz), 4.52-4.59 (m, 1H), 3.67-3.71 (m, 1H), 3.48 (s, 2H), 2.61 (q, 2H, J=7.6 Hz), 2.42 (s, 3H), 1.74-1.85 (m, 1H), 1.22 (t, 3H, J=7.6 Hz), 1.16 (d, J=6 Hz).

### EXAMPLE 9

### {4-[3-(2-Benzoyl-4-ethyl-phenoxy)-butyl]-2-methyl-phenoxy}-acetic acid

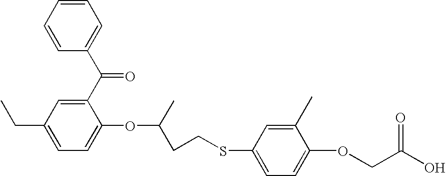

### Step A

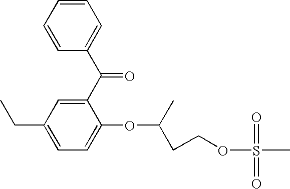

Triethyl amine (46 μL, 0.33 mmol) and methanosulfonyl chloride (24 μL, 0.30 mmol) is added to [5-ethyl-2-(3-hydroxy-1-methyl-propoxy-)phenyl]-phenyl-methanone (Example 1 Step B) (83 mg, 0.28 mmol) in DCM (1 ml) at 0° C. under N₂. The mixture is stirred for 3 h at 0° C., and HCl (1M) is added and extracted with EtOAc. Organic phase is combined, washed with saturated aqueous sodium chloride, dried over magnesium sulfate, filtered and concentrated under reduced pressure to obtain title compound (84 mg, 0.23 mmol). ES⁺ (m/e) 377.22 (M+H)⁺; ¹H NMR (400 MHz, CDCl₃) 7.77-7.79 (m, 2H), 7.53-7.57 (m, 1H), 7.41-7.45 (m, 2H), 7.26-7.28 (m, 1H), 7.20 (d, 1H, J=2.4 Hz), 6.89 (d, 1H, J=8 Hz), 4.48-4.52 (m, 1H), 4.051 (m, 2H), 2.62 (q, 2H, J=7.6 Hz), 2.87 (s, 3H), 1.68-1.93 (m, 2H), 1.22 (t, 3H, J=7.6 Hz), 1.18 (d, 3H, J=6.4 Hz).

### Step B

### {4-[3-(2-Benzoyl-4-ethyl-phenoxy)-butyl]-2-methyl-phenoxy}-acetic acid

Cesium carbonate (30 mg, 93 μmol) is added to methanosulfonic acid 2-(2-benzoyl-4-ethyl-phenoxy)-propylester (29 mg, 78 μmol) and (4-mercapto-2-methyl-phenoxy)-acetic acid ethyl ester (21.2 mg, 93 μmol) in DMF (0.6 mL), and the mixture is stirred under N₂ at 55° C. After 18 h, the mixture is cooled to ambient temperature and filtered. Solids are washed with EtOAc. The filtrate is washed with water and saturated aqueous sodium chloride, and then dried over magnesium sulfate and filtered. The organic phase is concentrated under reduced pressure. Purification by flash chromatography, silica, hexanes: EtOAc (86:14) gives {4-[3-(2-benzoyl-4-ethyl-phenoxy)-butyl]-2-methyl-phenoxy}-acetic acid ethyl ester (18 mg, 36 μmol, 45%): ES⁺ (m/e) 507.26; R_{f}=0.40 hexanes: EtOAc (80:20).

Aqueous solution of sodium hydroxide (5M, 0.07 mL, 0.35 mmol) is added to the above acetic acid ethyl ester (18 mg, 0.03 mmol) in ethanol (0.6 mL) and stirred at ambient temperature for 3 h. The mixture is acidified to pH=2 with a 1 M aqueous solution of HCl and extracted with EtOAc. The organic layers are combined and washed with saturated aqueous sodium chloride, dried over magnesium sulfate, filtered and concentrated under reduced pressure to afford the title compound as a colorless oil (16 mg, 0.03 mmol, 100%): ES⁺ (m/e) 479.22 (M+H)⁺, 501.20 (M+Na)⁺.

### EXAMPLE 10

### 3-{4-[3-(2-Benzoyl-4-ethyl-phenoxy)-butylsulfanyl]-2-methyl-phenyl}-propionic acid

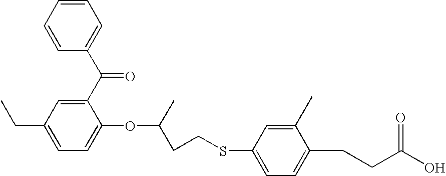

Cesium carbonate (40 mg, 123 μmol) is added to methanosulfonic acid 2-(2-benzoyl-4-ethyl-phenoxy)-propylester (Example 9, Step A) (39 mg, 102 μmol) and 3-{4-mercapto-2-methyl-phenyl)-propionic acid methyl ester (26 mg, 123 μmol) in DMF (0.7 mL), and the mixture is stirred under N₂ at 55° C. After 18 h, the mixture is cooled to ambient temperature and filtered. Solid is washed with EtOAc. Filtrate is washed with water and saturated aqueous sodium chloride, and then dried over magnesium sulfate and filtered. The organic phase is concentrated under reduced pressure. Purification by flash chromatography, silica, hexanes: EtOAc (85:15) provides 3-{4-[3-(2-benzoyl-4-ethyl-phenoxy)-butylsulfanyl]-2-methyl-phenyl}-propionic acid methyl ester (32 mg, 65 μmol, 64%): ES⁺ (m/e) 491.26; R_{f}=0.36 hexanes: EtOAc (80:20).

Aqueous solution of sodium hydroxide (5M, 0.13 mL, 0.64 mmol) is added to the above propionic acid methyl ester (32 mg, 0.06 mmol) in methanol (0.7 mL), and the mixture is stirred at ambient temperature for 3 h. The mixture is acidified to pH 2 with a 1 M aqueous solution of HCl and extracted with EtOAc. Organic layers are combined, washed with saturated aqueous sodium chloride, dried over magnesium sulfate, filtered and concentrated under reduced pressure to give title compound as a colorless oil (30 mg, 0.06 mmol, 100%): ES⁺ (m/e) 477.24 (M+H)⁺.

### EXAMPLE 11

### 2-{4-[3-ethyl-2-isobutyryl-phenoxy)-butoxy]-phenoxy}-2-methyl-propionic acid

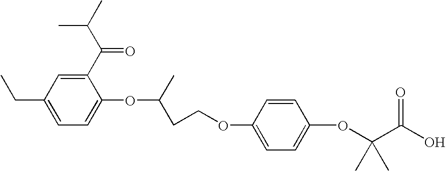

### Step A

### 1-(5-ethyl-2-hydroxy-phenyl)-2-methyl-propan-1-one

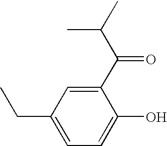

Aluminum chloride (0.35 g, 2.6 mmol) is added in portions to p-ethylanisole (0.30 g, 2.2 mmol) in DCM (2.2 mL) at 0° C. under N₂. After stirring the mixture for 10 min., isobutyryl chloride (0.25 mL, 2.4 mmol) is added dropwise. The mixture is stirred at 0° C. for 4 h and then poured in ice. The mixture is warmed to ambient temperature and then extracted with EtOAc. Organic layers are combined, washed with aqueous sodium chloride, dried over magnesium sulfate, filtered and concentrated under reduced pressure to obtain a yellow oil. The crude mixture is dissolved in toluene (2.6 mL), and aluminum chloride (0.29 g, 2.2 mmol) is added in portions at ambient temperature, and then stirred under N₂. The mixture is warmed at 80° C. for 3 h and for 16 h at 55° C. The mixture is cooled to ambient temperature and poured in ice. The mixture is extracted with EtOAc. Organic phase is combined and washed with aqueous sodium chloride, and then dried over magnesium sulfate, filtered and concentrated under reduced pressure. Purification by flash silica gel chromatography, eluting with hexanes: EtOAc (97:3) provides the title compound as a yellow oil (0.35 g, 1.82 mmol, 83%): ES⁺ (m/e) 193.16 (M+H)⁺, R_{f}=0.37 hexanes:EtOAc (90:10).

### Step B

### 2-{4-[3-ethyl-2-isobutyryl-phenoxy)-butoxy]-phenoxy}-2-methyl-propionic acid

Cesium carbonate (96 mg, 0.29 mmol) is added to 1-(5-ethyl-2-hydroxy-phenyl)-2-methyl-propan-1-one (56 mg, 0.29 mmol) and 2-[4-(3-methanesulfonyloxy-butoxy)-phenoxy]-2-methyl-propionic acid ethyl ester (100 mg, 0.26 mmol) in DMF (1 mL), and the mixture is stirred under N₂ at 55° C. After 16 h, the mixture is cooled to ambient temperature and filtered. The solid is washed with EtOAc. The filtrate is washed with water and saturated aqueous sodium chloride, and then dried over magnesium sulfate, filtered and concentrated organic phase under reduce pressure. Purification by flash chromatography, silica, hexanes: EtOAc (85:15) provides 2-{4-[3-ethyl-2-isobutyryl-phenoxy)-butoxy]-phenoxy}-2-methyl-propionic acid ethyl ester (40 mg, 0.12 mmol, 44%): ES⁺ (m/e) 471.37 (M+H)⁺, R_{f}=0.32 hexanes: EtOAc (80:20).

Aqueous solution of sodium hydroxide (5M, 0.24 mL, 1.2 mmol) is added to the above propionic acid ethyl ester (28 mg, 0.06 mmol) in ethanol (0.8 mL), and the mixture is stirred at ambient temperature for 3 h. The mixture is acidified to pH=2 with a 1 M aqueous solution of HCl and extracted with EtOAc. Organic layers are combined, washed with saturated aqueous sodium chloride, dried over magnesium sulfate, filtered and concentrated under reduced pressure to give the title compound as a colorless oil (26 mg, 0.06 mmol, 100%): ES⁺ (m/e) 443.34 (M+H)⁺, 465.32 (M+Na)⁺.

### EXAMPLE 12

### 3-{4-[3-Ethyl-2-isobutyryl)-phenoxy]-2-methyl-phenyl}-propionic acid

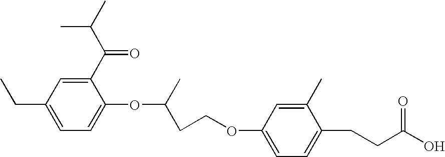

The compound of 3-{4-[3-ethyl-2-isobutyryl)-phenoxy]-2-methyl-phenyl}-propionic acid methyl ester (77 mg, 0.17 mmol, 51%) is prepared according to the procedure described in Example 11 using cesium carbonate (113 mg, 0.34 mmol), 1-(5-ethyl-2-hydroxy-phenyl)-2-methyl-propan-1-one (66 mg, 0.34 mmol) and 3-[4-(3-ethanesulfonyloxy-butoxy)-phenyl]-propionic acid methyl ester (100 mg, 0.29 mmol) in DMF (1.1 mL). ES⁺ (m/e) 441.39 (M+H)⁺, R_{f}=0.30 hexanes: EtOAc (80:20). Work up of the above propionic acid methyl ester (91 mg, 0.21 mmol) in methanol (1.5 mL) as described in Example 11, Step B provides the title compound as a colorless oil (89 mg, 0.21 mmol, 100%). ES⁺ (m/e) 427.34 (M+H)⁺.

### EXAMPLE 13

### 2-{4-[3-(2-cyclohexanecarbonyl-4-ethyl-phenoxy)-butoxy]-phenoxy-2-methyl-propionic acid

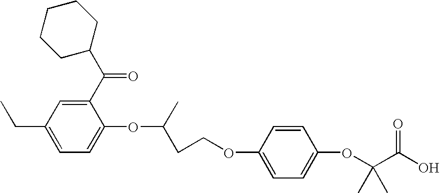

### Step A

### Cyclohexyl-(5-ethyl-2-hydroxy-phenyl)-methanone

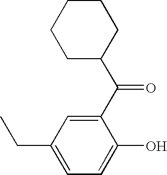

Aluminum chloride (0.35 g, 2.6 mmol) is added in portions to p-ethylanisole (0.30 g, 2.2 mmol) in DCM (2.2 mL) at 0° C. under N₂. After stirring the mixture for 10 min., cyclohexanecarbonyl chloride (0.32 mL, 2.4 mmol) is added dropwise. The mixture is stirred at 0° C. for 4 h and poured in ice. The mixture is warmed to ambient temperature and extracted with EtOAc. Organic layers are combined, washed with aqueous sodium chloride, dried over magnesium sulfate, filtered and concentrated to obtain a yellow oil. The crude mixture is dissolved in toluene (2.6 mL) and aluminum chloride (0.29 g, 2.2 mmol) is added in portions at ambient temperature. The mixture is stirred under N₂, and warmed at 80° C. for 3 h and for 16 h at 55° C. The mixture is cooled to ambient temperature and poured in ice. It is extracted with EtOAc, and organic phase is combined, washed with aqueous sodium chloride, dried over magnesium sulfate, filtered and concentrated under reduced pressure. Purification by flash silica gel chromatography, eluting with hexanes: EtOAc (97:3) provides the title compound as a yellow oil: ES⁺ (m/e) 233.15 (M+H)⁺, R_{f}=0.68 hexanes: EtOAc (90:10).

### Step B

### 2-{4-[3-(2-cyclohexanecarbonyl-4-ethyl-phenoxy)-butoxy]-phenoxy-2-methyl-propionic acid

Cesium carbonate (96 mg, 0.29 mmol) is added to cyclohexyl-(5-ethyl-2-hydroxy-phenyl)-methanone (68 mg, 0.29 mmol) and 2-[4-(3-methanesulfonyloxy-butoxy)-phenoxy]-2-methyl-propionic acid ethyl ester (100 mg, 0.26 mmol) in DMF (1 mL), stir under N₂ at 55° C. After 16 h, the mixture is cooled to ambient temperature, filtered and washed solid with EtOAc. The filtrate is washed with water and saturated aqueous sodium chloride, dried over magnesium sulfate and filtered. The organic phase is concentrated under reduce pressure. Purification by flash chromatography, silica, hexanes: EtOAc (90:10) provides 2-{4-[3-(2-cyclohexanecarbonyl-4-ethyl-phenoxy)-butoxy]-phenoxy-2-methyl-propionic acid ethyl ester (43 mg, 0.09 mmol, 32%): ES⁺ (m/e) 511.35 (M+H)⁺, R_{f}=0.45 hexanes: EtOAc (80:20). Work up of the above propionic acid ethyl ester (43 mg, 0.09 mmol) in ethanol (0.8 mL) as described in Example 11, Step B provides the title compound as a colorless oil (41 mg, 0.09 mmol, 100%): ES⁺ (m/e) 483.33 (M+H)⁺, 505.32 (M+Na)⁺.

### EXAMPLE 14

### 3-{4-[3-(2-Cyclopentanecarbonyl-4-ethyl-phenoxy)-butoxy]-2-methyl-phenyl}-propionic acid

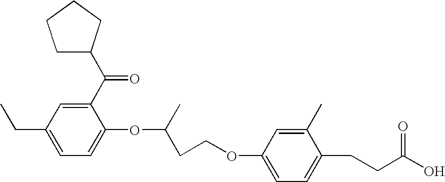

### Step A

### Cyclopentyl-(5-ethyl-2-hydroxy-phenyl)-methanone

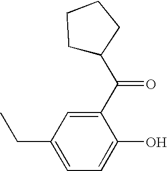

The above compound is prepared by following the procedure described in Step A, Example 13 using aluminum chloride (0.35 g, 2.6 mmol), p-ethylanisole (0.30 g, 2.2 mmol) in DCM (2.2 mL) and cyclopentylcarbonyl chloride (0.29 mL, 2.4 mmol). ES⁺ (m/e) 219.13 (M+H)⁺, R_{f}=0.72 hexanes: EtOAc (90:10).

### Step, B

### 3-{4-[3-(2-Cyclopentanecarbonyl-4-ethyl-phenoxy)-butoxy]-2-methyl-phenyl}-propionic acid

The compound of 3-{4-[3-(2-cyclopentanecarbonyl-4-ethyl-phenoxy)-butoxy]-2-methyl-phenyl}-propionic acid methyl ester (34 mg, 0.07 mmol, 43%) is prepare by following the procedure described in Example 13, Step B by using cesium carbonate (66 mg, 0.20 mmol), cyclopentyl-(5-ethyl-2-hydroxy-phenyl)-methanone (36 mg, 0.17 mmol) and 3-[4-(3-methanesulfonyloxy-butoxy)-phenyl]-propionic acid methyl ester (70 mg, 0.20 mmol) in DMF (0.8 mL). ES⁺ (m/e) 467.33 (M+H)⁺, R_{f}=0.48 hexanes: EtOAc (80:20). Work up of the above propionic acid methyl ester (9 mg, 0.02 mmol) in methanol (0.3 mL) as described in Example 11, Step B provides the title compound of the propionic acid as a colorless oil (8 mg, 0.02 mmol, 100%: ES⁺ (m/e) 453.35 (M+H)⁺.

### EXAMPLE 15

### 2-{4-[3-(2-Cyclopropanecarbonyl-4-ethyl-phenoxy)-butoxy]-phenoxy}-2-methyl-propionic acid

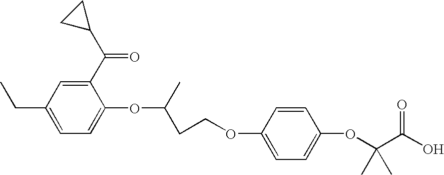

### Step A

### Cyclopropyl-(5-ethyl-2-hydroxy-phenyl)-methanone

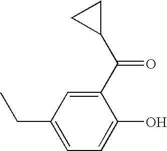

The above compound is prepared by following the procedure described in Example 13, Step A by using aluminum chloride (0.59 g, 4.4 mmol), p-ethylanisole (0.50 g, 3.7 mmol) in dichloromethane (3.6 mL) and cyclopropylcarbonyl chloride (0.36 mL, 3.9 mmol) to afford the compound as a yellow oil: ES⁺ (m/e) 191.02 (+H)⁺; R_{f}=0.49 hexanes: EtOAc (90:10).

### Step B

### 2-{4-[3-(2-Cyclopropanecarbonyl-4-ethyl-phenoxy)-butoxy]-phenoxy}-2-methyl-propionic acid

The compound of 2-{4-[3-(2-cyclopropanecarbonyl-4-ethyl-phenoxy)-butoxy]-phenoxy}-2-methyl-propionic acid ethyl ester (0.09 g, 0.19 mmol, 43%) is prepared by following the procedure described in Example 13, Step B by using cesium carbonate (0.17 g, 0.53 mmol), cyclopropyl-(5-ethyl-2-hydroxy-phenyl)-methanone (0.09 g, 0.45 mmol) and 2-[4-(3-methanesulfonyloxy-butoxy)-phenoxy]-2-methyl-propionic acid ethyl ester (0.20 g, 0.53 mmol) in DMF (2 mL). ES⁺ (m/e) 469.31 (M+H)⁺; 491.30 (M+Na)⁺. Work up of the above propionic acid ethyl ester (0.14 g, 0.32 mmol) in ethanol (2.5 mL) as described in Example 11, Step B provides the title compound as a colorless oil: ES⁺ (m/e) 441.28 (M+H)⁺, 463.26 (M+Na)⁺.

### EXAMPLE 16

### 3-{4-[3-(R)-(2-Cyclopropanecarbonyl-4-ethyl-phenoxy)-butoxy)]-2-methyl-phenyl}-propionic acid

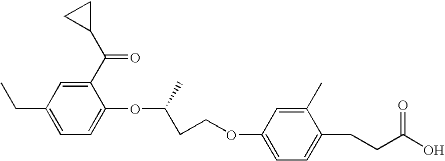

The compound of 3-{4-[3-(R)-(2-cyclopropanecarbonyl-4-ethyl-phenoxy)-butoxy)]-2-methyl-phenyl}-propionic acid methyl ester (0.14 g, 0.32 mmol, 66%) is prepared by following the procedure described in Example 13 by using cesium carbonate (0.19 g, 0.58 mmol), cyclopropyl-(5-ethyl-2-hydroxy-phenyl)-methanone (0.09 g, 0.48 mmol) and 3-[4-(3-(S)-methanesulfonyloxy-butoxy)-phenyl]-propionic acid methyl ester (0.20, 0.58 mmol) in DMF (2 mL). ES⁺ (m/e) 439.3 (M+H)⁺, 461.29 (M+Na)⁺; R_{f}=0.45 hexanes: EtOAc (80:20).

Work up of the above propionic acid methyl ester (0.14, 0.32 mmol) in methanol (2.5 mL) as described in Example 11, Step B provides the title compound as a colorless oil (0.13 g, 0.32 mmol, 100%): ES⁺ (m/e) 425.29 (+H)⁺, 447.27 (M+Na)⁺.

### EXAMPLE 17

### 3-{4-[3-(2-benzoyl-4-trifluoromethyl-phenoxy)-butoxy]-2-methyl-phenyl}-propionic acid

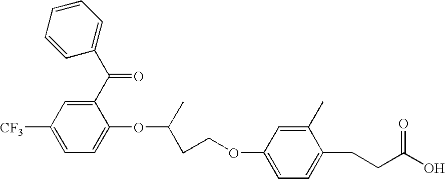

### Step A

### (2-Methoxy-5-trifluoromethyl-phenyl)-phenyl-methanone

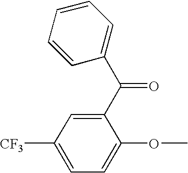

A 1.6 M solution of n-BuLi in hexanes (0.51 mL, 0.82 mmol) is added dropwise for about 20 min to N,N,N,N-tetramethylenediamine (0.12 mL, 0.80 mmol) at −20° C. under N₂. After 20 min, p-trifluoromethylanisole (0.10 g, 0.57 mmol) in THF (0.2 mL) is added dropwise for 15 min at −20° C. under N₂. After 1 h, N-methoxy-N-methyl-benzamide (0.12 mL, 0.79 mL) is added dropwise in 10 min at −20° C. under N₂. After 2 h, a 1 M HCl (0.9 mL) is added. The mixture is extracted with EtOAc, and organic phases are combined and washed with saturated aqueous sodium chloride, and then dried over magnesium sulfate, filtered and concentrated under reduced pressure. Purification by flash chromatography, silica, hexanes: EtOAc (90:10) provides the title compound (0.09 g, 0.32 mmol, 57%): ES⁺ (m/e) 281.08 (M+H)⁺; R_{f}=0.20 hexanes: EtOAc (90:10).

### Step B

### (2-Hydroxy-5-trifluoromethyl-phenyl)-phenyl-methanone

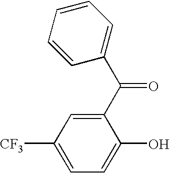

Pyridine hydrochloride (0.55 g, 4.8 mmol) is added to (2-methoxy-5-trifluoromethyl-phenyl)-phenyl-methanone (0.09 g, 0.32 mmol), and the mixture is warmed to 200° C. for 3 h under N₂. The mixture is cooled to room temperature, treated with 1 M HCl (10 mL), and then extracted with EtOAc. Organic phases are combined, washed with saturated aqueous sodium chloride, dried over magnesium sulfate, filtered and concentrated under reduced pressure. Purification by flash chromatography, silica, eluting with hexanes: EtOAc (98:2) provides the title compound (0.031 g, 0.11 mmol, 36%): ES⁻ (m/e) 265.06 (M−H)⁺; R_{f}=0.45 hexanes: EtOAc (90:10).

### Step C

### 3-{4-[3-(2-benzoyl-4-trifluoromethyl-phenoxy)-butoxy]-2-methyl-phenyl}-propionic acid

Cesium carbonate (45 mg; 0.19 mmol) is added to (2-hydroxy-5-trifluoromethyl-phenyl)-phenyl-methanone (31 mg, 0.12 mmol) and 3-[4-(3-methanesulfonyloxy-butoxy)-phenyl]-propionic acid methyl ester (48 mg, 0.14 mmol) in DMF (0.5 mL), and the mixture is stirred under N₂ at 55° C. After 16 h, the mixture is cooled to ambient temperature, and then filtered and washed solid with EtOAc. The filtrate is washed with water and saturated aqueous sodium chloride, and then dried over magnesium sulfate, filtered and concentrated under reduce pressure. Purification by flash chromatography, silica, hexanes: EtOAc (88:12) provides 3-{4-[3-(2-benzoyl-4-trifluoromethyl-phenoxy)-butoxy]-2-methyl-phenyl}-propionic acid methyl ester (33 mg, 0.06 mmol, 55%): ES⁺ (m/e) 515.26 (M+H)⁺; R_{f}=0.31 hexanes: EtOAc (80:20). Work up of the above propionic acid methyl ester (33 mg, 0.06 mmol) in methanol (0.5 mL) as described in Example 11, Step B provides the title compound as a colorless oil (30 mg, 0.06 mmol, 100%): ES⁺ (m/e) 501.24 (M+H)⁺.

### EXAMPLE 18

### 3-{4-[3-(2-Benzoyl-4-isopropyl-phenoxy)-butoxy]-2-methyl-phenyl}-propionic acid

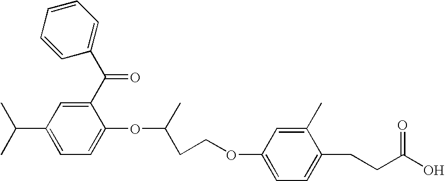

### Step A

### (2-Hydroxy-5-isopropyl-phenyl)-phenyl-methanone

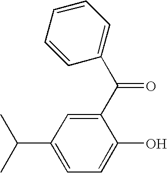

Aluminum chloride (0.32 g, 2.3 mmol) is added in portions to p-isopropylanisole (0.30 g, 1.9 mmol) in DCM (2.2 mL) at 0° C. under N₂. The mixture is stirred for 10 min and then benzoyl chloride (0.24 mL, 2.1 mmol) is added dropwise. The mixture is stirred at 0° C. for 4 h and poured in ice. The mixture is warmed to ambient temperature and extracted with EtOAc. Organic layers are combined and washed with aqueous sodium chloride, dried over magnesium sulfate, filtered and concentrated under reduced pressure to afford a yellow oil. Purification by flash chromatography, silica, hexanes: EtOAc (97:3) provides (5-isopropyl-2-methoxy-phenyl)-phenyl-methanone (0.53 g, 2.1 mmol, 100%). Pyridine hydrochloride (3.6 g, 31 mmol) is added to (5-isopropyl-2-methoxy-phenyl)-phenyl-methanone and the mixture is stirred at 200° C. for 3 h. The mixture is cooled to ambient temperature and a 1 M HCl is added. The mixture is extracted with EtOAc. Organic phases are combined, washed with a saturated solution of sodium chloride, dried over magnesium sulfate and concentrated to afford the title compound. ES⁺ (m/e) 241.02 (M+H)⁺; R_{f}=0.59 hexanes: EtOAc (9:1).

### Step B

### 3-{4-[3-(2-Benzoyl-4-isopropyl-phenoxy)-butoxy]-2-methyl-phenyl}-propionic acid

Cesium carbonate (85 mg, 0.26 mmol) is added to (2-hydroxy-5-isopropyl-phenyl)-phenyl-methanone (42 mg, 0.17 mmol) and 3-[4-(3-methanesulfonyloxy-butoxy)-phenyl]-propionic acid methyl ester (72 mg, 0.21 mmol) in DMF (0.7 mL), and the mixture is stirred under N₂ at 55° C. After 16 h, the mixture is cooled to ambient temperature, filtered and washed solid with EtOAc. The filtrate is washed with water and saturated aqueous sodium chloride, dried over magnesium sulfate, filtered and concentrated organic phase under reduce pressure. Purification by flash chromatography, silica, hexanes: EtOAc (88:12) provides 3-{4-[3-(2-Benzoyl-4-isopropyl-phenoxy)-butoxy]-2-methyl-phenyl}-propionic acid methyl ester (45 mg, 0.09 mmol, 52%): ES⁺ (m/e) 489.39 (M+H)⁺; R_{f}=0.45 hexanes: EtOAc (85:15). Work up of the above propionic acid methyl ester (31 mg, 0.06 mmol) in methanol (0.6 mL) as described Example 11, Step B provides the title compound: ES⁺ (m/e) 475.36 (M+H)⁺.

### EXAMPLE 19

### {4-[3-(R)-(2-Benzoyl-4-isopropyl-phenoxy)-butoxy]-2-methyl-phenylsulfanyl}-acetic acid

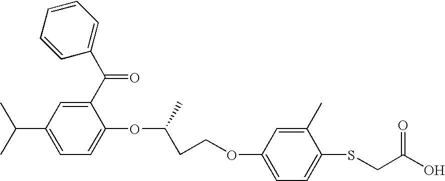

The compound of {4-[3-(R)-(2-Benzoyl-4-isopropyl-phenoxy)-butoxy]-2-methyl-phenylsulfanyl}-acetic acid ethyl ester (0.11 g, 0.21 mmol, 66%) is prepared according to Example 18 by using cesium carbonate (0.14 g, 0.43 mmol), (2-hydroxy-5-isopropyl-phenyl)-phenyl-methanone (75 mg, 0.31 mmol) and [4-(3-(S)-methanesulfonyloxy-butoxy)-2-methyl-phenylsulfanyl]acetic acid ethyl ester (0.14 g, 0.37 mmol) in DMF (1.2 mL). ES⁺ (m/e) 521.39 (M+H)⁺; R_{f}=0.35 hexanes: EtOAc (80:20). Work up of the above acetic acid ethyl ester (0.11 g, 0.21 mmol) in ethanol (1.8 mL) provides the title compound: ES⁺ (m/e) 493.30 (M+H)+.

### EXAMPLE 20

### 3-{4-[3-(R)-(2-Benzoyl-4-cyclopropyl-phenoxy)-butoxy]-2-methyl-phenyl}-propionic acid

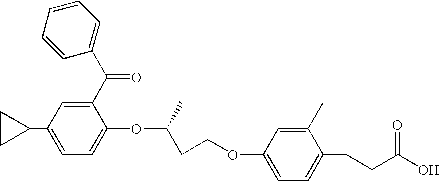

### Step A

### 1-Cyclopropyl-4-methoxy-benzene

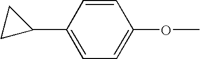

A 1 M solution of diethylzinc in hexanes (2.07 mL. 2.07 mmol) is added dropwise to a solution of 1-methoxy-4-vinyl-benzene (0.14 g, 1.03 mmol) in toluene (0.5 mL) followed by a dropwise addition of iodomethane (0.25 mL, 3.09 mmol) for 30 min. The mixture is warmed to 50° C. and the reaction is completed after about 30 min. The mixture is warmed to room temperature, diluted with water and extracted with ether. Organic phase is washed with saturated sodium chloride solution, dried over magnesium sulfate, filtered and concentrated under reduced pressure. Purification by silica flash chromatography hexanes:EtOAc (90:10) provided the title compound (0.11 g, 0.76 mmol, 74%): MS (m/e) 148 (M); R_{f}=0.60 hexanes: EtOAc (90:10).

### Step B

### (5-Cyclopropyl-2-methoxy-phenyl)-phenyl-methanone

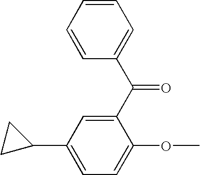

A 1.6 M solution of n-Butyl lithium in hexanes (0.63 mL, 1.0 mmol) is added dropwise for 20 min to N,N,N,N-tetramethylenediamine (0.15 mL, 0.97 mmol) in THF (0.3 mL) at −20° C. under N₂. After 20 min, 1-cyclopropyl-4-methoxy-benzene (Example 18, Step 1) (0.10 g, 0.69 mmol) in THF (1.0 mL) is added dropwise for 15 min at −20° C. under N₂. After 1 h, N-methoxy-N-methyl-benzamide (0.15 mL, 0.97 mL) is added dropwise for 10 min at −20° C. under N₂. After 2 h, a 1 M solution of aqueous HCl (0.9 mL) is added. The mixture is extracted with EtOAc, and then organic phases were combined, washed with saturated aqueous sodium chloride, dried over magnesium sulfate, filtered and concentrated under reduced pressure. Purification by flash chromatography, silica, hexanes: EtOAc (90:10) provides the title compound (0.02 g, 0.07 mmol, 27%): M (m/e) 252 (M); R_{f}=0.25 hexanes: EtOAc (90:10).

### Step C

### (5-Cyclopropyl-2-hydroxy-phenyl)-phenyl-methanone

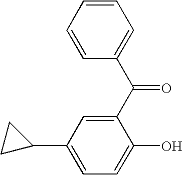

A1.6 M solution of boron tribromide (175 μL, 0.28 mmol) in DCM (1.2 mL) is added to (5-cyclopropyl-2-methoxy-phenyl)-phenyl-methanone (0.04 g, 0.16 mmol) in DCM (0.7 mL) at −78° C. under N₂. After 1 h, the mixture is cooled to 0° C. and diluted with water. Aqueous phase is extracted with additional DCM. Organic phase is washed with saturated aqueous sodium chloride, dried over magnesium sulfate, concentrated. Purification by silica flash chromatography hexanes:EtOAc (90:10) provides the title compound (0.02 g, 0.07 mol, 48%): R_{f}=0.59 hexanes: EtOAc (80:20); ¹H NMR (400 MHz, CDCl₃) 11.81 (s, 1H), 7.67-7.69 (m, 2H), 7.58-7.62 (m, 1H), 7.507.54 (m,2H), 7.31 (d, 1H, J=2.8 Hz), 7.22 (dd, 1H, J₁=2.8 Hz, J₂=8.4 Hz), 6.98 (d, 1H, 1.77-1.85 (m, 1H), 0.85-0.90 (m, 2H), 0.52-0.56 (m, 2H).

### Step D

### 3-{4-[3-(R)-(2-Benzoyl-4-cyclopropyl-phenoxy)-butoxy]-2-methyl-phenyl}-propionic acid

Cesium carbonate (38 mg, 0.17 mmol) is added to (5-cyclopropyl-2-hydroxy-phenyl)-phenyl-methanone (17 mg, 0.07 mmol) and 3-[4-(3-(S)-methanesulfonyloxy-butoxy)-phenyl]-propionic acid methyl ester (33 mg, 0.09 mmol) in DMF (0.80 mL), and the mixture is stirred under N₂ at 55° C. After 16 h, the mixture is cooled to ambient temperature and then filtered and washed solid with EtOAc. The filtrate is washed with water and saturated aqueous sodium chloride, and then dried over magnesium sulfate, filtered, and concentrated under reduce pressure. Purification by flash chromatography, silica, hexanes: EtOAc (89:11) provides 3-{4-[3-(R)-(2-benzoyl-4-cyclopropyl-phenoxy)-butoxy]-2-methyl-phenyl}-propionic acid methyl ester (19 mg, 0.04 mmol, 54%): ES⁺ (m/e) 487.16 (M+H)⁺; R_{f}=0.36 hexanes: EtOAc (80:20).

Aqueous solution of sodium hydroxide (0.12 mL, 0.59 mmol) is added to the above propionic acid methyl ester (19 mg, 0.04 mmol) in methanol (0.7 mL) and the mixture is stirred at ambient temperature for 5 h. The mixture is acidified to pH=2 with a 1 M aqueous solution of HCl, and extract with EtOAc. Organic layers are combined and washed with saturated aqueous sodium chloride, dried over magnesium sulfate, filtered and concentrated at reduced pressure to afford the title compound as a colorless oil (30 mg, 0.06 mmol, 100%): ES⁺ (m/e) 473.44 (M+H)⁺.

### EXAMPLE 21

### 3-{4-[3-(R)-(2-Benzoyl-4-chloro-phenoxy)-butoxy]-2-methyl-phenyl}-propionic acid

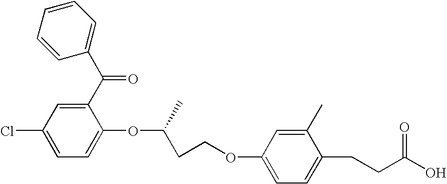

The compound of 3-{4-[3-(R)-(2-Benzoyl-4-chloro-phenoxy)-butoxy]-2-methyl-phenyl}-propionic acid methyl ester (0.41 g, 0.85 mmol, 75%) is prepared according to the procedure described in Example 20, Step D by using cesium carbonate (0.55 g, 1.69 mmol), (5-chloro-2-hydroxy)-phenyl-methanone (0.26 g, 1.13 mmol) and 3-[4-(3-(S)-methanesulfonyloxy-butoxy)-phenyl]-propionic acid methyl ester (0.47 g, 1.35 mmol) in DMF (4.8 mL). ES⁺ (m/e) 481.35 (M+H)⁺, 503.34 (M+Na)⁺; R_{f}=0.42 hexanes:EtOAc (80:20). Work up of the above propionic acid methyl ester (0.41 g, 0.85 mmol) in methanol (9 mL) as described in Example 20, Step D provides the title compound as a colorless oil (0.39 g, 0.85 mmol, 100%): ES⁺ (m/e) 467.2 (M+H)⁺, 489.2 (M+Na)⁺.

### EXAMPLE 22

### {4-[3-(R)-(2-Benzoyl-4-chloro-phenoxy)-butoxy]-2-methyl-phenylsulfanyl}-acetic acid

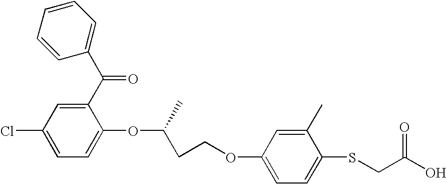

The compound of {4-[3-(R)-(2-benzoyl-4-chloro-phenoxy)-butoxy]-2-methyl-phenylsulfanyl}-acetic acid ethyl ester (0.17 g, 0.33 mmol, 77%) is prepared according to the procedure described in Example 20, Step D by using cesium carbonate (0.21 g, 0.64 mmol), (5-chloro-2-hydroxy)-phenyl-methanone (0.10 g, 0.43 mmol) and [4-(3-(S)-methanesulfonyloxy-butoxy)-2-methyl-phenylsulfanyl]acetic acid ethyl ester (0.19 g, 0.52 mmol) in DMF (1.8 mL). ES⁺ (m/e) 513.33 (M+H)⁺; R_{f}=0.46 hexanes:EtOAc (80:20). Work up of the above acetic acid ethyl ester (0.17 g, 0.33 mmol) in ethanol (3.5 mL) as described in Example 20, Step D provides the title compound as a colorless oil (0.16 g, 0.33 mmol, 100%): ES⁺ (m/e) 507.16 (M+Na)⁺.

### EXAMPLE 23

### 3-(4-{3-(R)-[4-Ethyl-2-(hydroxy-phenyl-methyl)-phenoxy]-butoxy}-2-methyl-phenyl)-propionic acid

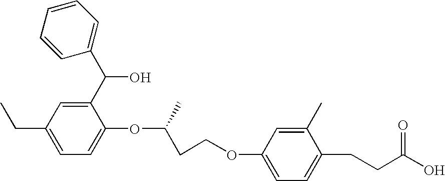

### Step A

### 3-{4-[3-(R)-(2-benzoyl-4-ethy-phenoxy)-butoxy]-2-methyl-phenyl}-propionic acid methyl ester

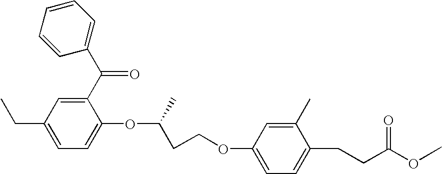

The compound of 3-{4-[3-(R)-(2-benzoyl-4-ethy-phenoxy)-butoxy]-2-methyl-phenyl}-propionic acid methyl ester (0.56 g, 1.18 mmol, 58%) is prepared according to the procedure described in Example 20, Step D by using cesium carbonate (0.82 g, 2.53 mmol), (5-ethyl-2-hydroxy-phenyl-methanone (0.38 g, 1.69 mmol) and 3-[4-(3-(S)-methanesulfonyloxy-butoxy)-phenyl]-propionic acid methyl ester (0.70 g, 2.03 mmol) in DMF (6.5 mL). ES⁺ (m/e) 475.24(M+H)⁺, 497.24(M+Na)⁺; R_{f}=0.42 hexanes:EtOAc (80:20).

### Step B

### 3-(4-{3-(R)-[4-Ethyl-2-(hydroxy-phenyl-methyl)-phenoxy]-butoxy}-2-methyl-phenyl)-propionic acid

Sodium borohydride (3.8 mg, 0.10 mmol) is added to the above propionic acid methyl ester (44 mg, 0.09 mmol) in methanol (0.50 mL) at 0° C. under N₂. The mixture is warmed to ambient temperature. After 2 h, the mixture is cooled to 0° C. and water is added. The mixture is extracted with EtOAc and organic phase is combined, washed with saturated aqueous sodium chloride, dried over magnesium sulfate, filtered and concentrate under reduced pressure. Purification by flash chromatography, silica, hexanes: EtOAc (85:15) provides 3-(4-{3-(R)-[4-ethyl-2-(hydroxy-phenyl-methyl)-phenoxy]-butoxy}-2-methyl-phenyl)-propionic acid methyl ester (31 mg, 0.07 mmol, 70%): ES⁺ (m/e) 459.48 (M−H₂O+H)⁺, 494.51 (M+Na)⁺; R_{f}=0.36 hexanes: EtOAc (80:20).

Work up of the above propionic acid methyl ester (31 mg, 0.07 mmol) in methanol (0.60 mL) as described in Example 20, Step D provides the title compound as a colorless oil (30 mg g, 0.07 mmol, 100%): ES⁻ (m/e) 461.1 (M−H)⁻, ES⁺ (m/e) 485.42 (M+H)⁺.

### EXAMPLE 24

### 3-(4-{3-(R)-[4-Ethyl-2-(hydroxyimino-phenyl-methyl)-phenoxy)-butoxy}-2-methyl-phenyl)-propionic acid

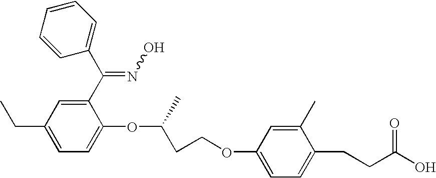

Hydroxylamine hydrochloride (26.9 mg, 0.39 mmol) is added to 3-{4-[3-(2-benzoyl-4-ethyl-phenoxy)-butoxy]-2-methyl-phenyl}-propionic acid methyl ester (Example 23, Step A) (46 mg, 0.09 mmol) in pyridine (0.3 mL) and ethanol (0.3 mL). The mixture is warmed to reflux under N₂. After 3 h, the mixture is cooled to ambient temperature and then diluted with water and extracted with EtOAc. Organic phase is combined, washed with saturated aqueous sodium chloride, dried over magnesium sulfate, filtered and concentrated under reduced pressure. Purification by flash chromatography, silica, hexanes: EtOAc (70:30) provides 3-(4-{3-(R)-[4-ethyl-2-(hydroxyimino-phenyl-methyl)-phenoxy)-butoxy}-2-methyl-phenyl)-propionic acid methyl ester (30 mg, 0.06 mmol, 63%): ES⁺ (m/e) 490.50 (M+H)⁺; R_{f}=0.26 hexanes:EtOAc (75:25). Work up of the above propionic acid methyl ester (30 mg, 0.06 mmol) in methanol (0.5 mL) as described in Example 20, Step D provides the title compound as a colorless oil (29 mg g, 0.06 mmol, 100%): ES⁺ (m/e) 476.44 (M+H)⁺.

### EXAMPLE 25

### 3-(4-{3-(R)-[4-Ethyl-2-(methoxyimino-phenyl-methyl)-phenoxy]-butoxy}-2-methyl-phenyl)-propionic acid

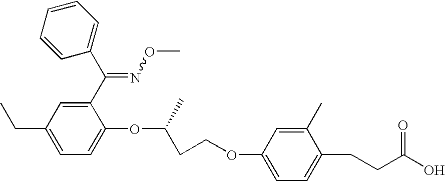

The compound of 3-(4-{3-(R)-[4-ethyl-2-(methoxyimino-phenyl-methyl)-phenoxy)-butoxy}-2-methyl-phenyl)-propionic acid methyl ester (13 mg, 0.03 mmol, 47%) is prepared according to the procedure described in Example 24 by using o-methyl-hydroxylamine hydrochloride (19 mg, 0.23 mmol), and 3-{4-[3-(2-benzoyl-4-ethyl-phenoxy)-butoxy]-2-methyl-phenyl}-propionic acid methyl ester (Example 23, Step A) (27 mg, 0.06 mmol) in pyridine (0.25 mL) and ethanol (0.25 mL). ES⁺ (m/e) 504.22 (M+H)⁺. Work up of the above propionic acid methyl ester (13 mg, 0.03 mmol) in methanol (0.40 mL) as described in Example 20, Step D provides the title compound as a colorless oil (11 mg, 0.04 mmol, 100%): ES⁺ (m/e) 490.22 (M+H)⁺.

### EXAMPLE 26

### 3-{4-[3-(Benzoyl-5-ethyl-pyridin-2-yloxy)-butoxy]-2-methyl-phenyl}-propionic acid

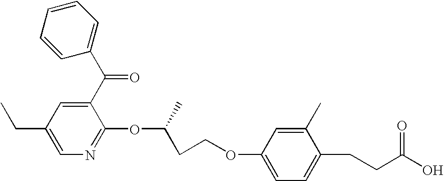

### Step A

### 5-Ethyl-2-methoxy-pyridine

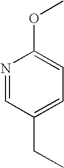

A 1.7 M solution of tert-butyllithium in pentane (16.3 mmol, 9.6 mL) is added to 5-bromo-2-methoxy-pyridine (1.50 g, 7.97) and after 1 h, ethyl iodide (1.90 mL, 23.9 mmol) is added dropwise. The mixture is warmed to ambient temperature, and after 3 h, water is added and extracted with diethyl ether. Organic phase is dried over magnesium sulfate, filtered and concentrated under reduced pressure to give the title compound as a yellow oil: R_{f}=0.39 hexanes: EtOAc (90:10); ¹H NMR (400 MHz, CDCl₃) 7.98 (d, 1H, J=2 Hz), 7.41 (dd, 1H, J₁=2 Hz, J₂=8.4 Hz), 6.67 (d, J=8.4 Hz), 3.91 (s, 3H), 2.56 (q, 2H, J=7.6 Hz), 1.21 (t, 3H, J=7.6 Hz).

### Step B

### (5-Ethyl-2-methoxy-pyridin-3-yl)-phenyl-methanol

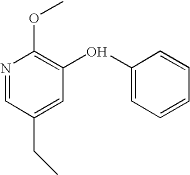

A 1.4 M solution of sec-butyllithium in cyclohexane (7.74 mL, 10.8 mmol) is added dropwise for 20 min to N,N,N,N-tetramethylenediamine (1.60 mL, 10.6 mmol) in THF (3 mL) at −78° C. under N₂ and stir. After 30 min, 5-ethyl-2-methoxy-pyridine (1.23 g, 9.68 mmol) in THF (3 mL) is added dropwise in 10 min. After 1 h, benzaldehyde (1.28 mL, 12.5 mmol) is added dropwise in 10 min. After 1 h at −78° C., the mixture is warmed to −20° C. After 90 min, water is added and the mixture is extracted with EtOAc. Organic phase is washed with aqueous saturated sodium chloride, dried over magnesium sulfate, filtered and concentrated under reduced pressure. Purification by flash chromatography, silica, hexanes: EtOAc (85:15) provides the title compound (1.5 g, 6.17 mmol, 64%): ES⁺ (m/e) 244.04 (M+H)⁺; R_{f}=0.27 hexanes: EtOAc (80:20).

### Step C

### (5-Ethyl-2-methoxy-pyridin-3-yl)-phenyl-methanone

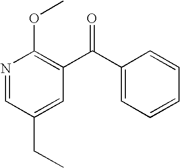

Pyridinium chlorocromate (1.73 g, 8.00 mmol) is added to 5-ethyl-2-methoxy-pyridin-3-yl)-phenyl-methanol (Example 26, Step B) (1.50 g, 6.20 mmol) in DCM (35 mL). The mixture is stirred under N₂ at ambient temperature for 2 h. The mixture is filtered through a pad of celite. Purification by flash chromatography, silica, hexanes: EtOAc (85:15) provides the title compound (0.96 g, 3.90 mmol, 64%) as a yellow oil: ES⁺ (m/e) 242.27 (M+H)⁺; R_{f}=0.48 hexanes: EtOAc (80:20).

### Step D

### (5-Ethyl-2-hydroxy-pyridin-3-yl)-phenyl-methanone

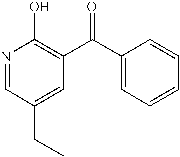

A 5.1 M solution of boron tribromide in DCM is added dropwise to (5-ethyl-2-methoxy-pyridin-3-yl)-phenyl-methanone (0.96 g, 4.0 mmol) in DCM (30 mL) at −78° C. and the mixture is stirred. The mixture is slowly warmed to ambient temperature. After 2 h, the mixture is cooled to 0° C. and water is added carefully. The mixture is extracted with EtOAc, and organic phase is combined, washed with saturated aqueous sodium chloride, dried over magnesium sulfate and concentrated under reduced pressure to give the title compound as a yellow solid (0.95 g, 4.0 mmol): ES⁺ (m/e) 228.22 (M+H)⁺.

### Step E

### 3-{4-[3-(Benzoyl-5-ethyl-pyridin-2-yloxy)-butoxy]-2-methyl-phenyl}-propionic acid

Cesium carbonate (0.46 g, 1.41 mmol) is added to 5-ethyl-2-hydroxy-pyridin-3-yl)-phenyl-methanone (0.20 g, 0.88 mmol) and 3-[4-(3-(S)-methanesulfonyloxy-butoxy)-phenyl]-propionic acid methyl ester (0.39 g, 1.14 mmol) in DMF (3.8 mL), and the mixture is stirred under N₂ at 55° C. After 16 h, the mixture is cooled to ambient temperature, and then filtered and washed solid with EtOAc. The filtrate is washed with water and saturated aqueous sodium chloride, and then dried over magnesium sulfate, filtered and concentrated under reduce pressure. Purification by flash chromatography, silica, hexanes: EtOAc (92:8) provides 3-{4-[3-(benzoyl-5-ethyl-pyridin-2-yloxy)-butoxy]-2-methyl-phenyl}-propionic acid methyl ester (0.16 g, 0.34 mmol, 38%): ES⁺ (m/e) 476.2 (M+H)⁺; R_{f}=0.35 hexanes: EtOAc (80:20).

Aqueous solution of sodium hydroxide (5M, 1.20 mL, 5.0 mmol) is added to the above propionic acid methyl ester (0.16 g, 0.34 mmol) in methanol (3 mL), and the mixture is stirred at ambient temperature for 6 h. The mixture is acidified to pH=7 with a 1 M aqueous solution of HCl and extracted with EtOAc. Organic layers are combined and washed with saturated aqueous sodium chloride, which is then dried over magnesium sulfate, filtered and concentrated under reduced pressure to afford the title compound as an oil. ES⁺ (m/e) 462.17 (M+H)⁺.

### EXAMPLE 27

### {4-[3-(3-Benzoyl-5-ethyl-pyridin-2-yloxy)-butoxy]-2-methyl-phenylsulfanyl}-acetic acid

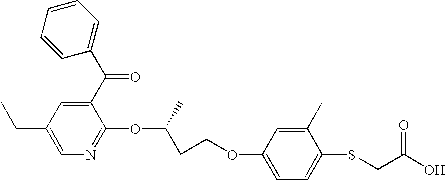

The compound of {4-[3-(3-benzoyl-5-ethyl-pyridin-2-yloxy)-butoxy]-2-methyl-phenylsulfanyl}-acetic acid ethyl ester (0.07 g, 0.14 mmol, 26%) is prepared according to the procedure described in Example 26 by using cesium carbonate (0.26 g, 0.79 mmol), 5-ethyl-2-hydroxy-pyridin-3-yl)-phenyl-methanone (Example 26, Step D) (0.12 g, 0.53 mmol) and [4-(3-(S)-methanesulfonyloxy-butoxy)-2-methyl-phenylsulfanyl]acetic acid ethyl ester (0.24 g, 0.63 mmol) in ACN (2.3 mL). ES⁺ (m/e) 508.15 (M+H)⁺; R_{f}=0.62 hexanes: EtOAc (50:50). Work up of the above acetic acid ethyl ester (0.07 g, 0.14 mmol) in ethanol (1.5 mL) as described in Example 20, Step D provides the title compound as an oil: ES⁺ (m/e) 480.15 (M+H)⁺.

### EXAMPLE 28

### 3-{4-[3-(5-Ethyl-biphenyl-2-yloxy)-butoxy]-2-methyl-phenyl}-propionic acid

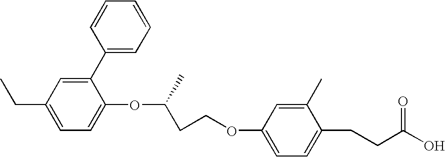

### 2-Bromo-4-ethyl-1-methoxy-benzene

### Step A

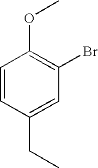

N-bromosuccinimide (0.72 g, 4.03 mmol) is added to 1-ethyl-4-methoxy-benzene (0.50 g, 3.67 mmol) in ACN (15 mL), and the mixture is stirred under N₂ at ambient temperature. After 24 h, the mixture is concentrated under reduced pressure and diluted with water. The mixture is extracted with EtOAc, and organic phases is washed with saturated aqueous sodium chloride, dried over magnesium sulfate, filtered and concentrated under reduced pressure. Purification by flash chromatography, silica, hexanes: EtOAc (95:5) provides the title compound (0.74 g, 3.44 mmol, 94%): ES⁺ (m/e) 228.92 (M (⁷⁹Br)+H)⁺, 230.85 (M (⁸¹Br)+H)⁺; ¹H NMR (400 MHz, CDCl₃) 7.38 (d, 1H, J=1.6 Hz), 7.08 (dd, 1H, J₁=1.6 Hz, J₂=8.4 Hz), 6.81 (d, 1H, J=8.4 Hz); 3.86 (s, 3H), 2.57 (q, 2H, J=7.6 Hz), 1.21 (t, 3H, J=7.6 Hz).

### Step B

### 5-Ethyl-2-methoxy-biphenyl

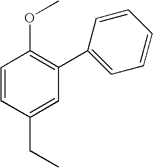

Tetrakis(triphenyl phosphine)palladium(0) (54 mg, 0.05 mmol) is added to 2-bromo-4-ethyl-1-methoxy-benzene (0.20 g, 0.94 mmol) in dimethoxyethane (3.5 mL) under N₂, and the mixture is stirred. After 10 min, phenylboronic acid (0.17 g, 1.39 mmol) and sodium carbonate (0.29 g, 2.79 mmol) in water (1.7 mL) are added. The mixture is warmed to 80° C. for 18 h and then cooled to room temperature. Water is added and the mixture is extracted with EtOAc. Organic phase is combined and washed with saturated aqueous sodium chloride. Organic phase is dried over magnesium sulfate, filtered and concentrated under reduced pressure. Purification by flash chromatography, silica, hexanes: EtOAc (85:15) provides the title compound as an oil (0.18 g, 0.85 mmol, 92%): ES⁺ (m/e) 213.08 (M+H)⁺; R_{f}=0.50 hexanes: EtOAc (90:10).

### Step C

### 5-Ethyl-biphenyl-2-ol

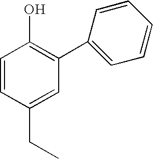

A 1.65 M solution of boron tribromide in DCM (0.86 mL, 1.41 mmol) is added to 5-ethyl-2-methoxy-biphenyl (0.1 g, 0.47 mmol) in DCM (4 mL) under N₂ at −78° C., and the mixture is stirred. The mixture is warmed to −10° C., and after 2 h, water is added and then extracted with DCM. Organic phases are combined, washed with saturated aqueous sodium chloride, dried over magnesium sulfate, filtered and concentrated under reduced pressure. Purification by flash chromatography, silica, hexanes: EtOAc (95:5) provides the title compound (0.08 g, 0.44 mmol, 93%): ES⁻ (m/e) 197.11 (M−H)⁻; R_{f}=0.18 hexanes: EtOAc (90:10).

### Step D

### 3-{4-[3-(5-Ethyl-biphenyl-2-yloxy)-butoxy]-2-methyl-phenyl}-propionic acid

Cesium carbonate (0.11 g, 0.33 mmol) is added to 5-ethyl-biphenyl-2-ol (0.04 g, 0.20 mmol) and 3-[4-(3-(S)-methanesulfonyloxy-butoxy)-phenyl]-propionic acid methyl ester (0.09 g, 0.26 mmol) in DMF (0.65 mL), and the mixture is stirred under N₂ at 55° C. After 16 h, the mixture is cooled to ambient temperature, filtered and washed solid with EtOAc. The filtrate is washed with water and saturated aqueous sodium chloride, and then dried over magnesium sulfate, filtered and concentrated under reduce pressure. Purification by flash chromatography, silica, hexanes: EtOAc (93:7) provides 3-{4-[3-(5-ethyl-biphenyl-2-yloxy)-butoxy]-2-methyl-phenyl}-propionic acid methyl ester (0.05 g, 0.11 mmol, 55%): ES⁺ (m/e) 464.3 (M+NH₄)⁺; R_{f}=0.29 hexanes: EtOAc (80:20). Work up of the above propionic acid methyl ester (0.05 g, 0.11 mmol) in methanol (1 mL) as described in Example 20, Step D provides the title compound (0.04, 0.11 mmol, 100%): ES⁺ (m/e) 433.31 (M+H)⁺, 455.28 (M+Na)⁺.

### EXAMPLE 29

### 3-(4-{3-(S)-[4-Ethyl-2-(1H-pyrrol-2-yl)-phenoxy]-butoxy}-2-methyl-phenyl-propionic acid

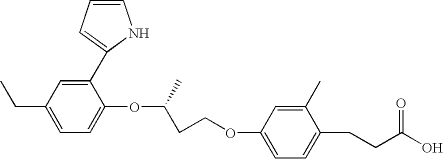

### Step A

### 2-(5-Ethyl-2-methoxy-phenyl)-pyrrole-1-carboxilic acid tert-butyl ester

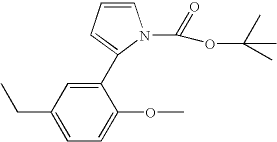

Tetrakis (triphenyl phosphine)palladium(0) (54 mg, 0.05 mmol) is added to 2-bromo-4-ethyl-1-methoxy-benzene (0.20 g, 0.93 mmol) in dimethoxyethane (3.5 mL) under N₂ and the mixture is stirred. After 10 min, N-terbutoxycarbonyl-pyrrole-2-boronic acid (0.25 g, 1.20 mmol) and sodium carbonate (0.26 g, 2.42 mmol) in water (1.7 mL) are added. The mixture is warmed to 80° C. for 18 h. The mixture is cooled to room temperature, and then water is added and extracted with EtOAc. Organic phases are combined and washed with saturated aqueous sodium chloride. Organic phases are dried over magnesium sulfate, filtered and concentrated under reduced pressure. Purification by flash chromatography, silica, hexanes: EtOAc (92:8) provides the title compound as an oil (0.21 g, 0.69 mmol, 74%): ES⁺ (m/e) 202.23 (M−COOC(CH₃)₃+2H)⁺.

### Step B

### 4-Ethyl-2-(1H-pyrrol-2-yl)-phenol

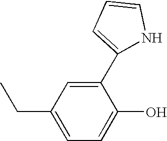

A 1.65 M solution of boron tribromide in DCM (0.63 mL, 1.0 mmol) is added to 2-(5-ethyl-2-methoxy-phenyl)-pyrrole-1-carboxilic acid tert-butyl ester (0.1 g, 0.35 mmol) in DCM (3 mL) under N₂ at −78° C., and the mixture is stirred. The mixture is warmed to 0° C. After 2 h, water is added and extracted with DCM. Organic phases are combined, washed with saturated aqueous sodium chloride, dried over magnesium sulfate, filtered and concentrated under reduced pressure. Purification by flash chromatography, silica, hexanes: EtOAc (80:20) provides the title compound (0.01 g, 0.06 mmol, 16%): ES⁺ (m/e) 188.00 (M+H)⁺; R_{f}=0.30 hexanes: EtOAc (85:15).

### Step C

### 3-(4-{3-(S)-[4-Ethyl-2-(1H-pyrrol-2-yl)-phenoxy]-butoxy}-2-methyl-phenyl-propionic acid

Cesium carbonate (23 mg, 0.07 mmol) is added to 4-ethyl-2-(1H-pyrrol-2-yl)-phenol (11 mg, 0.06 mmol) and 3-[4-(3-(S)-methanesulfonyloxy-butoxy)-phenyl]-propionic acid methyl ester (24 mg, 0.07 mmol) in DMF (0.5 mL), and the mixture is stirred under N₂ at 55° C. After 16 h, the mixture is cooled to ambient temperature, filtered and washed solid with EtOAc. The filtrate is washed with water and saturated aqueous sodium chloride, dried over magnesium sulfate, filtered and concentrated under reduce pressure. Purification by flash chromatography, silica, hexanes: EtOAc (95:5) gives 3-(4-{3-(S)-[4-Ethyl-2-(1H-pyrrol-2-yl)-phenoxy]-butoxy}-2-methyl-phenyl-propionic acid methyl ester (5 mg, 0.01 mmol, 19%): ES⁺ (m/e) 436.19 (M+H)⁺; R_{f}=0.43 hexanes: EtOAc (80:20). Work up of the above propionic acid methyl ester (5 mg, 0.01 mmol) in methanol (0.5 mL) as described in Example 20, Step D provides the title compound (3 mg, 0.01 mmol, 100%): ES⁺ (m/e) 422.2 (M+H)⁺.

### EXAMPLE 30

### 3-{4-[3-(S)-(4-Ethyl-2-thiophen-2-yl-phenoxy)-butoxy]-2-methyl-phenyl}-propionic acid

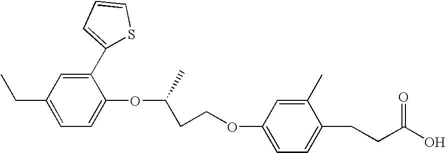

### Step A

### 2-Bromo-4-ethyl-phenol

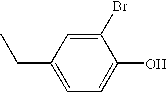

N-bromosuccinimide (1.58 g, 8.92 mmol) is added to a solution of 4-ethyl phenol (1.0 g, 8.19 mmol) in ACN (35 mL), and the mixture is stirred under N₂ at ambient temperature. After 24 h, the mixture is concentrated under reduced pressure and diluted with water. The mixture is extracted with EtOAc, and organic phases are washed with saturated aqueous sodium chloride, dried over magnesium sulfate, filtered and concentrated under reduced pressure. Purification by flash chromatography, silica, hexanes: EtOAc (95:5) yields title compound (1.01 g, 4.9 mmol, 61%): R_{f}=0.34 hexanes:EtOAc (90:10), ¹H NMR (400 MHz, CDCl₃) 7.28 (d, 1H, J=2.4 Hz), 7.03 (dd, 1H, J₁=2.4 Hz, J₂=8.4 Hz), 6.92 (d, 1H, J=8.4 Hz), 5.34 (s, 1H), 2.56 (q, 2H, J=7.6 Hz), 1.20 (t, 3H, J=7.6 Hz).

### Step B

### 4-Ethyl-2-thiophen-2-yl-phenol

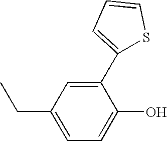

Tetrakis(triphenyl phosphine)palladium(0) (57 mg, 0.05 mmol) is to 2-bromo-4-ethyl-phenol (0.20 g, 0.99 mmol) in dimethoxyethane (3.3 mL) under N₂, and the mixture is stirred. After 10 min, 2-thiophene boronic acid (0.16 g, 1.29 mmol) and sodium carbonate (0.27 g, 2.57 mmol) in water (1.6 mL) are added. The mixture is warmed to 80° C. for 18 h. The mixture is cooled to room temperature and then water is added and extracted with EtOAc. Organic phases are combined and washed with saturated aqueous sodium chloride. Organic phases are dried over magnesium sulfate, filtered and concentrated under reduced pressure. Purification by flash chromatography, silica, hexanes: EtOAc (90:10) provides the title compound as an oil (0.08 g, 0.39 mmol, 40%): R_{f}=0.44 hexanes: EtOAc (90:10), ¹H NMR (400 MHz, CDCl₃) 7.39 (dd, 1H, J₁=1.6 Hz, J₂=5.4 Hz), 7.28 (dd, 1H, J₁=1.6, J₂=3.4 Hz), 7.23 (d, 1H, J=2.4 Hz), 7.14 (dd, 1H, J₁=3.4, J₂=5.4 Hz, 7.07 (dd, 1H, J₁=2.4 Hz, J₂=8.4 Hz), 6.89 (d, 1H, J=8.4 Hz), 5.32 (s, 1H), 2.61 (q, 2H, J=7.6 Hz), 1.24 (t, 3H, J=7.6 Hz).

### Step C

### 3-{4-[3-(S)-(4-Ethyl-2-thiophen-2-yl-phenoxy)-butoxy]-2-methyl-phenyl}-propionic acid

Cesium carbonate (0.13 g, 0.40 mmol) is added to 4-ethyl-2-thiophen-2-yl-phenol (51 mg, 0.25 mmol) and 3-[4-(3-(S)-methanesulfonyloxy-butoxy)-phenyl]-propionic acid methyl ester (0.10, 0.30 mmol) in DMF (1.4 mL), and the mixture is stirred under N₂ at 55° C. After 16 h, the mixture is cooled to ambient temperature and filtered. The solids are washed with EtOAc. The filtrate is washed with water and saturated aqueous sodium chloride, and then dried over magnesium sulfate, filtered and concentrated under reduce pressure. Purification by flash chromatography, silica, hexanes: EtOAc (95:5) provides 3-{4-[3-(S)-(4-ethyl-2-thiophen-2-yl-phenoxy)-butoxy]-2-methyl-phenyl}-propionic acid methyl ester (60 mg, 0.13 mmol, 53%): ES⁺ (m/e) 453.25 (M+H)⁺; R_{f}=0.26 hexanes: EtOAc (95:5). Work up of the above propionic acid methyl ester (60 mg, 0.13 mmol) in methanol (1.0 mL) as described in Example 20, Step D provides the title compound (57 mg, 0.13 mmol, 100%): ES⁺ (m/e) 439.35 (M+H)⁺.

### EXAMPLE 31

### {4-[3-(S)-(4-Ethyl-2-thiazol-2-yl-phenoxy)-butoxy]-2-methyl-phenoxy}-propionic acid

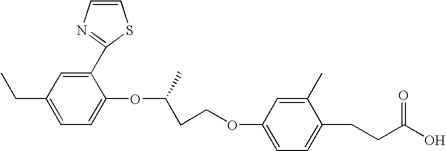

### Step A

### 4-Ethyl-2-thiazol-2-yl-phenol

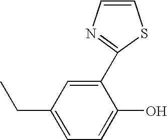

Tetrakis(triphenyl phosphine)palladium(0) (25 mg, 0.02 mmol) is added to 2-bromo-thiazole (38 μL, 0.43 mmol) in dimethoxyethane (1.4 mL) under N₂, and the mixture is stirred. After 10 min, 2-methoxy-5-ethylbenzeneboronic acid (0.10 g, 0.56 mmol) and sodium carbonate (0.12 g, 1.10 mmol) in water (0.7 mL) are added. The mixture is warmed to 95° C. for 18 h. The mixture is cooled to room temperature, and water is added and extract with EtOAc. Organic phases are combined and washed with saturated aqueous sodium chloride. Organic phases are dried over magnesium sulfate, filtered and concentrated under reduced pressure. Purification by flash chromatography, silica, hexanes: EtOAc (93:7) provides 2-(5-ethyl-2-methoxy-phenyl)-thiazole compound as an oil (0.07 g, 0.30 mmol, 55%): ES⁺ (m/e) 220.25 (M+H)⁺; R_{f}=0.30 hexanes: EtOAc (90:10).

A 4.1 M solution of boron tribromide in DMF (0.15 mL, 0.60 mmol) is added to 2-(5-ethyl-2-methoxy-phenyl)-thiazole (0.08 g, 0.30 mmol) in DCM (0.7 mL) under N₂ at −78° C., and the mixture is stirred. The mixture is warmed to 0° C. After 3 h, water is added and extracted with DCM. Organic phases are combined, washed with saturated aqueous sodium chloride, and then dried over magnesium sulfate, filtered and concentrated under reduced pressure. Purification by flash chromatography, silica, hexanes: EtOAc (98:2) provides the title compound (0.02 g, 0.11 mmol, 37%): ES⁺ (m/e) 206.18 (M+H)⁺; R_{f}=0.51 hexanes: EtOAc (90:10).

### Step B

### {4-[3-(S)-(4-Ethyl-2-thiazol-2-yl-phenoxy)-butoxy]-2-methyl-phenoxy}-propionic acid

Cesium carbonate (58 mg g, 0.18 mmol) is added to 4-ethyl-2-thiazol-2-yl-phenol (23 mg, 0.11 mmol) and 3-[4-(3-(S)-methanesulfonyloxy-butoxy)-phenyl]-propionic acid methyl ester (50 mg, 0.14 mmol) in DMF (0.7 mL), and the mixture is stirred under N₂ at 55° C. After 16 h, the mixture is cooled to ambient temperature, filtered and solid is washed with EtOAc. The filtrate is washed with water and saturated aqueous sodium chloride, and then dried over magnesium sulfate, filtered and concentrated under reduce pressure. Purification by flash chromatography, silica, hexanes: EtOAc (90:10) gives {4-[3-(S)-(4-ethyl-2-thiazol-2-yl-phenoxy)-butoxy]-2-methyl-phenoxy}-propionic acid methyl ester (37 mg, 0.08 mmol, 73%): ES⁺ (m/e) 454.40(M+H)⁺; R_{f}=0.24 hexanes: EtOAc (80:20). Work up of the above propionic acid methyl ester (37 mg, 0.08 mmol) in methanol (0.7 mL) as described in Example 20, Step D provides the title compound (35 mg, 0.08 mmol, 98%): ES⁺ (m/e) 440.34 (M+H)⁺.

### EXAMPLE 32

### 3-{4-[3-(S)-(4-Ethyl-2-thiazol-4-yl-phenoxy)-butoxy]-2-methyl-phenyl}-propionic acid

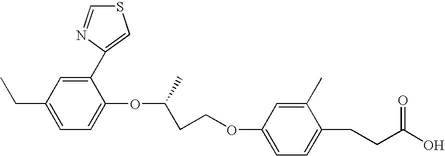

Cesium carbonate (101 mg, 0.31 mmol) is added to 4-ethyl-2-thiazol-4-yl-phenol (40 mg, 0.19 mmol) and 3-[4-(3-(S)-methanesulfonyloxy-butoxy)-phenyl]-propionic acid methyl ester (87 mg, 0.25 mmol) in DMF (1.2 mL), and the mixture is stirred under N₂ at 55° C. After 24 h, the mixture is cooled to ambient temperature and filtered. The solid is with washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (91:9) gives 3-{4-[3-(S)-(4-Ethyl-2-thiazol-4-yl-phenoxy)-butoxy]-2-methyl-phenyl}-propionic acid methyl ester (64 mg, 0.14 mmol, 73%): ES⁺ (m/e) 454.43(M+H)⁺; R_{f}=0.33 hexanes:ethyl acetate (80:20).

A 5 M aqueous solution of sodium hydroxide (0.42 mL, 2.11 mmol) is added to the above propionic acid methyl ester (64 mg, 0.14 mmol) in methanol (1.2 mL), and the mixture is stirred at ambient temperature for 9 h. The mixture is acidified to pH 2 with a 1M HCl and extracted with ethyl acetate. Organic layers are combined and washed with saturated aqueous sodium chloride, dried over magnesium sulfate, filtered and concentrated at reduced pressure to afford title compound (60 mg, 0.13 mmol, 98%): ES⁺ (m/e) 440.28 (M+H)⁺.

### EXAMPLE 33

### 3-{4-[3-(S)-(4-Ethyl-2-furan-2-yl-phenoxy)-butoxy]-2-methyl-phenyl}-propionic acid

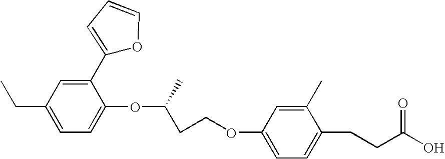

Cesium carbonate (110 mg, 0.34 mmol) is added to 4-ethyl-2-furan-2-yl-phenol (40 mg, 0.21 mmol) and 3-[4-(3-(S)-methanesulfonyloxy-butoxy)-phenyl]-propionic acid methyl ester (95 mg, 0.27 mmol) in DMF (1.2 mL), and the mixture is stirred under N₂ at 55° C. After 24 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (90:10) gives 3-{4-[3-(S)-(4-Ethyl-2-furan-2-yl-phenoxy)-butoxy]-2-methyl-phenyl}-propionic acid methyl ester (62 mg, 0.14 mmol, 66%): ES⁺ (m/e) 437.36 (M+H)⁺; R_{f}=0.37 hexanes:ethyl acetate (80:20).

A 5 M aqueous solution of sodium hydroxide (0.42 mL, 2.11 mmol) is added to 3-{4-[3-(S)-(4-ethyl-2-furan-2-yl-phenoxy)-butoxy]-2-methyl-phenyl}-propionic acid (62 mg, 0.14 mmol) in methanol (1.3 mL), and the mixture is stirred at ambient temperature for 9 h. The mixture is acidified to pH=2 with a 1 M aqueous solution of HCl and extracted with ethyl acetate. Organic layers are combined, washed with saturated aqueous sodium chloride, dried over magnesium sulfate, filtered and concentrated at reduced pressure to obtain title compound (60 mg, 0.13 mmol, 98%): ES⁺ (m/e) 423.33 (M+H)⁺.

### EXAMPLE 34

### 3-{4-[3-(S)-(4-Ethyl-2-thiophen-3-yl-phenoxy)-butoxy]-2-methyl-phenyl}-propionic acid

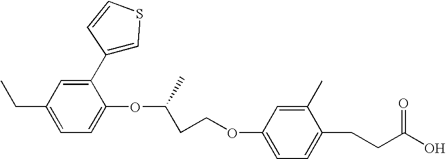

### Step A

### 4-Ethyl-2-thiophen-3-yl-phenol

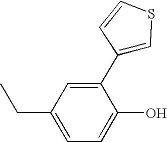

Tetrakis (triphenyl phosphine)palladium(0) (28 mg, 0.02 mmol) is added to 2-bromo-4-ethyl-phenol (0.10 g, 0.49 mmol) in dimethoxyethane (1.6 mL) under N₂ and the mixture is stirred. After 10 min, 3-thiophene boronic acid (0.08 g, 0.65 mmol) and sodium carbonate (0.14 g, 1.29 mmol) in water (0.8 mL) are added. The mixture is warmed to 80° C. for 18 h. The mixture is cooled to room temperature, and water is added. The mixture is extracted with EtOAc. Organic phases are combined and washed with saturated aqueous sodium chloride. Organic phase is dried over magnesium sulfate, filtered and concentrated under reduced pressure. Purification by flash chromatography, silica, hexanes: EtOAc (90:10) gives title compound as an oil (0.05 g, 0.21 mmol, 43%): R_{f}=0.40 hexanes: EtOAc (90:10), ¹H NMR (400 MHz, CDCl₃); ES⁺ (m/e) 205.10(M+H)⁺.

### Step B

### 3-{4-[3-(S)-(4-Ethyl-2-thiophen-3-yl-phenoxy)-butoxy]-2-methyl-phenyl}-propionic acid

The compound of 3-{4-[3-(S)-(4-Ethyl-2-thiophen-3-yl-phenoxy)-butoxy]-2-methyl-phenyl}-propionic acid methyl ester (62 mg, 0.14 mmol, 64%) is prepared according to the procedure described in Example 31, Step B by using cesium carbonate (97 mg, 0.30 mmol), 4-ethyl-2-thiophen-3-yl-phenol (44 mg, 0.21 mmol) and 3-[4-(3-(S)-methanesulfonyloxy-butoxy)-phenyl]-propionic acid methyl ester (88 mg, 0.26 mmol) in DMF (1.0 mL). ES⁺ (m/e) 437.36 (M+H)⁺; R_{f}=0.36 hexanes: EtOAc (90:10). Work up of the above propionic acid methyl ester (62 mg, 0.14 mmol) in methanol (1.0 mL) provides the title compound (60 mg, 0.13 mmol, 98%): ES⁺ (m/e) 439.26 (M+H)⁺.

### EXAMPLE 35

### 3-{4-3-(S)-(4-Ethyl-2-oxazol-4-yl-phenoxy)-butoxy]-2-methyl-phenyl}-propionic acid

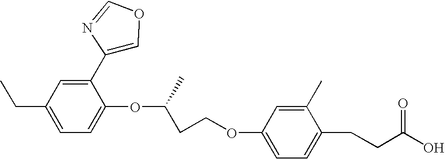

Cesium carbonate (72 mg, 0.22 mmol) is added to 4-ethyl-2-oxazol-4-yl-phenol (30 mg, 0.16 mmol) and 3-[4-(3-(S)-methanesulfonyloxy-butoxy)-phenyl]-propionic acid methyl ester (65 mg, 0.19 mmol) in DMF (0.8 mL), and the mixture was stirred under N₂ at 55° C. After 24 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (91:9) gives 3-{4-3-(S)-(4-ethyl-2-oxazol-4-yl-phenoxy)-butoxy]-2-methyl-phenyl}-propionic acid methyl ester (38 mg, 0.87 mmol, 56%): ES⁺ (m/e) 438.30 (M+H)⁺; R_{f}=0.30 hexanes:ethyl acetate (80:20).

A 5 M aqueous solution of sodium hydroxide (0.26 mL, 1.30 mmol) is added to the above propionic acid methyl ester (38 mg, 0.87 mmol) in methanol (0.7 mL), and the mixture is stirred at ambient temperature for 9 h. The mixture is acidified to pH 2 with a 1M aqueous solution of HCL and extracted with ethyl acetate. Organic layers are combined, washed with saturated wash with saturated aqueous sodium chloride, dried, over magnesium sulfate, filtered and concentrated at reduced pressure to obtain title compound (34 mg, 0.82 mmol, 95%): ES⁺ (m/e) 424.27 (M+H)⁺.

### EXAMPLE 36

### 3-{4-[3-(S)-(4-Ethyl-2-oxazol-2-yl-phenoxy)-butoxy]-2-methyl-phenyl}-propionic acid

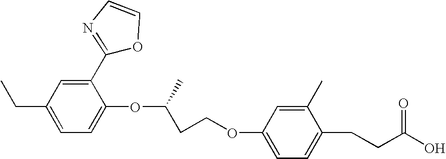

Cesium carbonate (39 mg, 0.12 mmol) is added to 4-ethyl-2-oxazol-2-yl-phenol (16 mg, 0.08 mmol) and 3-[4-(3-(S)-methanesulfonyloxy-butoxy)-phenyl]-propionic acid methyl ester (35 mg, 0.10 mmol) in DMF (0.7 mL), and the mixture is stirred under N₂ at 55° C. After 24 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (80:20) gives 3-{4-[3-(S)-(4-ethyl-2-oxazol-2-yl-phenoxy)-butoxy]-2-methyl-phenyl}-propionic acid methyl ester (14 mg, 0.03 mmol, 37%): ES⁺ (m/e) 438.35 (M+H)⁺; R_{f}=0.23 hexanes:ethyl acetate (70:30).

A 5 M aqueous solution of sodium hydroxide (0.13 mL, 0.63 mmol) is added to the above propionic acid methyl ester (13 mg, 0.03 mmol) in methanol (0.3 mL) and the mixture is stirred at ambient temperature for 9 h. The mixture is acidified to pH=2 with a 1M aqueous solution of hydrochloric acid and extracted with ethyl acetate. Organic layers are combined, washed with saturated aqueous sodium chloride, dried over magnesium sulfate, filtered and concentrated at reduced pressure to obtain title compound (10 mg, 0.02 mmol, 95%): ES⁺ (m/e) 424.31 (M+H)⁺.

### EXAMPLE 37

### 3-{4-[3-(S)-(4-Chloro-2-thiazol-4-yl-phenoxy)-butoxy]-2-methyl-phenyl}-propionic acid

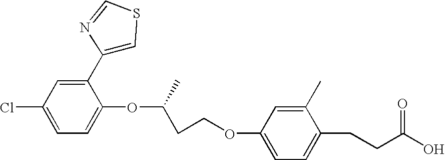

Cesium carbonate (107 mg, 0.33 mmol) is added to 4-chloro-2-thiazol-4-yl-phenol (50 mg, 0.24 mmol) and 3-[4-(3-(S)-methanesulfonyloxy-butoxy)-phenyl]-propionic acid methyl ester (97 mg, 0.28 mmol) in DMF (0.8 mL), and the mixture is stirred under N₂ at 55° C. After 24 h, a 5 M aqueous solution of NaOH (1 mL) is added, and the mixture is cooled to ambient temperature for 5 h. The mixture is acidified to pH=2 with a 1M HCl and extracted with EtOAc. Organic layers are combined and washed with saturated aqueous sodium chloride, dried over magnesium sulfate, filtrated and concentrated at reduced pressure. Oil is purified in HTC to obtain title compound (43 mg, 0.96 mmol, 41%): ES⁺ (m/e) 445.90 (M+H)⁺.

### EXAMPLE 38

### 3-{4-[3-(S)-(4-Ethyl-2-pyridin-2-yl-phenoxy)-butoxy]-2-methyl-phenyl}-propionic acid

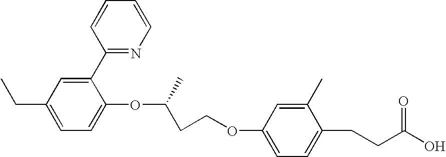

Cesium carbonate (115 mg, 0.35 mmol) is added to 4-ethyl-2-pyridin-2-yl-phenol (50 mg, 0.25 mmol) and 3-[4-(3-(S)-methanesulfonyloxy-butoxy)-phenyl]-propionic acid methyl ester (103 mg, 0.30 mmol) in DMF (0.7 mL), and the mixture is stirred under N₂ at 55° C. After 24 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (87:13) gives 3-{4-[3-(S)-(4-ethyl-2-pyridin-2-yl-phenoxy)-butoxy]-2-methyl-phenyl}-propionic acid methyl ester (64 mg, 0.14 mmol, 67%): ES⁺ (m/e) 448.56 (M+H)⁺; R_{f}=0.20 hexanes:ethyl acetate (80:20).

A 5 M aqueous solution of sodium hydroxide (0.43 mL, 2.14 mmol) is added to the above propionic acid methyl ester (64 mg, 0.14 mmol) in methanol (1.1 mL), and the mixture is stirred at ambient temperature for 9 h. The mixture is neutralized to pH=7 with a 1 M HCl and extracted with ethyl acetate. Organic layers are combined and washed with saturated aqueous sodium chloride, dried over magnesium sulfate, filtered and concentrated at reduced pressure to obtain title compound (60 mg, 0.13 mmol, 95%): ES⁺ (m/e) 434.45 (M+H)⁺.

### EXAMPLE 39

### 3-{4-[3-(S)-(4-Ethyl-2-pyridin-3-yl-phenoxy)-butoxy]-2-methyl-phenyl}-propionic acid

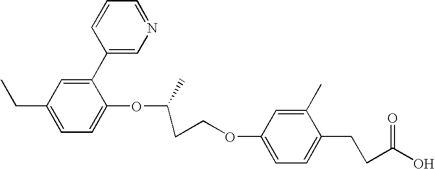

### Step A

### 4-Ethyl-2-pyridin-3-yl-phenol

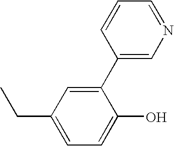

Tetrakis(triphenylphosphine)palladium(0) (57 mg, 0.05 mmol) is added to 2-bromo-4-ethyl-phenol (0.20 g, 0.99 mmol) in dimethoxyethane (3.3 mL) under N₂, and the mixture is stirred. After 10 min, pyridin-3-yl-boronic acid (0.16 g, 1.29 mmol) and sodium carbonate (0.27 g, 2.59 mmol) in water (1.6 mL) are added. The mixture is warmed to 80° C. for 18 h. The mixture is cooled to room temperature, and water is added and then extracted with EtOAc. Organic phase is combined and washed with saturated aqueous sodium chloride. Organic phase is dried over magnesium sulfate, filtered and concentrated under reduced pressure. Purification by flash chromatography, silica, hexanes: EtOAc (70:30) gives title compound as an oil R_{f}=0.20 hexanes: EtOAc (50:50); ES⁺ (m/e) 200.19 (M+H)⁺.

### Step B

### 3-{4-[3-(S)-(4-Ethyl-2-pyridin-3-yl-phenoxy)-butoxy]-2-methyl-phenyl}-propionic acid

The compound of 3-{4-[3-(S)-(4-ethyl-2-pyridin-3-yl-phenoxy)-butoxy]-2-methyl-phenyl}-propionic acid methyl ester (45 mg, 0.10 mmol, 62%) is prepared according to the procedure described in Example 31, Step B by using cesium carbonate (75 mg, 0.23 mmol), 4-ethyl-2-pyridin-3-yl-phenol (33 mg, 0.16 mmol) and 3-[4-(3-(S)-methanesulfonyloxy-butoxy)-phenyl]-propionic acid methyl ester (103 mg, 0.30 mmol) in DMF (0.7 mL). ES⁺ (m/e) 448.48 (M+H)⁺; R_{f}=0.15 hexanes: EtOAc (70:30). Work up of the above propionic acid methyl ester (45 mg, 0.10 mmol) in methanol (0.9 mL) as described in Example 26, Step E provides the title compound (62 mg, 0.09 mmol, 95%): ES⁺ (m/e) 434.40 (M+H)⁺.

### EXAMPLE 40

### 3-{4-[3-(S)-(4-Ethyl-2-pyridin-4-yl-phenoxy]-2-methyl-phenyl}-propionic acid

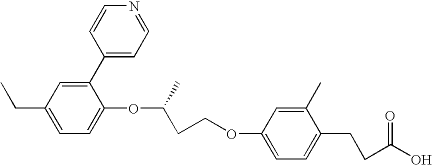

Cesium carbonate (68 mg, 0.21 mmol) is added to 4-ethyl-2-pyridin-4-yl-phenol (30 mg, 0.15 mmol) and 3-[4-(3-(S)-methanesulfonyloxy-butoxy)-phenyl]-propionic acid methyl ester (62 mg, 0.28 mmol) in DMF (0.5 mL), and the mixture is stirred under N₂ at 55° C. After 24 h, a 5 M aqueous solution of sodium hydroxide (1 mL) is added, and the mixture is cooled to ambient temperature for 5 h. The mixture is neutralized to pH=7 with a 1 M HCl and extracted with ethyl acetate. Organic layers are combined, washed with saturated aqueous sodium chloride, dried over magnesium sulfate, filtered and concentrated at reduced pressure. Oil was purified in HTC to obtain title compound (17 mg, 0.04 mmol, 26%): ES⁺ (m/e) 434.3 (M+H)⁺.

### EXAMPLE 41

### 3-{4-[3-(S)-(4-Chloro-2-pyridin-2-yl-phenoxy)-butoxy]-2-methyl-phenyl}-propionic acid

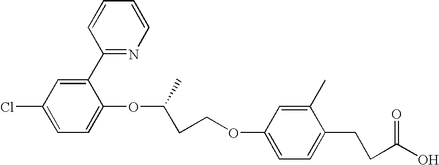

Cesium carbonate (720 mg, 2.21 mmol) is added to 4-chloro-2-pyridin-2-yl-phenol (350 mg, 1.70 mmol) and 3-[4-(3-(S)-methanesulfonyloxy-butoxy)-phenyl]-propionic acid methyl ester (702 mg, 2.04 mmol) in DMF (5.8 mL), and the mixture is stirred under N₂ at 55° C. After 24 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (85:15) gives 3-{4-[3-(S)-(4-Chloro-2-pyridin-2-yl-phenoxy)-butoxy]-2-methyl-phenyl}-propionic acid methyl ester (440 mg, 0.97 mmol, 57%): ES⁺ (m/e) 454.13 (M+H)⁺; R_{f}=0.35 hexanes:ethyl acetate (80:20).

A 5 M aqueous solution of sodium hydroxide (2.91 mL, 14 mmol) is added to the above propionic acid methyl ester (440 mg, 0.97 mmol) in methanol (7.0 mL), and the mixture is stirred at ambient temperature for 3 h. The mixture is neutralized to pH=7 with a 1 M HCl and extract with ethyl acetate. Organic layers are combined, washed with saturated aqueous sodium chloride, dried over magnesium sulfate, filtered and concentrated at reduced pressure to obtain title compound (62 mg, 0.09 mmol, 95%): ES⁺ (m/e) 434.40 (M+H)⁺.

### EXAMPLE 42

### 3-{4-[3-(2-Benzoyl-4-methoxy-phenoxy)-butoxy]-2-methyl-phenyl}-propionic acid

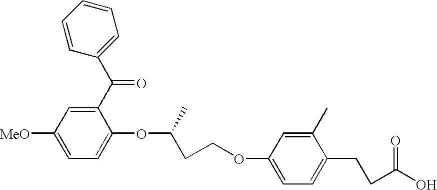

Cesium carbonate (58 mg, 0.18 mmol) is added to (2-hydroxy-5-methoxy-phenyl)-phenyl-methanone (30 mg, 0.13 mmol) and 3-[4-(3-(S)-methanesulfonyloxy-butoxy)-phenyl]-propionic acid methyl ester (54 mg, 0.16 mmol) in DMF (11.0 mL), and the mixture is stirred under N₂ at 55° C. After 24 h, a 5 M aqueous solution of sodium hydroxide (1 mL) is added and the mixture is cooled to ambient temperature in 5 h. The mixture is acidified to pH=2 with HCl (1M) and extracted with EtOAc. Organic layers are combined and washed with saturated aqueous sodium chloride, and then dried over magnesium sulfate, filtrated and concentrated at reduced pressure. Oil is purified in HTC to obtain the title compound: ES⁺ (m/e) 463.3 (M+H)⁺.

### EXAMPLE 43

### 3-{4-[3-(S)-(2-Benzoyl-4-fluoro-phyenoxy)-butoxy]-2-methyl-phenyl}-propionic acid

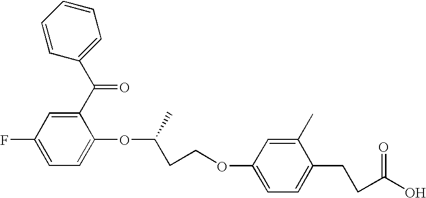

The title compound is prepared according to the procedure described in Example 42 using cesium carbonate (63 mg, 0.19 mmol), (5-fluoro-2-hydroxy-phenyl)-phenyl)-phenyl-methanone (30 mg, 0.16 mmol) and 3-[4-(3-(S)-methanesulfonyloxy-butoxy)-phenyl]-propionic acid methyl ester (57 mg, 0.16 mmol). ES⁺ (m/e) 451.3 (M+H)⁺.

### EXAMPLE 44

### 3-{4-[3-(S)-(4-Isopropyl-2-phenoxy-phenoxy)-butoxy]-2-methyl-phenyl}-propionic acid

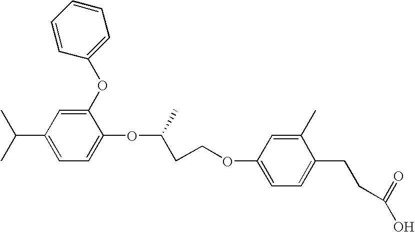

### Step A

### 4-Isopropyl-2-phenoxy-phenol

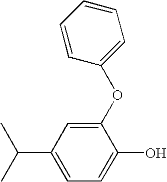

Cesium carbonate (4.3 g, 13.3 mmol) is added to phenol (1.05 g, 13.3 mmol) and 2-bromo-4-isopropyl-1-methoxy-benzene (1 g, 4.3 mmol) in 1-methyl-2-pyrrolidinone (15 mL). After 5 min., cupper chloride (1) (0.33 g, 3.3 mmol) and 2,2,6,6-tetramethyl-heptane-3,5-dione (0.30 g, 1.7 mmol) are added and the mixture is stirred at 120° C. under N₂. After 24 h, the mixture is cooled to ambient temperature and filtered and the solids are washed with EtOAc. Organic phase is washed with water and saturated aqueous sodium chloride, and then dried over magnesium sulfate, filtered and concentrated under reduced pressure. Purification by flash chromatography, silica, hexanes: EtOAc (95:5) provides 4-isopropyl-1-methoxy-2-phenoxy-benzene (1.0 g, 4.0 mmol, 94%): ES⁺ (m/e) 243.09 (M+H)⁺. A 4 M solution of boron tribromide (2.0 mL, 8.0 mmol) is added to 4-isopropyl-1-methoxy-2-phenoxy-benzene (1.0 g, 4.0 mmol) in DCM (4 mL) at −78° C. under N₂. The mixture is warmed to −5° C., and after 2 h, the mixture is cooled to 0° C. and diluted with water. Aqueous phase is extracted with additional DCM. Organic phase is washed with saturated aqueous sodium chloride, dried over magnesium sulfate, and concentrated. Purification by silica flash chromatography hexanes:EtOAc (95:5) provided the title compound (0.7 g, 3.3 mmol, 82%): ES⁺ (m/e) 227.02 (M−H)⁻, R_{f}=0.53 hexanes: EtOAc (90:10).

### Step B

### 3-{4-[3-(S)-(4-Isopropyl-2-phenoxy-phenoxy)-butoxy]-2-methyl-phenyl}-propionic acid

The compound of 3-{4-[3-(S)-(4-isopropyl-2-phenoxy-phenoxy)-butoxy]-2-methyl-phenyl}-propionic acid methyl ester (67 mg, 0.14 mmol, 73%) is prepared according to the procedure described in Example 31, Step B by using cesium carbonate (130 mg, 0.40 mmol), 4-isopropyl-2-phenoxy-phenol (44 mg, 0.19 mmol) and 3-[4-(3-(S)-methanesulfonyloxy-butoxy)-phenyl]-propionic acid methyl ester (86 mg, 0.25 mmol) in DMF (0.7 mL). ES⁺ (m/e) 499.36 (M+Na)⁺; R_{f}=0.51 hexanes: EtOAc (80:20). Work up of the above propionic acid methyl ester (67 mg, 0.14 mmol) in methanol (1.1 mL) as described in Example 20, Step D provides the title compound (63 mg, 0.13 mmol, 95%): ES⁺ (m/e) 463.31 (M+H)⁺.

### EXAMPLE 45

### {4-[3-(S)-(4-Isopropyl-2-phenoxy-phenoxy)-butoxy]-2-methyl-phenylsulfanyl}-acetic acid

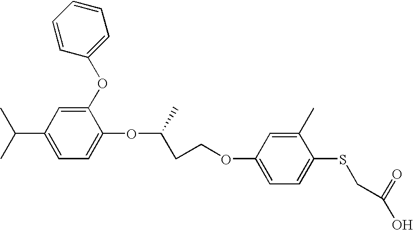

The compound of {4-[3-(S)-(4-isopropyl-2-phenoxy-phenoxy)-butoxy]-2-methyl-phenylsulfanyl}-acetic acid ethyl ester (0.39 g, 0.77 mmol, 70%) is prepared according to the procedure described in Example 31, Step B by using cesium carbonate (0.57 g, 1.74 mmol), 4-isopropyl-2-phenoxy-phenol (0.25 g, 1.09 mmol) and [4-(3-(S)-methanesulfonyloxy-butoxy)-2-methyl-phenylsulfanyl]acetic acid ethyl ester (0.53 g, 1.40 mmol) in DMF (7.0 mL). ES⁺ (m/e) 531.30 (M+Na)⁺; R_{f}=0.27 hexanes: EtOAc (80:20). Work up of the above acetic acid ethyl ester (0.39 g, 0.77 mmol) in ethanol (6.0 mL) as described in Example 20, Step D provides the title compound as a colorless oil (0.37 g, 0.77 mmol, 99%): ES⁺ (m/e) 503.29 (M+Na)⁺.

### EXAMPLE 46

### 3-{4-[3-(S)-(5-Chloro-3-phenoxy-pyridin-2-yloxy)-butoxy]-2-methyl-phenyl}-propionic acid

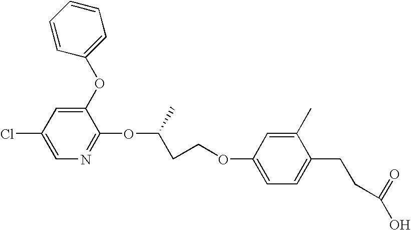

### Step A

### 5-Chloro-3-phenoxy-pyridin-2-ol

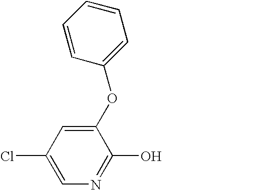

Cesium carbonate (8.1 g, 25 mmol) is added to phenol (2.35 g, 25 mmol) and 3-bromo-5-chloro-2-methoxy-pyridine (2.8 g, 12 mmol) in 1-methyl-2-pyrrolidinone (27 mL). After 5 min, cupper chloride (I) (0.62 g, 6.2 mmol) and 2,2,6,6-tetramethyl-heptane-3,5-dione (0.58 g, 3.1 mmol) are added and the mixture is stirred at 120° C. under N₂. After 24 h, the mixture is cooled to ambient temperature and filtered, and the solids are washed with EtOAc. Organic solution is washed with water and saturated aqueous sodium chloride, and then dried over magnesium sulfate, filtered and concentrated under reduced pressure. Purification by flash chromatography, silica, hexanes: EtOAc (95:5) provides 5-chloro-2-methoxy-3-phenoxy-pyridine (3 g, 12 mmol, 99%): ES⁺ (m/e) 235.98 (M+H)⁺; R_{f}=0.45 hexanes: EtOAc (90:10). HBr 48% (8 mL) is added to 5-chloro-2-methoxy-3-phenoxy-pyridine (3 g, 12 mmol) in acetic acid (20 mL), and the mixture is stirred at 105° C. for 10 min. The mixture is cooled to room temperature and neutralized to pH=7 with a 5 M aqueous solution of sodium hydroxide, and then extracted with EtOAc. Organic layers are combined, washed with saturated aqueous sodium chloride, dried over magnesium sulfate, filtered and concentrated under reduced pressure to obtain title compound as a colorless oil (0.37 g, 0.77 mmol, 99%): ES⁺ (m/e) 222.07 (M+H)⁺; ¹H NMR (400 MHz, CDCl₃) 7.39-7.43 (m, 2H), 7.22-7.26 (m, 2H), 7.09-7.10 (d, 2H, J=8 Hz), 6.80 (s, 1H).

### Step B

### 3-{4-[3-(S)-(5-Chloro-3-phenoxy-pyridin-2-yloxy)-butoxy]-2-methyl-phenyl}-propionic acid

Potassium carbonate (131 mg, 0.94 mmol) is added to 5-chloro-3-phenoxy-pyridin-2-ol (150 mg, 0.68 mmol) and 3-[4-(3-(S)-methanesulfonyloxy-butoxy)-phenyl]-propionic acid methyl ester (279 mg, 0.81 mmol) in DMF (2.5 mL), and the mixture is stirred under N₂ at 55° C. After 20 h, the mixture is cooled to ambient temperature and filtered and washed solid with EtOAc. The filtrate is washed with water and saturated aqueous sodium chloride, and then dried over magnesium sulfate, filtered and concentrated under reduce pressure. Purification by flash chromatography, silica, hexanes: EtOAc (95:5) gives 3-{4-[3-(S)-(5-chloro-3-phenoxy-pyridin-2-yloxy)-butoxy]-2-methyl-phenyl}-propionic acid methyl ester (100 mg, 0.21 mmol, 31%): ES⁺ (m/e) 470.27 (M+H)⁺; R_{f}=0.48 hexanes: EtOAc (80:20). Work up of the above propionic acid methyl ester (100 mg, 0.21 mmol) in methanol (1.5 mL) as described in Example 26, Step D provides the title compound (98 mg, 0.21 mmol, 95%): ES⁺ (m/e) 456.28 (M+H)⁺.

### EXAMPLE 47

### {4-[3-(S)-(5-Chloro-3-phenoxy-pyridin-2-yloxy)-butoxy]-2-methyl-phenylsulfanyl}-acetic acid

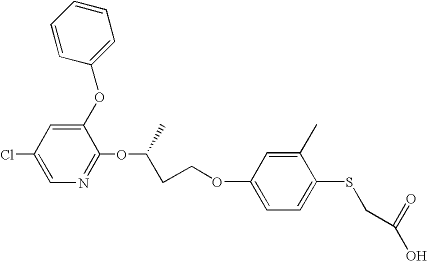

The compound of {4-[3-(S)-(5-chloro-3-phenoxy-pyridin-2-yloxy)-butoxy]-2-methyl-phenylsulfanyl}-acetic acid ethyl ester (0.10 g, 0.19 mmol, 32%) is prepared according to the procedure described in Example 46, Step B by using cesium carbonate (0.31 g, 0.96 mmol), 5-chloro-3-phenoxy-pyridin-2-ol (0.13 g, 0.60 mmol) and [4-(3-(S)-methanesulfonyloxy-butoxy)-2-methyl-phenylsulfanyl]acetic acid ethyl ester (0.29 g, 0.78 mmol) in DMF (3.0 mL). ES⁺ (m/e) 502.32 (M+H)⁺; R_{f}=0.51 hexanes:EtOAc (80:20).

Add a 5 M aqueous solution of sodium hydroxide (0.57 mL, 0.29 mmol) to the above acetic acid ethyl ester (0.10 g, 0.19 mmol) in ethanol (1.2 mL), and the mixture is stirred at ambient temperature for 8 h. The mixture is neutralized to pH=7 with a 1M HCl and then extracted with EtOAc. Organic layers are combined and washed with saturated aqueous sodium chloride, and then dried over magnesium sulfate, filtered and concentrated under reduced pressure to obtain title compound as a colorless oil (0.09 g, 0.18 mmol, 95%): ES⁺ (m/e) 474.20 (M+H)⁺.

### EXAMPLE 48

### 3-{4-[3-(S)-(5-Chloro-3-phenoxy-pyridin-2-yloxy)-butoxy]-2-ethyl-phenyl}-propionic acid

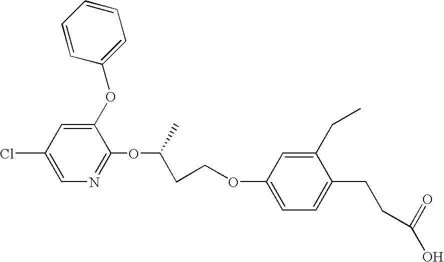

The compound of 3-{4-[3-(S)-(5-Chloro-3-phenoxy-pyridin-2-yloxy)-butoxy]-2-ethyl-phenyl}-propionic acid ethyl ester (0.18 g, 0.35 mmol, 52%) is prepared according to the procedure described in Example 46, Step B by using cesium carbonate (0.29 g, 0.88 mmol), 5-chloro-3-phenoxy-pyridin-2-ol (0.15 g, 0.67 mmol) and 3-[2-ethyl-4-3-(S)-methanesulfonyloxy-butoxy)-phenyl]-propionic acid ethyl ester (0.30 g, 0.81 mmol) in DMF (2.6 mL). ES⁺ (m/e) 520.11 (M+Na)⁺; R_{f}=0.56 hexanes: EtOAc (80:20). Work up of the above propionic acid ethyl ester (0.17 g, 0.35 mmol) in ethanol (2.5 mL) as described in Example 47 provides the title compound as a colorless oil (0.16 g, 0.34 mmol, 95%): ES⁺ (m/e) 492.06 (M+Na)⁺.

### EXAMPLE 49

### 3-{4-[3-(S)-(3-Benzoyl-5-chloro-pyridin-2-yloxy)-butoxy]-2-methyl-phenyl}-propionic acid

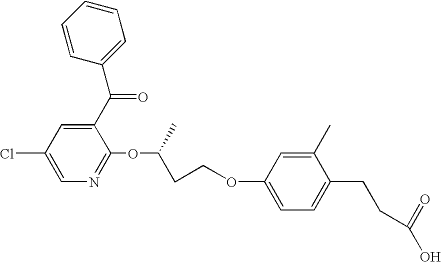

### Step A

### (5-Chloro-2-hydroxy-pyridin-3-yl)-phenyl-methanone

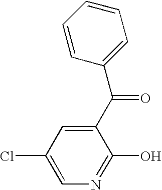

A 1.4 M solution of sec-BuLi in cyclohexane (1.1 mL, 1.5 mmol) is added dropwise for 20 min to 5-chloro-2-methoxy-pyridine(200 mg, 1.4 mmol) in THF (2.5 mL) at −78° C. under N₂. After stirring for 45 min., N-methoxy-N-methyl-benzamide (0.29 mL, 1.9 mmol) is added dropwise. After 1 h, a 1M solution of aqueous HCl (1 mL) is added, and the mixture is warmed to room temperature. The mixture is diluted with water and extracted with EtOAc. Organic phases are combined and washed with saturated aqueous sodium chloride, and then dried over magnesium sulfate, filtered and concentrated under reduced pressure to obtain an oil. Purification by flash chromatography, silica, hexanes: EtOAc (96:4) provides (5-chloro-2-methoxy-pyridin-3-yl)-phenyl-methanone: ES⁺ (m/e) 247.92 (M+H)⁺, R_{f}=0.35 hexanes: EtOAc (90:10). A 5.1 M solution of boron tribromide in DCM (0.15 mL, 0.81 mmol) is added dropwise to (5-chloro-2-methoxy-pyridin-3-yl)-phenyl-methanone (0.10 g, 0.40 mmol) in DCM (3 mL) at −78° C., and the mixture is stirred. The mixture is warmed slowly to ambient temperature, and after 8 h, the mixture is cooled to 0° C. and water is added carefully. The mixture is extracted with EtOAc. Organic phases are combined and washed with saturated aqueous sodium chloride, and then dried over magnesium sulfate and concentrated under reduced pressure to obtain title compound as a yellow solid (0.09 g, 0.38 mmol, 98%). ES⁺ (m/e) 232 (M)⁺.

### Step B

### 3-{4-[3-(S)-(3-Benzoyl-5-chloro-pyridin-2-yloxy)-butoxy]-2-methyl-phenyl}-propionic acid

The compound of 3-{4-[3-(S)-(3-Benzoyl-5-chloro-pyridin-2-yloxy)-butoxy]-2-methyl-phenyl}-propionic acid methyl ester (48 mg, 0.10 mmol, 50%) is prepared according to the procedure described in Example 46, Step B by using cesium carbonate (104 mg, 0.32 mmol), (5-chloro-2-hydroxy-pyridin-3-yl)-phenyl-methanone (47 mg, 0.20 mmol) and 3-[4-(3-(S)-methanesulfonyloxy-butoxy)-phenyl]-propionic acid methyl ester (89 mg, 0.26 mmol) in DMF (1.5 mL). ES⁺ (m/e) 504.17 (M+Na)⁺; R_{f}=0.46 hexanes: EtOAc (80:20). Work up of the above propionic acid methyl ester (47 mg, 0.10 mmol) in methanol (1.0 mL) as described in Example 47 provides the title compound (45 mg, 0.09 mmol, 95%): ES⁺ (m/e) 468.24 (M+H)⁺.

### EXAMPLE 50

### {4-[3-(S)-(3-Benzoyl-4-chloro-pyridin-2-yloxy)-butoxy]-2-methyl-phenylsulfanyl}-acetic acid

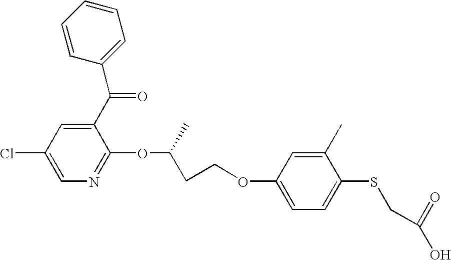

The compound of {4-[3-(S)-(3-benzoyl-4-chloro-pyridin-2-yloxy)-butoxy]-2-methyl-phenylsulfanyl}-acetic acid ethyl ester (43 mg, 0.08 mmol, 39%) is prepared according to the procedure described in Example 46, Step B by using cesium carbonate (0.11 g, 0.34 mmol), (5-chloro-2-hydroxy-pyridin-3-yl)-phenyl-methanone (0.05 g, 0.21 mmol) and [4-(3-(S)-methanesulfonyloxy-butoxy)-2-methyl-phenylsulfanyl]acetic acid ethyl ester (0.10 g, 0.27 mmol) in DMF (1.4 mL). ES⁺ (m/e) 514.17 (M+H)⁺; R_{f}=0.39 hexanes: EtOAc (80:20). Work up of the above acetic acid ethyl ester (43 mg, 0.08 mmol) in ethanol (1.0 mL) as described in Example 47 provides the title compound as a colorless oil (40 mg, 0.07 mmol, 95%). ES⁺ (m/e) 486.20 (M+H)⁺.

### EXAMPLE 51

### 3-{4-[3-(S)-(3-Benzoyl-5-trifluoromethyl-pyridin-2-yloxy)-butoxy]-2-methyl-phenyl}-propionic acid

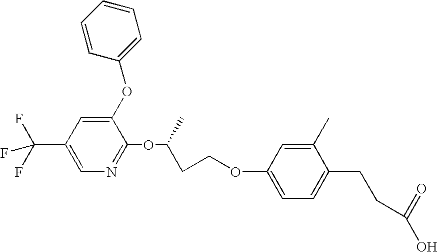

Cesium carbonate (114 mg, 0.35 mmol) is added to (2-hydroxy-5-trifluoromethyl-pyridin-3-yl)-phenyl-methanone (67 mg, 0.25 mmol) and 3-[4-(3-(S)-methanesulfonyloxy-butoxy)-phenyl]-propionic acid methyl ester (105 mg, 0.30 mmol) in DMF (1.2 mL), and the mixture is stirred under N₂ at 55° C. After 24 h, the mixture is cooled to ambient temperature, filtered and washed solid with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (95:5) gives 3-{4-[3-(S)-(3-benzoyl-5-trifluoromethyl-pyridin-2-yloxy)-butoxy]-2-methyl-phenyl}-propionic acid methyl ester (37 mg, 0.07 mmol, 30%): ES⁺ (m/e) 516.29 (M+H)⁺; R_{f}=0.21 hexanes:ethyl acetate (90:10).

A 5 M aqueous solution of sodium hydroxide (0.22 mL, 1.1 mmol) is added to the above propionic acid methyl ester (37 mg, 0.07 mmol) in methanol (0.6 mL), and the mixture is stirred at ambient temperature for 4 h. The mixture is neutralized to pH=7 with a 1 M HCl and extracted with ethyl acetate. Organic layers are combined, washed with saturated aqueous sodium chloride, dried over magnesium sulfate, filtered and concentrated at reduced pressure to obtain title compound (35 mg, 0.06 mmol, 95%): ES⁺ (m/e) 502.13 (M+H)⁺.

### EXAMPLE 52

### {4-[3-(S)-(3-Benzoyl-5-trifluoromethyl-pyridin-2-yloxy)-butoxy]-2-methyl-phenylsulfanyl}-acetic acid

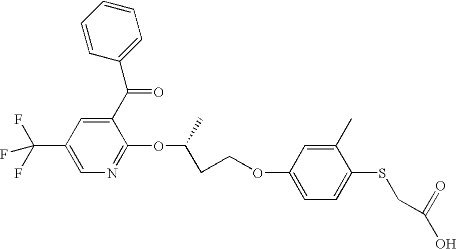

The compound of {4-[3-(S)-(3-benzoyl-5-trifluoromethyl-pyridin-2-yloxy)-butoxy]-2-methyl-phenylsulfanyl}-acetic acid ethyl ester (10 mg, 0.02 mmol, 12%) is prepared according to the procedure described in Example 46, Step B by using cesium carbonate (72 mg, 0.22 mmol), (2-hydroxy-5-trifluoromethyl-pyridin-3-yl)-phenyl-methanone (40 mg, 0.15 mmol) and [4-(3-(S)-methanesulfonyloxy-butoxy)-2-methyl-phenylsulfanyl]acetic acid ethyl ester (67 mg, 0.18 mmol) in DMF (0.7 mL). ES⁺ (m/e) 548.25 (M+H)⁺; R_{f}=0.36 hexanes: EtOAc (90:10). Work up of the above acetic acid ethyl ester (10 mg, 0.02 mmol) in ethanol (0.5 mL) as described in Example 47 provides the title compound as a colorless oil (9 mg, 0.02 mmol, 95%). ES⁺ (m/e) 520.08 (M+Na)⁺.

### EXAMPLE 53

### 3-{2-Methyl-4-[3-(S)-(3-phenoxy-5-trifluoromethyl-pyridin-2-yloxy)-butoxy]-phenyl}-propionic acid

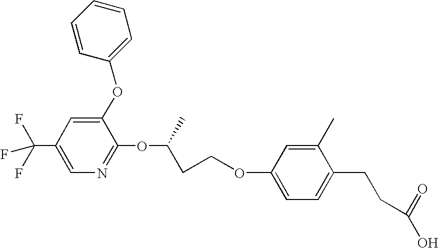

Cesium carbonate (83 mg, 0.25 mmol) is added to 3-phenoxy-5-trifluoromethyl-pyridin-2-ol (50 mg, 0.20 mmol) and 3-[4-(3-(S)-methanesulfonyloxy-butoxy)-phenyl]-propionic acid methyl ester (87 mg, 0.25 mmol) in DMF (0.9 mL), and the mixture is stirred under N₂ at 55° C. After 18 h, the mixture is cooled to ambient temperature, filtered and washed solid with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (93:7) gives 3-{2-methyl-4-[3-(S)-(3-phenoxy-5-trifluoromethyl-pyridin-2-yloxy)-butoxy]-phenyl}-propionic acid methyl ester (26 mg, 0.05 mmol, 27%): ES⁺ (m/e) 526.27 (M+Na)⁺; R_{f}=0.56 hexanes:ethyl acetate (80:20).

A 5 M aqueous solution of sodium hydroxide (0.30 mL, 1.5 mmol) is added to the above propionic acid methyl ester (50 mg, 0.10 mmol) in methanol (0.8 mL), and the mixture is stirred at ambient temperature for 4 h. The mixture is neutralized to pH=7 with a 1 M HCl and extracted with ethyl acetate. Organic layers are combined, washed with saturated aqueous sodium chloride, dried over magnesium sulfate, filtered and concentrated at reduced pressure to obtain title compound (47 mg, 0.08 mmol, 95%): ES⁺ (m/e) 512.23 (M+Na)⁺.

### EXAMPLE 54

### {2-Methyl-4-[3-(S)-(3-phenoxy-5-trifluoromethyl-pyridin-2-yloxy)-butoxy]-phenylsulfanyl}-acetic acid

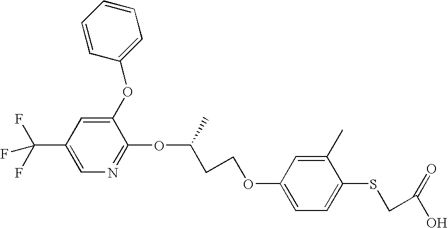

The compound of {2-Methyl-4-[3-(S)-(3-phenoxy-5-trifluoromethyl-pyridin-2-yloxy)-butoxy]-phenylsulfanyl}-acetic acid ethyl ester (33 mg, 0.06 mmol, 30%) is prepared according to the procedure described in Example 46, Step B by using cesium carbonate (81 mg, 0.25 mmol), 3-phenoxy-5-trifluoromethyl-pyridin-2-ol (53 mg, 0.21 mmol) and [4-(3-(S)-methanesulfonyloxy-butoxy)-2-methyl-phenylsulfanyl]acetic acid ethyl ester (93 mg, 0.25 mmol) in DMF (1.0 mL). ES⁺ (m/e) 558.22 (M+Na)⁺; R_{f}=0.61 hexanes: EtOAc (80:20). Work up of the above acetic acid ethyl ester (33 mg, 0.06 mmol) in ethanol (0.6 mL) as described in Example 47 provides the title compound as a colorless oil (30 mg, 0.06 mmol, 95%). ES⁺ (m/e) 530.26 (M+Na)⁺.

### EXAMPLE 55

### 3-{2-Ethyl-4-[3-(S)-(3-phenoxy-5-trifluoromethyl-pyridin-2-yloxy)-butoxy]-phenyl}-propionic acid

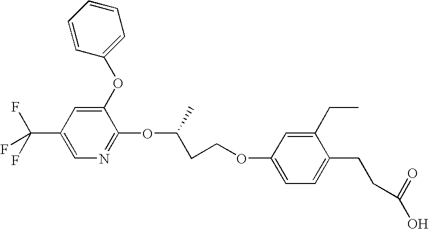

The compound of 3-{2-ethyl-4-[3-(S)-(3-phenoxy-5-trifluoromethyl-pyridin-2-yloxy)-butoxy]-phenyl}-propionic acid ethyl ester (0.07 g, 0.14 mmol, 22%) is prepared according to the procedure described in Example 46, Step B by using potassium carbonate (0.11 g, 0.81 mmol), 3-phenoxy-5-trifluoromethyl-pyridin-2-ol (0.16 g, 0.63 mmol) and 3-[2-ethyl-4-3-(S)-methanesulfonyloxy-butoxy)-phenyl]-propionic acid ethyl ester (0.27 g, 0.75 mmol) in DMF (4 mL). ES⁺ (m/e) 543.1 (M+Na)⁺; R_{f}=0.44 hexanes:EtOAc (80:20). Work up of the above propionic acid ethyl ester (0.07 g, 0.14 mmol) in ethanol (1.0 mL) as described in Example 47 provides the title compound as a colorless oil (0.06 g, 0.12 mmol, 95%). ES⁺ (m/e) 526.11 (M+Na)⁺.

### EXAMPLE 56

### 3-{2-Methyl-4-[3-(S)-(6-methyl-2-phenoxy-pyridin-3-yloxy)-butoxy]-phenyl}-propionic acid

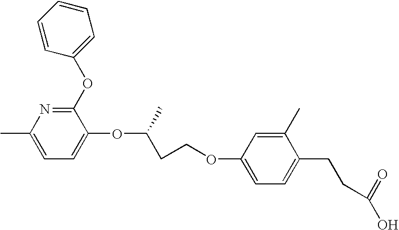

Cesium carbonate (227 mg, 0.49 mmol) is added to 6-methyl-2-phenoxy-pyridin-3-ol (100 mg, 0.20 mmol) and 3-[4-(3-(S)-methanesulfonyloxy-butoxy)-phenyl]-propionic acid methyl ester (205 mg, 0.59 mmol) in DMF (2.4 mL), and the mixture is stirred under N₂ at 55° C. After 24 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (84:16) gives 3-{2-methyl-4-[3-(S)-(6-methyl-2-phenoxy-pyridin-3-yloxy)-butoxy]-phenyl}-propionic acid methyl ester (11 mg, 0.24 mmol, 48%): ES⁺ (m/e) 450.40 (M+H)⁺; R_{f}=0.31 hexanes:ethyl acetate (80:20).

A 5 M aqueous solution of sodium hydroxide (0.72 mL, 3.6 mmol) is added to the above propionic acid methyl ester (100 mg, 0.240 mmol) in methanol (0.8 mL), and the mixture is stirred at ambient temperature for 4 h. The mixture is neutralized to pH=7 with a 1 M HCl and extracted with ethyl acetate. Organic layers are combined and washed with saturated aqueous sodium chloride, dried over magnesium sulfate, filtered and concentrate at reduced pressure to obtain title compound (47 mg, 0.08 mmol, 95%): ES⁺ (m/e) 436.48 (M+H)⁺.

### EXAMPLE 57

### 3-{4-[3-(S)-(3-Benzoyl-5-ethyl-pyridin-2-yloxy)-propoxy]-2-methyl-phenyl}-propionic acid

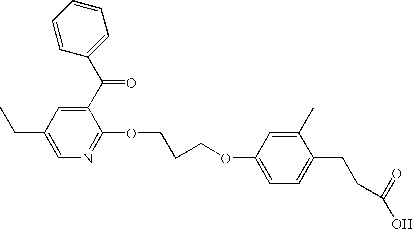

The compound of 3-{4-[3-(S)-(3-benzoyl-5-ethyl-pyridin-2-yloxy)-propoxy]-2-methyl-phenyl}-propionic acid methyl ester (40 mg, 0.09 mmol, 56%) is prepared according to the procedure described in Example 46, Step B by using cesium carbonate (80 mg, 0.25 mmol), (5-ethyl-2-hydroxy-pyridin-3-yl)-phenyl-methanone (35 mg, 0.15 mmol) and 3-[4-(3-methanesulfonyloxy-propoxy)-2-methyl-phenyl]-propionic acid methyl ester (66 mg, 0.20 mmol) in DMF (0.9 mL). ES⁺ (m/e) 462.15(M+H)⁺; R_{f}=0.27 hexanes: EtOAc (80:20). Work up of the above propionic acid methyl ester (40 mg, 0.09 mmol) in methanol (1.5 mL) as described in Example 47 provides the title compound (38 mg, 0.08 mmol, 95%). ES⁺ (m/e) 448.24 (M+H)⁺.

### EXAMPLE 58

### 3-{2-Methyl-4-[3-(S)-(3-phenoxy-5-trifluoromethyl-pyridin-2-yloxy)-propoxy]-phenyl}-propionic acid

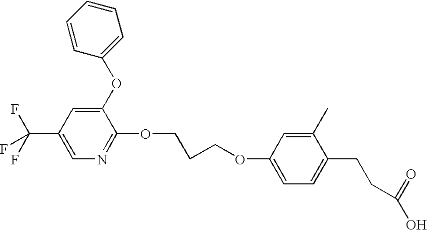

Cesium carbonate (46 mg, 0.14 mmol) is added to 3-phenoxy-5-trifluoromethyl-pyridin-2-ol (21 mg, 0.08 mmol) and 3-[4-(3-methanesulfonyloxy-propoxy)-2-methyl-phenyl]-propionic acid methyl ester methyl ester (33 mg, 0.10 mmol) in DMF (0.5 mL), and the mixture is stirred under N₂ at 55° C. After 24 h, a 5 M aqueous solution of sodium hydroxide (1 mL) is added and the mixture is cooled to ambient temperature for 5 h. The mixture is neutralized to pH=7 with a 1 M HCl and extracted with EtOAc. Organic layers are combined and washed with saturated aqueous sodium chloride and then dried over magnesium sulfate, filtrated and concentrated at reduced pressure. Oil is purified in HTC to obtain title compound as trifluoroacetate salt. ES⁺ (m/e) 476.1 (M+H)⁺.

### EXAMPLE 59

### 3-{4-[3-(S)-(5-Chloro-3-phenoxy-pyridin-2-yloxy)-propoxy]-2-methyl-phenyl}-propionic acid

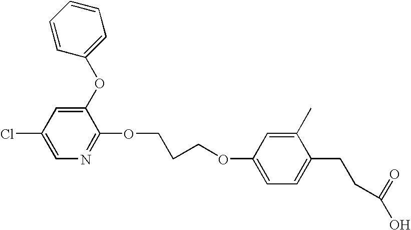

The title compound is prepared according to the procedure described in Example 58 by using cesium carbonate (34 mg, 0.14 mmol), 5-chloro-3-phenoxy-pyridin-2-ol (21 mg, 0.10 mmol) and 3-[4-(3-methanesulfonyloxy-propoxy)-2-methyl-phenyl]-propionic acid methyl ester methyl ester (30 mg, 0.09 mmol) in DMF (0.5 mL). ES⁺ (m/e) 442.0 (M+H)⁺.

### EXAMPLE 60

### 3-{2-Methyl-4-[3-(S)-(5-trifluoromethyl-[3,3′]bipyridinyl-2-yloxy)-butoxy]-phenyl}-propionic acid

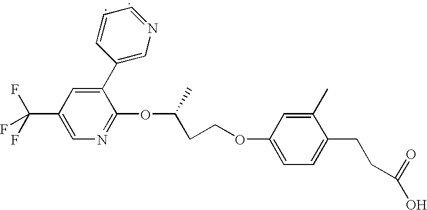

Cesium carbonate (38 mg, 0.11 mmol) is added to 5-trifluoromethyl-[3,3′]bipyridinyl-2-ol (22 mg, 0.09 mmol) and 3-[4-(3-(S)-methanesulfonyloxy-butoxy)-phenyl]-propionic acid methyl ester (38 mg, 0.11 mmol) in DMF(0.7 mL), and the mixture is stirred under N₂ at 55° C. After 18 h, the mixture is cooled to ambient temperature and filtered. The solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (70:30) gives 3-{2-methyl-4-[3-(S)-(5-trifluoromethyl-[3,3′]bipyridinyl-2-yloxy)-butoxy]-phenyl}-propionic methyl ester (24 mg, 0.05 mmol, 52%): ES⁺ (m/e) 489.1.5 (M+H)⁺; R_{f}=0.18 hexanes:ethyl acetate (70:30).

A 5M aqueous solution of sodium hydroxide (0.17 mL, 0.84 mmol) is added to the above propionic methyl ester (23 mg, 0.05 mmol) in methanol (0.5 mL) and the mixture is stirred at ambient temperature for 4 h. The mixture is neutralized to pH=7 with a 1 M HCl and extracted with ethyl acetate. Organic layers are combined and washed with saturated aqueous sodium chloride, and then dried over magnesium sulfate, filtered and concentrated at reduced pressure to obtain title compound (20 mg, 0.04 mmol, 95%) ES⁺ (m/e) 475.16 (M+H)⁺.

### EXAMPLE 61

### 3-{4-[3-(S)-(5-Chloro-[3,3′]bipyridinyl-2-yloxy)-butoxy]-2-methyl-phenyl}-propionic acid

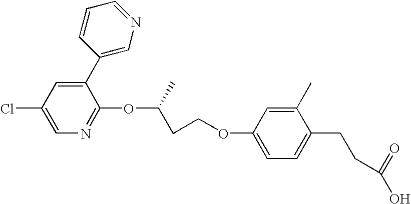

Cesium carbonate (67 mg, 0.21 mmol) is added to 5-chloro-[3,3′]bipyridinyl-2-ol (21 mg, 0.10 mmol) and 3-[4-(3-(S)-methanesulfonyloxy-butoxy)-phenyl]-propionic acid methyl ester (42 mg, 0.12 mmol) in DMF (0.7 mL), and the mixture is stirred under N₂ at 55° C. After 18 h, the mixture is cooled to ambient temperature and filtered. Solid is washed with ethyl acetate. The filtrate is washed with water and saturated aqueous sodium chloride, and then dried over magnesium sulfate, filtered and concentrated under reduce pressure. Purification by flash chromatography, silica, hexanes:ethyl acetate (60:40) gives 3-{4-[3-(S)-(5-chloro-[3,3′]bipyridinyl-2-yloxy)-butoxy]-2-methyl-phenyl}-propionic acid methyl ester (18 mg, 0.04 mmol, 40%): ES⁺ (m/e) 455.15 (M+H)⁺; R_{f}=0.32 hexanes:ethyl acetate (60:40).

A 5 M aqueous solution of sodium hydroxide (0.15 mL, 0.70 mmol) is added to the above propionic acid methyl ester (18 mg, 0.04 mmol) in methanol (0.6 mL), and the mixture is stirred at ambient temperature for 4 h. The mixture is neutralized to pH=7 with a 1 M HCl and extracted with ethyl acetate. Organic layers are combined and washed with saturated aqueous sodium chloride, and then dried over magnesium sulfate, filtered and concentrated at reduced pressure to obtain title compound (15 mg, 0.03 mmol, 90%) ES⁺ (m/e) 441.08 (M+H)⁺.

### EXAMPLE 62

### 3-{2-Ethyl-4-[3-(S)-(5-trifluoromethyl-[3,3′]bipyridinyl-2-yloxy)-butoxy]-phenyl}-propionic acid

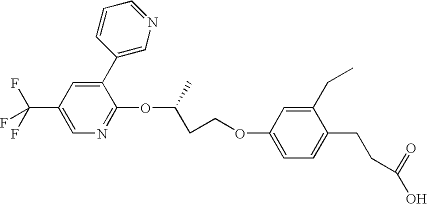

The compound of 3-{2-ethyl-4-[3-(S)-(5-trifluoromethyl-[3,3′]bipyridinyl-2-yloxy)-butoxy]-phenyl}-propionic acid ethyl ester (0.10 g, 0.19 mmol, 47%) is prepared according to the procedure described in Example 46, Step B by using cesium carbonate (0.19 g, 0.58 mmol), 5-trifluoromethyl-[3,3′]bipyridinyl-2-ol (0.10 g, 0.41 mmol) and 3-[2-ethyl-4-3-(S)-methanesulfonyloxy-butoxy)-phenyl]-propionic acid ethyl ester (0.18 g, 0.49 mmol) in DMF (3 mL). ES⁺ (m/e) 517.2 (M+H)⁺; R_{f}=0.33 hexanes:EtOAc (80:20). Work up of the above propionic acid ethyl ester (0.10 g, 0.19 mmol) in ethanol (1.0 mL) as described in Example 47 provides the title compound as a colorless oil (0.09 g, 0.17 mmol, 90%). ES⁺ (m/e) 489.13 (M+H)⁺.

### EXAMPLE 63

### (R)-3-{2-Chloro-4-[3-(4-chloro-2-phenoxy-phenoxy)-butoxy]-phenyl}-propionic acid

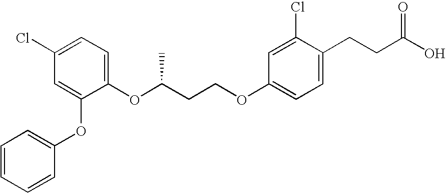

### Step A

### (S)-Acetic acid 3-hydroxy-butyl ester

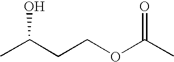

A mixture of (S)-(+)-1,3-butanediol (10.0 g, 0.1 mol) and 2,4,6-collidine (27 g, 0.2 mol) in DCM (100 mL) is cooled to −78° C. The reaction is then treated dropwise with acetyl chloride (10.4 g, 0.13 mol), and stirred for 2 hr at −78° C. The reaction is then allowed to warm to rt and stir for an additional hour. The reaction is then quenched with 1N HCl and extracted with DCM. The organic layer is separated, washed with brine, and dried over Na₂SO₄. The organic is filtered, and the solvent is removed to afford 9.77 g (66%) of acetic acid 3-hydroxy-butyl ester. ¹H NMR (400 MHz, CDCl₃); MS (ES⁺) m/z mass calcd for C₆H₁₂O₃ 132, found 133 (M+1).

### Step B

### (S)-Acetic acid 3-(toluene-4-sulfonyloxy)-butyl ester

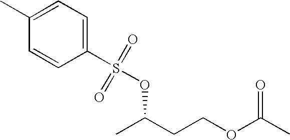

A solution of acetic acid 3-hydroxy-butyl ester (9.8 g, 70 mmol) in DCM (50 mL) is cooled to 0° C. The solution is treated with p-toluenesulfonyl chloride (16.9 g, 90 mmol), TEA (9 g, 90 mmol), and DMAP (2.3 g, 18.5 mmol). The mixture is stirred for 1 hr at 0° C., and then warmed to rt. The reaction is stirred overnight at rt. The reaction is then diluted in water and extracted with DCM. The organic layer is separated, washed with brine, and dried over sodium sulfate. The organic is filtered, and the solvent is removed to afford the crude product. The crude is purified by silica gel column chromatography using 9/1 hexanes/EtOAc to elute the pure product. The solvent is removed to afford 11.6 g (55%) of the desired product. ¹H NMR (400 MHz, CDCl₃); MS (ES⁺) m/z mass calcd for C₁₃H₁₈O₅S 286, found 287 (M+1, 100%).

### Step C

### (R)-3-(4-Chloro-2-phenoxy-phenoxy)-butan-1-ol

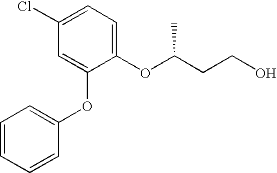

A solution of (R)-acetic acid 3-(toluene-4-sulfonyloxy)-butyl ester (5.89 g, 21 mmol) and 4-chloro-2-phenoxy-phenol (5.0 g, 23 mmol) in DMF (50 mL) is treated with cesium carbonate (7.4 g, 23 mmol). The solution is heated to 60° C. and stirred overnight. The reaction is cooled and quenched with 1N HCl. The solution is partitioned in EtOAc and water. The organic is separated, washed with brine, and dried over sodium sulfate. The organic is filtered, and the solvent is removed to afford acetic acid 3-(4-chloro-2-phenoxy-phenoxy)-butyl ester, which is then diluted in methanol (100 mL) and treated with potassium carbonate (5.68 g, 40 mmol). The reaction is stirred for 2 hours at rt. The reaction is then partitioned in EtOAc and water. The organic layer is separated, washed with brine, and dried over sodium sulfate. The organic is filtered and the solvent is removed to afford the crude product. The crude is purified by silica gel column chromatography using 1/1 hexanes/EtOAc to elute the pure product. The solvent is removed to afford 4.35 g (72%) of the desired product. ¹H NMR (400 MHz, CDCl₃); MS (ES⁺) m/z mass calcd for C₁₆H₁₇ClO₃ 292, found 293 (M+1, 100%).

### Step D

### (R)-Methanesulfonic acid 3-(4-chloro-2-phenoxy-phenoxy)-butyl ester

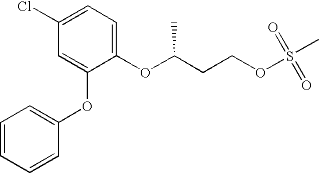

A solution of 3-(4-chloro-2-phenoxy-phenoxy)-butan-1-ol (4.35 g, 15 mmol) in DCM (50 mL) is cooled to 0° C. The solution is then treated with TEA (1.8 g, 18 mmol), MsCl (2.0 g, 18 mmol), and DMAP (0.454 g, 4 mmol). The reaction is stirred for 2 hours at 0° C. The reaction is then diluted in water and extracted with DCM. The organic is separated, washed with brine, and dried over sodium sulfate. The organic is filtered and the solvent is removed to afford 5.4 g (98%) of the desired product. ¹H NMR (400 MHz, CDCl₃); MS (ES⁺) m/z mass calcd for C₁₇H₁₉ClO₅S 370, found 371 (M+1, 100%).

### Step E

### (R)-3-{2-Chloro-4-[3-(4-chloro-2-phenoxy-phenoxy)-butoxy]-phenyl}-propionic acid

A solution of methanesulfonic acid 3-(4-chloro-2-phenoxy-phenoxy)-butyl ester (89 mg, 0.24 mmol) and 3-(2-chloro-4-hydroxy-phenyl)-propionic acid ethyl ester (50 mg, 0.22 mmol) in DMF (5 mL) is treated with cesium carbonate (85 mg, 0.26 mmol). The reaction is heated to 50° C. and stirred overnight. The reaction is treated with aqueous 5N NaOH (0.4 mL, 2.2 mmol) and stirred for 2 additional hours at 50° C. The reaction is cooled and quenched with 1N HCl to pH=4. The reaction is extracted with Et₂O. The organic is washed with brine, dried over sodium sulfate, filtered, and the solvent is removed. The crude product is purified by prep HPLC to afford 70 mg (67%) of the desired product. ¹H NMR (400 MHz, CDCl₃); MS (ES⁺) m/z mass calcd for C₂₅H₂₄Cl₂O₅ 370, found 371 (M+1, 100%).

### EXAMPLE 64

### (R)-3-{4-[3-(4-Chloro-2-phenoxy-phenoxy)-butoxy]-2-fluoro-phenyl}-propionic acid

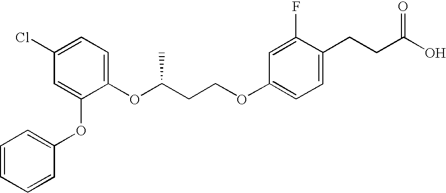

The procedure from Example 63, Step E is utilized with 3-(2-fluoro-4-hydroxy-phenyl)-propionic acid ethyl ester to afford 73 mg (66%) of desired product. ¹H NMR (400 MHz, CDCl₃); MS (ES) m/z mass calcd for C₂₅H₂₄ClFO₅ 458, found 459 (M+1, 100%).

### EXAMPLE 65

### (R)-3-{4-[3-(4-Chloro-2-phenoxy-phenoxy)-butoxy]-2-ethyl-phenyl}-propionic acid

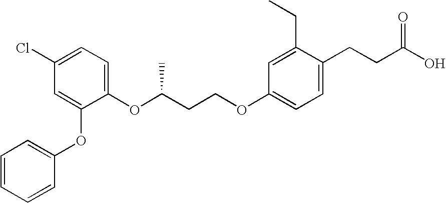

The procedure from Example 63, Step E is utilized with 3-(2-ethyl-4-hydroxy-phenyl)-propionic acid ethyl ester to afford 4 mg (4%) [symbol] desired product. ¹H NMR (400 MHz, CDCl₃); MS (ES⁺) m/z mass calcd for C₂₇ [symbol] ClO₅ 468, found 469 (M+1, 100%).

### EXAMPLE 66

### (R)-4-{4-[3-(4-Chloro-2-phenoxy-phenoxy)-butoxy]-2-methyl-phenyl}-butyric acid

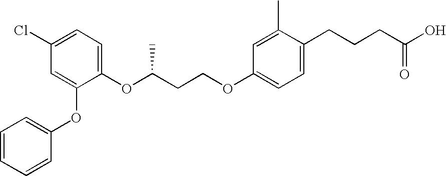

The procedure from Example 63, Step E is utilized with 4-(4-hydroxy-2-methyl-phenyl)-butyric acid ethyl ester to afford 54 mg (50%) of desired product. ¹H NMR (400 MHz, CDCl₃); MS (ES⁺) m/z mass calcd for C₂₇H₂₉ClO₅ 468, found 469 (M+1, 100%).

### EXAMPLE 67

### (R)-3-{4-[3-(4-Chloro-2-phenoxy-phenoxy)-butoxy]-phenyl}-propionic acid

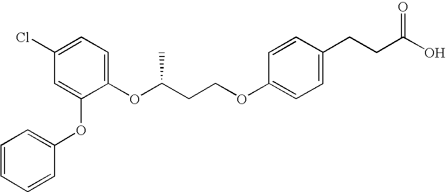

The procedure from Example 63, Step E is utilized with 3-(4-hydroxy-phenyl)-propionic acid ethyl ester to afford 53 mg (44%) of desired product. ¹H NMR (400 MHz, CDCl₃); MS (ES⁺) m/z mass calcd for C₂₅H₂₅ClO₅ 440, found 441 (M+1, 100%).

### EXAMPLE 68

### (R)-3-{4-[3-(2-Benzoyl-4-ethyl-phenoxy)-butoxy]-2-chloro-phenyl}-propionic acid

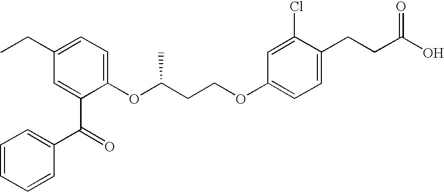

### Step A

### (R)-[5-Ethyl-2-(3-hydroxy-1-methyl-propoxy)-phenyl]-phenyl-methanone

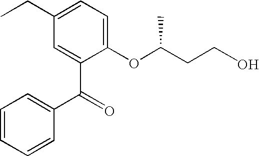

The procedure from Example 63, Step C is utilized with (5-ethyl-2-hydroxy-phenyl)-phenyl-methanone to afford 1.4 g (69%) of the desired product. ¹H NMR (400 MHz, CDCl₃); MS (ES⁺) m/z mass calcd for C₁₉H₂₂O₃ 298, found 299 (M+1, 100%).

### Step B

### (R)-Methanesulfonic acid 3-(2-benzoyl-4-ethyl-phenoxy)-butyl ester

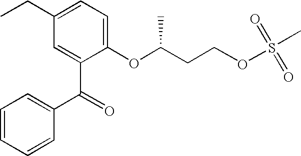

The procedure from Example 63, Step D is utilized with [5-ethyl-2-(3-hydroxy-1-methyl-propoxy)-phenyl]-phenyl-methanone (1.4 g, 5 mmol) to afford 1.7 g (98%) of the desired product. ¹H NMR (400 MHz, CDCl₃); MS (ES⁺) m/z mass calcd for C₂₀H₂₄O₅S 376, found 377 (M+1, 100%).

### Step C

### (R)-3-{4-[3-(2-Benzoyl-4-ethyl-phenoxy)-butoxy]-2-chloro-phenyl}-propionic acid

The procedure from Example 63, Step E is utilized with 3-(2-chloro-4-hydroxy-phenyl)-propionic acid ethyl ester to afford 61 mg (58%) of desired product. ¹H NMR (400 MHz, CDCl₃); MS (ES⁺) m/z mass calcd for C₂₈H₂₉ClO₅ 480, found 481 (M+1, 100%).

### EXAMPLE 69

### (R)-3-{4-[3-(2-Benzoyl-4-ethyl-phenoxy)-butoxy]-2-fluoro-phenyl}-propionic acid

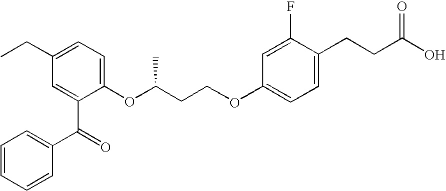

The procedure from Example 63, Step E is utilized with 3-(2-Fluoro-4-hydroxy-phenyl)-propionic acid ethyl ester to afford 61 mg (58%) of desired product. ¹H NMR (400 MHz, CDCl₃); MS (ES⁺) m/z mass calcd for C₂₈H₂₉FO₅ 464, found 465 (M+1, 100%).

### EXAMPLE 70

### (R)-3-{4-[3-(2-Benzoyl-4-ethyl-phenoxy)-butoxy]-phenyl}-propionic acid

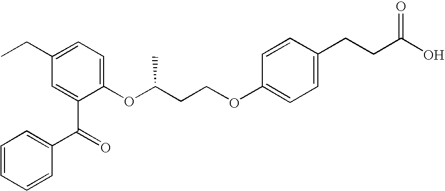

The procedure from Example 63, Step E is utilized with 3-(4-hydroxy-phenyl)-propionic acid ethyl ester to afford 59 mg (49%) of desired product. ¹H NMR (400 MHz, CDCl₃); MS (ES⁺) m/z mass calcd for C₂₈H₃₀O₅ 464, found 465 (M+1, 100%).

### EXAMPLE 71

### (R)-3-{4-[3-(4-Chloro-2-phenoxy-phenoxy)-butoxy]-phenyl}-3-methyl-butyric acid

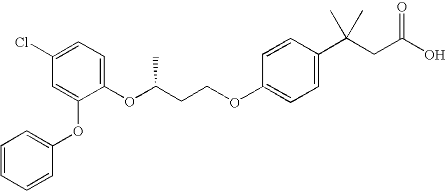

### Step A

### 3-(4-Hydroxy-phenyl)-3-methyl-butyric acid methyl ester

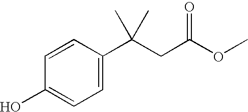

A solution of 3-(4-hydroxy-phenyl)-3-methyl-butyric acid (1.0 g, 5.15 mmol) in MeOH (25 mL) is treated with concentrated sulfuric acid (8 mL). The reaction is stirred overnight at rt. The reaction is cooled to 0° C. and quenched with 5.0N aqueous sodium hydroxide to pH=8. The aqueous layer is extracted with ethyl acetate. The organic layer is dried over sodium sulfate, filtered, and the solvent is removed to afford 780 mg (73%) of the title compound. ¹H NMR (400 MHz, CDCl₃); MS (ES⁺) m/z mass calcd for C₁₁H₁₄O₃ 194, found 195 (M+1, 100%).

### Step B

### (R)-3-{4-[3-(4-Chloro-2-phenoxy-phenoxy)-butoxy]-phenyl}-3-methyl-butyric acid methyl ester

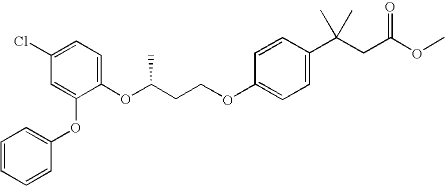

A solution of methanesulfonic acid 3-(4-chloro-2-phenoxy-phenoxy)-butyl ester (100 mg, 0.27 mmol) and 3-(4-hydroxy-phenyl)-3-methyl-butyric acid methyl ester (62 mg, 0.30 mmol) in DMF (10 mL) is treated with cesium carbonate (105 mg, 0.32 mmol). The reaction is heated to 50° C. and stirred overnight. The reaction is then cooled and quenched with 1N HCl to pH=7. The reaction is extracted with Et₂O. The organic is washed with brine, dried over sodium sulfate, filtered, and the solvent is removed. The crude is purified by silica gel column chromatography using 3/1 hexanes/EtOAc to elute the pure product. The solvent is removed to afford 80 mg (62%) of desired product. ¹H NMR (400 MHz, CDCl₃); MS (ES⁺) m/z mass calcd for C₂₈H₃₁ClO₅ 482, found 483 (M+1, 100%).

### Step C

### (R)-3-{4-[3-(4-Chloro-2-phenoxy-phenoxy)-butoxy]-phenyl}-3-methyl-butyric acid

A solution of 3-{4-[3-(4-chloro-2-phenoxy-phenoxy)-butoxy]-phenyl}-3-methyl-butyric acid methyl ester (80 mg, 0.17 mmol) in MeOH (15 mL) is treated with 5N aqueous sodium hydroxide (0.3 mL). The reaction is heated to reflux and stirred for 3 hours. The reaction is then cooled and adjusted to pH=4 with 1N aqueous hydrochloric acid. The solution is extracted with EtOAc. The organic is washed with brine, dried over sodium sulfate, filtered, and the solvent is removed to afford 61 mg (78%) of desired product. ¹H NMR (400 MHz, CDCl₃); MS (ES⁺) m/z mass calcd for C₂₇H₂₉ClO₅ 468, found 469 (M+1, 100%).

### EXAMPLE 72

### (R)-3-{4-[3-(4-Chloro-2-phenoxy-phenoxy)-butoxy]-2-propyl-phenyl}-propionic acid

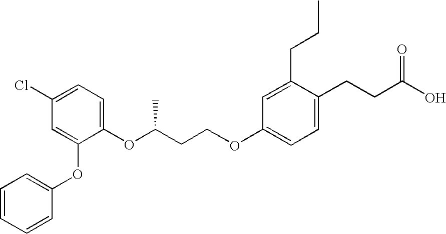

### Step A

### (R)-3-{4-[3-(4-Chloro-2-phenoxy-phenoxy)-butoxy]-2-propyl-phenyl}-propionic acid ethyl ester

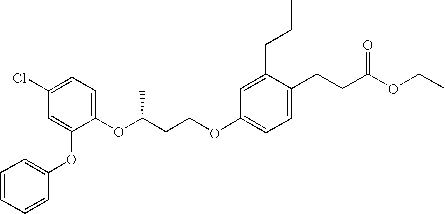

The procedure from Example 71, Step B is utilized with 3-(4-hydroxy-2-propyl-phenyl)-propionic acid ethyl ester (2159493) to afford 90 mg (78%) of desired product. ¹H NMR (400 MHz, CDCl₃); MS (ES⁺) m/z mass calcd for C₃₀H₃₅ClO₅ 510, found 511 (M+1, 100%).

### Step B

### (R)-3-{4-[3-(4-Chloro-2-phenoxy-phenoxy)-butoxy]-2-propyl-phenyl}-propionic acid

A solution of (R)-3-{4-[3-(4-chloro-2-phenoxy-phenoxy)-butoxy]-2-propyl-phenyl}-propionic acid ethyl ester (90 mg, 0.18 mmol) in EtOH (5 mL) is treated with 5.0N aqueous sodium hydroxide. The reaction is heated to 80° C. and stirred for 4 hours. The reaction is cooled to rt and quenched with 1.0N aqueous hydrochloric acid to pH=4. The aqueous is extracted with diethyl ether. The organic layer is washed with brine, and then dried over sodium sulfate and filtered. The solvent is removed to afford 81 mg (95%) of desired product. ¹H NMR (400 MHz, CDCl₃); MS (ES⁺) m/z mass calcd for C₂₈H₃₁ClO₅ 482, found 483 (M+1, 100%).

### EXAMPLE 73

### (R)-3-{4-[3-(4-Chloro-2-phenoxy-phenoxy)-butoxy]-2,6-dimethyl-phenyl}-propionic acid

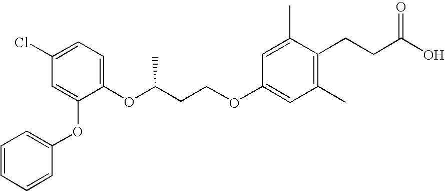

### Step A

### (R)-3-{4-[3-(4-Chloro-2-phenoxy-phenoxy)-butoxy]-2,6-dimethyl-phenyl}-propionic acid ethyl ester

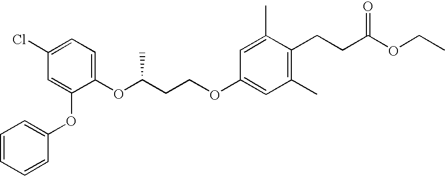

The procedure from Example 71, Step B is utilized with 3-(4-hydroxy-2,6-dimethyl-phenyl)-propionic acid ethyl ester (2190971) to afford 102 mg (91%) of desired product. ¹H NMR (400 MHz, CDCl₃); MS (ES⁺) m/z mass calcd for C₂₉H₃₃ClO₅ 496, found 497 (M+1, 100%).

### Step B

### (R)-3-{4-[3-(4-Chloro-2-phenoxy-phenoxy)-butoxy]-2,6-dimethyl-phenyl}-propionic acid

The procedure from Example 72, Step C is utilized with (R)-3-{4-[3-(4-chloro-2-phenoxy-phenoxy)-butoxy]-2,6-dimethyl-phenyl}-propionic acid ethyl ester to afford 82 mg (87%) of desired product. ¹H NMR (400 MHz, CDCl₃); MS (ES⁺) m/z mass calcd for C₂₇H₂₉ClO₅ 468, found 469 (M+1, 100%).

### EXAMPLE 74

### (R)-{4-[3-(4-Chloro-2-phenoxy-phenoxy)-butoxy]-2-ethyl-phenylsulfanyl}-acetic acid

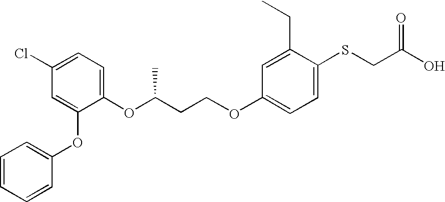

### Step A

### (R)-{4-[3-(4-Chloro-2-phenoxy-phenoxy)-butoxy]-2-ethyl-phenylsulfanyl}-acetic acid ethyl ester

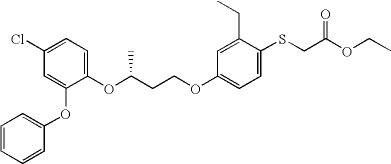

The procedure from Example 71, Step B is utilized with (2-ethyl-4-hydroxy-phenylsulfanyl)-acetic acid ethyl ester to afford 42 mg (39%) of desired product. ¹H NMR (400 MHz, CDCl₃); MS (ES⁺) m/z mass calcd for C₂₈H₃₁ClO₅S 514, found 515 (M+1, 100%).

### Step B

### (R)-{4-[3-(4-Chloro-2-phenoxy-phenoxy)-butoxy]-2-ethyl-phenylsulfanyl}-acetic acid

The procedure from Example 72, Step C is utilized with (R)-{4-[3-(4-chloro-2-phenoxy-phenoxy)-butoxy]-2-ethyl-phenylsulfanyl}-acetic acid ethyl ester to afford 25 mg (63%) of desired product. ¹H NMR (400 MHz, CDCl₃); MS (ES⁺) m/z mass calcd for C₂₆H₂₇ClO₅S 486, found 487 (M+1, 100%).

### EXAMPLE 75

### (R)-3-{4-[3-(4-Chloro-2-phenoxy-phenoxy)-pentyloxy]-2-methyl-phenyl}-propionic acid

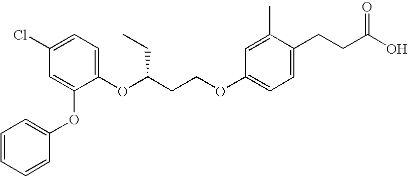

### Step A

### (R,S)-Toluene-4-sulfonic acid 3-hydroxy-pentyl esterluene-4-sulfonic acid 3-hydroxy-pentyl ester

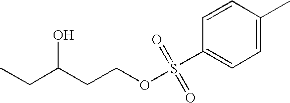

A solution of (R,S)-pentane-1,3-diol (20.0 g, 0.19 mol, 2148539) and TEA (23.3 g, 0.23 mol) in methylene chloride (400 mL) is treated with dibutyltin oxide (0.96 g, 3.8 mmol). The reaction is stirred at rt and treated portion wise with p-toluenesulfonyl chloride (36.6 g, 0.19 mol). The reaction is stirred overnight at rt. The reaction is diluted in water and neutralized to pH=7 with 1N aqueous hydrochloric acid. The aqueous is extracted with methylene chloride. The organic is dried over sodium sulfate, filtered, and the solvent removed to afford the crude product. The crude product is purified by silica gel column chromatography using 3/2 hexanes/EtOAc to elute the pure product. The solvent is removed to afford 34.3 g (69%) of desired product. ¹H NMR (400 MHz, CDCl₃); MS (ES⁺) m/z mass calcd for C₁₂H₁₈O₄S 258, found 259 (M+1).

### Step B

### (R,S)-3-[4-(3-Hydroxy-pentyloxy)-2-methyl-phenyl]-propionic acid methyl ester

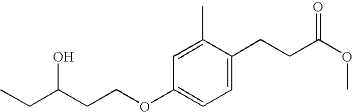

A solution of (R,S)-toluene-4-sulfonic acid 3-hydroxy-pentyl esterluene-4-sulfonic acid 3-hydroxy-pentyl ester (34.3 g, 0.13 mol) and 3-(4-hydroxy-2-methyl-phenyl)-propionic acid methyl ester (28.4 g, 0.15 mol) are combined in DMF (3.00 mL). The solution is treated with cesium carbonate (52 g, 0.16 mol) and heated to 55° C. The reaction is stirred overnight. The reaction is cooled and quenched with 1N HCl. The reaction is extracted with EtOAc. The organic is washed with brine, dried over sodium sulfate, filtered, and the solvent is removed. The crude is purified by silica gel column chromatography using 4/1 hexanes/EtOAc to elute the pure product. The solvent is removed to afford 5.4 g (15%) of the desired product. ¹H NMR (400 MHz, CDCl₃); MS (ES⁺) m/z mass calcd for C₁₆H₂₄O₄ 280, found 281 (M+1, 100%).

### Step C

### (S)-3-[4-(3-Hydroxy-pentyloxy)-2-methyl-phenyl]-propionic acid methyl ester

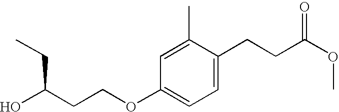

The compound of (R,S)-3-[4-(3-hydroxy-pentyloxy)-2-methyl-phenyl]-propionic acid methyl ester is purified by HPLC using a 4.6×250 mm Chiralpak AD column. The pure chiral compound is eluted using May 5, 1990 NPA/methanol/heptane. The solvent is removed to afford the desired product (95.6% ee). ¹H NMR (400 MHz, CDCl₃); MS (ES⁺) m/z mass calcd for C₁₆H₂₄O₄ 280, found 281 (M+1, 100%).

### Step D

### (S)-3-[4-(3-Methanesulfonyloxy-pentyloxy)-2-methyl-phenyl]-propionic acid methyl ester

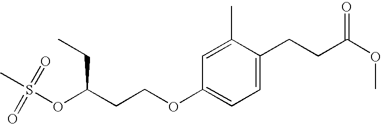

A solution of (S)-3-[4-(3-hydroxy-pentyloxy)-2-methyl-phenyl]-propionic acid methyl ester (0.2 g, 0.7 mmol) and TEA (0.108 g, 1.07 mmol) are combined in methylene chloride (10 mL) and cooled to 0° C. The solution is then treated with MsCl (0.098 g, 0.86 mmol) and stirred for 2 hours at 0° C. The reaction is then quenched with water and extracted with methylene chloride. The organic is dried over sodium sulfate, filtered, and the solvent removed to afford 0.25 g (quantitative) of the desired product. ¹H NMR (400 MHz, CDCl₃); MS (ES⁺) m/z mass calcd for C₁₇H₂₆O₆S 358, found 359 (M+1, 100%).

### Step E

### (R)-3-{4-[3-(4-Chloro-2-phenoxy-phenoxy)-pentyloxy]-2-methyl-phenyl}-propionic acid

A solution of (S)-3-[4-(3-methanesulfonyloxy-pentyloxy)-2-methyl-phenyl]-propionic acid methyl ester (125 mg, 0.35 mmol) and 4-Chloro-2-phenoxy-phenol (70 mg, 0.32 mmol) in DMF (5 mL) is treated with cesium carbonate (124 mg, 0.38 mmol). The reaction is heated to 60° C. and stirred overnight. The reaction is then treated with aqueous 5N NaOH (0.4 mL, 2.2 mmol) and stirred for 2 additional hours at 50° C. The reaction is then cooled and quenched with 1N HCl to pH=4. The reaction is then extracted with Et₂O. The organic is washed with brine, dried over sodium sulfate, filtered, and the solvent removed. The crude product is purified by prep HPLC to afford 63 mg (42%) of the desired product. ¹H NMR (400 MHz, CDCl₃); MS (ES⁺) m/z mass calcd for C₂₇H₂₉ClO₅ 468, found 469 (M+1, 100%).

### EXAMPLE 76

### (R)-3-{4-[3-(2-Benzoyl-4-ethyl-phenoxy)-pentyloxy]-2-methyl-phenyl}-propionic acid

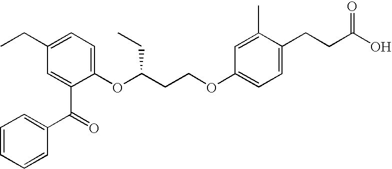

The procedure from Example 75, Step E is utilized with (5-ethyl-2-hydroxy-phenyl)-phenyl-methanone to afford 77 mg (50%) of the desired product. ¹H NMR (400 MHz, CDCl₃); MS (ES⁺) m/z mass calcd for C₃₀H₃₄O₅ 474, found 475 (M+1, 100%).

### EXAMPLE 77

### (S)-3-{4-[3-(4-Chloro-2-phenoxy-phenoxy)-pentyloxy]-2-methyl-phenyl}-propionic acid

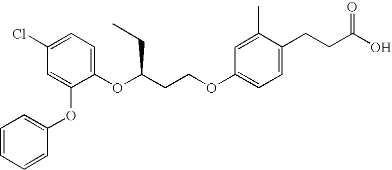

### (R)-3-[4-(3-Hydroxy-pentyloxy)-2-methyl-phenyl]-propionic acid methyl ester

### Step A

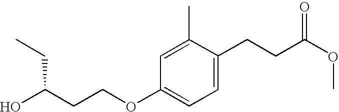

The compound of (R,S)-3-[4-(3-hydroxy-pentyloxy)-2-methyl-phenyl]-propionic acid methyl ester is purified by HPLC using a 4.6×250 mm Chiralpak AD column. The chiral pure compound is eluted using May 5, 1990 NPA/methanol/heptane. The solvent is removed to afford the desired product (95.7% ee). ¹H NMR (400 MHz, CDCl₃); MS (ES⁺) m/z mass calcd for C₁₆H₂₄O₄ 280, found 281 (M+1, 100%).

### Step B

### (R)-3-[4-(3-Methanesulfonyloxy-pentyloxy)-2-methyl-phenyl]-propionic acid methyl ester

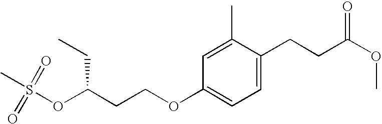

The procedure for Example 75, Step D is utilized with (R)-3-[4-(3-hydroxy-pentyloxy)-2-methyl-phenyl]-propionic acid methyl ester to afford 0.25 g (quantitative) of the desired product. ¹H NMR (400 MHz, CDCl₃); MS (ES⁺) m/z mass calcd for C₁₇H₂₆O₆S 358, found 359 (M+1, 100%).

### Step C

### (S)-3-{4-[3-(4-Chloro-2-phenoxy-phenoxy)-pentyloxy]-2-methyl-phenyl}-propionic acid

The procedure for Example 75, Step E is utilized with (R)-3-[4-(3-methanesulfonyloxy-pentyloxy)-2-methyl-phenyl]-propionic acid methyl ester to afford 66 mg (44%) of the desired product. ¹H NMR (400 MHz, CDCl₃); MS (ES⁺) m/z mass calcd for C₂₇H₂₉ClO₅ 468, found 469 (M+1, 100%).

### EXAMPLE 78

### (S)-3-{4-[3-(2-Benzoyl-4-ethyl-phenoxy)-pentyloxy]-2-methyl-phenyl}-propionic acid

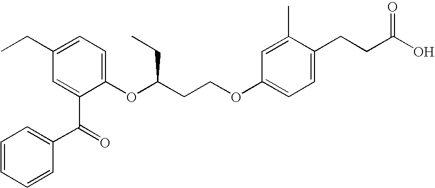

The procedure from Example 77, Step C is utilized with (5-ethyl-2-hydroxy-phenyl)-phenyl-methanone to afford 77 mg (50%) of the desired product. ¹H NMR (400 MHz, CDCl₃); MS (ES⁺) m/z mass calcd for C₃₀H₃₄O₅ 474, found 475 (M+1, 100%).

### EXAMPLE 79

### (R)-3-{4-[3-(3-Benzoyl-naphthalen-2-yloxy)-butoxy]-2-methyl-phenyl}-propionic acid

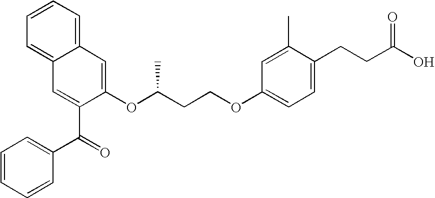

### Step A

### (3-Hydroxy-naphthalen-2-yl)-phenyl-methanone

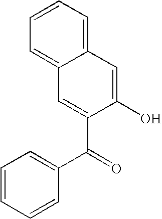

A solution of 3-hydroxy-2-napthoic acid (5.0 g, 26.6 mmol) in THF (200 mL) is cooled to −78° C. The solution is then treated dropwise with 1.8M phenyllithium in cyclohexane/ether (118 mL, 0.21 mol). The reaction is allowed to warm to rt and stir for 3 hours. The reaction is cooled and quenched with water. The reaction is neutralized to pH=6 with 1N aqueous hydrochloric acid, and extracted with ethyl ether. The organic is dried over sodium sulfate, filtered, and the solvent is removed to afford the crude product. The crude is purified by silica gel column chromatography using 9/1 hexanes/EtOAc to elute the pure product. The solvent is removed to afford 2.4 g (36%) of desired product. ¹H NMR (400 MHz, CDCl₃); MS (ES⁺) m/z mass calcd for C₁₇H₁₂O₂ 248, found 249 (M+1, 100%).

### Step B

### (R)-3-{4-[3-(3-Benzoyl-naphthalen-2-yloxy)-butoxy]-2-methyl-phenyl}-propionic acid methyl ester

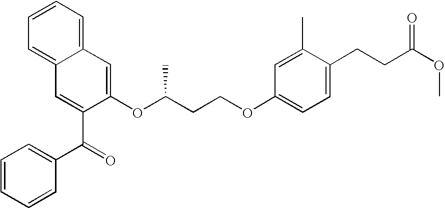

A solution of (3-hydroxy-naphthalen-2-yl)-phenyl-methanone (76 mg, 0.3 mmol) and (R)-3-[4-(3-methanesulfonyloxy-butoxy)-2-methyl-phenyl]-propionic acid methyl ester (0.1 g, 0.28 mmol) are combined in DMF (10 mL) and treated with cesium carbonate (0.109 g, 0.34 mmol). The reaction is heated to 60° C. and stirred overnight. The reaction is cooled and quenched with 1N aqueous hydrochloric acid. The aqueous is extracted with EtOAc. The organic is washed with brine, dried over sodium sulfate, filtered, and the solvent is removed to afford the crude product. The crude is purified by silica gel column chromatography using 9/1 Hexanes/EtOAc to elute the pure product. The solvent is removed to afford 99 mg (71%) of the desired product. ¹H NMR (400 MHz, CDCl₃); MS (ES⁺) m/z mass calcd for C₁₇H₂₆O₆S 496, found 497 (M+1, 100%).

### Step C

### (R)-3-{4-[3-(3-Benzoyl-naphthalen-2-yloxy)-butoxy]-2-methyl-phenyl}-propionic acid

The procedure from Example 71, Step C is utilized with (R)-3-{4-[3-(3-benzoyl-naphthalen-2-yloxy)-butoxy]-2-methyl-phenyl}-propionic acid methyl ester to afford 93 mg (quantitative) of desired product. ¹H NMR (400 MHz, CDCl₃); MS (ES⁺) m/z mass calcd for C₃₁H₃₀O₅ 482, found 483 (M+1, 100%).

### EXAMPLE 80

### (R)-{4-[3-(3-Benzoyl-naphthalen-2-yloxy)-butoxy]-2-methyl-phenylsulfanyl}-acetic acid

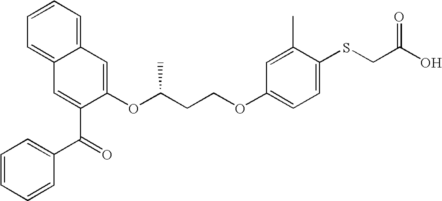

### Step A

### (R)-{4-[3-(3-Benzoyl-naphthalen-2-yloxy)-butoxy]-2-methyl-phenylsulfanyl}-acetic acid methyl ester

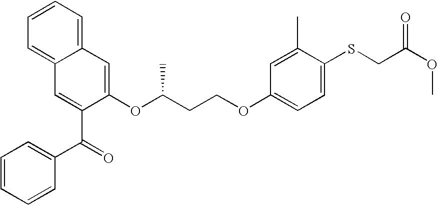

The procedure from Example 79, Step B is utilized with (R)-[4-(3-methanesulfonyloxy-butoxy)-2-methyl-phenylsulfanyl]-acetic acid methyl ester to afford 50 mg (36%) of the desired product. ¹H NMR (400 MHz, CDCl₃); MS (ES⁺) m/z mass calcd for C₃₁H₃₀O₅S 514, found 515 (M+1, 100%).

### Step B

### (R)-{4-[3-(3-Benzoyl-naphthalen-2-yloxy)-butoxy]-2-methyl-phenylsulfanyl}-acetic acid

The procedure from Example 71, Step C is utilized with (R)-{4-[3-(3-benzoyl-naphthalen-2-yloxy)-butoxy]-2-methyl-phenylsulfanyl}-acetic acid methyl ester to afford 47 mg (quantitative) of desired product. ¹H NMR (400 MHz, CDCl₃); MS (ES⁺) m/z mass calcd for C₃₀H₂₈O₅S 500, found 501 (M+1, 100%).

### EXAMPLE 81

### (R)-3-{4-[3-(4-Ethyl-2-phenoxy-phenoxy)-butylsulfanyl]-2-methyl-phenyl}-propionic acid

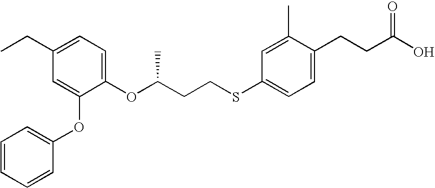

### Step A

### (R)-Methanesulfonic acid 3-(4-ethyl-2-phenoxy-phenoxy)-butyl ester

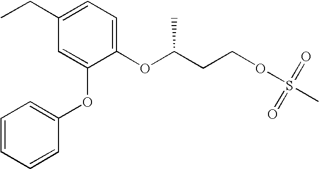

The procedure for Example 75, Step D is utilized with (R)-3-(4-ethyl-2-phenoxy-phenoxy)-butan-1-ol to afford 0.24 g (quantitative) of the desired product. ¹H NMR (400 MHz, CDCl₃); MS (ES⁺) m/z mass calcd for C₁₉H₂₄O₅S 364, found 365 (M+1, 100%).

### Step B

### (R)-3-{4-[3-(4-Ethyl-2-phenoxy-phenoxy)-butylsulfanyl]-2-methyl-phenyl}-propionic acid methyl ester

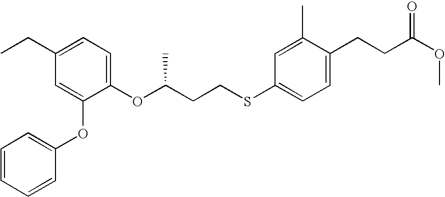

A solution of 3-(4-mercapto-2-methyl-phenyl)-propionic acid methyl ester (0.167 g, 0.8 mmol) in DMF (5 mL) is purged with nitrogen. The solution is treated with potassium carbonate (0.14 g, 1.0 mmol) and purged with nitrogen. The solution is then treated with (R)-methanesulfonic acid 3-(4-ethyl-2-phenoxy-phenoxy)-butyl ester (0.24 g, 0.66 mmol) and stirred overnight under nitrogen. The reaction is quenched with 1N aqueous hydrochloric acid. The aqueous is extracted with ethyl ether. The organic is washed with brine, dried over sodium sulfate, filtered, and the solvent removed to afford the crude product. The crude is purified by silica gel column chromatography using 9/1 Hexanes/Acetone to elute the pure product. The solvent is removed to afford 0.2 g (63%) of the desired product. ¹H NMR (400 MHz, CDCl₃); MS (ES⁺) m/z mass calcd for C₂₉H₃₄O₄S 478, found 479 (M+1, 100%).

### Step C

### (R)-3-{4-[3-(4-Ethyl-2-phenoxy-phenoxy)-butylsulfanyl]-2-methyl-phenyl}-propionic acid

The procedure for Example 71, Step C is utilized with (R)-3-{4-[3-(4-ethyl-2-phenoxy-phenoxy)-butylsulfanyl]-2-methyl-phenyl}-propionic acid methyl ester to afford 0.175 g (95%) of the desired product. ¹H NMR (400 MHz, CDCl₃); MS (ES⁺) m/z mass calcd for C₂₈H₃₂O₄S 464, found 465 (M+1, 100%).

### EXAMPLE 82

### (R)-3-{4-[3-(4-Isopropyl-2-phenoxy-phenoxy)-butylsulfanyl]-2-methyl-phenyl}-propionic acid

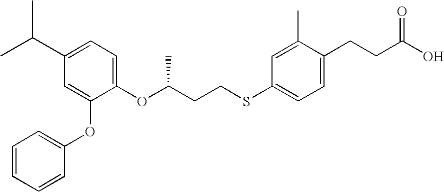

### Step A

### (R)-3-(4-Isopropyl-2-phenoxy-phenoxy)-butan-1-ol

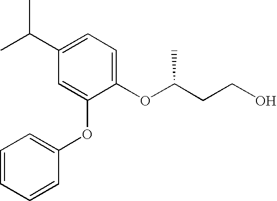

The procedure for Example 63, Step C is utilized with 4-isopropyl-2-phenoxy-phenol to afford 0.126 g (69%) of the desired product. ¹H NMR (400 MHz, CDCl₃); MS (ES⁺) m/z mass calcd for C₁₉H₂₄O₃ 300, found 301 (M+1, 100%).

### Step B

### (R)-Methanesulfonic acid 3-(4-Isopropyl-2-phenoxy-phenoxy)-butyl ester

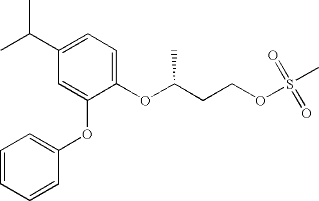

The procedure for Example 63, Step D is utilized with (R)-3-(4-isopropyl-2-phenoxy-phenoxy)-butan-1-ol to afford 0.100 g (63%) of the desired product. ¹H NMR (400 MHz, CDCl₃); MS (ES⁺) m/z mass calcd for C₂₀H₂₆O₅S 378, found 379 (M+1, 100%).

### Step C

### (R)-3-{4-[3-(4-Isopropyl-2-phenoxy-phenoxy)-butylsulfanyl]-2-methyl-phenyl}-propionic acid methyl ester

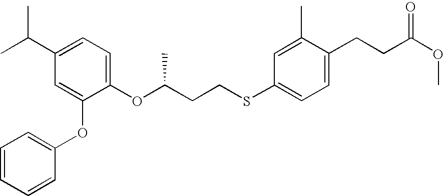

The procedure for Example 81, Step B is utilized with (R)-methanesulfonic acid 3-(4-Isopropyl-2-phenoxy-phenoxy)-butyl ester to afford 0.105 g (81%) of the desired product. ¹H NMR (400 MHz, CDCl₃); MS (ES⁺) m/z mass calcd for C₃₀H₃₆O₄S 492, found 493 (M+1, 100%).

### Step D

### (R)-3-{4-[3-(4-Isopropyl-2-phenoxy-phenoxy)-butylsulfanyl]-2-methyl-phenyl}-propionic acid

The procedure for Example 71, Step C is utilized with (R)-3-{4-[3-(4-Isopropyl-2-phenoxy-phenoxy)-butylsulfanyl]-2-methyl-phenyl}-propionic acid methyl ester to afford 0.091 g (89%) of the desired product. ¹H NMR (400 MHz, CDCl₃); MS (ES⁺) m/z mass calcd for C₂₉H₃₄O₄S 478, found 479 (M+1, 100%).

### EXAMPLE 83

### (R)-3-{4-[3-(2-Benzoyl-4,5-dichloro-phenoxy)-butoxy]-2-methyl-phenyl}-propionic acid

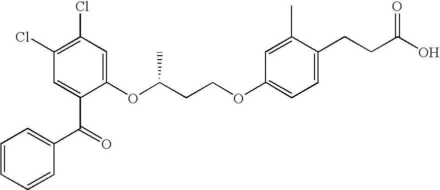

### Step A

### (S)-3-[4-(3-Hydroxy-butoxy)-2-methyl-phenyl]-propionic acid methyl ester

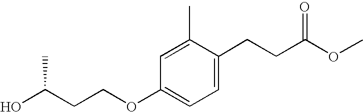

A solution of (S)-toluene-4-sulfonic acid 3-hydroxy-butyl ester (2.08 g, 8.5 mmol) and 3-(4-hydroxy-2-methyl-phenyl)-propionic acid methyl ester (1.5 g, 7.7 mmol) in DMF (20 mL) is treated with cesium carbonate (3.0 g, 9.3 mmol). The reaction is heated to 55° C. and stirred overnight. The reaction is cooled and quenched with 1N aqueous hydrochloric acid. The aqueous is extracted with ethyl ether. The organic is washed with brine, dried over sodium sulfate, filtered, and the solvent is removed to afford the crude product. The crude is purified by silica gel column chromatography using 9/1 hexanes/EtOAc to elute the pure product. The solvent is removed to afford 0.67 g (33%) of the desired product. ¹H NMR (400 MHz, CDCl₃); MS (ES⁺) m/z mass calcd for C₁₅H₂₂O₄ 266, found 267 (M+1, 100%).

### Step B

### (S)-3-[4-(3-Methanesulfonyloxy-butoxy)-2-methyl-phenyl]-propionic acid methyl ester

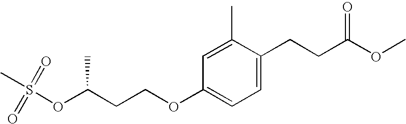

The procedure for Example 63, Step D is utilized with (S)-3-[4-(3-hydroxy-butoxy)-2-methyl-phenyl]-propionic acid methyl ester to afford 0.87 g (quantitative) of the desired product. ¹H NMR (400 MHz, CDCl₃); MS (ES⁺) m/z mass calcd for C₁₆H₂₄O₆S 344, found 345 (M+1, 100%).

### Step C

### (R)-3-{4-[3-(2-Benzoyl-4,5-dichloro-phenoxy)-butoxy]-2-methyl-phenyl}-propionic acid

A solution of (S)-3-[4-(3-methanesulfonyloxy-butoxy)-2-methyl-phenyl]-propionic acid methyl ester (0.1 g, 0.29 mmol) and (4,5-dichloro-2-hydroxy-phenyl)-phenyl-methanone (85 mg, 0.32 mmol) in DMF (3 mL) is treated with cesium carbonate (113 mg, 0.35 mmol). The reaction is heated to 60° C. and stirred overnight. The reaction is treated with aqueous 5N NaOH (0.6 mL, 2.9 mmol) and stirred for 2 additional hours at 60° C. The reaction is cooled and quenched with 1N HCl to pH=4. The reaction is extracted with Et₂O. The organic is washed with brine, dried over sodium sulfate, filtered, and the solvent is removed. The crude product is purified by prep HPLC to afford 48 mg (33%) of the desired product. ¹H NMR (400 MHz, CDCl₃); MS (ES⁺) m/z mass calcd for C₂₇H₂₆Cl₂O₅ 500, found 501 (M+1, 100%).

### EXAMPLE 84

### (R)-3-{2-Ethyl-4-[3-(4-ethyl-2-phenoxy-phenoxy)-butoxy]-phenyl}-propionic acid

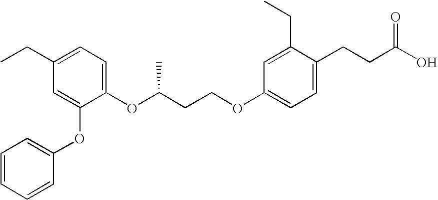

### Step A

### (S)-3-[2-Ethyl-4-(3-hydroxy-butoxy)-phenyl]-propionic acid ethyl ester

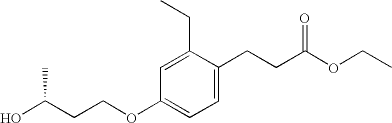

The procedure from Example 83, Step A is utilized with 3-(2-ethyl-4-hydroxy-phenyl)-propionic acid methyl ester to afford 1.12 g (56%) of the desired product. ¹H NMR (400 MHz, CDCl₃); MS (ES⁺) m/z mass calcd for C₁₇H₂₆O₄ 294, found 295 (M+1, 100%).

### Step B

### (S)-3-[2-Ethyl-4-(3-methanesulfonyloxy-butoxy)-phenyl]-propionic acid ethyl ester

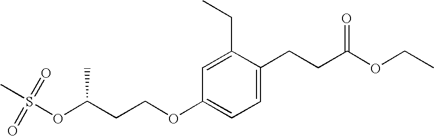

The procedure for Example 63, Step D is utilized with (S)-3-[2-Ethyl-4-(3-hydroxy-butoxy)-phenyl]-propionic acid ethyl ester to afford 1.17 g (84%) of the desired product. ¹H NMR (400 MHz, CDCl₃); MS (ES⁺) m/z mass calcd for C₁₈H₂₈O₆S 372, found 373 (M+1, 100%).

### Step C

### (R)-3-{2-Ethyl-4-[3-(4-ethyl-2-phenoxy-phenoxy)-butoxy]-phenyl}-propionic acid

A solution of (S)-3-[2-ethyl-4-(3-methanesulfonyloxy-butoxy)-phenyl]-propionic acid ethyl ester (0.1 g, 0.27 mmol) and 4-ethyl-2-phenoxy-phenol (64 mg, 0.3 mmol) in DMF (3 mL) is treated with cesium carbonate (105 mg, 0.32 mmol). The reaction is heated to 60° C. and stirred overnight. The reaction is then treated with aqueous 5N NaOH (0.6 mL, 2.9 mmol) and stirred for 2 additional hours at 60° C. The reaction is cooled and quenched with 1N HCl to pH=4. The reaction is extracted with Et2O. The organic is washed with brine, dried over sodium sulfate, filtered, and the solvent is removed. The crude product is purified by prep HPLC to afford 60 mg (48%) of the desired product. ¹H NMR (400 MHz, CDCl₃); MS (ES⁺) m/z mass calcd for C₂₉H₃₄O₅ 462, found 463 (M+1, 100%).

### EXAMPLE 85

### (R)-3-{2-Ethyl-4-[3-(2-phenoxy-4-trifluoromethyl-phenoxy)-butoxy]-phenyl}-propionic acid

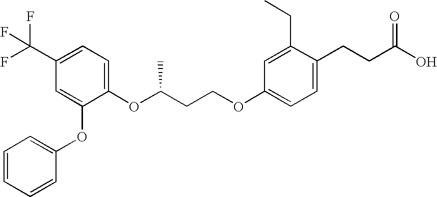

The procedure from Example 84, Step C is utilized with 2-phenoxy-4-trifluoromethyl-phenol to afford 63 mg (47%) of the desired product. ¹H NMR (400 MHz, CDCl₃); MS (ES⁺) m/z mass calcd for C₂₈H₂₉F₃O₅ 502, found 503 (M+1, 100%).

### EXAMPLE 86

### (R)-3-{4-[3-(2-Benzoyl-4-ethyl-phenoxy)-butoxy]-2-ethyl-phenyl}-propionic acid

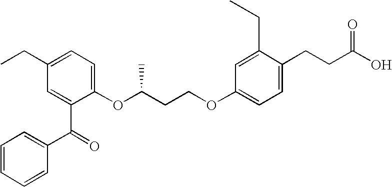

The procedure from Example 84, Step C is utilized with (5-ethyl-2-hydroxy-phenyl)-phenyl-methanone to afford 63 mg (49%) of the desired product. ¹H NMR (400 MHz, CDCl₃); MS (ES⁺) m/z mass calcd for C₃₀H₃₄O₅ 474, found 475 (M+1, 100%).

### EXAMPLE 87

### (R)-3-{4-[3-(2,4-Diphenoxy-phenoxy)-butoxy]-2-ethyl-phenyl}-propionic acid

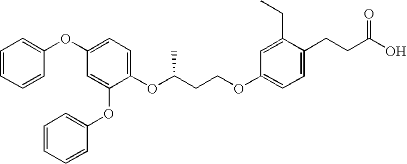

The procedure from Example 84, Step C is utilized with 2,4-diphenoxy-phenol to afford 105 g (50%) of the desired product. ¹H NMR (400 MHz, CDCl₃); MS (ES⁺) m/z mass calcd for C₃₃H₃₄O₆ 526, found 527 (M+1, 100%).

### EXAMPLE 88

### (R)-3-{2-Methyl-4-[3-(2-phenoxy-4-trifluoromethyl-phenoxy)-butylsulfanyl]-phenyl}-propionic acid

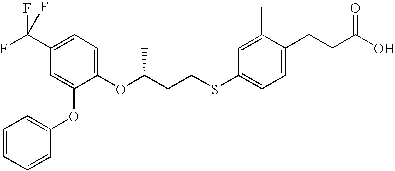

### Step A

### (S)-3-[4-(3-Hydroxy-butylsulfanyl)-2-methyl-phenyl]-propionic acid methyl ester

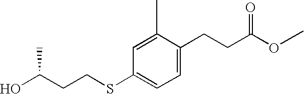

A solution of 3-(4-mercapto-2-methyl-phenyl)-propionic acid methyl ester (1.0 g, 7.1 mmol) in DMF (20 mL) is purged with nitrogen. The solution is treated with potassium carbonate (1.48 g, 10.7 mmol) and purged again with nitrogen. The reaction is then treated with (S)-toluene-4-sulfonic acid 3-hydroxy-butyl ester (1.28 g, 7.9 mmol) and stirred overnight at rt under nitrogen. The reaction is quenched with 1N aqueous hydrochloric acid. The aqueous is extracted with ethyl ether. The organic is washed with brine, dried over sodium sulfate, filtered, and the solvent is removed to afford the crude product. The crude is purified by silica gel column chromatography using 9/1 hexanes/acetone to elute the pure product. The solvent is removed to afford 0.96 g (72%) of the desired product. ¹H NMR (400 MHz, CDCl₃); MS (ES⁺) m/z mass calcd for C₁₅H₂₂O₃S 282, found 283 (M+1, 100%).

### Step B

### (S)-3-[4-(3-Methanesulfonyloxy-butylsulfanyl)-2-methyl-phenyl]-propionic acid methyl ester

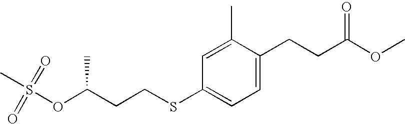

The procedure for Example 63, Step D is utilized with (S)-3-[4-(3-hydroxy-butylsulfanyl)-2-methyl-phenyl]-propionic acid methyl ester to afford 1.2 g (quantitative) of the desired product. ¹H NMR (400 MHz, CDCl₃); MS (ES⁺) m/z mass calcd for C₁₆H₂₄O₅S₂ 360, found 361 (M+1, 100%).

### Step C

### (R)-3-{2-Methyl-4-[3-(2-phenoxy-4-trifluoromethyl-phenoxy)-butylsulfanyl]-phenyl}-propionic acid

A solution of (S)-3-[4-(3-methanesulfonyloxy-butylsulfanyl)-2-methyl-phenyl]-propionic acid methyl ester (0.1 g, 0.28 mmol) and 2-phenoxy-4-trifluoromethyl-phenol (78 mg, 0.31 mmol) in DMF (3 mL) is treated with cesium carbonate (108 mg, 0.33 mmol). The reaction is heated to 60° C. and stirred overnight. The reaction is then treated with aqueous 5N NaOH (0.6 mL, 2.9 mmol) and stirred for 2 additional hours at 60° C. The reaction is then cooled and quenched with 1N HCl to pH=4. The reaction is then extracted with Et₂O. The organic is washed with brine, dried over sodium sulfate, filtered, and the solvent is removed. The crude product is purified by prep HPLC to afford 18 mg (13%) of the desired product. ¹H NMR (400 MHz, CDCl₃); MS (ES⁺) m/z mass calcd for C₂₇H₂₇F₃O₅S 504, found 505 (M+1, 100%).

### EXAMPLE 89

### (R)-3-{4-[3-(4-Ethyl-2-phenoxy-phenoxy)-butoxy]-2,6-dimethyl-phenyl}-propionic acid

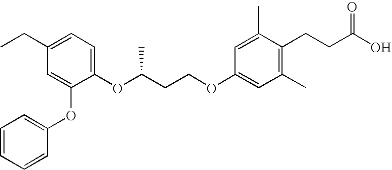

### Step A

### (R)-3-(4-Ethyl-2-phenoxy-phenoxy)-butan-1-ol

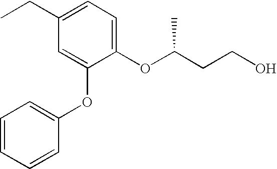

The procedure from Example 63, Step C is utilized with 4-ethyl-2-phenoxy-phenol to afford 0.52 g (78%) of the desired product. ¹H NMR (400 MHz, CDCl₃); MS (ES⁺) m/z mass calcd for C₁₈H₂₂O₃ 286, found 287 (M+1, 100%).

### Step B

### (R)-Methanesulfonic acid 3-(4-ethyl-2-phenoxy-phenoxy)-butyl ester

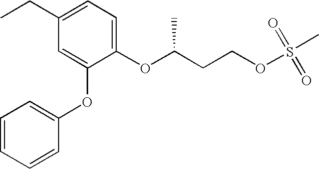

The procedure from Example 63, Step D is utilized with (R)-3-(4-ethyl-2-phenoxy-phenoxy)-butan-1-ol to afford 0.64 g (96%) of the desired product. ¹H NMR (400 MHz, CDCl₃); MS (ES⁺) m/z mass calcd for C₁₉H₂₄O₅S 364, found 365 (M+1, 100%).

### Step C

### (R)-3-{4-[3-(4-Ethyl-2-phenoxy-phenoxy)-butoxy]-2,6-dimethyl-phenyl}-propionic acid

A solution of (R)-methanesulfonic acid 3-(4-ethyl-2-phenoxy-phenoxy)-butyl ester (0.1 g, 0.27 mmol) and 3-(4-hydroxy-2,6-dimethyl-phenyl)-propionic acid methyl ester (67 mg, 0.3 mmol) in DMF (5 mL) is treated with cesium carbonate (107 mg, 0.33 mmol). The reaction is heated to 50° C. and stirred overnight. The reaction is then treated with aqueous 5N NaOH (0.54 mL, 2.7 mmol) and stirred for 2 additional hours at 50° C. The reaction is then cooled and quenched with 1N HCl to pH=4. The reaction is then extracted with Et2O. The organic is washed with brine, dried over sodium sulfate, filtered, and the solvent is removed. The crude product is purified by prep HPLC to afford 31 mg (25%) of the desired product. ¹H NMR (400 MHz, CDCl₃); MS (ES⁺) m/z mass calcd for C₂₉H₃₄O₅ 462, found 463 (M+1, 100%).

### EXAMPLE 90

### (R)-3-{2-Ethyl-4-[1-methyl-3-(2-phenoxy-4-trifluoromethyl-phenoxy)-propoxy]-phenyl}-propionic acid

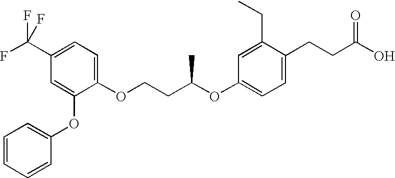

### Step A

### (S)-4-(2-Phenoxy-4-trifluoromethyl-phenoxy)-butan-2-ol

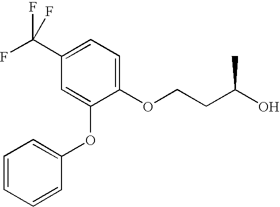

A mixture of 2-phenoxy-4-trifluoromethyl-phenol (502 mg, 1.97 mmol), (S)-toluene-4-sulfonic acid 3-hydroxy-butyl ester (531 mg, 2.17 mmol) and Cs₂CO₃ (965 mg, 2.96 mmol) in 20 mL of dry DMF is heated to 55° C. for overnight. The mixture is then cooled to rt and diluted with Et₂O and filtered through a pad of celite. Organic layer is washed with 1N HCl, H₂O, brine and dried over Na₂SO₄, filtered and concentrated. Crude material is purified by chromatography (hexanes/acetone=8:1) to afford the title compound as a colorless oil in 79% yield. R_{f}=0.31 (8/1 hexanes/acetone). ¹H NMR (400 MHz, CDCl₃).

### Step B

### (S)-methanesulfonic acid 1-methyl-3-(2-phenoxy-4-trifluoromethyl-phenoxy)-propyl ester

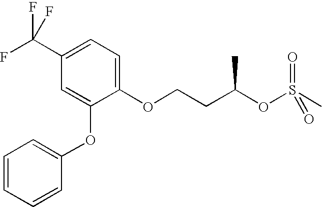

A mixture of (S)-4-(2-phenoxy-4-trifluoromethyl-phenoxy)-butan-2-ol (360 mg, 1.10 mmol), mathanesulfonyl chloride (0.13 mL, 1.65 mmol) and Et₃N (0.38 mL, 2.76 mmol) in 15 mL of dry CH₂Cl₂ is allowed to stand at 0° C. for 30 min and then slowly warm up to rt for 2 h. The mixture is then diluted with Et₂O and is washed with 1N HCl, H₂O, brine and dried over Na₂SO₄, filtered and concentrated. The crude material is used for next step without further purification. R_{f}=0.3 (15/1 hexanes/acetone). ¹H NMR (400 MHz, CDCl₃).

### Step C

### (R)-3-{2-Ethyl-4-[1-methyl-3-(2-phenoxy-4-trifluoromethyl-phenoxy)-propoxy]-phenyl}-propionic acid

A solution of (R)-methanesulfonic acid 1-methyl-3-(2-phenoxy-4-trifluoromethyl-phenoxy)-propyl ester (0.1 g, 0.25 mmol) and 3-(2-ethyl-4-hydroxy-phenyl)-propionic acid ethyl ester (60 mg, 0.27 mmol) in DMF (3 mL) is treated with cesium carbonate (98 mg, 0.3 mmol). The reaction is heated to 50° C. and stirred overnight. The reaction is treated with aqueous 5N NaOH (0.5 mL, 2.7 mmol) and stirred for 2 additional hours at 50° C. The reaction is cooled and quenched with 1N HCl to pH=4. The reaction is extracted with Et2O. The organic is washed with brine, dried over sodium sulfate, filtered, and the solvent is removed. The crude product is purified by prep HPLC to afford 27 mg (21%) of the desired product. ¹H NMR (400 MHz, CDCl₃); MS (ES⁺) m/z mass calcd for C₂₈H₂₉F₃O₅ 502, found 503 (M+1, 100%).

### EXAMPLE 91

### (R)-3-{2-Methyl-4-[1-methyl-3-(2-phenoxy-4-trifluoromethyl-phenoxy)-propylsulfanyl]-phenyl}-propionic acid

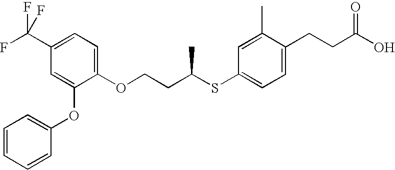

### Step A

### (S)-4-(2-Phenoxy-4-trifluoromethyl-phenoxy)-butan-2-ol

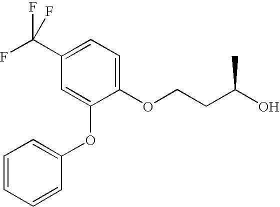

A mixture of 2-phenoxy-4-trifluoromethyl-phenol (502 mg, 1.97 mmol), (S)-toluene-4-sulfonic acid 3-hydroxy-butyl ester (531 mg, 2.17 mmol) and Cs₂CO₃ (965 mg, 2.96 mmol) in 20 mL of dry DMF is heated to 55° C. for overnight. The mixture is then cooled to rt and diluted with Et₂O and filtered through a pad of celite. Organic layer is washed with 1N HCl, H₂O, brine and dried over Na₂SO₄, filtered and concentrated. Crude material is purified by chromatography (hexanes/acetone=8:1) to afford the title compound as a colorless oil in 79% yield. R_{f}=0.31 (8/1 hexanes/acetone). ¹H NMR (400 MHz, CDCl₃).

### Step B

### (S)-methanesulfonic acid 1-methyl-3-(2-phenoxy-4-trifluoromethyl-phenoxy)-propyl ester

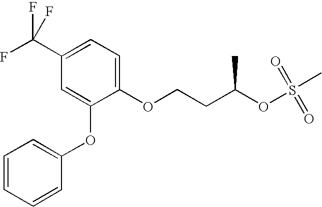

A mixture of (S)-4-(2-phenoxy-4-trifluoromethyl-phenoxy)-butan-2-ol (360 mg, 1.10 mmol), mathanesulfonyl chloride (0.13 mL, 1.65 mmol) and Et₃N (0.38 mL, 2.76 mmol) in 15 mL of dry CH₂Cl₂ is allowed to stand at 0° C. for 30 min and then slowly warm up to rt for 2 h. The mixture is then diluted with Et₂O and is washed with 1N HCl, H₂O, brine and dried over Na₂SO₄, filtered and concentrated. The crude material is used for next step without further purification. R_{f}=0.3 (15/1 hexanes/acetone). ¹H NMR (400 MHz, CDCl₃).

### Step C

### (R)-3-{2-Methyl-4-[1-methyl-3-(2-phenoxy-4-trifluoromethyl-phenoxy)-propylsulfanyl]-phenyl}-propionic acid

A solution of methanesulfonic acid 1-methyl-3-(2-phenoxy-4-trifluoromethyl-phenoxy)-propyl ester (0.1 g, 0.25 mmol) in DMF (5 mL) is purged with nitrogen. The solution is treated with potassium carbonate (51 mg, 0.37 mmol) and purged again with nitrogen. The solution is then treated with 3-(4-Mercapto-2-methyl-phenyl)-propionic acid methyl ester (57 mg, 0.27 mmol) and stirred at rt overnight. The reaction is quenched with 1N aqueous hydrochloric acid. The aqueous is extracted with ethyl ether. The organic is washed with brine, dried over sodium sulfate, filtered, and the solvent removed to afford the crude product. The crude is purified by silica gel column chromatography using 9/1 hexanes/acetone to elute the methyl ester intermediate. The intermediate is treated with 5N NaOH (0.5 mL, 2.5 mmol) in MeOH (5 mL) and heated to reflux. The reaction stirred at reflux for 2 hours and then is cooled. The reaction is quenched with 1N aqueous hydrochloric acid to pH=4. The aqueous is extracted with ethyl ether. The organic is washed with brine, dried over sodium sulfate, and filtered. The solvent is removed to afford 0.032 g (26%) of desired product. ¹H NMR (400 MHz, CDCl₃); MS (ES) m/z mass calcd for C₂₇H₂₇F₃O₄S 504, found 505 (M+1, 100%).

### EXAMPLE 92

### (R)-3-{2-Methyl-4-[1-methyl-3-(2-phenoxy-4-trifluoromethoxy-phenoxy)-propylsulfanyl]-phenyl}-propionic acid

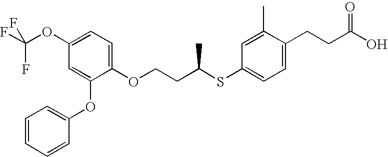

### Step A

### (S)-4-(2-Bromo-4-trifluoromethoxy-phenoxy)-butan-2-ol

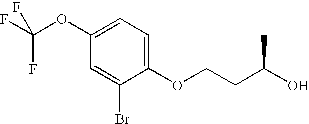

A mixture of 2-bromo-4-trifluoromethoxy-phenol (1.0 g, 3.9 mmol), (S)-toluene-4-sulfonic acid 3-hydroxy-butyl ester (1.05 g, 4.3 mmol) and Cs₂CO₃ (1.9 g, 5.8 mmol) in 20 mL of dry DMF is heated to 60° C. for overnight. The mixture is then cooled to rt and diluted with Et₂O and filtered through a pad of celite. Organic layer is washed with 1N HCl, H₂O, brine and dried over Na₂SO₄, filtered and concentrated. Crude material is purified by chromatography (hexanes/acetone=15:1) to afford the title compound as a colorless oil in 83% yield. R_{f}=0.3 (15/1 hexanes/acetone). ¹H NMR (400 MHz, CDCl₃).

### Step B

### (S)-Methanesulfonic acid 3-(2-bromo-4-trifluoromethoxy-phenoxy)-1-methyl-propyl ester

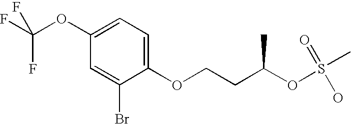

A mixture of (S)-4-(2-bromo-4-trifluoromethoxy-phenoxy)-butan-2-ol (900 mg, 2.73 mmol), mathanesulfonyl chloride (0.32 mL, 4.10 mmol) and Et₃N (0.95 mL, 6.84 mmol) in 30 mL of dry CH₂Cl₂ is allowed to stand at 0° C. for 30 min and then slowly warm up to rt for 2 h. The mixture is then diluted with Et₂O and is washed with 1N HCl, H₂O, brine and dried over Na₂SO₄, filtered and concentrated. The crude material is used for next step without further purification. R_{f}=0.33 (13/1 hexanes/acetone). ¹H NMR (400 MHz, CDCl₃).

### Step C

### (R)-3-{4-[3-(2-Bromo-4-trifluoromethoxy-phenoxy)-1-methyl-propylsulfanyl]-2-methyl-phenyl}-propionic acid methyl ester

A mixture of (S)-methanesulfonic acid 3-(2-bromo-4-trifluoromethoxy-phenoxy)-1-methyl-propyl ester (210 mg, 0.52 mmol), 3-(4-mercapto-2-methyl-phenyl)-propionic acid methyl ester (90.4 mg, 0.43 mmol) and K₂CO₃ (89.1 mg, 0.65 mmol) in 10 mL of dry DMF is allowed to stand at rt for overnight. The mixture is diluted with Et₂O and filtered through a pad of celite. Organic layer is washed with 1N HCl, H₂O, brine and dried over Na₂SO₄, filtered and concentrated. Crude material is purified by chromatography (hexanes/acetone=10:1) to afford the title compound as a colorless oil in 83% yield. R_{f}=0.26 (10/1 hexanes/acetone). ¹H NMR (400 MHz, CDCl₃).

### Step D

### (R)-3-{2-Methyl-4-[1-methyl-3-(2-phenoxy-4-trifluoromethoxy-phenoxy)-propylsulfanyl]-phenyl}-propionic acid

A solution of 3-{4-[3-(2-bromo-4-trifluoromethoxy-phenoxy)-1-methyl-propylsulfanyl]-2-methyl-phenyl}-propionic acid methyl ester (0.117 g, 0.22 mmol), phenol (63 mg, 0.67 mmol), copper(II) chloride (11 mg, 0.11 mmol), 2,2,6,6-Tetramethyl-3,5-heptanedione (5 mg, 0.03 mmol), and cesium carbonate (0.219 g, 0.67 mmol) in NMP (5 mL) is heated to 120° C. The reaction stirred overnight, and then is cooled to room temperature. The reaction is then quenched with 1N aqueous hydrochloric acid and extracted with ethyl ether. The organic is washed with brine, dried over sodium sulfate, and filtered. The solvent is removed to afford the crude ester intermediate. The intermediate is treated with 5N NaOH (0.4 mL, 2.2 mmol) in MeOH (5 mL) and heated to reflux. The reaction stirred at reflux for 2 hours and then is cooled. The reaction is quenched with 1N aqueous hydrochloric acid to pH=4. The aqueous is extracted with ethyl ether. The organic is washed with brine, dried over sodium sulfate, and filtered. The solvent is removed to afford the crude product. The crude is purified by prep HPLC to afford 30 mg (26%) of desired product. ¹H NMR (400 MHz, CDCl₃); MS (ES⁺) m/z mass calcd for C₂₇H₂₇F₃O₅S 520, found 521 (M+1, 100%).

### EXAMPLE 93

### (S)-3-{4-[3-(4-Chloro-2-phenoxy-phenoxy)-butoxy]-2-ethyl-phenyl}-propionic acid

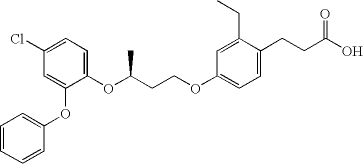

### Step A

### (S)-3-(4-Chloro-2-phenoxy-phenoxy)-butan-1-ol

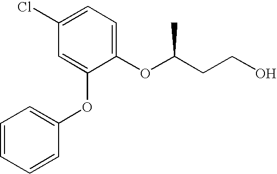

A solution of (R)-acetic acid 3-(toluene-4-sulfonyloxy)-butyl ester (1.43 g, 5 mmol) and 4-chloro-2-phenoxy-phenol (1.0 g, 4.5 mmol) in DMF (20 mL) is treated with cesium carbonate (1.77 g, 5.4 mmol). The solution is heated to 60° C. and stirred overnight. The reaction is cooled and quenched with 1N HCl. The solution is partitioned in EtOAc and water. The organic is separated, washed with brine, and dried over sodium sulfate. The organic is filtered and the solvent is removed to afford acetic acid 3-(4-chloro-2-phenoxy-phenoxy)-butyl ester, which is then diluted in methanol (20 mL) and treated with potassium carbonate (1.5 g, 10.9 mmol). The reaction is stirred for 3 hours at room temperature. The reaction is partitioned in ethyl ether and water. The organic layer is separated, washed with brine, and dried over sodium sulfate. The organic is filtered and the solvent is removed to afford the crude product. The crude is purified by silica gel column chromatography using 1/1 hexanes/EtOAc to elute the pure product. The solvent is removed to afford 0.99 g (88%) of the desired product. ¹H NMR (400 MHz, CDCl₃); MS (ES⁺) m/z mass calcd for C₁₆H₁₇ClO₃ 292, found 293 (M+1, 100%).

### Step B

### (S)-Methanesulfonic acid 3-(4-chloro-2-phenoxy-phenoxy)-butyl ester

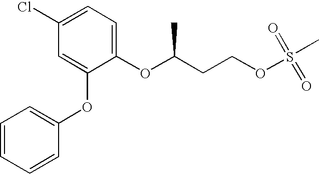

A solution of (S)-3-(4-chloro-2-phenoxy-phenoxy)-butan-1-ol (0.99 g, 3.2 mmol) in CH₂Cl₂ (20 mL) is cooled to 0° C. The solution is then treated with TEA (0.38 g, 3.8 mmol) and MsCl (0.44 g, 3.8 mmol). The reaction stirred for 2 hours at 0° C. The reaction is diluted in water and extracted with CH₂Cl₂. The organic is separated, washed with brine, and dried over sodium sulfate. The organic is filtered and the solvent is removed to afford 1.28 g (100%) of the desired product. ¹H NMR (400 MHz, CDCl₃); MS (ES⁺) m/z mass calcd for C₁₇H₁₉ClO₅S 370, found 371 (M+1, 100%).

### Step C

### (S)-3-{4-[3-(4-Chloro-2-phenoxy-phenoxy)-butoxy]-2-ethyl-phenyl}-propionic acid

A solution of (S)-methanesulfonic acid 3-(4-chloro-2-phenoxy-phenoxy)-butyl ester (0.15 g, 0.4 mmol) and 3-(2-chloro-4-hydroxy-phenyl)-propionic acid ethyl ester (0.099 g, 0.44 mmol) in DMF (5 mL) is treated with cesium carbonate (0.158 g, 0.49 mmol). The reaction is heated to 50° C. and stirred overnight. The reaction is cooled and quenched with 1N HCl. The solution is partitioned in EtOAc and water. The organic is separated, washed with brine, and dried over sodium sulfate. The organic is filtered and the solvent removed to afford the crude product. The crude is purified by silica gel column chromatography using 9/1 hexanes/EtOAc to elute the pure product. The solvent is removed to afford (S)-3-{4-[3-(4-chloro-2-phenoxy-phenoxy)-butoxy]-2-ethyl-phenyl}-propionic acid ethyl ester. This intermediate is treated with 5N aqueous sodium hydroxide in ethanol and heated to reflux. The reaction stirred for 3 hours and then is cooled to rt. The reaction is quenched with 1N aqueous hydrochloric acid and pH adjusted to pH=3. The aqueous is extracted with ether and washed with brine. The organic is dried over sodium sulfate, filtered, and the solvent is removed to afford 0.096 g (51%) of desired product. ¹H NMR (400 MHz, CDCl₃); MS (ES⁺) m/z mass calcd for C₂₇H₂₉ClO₅ 468, found 469 (M+1, 100%).

### EXAMPLE 94

### 3-{4-[3-(4-Chloro-2-phenoxy-phenoxy)-propoxy]-2-ethyl-phenyl}-propionic acid

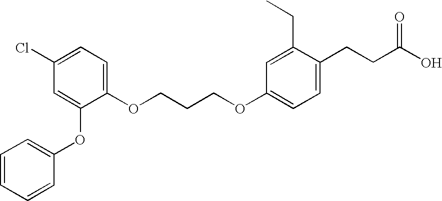

### Step A

### 3-(4-Chloro-2-phenoxy-phenoxy)-propan-1-ol

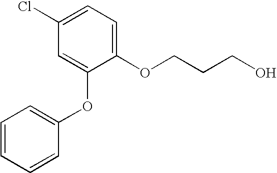

The procedure from Example 93, Step A is utilized with 3-bromo-1-propanol to afford 0.3 g (48%) of desired product. ¹H NMR (400 MHz, CDCl₃); MS (ES⁺) m/z mass calcd for C₁₅H₁₅ClO₃ 278, found 279 (M+1, 100%).

### Step B

### Methanesulfonic acid 3-(4-chloro-2-phenoxy-phenoxy)-propyl ester

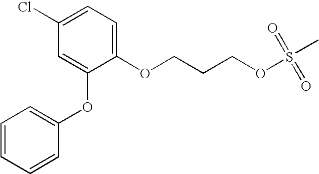

The procedure for Example 93, Step B is utilized with 3-(4-chloro-2-phenoxy-phenoxy)-propan-1-ol to afford 0.319 g (83%) of the desired product. ¹H NMR (400 MHz, CDCl₃); MS (ES⁺) m/z mass calcd for C₁₆H₁₇ClO₅S 356, found 357 (M+1, 100%).

### Step C

### 3-{4-[3-(4-Chloro-2-phenoxy-phenoxy)-propoxy]-2-ethyl-phenyl}-propionic acid ethyl ester

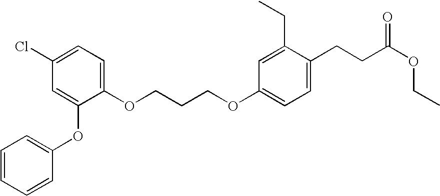

A solution of mthanesulfonic acid 3-(4-chloro-2-phenoxy-phenoxy)-propyl ester (0.319 g, 0.9 mmol) and 3-(2-ethyl-4-hydroxy-phenyl)-propionic acid ethyl ester (0.218 g, 0.98 mmol) in DMF (10 mL) is treated with cesium carbonate (0.349 g, 1.07 mmol). The reaction is heated to 60° C. and stirred overnight. The reaction is cooled and quenched with 1N aqueous hydrochloric acid. he solution is partitioned in ethyl ether and water. The organic is separated, washed with brine, and dried over sodium sulfate. The organic is filtered and the solvent is removed to afford the crude product. The crude is purified by silica gel column chromatography using 9/1 hexanes/EtOAc to elute the pure product. The solvent is removed to afford 0.337 g (78%) of the desired product. ¹H NMR (400 MHz, CDCl₃); MS (ES⁺) m/z mass calcd for C₂₈H₃₁ClO₅ 482, found 483 (M+1, 100%).

### Step D

### 3-{4-[3-(4-Chloro-2-phenoxy-phenoxy)-propoxy]-2-ethyl-phenyl}-propionic acid

A solution of 3-{4-[3-(4-chloro-2-phenoxy-phenoxy)-propoxy]-2-ethyl-phenyl}-propionic acid ethyl ester (0.337 g, 0.7 mmol) in ethanol (10 mL) is treated with 5N aqueous sodium hydroxide (1.4 mL). The reaction is heated to reflux and stirred for 2 hours. The reaction is then cooled and the pH adjusted to pH=4 with 1N aqueous hydrochloric acid. The solution is extracted with EtOAc. The organic is washed with brine, dried over sodium sulfate, filtered, and the solvent is removed to afford 0.28 g (88%) of desired product. ¹H NMR (400 MHz, CDCl₃); MS (ES⁺) m/z mass calcd for C₂₆H₂₇ClO₅ 454, found 455 (M+1, 100%).

### EXAMPLE 95

### 2-{4-[4-(4-Chloro-2-phenoxy-phenyl)-3-methyl-butoxy]-2-methyl-phenyl}-cyclopropanecarboxylic acid

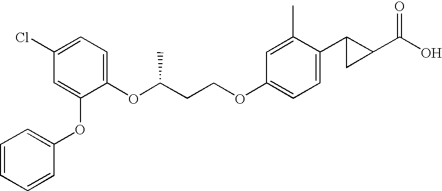

### Step A

### 2-(4-Hydroxy-2-methyl-phenyl)-cyclopropanecarboxylic acid ethyl ester

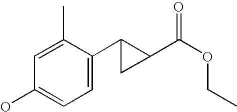

A solution of 2-(4-benzyloxy-2-methyl-phenyl)-cyclopropanecarboxylic acid ethyl ester (2.0 g, 6.75 mmol) in EtOAc (100 mL) is treated with 10% Palladium on carbon (0.5 g) and stirred under hydrogen (1 atm). The reaction stirred for 3 hours. The reaction is filtered through celite, and the filtrate is concentrated to afford 1.3 g (94%) of title compound. ¹H NMR (400 MHz, CDCl₃); MS (ES⁺) m/z mass calcd for C₂₀H₂₂O₃ 310, found 311 (M+1, 100%).

### Step B

### 2-{4-[4-(4-Chloro-2-phenoxy-phenyl)-3-methyl-butoxy]-2-methyl-phenyl}-cyclopropanecarboxylic acid ethyl ester

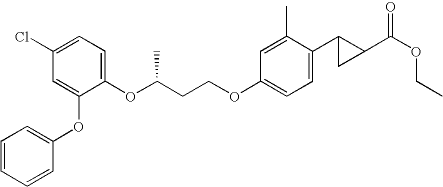

A solution of (R)-methanesulfonic acid 3-(4-chloro-2-phenoxy-phenoxy)-butyl ester (0.8 g, 2.16 mmol) and 2-(4-hydroxy-2-methyl-phenyl)-cyclopropanecarboxylic acid ethyl ester (0.48 g, 2.16 mmol) in DMF (10 mL) is treated with cesium carbonate (0.77 g, 2.4 mmol). The reaction is heated to 50° C. and stirred overnight. The reaction is cooled and quenched with 1N aqueous hydrochloric acid. The solution is partitioned in ethyl ether and water. The organic is separated, washed with brine, and dried over sodium sulfate. The organic is filtered and the solvent is removed to afford the crude product. The crude is purified by silica gel column chromatography using 9/1 Hexanes/EtOAc to elute two products. The solvent is removed to afford isomer 1 (0.33 g, 31%) and isomer 2 (0.345 g, 32%) of the desired product. ¹H NMR (400 MHz, CDCl₃); MS (ES⁺) m/z mass calcd for C₃₀H₃₃ClO₄ 492, found 493 (M+1, 100%).

### Step C

### 2-{4-[4-(4-Chloro-2-phenoxy-phenyl)-3-methyl-butoxy]-2-methyl-phenyl}-cyclopropanecarboxylic acid

A solution of 2-{4-[4-(4-chloro-2-phenoxy-phenyl)-3-methyl-butoxy]-2-methyl-phenyl}-cyclopropanecarboxylic acid ethyl ester (0.330 g, 0.7 mmol, Isomer 1) in ethanol (10 mL) is treated with 5N aqueous sodium hydroxide (1.3 mL). The reaction is heated to reflux and stirred for 3 hours. The reaction is cooled and the pH adjusted to pH=4 with 1N aqueous hydrochloric acid. The solution is extracted with EtOAc. The organic is washed with brine, dried over sodium sulfate and filtered. The solvent is removed to afford 0.26 g (84%) of title compound. ¹H NMR (400 MHz, CDCl₃); MS (ES⁺) m/z mass calcd for C₂₈H₂₉ClO₄ 464, found 465 (M+1, 100%).

### EXAMPLE 96

### 2-{4-[4-(4-Chloro-2-phenoxy-phenyl)-3-methyl-butoxy]-2-methyl-phenyl}-cyclopropanecarboxylic acid

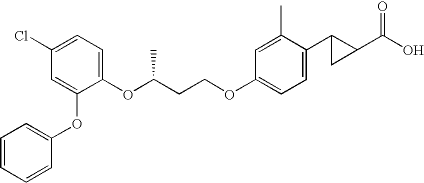

A solution of 2-{4-[4-(4-chloro-2-phenoxy-phenyl)-3-methyl-butoxy)-2-methyl-phenyl}-cyclopropanecarboxylic acid ethyl ester (0.345 g, 0.7 mmol, Isomer 2) in ethanol (15 mL) is treated with 5N aqueous sodium hydroxide (1.4 mL). The reaction is heated to reflux and stirred for 3 hours. The reaction is cooled and the pH adjusted to pH=4 with 1N aqueous hydrochloric acid. The solution is extracted with EtOAc. The organic is washed with brine, dried over sodium sulfate and filtered. The solvent is removed to afford 0.27 g (83%) of title compound. ¹H NMR (400 MHz, CDCl₃); MS (ES⁺) m/z mass calcd for C₂₈H₂₉ClO₄ 464, found 465 (M+1, 100%).

### EXAMPLE 97

### (S)-[4-(3-Methanesulfonyloxy-butoxy)-2-methyl-phenylsulfanyl]-acetic acid ethyl ester

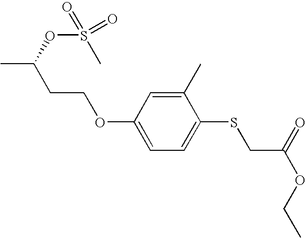

### Step A

### 4-Benzyloxy-2-methyl-1-methylsulfanyl-benzene

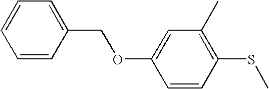

A mixture of 4-(methylthio)-m-cresol (10 g, 64.8 mmol) and 325 mesh K₂CO₃ (11.65 g, 84.3 mmol) in DMF (100 mL) is treated with benzyl bromide (12.22 g, 71.5 mmol) and stirred at room temperature for 17 hr under N₂. The mixture is filtered using Et₂O to rinse the solids, and the filtrate is acidified with 1 N HCl (65 mL). The filtrate is diluted with more Et₂O and then extracted twice with water and brine. The organic layer is dried (Na₂SO₄), and the solvent is removed in vacuo to afford 17.03 g (100%) of crude title compound that is carried on without purification. R_{f}=0.66 (1/1 hexanes/acetone). ¹H NMR (400 MHz, CDCl₃).

### Step B

### 1-Methanesulfinyl-4-benzyloxy-2-methyl-benzene

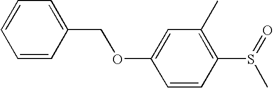

A 0° C. solution of crude 4-benzyloxy-2-methyl-1-methylsulfanyl-benzene (17.03 g, 64.8 mmol) in chloroform (300 mL) is treated with about 77% m-chloroperbenzoic acid (14.53 g, 64.8 mmol) in portions over 10 minutes. The reaction is stirred at 0° C. for 20 minutes and monitored closely by TLC (1/1 hexanes/acetone) until the crude material is gone (R_{f}=0.66) and the sulfoxide formed (R_{f}=0.27). The mixture is extracted with saturated NaHCO₃ and then saturated NaHSO₃. The organic layer is dried (MgSO₄), and the solvent is removed in vacuo to afford 18.32 g (100%) of crude title compound that is carried on without purification. R_{f}=0.27 (1/1 hexanes/acetone). ¹H NMR (400 MHz, CDCl₃); MS (ES⁺) m/z mass calcd for C₁₅H₁₆O₂S 260, found 261 (M+1, 100%).

### Step C

### (4-Benzyloxy-2-methyl-phenylsulfanyl)-acetic acid ethyl ester

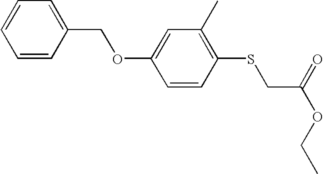

A solution of crude material from Step B (18.32 g, 64.8 mmol) in CH₂Cl₂ (250 mL) is treated with trifluoroacetic anhydride (27.2 g, 0.130 mol,) and the resultant purple solution is heated to reflux for 30 minutes under N₂. The reaction is cooled, and the solvent is removed in vacuo to give 25.21 g (100%) of an intermediate that is carried on without purification. R_{f}=0.66 (1/1 hexanes/acetone). The crude α-trifluoroacetoxy sulfide (25.21 g, assume 64.8 mmol) is combined with bromoEtOAc (59.02 g, 0.353 mol) in EtOH (230 mL) and purged with N₂ for 5 minutes. Potassium carbonate (325 mesh, 32.56 g, 0.236 mol) is added, and the mixture is stirred for 17 hours at rt under N₂. The mixture is filtered using Et₂O to rinse the solids, and the filtrate is acidified with 1 N HCl (100 mL). The filtrate is diluted with more Et₂O and extracted with water. The organic layer is dried (Na₂SO₄), and the solvent is removed in vacuo to afford crude product that is absorbed on silica gel and purified by flash chromatography using 10/1 hexanes/acetone to afford 6.45 g (35%) of the title compound. R_{f}=0.43 (2/1 hexanes/acetone). ¹H NMR (400 MHz, CDCl₃); MS (ES⁺) m/z mass calcd for C₁₈H₂₀O₃S 316, found 317 (M+1, 100%).

### Step D

### (4-Hydroxy-2-methyl-phenylsulfanyl)-acetic acid ethyl ester

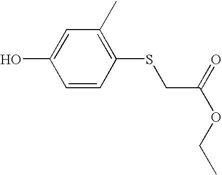

A solution (−78° C.) of material obtained in Step C (6.44 g, 20.4 mmol) and dimethylethylsilane (17.96 g, 0.203 mol) in CH₂Cl₂ (150 mL) is treated dropwise with a 1 M solution of TiCl₄ in CH₂Cl₂ (20.4 mL, 20.4 mmol). The mixture is warmed to 0° C. and then rt for 3 hours. The reaction is quenched with water and extracted with EtOAc. The organic layer is dried (Na₂SO₄), and the solvent is removed in vacuo to afford crude product that is absorbed on silica gel and purified by flash chromatography using 98/2 CH₂Cl₂/ACN to afford 2.96 g (64%) of the title compound. R_{f}=0.28 (2/1 hexanes/acetone). ¹H NMR (400 MHz, CDCl₃); MS (ES⁻) m/z mass calcd for C₁₁H₁₄O₃S 226, found 325 (M−1, 100%).

### Step E

### (S)-Toluene-4-sulfonic acid 3-hydroxy-butyl ester

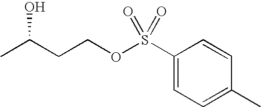

A solution of (S)-(+)-1,3-butanediol (9.5 g, 0.105 mol) and Et₃N (12.8 g, 0.126 mol) in CH₂Cl₂ (200 mL) is treated with dibutyltin oxide (0.52 g, 2.08 mmol) and then p-toluenesulfonyl chloride (20.09 g, 0.105 mol) is added as a solid in portions over 30 minutes at rt. The resultant mixture is stirred at rt for 17 hours under N₂. The reaction is quenched with 1 N HCl (50 mL), diluted with water and extracted with EtOAc. The organic layer is dried (Na₂SO₄), and the solvent is removed in vacuo to afford crude product that is absorbed on silica gel and purified by flash chromatography using 98/2 CH₂Cl₂/ACN (to elute the unreacted p-toluenesulfonyl chloride) and then 2/1 hexanes/acetone to afford 18.67 g (73%) the title compound. R_{f}=0.23, R_{f} bis-tosylate=0.53 (98/2 CH₂Cl₂/ACN).

### Step F

### (S)-[4-(3-Hydroxy-butoxy)-2-methyl-phenylsulfanyl]-acetic acid ethyl ester

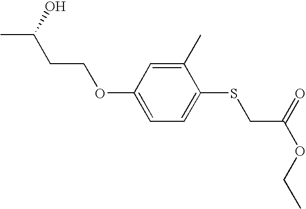

A mixture of (4-hydroxy-2-methyl-phenylsulfanyl)-acetic acid ethyl ester (2.96 g, 13.1 mmol), (S)-toluene-4-sulfonic acid 3-hydroxy-butyl ester (3.83 g, 15.7 mmol) and cesium carbonate (5.54 g, 0.169 mol) in dry DMF (55 mL) is heated to 5° C. for 17 hours under N₂. The reaction is cooled, quenched with 1 N HCl (40 mL), diluted with Et₂O and extracted with water. The organic layer is dried (Na₂SO₄), and the solvent is removed in vacuo to afford crude product that is absorbed on silica gel and purified by flash chromatography using a gradient of 6/1 to 2/1 hexanes/EtOAc to afford 2.30 g (59%) of the title compound. R_{f}=0.28 (1/1 hexanes/EtOAc). ¹H NMR (400 MHz, CDCl₃); MS (ES⁺) m/z mass calcd for C₁₅H₂₂O₄S 298, found 321 (+Na, 100%).

### Step G

### (S)-[4-(3-Methanesulfonyloxy-butoxy)-2-methyl-phenylsulfanyl]-acetic acid ethyl ester

A 0° C. solution of (S)-[4-(3-hydroxy-butoxy)-2-methyl-phenylsulfanyl]-acetic acid ethyl ester (2.29 g, 7.67 mmol) and Et₃N (1.94 g, 19.2 mmol) in CH₂Cl₂ (40 mL) is treated dropwise with MsCl (1.32 g, 11.5 mmol) and stirred at 0° C. for 2 hours under N₂. The reaction is quenched with 1 N HCl (23 mL), diluted with CH₂Cl₂ and then extracted with water. The organic layer is dried (Na₂SO₄), and the solvent is removed in vacuo to afford 3.20 g (100%) of the title compound. R_{f}=0.37 (1/1 hexanes/EtOAc). ¹H NMR (400 MHz, CDCl₃); MS (ES⁺) m/z mass calcd for C₁₆H₂₄O₆S₂ 376, found 377 (M+1, 100%).

### EXAMPLE 98

### (R)-{4-[3-(4-Ethyl-2-phenoxy-phenoxy)-butoxy]-2-methyl-phenylsulfanyl}-acetic acid

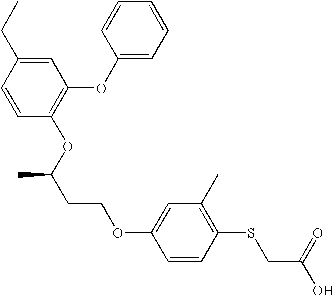

### Step A

### 4-Ethyl-1-methoxy-2-phenoxy-benzene

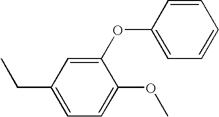

A mixture of 2-bromo-4-ethyl-1-methoxy-benzene (0.60 g, 2.79 mmol), phenol (0.525 g, 5.57 mmol), cesium carbonate (1.82 g, 5.58 mmol), copper (I) chloride (0.138 g, 1.39 mmol) and 2,2,6,6-tetramethyl-3,5-heptanedione (0.13 g, 0.706 mmol) in dry 1-methyl-2-pyrrolidinone (5 mL) is heated to 120° C. for 17 hours under N₂. The reaction is cooled, quenched with 1 N HCl (20 mL), diluted with Et₂O and extracted with water. The organic layer is dried (Na₂SO₄), and the solvent is removed in vacuo to afford crude product that is absorbed on silica gel and purified by flash chromatography using 9/1 hexanes/EtOAc to afford 0.604 g (95%) of the title compound. R_{f}=0.46 (4/1 hexanes/EtOAc). ¹H NMR (400 MHz, CDCl₃); MS (ES⁺) m/z mass calcd for C₁₅H₂₂O₄S 298, found 321 (M+Na, 100%).

### Step B

### 4-Ethyl-2-phenoxy-phenol

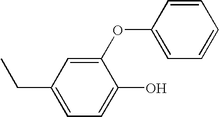

A 40° C. solution of 4-ethyl-1-methoxy-2-phenoxy-benzene (0.60 g, 2.62 mmol) in dry CH₂Cl₂ (5 mL) is treated dropwise with borontribromide (1.96 g, 7.83 mmol) and then warmed to 0° C. and stirred for 30 minutes under N₂. The reaction is diluted with Et₂O and quenched with water. The organic layer is dried (Na₂SO₄), and the solvent is removed in vacuo to afford crude product that is absorbed on silica gel and purified by flash chromatography using 2/1 hexanes/acetone to afford 0.448 g (80%) 4-ethyl-2-phenoxy-phenol. R_{f}=0.44 (2/1 hexanes/acetone). ¹H NMR (400 MHz, CDCl₃); MS (ES⁻) m/z mass calcd for C₁₄H₁₄O₄ 214, found 213 (M−1, 100%).

### Step C

### (R)-({4-[3-(4-Ethyl-2-phenoxy-phenoxy)-butoxy]-2-methyl-phenylsulfanyl}-acetic acid ethyl ester

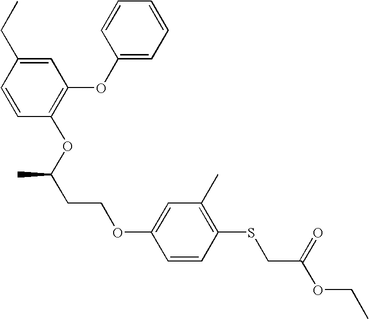

A mixture of 4-ethyl-2-phenoxy-phenol (0.141 g, 0.658 mmol), (s)-[4-(3-methanesulfonyloxy-butoxy)-2-methyl-phenylsulfanyl]-acetic acid ethyl ester (0.297 g, 0.789 mmol) (Example 97, Step G) and Cs₂CO₃ (0.279 g, 0.856 mmol) in dry DMF (10 mL) is heated to 60° C. and stirred for 17 hours under N₂. The mixture is cooled and acidified with 1 N HCl (20 mL). The mixture is diluted with water and extracted with Et₂O. The organic layer is dried (Na₂SO₄) and the solvent is removed in vacuo to afford crude product that is absorbed on silica gel and purified by column chromatography using 9/1 hexanes/EtOAc to afford 0.230 g (71%) of the title compound. R_{f}=0.30 (4/1 hexanes/EtOAc). ¹H NMR (400 MHz, CDCl₃); MS (ES⁺) m/z mass calcd for C₂₉H₃₄O₅S 494, found 495 (M+1,100%).

### Step D

### (R)-{4-[3-(4-Ethyl-2-phenoxy-phenoxy)-butoxy]-2-methyl-phenylsulfanyl}-acetic acid

A solution of (R)-({4-[3-(4-ethyl-2-phenoxy-phenoxy)-butoxy]-2-methyl-phenylsulfanyl}-acetic acid ethyl ester (0.230, 0.465 mmol) in ethanol (6 mL) is treated with 5 N NaOH (1 mL) and stirred at room temperature until saponification complete. The solvent removed in vacuo to afford a residue that is acidified with 1 N HCl. The mixture is diluted with water and extracted with EtOAc. The organic layer is dried (Na₂SO₄), and the solvent is removed in vacuo to afford 0.206 g (95%) of the title compound. ¹H NMR (400 MHz, CDCl₃); HRMS (ES⁺) m/z exact mass calcd for C₂₇H₃₀O₅S 466, found 467 (M+1, 100%).

### EXAMPLE 99

### (R)-{4-[3-(2-Benzoyl-4-methyl-phenoxy)-butoxy]-2-methyl-phenylsulfanyl}-acetic acid

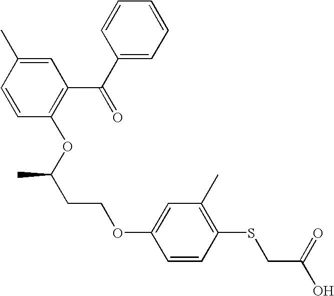

### Step A

### (R)-{4-[3-(2-Benzoyl-4-methyl-phenoxy)-butoxy]-2-methyl-phenylsulfanyl}-acetic acid ethyl ester

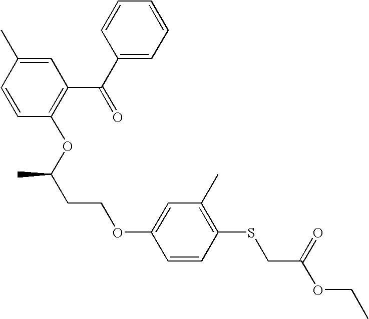

A mixture of (2-hydroxy-5-methyl-phenyl)-phenyl-methanone (0.189 g, 0.891 mmol), (S)-[4-(3-methanesulfonyloxy-butoxy)-2-methyl-phenylsulfanyl]-acetic acid ethyl ester (0.402 g, 1.07 mmol) (Example 97, Step G) and Cs₂CO₃ (0.377 g, 1.16 mmol) in dry DMF (15 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 9/1 hexanes/EtOAc to afford 0.326 g (74%) of the title compound. R_{f}=0.53 (98/2 CH₂Cl₂/ACN). ¹H NMR (400 MHz, CDCl₃); MS (ES⁺) m/z mass calcd for C₂₉H₃₂O₅S 492, found 493 (M+1, 100%).

### Step B

### (R)-{4-[3-(2-Benzoyl-4-methyl-phenoxy)-butoxy]-2-methyl-phenylsulfanyl}-acetic acid

A solution of (R)-{4-[3-(2-benzoyl-4-methyl-phenoxy)-butoxy]-2-methyl-phenylsulfanyl}-acetic acid ethyl ester (0.326, 0.662 mmol) in ethanol (10 mL) is treated with 5 N NaOH (2 mL) and stirred at rt until saponification complete. The solvent is removed in vacuo to afford a residue that is acidified with 1 N HCl. The mixture is diluted with water and extracted with EtOAc. The organic layer is dried (Na₂SO₄) and the solvent is removed in vacuo to afford 0.321 g (100%) of the title compound. ¹H NMR (400 MHz, CDCl₃); MS (ES⁺) m/z mass calcd for C₂₇H₂₈O₅S 464, found 465 (M+1, 100%).

### EXAMPLE 100

### (R)-{4-[3-(2-Benzoyl-4-trifluoromethoxy-phenoxy)-butoxy]-2-methyl-phenylsulfanyl}-acetic acid

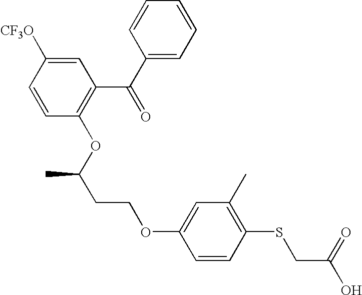

### Step A

### (R)-{4-[3-(2-Benzoyl-4-trifluoromethoxy-phenoxy)-butoxy]-2-methyl-phenylsulfanyl}-acetic acid ethyl ester

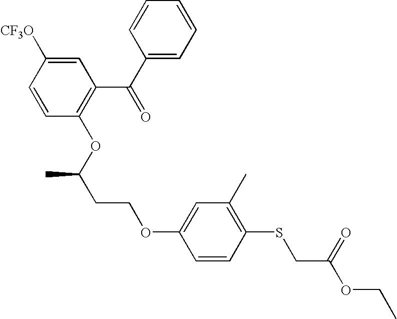

A mixture of (2-hydroxy-5-trifluoromethoxy-phenyl)-phenyl-methanone (0.286 g, 1.01 mmol), (s)-[4-(3-methanesulfonyloxy-butoxy)-2-methyl-phenylsulfanyl]-acetic acid ethyl ester (0.460 g, 1.22 mmol) (Example 97, Step G) and Cs₂CO₃ (0.40 g, 1.23 mmol) in dry DMF (25 mL) is heated to 50° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 6/1 hexanes/EtOAc to afford 0.291 g (51%) of the title compound. R_{f}=0.51 (1/1 hexanes/EtOAc). ¹H NMR (400 MHz, CDCl₃); MS (ES⁺) m/z mass calcd for C₂₉H₂₉O₆SF₃ 562, found 563 (M+1, 100%).

### Step B

### (R)-{4-[3-(2-Benzoyl-4-trifluoromethoxy-phenoxy)-butoxy]-2-methyl-phenylsulfanyl}-acetic acid

A solution of (R)-{4-[3-(2-benzoyl-4-methyl-phenoxy)-butoxy]-2-methyl-phenylsulfanyl}-acetic acid ethyl ester (0.291, 0.517 mmol) in ethanol (10 mL) is treated with 5 N NaOH (1 mL) and stirred at room temperature until saponification complete. The solvent is removed in vacuo to afford a residue that is acidified with 1N HCl. The mixture is diluted with water and extracted with EtOAc. The organic layer is dried (Na₂SO₄), and the solvent is removed in vacuo to afford 0.280 g (100%) of the title compound. ¹H NMR (400 MHz, CDCl₃); HRMS (ES⁺) m/z exact mass calcd for C₂₇H₂₆O₆SF₃ 535.1402, found 535.1396.

### EXAMPLE 101

### {4-[3-(2-Benzoyl-4-ethyl-phenoxy)-hexyloxy]-2-methyl-phenylsulfanyl}-acetic acid

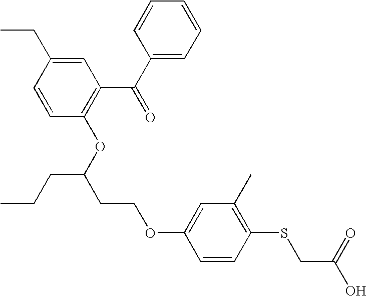

### Step A

### (5-Ethyl-2-methoxy-phenyl)-phenyl-methanone

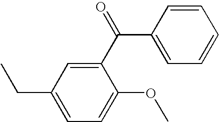

A 0° C. solution of 4-ethylanisole (10.0 g, 73.4 mmol) in dry CH₂Cl₂ (100 mL) is treated portion wise with aluminum chloride (11.7 g, 87.7 mmol). The 0° C. reaction mixture is then treated dropwise with benzoyl chloride (11.38 g, 81.0 mmol) and the reaction is stirred at 0° C. for 1 hour under N₂. The reaction is poured into ice water and extracted with CH₂Cl₂. The organic layer is dried (Na₂SO₄), and the solvent is removed in vacuo to afford crude product that is absorbed on silica gel and purified by flash chromatography using 9/1 hexanes/EtOAc to afford 14.72 g (83%) of the title compound. R_{f}=0.34 (4/1 hexanes/EtOAc). ¹H NMR (400 MHz, CDCl₃); MS (ES⁺) m/z mass calcd for C₁₆H₁₆O₂ 240, found 241 (M+1, 100%).

### Step B

### (5-Ethyl-2-methoxy-phenyl)-phenyl-methanone

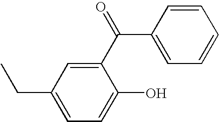

A mixture of (5-ethyl-2-methoxy-phenyl)-phenyl-methanone 2120203 (10.0 g, 41.6 mmol) and pyridine hydrochloride (48.1 g, 0.416 mol) is heated to 200° C. in an oil bath stirred for 30 minutes under N₂. The reaction is cooled diluted with Et₂O and washed twice with 1 N HCl and brine. The organic layer is dried (Na₂SO₄), and the solvent is removed in vacuo to afford 8.90 g (95%) of the title compound. R_{f}=0.55 (2/1 hexanes/EtOAc). ¹H NMR (400 MHz, CDCl₃); MS (ES) m/z mass calcd for C₁₅H₁₄O₂ 226, found 225 (M−1, 100%).

### Step C

### 3-Bromo-hexan-1-ol

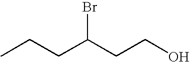

A −78° C. solution of ethyl β-bromocaproate (5.0 g, 22.4 mmol) in dry THF (50 mL) is treated dropwise with a 1 M solution of diisobutylaluminum hydride in cyclohexane (47 mL, 47.0 mmol). The mixture is stirred for 15 minutes at −78° C. and then warmed to 0° C. and stirred for 45 minutes under N₂. The reaction is slowly quenched with 1 N HCl (100 mL) and then diluted with water and extracted with Et₂O. The organic layer is dried (Na₂SO₄) and the solvent is removed in vacuo to afford 4.04 g (99%) of crude 3-bromo-hexan-1-ol that is utilized without purification.

### Step D

### {5-Ethyl-2-[1-(2-hydroxy-ethyl)-butoxy]-phenyl}-phenyl-methanone

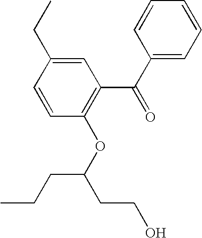

A mixture of (5-ethyl-2-methoxy-phenyl)-phenyl-methanone (1.00 g, 4.42 mmol), 3-bromo-hexan-1-ol (2.00 g, 11.0 mmol) and Cs₂CO₃ (4.32 g, 13.3 mmol) in dry DMF (20 mL) is heated to 50° C. and stirred for 17 hours under N₂. The reaction is cooled, filtered, and the filtrate is acidified with 1 N HCl (20 mL). The filtrate is diluted with water and extracted with Et₂O. The organic layer is dried (Na₂SO₄), and the solvent is removed in vacuo to afford crude product that is absorbed on silica gel and purified by column chromatography using a gradient of 6/1 then 3/1 hexanes/acetone to afford 1.03 g (71%) of the title compound. R_{f}=0.24 (2/1 hexanes/acetone). ¹H NMR (400 MHz, CDCl₃); MS (ES⁺) m/z mass calcd for C₂₁H₂₆O₃ 326, found 327 (M+1, 100%).

### Step E

Methanesulfonic acid 3-(2-benzoyl-4-ethyl-phenoxy)-hexyl ester

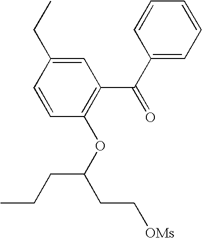

A 0° C. solution of {5-ethyl-2-[1-(2-hydroxy-ethyl)-butoxy]-phenyl}-phenyl-methanone (1.03 g, 3.16 mmol) and TEA (0.64 g, 6.32 mmol) in CH₂Cl₂ (25 mL) is treated with MsCl (0.592 g, 5.17 mmol), and the reaction is stirred for 1 hour at 0° C. under N₂. The reaction is quenched with 1 N HCl (7 mL) and diluted with more CH₂Cl₂ and extracted with water. The organic layer is dried (MgSO₄), and the solvent is removed in vacuo to afford 1.30 g (100%) of the title compound that is utilized without purification. ¹H NMR (400 MHz, CDCl₃).

### Step F

### {4-[3-(2-Benzoyl-4-ethyl-phenoxy)-hexyloxy]-2-methyl-phenylsulfanyl}-acetic acid

A mixture of (4-hydroxy-2-methyl-phenylsulfanyl)-acetic acid ethyl ester (0.081 g, 0.358 mmol), methanesulfonic acid 3-(2-benzoyl-4-ethyl-phenoxy)-hexyl ester (0.145 g, 0.359 mmol) and Cs₂CO₃ (0.140 g, 0.430 mmol) in dry DMF (7 mL) is heated to 50° C. and stirred for 17 hours under N₂. The reaction is treated with 5 N NaOH (2 mL) and cooled to room temperature and stirred 2 hours. The mixture is acidified with 1 N HCl (25 mL), diluted with water and extracted with Et₂O. The organic layer is dried (Na₂SO₄) and the solvent is removed in vacuo to afford 0.387 g of crude acid that is purified by preparative HPLC to afford 0.060 g (33%) of the title compound. ¹H NMR (400 MHz, CDCl₃); MS (ES⁺) m/z mass calcd for C₃₀H₃₄O₅S 506, found 507 (M+1, 100%).

### EXAMPLE 102

### 3-{4-[3-(2-Benzoyl-4-ethyl-phenoxy)-hexyloxy]-2-methyl-phenyl}-propionic acid

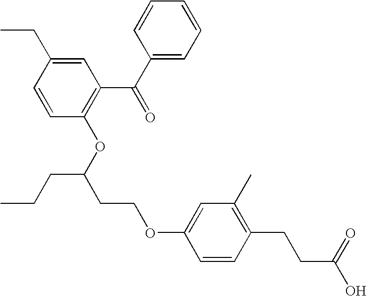

The title compound is prepared by following the procedure described in Example 101, Step F by utilizing 3-(4-hydroxy-2-methyl-phenyl)-propionic acid methyl ester to afford 0.314 g (57%). ¹H NMR (400 MHz, CDCl₃); HRMS (ES⁺) m/z exact mass calcd for C₃₁H₃₇O₅ 489.2641, found 489.2618.

### EXAMPLE 103

### {4-[3-(2-Benzoyl-4-ethyl-phenoxy)-hexyloxy]-2-methyl-phenoxy}-acetic acid

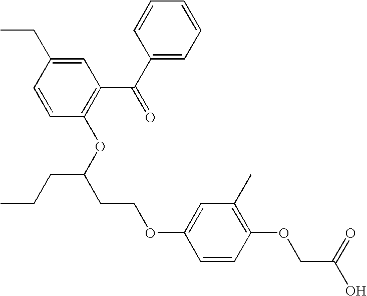

The title compound is prepared by following the procedure described in Example 101, Step F by utilizing (4-hydroxy-2-methyl-phenoxy)-acetic acid methyl ester to afford 0.062 g (54%). ¹H NMR (400 MHz, CDCl₃); MS (ES⁺) m/z mass calcd for C₃₀H₃₄O₆ 490, found 491.

### EXAMPLE 104

### 3-{4-[3-(2-Benzoyl-4-ethyl-phenoxy)-hexylsulfanyl]-2-methyl-phenyl}-propionic acid

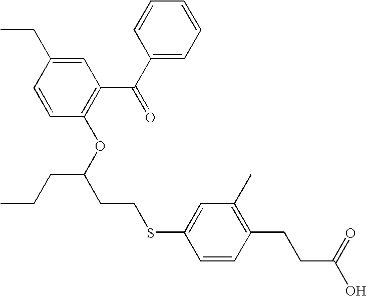

The title compound is prepared by following the procedure described in Example 101, Step F by utilizing 3-(4-mercapto-2-methyl-phenyl)-propionic acid methyl ester to afford 0.069 g (38%). ¹H NMR (400 MHz, CDCl₃); MS (ES⁺) m/z mass calcd for C₃₁H₃₆O₄S 504, found 505.

### EXAMPLE 105

### {4-[3-(2-Benzoyl-4-ethyl-phenoxy)-hexylsulfanyl]-2-methyl-phenoxy}-acetic acid

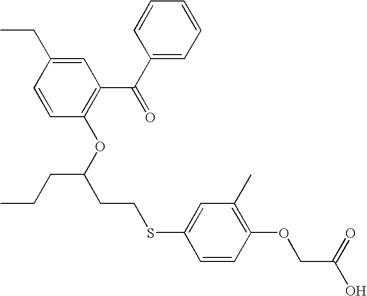

The title compound is prepared by following the procedure described in Example 101, Step F by utilizing (4-mercapto-2-methyl-phenoxy)-acetic acid ethyl ester to afford 0.069 g (38%). ¹H NMR (400 MHz, CDCl₃); MS (ES⁺) m/z mass calcd for C₃₀H₃₄O₅S 506, found 507.

### EXAMPLE 106

### (R)-3-{4-[3-(4-Ethyl-2-phenoxy-phenoxy)-1-methyl-propoxy]-2-methyl-phenyl}-propionic acid

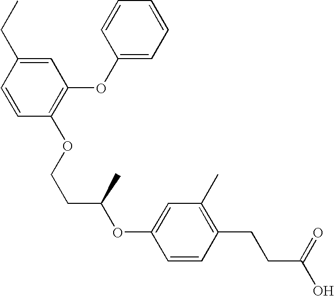

### Step A

### (s)-[5-Ethyl-2-(3-hydroxy-butoxy)-phenyl]-phenyl-methanone

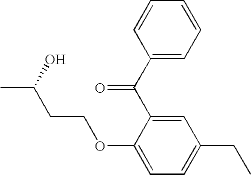

A mixture of (5-ethyl-2-methoxy-phenyl)-phenyl-methanone (2.96 g, 13.1 mmol), (S)-toluene-4-sulfonic acid 3-hydroxy-butyl ester (1.19 g, 4.87 mmol) and cesium carbonate (1.73 g, 5.31 mol) in dry DMF (25 mL) is heated to 55° C. for 17 hours under N₂. The reaction is cooled, quenched with 1 N HCl (20 mL), diluted with Et₂O and extracted with water. The organic layer is dried (Na₂SO₄), and the solvent is removed in vacuo to afford crude product that is absorbed on silica gel and purified by flash chromatography using a gradient of 4/1 to 2/1 hexanes/EtOAc to afford 0.860 g (65%) of the title compound. R_{f}=0.29 (1/1 hexanes/EtOAc).¹H NMR (400 MHz, CDCl₃); MS (ES⁺) m/z mass calcd for C₁₉H₂₂O₃ 298, found 321 (M+Na, 100%).

### Step B

### (S)-Methanesulfonic acid 3-(2-benzoyl-4-ethyl-phenoxy)-1-methyl-propyl ester

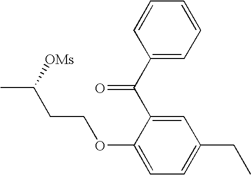

A 0° C. solution of (S)-[5-ethyl-2-(3-hydroxy-butoxy)-phenyl]-phenyl-methanone (0.86 g, 2.88 mmol) and Et₃N (0.73 g, 7.21 mmol) in CH₂Cl₂ (15 mL) is treated dropwise with MsCl (0.488 g, 4.26 mmol) and stirred at 0° C. for 2 hours under N₂. The reaction is quenched with 1 N HCl (9 mL), diluted with CH₂Cl₂ and then extracted with water. The organic layer is dried (Na₂SO₄) and the solvent is removed in vacuo to afford 1.12 g (100%) of the title compound. R_{f}=0.38 (1/1 hexanes/EtOAc). ¹H NMR (400 MHz, CDCl₃); MS (ES⁺) m/z mass calcd for C₂₀H₂₄O₅S 376, found 377 (M+1, 100%).

### Step C

### (R)-3-{4-[3-(4-Ethyl-2-phenoxy-phenoxy)-1-methyl-propoxy]-2-methyl-phenyl}-propionic acid methyl ester

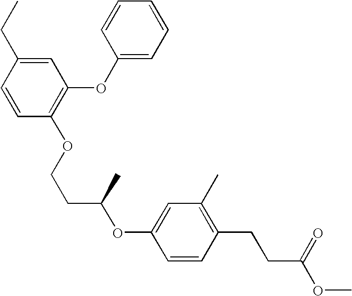

A mixture of 3-(4-hydroxy-2-methyl-phenyl)-propionic acid methyl ester (0.281 g, 1.45 mmol), (S)-methanesulfonic acid 3-(2-benzoyl-4-ethyl-phenoxy)-1-methyl-propyl ester (0.600 g, 1.59 mmol) and Cs₂CO₃ (0.570 g, 1.75 mmol) in dry DMF (15 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 98/2 98/2 CH₂Cl₂/ACN to afford 0.411 g (60%) of the title compound. R_{f}=0.46 (98/2 CH₂Cl₂/ACN). ¹H NMR (400 MHz, CDCl₃); MS (ES⁺) m/z mass calcd for C₃₀H₃₄O₅ 474, found 475 (M+1, 100%).

### Step D

### (R)-3-{4-[3-(4-Ethyl-2-phenoxy-phenoxy)-1-methyl-propoxy]-2-methyl-phenyl}-propionic acid

A solution of (R)-3-{4-[3-(4-ethyl-2-phenoxy-phenoxy)-1-methyl-propoxy]-2-methyl-phenyl}-propionic acid methyl ester (0.411, 0.866 mmol) in methanol (12 mL) is treated with 5 N NaOH (3 mL) and stirred at rt until saponification complete. The solvent removed in vacuo to afford a residue that is acidified with 1 N HCl. The mixture is diluted with water and extracted with EtOAc. The organic layer is dried (Na₂SO₄, and the solvent is removed in vacuo to afford 0.418 g (100%) of the title compound. ¹H NMR (400 MHz, CDCl₃); HRMS (ES⁺) m/z exact mass calcd for C₂₉H₃₃O₅ 461.2328, found 461.2335.

### EXAMPLE 107

### (R)-3-{4-[3-(4-Ethyl-2-phenoxy-phenoxy)-butoxy]-2-methyl-phenyl}-propionic acid

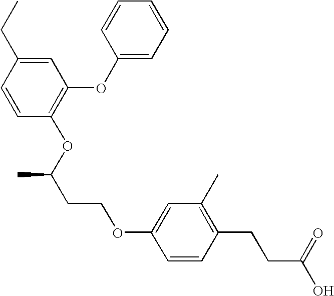

### Step A

### (S)-3-[4-(3-Hydroxy-butoxy)-2-methyl-phenyl]-propionic acid methyl ester

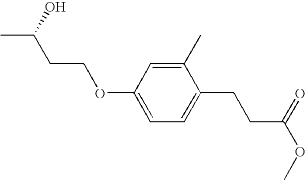

A mixture of 3-(4-hydroxy-2-methyl-phenyl)-propionic acid methyl ester (3.47 g, 17.9 mmol), (S)-toluene-4-sulfonic acid 3-hydroxy-butyl ester (5.23 g, 21.4 mmol) and cesium carbonate (7.57 g, 23.2 mol) in dry DMF (70 mL) is heated to 50° C. for 17 hours under N₂. The reaction is cooled, filtered, and the filtrate is quenched with 1 N HCl (50 mL). The filtrate is then diluted with Et₂O and extracted with water. The organic layer is dried (Na₂SO₄), and the solvent is removed in vacuo to afford crude product that is absorbed on silica gel and purified by flash chromatography using 4/1 hexanes/EtOAc to afford 3.07 g (65%) of the title compound. R_{f}=0.33 (1/1 hexanes/EtOAc). ¹H NMR (400 MHz, CDCl₃); MS (ES⁺) m/z mass calcd for C₁₅H₂₂O₄ 266, found 367 (M+1, 100%).

### Step B

### (S)-3-[4-(3-Methanesulfonyloxy-butoxy)-2-methyl-phenyl]-propionic acid methyl ester

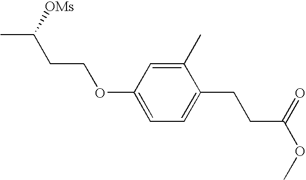

A 0° C. solution of (s)-3-[4-(3-hydroxy-butoxy)-2-methyl-phenyl]-propionic acid methyl ester (3.07 g, 11.5 mmol) and Et₃N (2.92 g, 28.9 mmol) in CH₂Cl₂ (50 mL) is treated dropwise with MsCl (1.98 g, 17.3 mmol) and stirred at 0° C. for 1.5 hours under N₂. The reaction is quenched with 1 N HCl (30 mL), diluted with CH₂Cl₂ and then extracted with water. The organic layer is dried (Na₂SO₄) and the solvent is removed in vacuo to afford 4.17 g (100%) of the title compound. R_{f}=0.45 (1/1 hexanes/EtOAc). ¹H NMR (400 MHz, CDCl₃); MS (ES⁺) m/z mass calcd for C₁₆H₂₄O₆S₂ 344, found 362 (M+NH₄, 100%).

### Step C

### (R)-3-{4-[3-(4-Ethyl-2-phenoxy-phenoxy)-butoxy]-2-methyl-phenyl}-propionic acid methyl ester

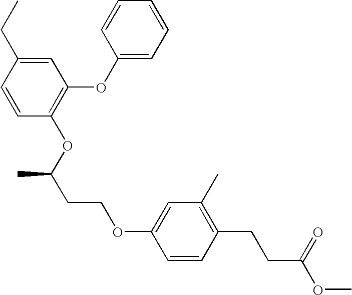

A mixture of 4-ethyl-2-phenoxy-phenol (0.214 g, 0.726 mmol), (S)-3-[4-(3-methanesulfonyloxy-butoxy)-2-methyl-phenyl]-propionic acid methyl ester (0.300 g, 0.871 mmol) and Cs₂CO₃ (0.308 g, 0.945 mmol) in dry DMF (10 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford crude product that is absorbed on silica gel and purified by column chromatography using 9/1 hexanes/EtOAc to afford 0.216 g (64%) of the title compound. R_{f}=0.30 (4/1 hexanes/EtOAc). ¹H NMR (400 MHz, CDCl₃); MS (ES⁺) m/z mass calcd for C₂₉H₃₄O₅ 462, found 463 (M+1, 100%).

### Step D

### (R)-3-{4-[3-(4-Ethyl-2-phenoxy-phenoxy)-butoxy]-2-methyl-phenyl}-propionic acid

A solution of (R)-3-{4-[3-(4-ethyl-2-phenoxy-phenoxy)-butoxy]-2-methyl-phenyl}-propionic acid methyl ester 0.216, 0.467 mmol) in methanol (6 mL) is treated with 5 N NaOH (1 mL) and stirred at rt until saponification complete. The solvent removed in vacuo to afford a residue that is acidified with 1 N HCl. The mixture is diluted with water and extracted with EtOAc. The organic layer is dried (Na₂SO₄), and the solvent is removed in vacuo to afford 0.195 g (93%) of the title compound. ¹H NMR (400 MHz, CDCl₃); HRMS (ES⁺) m/z exact mass calcd for C₂₈H₃₂O₅ 448, found 449 (M+1, 100%).

### EXAMPLE 108

### (R)-3-(4-{3-[4-Ethyl-2-(1-phenyl-vinyl)-phenoxy]-butoxy}-2-methyl-phenyl)-propionic acid

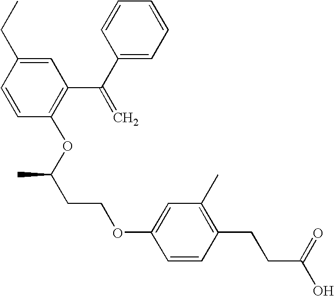

### Step A

### 4-Ethyl-2-(1-phenyl-vinyl)-benzene

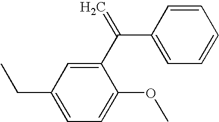

A 0° C. solution of (5-ethyl-2-methoxy-phenyl)-phenyl-methanone (1.00 g, 4.16 mmol) in anhydrous Et₂O (10 mL) is treated dropwise with a 3 M solution of methylmagnesium bromide in Et₂O (2.10 g, 6.30 mmol) and stirred at 0° C. for 1 hour under N₂. The reaction is acidified with 1 N HCl, diluted with Et₂O and then extracted with water. The organic layer is dried (Na₂SO₄), and the solvent is removed in vacuo to afford 1.06 g (100%) of crude 1-(5-ethyl-2-methoxy-phenyl)-1-phenyl-ethanol. R_{f}=0.43 (2/1 hexanes/acetone).

The alcohol intermediate is dissolved dissolved in toluene (20 mL), treated with p-toluenesulfonic acid monohydrate (0.160 g, 0.841 mmol) and is heated to reflux to remove the water generated in the reaction. Upon completion, the mixture is cooled and diluted with EtOAc, which is then extracted with water and saturated NaHCO₃. The organic layer is dried (Na₂SO₄) and the solvent is removed in vacuo to afford 1.47 g crude product that is purified by column chromatography using 5/1 hexanes acetone to afford 1.17 g (100%) of the title compound. R_{f}=0.65 (2/1 hexanes/acetone). ¹H NMR (400 MHz, CDCl₃); MS (ES⁺) m/z mass calcd for C₁₇H₁₈O 338, found 239 (M+1, 100%).

### Step B

### 4-Ethyl-2-(1-phenyl-vinyl)-phenol

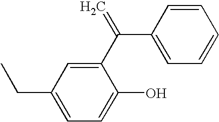

A mixture of 4-ethyl-2-(1-phenyl-vinyl)-benzene (0.638 g, 2.68 mmol) and pyridine hydrochloride (6.20 g, 53.7 mol) is heated to 200° C. in an oil bath and stirred for 5 hours under N₂. The reaction is cooled, diluted with Et₂O and washed twice with 1 N HCl and brine. The organic layer is dried (Na₂SO₄), and the solvent is removed in vacuo to afford crude product that is absorbed on silica gel and purified by column chromatography using 3/1 hexanes acetone to afford 0.378 g (63%) of the title compound. R_{f}=0.33 (2/1 hexanes/acetone). ¹H NMR (400 MHz, CDCl₃); MS (ES⁺) m/z mass calcd for C₁₆H₁₆O 224, found 225 (M+1, 100%).

### Step C

### (R)-3-(4-{3-[4-Ethyl-2-(1-phenyl-vinyl)-phenoxy]-butoxy}-2-methyl-phenyl)-propionic acid methyl ester

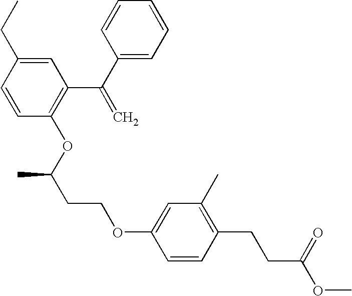

A mixture of 4-ethyl-2-(1-phenyl-vinyl)-phenol (0.188 g, 0.838 mmol), (S)-3-[4-(3-methanesulfonyloxy-butoxy)-2-methyl-phenyl]-propionic acid methyl ester (0.346 g, 1.00 mmol) and Cs₂CO₃ (0.330 g, 1.01 mmol) in dry DMF (15 mL) is heated to 60° C. and stirred for 17 hours under N₂. The reaction is cooled and acidified with 1N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 6/1 hexanes/EtOAc to afford 0.155 g (39%) of the title compound. R_{f}=0.44 (2/1 hexanes/acetone). ¹H NMR (400 MHz, CDCl₃); MS (ES⁺) m/z mass calcd for C₃₁H₃₆O₄ 472, found 490 (M+NH₄, 100%).

### Step D

### (R)-3-(4-{3-[4-Ethyl-2-(1-phenyl-vinyl)-phenoxy]-butoxy}-2-methyl-phenyl)-propionic acid

A solution of (R)-3-(4-{3-[4-ethyl-2-(1-phenyl-vinyl)-phenoxy]-butoxy}-2-methyl-phenyl)-propionic acid methyl ester (0.150, 0.317 mmol) in methanol (6 mL) is treated with 5 N NaOH (1 mL) and stirred at room temperature until saponification complete. The solvent is removed in vacuo to afford a residue that is acidified with 1N HCl. The mixture is diluted with water and extracted with EtOAc. The organic layer is dried (Na₂SO₄), and the solvent is removed in vacuo to afford 0.118 g of crude acid that is purified by preparative HPLC to afford 0.028 g (19%) of the title compound. ¹H NMR (400 MHz, CDCl₃); HRMS (ES⁺) m/z exact mass calcd for C₂₉H₃₃O₅ 461.2328, found 461.2350.

### EXAMPLE 109

### (R)-3-(4-{3-[4-Ethyl-2-(1-methyl-1-phenyl-ethyl)-phenoxy]-butoxy}-2-methyl-phenyl)-propionic acid

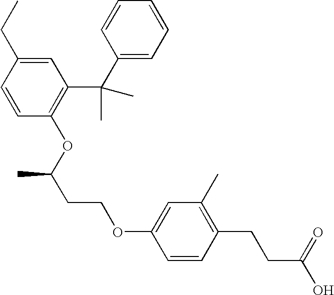

### Step A

### 4-Ethyl-2-(1-methyl-1-phenyl-ethyl)-benzene

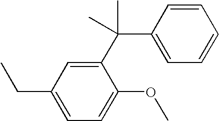

A 1M solution of titanium (TV) chloride (3.75 mL, 7.49 mmol) is cooled to −30° C. and treated dropwise with a 2 M solution dimethylzinc in toluene (3.75 g, 7.49 mmol). The mixture is stirred at −30° C. for 20 minutes under N₂. A solution of (5-ethyl-2-methoxy-phenyl)-phenyl-methanone (0.60 g, 2.50 mmol) in CH₂Cl₂ (4 mL) is added dropwise, and the reaction is stirred for 15 minutes at −30° C. and then warmed to rt and stirred for 1.5 hours. The mixture is slowly poured into a dry ice/methanol mixture, stirred and warmed to rt for 2 hours. The mixture is diluted with Et₂O and then extracted with water. The organic layer is dried (Na₂SO₄), and the solvent is removed in vacuo to afford crude product that is absorbed on silica gel and purified by column chromatography using 9/1 hexanes/EtOAc to afford 0.601 g (95%) of the title compound. R_{f}=0.60 (4/1 hexanes/EtOAc). ¹H NMR (400 MHz, CDCl₃).

### Step B

### 4-Ethyl-2-(1-methyl-1-phenyl-ethyl)-phenol

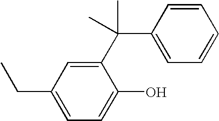

A −40° C. solution of 4-ethyl-2-(1-methyl-1-phenyl-ethyl)-benzene (0.600 g, 2.36 mmol) in CH₂Cl₂ (10 mL) is treated dropwise with borontribromide (1.78 g, 7.09 mmol) and then warmed to 0° C. and stirred for 1 hour under N₂. The reaction is diluted with Et₂O and quenched with water. The organic layer is dried (Na₂SO₄), and the solvent is removed in vacuo to afford crude product that is absorbed on silica gel and purified by column chromatography using 2/1 hexane/acetone to afford 0.545 g (96%) of the title compound. R_{f}=0.44 (2/1 hexane/acetone). ¹H NMR (400 MHz, CDCl₃); MS (ES⁻) m/z mass calcd for C₁₇H₂₀O 240, found 239 (M−1, 100%).

### Step C

### (R)-3-(4-{3-[4-Ethyl-2-(1-methyl-1-phenyl-ethyl)-phenoxy]-butoxy}-2-methyl-phenyl)-propionic acid methyl ester

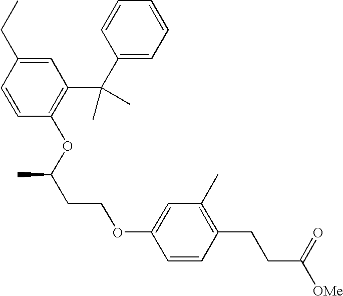

A mixture of 4-ethyl-2-(1-methyl-1-phenyl-ethyl)-phenol (0.100 g, 0.416 mmol), (S)-3-[4-(3-methanesulfonyloxy-butoxy)-2-methyl-phenyl]-propionic acid methyl ester (0.172 g, 0.499 mmol) and Cs₂CO₃ (0.176 g, 0.540 mmol) in dry DMF (8 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 7/1 hexanes/EtOAc to afford 0.097 g (48%) of the title compound. R_{f}=0.48 (1/1 hexanes/EtOAc). ¹H NMR (400 MHz, CDCl₃); MS (ES⁺) m/z mass calcd for C₃₂H₄₀O₄ 488, found 506 (M+NH₄, 100%).

### Step D

### (R)-3-(4-{3-[4-Ethyl-2-(1-methyl-1-phenyl-ethyl)-phenoxy]-butoxy}-2-methyl-phenyl)-propionic acid

A solution of (R)-3-(4-{3-[4-ethyl-2-(1-methyl-1-phenyl-ethyl)-phenoxy]-butoxy}-2-methyl-phenyl)-propionic acid methyl ester (0.097, 0.199 mmol) in methanol (10 mL) is treated with 5 N NaOH (2 mL) and stirred at rt until saponification is complete. The solvent is removed in vacuo to afford a residue that is acidified with 1N HCl. The mixture is diluted with water and extracted with EtOAc. The organic layer is dried (Na₂SO₄), and the solvent is removed in vacuo to afford 0.095 g of crude acid that is purified by preparative HPLC to afford 0.043 g (46%) of the title compound. ¹H NMR (400 MHz, CDCl₃); HRMS (ES⁺) m/z exact mass calcd for C₃₁H₄₂NO₄ (M+NH₄) 492.3114, found 492.3128.

### EXAMPLE 110

### (R)-3-{4-[3-(2-Benzoyl-4-methyl-phenoxy)-butoxy]-2-methyl-phenyl}-propionic acid

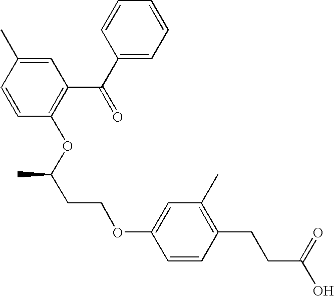

### Step A

### (R)-3-{4-[3-(2-Benzoyl-4-methyl-phenoxy)-butoxy]-2-methyl-phenyl}-propionic acid methyl ester

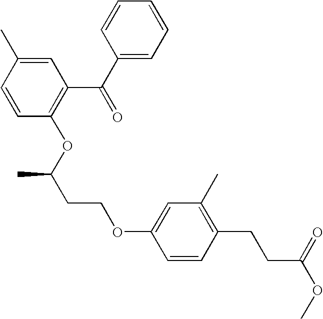

A mixture of (2-hydroxy-5-methyl-phenyl)-phenyl-methanone (0.200 g, 0.942 mmol), (S)-3-[4-(3-methanesulfonyloxy-butoxy)-2-methyl-phenyl]-propionic acid methyl ester (0.390 g, 1.13 mmol) and Cs₂CO₃ (0.400 g, 1.23 mmol) in dry DMF (12 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 7/1 hexanes/EtOAc to afford 0.332 g (76%) of the title compound. R_{f}=0.45 (1/1 hexanes/EtOAc). ¹H NMR (400 MHz, CDCl₃); MS (ES⁺) m/z mass calcd for C₂₉H₃₂O₅ 460, found 461 (M+1, 100%).

### Step B

### (R)-3-{4-[3-(2-Benzoyl-4-methyl-phenoxy)-butoxy]-2-methyl-phenyl}-propionic acid

A solution of (R)-3-{4-[3-(2-benzoyl-4-methyl-phenoxy)-butoxy]-2-methyl-phenyl}-propionic acid methyl ester (0.332, 0.721 mmol) in methanol (10 mL) is treated with 5 N NaOH (2 mL) and stirred at rt until saponification is completed. The solvent is removed in vacuo to afford a residue that is acidified with 1 N HCl. The mixture is diluted with water and extracted with EtOAc. The organic layer is dried (Na₂SO₄), and the solvent is removed in vacuo to afford 0.318 g (99%) of the title compound. ¹H NMR (400 MHz, CDCl₃); HRMS (ES⁺) m/z exact mass calcd for C₂₈H₃₁O₅ 447.2171, found 447.2174.

### EXAMPLE 111

### (R)-3-(4-{3-[4-Ethyl-2-(1-phenyl-ethyl)-phenoxy]-butoxy}-2-methyl-phenyl)-propionic acid

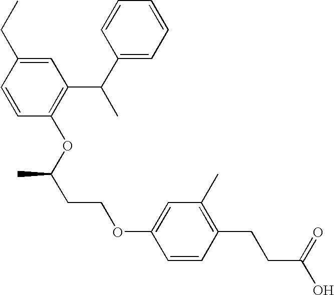

### Step A

### 4-Ethyl-2-(1-phenyl-ethyl)-benzene

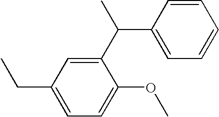

A mixture of 4-ethyl-2-(1-phenyl-vinyl)-benzene (1.00 g, 4.20 mmol) and 10% palladium on carbon in anhydrous ethanol (40 mL) is purged with N₂, purged with hydrogen and then stirred at rt under a hydrogen balloon for 7 hours. The reaction is filtered through hyflo, and the solvent is removed in vacuo to afford a residue that is dissolved in Et₂O and dried (Na₂SO₄). The organic layer is filtered, and the solvent is removed in vacuo to afford 0.952 g (95%) of the title compound. R_{f}=0.58 (4/1 hexanes/EtOAc). ¹H NMR (400 MHz, CDCl₃).

### Step B

### 4-Ethyl-2-(1-phenyl-ethyl)-phenol

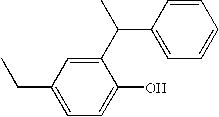

A −40° C. solution of 4-ethyl-2-(1-phenyl-ethyl)-benzene (0.950 g, 3.95 mmol) in CH₂Cl₂ (15 mL) is treated dropwise with borontribromide (2.97 g, 11.8 mmol) and then warmed to 0° C. and stirred for 1.5 hours under N₂. The reaction is diluted with Et₂O and quenched with water. The organic layer is dried (Na₂SO₄), and the solvent is removed in vacuo to afford crude product that is absorbed on silica gel and purified by column chromatography using 3/1 hexanes/acetone to afford 0.860 g (96%) of the title compound. R_{f}=0.59 (1/1 hexanes/EtOAc). ¹H NMR (400 MHz, CDCl₃); MS (ES⁻) m/z mass calcd for C₁₆H₁₈O 226, found 225 (M−1, 100%).

### Step C

### (R)-3-(4-{3-[4-Ethyl-2-(1-phenyl-ethyl)-phenoxy]-butoxy}-2-methyl-phenyl)-propionic acid

A mixture of 4-ethyl-2-(1-phenyl-ethyl)-phenol (0.102 g, 0.451 mmol), (S)-3-[4-(3-methanesulfonyloxy-butoxy)-2-methyl-phenyl]-propionic acid methyl ester (0.170 g, 0.494 mmol) and Cs₂CO₃ (0.175 g, 0.537 mmol) in dry DMF, (7 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford crude ester that is dissolved in methanol (6 mL) is treated with 5 N NaOH (2 mL) and stirred at rt until saponification complete. The solvent is removed in vacuo to afford a residue that is acidified with 1 N HCl. The mixture is diluted with water and extracted with EtOAc. The organic layer is dried (Na₂SO₄), and the solvent is removed in vacuo to afford 0.587 g of crude acid that is purified by preparative HPLC to afford 0.063 g (30%) of the title compound. ¹H NMR (400 MHz, CDCl₃); HRMS (ES⁺) m/z exact mass calcd for C₃₀H₃₇O₄ 461.2692, found 461.2705.

### EXAMPLE 112

### (R)-3-(4-{3-[4-Ethyl-2-(pyridine-2-carbonyl)-phenoxy]-butoxy}-2-methyl-phenyl)-propionic acid

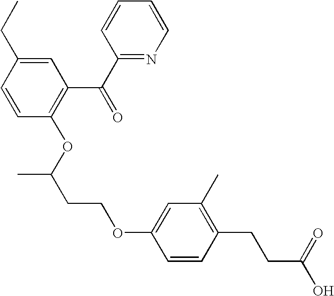

### Step A

### Pyridine-2-carboxylic acid methoxy-methyl-amide

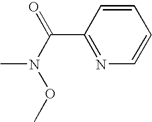

A 0° C. mixture of picolinoyl chloride hydrochloride (2.00 g, 11.2 mmol) and N,O-dimethylhydroxylamine (1.32 g, 13.5 mmol) in CH₂Cl₂ (50 mL) is treated dropwise with TEA (3.41 g, 33.7 mmol), and the reaction is stirred at 0° C. for 15 minutes is then warmed to rt and stirred for 1 hour under N₂. The mixture is diluted with CH₂Cl₂ and washed with water. The organic layer is dried (Na₂SO₄), and the solvent is removed in vacuo to afford 1.44 g (77%) the title compound that is utilized without purification. R_{f}=0.10 (1/1 hexanes/EtOAc). ¹H NMR (400 MHz, CDCl₃); MS (ES⁺) m/z mass calcd for C₈H₁₀O₂N₂ 166, found 167 (M+1, 100%).

### Step B

### (5-Ethyl-2-methoxy-phenyl)-pyridin-2-yl-methanone

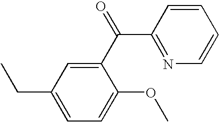

A −10° C. solution of N,N,N′,N′-tetramethylethylenediamine (1.31 g, 11.3 mmol) is treated dropwise with a 1.6 M solution of n-butyllithium in hexanes (7.2 mL, 11.5 mmol), and the reaction is stirred at −10° C. under N₂. 4-Ethylanisole (1.08 g, 7.93 mmol) is then added dropwise, and the mixture is stirred at −10° C. under N₂. Pyridine-2-carboxylic acid methoxy-methyl-amide (1.47 g, 8.85 mmol) is added and the mixture is stirred at −10° C. for 40 minutes under N₂. The mixture is quenched with water and extracted with Et₂O. The organic layer is dried (Na₂SO₄), and the solvent is removed in vacuo to afford crude product that is absorbed on silica gel and purified by column chromatography using 7/1 hexanes/acetone to afford 0.132 g (6%) of the title compound. R_{f}=0.38 (1/1 hexanes/EtOAc). ¹H NMR (400 MHz, CDCl₃); MS (ES⁺) m/z mass calcd for C₁₅H₁₅O₂N 241, found 242 (M+1, 100%).

### Step C

### (5-Ethyl-2-hydroxy-phenyl)-pyridin-2-yl-methanone

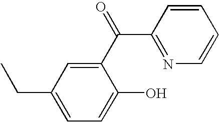

A −40° C. solution of (5-ethyl-2-methoxy-phenyl)-pyridin-2-yl-methanone (0.132 g, 0.547 mmol) in CH₂Cl₂ (5 mL) is treated dropwise with borontribromide (0.424 g, 1.69 mmol) and warmed to 0° C. and then rt, which is then stirred under N₂ until the reaction is completed. The reaction is cooled to 0° C., diluted with Et₂O, quenched with water and the pH is adjusted to pH=7 with 1 N NaOH. The organic layer is dried (Na₂SO₄), and the solvent is removed in vacuo to afford 0.102 g (82%) of the title compound that is utilized without purification. R_{f}=0.55 (1/1 hexanes/EtOAc). ¹H NMR (400 MHz, CDCl₃); MS (ES) m/z mass calcd for C₁₄H₁₃NO₂ 227, found 228 (M+1, 100%).

### Step D

### (R)-3-(4-{3-[4-Ethyl-2-(pyridine-2-carbonyl)-phenoxy]-butoxy}-2-methyl-phenyl)-propionic acid methyl ester

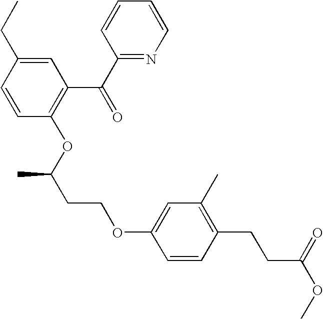

A mixture of 5-ethyl-2-hydroxy-phenyl)-pyridin-2-yl-methanone (0.102 g, 0.449 mmol), (S)-3-[4-(3-methanesulfonyloxy-butoxy)-2-methyl-phenyl]-propionic acid methyl ester (0.170 g, 0.494 mmol) and Cs₂CO₃ (0.175 g, 0.537 mmol) in dry DMF (7 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford crude product that is absorbed on silica gel and purified by column chromatography using 98/2 CH₂Cl₂/ACN to afford 0.054 g (25%) of the title compound. R_{f}=0.15 (98/2 CH₂Cl₂/ACN). ¹H NMR (400 MHz, CDCl₃); MS (ES⁺) m/z mass calcd for C₂₉H₃₃NO₅ 475, found 476 (M+1, 100%).

### Step E

### (R)-3-(4-{3-[4-Ethyl-2-(pyridine-2-carbonyl)-phenoxy]-butoxy}-2-methyl-phenyl)-propionic acid

A solution of (R)-3-(4-{3-[4-ethyl-2-(pyridine-2-carbonyl)-phenoxy]-butoxy}-2-methyl-phenyl)-propionic acid methyl ester (0.054, 0.114 mmol) in methanol (6 mL) is treated with 5 N NaOH (1 mL) and stirred at rt until saponification is completed. The solvent is removed in vacuo to afford a residue that is neutralized to pH=7 with 1 N HCl. The mixture is diluted with water and extracted with EtOAc. The organic layer is dried (Na₂SO₄), and the solvent is removed in vacuo to afford 0.052 g (100%) of the title compound. ¹H NMR (400 MHz, CDCl₃); HRMS (ES⁺) m/z exact mass calcd for C₂₈H₃₂NO₅ 462.2280, found 462.2281.

### EXAMPLE 113

### 3-(2-Methyl-4-{3-[2-(thiophene-2-carbonyl)-4-trifluoromethoxy-phenoxy]-butoxy}-phenyl)-propionic acid

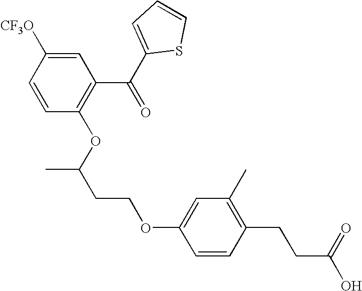

### Step A

### Thiophene-2-carboxylic acid methoxy-methyl-amide

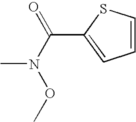

The procedure from Example 112, Step A is utilized with thiophene-2-carbonyl chloride to afford 4.09 g (92%) of the title compound. R_{f}=0.28 (2/1 hexanes/acetone). ¹H NMR (400 MHz, CDCl₃); MS (ES⁺) m/z mass calcd for C₇H₉O₂Ns 171, found 172 (M+1, 100%).

### Step B

### (2-Methoxy-5-trifluoromethoxy-phenyl)-thiophen-2-yl-methanone

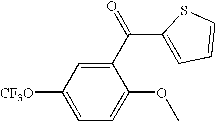

The procedure from Example 112, Step B is utilized with thiophene-2-carboxylic acid methoxy-methyl-amide to afford 1.52 g (24%) of the title compound. R_{f}=0.51 (98/2 CH₂Cl₂/ACN). ¹H NMR (400 MHz, CDCl₃).

### Step C

### (2-Hydroxy-5-trifluoromethoxy-phenyl)-thiophen-2-yl-methanone

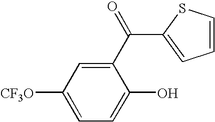

The procedure from Example 112, Step C is utilized with (2-methoxy-5-trifluoromethoxy-phenyl)-thiophen-2-yl-methanone to afford 1.18 g (82%) of the title compound after column purification with 98/2 CH₂Cl₂/ACN. R_{f}=0.76 (98/2 CH₂Cl₂/ACN). ¹H NMR (400 MHz, CDCl₃); MS (ES⁻) m/z mass calcd for C₁₂H₇O₃F₃S 288, found 287 (M−1, 100%).

### Step D

### 3-(2-Methyl-4-{3-[2-(thiophene-2-carbonyl)-4-trifluoromethoxy-phenoxy]-butoxy}-phenyl)-propionic acid methyl ester

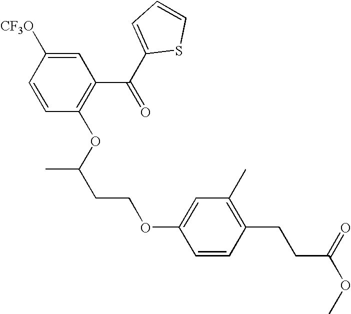

A mixture of (2-hydroxy-5-trifluoromethoxy-phenyl)-thiophen-2-yl-methanone (0.100 g, 0.347 mmol), 3-[4-(3-methanesulfonyloxy-butoxy)-2-methyl-phenyl]-propionic acid methyl ester (0.144 g, 0.418 mmol) and Cs₂CO₃ (0.136 g, 0.417 mmol) in dry DMF (10 mL) is heated to 50° C. and stirred for 17 hours under N₂. The reaction is cooled and acidified with 1 N HCl (25 mL). The mixture is diluted with water and extracted with Et₂O. The organic layer is dried (Na₂SO₄), and the solvent is removed in vacuo to afford crude product that is absorbed on silica gel and purified by column chromatography using 10/1 hexanes/acetone to afford 0.085 g (45%) of the title compound. R_{f}=0.26 (2/1 hexanes/acetone). ¹H NMR (400 MHz, CDCl₃); MS (ES⁺) m/z mass calcd for C₂₇H₂₇SO₆F₃ 536, found 537 (M+1, 100%).

### Step E

### 3-(2-Methyl-4-{3-[2-(thiophene-2-carbonyl)-4-trifluoromethoxy-phenoxy]-butoxy}-phenyl)-propionic acid

A solution of 3-(2-methyl-4-{3-[2-(thiophene-2-carbonyl)-4-trifluoromethoxy-phenoxy]-butoxy}-phenyl)-propionic acid methyl ester (0.085, 0.158 mmol) in methanol (6 mL) is treated with 5 N NaOH (1 mL) and stirred at rt until saponification complete. The solvent is removed in vacuo to afford a residue that is neutralized to pH=7 with 1 N HCl. The mixture is diluted with water and extracted with EtOAc. The organic layer is dried (Na₂SO₄) and the solvent is removed in vacuo to afford 0.075 g (90%) of the title compound. ¹H NMR (400 MHz, CDCl₃); MS (ES⁺) m/z mass calcd for C₂₆H₂₅SO₆F₃ 522, found 523 (M+1, 100%).

### EXAMPLE 114

### 3-(4-{3-[4-Ethyl-2-(thiophene-2-carbonyl)-phenoxy]-butoxy}-2-methyl-phenyl)-propionic acid

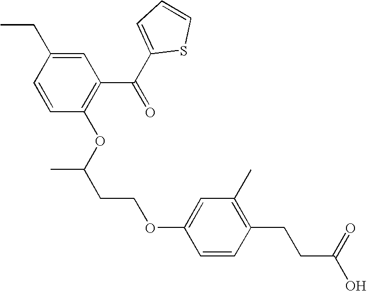

### Step A

### (5-Ethyl-2-methoxy-phenyl)-thiophen-2-yl-methanone

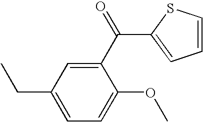

The procedure from Example 101, Step A is utilized with thiophene-2-carbonyl chloride to afford 8.61 g (95%) of the title compound. ¹H NMR (400 MHz, CDCl₃); MS (ES⁺) m/z mass calcd for C₁₄H₁₄O₂S 246, found 247 (M+1, 100%).

### Step B

### (5-Ethyl-2-hydroxy-phenyl)-thiophen-2-yl-methanone

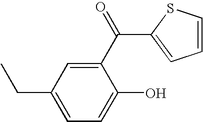

The procedure from Example 101, Step B is utilized with (5-ethyl-2-methoxy-phenyl)-thiophen-2-yl-methanone to afford 7.34 g (91%) of the title compound. ¹H NMR (400 MHz, CDCl₃); MS (ES⁻) m/z mass calcd for C₁₃H₁₂O₂S 232, found 231 (M−1, 100%).

### Step C

### 3-(4-{3-[4-Ethyl-2-(thiophene-2-carbonyl)-phenoxy]-butoxy}-2-methyl-phenyl)-propionic acid methyl ester

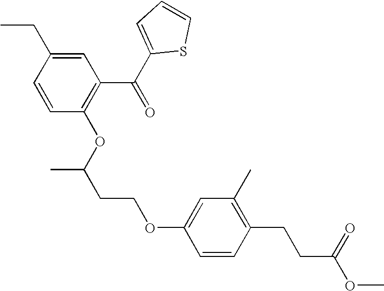

A mixture of (5-ethyl-2-hydroxy-phenyl)-thiophen-2-yl-methanone (0.111 g, 0.478 mmol), 3-[4-(3-methanesulfonyloxy-butoxy)-2-methyl-phenyl]-propionic acid methyl ester (0.206 g, 0.598 mmol) and Cs₂CO₃ (0.187 g, 0.574 mmol) in dry DMF (8 mL) is heated to 50° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 10/1 hexanes/acetone to afford 0.165 g (72%) of the title compound. R_{f}=0.22 (2/1 hexanes/acetone). ¹H NMR (400 MHz, CDCl₃); MS (ES⁺) m/z mass calcd for C₂₈H₃₂O₅S 480, found 481 (M+1, 100%).

### Step D

### 3-(4-{3-[4-Ethyl-2-(thiophene-2-carbonyl)-phenoxy]-butoxy}-2-methyl-phenyl)-propionic acid

A solution of 3-(4-{3-[4-ethyl-2-(thiophene-2-carbonyl)-phenoxy]-butoxy}-2-methyl-phenyl)-propionic acid methyl ester (0.165, 0.343 mmol) in methanol (6 mL) is treated with 5 N NaOH (1 mL) and stirred at rt until saponification is completed. The solvent is removed in vacuo to afford a residue that is acidified with 1 N HCl. The mixture is diluted with water and extracted with EtOAc. The organic layer is dried (Na₂SO₄) and the solvent is removed in vacuo to afford 0.170 g (100%) of the title compound. ¹H NMR (400 MHz, CDCl₃); HRMS (ES⁺) m/z exact mass calcd for C₂₇H₃₁O₅S 467.1892, found 467.1887.

### EXAMPLE 115

### 3-(4-{3-[2-(Benzo[b]thiophene-2-carbonyl)-4-ethyl-phenoxy]-butoxy}-2-methyl-phenyl)-propionic acid

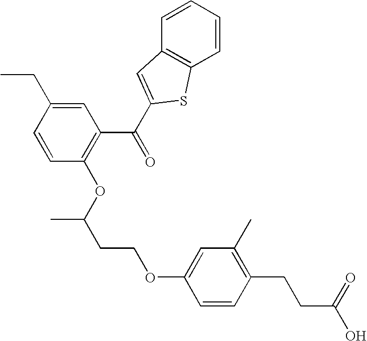

### Step A

### Benzo[b]thiophen-2-yl-(5-ethyl-2-methoxy-phenyl)-methanone

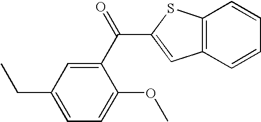

The procedure from Example 101, Step A is utilized with benzo[b]thiophene-2-carbonyl chloride to afford 1.29 g (91%) of the title compound. ¹H NMR (400 MHz, CDCl₃); MS (ES⁺) m/z mass calcd for C₁₈H₁₆O₂S 296, found 297 (M+1, 100%).

### Step B

### Benzo[b]thiophen-2-yl-(5-ethyl-2-hydroxy-phenyl)-methanone

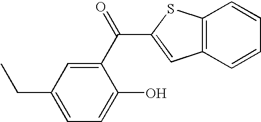

The procedure from Example 101, Step B is utilized with benzo[b]thiophen-2-yl-(5-ethyl-2-methoxy-phenyl)-methanone to afford 0.91 g (74%) of the title compound. ¹H NMR (400 MHz, CDCl₃); MS (ES⁻) m/z mass calcd for C₁₇H₁₄O₂S 282, found 281 (M−1, 100%).

### Step C

### 3-(4-{3-[2-(Benzo[b]thiophene-2-carbonyl)-4-ethyl-phenoxy]-butoxy}-2-methyl-phenyl)-propionic acid

A mixture of benzo[b]thiophen-2-yl-(5-ethyl-2-hydroxy-phenyl)-methanone (0.082 g, 0.290 mmol), 3-[4-(3-methanesulfonyloxy-butoxy)-2-methyl-phenyl]-propionic acid methyl ester (0.105 g, 0.305 mmol) and Cs₂CO₃ (0.119 g, 0.365 mmol) in dry DMF (7 mL) is heated to 50° C. and stirred for 17 hours under N₂. The reaction is treated with 5 N NaOH (2 mL), and then cooled and stirred at rt for 3 hours. The mixture is acidified with 1 N HCl, diluted with water, and then extracted with Et₂O. The organic layer is dried (Na₂SO₄), and the solvent is removed in vacuo to afford 0.427 g crude acid that is purified by preparative HPLC to give 0.024 g (16%) of the title compound. ¹H NMR (400 MHz, CDCl₃); MS (ES⁺) m/z mass calcd for C₃₁H₃₂O₅S 516, found 517.

### EXAMPLE 116

### 3-(4-{3-[4-Ethyl-2-(naphthalene-1-carbonyl)-phenoxy]-butoxy}-2-methyl-phenyl)-propionic acid

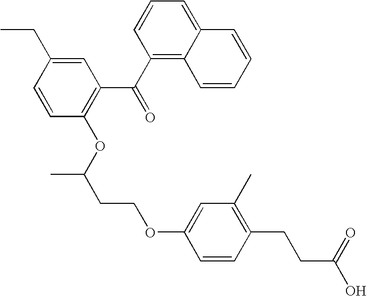

### Step A

### (5-Ethyl-2-methoxy-phenyl)-naphthalen-1-yl-methanone

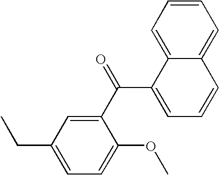

The procedure from Example 101, Step A is utilized with naphthalene-1-carbonyl chloride to afford 10.42 g (98%) of the title compound. ¹H NMR (400 MHz, CDCl₃); MS (ES⁺) m/z mass calcd for C₂₀H₁₈O₂ 290, found 291 (M+1, 100%).

### Step B

### (5-Ethyl-2-hydroxy-phenyl)-naphthalen-1-yl-methanone

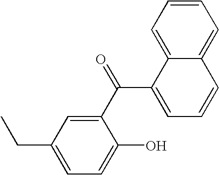

The procedure from Example 101, Step B is utilized with (5-ethyl-2-methoxy-phenyl)-naphthalen-1-yl-methanone to afford 9.63 g (97%) of the title compound. ¹H NMR (400 MHz, CDCl₃); MS (ES⁻) m/z mass calcd for C₁₉H₁₆O₂ 276, found 275 (M−1,100%).

### Step C

### 3-(4-{3-[4-Ethyl-2-(naphthalene-1-carbonyl)-phenoxy]-butoxy}-2-methyl-phenyl)-propionic acid

The procedure from Example 115, Step C is utilized with (5-ethyl-2-hydroxy-phenyl)-naphthalen-1-yl-methanone to afford 0.056 g (47%) of the title compound. ¹H NMR (400 MHz, CDCl₃); MS (ES⁻) m/z mass calcd for C₃₃H₃₄O₅ 510, found 509 (M−1).

### EXAMPLE 117

### 3-(4-{3-[4-Ethyl-2-(1-phenyl-vinyl)-phenoxy]-butoxy}-2-methyl-phenyl)-propionic acid

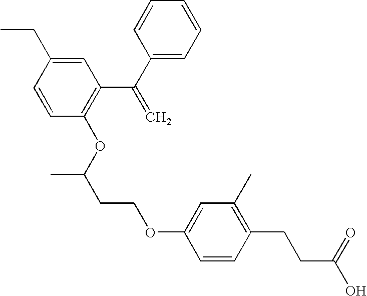

The title compound is prepared according to the procedure described in Example 115, Step C by utilizing 4-ethyl-2-(1-phenyl-vinyl)-phenol to afford 0.009 g (6%). ¹H NMR (400 MHz, CDCl₃); MS (ES⁻) m/z mass calcd for C₃₀H₃₄O₄ 458, found 457(M−1).

### EXAMPLE 118

### 3-{4-[3-(2-Benzoyl-phenoxy)-butoxy]-2-methyl-phenyl}-propionic acid

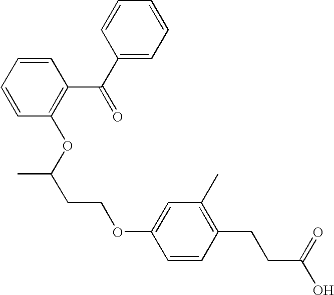

The title compound is prepared according to the procedure described in Example 115, Step C by utilizing (2-hydroxy-phenyl)-phenyl-methanone to afford 0.034 g (35%). ¹H NMR (400 MHz, CDCl₃); HRMS (ES⁺) m/z exact mass calcd for C₂₇H₂₉O₅ 433.2015, found 433.2003.

### EXAMPLE 119

### 3-{4-[3-(2-Benzoyl-4-methyl-phenoxy)-butoxy]-2-methyl-phenyl}-propionic acid

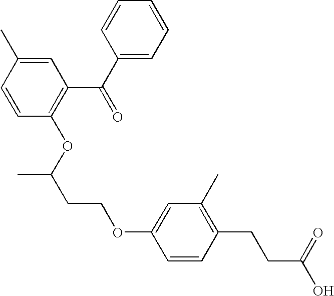

The title compound is prepared according to the procedure described in Example 115, Step C by utilizing (2-hydroxy-5-methyl-phenyl)-phenyl-methanone to afford 0.025 g (30%). ¹H NMR (400 MHz, CDCl₃); HRMS (ES⁺) m/z exact mass calcd for C₂₈H₃]O₅ 447.2171, found 447.2150.

### EXAMPLE 120

### (R)-3-{2-Methyl-4-[3-(quinolin-5-yloxy)-butoxy]-phenyl}-propionic acid hydrochloride

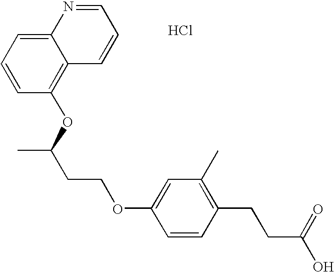

The title compound is prepared according to the procedure described in Example 115, Step C by utilizing (S)-3-[4-(3-methanesulfonyloxy-butoxy)-2-methyl-phenyl]-propionic acid methyl ester and quinolin-5-ol to afford 0.029 g (24%). ¹H NMR (400 MHz, CDCl₃); MS (ES⁺) m/z mass calcd for C₂₃H₂₅NO₄ 379, found 380 (M+1, 100%).

### EXAMPLE 121

### (R)-3-{2-Methyl-4-[3-(2-methyl-quinolin-8-yloxy)-butoxy]-phenyl}-propionic acid hydrochloride

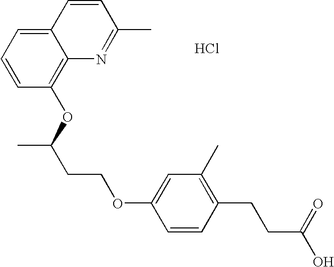

The title compound is prepared according to the procedure described in Example 115, Step C by utilizing (S)-3-[4-(3-methanesulfonyloxy-butoxy)-2-methyl-phenyl]-propionic acid methyl ester and 2-methyl-quinolin-8-ol to afford 0.007 g (6%). ¹H NMR (400 MHz, CDCl₃); MS (ES⁺) m/z mass calcd for C₂₄H₂₇NO₄ 393, found 394 (M+1, 100%).

### EXAMPLE 122

### (R)-3-{2-Methyl-4-[3-(quinolin-8-yloxy)-butoxy]-phenyl}-propionic acid hydrochloride

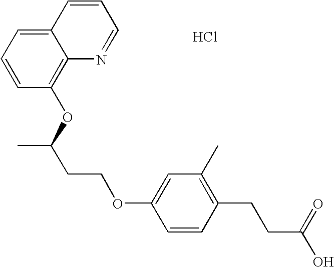

The title compound is prepared according to the procedure described in Example 115, Step C by utilizing (S)-3-[4-(3-methanesulfonyloxy-butoxy)-2-methyl-phenyl]-propionic acid methyl ester and quinolin-8-ol to afford 0.025 g (21%). ¹H NMR (400 MHz, CDCl₃); MS (ES⁺) m/z mass calcd for C₂₃H₂₅NO₄ 379, found 380 (M+1, 100%).

### EXAMPLE 123

### (R)-3-{4-[3-(Isoquinolin-5-yloxy)-butoxy]-2-methyl-phenyl}-propionic acid hydrochloride

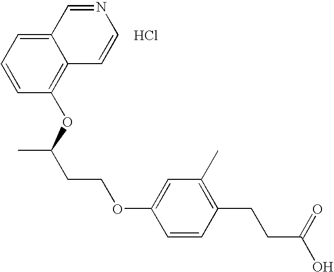

The title compound is prepared according to the procedure described in Example 115, Step C by utilizing (S)-3-[4-(3-methanesulfonyloxy-butoxy)-2-methyl-phenyl]-propionic acid methyl ester and isoquinolin-5-ol to afford 0.037 g (31%). ¹H NMR (400 MHz, CDCl₃); MS (ES⁺) m/z mass calcd for C₂₃H₂₅NO₄ 379, found 380 (M+1, 100%).

### EXAMPLE 124

### (R)-3-{4-[3-(5-Chloro-quinolin-8-yloxy)-butoxy]-2-methyl-phenyl}-propionic acid

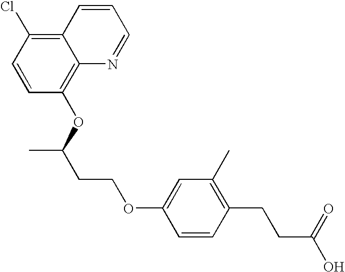

The title compound is prepared according to the procedure described in Example 115, Step C by utilizing (S)-3-[4-(3-methanesulfonyloxy-butoxy)-2-methyl-phenyl]-propionic acid methyl ester and 5-chloro-quinolin-8-ol to afford 0.088 g (49%). ¹H NMR (400 MHz, CDCl₃); MS (ES⁺) m/z mass calcd for C₂₃H₂₄NO₄Cl 413, found 414 and 415 (M+1 and M+3, 100%).

### EXAMPLE 125

### 3-{4-[3-(2-Benzyl-4-ethyl-phenoxy)-butoxy]-2-methyl-phenyl}-propionic acid

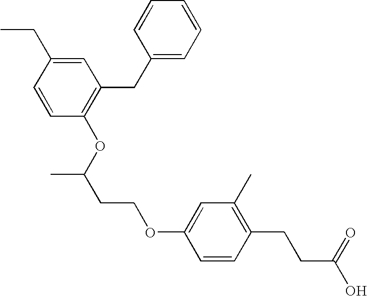

### Step A

### 2-Benzyl-4-ethyl-phenol

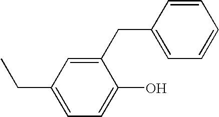

A mixture of (5-ethyl-2-methoxy-phenyl)-phenyl-methanone (1.0 g, 4.16 mmol) and triethylsilane (2.90 g, 24.9 mmol) is treated with TFA (10 mL), and the reaction is stirred at rt for 5 hours under N₂. The solvent is removed in vacuo to afford a residue that is diluted with EtOAc and extracted with water and saturated NaHCO₃. The organic layer is dried (Na₂SO₄), and the solvent is removed in vacuo to afford 1.27 g of an oil. The oil is dissolved in CH₂Cl₂ (15 mL), cooled to −40° C. and treated dropwise with borontribromide (6.36 g, 25.4 mmol), which then warmed to 0° C. and stirred for 1.5 hours under N₂. The reaction is diluted with Et₂O and quenched with water. The organic layer is dried (Na₂SO₄) and the solvent is removed in vacuo to afford crude product that is absorbed on silica gel and purified by column chromatography using 6/1 hexanes/acetone to give 0.657 g (74%) of the title compound. R_{f}=0.27 (2/1 hexanes/acetone). ¹H NMR (400 MHz, CDCl₃); MS (ES⁻) m/z mass calcd for C₁₅H₁₆O 212, found 211 (M−1, 100%).

### Step B

### 3-{4-[3-(2-Benzyl-4-ethyl-phenoxy)-butoxy]-2-methyl-phenyl}-propionic acid

The procedure from Example 115, Step C is utilized with 2-benzyl-4-ethyl-phenol to afford 0.037 g (34%) of the title compound. ¹H NMR (400 MHz, CDCl₃); HRMS (ES⁺) m/z exact mass calcd for C₂₉H₃₅O₄ 447.2535, found 447.2525.

### EXAMPLE 126

### 3-{4-[3-(2-Benzoyl-4-bromo-phenoxy)-butoxy]-2-methyl-phenyl}-propionic acid

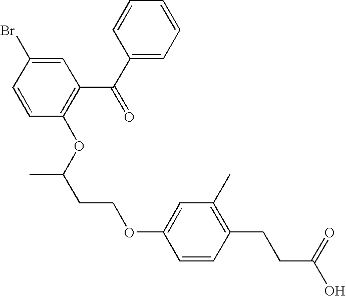

### Step A

### 3-{4-[3-(2-Benzoyl-4-bromo-phenoxy)-butoxy]-2-methyl-phenyl}-propionic acid methyl ester

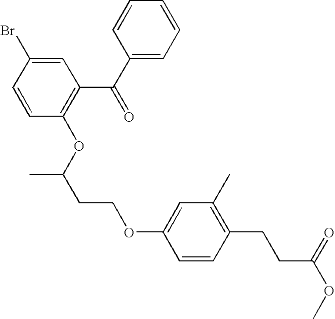

A mixture of (5-bromo-2-hydroxy-phenyl)-phenyl-methanone (0.285 g, 1.03 mmol), 3-[4-(3-methanesulfonyloxy-butoxy)-2-methyl-phenyl]-propionic acid methyl ester (0.393 g, 1.14 mmol) and Cs₂CO₃ (0.402 g, 1.23 mmol) in dry DMF (10 mL) is heated to 50° C. and stirred for 17 hours under N₂. The reaction is cooled and acidified with 1 N HCl (25 mL). The mixture is diluted with water and extracted with Et₂O. The organic layer is dried (Na₂SO₄) and the solvent is removed in vacuo to afford crude product that is absorbed on silica gel and purified by column chromatography using 10/1 hexanes/acetone to afford 0.357 g (66%) of the title compound. R_{f}=0.25 (2/1 hexanes/acetone). ¹H NMR (400 MHz, CDCl₃); MS (ES⁺) m/z mass calcd for C₂₈H₂₉O₅Br 524, found 525 and 527 (M+1 and M+3, 100%).

### Step B

### 3-{4-[3-(2-Benzoyl-4-bromo-phenoxy)-butoxy]-2-methyl-phenyl}-propionic acid

A solution of 3-{4-[3-(2-benzoyl-4-bromo-phenoxy)-butoxy]-2-methyl-phenyl}-propionic acid methyl ester (0.064, 0.122 mmol) in methanol (6 mL) is treated with 5 N NaOH (0.5 mL) and stirred at rt until saponification is completed. The solvent is removed in vacuo to afford a residue that is acidified with 1 N HCl. The mixture is diluted with water and extracted with EtOAc. The organic layer is dried (Na₂SO₄) and the solvent is removed in vacuo to afford 0.073 g (100%) of the title compound. ¹H NMR (400 MHz, CDCl₃); HRMS (ES⁺) m/z exact mass calcd for C₂₇H₂₇O₅BrNa 533.0940, found 533.0949.

### EXAMPLE 127

### 3-{4-[3-(2-Benzoyl-4-butyl-phenoxy)-butoxy]-2-methyl-phenyl}-propionic acid

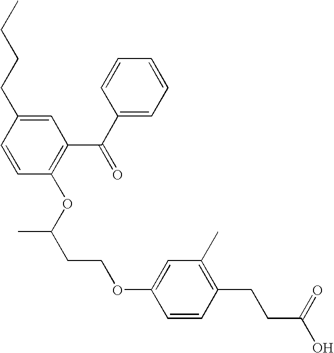

### Step A

### 3-{4-[3-(2-Benzoyl-4-butyl-phenoxy)-butoxy]-2-methyl-phenyl}-propionic acid methyl ester

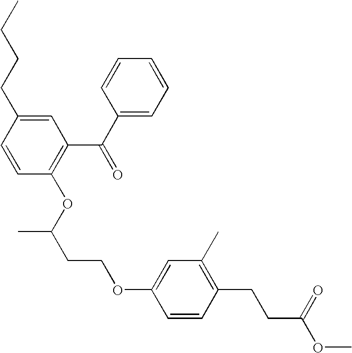

The compounds of 3-{4-[3-(2-benzoyl-4-bromo-phenoxy)-butoxy]-2-methyl-phenyl}-propionic acid methyl ester (0.128 g, 0.244 mmol) (Example 126, Step A), n-butylboronic acid (0.075 g, 0.736 mmol) and cesium fluoride (0.130 g, 0.856 mmol) are combined in 1,4-dioxane (6 mL) and purged with N₂. The reaction is treated with 1,1′-bis(diphenylphosphino)ferrocene palladium (II)chloride and CH₂Cl₂ complex (0.027 g, 0.037 mmol) and then heated in an oil bath at 80° C. for 10 hours under N₂. The reaction is cooled, and the solvent is removed in vacuo to afford crude product that is absorbed on silica gel and column purified using 10/1 hexanes/acetone to afford 0.066 g (54%) of the title compound. R_{f}=0.26 (2/1 hexanes/acetone). ¹H NMR (400 MHz, CDCl₃); MS (ES⁺) m/z mass calcd for C₃₂H₃₈O₅ 502, found 503 (M+1, 100%).

### Step B

### 3-{4-[3-(2-Benzoyl-4-butyl-phenoxy)-butoxy]-2-methyl-phenyl}-propionic acid

A solution of 3-{4-[3-(2-benzoyl-4-butyl-phenoxy)-butoxy]-2-methyl-phenyl}-propionic acid methyl ester (0.066, 0.131 mmol) in methanol (6 mL) is treated with 5 N NaOH (0.7 mL) and stirred at rt until saponification is completed. The solvent is removed in vacuo to afford a residue that is acidified with 1 N HCl. The mixture is diluted with water and extracted with EtOAc. The organic layer is dried (Na₂SO₄) and the solvent is removed in vacuo to afford 0.060 g (94%) of the title compound. ¹H NMR (400 MHz, CDCl₃); MS (ES⁺) m/z mass calcd for C₃₁H₃₆O₅ 488, found 489.

### EXAMPLE 128

### 3-{4-[3-(2-Benzoyl-4-propyl-phenoxy)-butoxy]-2-methyl-phenyl}-propionic acid

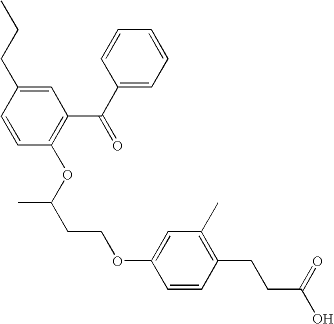

### Step A

### 3-{4-[3-(2-Benzoyl-4-propyl-phenoxy)-butoxy]-2-methyl-phenyl}-propionic acid methyl ester

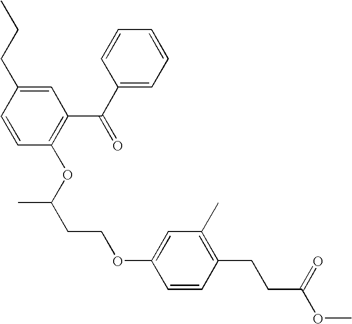

The procedure from Example 127, Step A is utilized with n-propylboronic acid to afford 0.055 g (54%) the title compound. R_{f}=0.34 (2/1 hexanes/acetone). ¹H NMR (400 MHz, CDCl₃); MS (ES⁺) m/z mass calcd for C₃₁H₃₆O₅ 488, found 485 (M+1, 100%).

### Step B

### 3-{4-[3-(2-Benzoyl-4-propyl-phenoxy)-butoxy]-2-methyl-phenyl}-propionic acid

The procedure from Example 127, Step B is utilized with 3-{4-[3-(2-benzoyl-4-propyl-phenoxy)-butoxy]-2-methyl-phenyl}-propionic acid methyl ester to afford 0.052 g (98%) of the title compound. ¹H NMR (400 MHz, CDCl₃); HRMS (ES⁺) m/z exact mass calcd for C₃₀H₃₅O₅ 475.2484, found 475.2485.

### EXAMPLE 129

### 3-{4-[4-(2-Benzoyl-4-ethyl-phenoxy)-1-methyl-butoxy]-2-methyl-phenyl}-propionic acid

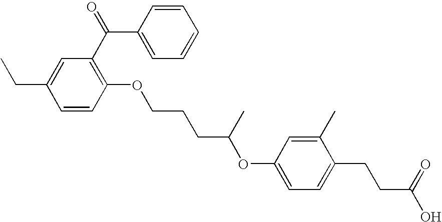

### Step A

### Toluene-4-sulfonic acid 4-hydroxy-pentyl ester

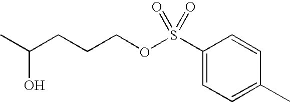

A solution of 1,4-pentanediol (4.60 g, 44.2 mmol) and Et₃N (5.36 g, 52.9 mmol) in CH₂Cl₂ (100 mL) is treated with dibutyltin oxide (0.22 g, 0.884 mmol), and then p-toluenesulfonyl chloride (8.42 g, 44.2 mmol) is added as a solid in portions over 30 minutes at rt. The resultant mixture is stirred at rt for 6 hours under N₂. The reaction is quenched with 1 N HCl (25 mL), diluted with water and extracted with CH₂Cl₂. The organic layer is dried (Na₂SO₄) and the solvent is removed in vacuo to afford crude product that is absorbed on silica gel and purified by flash chromatography using 98/2 CH₂Cl₂/ACN (to elute the unreacted p-toluenesulfonyl chloride) and then 2/1 hexanes/acetone to afford 2.70 g (24%) of the title compound. R_{f}=0.10 (98/2 CH₂Cl₂/ACN). ¹H NMR (400 MHz, CDCl₃); MS (ES⁺) m/z mass calcd for C₁₂H₁₇O₄S 258, found 259 (M+1, 100%).

### Step B

### Acetic acid 1-methyl-4-(toluene-4-sulfonyloxy)-butyl ester

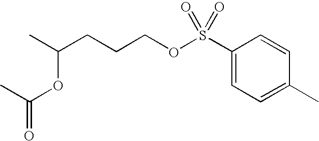

A solution of toluene-4-sulfonic acid 4-hydroxy-pentyl ester (1.21 g, 4.68 mmol), Et₃N (0.947 g, 9.36 mmol) and N,N-dimethylaminopyridine (0.114 g, 0.933 mmol) in CH₂Cl₂ (20 mL) is treated dropwise with acetic anhydride (0.572 g, 5.61 mmol), and the resultant mixture is stirred at rt for 2 hours under N₂. The reaction is quenched with 1 N HCl (15 mL), diluted with water and extracted with CH₂Cl₂. The organic layer is dried (Na₂SO₄) and the solvent is removed in vacuo to afford 0.803 g (57%) of the title compound that is utilized without purification. R_{f}=0.43 (1/1 hexanes/EtOAc). ¹H NMR (400 MHz, CDCl₃); MS (ES⁺) m/z mass calcd for C₁₄H₂₀O₅S 300, found 318 (M+NH₄, 100%).

### Step C

### [5-Ethyl-2-(4-hydroxy-pentyloxy)-phenyl]-phenyl-methanone

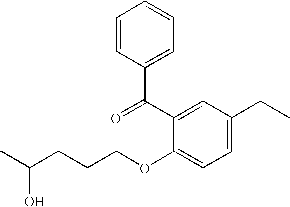

A mixture of (5-ethyl-2-methoxy-phenyl)-phenyl-methanone (0.248 g, 1.09 mmol), acetic acid 1-methyl-4-(toluene-4-sulfonyloxy)-butyl ester (0.362 g, 1.21 mmol) and Cs₂CO₃ (0.535 g, 1.64 mmol) in dry DMF (15 mL) is heated to 50° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford 0.750 g of crude product that is dissolved in methanol (10 mL) and treated with 325 mesh K₂CO₃ (0.302 g, 2.19 mmol). The mixture is stirred at rt until O-acyl protected intermediate R_{f}=0.23 (4/1 hexanes/EtOAc) is converted to product. The reaction is acidified with 1N HCl, diluted with water and extracted with EtOAc. The organic layer is dried (Na₂SO₄) and the solvent is removed in vacuo to afford crude product that is absorbed on silica gel and purified by column chromatography using a gradient of 3/1 then 1/1 hexanes/EtOAc to afford 0.233 g (68%) of the title compound. ¹H NMR (400 MHz, CDCl₃); MS (ES⁺) m/z mass calcd for C₂₀H₂₄O₃ 312, found 313 (M+1, 100%).

### Step D

### Methanesulfonic acid 4-(2-benzoyl-4-ethyl-phenoxy)-1-methyl-butyl ester

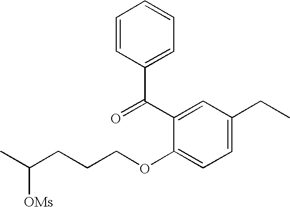

A 0° C. solution of [5-ethyl-2-(4-hydroxy-pentyloxy)-phenyl]-phenyl-methanone (0.233 g, 0.746 mmol) and TEA (0.189 g, 1.87 mmol) in CH₂Cl₂ (10 mL) is treated with MsCl (0.127 g, 1.11 mmol), and the reaction stirred for 2 hours at 0° C. under N₂. The reaction is quenched with 1 N HCl (4 mL) and diluted with additional CH₂Cl₂ and extracted with water. The organic layer is dried (MgSO₄), and the solvent is removed in vacuo to afford 0.310 g (100%) of the title compound that is utilized without purification. R_{f}=0.35 (1/1 hexanes/EtOAc). ¹H NMR (400 MHz, CDCl₃); MS (ES⁺) m/z mass calcd for C₂₁H₂₆O₅S 390, found 391 (M+1, 100%).

### Step E

### 3-{4-[4-(2-Benzoyl-4-ethyl-phenoxy)-1-methyl-butoxy]-2-methyl-phenyl}-propionic acid methyl ester

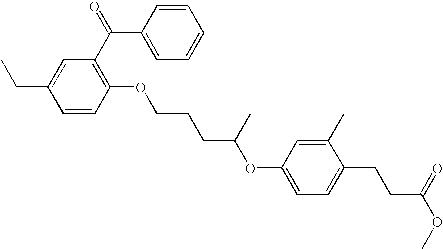

A mixture of 3-(4-hydroxy-2-methyl-phenyl)-propionic acid methyl ester (0.135 g, 0.695 mmol), methanesulfonic acid 4-(2-benzoyl-4-ethyl-phenoxy)-1-methyl-butyl ester (0.30 g, 0.768 mmol) and cesium carbonate (0.341 g, 1.05 mol) in dry DMF (10 mL) is heated to 60° C. for 17 hours under N₂. The reaction is cooled and quenched with 1 N HCl (20 mL). The mixture is diluted with Et₂O and extracted with water. The organic layer is dried (Na₂SO₄), and the solvent is removed in vacuo to afford crude product that is absorbed on silica gel and purified by flash chromatography using 98/2 CH₂Cl₂/ACN to afford 0.208 g (61%) of the title compound. R_{f}=0.52 (98/2 CH₂Cl₂/ACN). ¹H NMR (400 MHz, CDCl₃); MS (ES⁺) m/z mass calcd for C₃₁H₃₆O₅ 488, found 489 (M+1, 100%).

### Step F

### 3-{4-[4-(2-Benzoyl-4-ethyl-phenoxy)-1-methyl-butoxy]-2-methyl-phenyl}-propionic acid

A solution of 3-{4-[4-(2-benzoyl-4-ethyl-phenoxy)-1-methyl-butoxy]-2-methyl-phenyl}-propionic acid methyl ester (0.208, 0.426 mmol) in methanol (8 mL) is treated with 5 N NaOH (2 mL) and stirred at rt until saponification complete. The solvent is removed in vacuo to afford a residue that is acidified with 1 N HCl. The mixture is diluted with water and extracted with EtOAc. The organic layer is dried (Na₂SO₄), and the solvent is removed in vacuo to afford 0.168 g of acid that is purified by preparative HPLC to give 0.099 g (49%) of the title compound. ¹H NMR (400 MHz, CDCl₃); MS (ES⁺) m/z mass calcd for C₃₀H₃₄O₅ 474, found 475 (M+1, 100%).

### EXAMPLE 130

### 3-{4-[4-(2-Benzoyl-4-ethyl-phenoxy)-pentyloxy]-2-methyl-phenyl}-propionic acid

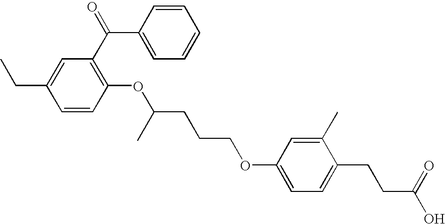

### Step A

### 3-[4-(4-Hydroxy-pentyloxy)-2-methyl-phenyl]-propionic acid methyl ester

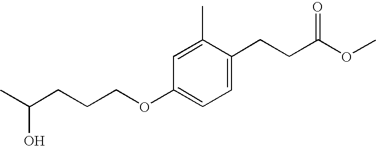

A mixture of 3-(4-hydroxy-2-methyl-phenyl)-propionic acid methyl ester (0.247 g, 1.27 mmol), acetic acid 1-methyl-4-(toluene-4-sulfonyloxy)-butyl ester (0.421 g, 1.40 mmol) and Cs₂CO₃ (0.622 g, 1.91 mmol) in dry DMF (15 mL) is heated to 50° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford 0.735 g of crude product that is dissolved in methanol (10 mL) and treated with 325 mesh K₂CO₃ (0.351 g, 2.549 mmol). The mixture is stirred at rt until O-acyl protected intermediate R_{f}=0.53 (1/1 hexanes/EtOAc) is converted to product. The reaction is acidified with 1 N HCl, diluted with water and extracted with EtOAc. The organic layer is dried (Na₂SO₄) and the solvent is removed in vacuo to afford crude product that is absorbed on silica gel and purified by column chromatography using a gradient of 3/1 then 1/1 hexanes/EtOAc to afford 0.150 g (42%) of the title compound. R_{f}=0.27 (1/1 hexanes/EtOAc). ¹H NMR (400 MHz, CDCl₃); MS (ES⁺) m/z mass calcd for C₁₆H₂₄O₄ 280, found 303 (M+Na, 100%).

### Step B

### 3-[4-(4-Methanesulfonyloxy-pentyloxy)-2-methyl-phenyl]-propionic acid methyl ester

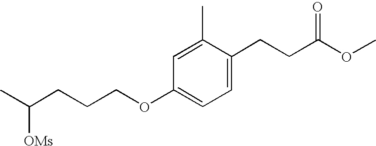

A 0° C. solution of 3-[4-(4-hydroxy-pentyloxy)-2-methyl-phenyl]-propionic acid methyl ester (0.150 g, 0.535 mmol) and TEA (0.135 g, 1.33 mmol) in CH₂Cl₂ (8 mL) is treated with MsCl (0.092 g, 0.801 mmol), and the reaction stirred for 2 hours at 0° C. under N₂. The reaction is quenched with 1 N HCl (4 mL) and diluted with more CH₂Cl₂ and extracted with water. The organic layer is dried (Na₂SO₄), and the solvent is removed in vacuo to afford 0.185 g (96%) of the title compound that is utilized without purification. R_{f}=0.38 (1/1 hexanes/EtOAc). ¹H NMR (400 MHz, CDCl₃); MS (ES⁺) m/z mass calcd for C₁₇H₂₆O₆S 358, found 376 (M+NH₄, 100%).

### Step C

### 3-{4-[4-(2-Benzoyl-4-ethyl-phenoxy)-pentyloxy]-2-methyl-phenyl}-propionic acid methyl ester

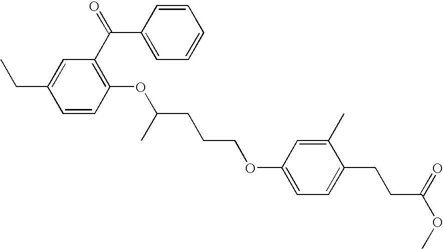

A mixture of (5-ethyl-2-methoxy-phenyl)-phenyl-methanone (0.106 g, 0.469 mmol), 3-[4-(4-methanesulfonyloxy-pentyloxy)-2-methyl-phenyl]-propionic acid methyl ester (0.185 g, 0.516 mmol) and cesium carbonate (0.229 g, 0.703 mol) in dry DMF (10 mL) is heated to 60° C. for 17 hours under N₂. The reaction is cooled and quenched with 1 N HCl (20 mL). The mixture is diluted with Et₂O and extracted with water. The organic layer is dried (Na₂SO₄) and the solvent is removed in vacuo to afford crude product that is absorbed on silica gel and purified by flash chromatography using 9/1 hexanes/EtOAc to afford 0.114 g of the title compound. R_{f}=0.50 (98/2 CH₂Cl₂/ACN). ¹H NMR (400 MHz, CDCl₃); MS (ES⁺) m/z mass calcd for C₃₁H₃₆O₅ 488, found 489 (M+1, 100%).

### Step D

### 3-{4-[4-(2-Benzoyl-4-ethyl-phenoxy)-pentyloxy]-2-methyl-phenyl}-propionic acid

A solution of 3-{4-[4-(2-benzoyl-4-ethyl-phenoxy)-pentyloxy]-2-methyl-phenyl}-propionic acid methyl ester (0.114, 0.233 mmol) in methanol (8 mL) is treated with 5 N NaOH (2 mL) and stirred at rt until saponification is completed. The solvent is removed in vacuo to afford a residue that is acidified with 1 N HCl. The mixture is diluted with water and extracted with EtOAc. The organic layer is dried (Na₂SO₄), and the solvent is removed in vacuo to afford 0.111 g (100%) of the title compound. ¹H NMR (400 MHz, CDCl₃); MS (ES⁺) m/z mass calcd for C₃₀H₃₄O₅ 474, found 475 (M+1, 100%).

### EXAMPLE 131

### 3-{4-[3-(2-Benzoyl-4-ethyl-phenoxy)-2-methyl-propoxy]-2-methyl-phenyl}-propionic acid

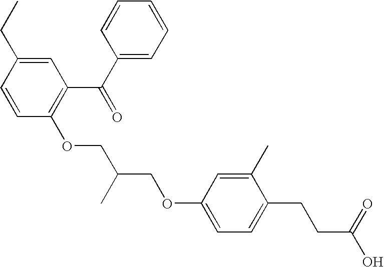

### Step A

Methanesulfonic acid 3-methanesulfonyloxy-2-methyl-propyl ester

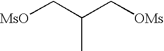

A 0° C. solution of 2-methyl-propane-1,3-diol (10.0 g, 0.111 mol) and Et₃N (39.3 g, 0.388 mol) in CH₂Cl₂ (200 mL) is treated dropwise with MsCl (33.0 g, 0.228 mol) and stirred at 0° C. for 3 hours under N₂. The reaction is quenched with 1 N HCl (300 mL), diluted with CH₂Cl₂ and then extracted with water. The organic layer is dried (Na₂SO₄), and the solvent is removed in vacuo to afford 26.74 g (98%) of the title compound. ¹H NMR (400 MHz, CDCl₃); MS (ES⁺) m/z mass calcd for C₆H₁₄O₆S₂ 246, found 264 (M+NH₄, 100%).

### Step B

### Methanesulfonic acid 3-(2-benzoyl-4-ethyl-phenoxy)-2-methyl-propyl ester

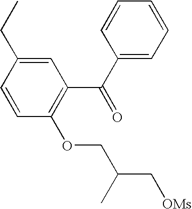

A mixture of (5-ethyl-2-methoxy-phenyl)-phenyl-methanone (1.00 g, 4.42 mmol) methanesulfonic acid 3-methanesulfonyloxy-2-methyl-propyl ester (8.71 g, 35.4 mmol) and cesium carbonate (2.16 g, 6.63 mol) in dry DMF (30 mL) is heated to 50° C. for 17 hours under N₂. The reaction is cooled and acidified with 1 N HCl. The mixture is diluted with Et₂O and extracted with water. The organic layer is dried (Na₂SO₄), and the solvent is removed in vacuo to afford crude product that is absorbed on silica gel and purified by flash chromatography using 6/1 hexanes/acetone to afford 1.76 g (100%) of the title compound. R_{f}=0.10 (2/1 hexanes/acetone). ¹H NMR (400 MHz, CDCl₃); MS (ES⁺) m/z mass calcd for C₂₀H₂₄O₅S 376, found 377 (M+1, 100%).

### Step C

### 3-{4-[3-(2-Benzoyl-4-ethyl-phenoxy)-2-methyl-propoxy]-2-methyl-phenyl}-propionic acid methyl ester

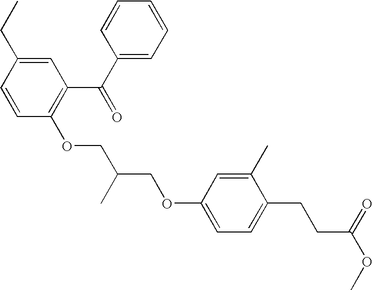

A mixture of 3-(4-hydroxy-2-methyl-phenyl)-propionic acid methyl ester (0.102 g, 0.525 mmol, methanesulfonic acid 3-(2-benzoyl-4-ethyl-phenoxy)-2-methyl-propyl ester (0.197 g, 0.523 mmol) and cesium carbonate (0.205 g, 0.629 mmol) in dry DMF (10 mL) is heated to 50° C. for 17 hours under N₂. The reaction is cooled and acidified with 1 N HCl (25 mL). The mixture is diluted with Et₂O and extracted with water. The organic layer is dried (Na₂SO₄), and the solvent is removed in vacuo to afford crude product that is absorbed on silica gel and purified by flash chromatography using 10/1 hexanes/acetone to afford 0.105 g (42%) of the title compound. R_{f}=0.23 (2/1 hexanes/acetone). ¹H NMR (400 MHz, CDCl₃); MS (ES⁺) m/z mass calcd for C₃₀H₃₄O₅ 474, found 475 (M+1, 100%).

### Step D

### 3-{4-[3-(2-Benzoyl-4-ethyl-phenoxy)-2-methyl-propoxy]-2-methyl-phenyl}-propionic acid

A solution of 3-{4-[3-(2-benzoyl-4-ethyl-phenoxy)-2-methyl-propoxy]-2-methyl-phenyl}-propionic acid methyl ester (0.105 g, 0.221 mmol) in methanol (6 mL) is treated with 5 N NaOH (1 mL) and stirred at rt until saponification is completed. The mixture is acidified with 1 N HCl, diluted with water, and extracted with Et₂O. The organic layer is dried (Na₂SO₄), and the solvent is removed in vacuo to afford 0.116 g (100%) of the title compound. ¹H NMR (400 MHz, CDCl₃); HRMS (ES⁺) m/z exact mass calcd for C₂₉H₃₃O₅ 461.2328, found 461.2328.

### EXAMPLE 132

### 3-{4-[3-(2-Benzoyl-4-ethyl-phenoxy)-propoxy]-2-methyl-phenyl}-propionic acid

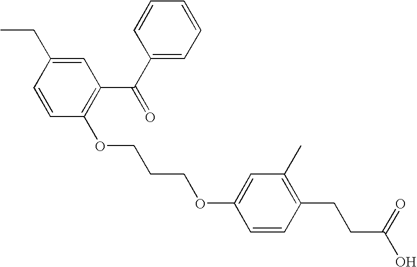

### Step A

### 3-[4-(3-Hydroxy-propoxy)-2-methyl-phenyl]-propionic acid methyl ester

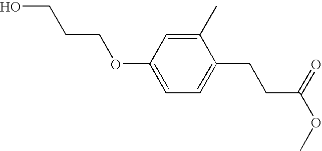

A mixture of 3-(4-hydroxy-2-methyl-phenyl)-propionic acid methyl ester (5.00 g, 25.7 mmol) 3-bromo-propan-1-ol (5.37 g, 38.6 mmol) and cesium carbonate (12.6 g, 38.7 mol) in dry DMF (50 mL) is heated to 50° C. for 17 hours under N₂. The reaction is cooled and filtered, and the filtrate is quenched with 1 N HCl (50 mL). The filtrate is then diluted with Et₂O and extracted with water. The organic layer is dried (Na₂SO₄), and the solvent is removed in vacuo to afford crude product that is absorbed on silica gel and purified by flash chromatography using 6/1 hexanes/EtOAc to afford 2.08 g (32%) of the title compound. R_{f}=0.30 (1/1 hexanes/acetone). ¹H NMR (400 MHz, CDCl₃); MS (ES⁺) m/z mass calcd for C₁₄H₂₀O₄ 252, found 253 (M+1, 100%).

### Step B

### 3-[4-(3-Methanesulfonyloxy-propoxy)-2-methyl-phenyl]-propionic acid methyl ester

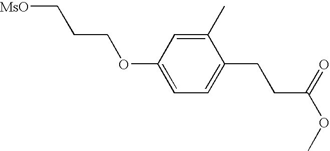

A 0° C. solution of 3-[4-(3-hydroxy-propoxy)-2-methyl-phenyl]-propionic acid methyl ester (2.05 g, 8.12 mmol) and Et₃N (1.23 g, 12.2 mmol) in CH₂Cl₂ (30 mL) is treated dropwise with MsCl (1.11 g, 9.69 mmol) and stirred at 0° C. for 1 hour under N₂. The reaction is quenched with 1 N HCl (15 mL), diluted with CH₂Cl₂ and then extracted with water. The organic layer is dried (Na₂SO₄), and the solvent is removed in vacuo to afford 2.73 g (100%) of the title compound. ¹H NMR (400 MHz, CDCl₃); MS (ES⁺) m/z mass calcd for C₁₅H₂₂O₆S 330, found 348 (M+NH₄, 100%).

### Step C

### 3-{4-[3-(2-Benzoyl-4-ethyl-phenoxy)-propoxy]-2-methyl-phenyl}-propionic acid

A mixture of (5-ethyl-2-methoxy-phenyl)-phenyl-methanone (0.068 g, 0.301 mmol), 3-[4-(3-methanesulfonyloxy-propoxy)-2-methyl-phenyl]-propionic acid methyl ester (0.100 g, 0.303 mmol) and Cs₂CO₃ (0.118 g, 0.362 mmol) in dry DMF (7 mL) is heated to 50° C. and stirred for 17 hours under N₂. The reaction is treated with 5 N NaOH (2 mL), cooled and stirred at rt until saponification is completed. The mixture is acidified with 1 N HCl, diluted with water, and extracted with Et₂O. The organic layer is dried (Na₂SO₄) and the solvent is removed in vacuo to afford 0.479 g of crude acid that is purified by preparative HPLC to give 0.063 g (47%) of the title compound. ¹H NMR (400 MHz, CDCl₃); MS (ES⁺) m/z mass calcd for C₂₈H₃₀O₅ 446, found 447 (M+1, 100%).

### EXAMPLE 133

### 3-(4-{3-[4-Ethyl-2-(4-fluoro-benzoyl)-phenoxy]-propoxy}-2-methyl-phenyl)-propionic acid

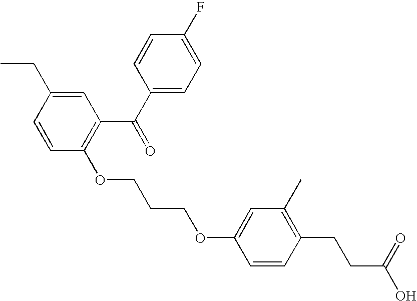

### Step A

### (5-Ethyl-2-methoxy-phenyl)-(4-fluoro-phenyl)-methanone

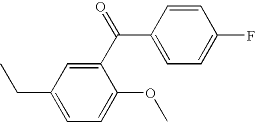

The procedure from Example 101, Step A is utilized with 4-fluoromethyl-benzoyl chloride to afford 10.2 g (100%) of the title compound. ¹H NMR (400 MHz, CDCl₃); MS (ES⁺) m/z mass calcd for C₁₆H₁₅O₂F 258, found 259 (M+1, 100%).

### Step B

### (5-Ethyl-2-hydroxy-phenyl)-(4-fluoro-phenyl)-methanone

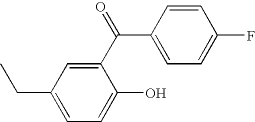

The procedure from Example 101, Step B is utilized with (5-ethyl-2-methoxy-phenyl)-(4-fluoro-phenyl)-methanone to afford 4.15 g (88%) of the title compound. ¹H NMR (400 MHz, CDCl₃); MS (ES⁻) m/z mass calcd for C₁₅H₁₃O₂F 244, found 243 (M−1, 100%).

### Step C

### 3-(4-{3-[4-Ethyl-2-(4-fluoro-benzoyl)-phenoxy]-propoxy}-2-methyl-phenyl)-propionic acid

The procedure from Example 132, Step C is utilized with (5-ethyl-2-hydroxy-phenyl)-(4-fluoro-phenyl)-methanone to afford 0.081 g (48%) of the title compound. ¹H NMR (400 MHz, CDCl₃); MS (ES⁺) m/z mass calcd for C₂₈H₂₉O₅F 464, found 465 (M+1,100%).

### EXAMPLE 134

### 3-(4-{3-[4-Ethyl-2-(4-trifluoromethyl-benzoyl)-phenoxy]-propoxy}-2-methyl-phenyl)-propionic acid

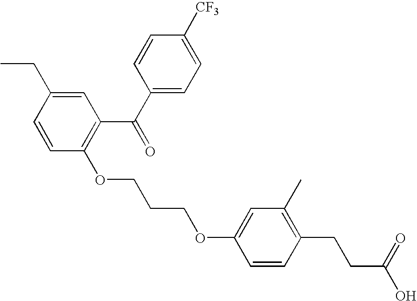

### Step A

### (5-Ethyl-2-methoxy-phenyl)-(4-trifluoromethyl-phenyl)-methanone

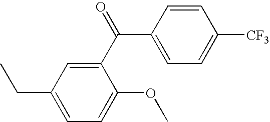

The procedure from Example 101, Step A is utilized with 4-trifluoromethyl-benzoyl chloride to afford 3.39 g (75%) of the title compound. ¹H NMR (400 MHz, CDCl₃); MS (ES⁺) m/z mass calcd for C₁₇H₁₅O₂F₃ 308, found 309 (M+1, 100%).

### Step B

### (5-Ethyl-2-hydroxy-phenyl)-(4-trifluoromethyl-phenyl)-methanone

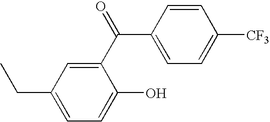

The procedure from Example 101, Step B is utilized with (5-ethyl-2-methoxy-phenyl)-(4-trifluoromethyl-phenyl)-methanone to afford 3.2 g (100%) of the title compound. ¹H NMR (400 MHz, CDCl₃); MS (ES⁻) m/z mass calcd for C₁₆H₁₃O₂F₃ 294, found 293 (M−1, 100%).

### Step C

### 3-(4-{3-[4-Ethyl-2-(4-trifluoromethyl-benzoyl)-phenoxy]-propoxy}-2-methyl-phenyl)-propionic acid

The procedure from Example 132, Step C is utilized with (5-ethyl-2-hydroxy-phenyl)-(4-trifluoromethyl-phenyl)-methanone to afford 0.079 g (51%) of the title compound. ¹H NMR (400 MHz, CDCl₃); MS (ES⁺) m/z mass calcd for C₂₉H₂₉O₅F₃ 514, found 515 (M+1, 100%).

### EXAMPLE 135

### 3-(4-{3-[4-Ethyl-2-(3-trifluoromethyl-benzoyl)-phenoxy]-propoxy}-2-methyl-phenyl)-propionic acid

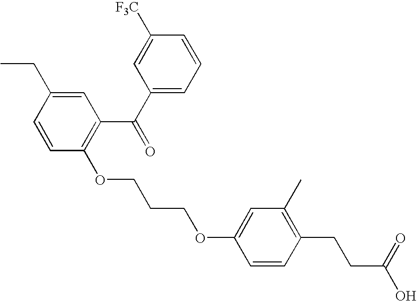

### Step A

### (5-Ethyl-2-methoxy-phenyl)-(3-trifluoromethyl-phenyl)-methanone

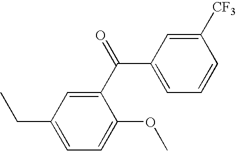

The procedure from Example 101, Step A is utilized with 3-trifluoromethyl-benzoyl chloride to afford 3.90 g (45%) of the title compound. ¹H NMR (400 MHz, CDCl₃); MS (ES⁺) m/z mass calcd for C₁₇H₁₅O₂F₃ 308, found 309 (M+1, 100%).

### Step B

### (5-Ethyl-2-hydroxy-phenyl)-(3-trifluoromethyl-phenyl)-methanone

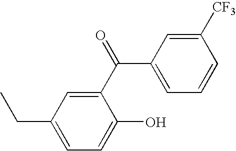

The procedure from Example 101, Step B is utilized with (5-ethyl-2-methoxy-phenyl)-(3-trifluoromethyl-phenyl)-methanone to prepare 3.46 g (93%) of the title compound. ¹H NMR (400 MHz, CDCl₃); MS (ES⁻) m/z mass calcd for C₁₆H₁₃O₂F₃ 294, found 293 (M−1, 100%).

### Step C

### 3-(4-{3-[4-Ethyl-2-(3-trifluoromethyl-benzoyl)-phenoxy]-propoxy}-2-methyl-phenyl)-propionic acid

The procedure from Example 132, Step C is utilized with (5-ethyl-2-hydroxy-phenyl)-(3-trifluoromethyl-phenyl)-methanone to afford 0.069 g (30%) of the title compound. ¹H NMR (400 MHz, CDCl₃); MS (ES⁺) m/z mass calcd for C₂₉H₂₉O₅F₃ 514, found 515 (M+1, 100%).

### EXAMPLE 136

### 3-(4-{3-[4-Ethyl-2-(2-trifluoromethyl-benzoyl)-phenoxy]-propoxy}-2-methyl-phenyl)-propionic acid

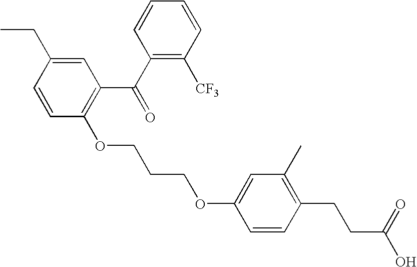

### Step A

### (5-Ethyl-2-methoxy-phenyl)-(2-trifluoromethyl-phenyl)-methanone

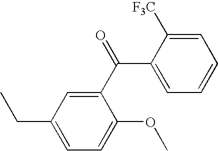

The procedure from Example 101, Step A is utilized with 2-trifluoromethyl-benzoyl chloride to prepare 5.18 g (100%) of the title compound. ¹H NMR (400 MHz, CDCl₃); MS (ES⁺) m/z mass calcd for C₁₇H₁₅O₂F₃ 308, found 309 (M+1, 100%).

### Step B

### (5-Ethyl-2-hydroxy-phenyl)-(2-trifluoromethyl-phenyl)-methanone

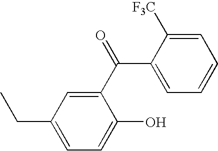

The procedure from Example 101, Step B is utilized with (5-ethyl-2-methoxy-phenyl)-(2-trifluoromethyl-phenyl)-methanone to afford 4.17 g (93%) of the title compound. ¹H NMR (400 MHz, CDCl₃); MS (ES⁻) m/z mass calcd for C₁₆H₁₃O₂F₃ 294, found 293 (M−1, 100%).

### Step C

### 3-(4-{3-[4-Ethyl-2-(2-trifluoromethyl-benzoyl)-phenoxy]-propoxy}-2-methyl-phenyl)-propionic acid

The procedure from Example 132, Step C is utilized with (5-ethyl-2-hydroxy-phenyl)-(2-trifluoromethyl-phenyl)-methanone to afford 0.025 g (16%) of the title compound. ¹H NMR (400 MHz, CDCl₃); MS (ES⁺) m/z mass calcd for C₂₉H₂₉O₅F₃ 514, found 515 (M+1, 100%).

### EXAMPLE 137

### 3-(4-{3-[4-Ethyl-2-(thiophene-2-carbonyl)-phenoxy]-propoxy}-2-methyl-phenyl)-propionic acid

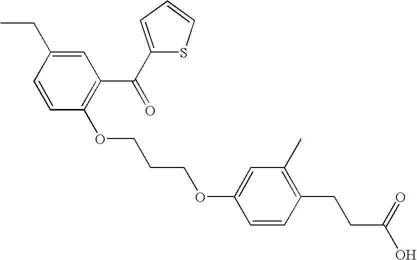

The title compound is prepared according to the procedure described in Example 132, Step C by utilizing (5-ethyl-2-hydroxy-phenyl)-thiophen-2-yl-methanone to afford 0.101 g (66%). ¹H NMR (400 MHz, CDCl₃); MS (ES⁺) m/z mass calcd for C₂₆H₂₈O₅S 452, found 453 (M+1, 100%).

### EXAMPLE 138

### 3-{4-[3-(2-Benzyl-4-ethyl-phenoxy)-propoxy]-2-methyl-phenyl}-propionic acid

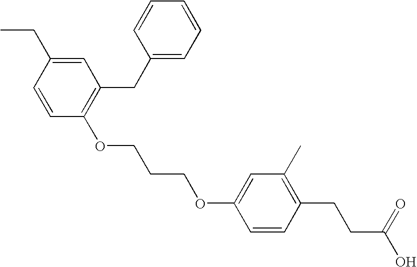

The title compound is prepared according to the procedure described in Example 132, Step C by utilizing 2-benzyl-4-ethyl-phenol to afford 0.063 g (49%). ¹H NMR (400 MHz, CDCl₃); MS (ES⁺) m/z mass calcd for C₂₈H₃₂O₄ 432, found 433 (M+1, 100%).

### EXAMPLE 139

### 3-(4-{3-[4-Ethyl-2-(naphthalene-1-carbonyl)-phenoxy]-propoxy}-2-methyl-phenyl)-propionic acid

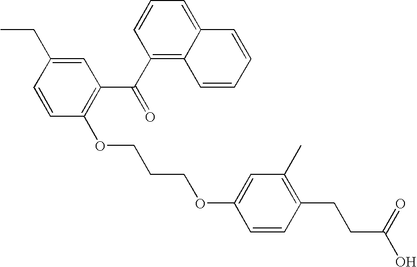

The title compound is prepared according to the procedure described in Example 132, Step C by utilizing (5-ethyl-2-hydroxy-phenyl)-naphthalen-1-yl-methanone to afford 0.067 g (48%). ¹H NMR (400 MHz, CDCl₃); MS (ES⁺) m/z mass calcd for C₃₂H₃₂O₅ 496, found 497 (M+1, 100%).

### EXAMPLE 140

### 3-(4-{3-[4-Ethyl-2-(1-phenyl-vinyl)-phenoxy]-propoxy}-2-methyl-phenyl)-propionic acid

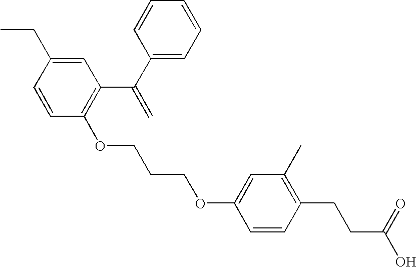

The title compound is prepared according to the procedure described in Example 132, Step C by utilizing 4-ethyl-2-(1-phenyl-vinyl)-phenol to afford 0.030 g (21%). ¹H NMR (400 MHz, CDCl₃); MS (ES⁻) m/z mass calcd for C₂₉H₃₂O₄ 444, found 443 (M−1, 100%).

### EXAMPLE 141

### 3-(4-{3-[2-(Benzo[b]thiophene-2-carbonyl)-4-ethyl-phenoxy]-propoxy}-2-methyl-phenyl)-propionic acid

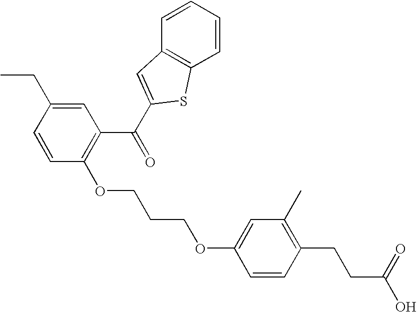

The title compound is prepared according to the procedure described in Example 132, Step C by utilizing benzo[b]thiophen-2-yl-(5-ethyl-2-hydroxy-phenyl)-methanone to afford 0.119 g (88%). ¹H NMR (400 MHz, CDCl₃); HRMS (ES⁺) m/z exact mass calcd for C₃₀H₃₁O₅S 503.1892, found 503.1890.

### EXAMPLE 142

### 2-{4-[3-(2-Benzoyl-4-ethyl-phenoxy)-butoxy]-phenoxy}-2-methyl-propionic acid

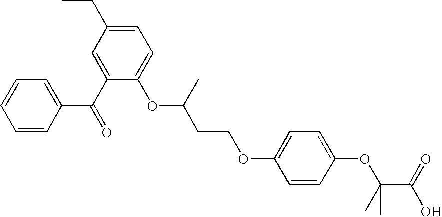

A mixture of (5-ethyl-2-methoxy-phenyl)-phenyl-methanone (0.070 g, 0.309 mmol), 2-[4-(3-methanesulfonyloxy-butoxy)-phenoxy]-2-methyl-propionic acid ethyl ester (0.115 g, 0.307 mmol) and Cs₂CO₃ (0.121 g, 0.371 mmol) in dry DMF (7 mL) is heated to 50° C. and stirred for 17 hours under N₂. The reaction is cooled to rt and acidified with 1 N HCl. The mixture is diluted with Et₂O and extracted with water. The organic layer is dried (Na₂SO₄), and the solvent is removed in vacuo to afford crude ester that is dissolved in ethanol (6 mL) and treated with 5 N NaOH (0.50 mL). The mixture is stirred at rt until saponification is completed. The mixture is acidified with 1 N HCl, diluted with water, and the mixture extracted with EtOAc. The organic layer is dried (Na₂SO₄), and the solvent is removed in vacuo to afford crude acid that is purified by preparative HPLC to give 0.024 g (16%) of the title compound. ¹H NMR (400 MHz, CDCl₃); HRMS (ES⁺) m/z exact mass calcd for C₂₉H₃₃O₆ 477.2277, found 477.2264.

### EXAMPLE 143

### 2-{4-[3-(2-Benzoyl-4-ethyl-phenoxy)-2-methyl-propoxy]-phenoxy}-2-methyl-propionic acid

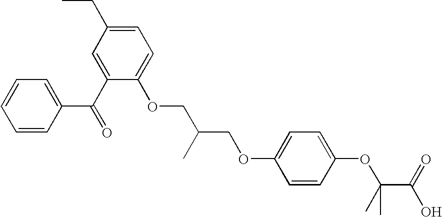

The title compound is prepared according to the procedure described in Example 142 by utilizing 2-[4-(3-methanesulfonyloxy-2-methyl-propoxy)-phenoxy]-2-methyl-propionic acid ethyl ester to afford 0.059 g (41%). ¹H NMR (400 MHz, CDCl₃); HRMS (ES⁺) m/z exact mass calcd for C₂₉H₃₃O₆ 477.2277, found 477.2258.

### EXAMPLE 144

### 2-{4-[3-(2-Benzyl-4-ethyl-phenoxy)-butoxy]-phenoxy}-2-methyl-propionic acid

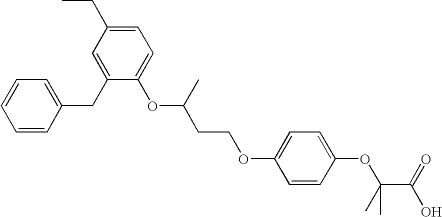

The title compound is prepared according to the procedure described in Example 142 by utilizing 2-[4-(3-methanesulfonyloxy-butoxy)-phenoxy]-2-methyl-propionic acid ethyl ester and 2-benzyl-4-ethyl-phenol to afford 0.048 g (20%). ¹H NMR (400 MHz, CDCl₃); HRMS (ES⁺) m/z exact mass calcd for C₂₉H₃₄O₅Na 485.2304, found 485.2299.

### EXAMPLE 145

### 2-{4-[3-(2-Benzoyl-4-bromo-phenoxy)-butoxy]-phenoxy}-2-methyl-propionic acid

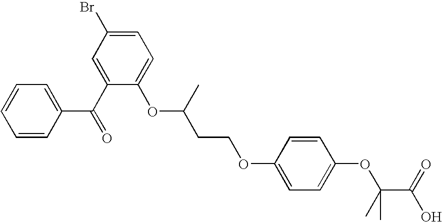

### Step A

### 2-{4-[3-(2-Benzoyl-4-bromo-phenoxy)-butoxy]-phenoxy}-2-methyl-propionic acid ethyl ester

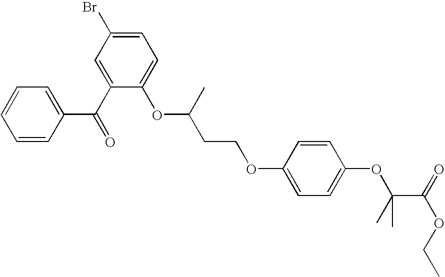

A mixture of 2-[4-(3-methanesulfonyloxy-butoxy)-phenoxy]-2-methyl-propionic acid ethyl ester (0.405 g, 1.08 mmol), (5-bromo-2-hydroxy-phenyl)-phenyl-methanone (0.250 g, 0.902 mmol) and cesium carbonate (0.382 g, 1.17 mmol) in dry DMF (25 mL) is heated to 50° C. for 6 hours under N₂. The reaction is cooled and acidified with 1 N HCl (30 mL). The mixture is diluted with Et₂O and extracted with water. The organic layer is dried (Na₂SO₄) and the solvent is removed in vacuo to afford crude product that is absorbed on silica gel and purified by flash chromatography using 8/1 hexanes/acetone to afford 0.184 g (30%) of the title compound. R_{f}=0.35 (2/1 hexanes/acetone). ¹H NMR (400 MHz, CDCl₃).

### Step B

### 2-{4-[3-(2-Benzoyl-4-bromo-phenoxy)-butoxy]-phenoxy}-2-methyl-propionic acid

A solution of 2-{4-[3-(2-benzoyl-4-bromo-phenoxy)-butoxy]-phenoxy}-2-methyl-propionic acid ethyl ester (0.059 g, 0.106 mmol) in ethanol (6 mL) is treated with 5 N NaOH (0.5 mL) and stirred at rt until saponification is completed. The solvent is removed in vacuo to afford a residue that is acidified with 1 N HCl, diluted with water, and extracted with EtOAc. The organic layer is dried (Na₂SO₄) and the solvent is removed in vacuo to afford 0.049 g of acid that is purified by preparative HPLC to give 0.044 g (79%) of the title compound. ¹H NMR (400 MHz, CDCl₃); MS (ES⁺) m/z mass calcd for C₂₇H₂₇O₆Br 526, found 527 and 529 (M+1 and M+3, 100%).

### EXAMPLE 146

### 2-{4-[3-(2-Benzoyl-4-butyl-phenoxy)-butoxy]-phenoxy}-2-methyl-propionic acid

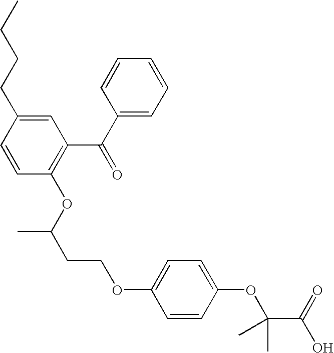

### Step A

### 2-{4-[3-(2-Benzoyl-4-butyl-phenoxy)-butoxy]-phenoxy}-2-methyl-propionic acid ethyl ester

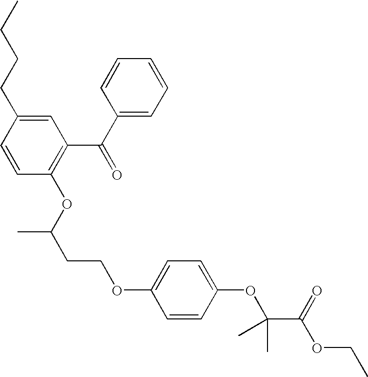

The compounds of 2-{4-[3-(2-benzoyl-4-bromo-phenoxy)-butoxy]-phenoxy}-2-methyl-propionic acid ethyl ester (Example 145, Step A) (0.118 g, 0.212 mmol), n-butylboronic acid (0.065 g, 0.638 mmol) and cesium fluoride (0.113 g, 0.744 mmol) are combined in 1,4-dioxane (6 mL) and purged with N₂. The reaction is treated with 1,1′-bis(diphenylphosphino)ferrocene palladium (II)chloride, CH₂Cl₂ complex (0.023 g, 0.031 mmol) and heated in an oil bath at 80° C. for 10 hours under N₂. The reaction is cooled and the solvent is removed in vacuo to afford crude product that is absorbed on silica gel and column purified using 8/1 hexanes/acetone to afford 0.078 g (69%) of the title compound. R_{f}=0.28 (2/1 hexanes/acetone). ¹H NMR (400 MHz, CDCl₃); MS (ES⁺) m/z mass calcd for C₃₃H₄₀O₆ 532, found 533 (M+1, 100%).

### Step B

### 2-{4-[3-(2-Benzoyl-4-butyl-phenoxy)-butoxy]-phenoxy}-2-methyl-propionic acid

A solution of 2-{4-[3-(2-benzoyl-4-butyl-phenoxy)-butoxy]-phenoxy}-2-methyl-propionic acid ethyl ester (0.078, 0.146 mmol) in ethanol (6 mL) is treated with 5 N NaOH (0.5 mL) and stirred at rt until saponification complete. The solvent is removed in vacuo to afford a residue that is acidified with 1 N HCl. The mixture is diluted with water and extracted with EtOAc. The organic layer is dried (Na₂SO₄) and the solvent is removed in vacuo to afford 0.084 g (100%) of the title compound. ¹H NMR (400 MHz, CDCl₃); HRMS (ES⁺) m/z exact mass calcd for C₃₁H₃₇O₆ 505.2590, found 505.2617.

### EXAMPLE 147

### 2-Methyl-2-{4-[3-(3-phenyl-benzofuran-6-yloxy)-hexyloxy]-phenoxy}-propionic acid

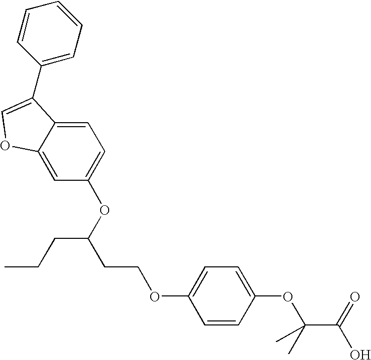

### Step A

### 3-(3-Phenyl-benzofuran-6-yloxy)-hexan-1-ol

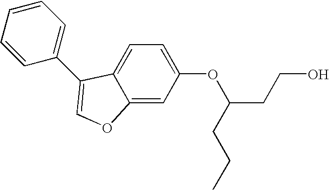

A mixture of (3-phenyl-benzofuran-6-ol (0.36 g, 1.71 mmol), 3-bromo-hexan-1-ol (0.403 g, 2.23 mmol) (Example 101, Step C) and Cs₂CO₃ (0.837 g, 2.57 mmol) in dry DMF (15 mL) is heated to 50° C. and stirred for 17 hours under N₂. The reaction is cooled and acidified with 1 N HCl (12 mL). The mixture is diluted with water and extracted with Et₂O. The organic layer is dried (Na₂SO₄), and the solvent is removed in vacuo to afford crude product that is absorbed on silica gel and purified by column chromatography using 97/3 CH₂Cl₂/ACN to afford 0.109 g (20%) of the title compound. ¹H NMR (400 MHz, CDCl₃); MS (ES⁺) m/z mass calcd for C₂₀H₂₂O₃ 310, found 311 (M+1, 100%).

### Step B

### Methanesulfonic acid 3-(3-phenyl-benzofuran-6-yloxy)-hexyl ester

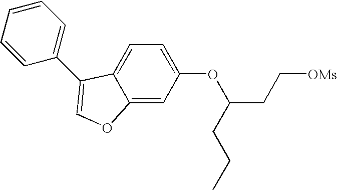

A 0° C. solution of 3-(3-phenyl-benzofuran-6-yloxy)-hexan-1-ol (0.109 g, 0.351 mmol) and TEA (0.053 g, 0.524 mmol) in CH₂Cl₂ (8 mL) is treated with MsCl (0.049 g, 0.426 mmol), and the reaction is stirred for 2 hours at 0° C. under N₂. The reaction is quenched with 1 N HCl (10 mL) and diluted with more CH₂Cl₂ and extracted with water. The organic layer is dried (MgSO₄), and the solvent is removed in vacuo to afford 0.146 g (100%) of the title compound that is utilized without purification. ¹H NMR (400 MHz, CDCl₃).

### Step C

### 2-Methyl-2-{4-[3-(3-phenyl-benzofuran-6-yloxy)-hexyloxy]-phenoxy}-propionic acid ethyl ester

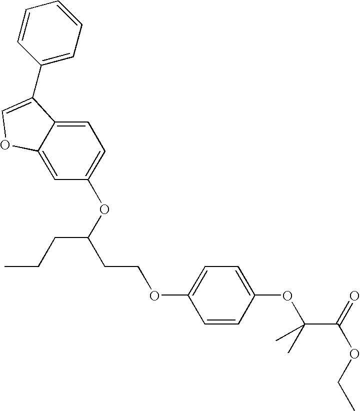

A mixture of (2-(4-hydroxy-phenoxy)-2-methyl-propionic acid ethyl ester (0.054 g, 0.241 mmol), methanesulfonic acid 3-(3-phenyl-benzofuran-6-yloxy)-hexyl ester (0.093 g, 0.239 mmol) and Cs₂CO₃ (0.117 g, 0.359 mmol) in dry DMF (7 mL) is heated to 50° C. and stirred for 17 hours under N₂. The reaction is cooled, quenched with 1 N HCl (12 mL), diluted with Et₂O and extracted with water. The organic layer is dried (Na₂SO₄) and the solvent is removed in vacuo to afford crude ester that is purified with column chromatography using 7/1 hexanes/acetone to afford 0.062 g (50%) of the title compound. R_{f}=0.38 (2/1 hexanes/acetone). ¹H NMR (400 MHz, CDCl₃); MS (ES⁺) m/z mass calcd for C₃₂H₃₆O₆ 516, found 517 (M+1, 100%).

### Step D

### 2-Methyl-2-{4-[3-(3-phenyl-benzofuran-6-yloxy)-hexyloxy]-phenoxy}-propionic acid

A solution of 2-methyl-2-{4-[3-(3-phenyl-benzofuran-6-yloxy)-hexyloxy]-phenoxy}-propionic acid ethyl ester (0.062 g, 0.120 mmol) in ethanol (6) is treated with 5 N NaOH (0.50 mL) and stirred at rt for 3 hours. The solvent is removed in vacuo to afford a residue that is acidified with 1 N HCl. The mixture is diluted with water and extracted with EtOAc. The organic layer is dried (Na₂SO₄), and the solvent is removed in vacuo to afford 0.060 g (100%) of the title compound. ¹H NMR (400 MHz, CDCl₃); MS (ES⁺) m/z mass calcd for C₃₀H₃₂O₆ 488, found 489 (M+1, 100%).

### EXAMPLE 148

### 2-Methyl-2-{4-[3-(3-phenyl-7-propyl-benzo[d]isoxazol-6-yloxy)-hexyloxy]-phenoxy}-propionic acid

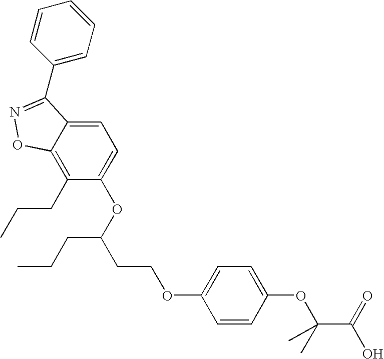

### Step A

### (2,4-Dihydroxy-3-propyl-phenyl)-phenyl-methanone oxime

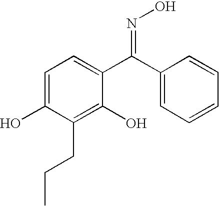

A mixture of (2,4-dihydroxy-3-propyl-phenyl)-phenyl-methanone (1.97 g, 7.69 mmol), hydroxylamine hydrochloride (3.52 g, 50.6 mmol) and sodium acetate (4.16 g, 50.6 mmol) in methanol (20 mL) is heated to reflux and stirred for 24 hours under N₂. The reaction is cooled and diluted with isopropylacetate and extracted with water and brine. The organic layer is dried (Na₂SO₄) and the solvent is removed in vacuo to afford 2.09 g (100%) of the title compound that is utilized without purification. R_{f}=0.45 (1/1 hexanes/acetone). ¹H NMR (400 MHz, CDCl₃); MS (ES⁺) m/z mass calcd for C₁₆H₁₇O₃N 271, found 272 (M+1, 100%).

### Step B

### 3-Phenyl-7-propyl-benzo[d]isoxazol-6-ol

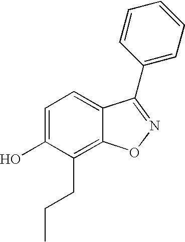

A solution of (2,4-dihydroxy-3-propyl-phenyl)-phenyl-methanone oxime (2.09 g, 7.69 mmol) in acetic anhydride (22 mL) is stirred at rt for 17 hours under N₂. The solvent is removed from the mixture in vacuo to afford a solid that is dissolved in isopropylacetate and extracted with water. The organic layer is dried (MgSO₄) and the solvent is removed in vacuo to afford 2.37 g of a residue that is dissolved in pyridine (24 mL) and heated to reflux and stirred for 8 hours under N₂. The mixture is cooled to 0° C. and quenched with 1 N HCl (200 mL). The mixture is diluted with EtOAc and extracted with water and additional 1 N HCl (200 mL). The organic layer is dried (Na₂SO₄), and the solvent is removed in vacuo to afford crude product that is absorbed on silica gel and column purified using 6/1 hexanes/acetone to afford 1.34 g (69%) of the title compound. R_{f}=0.25 (2/1 hexanes/acetone). ¹H NMR (400 MHz, CDCl₃); MS (ES⁺) m/z mass calcd for C₁₆H₁₅O₂N 253, found 254 (M+1, 100%).

### Step C

### 3-(3-Phenyl-7-propyl-benzo[d]isoxazol-6-yloxy)-hexan-1-ol

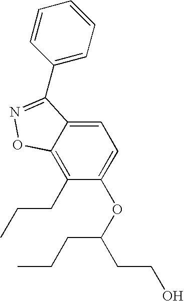

A mixture of 3-phenyl-7-propyl-benzo[d]isoxazol-6-ol (0.50 g, 1.97 mmol), 3-bromo-hexan-1-ol (0.790 g, 4.36 mmol) (Example 101, Step C) and Cs₂CO₃ (1.60 g, 4.91 mmol) in dry DMF (20 mL) is heated to 50° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford crude product that is purified by column chromatography using 98/2 CH₂Cl₂/ACN to afford 0.233 g (33%) of the title compound. R_{f}=0.26 (98/2 CH₂Cl₂/ACN). ¹H NMR (400 MHz, CDCl₃); MS (ES⁺) m/z mass calcd for C₂₂H₂₇O₃N 353, found 354 (M+1, 100%).

### Step D

### Methanesulfonic acid 3-(3-phenyl-7-propyl-benzo[d]isoxazol-6-yloxy)-hexyl ester

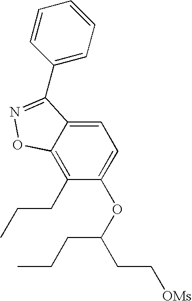

A 0° C. solution of 3-(3-phenyl-7-propyl-benzo[d]isoxazol-6-yloxy)-hexan-1-ol (0.240 g, 0.679 mmol) and TEA (0.103 g, 1.02 mmol) in CH₂Cl₂ (15 mL) is treated with MsCl (0.093 g, 0.814 mmol), and the reaction stirred for 1.5 hours at 0° C. under N₂. The reaction is quenched with 1 N HCl (20 mL) and diluted with more CH₂Cl₂ and extracted with water. The organic layer is dried (Na₂SO₄), and the solvent is removed in vacuo to afford 0.294 g (100%) of the title compound that is utilized without purification. ¹H NMR (400 MHz, CDCl₃); MS (ES⁺) m/z mass calcd for C₂₃H₂₉O₅SN 431, found 432 (M+1, 100%).

### Step E

### 2-Methyl-2-{4-[3-(3-phenyl-7-propyl-benzo[d]isoxazol-6-yloxy)-hexyloxy]-phenoxy}-propionic acid ethyl ester

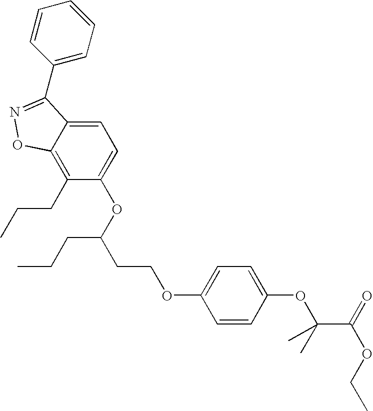

A mixture of (2-(4-hydroxy-phenoxy)-2-methyl-propionic acid ethyl ester (0.049 g, 0.219 mmol), methanesulfonic acid 3-(3-phenyl-7-propyl-benzo[d]isoxazol-6-yloxy)-hexyl ester (0.095 g, 0.220 mmol) and Cs₂CO₃ (0.086 g, 0.264 mmol) in dry DMF (7 mL) is heated to 50° C. and stirred for 17 hours under N₂. The reaction is cooled, quenched with 1 N HCl (15 mL), diluted with Et₂O and extracted with water. The organic layer is dried (Na₂SO₄) and the solvent is removed in vacuo to afford crude ester that is purified with column chromatography using 7/1 hexanes/acetone to afford 0.064 g (52%) of the title compound. R_{f}=0.39 (2/1 hexanes/acetone). ¹H NMR (400 MHz, CDCl₃); MS (ES⁺) m/z mass calcd for C₃₄H₄₁O₆N 559, found 560 (M+1, 100%).

### Step F

### 2-Methyl-2-{4-[3-(3-phenyl-7-propyl-benzo[d]isoxazol-6-yloxy)-hexyloxy]-phenoxy}-propionic acid

A solution of 2-methyl-2-{4-[3-(3-phenyl-7-propyl-benzo[d]isoxazol-6-yloxy)-hexyloxy]-phenoxy}-propionic acid ethyl ester (0.064 g, 0.114 mmol) in ethanol (6 mL) is treated with 5 N NaOH (0.50 mL) and stirred at rt until saponification is completed. The solvent is removed in vacuo to afford a residue that is acidified with 1 N HCl. The mixture is diluted with water and extracted with EtOAc. The organic layer is dried (Na₂SO₄) and the solvent is removed in vacuo to afford 0.048 g (79%) of the title compound. ¹H NMR (400 MHz, CDCl₃); MS (ES⁺) m/z mass calcd for C₃₂H₃₇O₆N 531, found 532 (M+1, 100%).

### EXAMPLE 149

### {4-[3-(3-Phenyl-7-propyl-benzo[d]isoxazol-6-yloxy)-hexylsulfanyl]-phenoxy}-acetic acid

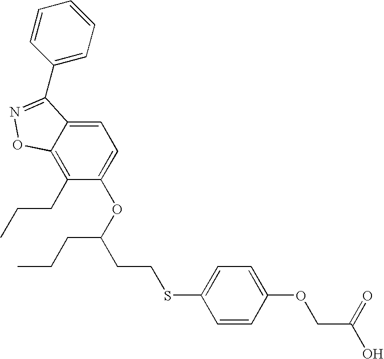

The title compound is prepared according to the procedure described in Example 148 by utilizing (4-mercapto-2-methyl-phenoxy)-acetic acid ethyl ester and methanesulfonic acid 3-(3-phenyl-7-propyl-benzo[d]isoxazol-6-yloxy)-hexyl ester to afford 0.080 g (64%). ¹H NMR (400 MHz, CDCl₃); HRMS (ES⁺) m/z exact mass calcd for C₃₁H₃₆O₅NS 534.2314, found 534.2308.

### EXAMPLE 150

### (2-Methyl-4-{1-[2-(3-phenyl-7-propyl-benzo[d]isoxazol-6-yloxy)-ethyl]-butylsulfanyl}-phenoxy)-acetic acid

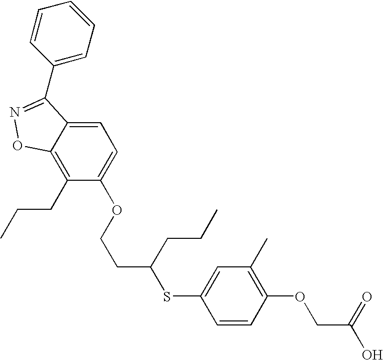

### Step A

### {4-[1-(2-Hydroxy-ethyl)-butylsulfanyl]-phenoxy}-acetic acid ethyl ester

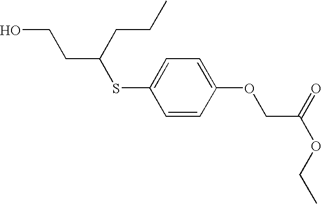

A mixture of (4-mercapto-2-methyl-phenoxy)-acetic acid ethyl ester (0.41 g, 1.81 mmol), 3-bromo-hexan-1-ol (0.360 g, 1.99 mmol) (Example 101, Step C) and Cs₂CO₃ (0.89 g, 2.73 mmol) in dry DMF (12 mL) is purged with N₂ and then heated to 50° C. and stirred for 17 hours under N₂. The reaction is cooled and acidified with 1 N HCl (20 mL). The mixture is diluted with water and extracted with Et₂O. The organic layer is dried (Na₂SO₄) and the solvent is removed in vacuo to afford crude product that is purified by column chromatography using 7/1 hexanes/acetone to afford 0.387 g (66%) of the title compound. R_{f}=0.19 (2/1 hexanes/acetone). ¹H NMR (400 MHz, CDCl₃); MS (ES⁺) m/z mass calcd for C₁₇H₂₆O₄S 326, found 327 (M+1, 100%).

### Step B

### {4-[1-(2-Methanesulfonyloxy-ethyl)-butylsulfanyl]-phenoxy}-acetic acid ethyl ester

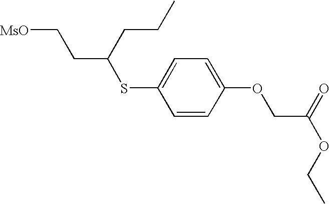

A 0° C. solution of {4-[1-(2-hydroxy-ethyl)-butylsulfanyl]-phenoxy}-acetic acid ethyl ester (0.387 g, 1.19 mmol) and TEA (0.180 g, 1.78 mmol) in CH₂Cl₂ (20 mL) is treated with MsCl (0.163 g, 1.42 mmol), and the mixture is stirred for 1.5 hours at 0° C. under N₂. The mixture is quenched with 1 N HCl (15 mL) and diluted with more CH₂Cl₂ and extracted with water. The organic layer is dried (Na₂SO₄) and the solvent is removed in vacuo to afford 0.500 g (100%) of the title compound that is utilized without purification. ¹H NMR (400 MHz, CDCl₃); MS (ES⁺) m/z mass calcd for C₁₈H₂₈O₆S₂ 404, found 405 (M+1, 100%).

### Step C

### (2-Methyl-4-{1-[2-(3-phenyl-7-propyl-benzo[d]isoxazol-6-yloxy)-ethyl]-butylsulfanyl}-phenoxy)-acetic acid ethyl ester

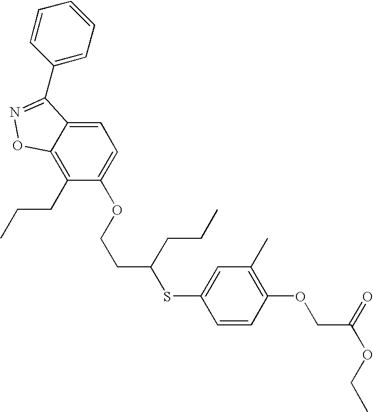

A mixture of 3-phenyl-7-propyl-benzo[d]isoxazol-6-ol (0.051 g, 0.201 mmol) (Example 147, Step B), {4-[1-(2-methanesulfonyloxy-ethyl)-butylsulfanyl]phenoxy}-acetic acid ethyl ester (0.081 g, 0.200 mmol) and Cs₂CO₃ (0.078 g, 0.239 mmol) in dry DMF (6 mL) is heated to 50° C. and stirred for 17 hours under N₂. The mixture is cooled, quenched with 1 N HCl (15 mL), diluted with Et₂O and extracted with water. The organic layer is dried (Na₂SO₄), and the solvent is removed in vacuo to afford crude ester that is purified with column chromatography using 7/1 hexanes/acetone to afford 0.063 g (56%) of the title compound. R_{f}=0.21 (2/1 hexanes/acetone). ¹H NMR (400 MHz, CDCl₃); MS (ES⁺) m/z mass calcd for C₃₃H₃₉O₅SN 561, found 562 (M+1, 100%).

### Step D

### (2-Methyl-4-{1-[2-(3-phenyl-7-propyl-benzo[d]isoxazol-6-yloxy)-ethyl]-butylsulfanyl}-phenoxy)-acetic acid

A solution of (2-methyl-4-{1-[2-(3-phenyl-7-propyl-benzo[d]isoxazol-6-yloxy)-ethyl]-butylsulfanyl}-phenoxy)-acetic acid ethyl ester (0.063 g, 0.112 mmol) in ethanol (6 mL) is treated with 5 N NaOH (0.50 mL) and stirred at rt until saponification is completed. The solvent is removed in vacuo to afford a residue that is acidified with 1N HCl. The mixture is diluted with water and extracted with EtOAc. The organic layer is dried (Na₂SO₄) and the solvent is removed in vacuo to afford 0.059 g (99%) of the title compound. ¹H NMR (400 MHz, CDCl₃); HRMS (ES⁺) m/z exact mass calcd for C₃₁H₃₅O₅SN 534.2314, found 534.2311.

### EXAMPLE 151

### (R)-3-{4-[3-(5-Chloro-pyridin-2-yloxy)-butoxy]-2-methyl-phenyl}-propionic acid

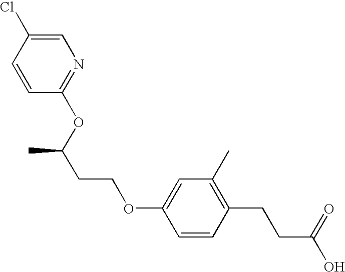

The title compound is prepared by reacting the compound of (S)-3-[4-(3-methanesulfonyloxy-butoxy)-2-methyl-phenyl]-propionic acid methyl ester with 5-chloro-pyridin-2-ol as in Exampled 107 to afford 0.044 g (37%). ¹H NMR (400 MHz, CDCl₃); HRMS (ES⁺) m/z mass calcd for C₁₉H₂₃NO₄Cl 364.1316, found 364.1311.

### EXAMPLE 152

### (R)-3-{4-[3-(4-Chloro-phenoxy)-butoxy]-2-methyl-phenyl}-propionic acid

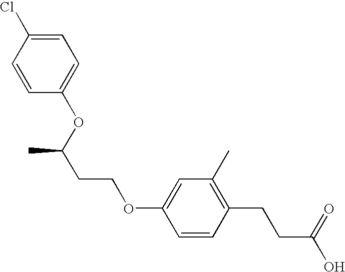

The title compound is prepared by reacting the compound of (S)-3-[4-(3-methanesulfonyloxy-butoxy)-2-methyl-phenyl]-propionic acid methyl ester with 4-chlorophenol as in Example 107 to afford 0.012 g (12%). ¹H NMR (400 MHz, CDCl₃); HRMS (ES⁺) m/z mass calcd for C₂₀H₂₂O₄Cl 361.1207, found 361.1204.

### EXAMPLE 153

### (R)-3-{4-[3-(4-Chloro-2-phenoxy-phenoxy)-butoxy]-2-methyl-phenyl}-propionic acid

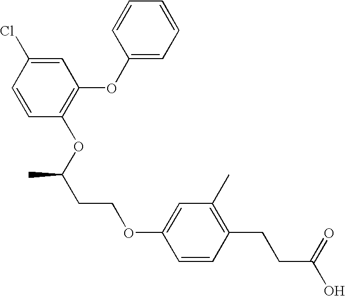

### Step A

### 4-Chloro-2-phenoxy-1-methoxy-benzene

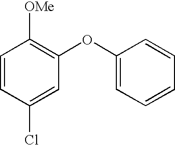

A mixture of 2-bromo-4-chloro-1-methoxy-benzene (8.0 g, 36.1 mmol), phenol (6.80 g, 72.2 mmol), cesium carbonate (23.54 g, 72.2 mmol), copper (I) chloride (1.79 g, 18.1 mmol) and 2,2,6,6-tetramethyl-3,5-heptanedione (1.66 g, 9.00 mmol) in dry 1-methyl-2-pyrrolidinone (80 mL) is heated to 120° C. for 20 hours under N₂. The reaction is cooled, filtered and the filtrate quenched with 1 N HCl (50 mL). The filtrate is diluted with Et₂O and extracted with water. The organic layer is dried (Na₂SO₄), and the solvent is removed in vacuo to afford crude product that is absorbed on silica gel and purified by flash chromatography using 9/1 hexanes/EtOAc to afford 7.42 g (88%) of the title compound. R_{f}=0.37 (4/1 hexanes/EtOAc).

### Step B

### 4-Chloro-2-phenoxy-phenol

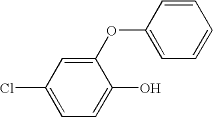

A −40° C. solution of 4-chloro-2-phenoxy-1-methoxy-benzene (7.16 g, 30.5 mmol) in dry CH₂Cl₂ (70 mL) is treated dropwise with borontribromide (22.9 g, 91.5 mmol) and then warmed to 0° C. and stirred for 3 h under N₂. The reaction is diluted with Et₂O and quenched with water. The organic layer is dried (Na₂SO₄), and the solvent is removed in vacuo to afford 7.11 g (100%) of the title compound. R_{f}=0.30 (4/1 hexanes/acetone). ¹H NMR (400 MHz, CDCl₃); MS (ES⁻) m/z mass calcd for C₁₂H₉O₂Cl 220, found 219 (M−1, 100%).

### Step C

### (R)-3-{4-[3-(4-Chloro-2-phenoxy-phenoxy)-butoxy]-2-methyl-phenyl}-propionic acid

(S)-3-[4-(3-methanesulfonyloxy-butoxy)-2-methyl-phenyl]-propionic acid methyl ester is reacted with 4-chloro-2-phenoxy-phenol as in Example 108 to afford 0.342 g (61%) of the title compound. ¹H NMR (400 MHz, CDCl₃); HRMS (ES⁺) m/z mass calcd for C₂₆H₃₁NO₅C 472.1891, found 472.1909 (M+NH₄).

### EXAMPLE 154

### (R)-3-{2-Methyl-4-[3-(2-phenoxy-4-trifluoromethyl-phenoxy)-butoxy]-phenyl}-propionic acid

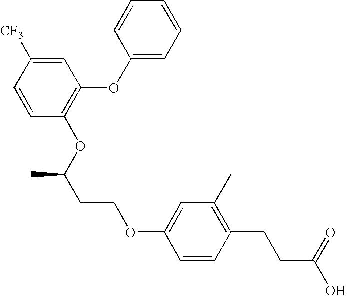

### Step A

### (R)-3-{4-[3-(2-Bromo-4-trifluoromethyl-phenoxy)-butoxy]-2-methyl-phenyl}-propionic acid methyl ester

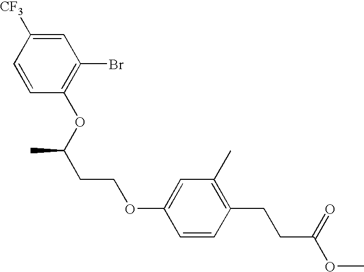

A mixture of 2-bromo-4-trifluoromethyl-phenol (0.105 g, 0.436 mmol), (S)-3-[4-(3-methanesulfonyloxy-butoxy)-2-methyl-phenyl]-propionic acid methyl ester (0.165 g, 0.479 mmol) and Cs₂CO₃ (0.184 g, 0.565 mmol) in dry DMF (7 mL) is heated to 60° C. and stirred for 17 hours under N₂. The reaction is cooled and acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using 8/1 hexanes/EtOAc to afford 0.157 g (74%) of the title compound. R_{f}=0.27 (4/1 hexanes/EtOAc). ¹H NMR (400 MHz, CDCl₃); MS (ES⁺) m/z mass calcd for C₂₂H₂₄O₄F₃Br 489, found 506 and 508 (M+17 and M+19, 100%).

### Step B

### (R)-3-{2-Methyl-4-[3-(2-phenoxy-4-trifluoromethyl-phenoxy)-butoxy]-phenyl}-propionic acid

A mixture of (R)-3-{4-[3-(2-bromo-4-trifluoromethyl-phenoxy)-butoxy]-2-methyl-phenyl}-propionic acid methyl ester (0.157 g, 0.321 mmol), phenol (0.060 g, 0.638 mmol), cesium carbonate (0.209 g, 0.642 mmol), copper (I) chloride (0.032 g, 0.323 mmol) and 2,2,6,6-tetramethyl-3,5-heptanedione (0.059 g, 0.320 mmol) in dry 1-methyl-2-pyrrolidinone (7 mL) is heated to 130° C. for 17 hours under N₂. The reaction is cooled and then quenched with 1 N HCl (10 mL). The mixture is diluted with Et₂O and extracted with water. The organic layer is dried (Na₂SO₄), and the solvent is removed in vacuo to afford crude product that is taken up in MeOH (5 mL) treated with 5 N NaOH (2 mL). After stirring at rt until saponification is completed, the solvent is removed in vacuo, and the residue is acidified with 1 N HCl. The mixture is extracted with EtOAc to give 0.420 g of crude acid that is purified by preparative HPLC to afford 0.065 g (41%) of the title compound. ¹H NMR (400 MHz, CDCl₃); HRMS (ES⁺) m/z mass calcd for C₂₇H₃₁NO₅F₃ 506.2154, found 506.2168 (M+NH₄).

### EXAMPLE 155

### (R)-3-{2-Methyl-4-[3-(2-phenoxy-4-trifluoromethoxy-phenoxy)-butoxy]-phenyl}-propionic acid

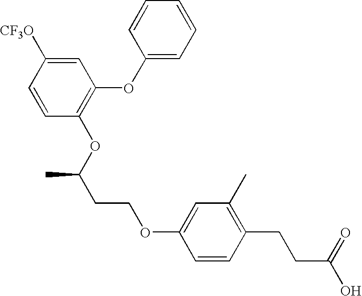

The title compound is prepared by reacting the compound of (R)3-{4-[3-(2-Bromo-4-trifluoromethoxy-phenoxy)-butoxy]-2-methyl-phenyl}-propionic acid methyl ester with phenol as in Example 154 to afford 0.030 g (11%). ¹H NMR (400 MHz, CDCl₃); HRMS (ES⁺) m/z mass calcd for C₂₇H₃₁NO₆F₃ 522.2103, found 522.2098 (M+NH₄).

### EXAMPLE 156

### (R)-3-{2-Methyl-4-[3-(4-methyl-2-phenoxy-phenoxy)-butoxy]-phenyl}-propionic acid

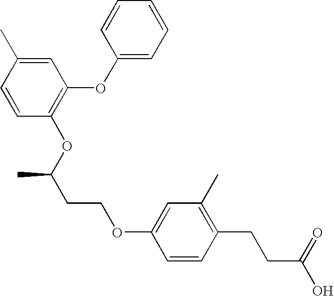

The title compound is prepared by reacting compound of (R)-3-{4-[3-(2-bromo-4-methyl-phenoxy)-butoxy]-2-methyl-phenyl}-propionic acid methyl ester with phenol as in Example 154 to afford 0.031 g (19%). ¹H NMR (400 MHz, CDCl₃); HRMS (ES⁺) m/z mass calcd for C₂₇H₃₁O₅ 435.2171, found 435.2181 (M+1).

### EXAMPLE 157

### (R)-{4-[3-(4-Chloro-2-phenoxy-phenoxy)-butoxy]-2-methyl-phenylsulfanyl}-acetic acid

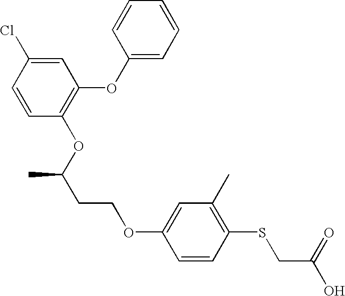

The title compound is prepared by reacting the compound of (S)-[4-(3-methanesulfonyloxy-butoxy)-2-methyl-phenylsulfanyl]-acetic acid ethyl ester with 4-chloro-2-phenoxy-phenol as in Example 108 to afford 0.056 g (55%). ¹H NMR (400 MHz, CDCl₃); HRMS (ES⁺) m/z mass calcd for C₂₅H₂₉NO₅SCl 490.1455, found 490.1447 (M+NH₄).

### EXAMPLE 158

### 3-{4-[3-(4-Chloro-2-phenoxy-phenoxy)-propoxy]-2-methyl-phenyl}-propionic acid

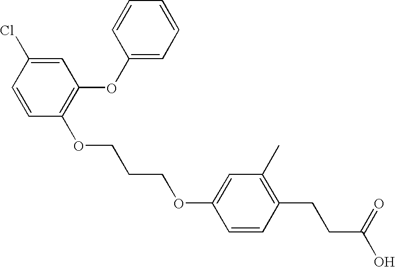

The title compound is prepared by reacting the compound of 3-[4-(3-methanesulfonyloxy-propoxy)-2-methyl-phenyl]-propionic acid methyl ester with 4-chloro-2-phenoxy-phenol as in Example 132 to afford 0.107 g (63%). ¹H NMR (400 MHz, CDCl₃); HRMS (ES⁺) m/z mass calcd for C₂₅H₂₉NO₅Cl 458.1734, found 458.1735 (M+NH₄).

### EXAMPLE 159

### (R)-3-{2-Methyl-4-[3-(1-phenoxy-naphthalen-2-yloxy)-butoxy]-phenyl}-propionic acid

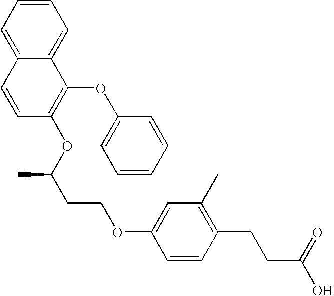

The title compound is prepared by reacting the compound of (S)-3-[4-(3-methanesulfonyloxy-butoxy)-2-methyl-phenyl]-propionic acid methyl ester with 1-phenoxy-naphthalen-2-ol as in Example 108 to afford 0.075 g (59%). ¹H NMR (400 MHz, CDCl₃); HRMS (ES⁺) m/z mass calcd for C₃₀H₃₄O₅N 488.2437, found 488.2431 (M+NH₄).

### EXAMPLE 160

### (R)-3-{4-[3-(2-Benzofuran-2-yl-4-chloro-phenoxy)-butoxy]-2-methyl-phenyl}-propionic acid

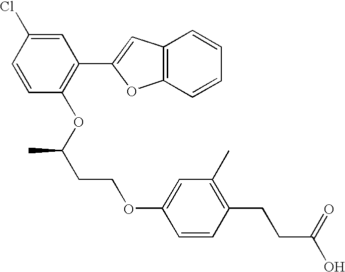

### Step A

### (R)-3-{4-[3-(2-Benzofuran-2-yl-4-chloro-phenoxy)-butoxy]-2-methyl-phenyl}-propionic acid methyl ester

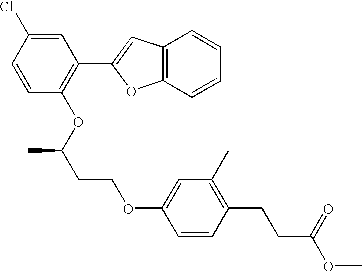

A mixture of benzo[B]furan-2-boronic acid (0.084 g, 0.519 mmol), (R)-3-{4-[3-(2-bromo-4-chloro-phenoxy)-butoxy]-2-methyl-phenyl}-propionic acid methyl ester (0.118 g, 0.259 mmol) and CsF (0.098 g, 0.645 mmol) in dry 1,4-dioxane (6 mL) is purged with N₂ and then 1,1′-bis(diphenylphospino)ferrocene palladium-(II)chloride complex with CH₂Cl₂ (0.028 g, 0.0383 mmol) is added. The mixture is heated to 80° C. and stirred for 10 hours under N₂. The reaction is cooled, and the crude product is absorbed on silica gel and purified by column chromatography using 9/1 hexanes/EtOAc to afford 0.029 g (23%) of the title compound. R_{f}=0.21 (4/1 hexanes/EtOAc). ¹H NMR (400 MHz, CDCl₃); MS (ES⁺) m/z mass calcd for C₂₂H₂₄O₄F₃Br 489, found 506 and 508 (M+17 and M+19, 100%).

### Step B

### (R)-3-{4-[3-(2-Benzofuran-2-yl-4-chloro-phenoxy)-butoxy]-2-methyl-phenyl}-propionic acid

A solution of (R)-3-{4-[3-(2-benzofuran-2-yl-4-chloro-phenoxy)-butoxy]-2-methyl-phenyl}-propionic acid methyl ester (0.029, 0.0588 mmol) in methanol (6 mL) is treated with 5 N NaOH (1.5 mL). The mixture is heated to reflux and stirred until saponification is completed. The solvent is removed in vacuo to afford a residue that is acidified with 1 N HCl. The mixture is diluted with water and extracted with EtOAc. The organic layer is dried (Na₂SO₄), and the solvent is removed in vacuo to afford 0.017 g (61%) of the title compound. ¹H NMR (400 MHz, CDCl₃); HRMS (ES⁺) m/z exact mass calcd for C₂₈H₂₈O₅Cl 479.1625, found 479.1631 (M+1, 100%).

### EXAMPLE 161

### (R)-3-{4-[3-(2-Benzo[b]thiophen-3-yl-4-chloro-phenoxy)-butoxy]-2-methyl-phenyl}-propionic acid

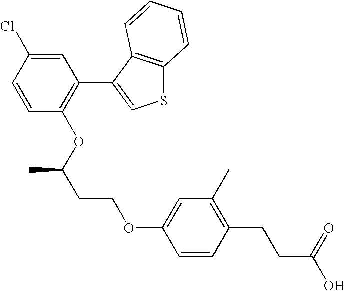

The title compound is prepared by reacting the compound of (R)-3-{4-[3-(2-bromo-4-chloro-phenoxy)-butoxy]-2-methyl-phenyl}-propionic acid methyl ester with benzothiophene-3-boronic acid as in Example 161 to afford 0.087 g (53%). ¹H NMR (400 MHz, CDCl₃); HRMS (ES⁺) m/z mass calcd for C₂₈H₃₁O₄NSCl 512.1662, found 512.1674 (M+NH₄).

### EXAMPLE 162

### (R)-3-{4-[3-(4-Chloro-2-pyridin-3-yl-phenoxy)-butoxy]-2-methyl-phenyl}-propionic acid

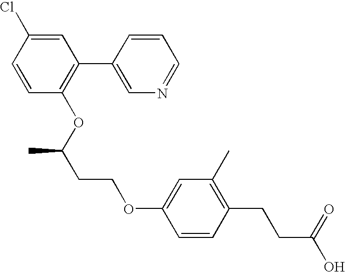

The title compound is prepared by reacting the compound of (R)-3-{4-[3-(2-bromo-4-chloro-phenoxy)-butoxy]-2-methyl-phenyl}-propionic acid methyl ester with 3-pyridine boronic acid as in Example 160 to afford 0.018 g (21%). ¹H NMR (400 MHz, CDCl₃); HRMS (ES⁺) m/z mass calcd for C₂₅H₂₇O₄NCl 440.1629, found 440.1607 (M+NH₄).

### EXAMPLE 163

### (R)-3-{4-[3-(4-Chloro-2-phenoxy-phenoxy)-butoxy]-phenyl}-2,2-difluoro-propionic acid

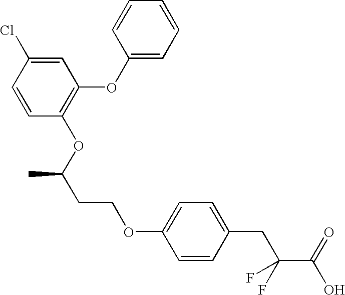

The title compound is prepared by reacting the compound of (R)-methanesulfonic acid 3-(4-chloro-2-phenoxy-phenoxy)-butyl ester with 2,2-difluoro-3-(4-hydroxy-phenyl)-propionic acid ethyl ester as in Example 63 to afford 0.058 g (42%) of the title compound. ¹H NMR (400 MHz, CDCl₃); MS (ES⁻) m/z mass calcd for C₂₅H₂₃O₅F₂Cl 476, found 475 (M−1).

### EXAMPLE 164

### (R)-3-{3-Bromo-4-[3-(4-chloro-2-phenoxy-phenoxy)-butoxy]-phenyl}-propionic acid

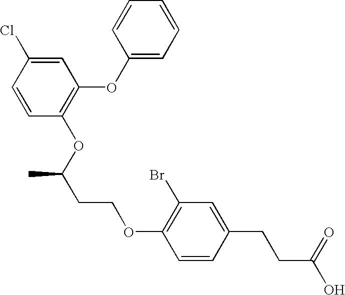

### Step A

### 3-(3-Bromo-4-hydroxy-phenyl)-propionic acid methyl ester

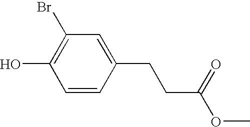

A 0° C. solution of 3-(4-hydroxy-phenyl)-propionic acid methyl ester (3.0 g, 16.6 mmol) in CH₂Cl₂ (15 mL) is treated with bromine (2.66 g, 16.7 mmol). The mixture is stirred at 0° C. for 20 minutes, warmed to rt and stirred under N₂. The reaction is diluted with water and extracted with CH₂Cl₂. The organic layer is dried (Na₂SO₄), and the solvent is removed in vacuo to afford crude product that is absorbed on silica gel and purified by column chromatography using 99/1 CH₂Cl₂/ACN to afford 3.58 g (83%) of the title compound. R_{f}=0.37 (98/2 CH₂Cl₂/ACN). ¹H NMR (400 MHz, CDCl₃); MS (ES⁻) m/z mass calcd for C₁₀H₁₁O₃Br 258, found 257 NS 259 (M−1 and M+1).

### Step B

### (R)-3-{3-Bromo-4-[3-(4-chloro-2-phenoxy-phenoxy)-butoxy]-phenyl}-propionic acid

The compound of (R)-methanesulfonic acid 3-(4-chloro-2-phenoxy-phenoxy)-butyl ester is reacted with 3-(3-bromo-4-hydroxy-phenyl)-propionic acid methyl ester as in Example 63 to afford 0.060 g (18%) of the title compound. ¹H NMR (400 MHz, CDCl₃); HRMS (ES⁺) m/z mass calcd for C₂₅H₂₈NO₅ClBr 536.0839, found 536.0830 (M+NH₄).

### EXAMPLE 165

### (R)-3-{4-[3-(4-Chloro-2-phenoxy-phenoxy)-butoxy]-3-methyl-phenyl}-propionic acid

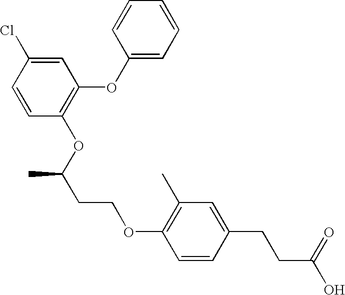

The title compound is prepared by reacting the compound of (R)-3-{3-bromo-4-[3-(4-chloro-2-phenoxy-phenoxy)-butoxy]-phenyl}-propionic acid methyl ester with methyl boronic acid as in Example 160 to afford 0.150 g (90%). ¹H NMR (400 MHz, CDCl₃); HRMS (ES⁺) m/z mass calcd for C₂₆H₃₁NO₅Cl 472.1891, found 472.1881 (M+NH₄).

### EXAMPLE 166

### (R)-3-{4-[3-(4-Chloro-2-phenoxy-phenoxy)-butoxy]-3,5-dimethyl-phenyl}-propionic acid

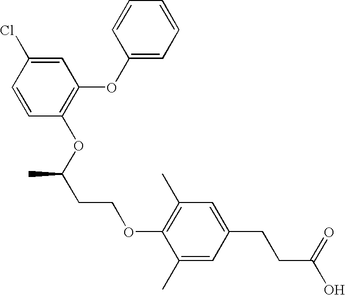

The title compound is prepared by reacting the compound of (R)-methanesulfonic acid 3-(4-chloro-2-phenoxy-phenoxy)-butyl ester with 3-(4-hydroxy-3,5-dimethyl-phenyl)-propionic acid methyl ester as in Example 63 to afford 0.095 g (69%). ¹H NMR (400 MHz, CDCl₃); HRMS (ES⁺) m/z mass calcd for C₂₇H₃₃NO₅Cl 486.2047, found 486.2051 (M+NH₄).

### EXAMPLE 167

### (R)-{3-Bromo-4-[3-(4-chloro-2-phenoxy-phenoxy)-butoxy]-phenyl}-acetic acid

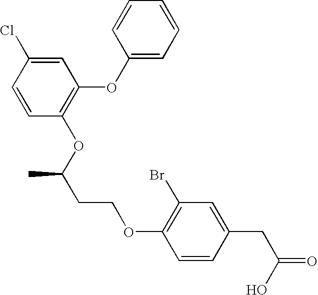

The title compound is prepared by reacting the compound of (R)-methanesulfonic acid 3-(4-chloro-2-phenoxy-phenoxy)-butyl ester with (3-bromo-4-hydroxy-phenyl)-acetic acid methyl ester as in Example 63 to afford 0.050 g (21%). ¹H NMR (400 MHz, CDCl₃); HRMS (ES⁺) m/z mass calcd for C₂₄H₂₆NO₅ClBr 522.0683, found 522.0653 (M+NH₄).

### EXAMPLE 168

### (R)-3-{2-Methyl-4-[3-(4-phenoxy-naphthalen-2-yloxy)-butoxy]-phenyl}-propionic acid

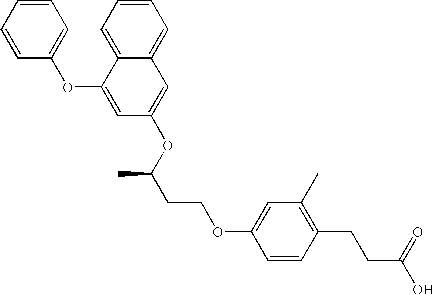

The title compound is prepared by reacting the compound of (S)-3-[4-(3-methanesulfonyloxy-butoxy)-2-methyl-phenyl]-propionic acid methyl ester with 4-phenoxy-naphthalen-2-ol as in Example 108 to afford 0.076 g (64%). ¹H NMR (400 MHz, CDCl₃); HRMS (ES⁺) m/z mass calcd for C₃₀H₃₁O₅ 471.2171, found 471.2166 (M+1).

### EXAMPLE 169

### (R)-3-{4-[3-(4-Bromo-2-trifluoromethoxy-phenoxy)-butoxy]-2-methyl-phenyl}-propionic acid

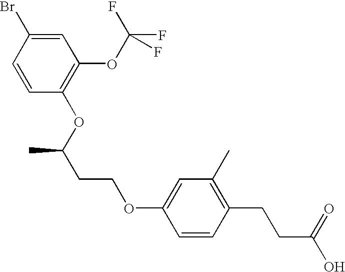

The title compound is prepared by reacting the compound of (S)-3-[4-(3-methanesulfonyloxy-butoxy)-2-methyl-phenyl]-propionic acid methyl ester with 4-bromo-2-trifluoromethoxy-phenol as in Example 108 to afford 0.033 g (23%). ¹H NMR (400 MHz, CDCl₃); HRMS (ES⁺) m/z mass calcd for C₂₁H₂₆NO₅F₃Br 508.0946, found 508.0942 (M+1).

### EXAMPLE 170

### (R)-3-{4-[3-(4-Ethyl-2-trifluoromethoxy-phenoxy)-butoxy]-2-methyl-phenyl}-propionic acid

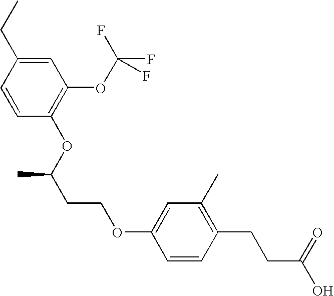

The title compound is prepared by reacting the compound of (R)-3-{4-[3-(4-bromo-2-trifluoromethoxy-phenoxy)-butoxy]-2-methyl-phenyl}-propionic acid methyl ester (Example 169) with ethyl boronic acid as in Example 160 to afford 0.073 g (60%) after saponification. ¹H NMR (400 MHz, CDCl₃); HRMS (ES⁺) m/z mass calcd for C₂₃H₃₁NO₅F₃ 458.2154, found 458.2160 (M+1).

### EXAMPLE 171

### (R)-{4-[3-(4-Chloro-2-phenoxy-phenoxy)-butoxy]-3-methyl-phenyl}-acetic acid

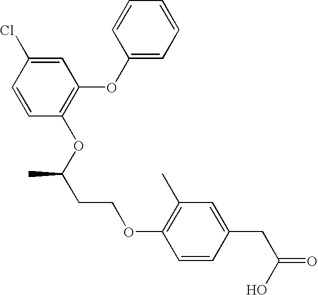

The title compound is prepared by reacting the compound of (R)-{3-bromo-4-[3-(4-chloro-2-phenoxy-phenoxy)-butoxy]-phenyl}-acetic acid methyl ester with methyl boronic acid as in Example 160 to afford 0.086 g (53%). ¹H NMR (400 MHz, CDCl₃); HRMS (ES⁺) m/z mass calcd for C₂₅H₂₉NO₅Cl 458.1734, found 458.1723 (M+NH₄).

### EXAMPLE 172

### (R)-{4-[3-(4-Chloro-2-phenoxy-phenoxy)-butoxy]-phenyl}-acetic acid

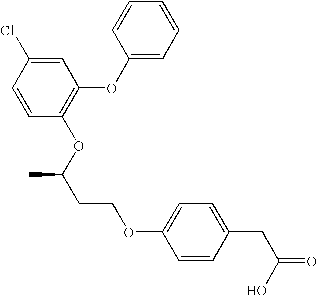

The title compound is prepared by reacting the compound of (R)-methanesulfonic acid 3-(4-chloro-2-phenoxy-phenoxy)-butyl ester with (4-hydroxy-phenyl)-acetic acid methyl ester as in Example 63 to afford 0.094 g (52%). ¹H NMR (400 MHz, CDCl₃); HRMS (ES⁺) m/z mass calcd for C₂₄H₂₇O₅NCl 444.1578, found 444.1588.

### EXAMPLE 173

### {4-[3-(4-Chloro-2-phenoxy-phenoxy)-propyl]-2-methyl-phenoxy}-acetic acid

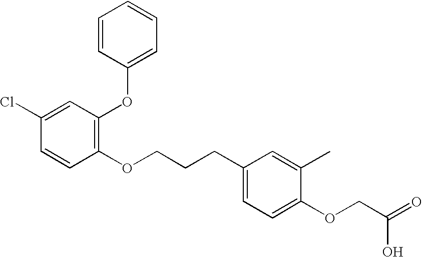

### Step A

### [4-(3-Hydroxy-propyl)-2-iodo-phenoxy]-acetic acid ethyl ester

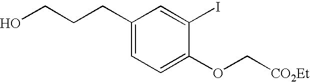

A mixture of [4-(3-hydroxy-propyl)-phenoxy]-acetic acid ethyl ester ethyl ester (0.50 g, 2.09 mmol), silver sulfate (1.31 g, 4.20 mol) and iodine (1.07 g, 4.22 mmol) in ethanol (10 mL) is stirred at rt for 17 hours under N₂. The mixture is filtered, and the solvent is removed in vacuo to afford crude product that is purified by column chromatography using 3/1 hexanes/acetone afford 0.24 g (31%) of the title compound. R_{f}=0.21 (2/1 hexanes/acetone).

### Step B

### [4-(3-Hydroxy-propyl)-2-methyl-phenoxy]-acetic acid ethyl ester

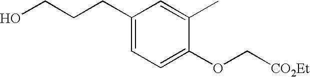

A mixture of [4-(3-hydroxy-propyl)-2-iodo-phenoxy]-acetic acid ethyl ester (0.23 g, 0.632 mmol), methylboronic acid (0.113 g, 1.89 mol) and cesium fluoride (0.34 g, 2.24 mmol) in 1,4-dioxane (4 mL) is stirred at rt and purged with N₂ for 3 minutes. The reaction is treated with 1,1′-bis(diphenylphosphino)ferrocene palladium (II) chloride, CH₂Cl₂ complex (0.040 g) and then stirred at 80° C. for 1 hour under N₂. The mixture is cooled, and the solvent is removed in vacuo to afford crude product that is absorbed on silica gel and purified by column chromatography using 3/1 hexanes/acetone afford 0.086 g (54%) of the title compound. R_{f}=0.37 (1/1 hexanes/acetone).

### Step C

### {2-Methyl-4-[3-(toluene-4-sulfonyloxy)-propyl]-phenoxy}-acetic acid ethyl ester

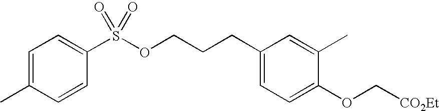

A solution of [4-(3-hydroxy-propyl)-2-methyl-phenoxy]-acetic acid ethyl ester (0.086 g, 0.341 mmol), pyridine (0.108 g, 1.36 mmol) and N,N-dimethylaminopyridine (0.012 g, 0.098 mmol) in CH₂Cl₂ (8 mL) is treated with p-toluenesulfonic anhydride (0.222 g, 0.680 mmol). and the reaction is stirred at rt for an hour under N₂. The reaction is quenched with 1 N HCl (5 mL) and diluted with more CH₂Cl₂ and extracted with water. The organic layer is dried (Na₂SO₄), and the solvent is removed in vacuo to afford crude product that is purified by column chromatography using 6/1 hexanes/acetone to afford 0.117 g (84%) of the title compound. R_{f}=0.49 (1/1 hexanes/acetone). ¹H NMR (400 MHz, CDCl₃). MS (ES⁺) m/z mass calcd for C₂₁H₂₆O₆S 406, found 424 (M+NH₄).

### Step D

### {4-[3-(4-Chloro-2-phenoxy-phenoxy)-propyl]-2-methyl-phenoxy}-acetic acid

The compound of {2-methyl-4-[3-(toluene-4-sulfonyloxy)-propyl]-phenoxy}-acetic acid ethyl ester is reacted with 4-chloro-2-phenoxy-phenol as in Example 98 to afford 0.054 g (67%) of the title compound. ¹H NMR (400 MHz, CDCl₃); HRMS (ES⁺) m/z mass calcd for C₂₄H₂₇O₅NCl 444.1578, found 444.1583.

### EXAMPLE 174

### (R)-2-{4-[3-(4-Chloro-2-phenoxy-phenoxy)-butoxy]-2-methyl-phenyl}-cyclopropanecarboxylic acid

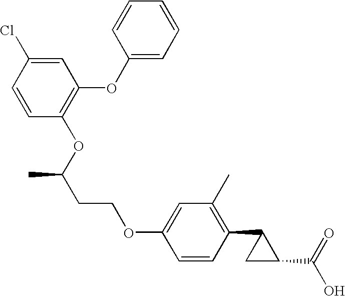

### Step A

### 2-(4-Benzyloxy-2-methyl-phenyl)-cyclopropanecarboxylic acid methyl ester

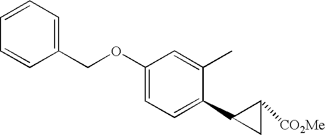

A mixture of trimethylsulfoxonium iodide (0.88 g, 4.00 mmol) in DMSO (5 mL) is treated with 1 N potassium tert-butoxide in THF (4 mL, 4.00 mmol), and the resultant mixture is stirred at rt for 20 minutes under N₂. A solution of 3-(4-benzyloxy-2-methyl-phenyl)-acrylic acid methyl ester (0.75 g, 2.65 mmol) in dry THF (6 mL) is added dropwise, and the reaction is stirred 17 h at rt. The mixture is quenched with 1N HCl (10 mL), diluted with water and extracted with Et₂O. The organic layer is dried (Na₂SO₄), and the solvent is removed in vacuo to afford crude product that is absorbed on silica gel and purified by column chromatography 7/1 hexanes/EtOAc to afford 0.076 g (10%) of the title compound. ¹H NMR (400 MHz, CDCl₃).

### Step B

### 2-(4-Hydroxy-2-methyl-phenyl)-cyclopropanecarboxylic acid methyl ester

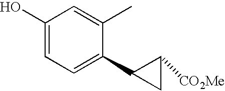

A mixture 2-(4-benzyloxy-2-methyl-phenyl)-cyclopropanecarboxylic acid methyl ester (0.076 g, 0.256 mmol) and 10% Pd/C (80 mg) in EtOAc (20 mL) is purged with N₂ and then hydrogen. The mixture is stirred under a hydrogen balloon for 2 hours at rt. The mixture is filtered through hyflo to remove the catalyst, and the solvent is removed in vacuo from the filtrate to afford 0.056 g (100%) of the title compound. MS (ES⁻) m/z mass calcd for C₁₂H₁₄O₃ 206, found 205 (M−1).

### Step C

### (R)-2-{4-[3-(4-Chloro-2-phenoxy-phenoxy)-butoxy]-2-methyl-phenyl}-cyclopropanecarboxylic acid

The compound of (R)-methanesulfonic acid 3-(4-chloro-2-phenoxy-phenoxy)-butyl ester is reacted with (2-(4-hydroxy-2-methyl-phenyl)-cyclopropanecarboxylic acid methyl ester as in Example 63 to afford 0.086 g (68%) of the title compound. ¹H NMR (400 MHz, CDCl₃); HRMS (ES⁺) m/z mass calcd for C₂₇H₃₁O₅NCl 484.1891, found 484.1883.

### EXAMPLE 175

### (R)-3-{4-[3-(4-Chloro-2-phenoxy-phenoxy)-butoxy]-2-trifluoromethyl-phenyl}-propionic acid

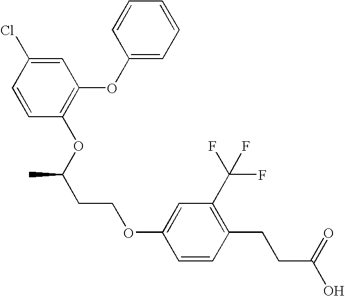

### Step A

### 1-Benzyloxy-4-bromo-3-trifluoromethyl-benzene

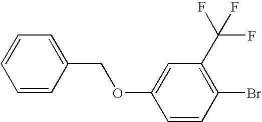

A mixture of 4-bromo-3-trifluoromethyl-phenol (10.95 g, 45.4 mmol) and 325 mesh K₂CO₃ (7.54 g, 54.6 mmol) in DMF (80 mL) is treated with benzyl bromide (8.55 g, 50.0 mmol) and stirred at 55° C. hr for 3 h under N₂. The mixture is filtered using Et₂O to rinse the solids, and the filtrate is acidified with 1 N HCl. The filtrate is diluted with more Et₂O and then extracted with twice with water and brine. The organic layer is dried (Na₂SO₄), and the solvent is removed in vacuo to afford crude product that is absorbed onto silica gel and column purified with 9/1 hexanes/EtOAc to afford 14.46 g (96%) of the title compound. R_{f}=0.47 (4/1 hexanes/acetone). ¹H NMR (400 MHz, CDCl₃).

### Step B

### 4-Benzyloxy-2-trifluoromethyl-benzaldehyde

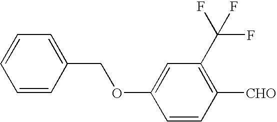

A −78° C. solution of 1-benzyloxy-4-bromo-3-trifluoromethyl-benzene (6.00 g, 18.1 mmol) in dry THF (60 mL) is treated dropwise with a 1.6 M solution of n-butyl lithium (17.0 mL, 27.1 mmol), and the reaction is stirred 10 minutes at −78° C. DMF (7.92 g, 0.108 mol) is added, and the mixture is warmed to rt and stirred. The reaction is quenched with 1 N HCl, diluted with Et₂O and extracted with water. The organic layer is dried (Na₂SO₄), and the solvent is removed in vacuo to afford crude product that is absorbed onto silica gel and column purified with 9/1 hexanes/EtOAc to afford 2.92 g (57%) of the title compound. R_{f}=0.56 (2/1 hexanes/EtOAc). ¹H NMR (400 MHz, CDCl₃); MS (ES⁺) m/z mass calcd for C₁₅H₁₁O₂F₃ 280, found 281 (M+1, 100%).

### Step C

### 3-(4-Benzyloxy-2-trifluoromethyl-phenyl)-acrylic acid ethyl ester

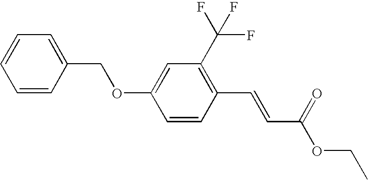

A mixture of 4-benzyloxy-2-trifluoromethyl-benzaldehyde (2.92 g, 10.4 mmol), triethyl phosphonoacetate (2.80 g, 12.5 mmol) and 325 mesh K₂CO₃ (4.32 g, 31.3 mmol) in ethanol (40 mL) is heated to reflux until starting material is gone by TLC (2/1 hexanes/EtOAc). The reaction is cooled, filtered, and the filtrate is quenched with 1 N HCl. The filtrate is diluted with water and extracted with EtOAc. The organic layer is dried (Na₂SO₄) and the solvent is removed in vacuo to afford crude product that is absorbed onto silica gel and column purified with 9/1 hexanes/EtOAc to afford 3.10 g (85%) of the title compound. R_{f}=0.40 (2/1 hexanes/EtOAc). ¹H NMR (400 MHz, CDCl₃); MS (ES⁺) m/z mass calcd for C₁₉H₁₇O₃F₃ 350, found 351 (M+1, 100%).

### Step D

### 3-(4-Hydroxy-2-trifluoromethyl-phenyl)-propionic acid ethyl ester

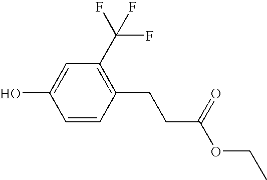

A mixture of 3-(4-benzyloxy-2-trifluoromethyl-phenyl)-acrylic acid ethyl ester (3.10 g, 8.85 mmol) and 10% palladium on carbon (2.0 g) in EtOAc (100 mL) is purged with N₂ then hydrogen, and then stirred under a hydrogen balloon for 4 h at rt. The reaction is filtered through hyflo to remove the catalyst, and the organic layer is dried (Na₂SO₄). The solvent is removed in vacuo to afford 2.46 g (100%) of the title compound. R_{f}=0.41 (2/1 hexanes/EtOAc). ¹H NMR (400 MHz, CDCl₃); MS (ES⁺) m/z mass calcd for C₁₂H₁₃O₃F₃ 262, found 261 (M−1, 100%).

### Step E

### (R)-3-{4-[3-(4-Chloro-2-phenoxy-phenoxy)-butoxy]-2-trifluoromethyl-phenyl}-propionic acid

The compound of (R)-methanesulfonic acid 3-(4-chloro-2-phenoxy-phenoxy)-butyl ester is reacted with 3-(4-hydroxy-2-trifluoromethyl-phenyl)-propionic acid ethyl ester as in Example 63 to afford 0.764 g (75%) of the title compound. ¹H NMR (400 MHz, CDCl₃); HRMS (ES⁺) m/z mass calcd for C₂₆H₂₈O₅F₃ClN 526.1608, found 526.1597 (M+NH₄).

### EXAMPLE 176

### (R)-{4-[3-(4-chloro-2-phenoxy-phenoxy)-butylsulfanyl]-2-methyl-phenoxy}-acetic acid

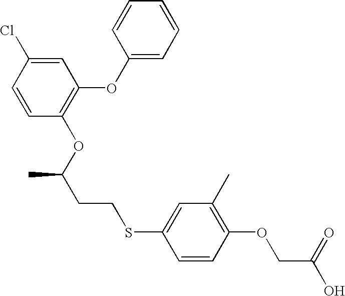

A solution of (4-mercapto-2-methyl-phenoxy)-acetic acid ethyl ester (0.219 g, 0.968 mmol) and (R)-methanesulfonic acid 3-(4-chloro-2-phenoxy-phenoxy)-butyl ester (0.30 g, 0.809 mmol) in DMF (7 mL) is purged with N₂, and then 325 mesh K₂CO₃ (0.145 g, 1.05 mmol) is added. The mixture is stirred at rt for 17 hours under N₂. The reaction is acidified with 1 N HCl (20 mL). The mixture is diluted with water and extracted with Et₂O. The organic layer is dried (Na₂SO₄), and the solvent is removed in vacuo to afford crude product that is absorbed on silica gel and purified by column chromatography using a gradient of 7/1 to 4/1 hexanes/EtOAc to afford 0.361 g (74%) of (R)-{4-[3-(4-chloro-2-phenoxy-phenoxy)-butylsulfanyl]-2-methyl-phenoxy}-acetic acid ethyl ester [R_{f}=0.29 (4/1 hexanes/EtOAc)]. The ester then is saponified to afford 0.333 g (98%) of the title compound. ¹H NMR (400 MHz, CDCl₃); HRMS (ES⁺) m/z mass calcd for C₂₅H₂₅O₅SCl 473.1189, found 473.1172 (M+1).

### EXAMPLE 177

### (R)-3-{4-[3-(4-Chloro-2-phenoxy-phenoxy)-butylsulfanyl]-2-methyl-phenyl}-propionic acid

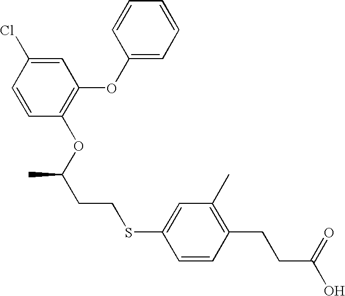

### Step A

### 3-(4-Mercapto-2-methyl-phenyl)-propionic acid methyl ester

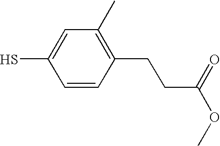

The compound of 3-(4-hydroxy-2-methyl-phenyl)-propionic acid methyl ester (5.0 g, 25.75 mmol) is dissolved into dry dioxane (100 mL) and combined with 4-dimethylamino pyridine (0.500 g, 2.6 mmol), TEA (7.0 mL, 51.5 mmol) and dimethylaminothiocarbomoyl chloride (4.5 g, 32.17 mmol). The mixture is heated to reflux under nitrogen. The reaction is monitored by TLC until phenol is completely consumed after 20 hours. After cooling to rt, the reaction is diluted with EtOAc (200 mL). Water (75 mL) is added and the two layers are separated. The organic layer is washed with brine (75 mL) then dried over anhydrous sodium sulfate. The solvent is removed, and the residue is dried under vacuum to give 3-(4-dimethylthiocarbamoyloxy-2-methyl-phenyl)-propionic acid methyl ester.

The 3-(4-dimethylthiocarbamoyloxy-2-methyl-phenyl)-propionic acid methyl ester, taken crude from the previous step, is diluted with 75 mL of tetradecane and heated to reflux under nitrogen. The reaction is monitored by TLC until all the conversion is completed after 20 h. The reaction is cooled to rt, and tetradecane is decanted away from the resulting oil. The residue is rinsed several times with hexanes. This oil is then purified using flash column chromatography to afford 5.01 g (69%) of 3-(4-dimethylcarbamoylsulfanyl-2-methyl-phenyl)-propionic acid methyl ester. This propionic acid methyl ester (5.01 g, 17.8 mmol) is diluted with methanol (30 mL) and sodium methoxide (1.7 mL of 4M in methanol, 7.23 mmol) is added. The reaction is heated to reflux under nitrogen and monitored by TLC. After complete conversion, the reaction is cooled to rt, and then neutralized with 1N HCl (7.23 mL) and diluted with EtOAc (150 mL). The two phases are separated, and the organic layer is washed with water (75 mL) and brine (75 mL). The organic layer is dried over anhydrous sodium sulfate and concentrated to yield 4.43 g crude product, which is used without further purification.

### Step B

### (R)-3-{4-[3-(4-Chloro-2-phenoxy-phenoxy)-butylsulfanyl]-2-methyl-phenyl}-propionic acid

The compound of (R)-methanesulfonic acid 3-(4-chloro-2-phenoxy-phenoxy)-butyl ester is reacted with 3-(4-mercapto-2-methyl-phenyl)-propionic acid methyl ester as in Example 176 to afford 0.329 g (86%) of the title compound. ¹H NMR (400 MHz, CDCl₃); HRMS (ES⁺) m/z mass calcd for C₂₆H₃₁O₄SClN 487.1346, found 487.1331 (M+NH₄).

### EXAMPLE 178

### Preparation of 2-Cyclopropylmethyl-4-trifluoromethyl-phenol

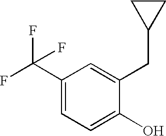

### Step A

### 1-Methoxy-4-trifluoromethyl-benzene

The compound of 4-hydroxybenzotrifluoride (15.0 g, 93 mmol) is dissolved in acetone (400 ml), and K₂CO₃ (19.3 g, 140 mmol) and MeI (17.3 mL, 280 mmol) are added. The mixture is stirred at rt overnight. The precipitate is filtered and the filtrate is concentrated, which is dissolved in EtOAc, washed with brine, dried over Na₂SO₄ and concentrated under reduced pressure. Purification by flash chromatography, eluting with EtOAc:hexane (1:5) provides the title compound (11.5 g, 70%). GC/MS: M.⁺ 176; ¹HNMR (400 MHz, CDCl₃)

### Step B

### 2-Cyclopropylmethyl-1-methoxy-4-trifluoromethyl-benzene

N,N,N′,N′-tetramethylethylenediamine (TMEDA, 6.00 mL, 40 mmol) is dissolved in THF (30 ml), and the solution is cooled to −78° C. n-BuLi (1.6 M in hexane; 25.0 mL, 40 mmol) is added slowly, and the mixture is stirred for 15 min. The compound of 1-methoxy-4-trifluoromethyl-benzene (3.48 g, 20 mmol) is added in THF (20 mL) at −78° C. and is stirred at −20° C. to −30° C. for 2 h. Cyclopropylmethylbromide (4.80 mL, 49 mmol) is added at −78° C. and stirred at −78° C. to rt overnight. The mixture is quenched with aqueous NH₄Cl, extracted with EtOAc, washed with brine, dried over Na₂SO₄ and under reduced pressure. Purification by chromatography, eluting with 5% EtOAc in hexane then 10% EtOAc in hexane provides the title compound (1.54 g, 33%). GC/MS: M.⁺ 230; ¹HNMR (400 MHz, CDCl₃)

### Step C

### 2-Cyclopropylmethyl-4-trifluoromethyl-phenol

The compound from Step B (1.54 g, 6.7 mmol) is dissolved in CH₂Cl₂ (15 mL), n-Bu₄NI (4.95 g, 13.4 mmol) is added and the mixture is cooled to −78° C. BCl₃ (1M in CH₂Cl₂, 13.4 mL, 13.4 mmol) is added slowly and mixture is stirred at 0° C. for about 0.5 h and rt for about 1.5 h. The mixture is quenched with ice/H₂O at 0° C. and stirred for 0.5 h. The mixture is extracted with CH₂Cl₂, washed with brine, dried over Na₂SO₄ and concentrated under reduced pressure. Purification by chromatography, eluting with 10% EtOAc in hexane and 15% EtOAc in hexane provides the title compound (0.91 g, 63%). Mass (ES⁻): 215 (M−H); ¹HNMR (400 MHz, CDCl₃).

### EXAMPLE 179

### Preparation of 2-Cyclohexylmethyl-4-trifluoromethyl-phenol

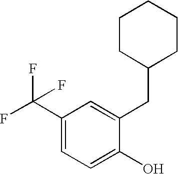

### Step A

### 2-Cyclohexylmethyl-1-methoxy-4-trifluoromethyl-benzene

TMEDA (5.1 mL, 33.6 mmol) is dissolved in THF (30 ml), and the mixture is cooled to −78° C. n-BuLi (1.6 M in hexane; 21.0 mL, 33.6 mmol) is added slowly and stirred for 15 min. The compound of 1-methoxy-4-trifluoromethyl-benzene (2.96 g, 16.8 mmol) is added in THF (20 mL) at −78° C., and the mixture is stirred at −10° C. to −30° C. for 4 h. Cyclohexylmethyl bromide (5.2 mL, 37.0 mmol) is added at −78° C. and stirred at −78° C. to rt overnight. The mixture is quenched with aqueous NH₄Cl, extracted with EtOAc, washed with brine, dried over Na₂SO₄ and concentrated under reduced pressure. Purification by chromatography, eluting with 5% EtOAc in hexane then 10% EtOAc in hexane provided the title compound (0.95 g, 21%). GC/MS: M.⁺ 272; ¹HNMR (400 MHz, CDCl₃).

### Step B

### 2-Cyclohexylmethyl-4-trifluoromethyl-phenol

The compound from Step A (0.95 g, 3.5 mmol) is dissolved in CH₂Cl₂ (30 mL), and n-Bu₄NI (3.21 g, 8.7 mmol) is added. The mixture is cooled to −78° C. and BCl₃ (1 M in CH₂Cl₂, 8.7 mL, 8.7 mmol) is added slowly. The mixture is stirred at 0° C. for about 45 min and rt for about 1.5 h. The mixture is quenched with ice/H₂O at 0° C., stirred for 0.5 h and extracted with CH₂Cl₂, which then washed with brine, dried over Na₂SO₄, and concentrated under reduced pressure. The residue is triturated with EtOAc, the precipitate is filtered, and the filtrate is concentrated. Purification by chromatography, eluting with 10% EtOAc in hexane then 15% EtOAc in hexane provides the title compound (0.74 g, 82%). MS: (ES⁻): 257 (M−H⁺); ¹HNMR (400 MHz, CDCl₃).

### EXAMPLE 180

### Preparation of 2,7-Dimethyl-3-phenyl-benzofuran-6-ol

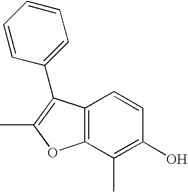

### Step A

### 6-Methoxy-2-methyl-3-phenyl-benzofuran

The compound of 6-methoxy-3-phenyl-benzofuran (5.52 g, 24.6 mmol) is dissolved in THF (80 mL), and the mixture is cooled to −78° C. and n-BuLi (1.6M in hexane; 16.6 mL, 26.5 mmol) is added slowly. The mixture is warmed to −10° C. to −20° C. and stirred for 3 h. MeI (1.65 mL, 26.5 mmol) is added and the mixture is stirred at −78° C. to rt overnight. The mixture is quenched with aqueous NH₄Cl, extracted with EtOAc, washed with brine, dried over Na₂SO₄ and concentrated. Purification by chromatography, eluting with EtOAc:hexane (1:5) provides the title compound (5.19 g, 89%). GC/MS: M.⁺ 238; ¹HNMR (400 MHz, CDCl₃).

### Step B

### 6-Methoxy-2,7-dimethyl-3-phenyl-benzofuran

TMEDA (1.66 mL, 11 mmol) is dissolved in THF (10 mL), the mixture is cooled to −78° C., and n-BuLi (1.6 M in hexane, 6.7 mL, 11 mmol) is added slowly. The mixture is stirred for 15 min and 6-methoxy-2-methyl-3-phenyl-benzofuran (1.16 g, 4.9 mmol) in THF (30 ml) is added at −78° C. and stirred at −68° C. for 1 h. MeI (0.76 mL, 12 mmol) is added at −78° C., and the mixture is stirred at −78° C. to rt for 1 h. The mixture is quenched with aqueous NH₄Cl, extracted with EtOAc, washed with brine, dried over Na₂SO₄ and concentrated under reduced pressure. Purification by chromatography, eluting with 10% CH₂Cl₂ in hexane provides the title compound (0.40 g, 33%) along with a side-product, 6-methoxy-2-ethyl-3-phenyl-benzofuran (0.49 g). GC/MS: M.⁺ 252;

¹HNMR (400 MHz, CDCl₃).

### Step C

### 2,7-Dimethyl-3-phenyl-benzofuran-6-ol

The compounds of 6-methoxy-2,7-dimethyl-3-phenyl-benzofuran (0.40 g, 1.59 mmol) and terabutylammonium iodide (1.47 g, 3.97 mmol) are dissolved in DCM (15 mL) and cool to −78° C. followed by a dropwise addition of boron trichloride solution (4.0 mL, 1.0 M in DCM, 3.97 mmol). The mixture is stirred for 0.5 hours at 0° C. and then 1.5 hours at rt. The mixture is quenched with ice water and stirred for 0.5 hours and then diluted additional water and DCM. Organic layer is separated, washed with brine, dried over sodium sulfate, and concentrated under reduced pressure. Purification by flash chromatography, eluting with 10% EtOAc in hexane then 15% EtOAc in hexane (linear gradient) provides the title compound (0.27 g, 76%). GC/MS: M.⁺ 238; ¹H NMR (400 MHz, CDCl₃).

### EXAMPLE 181

### Preparation of 4-Methyl-3-phenyl-benzofuran-6-ol

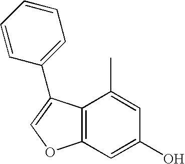

### Step A

### 2-(3-Methoxy-5-methyl-phenoxy)-1-phenyl-ethanone

A mixture of 2-bromoacetophenone (7.20 g, 36 mmol), 3-methoxy-5-methylphenol (5.00 g, 36 mmol) and K₂CO₃ (7.45 g, 54 mmol) in methyl ethyl ketone (78 mL) is heated under reflux overnight. The precipitate is filtered, and the filtrate is concentrated and partitioned between EtOAc and aqueous NaCl. Organic layer is washed with brine and dried over Na₂SO₄ and concentrated under reduced pressure. Purification by chromatography, eluting with EtOAc:hexane (1:5) provides the title compound (8.78 g, 95%). GC/MS: M.⁺ 256; ¹HNMR (400 MHz, CDCl₃).

### Step B

### 6-Methoxy-4-methyl-3-phenyl-benzofuran

Amberlyst 15 (8 g) under reflux in toluene (200 mL) is heated for 1 h with a Dean and Stark separator to remove water. The compound from Step B (8.69 g, 34 mmol) is added after cool to rt, and the mixture is heated under reflux for 3 h. The mixture is cooled to rt, the precipitate is filtered and the filtrate is concentrated. Purification by chromatography, eluting with 10% CH₂Cl₂ in hexane and then 15% CH₂Cl₂ in hexane provides the title compound (4.83 g, 60%). GC/MS: M.⁺ 238; ¹HNMR (400 MHz, CDCl₃).

### Step C

### 4-Methyl-3-phenyl-benzofuran-6-ol

The title compound is prepared according to the procedure used in Example 180, Step 3 using 6-methoxy-4-methyl-3-phenyl-benzofuran. Purification by flash chromatography, eluting with 10% EtOAc in hexane then 15% EtOAc in hexane (linear gradient) provides the title compound (0.31 g, 65%). GC/MS: M⁺ 224; ¹H NMR (400 MHz, CDCl₃).

### EXAMPLE 182

### Preparation of 4-Methyl-3-phenyl-7-propyl-benzofuran-6-ol

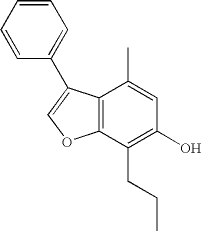

### Step A

### (6-Methoxy-4-methyl-3-phenyl-benzofuran-2-yl)-trimethyl-silane

The compound of 6-methoxy-4-methyl-3-phenyl-benzofuran (1.5 g, 6.3 mmol) is dissolved in THF (10 mL), and the mixture is cooled to −78° C. and then n-BuLi (1.6 M in hexane, 4.33 mL, 6.9 mmol) is added and stirred at −78° C. for 1 h. TMSCl (1.2 mL, 9.5 mmol) is added and stirred at −78° C. for 1 h, and then rt overnight. The mixture is quenched with aqueous NH₄Cl, extracted with EtOAc, washed with water and brine, and then dried over Na₂SO₄ and concentrated under reduced pressure. Purification by chromatography, eluting with 10% CH₂Cl₂ in hexane and then 15% CH₂Cl₂ in hexane provides the title compound (0.64 g, 33%) with a mixture of the title compound and the starting material (0.51 g). GC/MS: M.⁺ 310; ¹HNMR (400 MHz, CDCl₃.

### Step B

### (6-Methoxy-4-methyl-3-phenyl-7-propyl-benzofuran-2-yl)-trimethyl-silane

TMEDA (0.49 ml, 3.28 mmol) is dissolved in THF (10 mL), and the mixture is cooled to −78° C., and then n-BuLi (1.6 M in hexane, 2.1 mL, 3.28 mmol) is added slowly and stirred for 15 min. The compound from Step A (0.51 g, 1.64 mmol) is added in THF (15 ml) at −78° C. and warmed to −30° C. for 1.5 h followed by the addition of 1-iodopropane (0.48 mL, 4.92 mmol) at −78° C. The mixture is stirred at −78° C. to rt for 3 h and then quenched with aqueous NH₄Cl, extracted with EtOAc, washed with brine, dried over Na₂SO₄ and concentrated under reduced pressure. Purification by chromatography, eluting with 10% CH₂Cl₂ in hexane and then 15% CH₂Cl₂ in hexane provides the title compound (0.40 g, 69%). GC/MS: M.⁺ 352; ¹HNMR (400 MHz, CDCl₃).

### Step C

### 6-Methoxy-4-methyl-3-phenyl-7-propyl-benzofuran

The compound obtained from Step B (0.40 g, 1.14 mmol) is dissolved in THF(10 mL) and n-Bu₄NF (1M in THF, 1.70 mL, 1.70 mmol) is added. The mixture is stirred at rt overnight. The mixture is diluted with EtOAc, washed with brine, dried over Na₂SO₄ and concentrated under reduced pressure. Purification by chromatography, eluting with 10% CH₂Cl₂ in hexane and then 15% CH₂Cl₂ in hexane provides the title compound (0.29 g, 92%). GC/MS: M.⁺ 280; ¹HNMR (400 MHz, CDCl₃).

### Step D

### 4-Methyl-3-phenyl-7-propyl-benzofuran-6-ol

The title compound is prepared by following the procedure described in Example 180, Step 3 using 6-methoxy-4-methyl-3-phenyl-7-propyl-benzofuran. Purification by chromatography, eluting with 10% EtOAc in hexane then 15% EtOAc in hexane (linear gradient) provides the title compound (0.11 g, 38%). MS: (ES⁻) 265 (M−H); ¹H NMR (400 MHz, CDCl₃).

### EXAMPLE 183

### Preparation of 2-Methyl-3-phenyl-7-propyl-benzofuran-6-ol

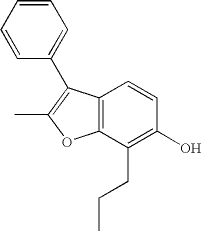

### Step A

### 2-methyl-3-phenyl-benzofuran-6-ol

A mixture of 6-methoxy-2-methyl-3-phenyl-benzofuran (Example 181, Step 1) (1.9 g, 7.97 mmol) and pyridine HCl (11.0 g, 95.1 mmol) is heated neat 10 minutes at 210° C. The mixture is cooled and acidified with 5N HCl, and then extracted with EtOAc, washed with brine, dried over sodium sulfate, and concentrated under reduced pressure to give the title compound (1.72 g, quantitative), which is utilized without purification. MS: (ES⁺) 224 (M+H); ¹H NMR (400 MHz, CDCl₃).

### Step B

### 6-Allyloxy-2-methyl-3-phenyl-benzofuran

A mixture of 2-methyl-3-phenyl-benzofuran-6-ol (1.59 g, 7.09 mmol), alkyl bromide (1.2 g, 9.93 mmol), and potassium carbonate (1.36 g, 9.93 mmol) in methyl ethyl ketone (50 mL) are heated at reflux overnight under nitrogen atmosphere. The mixture is concentrated under reduced pressure followed by the addition of water. The mixture is extracted with EtOAc, washed with brine, dried over sodium sulfate, and concentrated under reduced pressure. Purification by chromatography, eluting with EtOAc:hexane (4:1) provides the title compound (1.62 g, 86%). GC/MS: M.⁺ 264; ¹H NMR (400 MHz, CDCl₃).

### Step C

### 7-Allyl-2-methyl-3-phenyl-benzofuran-6-ol

The compound of Step B (1.62 g, 6.13 mmol) is dissolved in N,N-dimethylaniline and degassed with nitrogen, and then heated to reflux (192° C.) overnight. The mixture is cooled, diluted with EtOAc and washed with 1N HCl. Organic phase is washed with brine, dried over sodium sulfate, and concentrated under reduced pressure. Purification by recrystallization (toluene/hexane) provides the title compound (0.46 g, 28%). GC/MS: M.⁺ 264; ¹H NMR (400 MHz, CDCl₃).

### Step D

### 2-Methyl-3-phenyl-7-propyl-benzofuran-6-ol

The compound of Step C (260 mg, 0.98 mmol) is added in 2B ethanol (50 mL) to flask containing 10% Pd/C (90 mg) and the mixture is stirred about 2 hours under hydrogen filled balloon at rt. Catalyst is removed by filtration and the filtrate is concentrated under reduced pressure to give a mixture of title compound and over reduced material, 2-methyl-3-phenyl-7-propyl-2,3-dihydro-benzofuran-6-ol (200 mg). This mixture (200 mg) and 2,3 dichloro-5,6-dicyano 1,4benzoquinone (0.085 g, 0.37 mmol) is dissolved in dioxane (5 mL) and stirred overnight at rt. Water is added, and the mixture is extracted with EtOAc, washed with brine, dried over sodium sulfate, and concentrated under reduced pressure. Purification by chromatography, eluting with EtOAc:hexane (2:98) provides the title compound (0.040 g). MS: (ES⁻) 265 (M−H); ¹H NMR (400 MHz, CDCl₃).

### EXAMPLE 184

### Preparation of 4-Ethyl-2-phenylsulfanyl-phenol

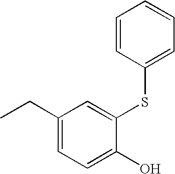

### Step A

### 4-Ethyl-2-thiophenyl anisole

4-Ethyl anisole (3.5 g, 25.7 mmol) and thiophenol (5.66 g, 51.4 mmol) are dissolved in 30 mL 1,1,1,3,3,3-hexafluoro-2-propanol. Bis(trifluoroacetoxy)iodo benzene (13.2 g 30.8 mmol) dissolved in 30 mL 1,1,1,3,3,3-hexafluoro-2-propanol is added dropwise to the solution while keeping the temperature near room temperature. The mixture is stirred for 30 minutes and then concentrated under reduced pressure. Purification by chromatography, eluting with EtOAc:hexane (3:97) provides the title compound (0.57 g, 15%). GC/MS: M.⁺ 244; ¹H NMR (400 MHz, CDCl₃).

### Step B

### 4-Ethyl-2-phenylsulfanyl-phenol

The compound of Step A (570 mg, 2.33 mmol) and tetrabutylammonium iodide (1.72 g, 4.67 mmol) in 25 mL is dissolved in DCM, and the mixture is cooled reaction to −78° C. Boron trichloride solution (4.7 ml, 1.0 M in DCM) is added dropwise over 5-10 minutes and stirred for 3 hours at 0° C. The mixture is quenched with ice water and stirred for 0.5 hours. The mixture is diluted with additional water and DCM. Organic layer is separated, washed with brine, dried over sodium sulfate, and concentrated under reduced pressure. Purification by chromatography, eluting with EtOAc:hexane (2.5:97.5) provides the title compound (0.41 g, 76%). GC/MS: M.⁺ 230; ¹H NMR (400 MHz, CDCl₃).

### EXAMPLE 185

### Procedure 1—General Procedures for Coupling and Hydrolysis

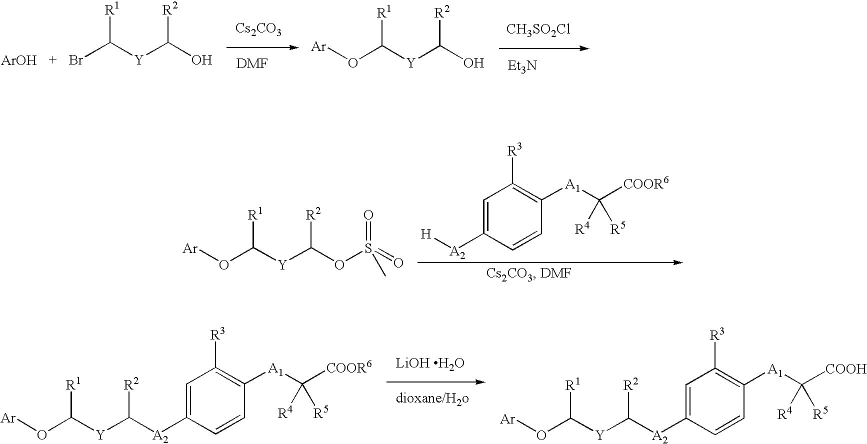

### EXAMPLE 186

### Procedure 2—General Procedures for Coupling and Hydrolysis

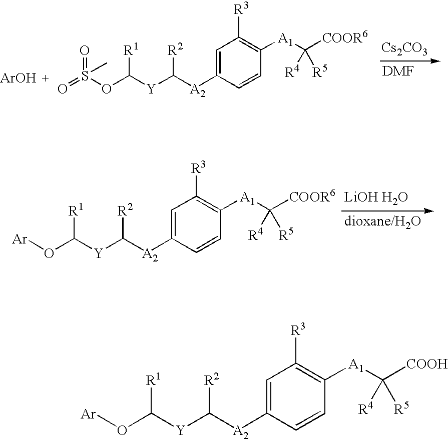

### EXAMPLE 187

### (R,S)-2-Methyl-2-(4-{3-[4-(4-trifluoromethyl-phenoxy)-phenoxy]-hexyloxy}-phenoxy)-propionic acid

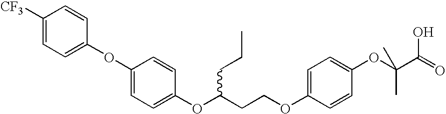

### Step A

### (R,S)-3-[4-(4-trifluoromethyl-phenoxy)-phenoxy]-hexan-1-ol

A mixture of 4-[4-(trifluoromethyl)phenoxy]phenol (1.4 g, 5.52 mmol), (R,S)-3-bromo-hexan-1-ol (1.0 g, 5.52 mmol), tetrabutyl ammonium iodide (1.0 g, 2.76 mmol), and cesium carbonate (3.6 g, 11.0 mmol) in 60 mL of DMF is heated overnight at 50° C. under nitrogen atmosphere. After cooling water is added, and the mixture is extracted with EtOAc, washed with brine, dried over sodium sulfate, and concentrated under reduced pressure. Purification by chromatography, eluting with EtOAc:hexane (1:4) provides the title compound (0.73 g, 36%). MS: (ES⁺) 709; ¹H NMR (400 MHz, CDCl₃).

### Step B

### (R,S)-2-methyl-2-(4-{3-[4-(4-trifluoromethyl-phenoxy)-phenoxy]-hexyloxy}-phenoxy)-propionic acid ethyl ester

The compound of 3-[4-(4-trifluoromethyl-phenoxy)-phenoxy]-hexan-1-ol (730 mg, 2.06 mmol) and TEA (0.34 mL, 2.47 mmol) are dissolved in 25 mL DCM, and the mixture is cooled to 0° C. followed by dropwise addition of MsCl (0.19 mL, 2.47 mmol). The mixture is stirred under nitrogen for 1.5 hours at 0° C. Water is added, and the organic layer is separated, washed with brine, dried over sodium sulfate, and concentrated under reduced pressure to give crude methanesulfonic acid 3-[4-(4-trifluoromethyl-phenoxy)-phenoxy]-hexyl ester (0.930 g) that is utilized without purification.

A mixture of methanesulfonic acid 3-[4-(4-trifluoromethyl-phenoxy)-phenoxy]-hexyl ester (144 mg, 0.33 mmol), 2-(4-hydroxy-phenoxy)-2-methyl-propionic acid ethyl ester (LLY 1433362) (74 mg 0.33 mmol), and cesium carbonate (280 mg, 0.66 mmol) in dry DMF (4 mL) is heated at 60° C. for 16 hours under nitrogen. The mixture is cooled and quenched with water. The mixture is extracted with EtOAc, washed with brine, dried over sodium sulfate, and concentrated under reduced pressure. Purification by flash chromatography, eluting with EtOAc:hexane (1:99), provides the title compound (0.11 g, 55%). MS: (ES⁺) 578; ¹H NMR (400 MHz, CDCl₃).

### Step C

### (R,S)-2-Methyl-2-(4-{3-[4-(4-trifluoromethyl-phenoxy)-phenoxy]-hexyloxy}-phenoxy)-propionic acid

Purified 2-methyl-2-(4-{3-[4-(4-trifluoromethyl-phenoxy)-phenoxy]-hexyloxy}-phenoxy)-propionic acid ethyl ester (110.0 mg, 0.196 mmol) (1 eq) is dissolved in 4 mL dioxane and lithium hydroxide hydrate (100.0 mg, 2.39 mmol) (12 eq) dissolved in 2 mL water is added. The mixture is stirred at rt overnight under nitrogen. The mixture is acidified with 5 N HCl, and water is added. The mixture is extracted into EtOAc, washed with brine, dried with sodium sulfate and concentrated under reduced pressure to give the title compound (0.101 g, 97%). Exact mass calcd for C₂₉H₂₅CF₃NO₆ (M+NH₄⁺): 550.2416, found 550.2426. ¹HNMR (400 MHz, CDCl₃).

### EXAMPLE 188

### (R,S)-2-{4-[3-(4-Ethyl-2-phenylsulfanyl-phenoxy)-butoxy]-phenoxy}-2-methyl-propionic acid

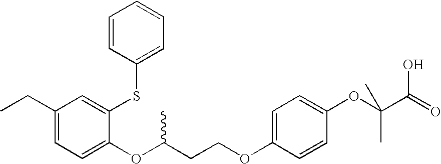

### Step A

### (R,S)-2-{4-[3-(4-ethyl-2-phenylsulfanyl-phenoxy)-butoxy]-phenoxy}-2-methyl-propionic acid ethyl ester

A mixture of 4-ethyl-2-phenylsulfanyl-phenol (Example 185) (98.4 mg, 0.43 mmol), (R,S)-2-[4-(3-methanesulfonyloxy-butoxy)-phenoxy]-2-methyl-propionic acid ethyl ester (160.0 mg 0.43 mmol), and cesium carbonate (347 mg, 1.07 mmol) in dry DMF (5 mL) is heated at 60° C. for 16 hours under nitrogen. The mixture is cooled and quenched with water. The mixture is extracted with EtOAc, washed with brine, dried over sodium sulfate, and concentrated under reduced pressure. Purification by flash chromatography, eluting with 7% EtOAc in hexane then 12% EtOAc in hexane (linear gradient), provides the title compound (0.067 g, 31%). MS:(ES⁺) 526 (M+NH₄⁺); ¹H NMR (400 MHz, CDCl₃).

### Step B

### (R,S)-2-{4-[3-(4-ethyl-2-phenylsulfanyl-phenoxy)-butoxy]-phenoxy}-2-methyl-propionic acid

Purified compound of Step A (67.0 mg, 0.13 mmol) (1 eq) is dissolved in 2 mL dioxane and lithium hydroxide hydrate (27.0 mg, 0.66 mmol) (˜5 eq) dissolved in 1 mL water is added. The mixture is stirred at rt overnight under nitrogen. The mixture is acidified with 5 N HCl, and water is added. The mixture is extracted into EtOAc, washed with brine, dried with sodium sulfate and concentrated under reduced pressure to give the title compound (24.0 mg, 74%). Mass (ES⁺): 481 (M+H⁺); ¹H NMR (400 MHz, CDCl₃).

### EXAMPLE 189

### 2-{4-[3-(R,S-2-Benzenesulfinyl-4-ethyl-phenoxy)-butoxy]-2-methyl-phenylsulfanyl}-2-methyl-propionic acid (enantiomer 1 and enantiomer 2)

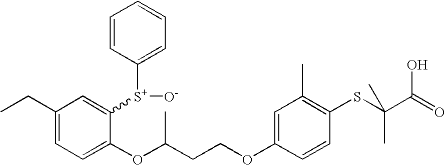

### Step A

### (R,S)-3-Bromo-butan-1-ol

A solution of ethyl beta-bromobutyrate (10.0 g, 51.3 mmol) in dry THF (100 mL) is cooled to −78° C. and treated dropwise with a 1M diisobutylaluminum hydride in toluene (107 mL, 107.7 mmol). The mixture is stirred for 15 minutes at −78° C. and then warmed to 0° C. and stirred for additional 45 minutes under nitrogen. The mixture is quenched slowly with 1 N HCl (200 mL) and then diluted with water, extracted with ether, washed with brine, dried over sodium sulfate, and concentrated under reduced pressure with bath at rt to give the title compound (6.1 g, 78%) that is utilized without purification. ¹H NMR (400 MHz, CDCl₃): δ=1.755 (d, 3H), δ=2.021 (m, 2H), δ=2.204 (s, 1H), δ=3.802 (t, 2H), δ=4.311 (m, 1H)

### Step B

### (R,S)-2-Benzenesulfinyl-4-ethyl-phenol

The compound of 4-ethyl-2-phenylsulfanyl-phenol (480 mg 2.08 mmol) is dissolved in 5 mL chloroform, and the mixture is cooled to 0° C. and solid meta-chloroperoxybenzoic acid (77%) (465 mg 2.08 mg) is added. The mixture is stirred about 10 minutes, and then quenched with water followed by the addition of ECM. The mixture is washed with saturated sodium bicarbonate and brine, dried over sodium sulfate, and concentrated under reduced pressure to give the title compound (0.51 g, quantitative). No purification is carried out. MS: (ES) 247 (M+H⁺); ¹H NMR (400 MHz, CDCl₃.

### Step C

### (R,S)-3-(2-Benzenesulfinyl-4-ethyl-phenoxy)-butan-1-ol

The title compound is prepared according to the procedure described in Example 187, Step A by using 3-bromo-butan-1-ol and 2-benzenesulfinyl-4-ethyl-phenol. Purification by flash chromatography, eluting with 50% EtOAc in hexane then to 70% EtOAc in hexane (linear gradient) provides the title compound (0.21 g, 30% yield). MS: (ES⁺) 319 (M+H⁺); ¹H NMR (400 MHz, CDCl₃).

### Step D

### (R,S)-Methanesulfonic acid 3-(2-benzenesulfinyl-4-ethyl-phenoxy)-butyl ester

The title compound (0.25 g, 95%) is prepared according to the procedure described in Example 187, Step B by using 3-(2-benzenesulfinyl-4-ethyl-phenoxy)-butan-1-ol. MS: (ES⁺) 397 (M+H⁺); ¹H NMR (400 MHz, CDCl₃).

### Step E

### (R,S)2-{4-[3-((R,S)2-Benzenesulfinyl-4-ethyl-phenoxy)-butoxy]-2-methyl-phenylsulfanyl}-2-methyl-propionic acid ethyl ester

The title compound is prepared according to the procedure described in Example 187, Step B by using methanesulfonic acid 3-(2-benzenesulfinyl-4-ethyl-phenoxy)-butyl ester and 2-(4-hydroxy-2-methyl-phenylsulfanyl)-2-methyl-propionic acid ethyl ester. Purification by flash chromatography, eluting with 20% EtOAc in hexane and then to 50% EtOAc in hexane (linear gradient) provides the title compound (0.055 g, 65%). MS:(ES⁺) 555 (M+H⁺); ¹H NMR (400 MHz, CDCl₃).

### Step F

### 2-{4-[3-((R,S)2-Benzenesulfinyl-4-ethyl-phenoxy)-butoxy]-2-methyl-phenylsulfanyl}-2-methyl-propionic acid (enantiomer pair 1 and enantiomer pair 2)

The compound obtained in Step F (55.0 mg, 0.099 mmol) (1 eq) is dissolved in 3 mL dioxane followed by the addition of lithium hydroxide hydrate (83.0 mg, 1.98 mmol) (˜20 eq) dissolved in 1.5 mL water. The mixture is stirred at rt overnight under nitrogen. The mixture is acidified with 5 N HCl and water is added, which then is extracted into EtOAc, washed with brine, dried with sodium sulfate and concentrated under reduced pressure. Purification by HPLC provides the title compounds (0.0107 g of enantiomer 1 and 0.0063 g of enamtiomer 2). Exact mass calcd for C₂₉H₃₅O₅S₂ (M+H⁺): 527.1926, found 527.1912. ¹HNMR (400 MHz, CDCl₃); Exact mass calcd for C₂₉H₃₅O₅S₂ (M+H⁺): 527.1926, found 527.1916. ¹H NMR (400 MHz, CDCl₃).

Examples 190 to 210 are prepared according to Procedure 1 (Example 185) or Procedure 2 (Example 186) and for the coupling and hydrolysis as exemplified in Examples 187-189.

### EXAMPLE 190

### (R,S)-3-{4-[3-(4′-Methoxy-biphenyl 4-yloxy)-hexyloxy]-2-methyl-phenyl}-propionic acid

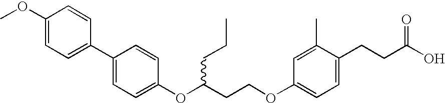

The title compound is prepared according to Procedure (Example 185). Exact mass calcd for C₂₉H₃₈NO₅ (M+NH₄⁺): 480.2750, found 480.2769; ¹HNMR (400 MHz, CDCl₃).

### EXAMPLE 191

### (R,S)-{4-[3-(4′-Methoxy-biphenyl-4-yloxy)-hexylsulfanyl]-2-methyl-phenoxy}-acetic acid

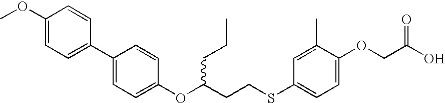

The title compound is prepared according to Procedure 1 (Example 185). MS(ES⁻): 479.15 (M−H); ¹HNMR (400 MHz, CDCl₃).

### EXAMPLE 192

### (R,S)-2-{4-[3-(4′-Methoxy-biphenyl-4-yloxy)-hexyloxy]-phenoxy}-2-methyl-propionic acid

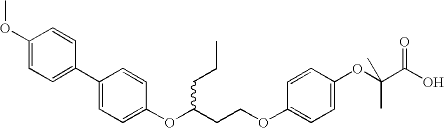

The title compound is prepared according to Procedure 1 (Example 185). Exact mass calcd for C₂₉H₃₈NO₆ (M+NH₄⁺): 496.2699, found 496.2697; ¹HNMR (400 MHz, CDCl₃).

### EXAMPLE 193

### (R,S)-2-{4-[3-(2-Cyclohexylmethyl-4-trifluoromethyl-phenoxy)-butoxy]-phenoxy}-2-methyl-propionic acid

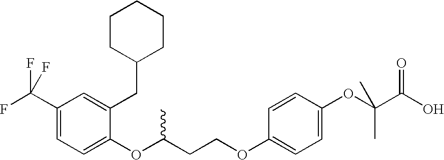

The title compound is prepared according to Procedure 2 (Example 186). Exact mass calcd for C₂₈H₃₉NO₅F₃ (M+NH₄⁺): 526.2780, found 526.2771; ¹HNMR (400 MHz, CDCl₃).

### EXAMPLE 194

### (R,S)-2-{4-[3-(2-Cyclopropylmethyl-4-trifluoromethyl-phenoxy)-butoxy]-phenoxy}-2-methyl-propionic acid

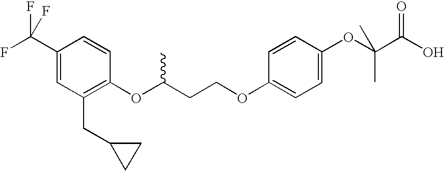

The title compound is prepared according to Procedure (Example 186). Exact mass calcd for C₂₅H₃₃NO₅F3 (M+NH₄⁺): 484.2311, found 484.2321; ¹HNMR (400 MHz, CDCl₃).

### EXAMPLE 195

### {6-[R,S-3-(R,S-2-Benzenesulfinyl-4-ethyl-phenoxy)-butoxy]-1-propyl-1H-indol-3-yl}-acetic acid

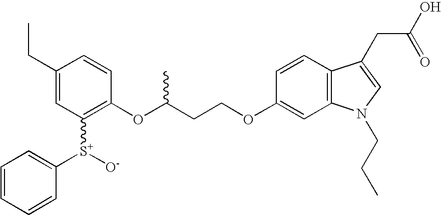

The title compound is prepared according to Procedure 1 (Example 185). MS (ES⁺): 534.4 (M+H⁺); ¹HNMR (400 MHz, CDCl₃).

### EXAMPLE 196

### (R,S)-2-{4-[3-(2,7-Dimethyl-3-phenyl-benzofuran-6-yloxy)-butoxy]-phenoxy}-2-methyl-propionic acid

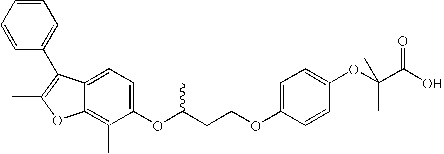

The title compound is prepared according to Procedure 2 (Example 186). Exact mass calcd for C₃₀H₃₃O₆ (M+H): 489.2277, found 489.2273; ¹H NMR (400 MHz, CDCl₃).

### EXAMPLE 197

### (R,S)-2-Methyl-2-{4-[3-(2-methyl-3-phenyl-7-propyl-benzofuran-6-yloxy)-butoxy]-phenoxy}-propionic acid

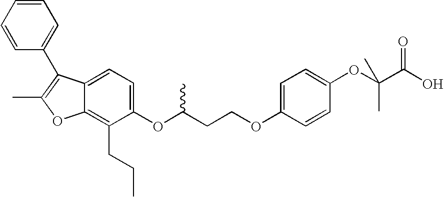

The title compound is prepared according to Procedure 2 (Example 186). Exact mass calcd for C₃₂H₃₇O₆ (M+H): 517.2590, found 517.2587; ¹H NMR (400 MHz, CDCl₃).

### EXAMPLE 198

### (R,S)-2-Methyl-2-{4-[3-(4-methyl-3-phenyl-7-propyl-benzofuran-6-yloxy)-butoxy]-phenoxy}-propionic acid

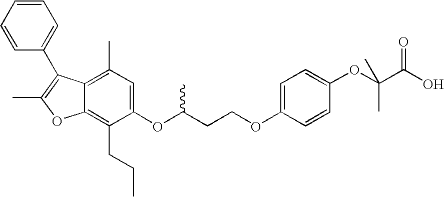

The title compound is prepared according to Procedure 2 (Example 186). Exact mass calcd for C₃₂H₃₇O₆ (M+H): 517.2590, found 517.2587; ¹H NMR (400 MHz, CDCl₃).

### EXAMPLE 199

### (R,S)-2-Methyl-2-{4-[3-(4-methyl-3-phenyl-benzofuran-6-yloxy)-butoxy]-phenoxy}-propionic acid

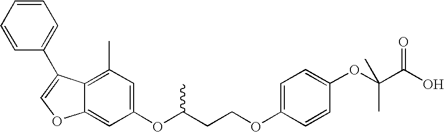

The title compound is prepared according to Procedure 2 (Example 186). Exact mass calcd for C₂₉H₃₁O₆ (M+H): 475.2121, found 475.2132; ¹H NMR (400 MHz, CDCl₃).

### EXAMPLE 200

### (R,S)-2-Methyl-2-(4-{3-[4-(4-trifluoromethyl-phenoxy)-phenoxy]-butoxy}-phenoxy)-propionic acid

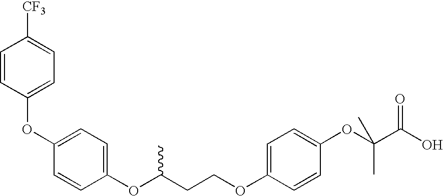

The title compound is prepared according to Procedure 2 (Example 186). Exact mass calcd for C₂₇H₃₁F₃NO₆ (M+NH₄⁺): 522.2103 found 522.2127; ¹H NMR (400 MHz, CDCl₃).

### EXAMPLE 201

### 2-Methyl-2-(4-{2-methyl-3-[4-(4-trifluoromethyl-phenoxy)-phenoxy]-propoxy}-phenoxy)-propionic acid

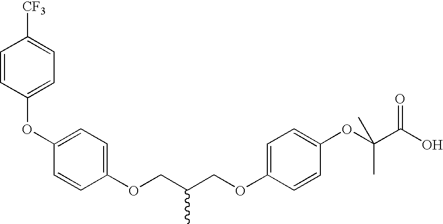

The title compound is prepared according to Procedure 2 (Example 186). Exact mass calcd for C₂₇H₃₁F₃NO₆ (M+NH₄⁺): 522.2103 found 522.2125; ¹H NMR (400 MHz, CDCl₃).

### EXAMPLE 202

### (R,S)-3-(2-Methyl-4-{3-[4-(4-trifluoromethyl-phenoxy)-phenoxy]-hexyloxy}-phenyl)-propionic acid

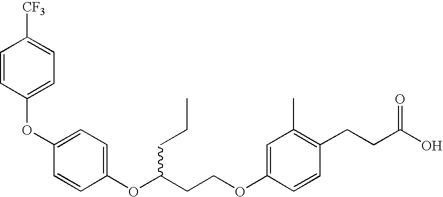

The title compound is prepared according to Procedure 1 (Example 185). MS (ES⁻): 515 (M−H); ¹H NMR (400 MHz, CDCl₃).

### EXAMPLE 203

### (R,S)-(2-Methyl-4-{3-[4-(4-trifluoromethyl-phenoxy)-phenoxy]-hexylsulfanyl}-phenoxy)-acetic acid

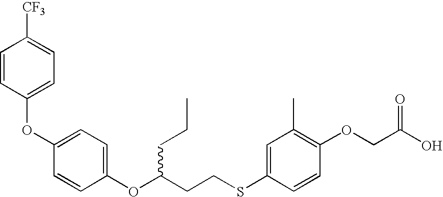

The title compound is prepared according to Procedure 1 (Example 185). MS (ES⁻): 533 (M−H); ¹H NMR (400 MHz, CDCl₃).

### EXAMPLE 204

### (R,S)-2-{4-[3-(2-Cyclopropylmethyl-4-trifluoromethyl-phenoxy)-butoxy]-2-methyl-phenoxy}-2-methyl-propionic acid

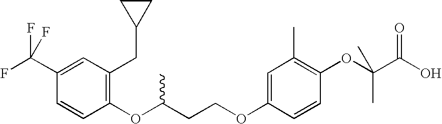

The title compound is prepared according to Procedure 1 (Example 185). Exact mass calcd for C₂₆H₃₅F₃NO₅ (M+NH₄⁺): 498.2467, found 498.2487; ¹H NMR (400 MHz, CDCl₃).

### EXAMPLE 205

### (R,S)-3-{4-[3-(2-Cyclopropylmethyl-4-trifluoromethyl-phenoxy)-butoxy]-2-methyl-phenyl}-propionic acid

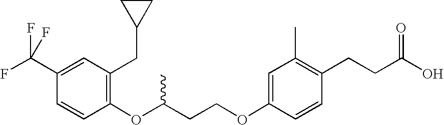

The title compound is prepared according to Procedure 1 (Example 185). Exact mass calcd for C₂₅H₃₃F₃NO₄ (M+NH₄⁺): 468.2362, found 468.2376; ¹H NMR (400 MHz, CDCl₃).

### EXAMPLE 206

### 3-{R-4-[3-(R,S-2-Benzenesulfinyl-4-ethyl-phenoxy)-butoxy]-2-methyl-phenyl}-propionic acid

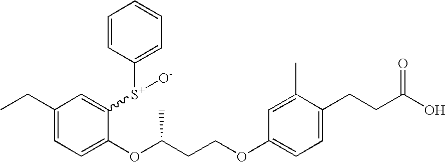

The title compound is prepared according to Procedure 2 (Example 186). Exact mass calcd for C₂₈H₃₃O₅S (M+H): 481.2049, found 481.2032; ¹H NMR (400 MHz, CDCl₃).

### EXAMPLE 207

### 3-{4-[3-(4-Ethyl-2-phenylsulfanyl-phenoxy)-butoxy]-2-methyl-phenyl}-propionic acid isomer 1

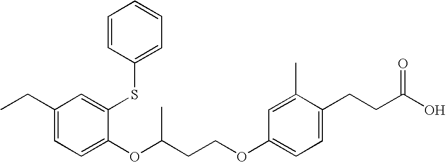

### Isomer 1

The title compound is prepared according to Procedure 2 (Example 186). Exact mass calcd for C₂₈H₃₆NO₄S (M+NH₄⁺): 482.2365, found 482.2358; ¹H NMR (400 MHz, CDCl₃).

### EXAMPLE 208

### 3-{4-[3-(4-Ethyl-2-phenylsulfanyl-phenoxy)-butoxy]-2-methyl-phenyl}-propionic acid isomer 2

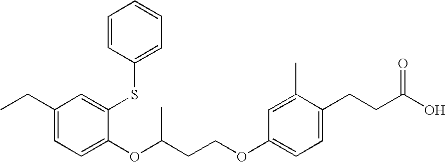

### Isomer 2

The title compound is prepared according to Procedure 2 (Example 186). Exact mass calcd for C₂₈H₃₆NO₄S (M+NH₄⁺): 482.2365, found 482.2375; ¹H NMR (400 MHz, CDCl₃).

### EXAMPLE 209

### (R,S)-3-{4-[3-(4-Ethyl-2-phenylsulfanyl-phenoxy)-butoxy]-2-methyl-phenyl}-propionic acid

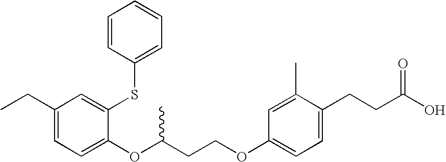

The title compound is prepared according to Procedure 2 (Example 186). Exact mass calcd for C₂₈H₃₆NO₄S (M+H⁺): 465.2117, found 465.2117; ¹H NMR (400 MHz, CDCl₃).

### EXAMPLE 210

### (R,S)-2-{4-[3-(4-Ethyl-2-phenylsulfanyl-phenoxy)-butoxy]-phenoxy}-2-methyl-propionic acid

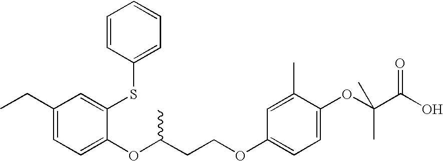

The title compound is prepared according to Procedure 2 (Example 186). Partial oxidation to the sulfoxide may occur under certain conditions. LC/MS: (linear gradient: 90% water/5% ACN/5% formic acid to 0% water/95% ACN/5% formic acid) single peak t_{R}=2.24 minutes, ES⁺ 495 (M+H); ¹H NMR (400 MHz, CDCl₃).

### EXAMPLE 211

### (R,S)-3-{4-[3-(R,S-2-Benzenesulfinyl-4-ethyl-phenoxy)-butoxy]-2-methyl-phenyl}-propionic acid

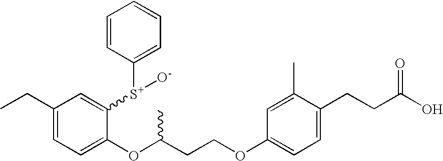

Pure (R,S)-3-{4-[3-(4-ethyl-2-phenylsulfanyl-phenoxy)-butoxy]-2-methyl-phenyl}-propionic acid (47.9 mg, 0.103 mmol, 1 equivalent) is dissolved in 5 mL chloroform, and the mixture is cooled to 0° C. and then solid 77% meta-chloroperoxybenzoic acid (22 mg, 0.098 mmol, 0.95 eq) is added. The mixture is stirred for about 10 minutes and quenched with water. DCM is added to the mixture. The mixture is washed with saturated sodium bicarbonate and brine, and then dried with sodium sulfate, and concentrated under reduced pressure to give the title compound (46.4 mg, 94%). Exact mass calcd for C₂₈H₃₃O₅S (M+H): 481.2049, found 481.2041; ¹H NMR (400 MHz, CDCl₃).

### EXAMPLE 212

### (R,S)-2-{4-[3-(R,S-2-Benzenesulfinyl-4-ethyl-phenoxy)-butoxy]-2-methyl-phenoxy}-2-methyl-propionic acid

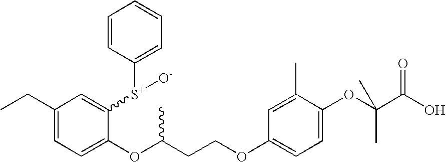

Pure (R,S)-2-{4-[3-(4-ethyl-2-phenylsulfanyl-phenoxy)-butoxy]-phenoxy}-2-methyl-propionic acid (Example 210) (54.4 mg, 0.110 mmol, 1 equivalent) is dissolved in 5 mL chloroform, and the mixture is cooled to 0° C., and solid 77% meta-chloroperoxybenzoic acid (23.4 mg, 0.104 mmol, 0.95 equivalent) is added. The mixture is stirred for about 10 minutes, quenched with water, and DCM is added. The mixture is washed with saturated sodium bicarbonate and brine, and then dried with sodium sulfate, and concentrated under reduced pressure to give the title compound (44.6 mg, 80%). Exact mass calcd for C₂₉H₃₅O₆S (M+H): 511.2154, found 511.2168; ¹H NMR (400 MHz, CDCl₃).

### EXAMPLE 213

### (R,S)-3-{4-[3-(2-Benzenesulfonyl-4-ethyl-phenoxy)-butoxy]-2-methyl-phenyl}-propionic acid

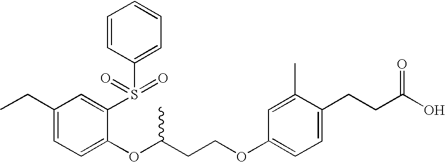

### Step A

### (R,S)-3-{4-[3-(2-Benzenesulfinyl-4-ethyl-phenoxy)-butoxy]-2-methyl-phenyl}-propionic acid methyl ester

The compound of (R,S)-3-{4-[3-(2-Benzenesulfonyl-4-ethyl-phenoxy)-butoxy]-2-methyl-phenyl}-propionic acid methyl ester (prepared by procedure 2) (60.0 mg, 0.125 mmol, 1 eq) is dissolved in 10 mL chloroform at rt and then solid 77% meta-chloroperoxybenzoic acid (70.0 mg, 0.312 mmol, 2.5 equivalents) is added. The mixture is stirred for an hour, quenched with water and DCM is added. The mixture is washed with 10% solution of sodium bisulfite, saturated sodium bicarbonate and brine, and then dried with sodium sulfate, and concentrated under reduced pressure. Purification by chromatography, eluting with 10% EtOAc in hexane to 20% EtOAc in hexane provides the title compound (41.7 mg, 65%). Ms: (ES⁺) 511 (N+H).

### Step B

### (R,S)-3-{4-[3-(2-Benzenesulfonyl-4-ethyl-phenoxy)-butoxy]-2-methyl-phenyl}-propionic acid

The title compound is prepared by using the compound obtained from Step A according to the procedure described in Example 187, Step C. Exact mass calcd for C₂₉H₃₂O₆SNa (M+Na) 519.1817, found 519.1830; ¹H NMR (400 MHz, CDCl₃).

### EXAMPLE 214

### 3-{4-[3-(2-Benzoyl-4-trifluoromethoxy-phenoxy)-butoxy]-2-methyl-phenyl}-propionic acid

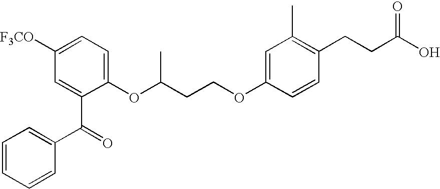

### Step A

### 3-{4-[3-(2-Benzoyl-4-trifluoromethoxy-phenoxy)-butoxy]-2-methyl-phenyl}-propionic acid methyl ester

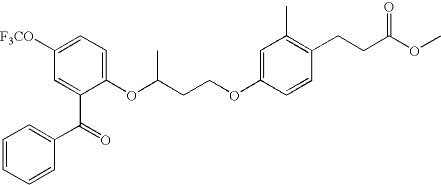

A mixture of (2-hydroxy-5-trifluoromethoxy-phenyl)-phenyl-methanone (0.94 g, 3.33 mmol), 3-[4-(3-methanesulfonyloxy-butoxy)-2-methyl-phenyl]-propionic acid methyl ester (1.38 g, 4.0 mmol) and Cs₂CO₃ (2.61 g, 8.0 mmol) in DMF (25 mL) is heated to 55° C. for 17 hr under N₂. The mixture is cooled to r.t. and diluted with Et₂O and filtered through a pad of celite. Organic layer is washed with 1N HCl, H₂O and brine, and then dried over Na₂SO₄, filtered and concentrated. Crude material is purified by chromatography (hexanes/acetone=10:1) to afford the title compound as colorless oil in 79% yield. R_{f}=0.4 (2/1 hexanes/acetone). ¹H NMR (400 MHz, CDCl₃).

### Step B

### 3-{4-[3-(2-Benzoyl-4-trifluoromethoxy-phenoxy)-butoxy]-2-methyl-phenyl}-propionic acid

A mixture of 3-{4-[3-(2-benzoyl-4-trifluoromethoxy-phenoxy)-butoxy]-2-methyl-phenyl}-propionic acid methyl ester (1.17 g, 2.19 mmol) and 4.4 mL of 5N NaOH (21.95 mmol) in 25 mL of EtOH is heated to reflux for 3 h. The mixture is cooled to r.t. and EtOH is removed under the vacuum. The residue is then dissolved in Et₂O and 1N HCl. Organic layer is washed with 1N HCl, H₂O and brine, and then dried over Na₂SO₄, filtered and concentrated. Crude material is submitted to chiral chromatography separation. Two enantiomers are separated using Chiralpak AD (4.6×250 mm) with 4:1 heptane/isopropanol with 0.1% TFA as the eluent (1 mL/min, UV280 nm). Isomer A: ¹H NMR (400 MHz, CDCl₃); HRMS (ES⁺) m/z exact mass calcd for C₂₈H₂₇F₃O₆ 516, found 517 (M+1, 100%). Isomer B: ¹H NMR (400 MHz, CDCl₃); HRMS (ES⁺) m/z exact mass calcd for C₂₈H₂₇F₃O₆ 516, found 517 (M+1, 100%).

### EXAMPLE 215

### 3-{4-[3-(2-Benzoyl-4-ethyl-phenoxy)-1-methyl-propoxy]-2-methyl-phenyl}-propionic acid

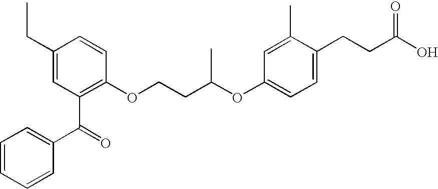

### Step A

### [5-Ethyl-2-(3-hydroxy-butoxy)-phenyl]-phenyl-methanone

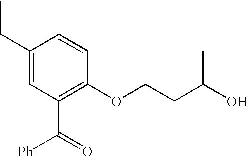

The mixture of (5-ethyl-2-hydroxy-phenyl)-phenyl-methanone (1.05 g, 4.6 mmol), toluene-4-sulfonic acid 3-hydroxy-butyl ester (1.25 g, 5.1 mmol) and Cs₂CO₃ (1.8 g, 5.6 mmol) in 25 mL of dry DMF is allowed to stand at 50° C. for overnight. The mixture is then cooled to r.t. and diluted with Et₂O and filtered through a pad of celite. Organic layer is washed with 1N HCl, H₂O and brine, and then dried over Na₂SO₄, filtered and concentrated. Crude material is purified by chromatography (hexanes/EtOAc=8:1) to afford the title compound as a colorless oil in 89% yield. R_{f}=0.29 (8/1 hexanes/EtOAc). ¹H NMR (400 MHz, CDCl₃).

### Step B

### Methanesulfonic acid 3-(2-benzoyl-4-ethyl-phenoxy)-1-methyl-propyl ester

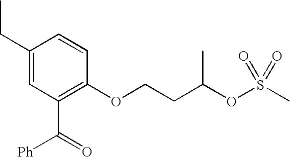

A mixture of [5-ethyl-2-(3-hydroxy-butoxy)-phenyl]-phenyl-methanone (0.85 g, 2.85 mmol), MsCl (0.33 mL, 4.27 mmol) and Et₃N (1.0 mL, 7.12 mmol) in 25 mL of dry CH₂Cl₂ is allowed to stand at 0° C. for 1 h and warmed up to r.t. for 2 h. The resulting mixture is washed with 1N HCl, H₂O and brine, and then dried over Na₂SO₄, filtered and concentrated. The crude material is used for next step without further purification. R_{f}=0.32 (8/1 hexanes/EtOAc). ¹H NMR (400 MHz, CDCl₃).

### Step C

### 3-{4-[3-(2-Benzoyl-4-ethyl-phenoxy)-1-methyl-propoxy]-2-methyl-phenyl}-propionic acid methyl ester

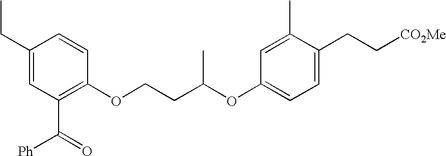

A mixture of methanesulfonic acid 3-(2-benzoyl-4-ethyl-phenoxy)-1-methyl-propyl ester (1.09 g, 2.90 mmol), 3-(4-hydroxy-2-methyl-phenyl)-propionic acid methyl ester (469 mg, 2.41 mmol) and Cs₂CO₃ (1.18 g, 3.62 mmol) in 25 mL of dry DMF is allowed to stand at 55° C. for overnight. The mixture is cooled to r.t. and diluted with Et₂O and filtered through a pad of celite. Organic layer is washed with 1N HCl, H₂O and brine, and then dried over Na₂SO₄, filtered and concentrated. Crude material is purified by chromatography (hexanes/acetone=10:1) to afford the title compound as a colorless oil in 62% yield. R_{f}=0.26 (10/1 hexanes/acetone). ¹H NMR (400 MHz, CDCl₃).

### Step D

### 3-{4-[3-(2-Benzoyl-4-ethyl-phenoxy)-1-methyl-propoxy]-2-methyl-phenyl}-propionic acid

A solution of 3-{4-[3-(2-benzoyl-4-ethyl-phenoxy)-1-methyl-propoxy]-2-methyl-phenyl}-propionic acid methyl ester (isomer 1 from chiral chromatography, 250 mg, 0.52 mmol) and 0.5 mL of 5N NaOH (2.63 mmol) in 10 mL of MeOH is allowed to stand at r.t. for 4 h. The organic solvent is removed under the vacuum. The residue is then dissolved in Et₂O and 1N HCl. Organic layer is washed with 1N HCl, H₂O, brine and dried over Na₂SO₄, filtered and concentrated to give the title compound in a colorless oil in 98% yield. ¹H NMR (400 MHz, CDCl₃); HRMS (ES⁺) m/z exact mass calcd for C₂₉H₃₂O₅ 460, found 461 (M+1, 100%).

### Step E

### 3-{4-[3-(2-Benzoyl-4-ethyl-phenoxy)-1-methyl-propoxy]-2-methyl-phenyl}-propionic acid

A solution of 3-{4-[3-(2-benzoyl-4-ethyl-phenoxy)-1-methyl-propoxy]-2-methyl-phenyl}-propionic acid methyl ester (isomer 2 from chiral chromatography, 241 mg, 0.50 mmol) and 0.5 mL of 5N NaOH (2.50 mmol) in 10 mL of MeOH is allowed to stand at r.t. for 4 h. The organic solvent is removed under the vacuum. The residue is then dissolved in Et₂O and 1N HCl. Organic layer is washed with 1N HCl, H₂O, brine, and then dried over Na₂SO₄, filtered and concentrated to give the title compound in a colorless oil in 98% yield. ¹H NMR (400 MHz, CDCl₃); HRMS (ES⁺) m/z exact mass calcd for C₂₉H₃₂O₅ 460, found 461 (M+1, 100%).

### EXAMPLE 215A

### 3-{2-Ethyl-4-[3-(4-ethyl-2-pyridin-2-yl-phenoxy)-butoxy]-phenyl}-propionic acid

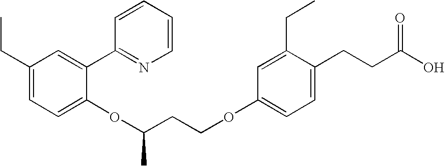

Cesium carbonate (0.091 g, 0.28 mmol) is added to 4-ethyl-2-pyridin-2-yl-phenol (0.04 g, 0.20 mmol) and 3-[2-ethyl-4-(3-methanesulfonyloxy-butoxy)-phenyl]propionic acid ethyl ester (0.09 g, 0.28 mmol) in DMF (5 mL), and the mixture is stirred under N₂ at 55° C. After 16 h, mixture is cooled to ambient temperature and filtered. The solid is washed with ethyl acetate. Filtrate is washed with water and saturated aqueous sodium chloride, and then dried over magnesium sulfate, filtered and concentrated under reduce pressure. Purification by flash chromatography, silica, hexanes:ethyl acetate (8:2) gives 3-{2-ethyl-4-[3-(4-ethyl-2-pyridin-2-yl-phenoxy)-butoxy]-phenyl}-propionic acid ethyl ester (0.045 g, 0.096 mmol, 48%): ES⁺ (m/e) 476.3 (M+H)⁺.

A 5 M aqueous solution of sodium hydroxide (0.30 mL, 1.50 mmol) is added to the above propionic acid ethyl ester (0.045 g, 0.10 mmol) in ethanol (3 mL), and the mixture is stirred at ambient temperature for 4 h. The mixture is acidified to pH=7 with a 1 M HCl and extracted with ethyl acetate. Organic layers were combined and washed with saturated aqueous sodium chloride, and then dried over magnesium sulfate, filtered and concentrated at reduced pressure to obtain title compound (0.035 g, 0.078 mmol, 82%): ES⁺ (m/e): 448.3 (M+H)⁺.

### EXAMPLE 216

### 3-{2-Methyl-4-[3-(2-pyridin-2-yl-4-trifluoromethyl-phenoxy)-butoxy]-phenyl}-propionic acid

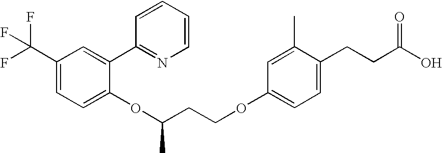

### Step A

### 2-methoxy-5-(trifluoromethyl)phenylboronic acid

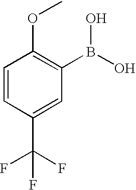

n-BuLi (1.6 M in hexane) (44.45 mL, 71.13 mmol) is added a solution of 2-bromo-1-methoxy-4-trifluoromethyl-benzene (18.14 g, 71.13 mmol) in diethylether (71 mL) at −78° C. and the mixture is stirred for an hour while maintaining the internal temperature below −75° C. The mixture is stirred at r.t. for 30 minutes, cooled to −78° C. and added over a solution of triisopropylborate (19.70 mL, 85.35 mmol) in diethylether (239 mL). The temperature is maintained below −75° C. for 1 h and stirred at r.t. for 30 minutes and concentrated HCl (200 mL) is added. The mixture is extracted with diethylether. Organic layers are combined, dried over sodium sulfate, filtered and concentrated under reduced pressure to give the title compound (quantitative).

### Step B

### 2-(2-Methoxy-5-trifluoromethyl-phenyl)-pyridine

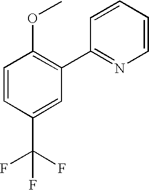

A mixture of 2-methoxy-5-(trifluoromethyl)-phenylboronic acid (15.64 g, 71.10 mmol), 2-bromopyridine (5.65 mL, 59.25 mmol), palladium tetrakis-(triphenylphosphine) (2.74 g, 2.37 mmol) and sodium carbonate (2 M in water) (83 mL, 165.9 mmol) in dimethoxyethane (118 mL) is stirred overnight under reflux. The mixture is cooled to rt and the layers are separated. The aqueous layer is extracted with ethylacetate and the organic layers are combined, dried, filtered and concentrated. Purification by flash chromatography, eluting with hexane: EtOAc 5:1 provides the title compound (11.68 g, 78%).

### Step C

### 2-Pyridin-2-yl-4-trifluoromethyl-phenol

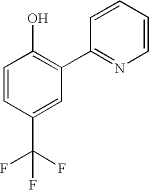

Boron tribromide (1.0 M in dichloromethane) (92.25 mL, 92.25 mmol) is added to a solution of 2-(2-methoxy-5-trifluoromethyl-phenyl)-pyridine (11.68 g, 46.12 mmol) in DCM (230 mL) at −78° C. The mixture is stirred at that temperature for 10 minutes, and the bath is removed and stirred at rt for 1 h. Water is added slowly and stirred for 1 h. The mixture is extracted with DCM and the organic layers are combined which is then dried over sodium sulfate, filtered and concentrated under reduced pressure. Purification by flash chromatography, eluting with hexane: EtOAc 5:1 provides the title compound (6.00 g, 54%).

### Step D

### 3-{2-Methyl-4-[3-(2-pyridin-2-yl-4-trifluoromethyl-phenoxy)-butoxy]-phenyl}-propionic acid

Cesium carbonate (0.091 g, 0.28 mmol) is added to 2-pyridin-2-yl-4-trifluoromethyl-phenol (0.045 g, 0.20 mmol) and 3-[4-(3-methanesulfonyloxy-butoxy)-2-methyl-phenyl]-propionic acid methyl ester (0.083 g, 0.24 mmol) in DMF (3 mL), and the mixture is stirred under N₂ at 55° C. After 16 h, the mixture is cooled to ambient temperature, filtered and washed solid with EtOAc. The filtrate is washed with water and saturated aqueous sodium chloride, and then dried over magnesium sulfate, filtered and concentrated under reduce pressure. Purification by flash chromatography, silica, hexanes: EtOAc (8:2) provides 3-{2-methyl-4-[3-(2-pyridin-2-yl-4-trifluoromethyl-phenoxy)-butoxy]-phenyl}-propionic acid methyl ester (0.045 g, 0.092 mmol, 46%): ES⁺ (m/e) 488.2 (M+H)⁺.

Aqueous solution of sodium hydroxide (5M, 0.25 mL, 1.2 mmol) is added to the above propionic acid methyl ester (0.041 g, 0.08 mmol) in methanol (3 mL), and the mixture is stirred at ambient temperature for 4 h. The mixture is acidified to pH=7 with a 1 M HCl and extracted with EtOAc. Organic layers are combined and washed with saturated aqueous sodium chloride dry over magnesium sulfate, filtrated and concentrated at reduced pressure to obtain title compound (0.040 g, 0.085 mmol, 100%): ES⁺ (m/e):474.2 (M+H)⁺.

### EXAMPLE 217

### 3-{2-Methyl-4-[3-(2-pyridin-4-yl-4-trifluoromethyl-phenoxy)-butoxy]-phenyl}-propionic acid

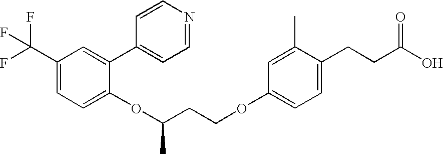

### Step A

### 4-(2-Methoxy-5-trifluoromethyl-phenyl)-pyridine

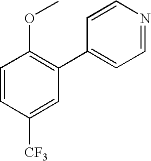

Na₂CO₃ 2M in H₂O (2.8 mmol) and Pd(PPh₃)₄ (4%) are added to a solution of the bromide (1 mmol) and boronic acid (1.4 mmol) in DME (2 mL/mmol). The mixture is stirred at 85° C. overnight. The crude is quenched with H₂O and extracted with AcOEt. The combined organic layers are dried over Na₂SO₄, filtered, evaporated and purified by column chromatography using the appropriate eluent.

Bromide: 4-Bromo-pyridine hydrochloride (1.76 g, 9.09 mmol). Boronic acid: 2-methoxy-5-(trifluoromethyl)-phenylboronic acid (2.00 g, 9.09 mmol) Eluent: Hexane:AcOEt 1:1.

### Step B

### 2-Pyridin-4-yl-4-trifluoromethyl-phenol

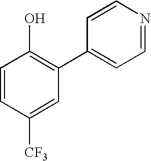

To a solution of the methoxy derivative (1 mmol) in CH₂Cl₂ (5 mL/mmol) at −78° C. under N₂, BBr₃ 1.0M (CH₂Cl₂) is added (2 mmol). After 10 min, the bath is removed, and the mixture is stirred at rt. After 1-2 h, water is added. The crude is extracted with CH₂Cl₂. The organic layer is dried over Na₂SO₄, filtered, evaporated and purified by flash chromatography using the eluent. Methoxyderivative: 4-(2-Methoxy-5-trifluoromethyl-phenyl)-pyridine (0.77 g, 3.04 mmol). Eluent: Hexane:AcOEt 1:2. ¹H NMR (CDCl₃, 300 MHz): δ 7.10 (d, 1H, J=8.48 Hz), 7.51 (dd, 1H, J=2.02, 8.48 Hz), 7.62 (s, 1H), 7.74 (d, 2H, J=6.05 Hz), 8.62 (d, 1H, J=6.05 Hz). MS [M+H] 239.9.

### Step C

### 3-{2-Methyl-4-[3-(2-pyridin-4-yl-4-trifluoromethyl-phenoxy)-butoxy]-phenyl}-propionic acid

The title compound is prepared according to the procedure described in Example 216 by using 3-{2-methyl-4-[3-(2-pyridin-4-yl-4-trifluoromethyl-phenoxy)-butoxy]-phenyl}-propionic acid methyl ester. MS:ES+(m/e) 474.2 (M⁺).

### EXAMPLE 218

### 3-{2-Ethyl-4-[3-(2-pyridin-2-yl-trifluoromethyl-phenoxy)-butoxy]-phenyl}-propionic acid

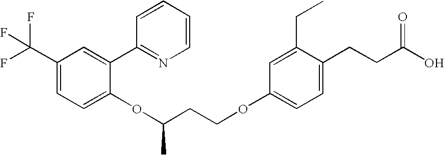

The title compound is prepared according to the procedure described in Example 215A by using 3-{2-ethyl-4-[3-(2-pyridin-2-yl-4-trifluoromethyl-phenoxy)-butoxy]-phenyl}-propionic acid ethyl ester. MS:ES+(m/e):488.2 (M⁺).

### EXAMPLE 219

### 3-{2-Ethyl-4-[3-(2-pyridin-4-yl-4-trifluoromethyl-phenoxy)-butoxy]-phenyl}-propionic acid

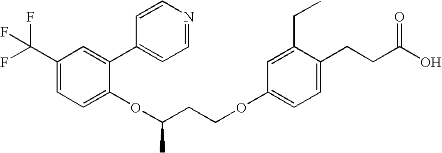

The title compound is prepared according to the procedure described in Example 215A by using 3-{2-ethyl-4-[3-(2-pyridin-4-yl-4-trifluoromethyl-phenoxy)-butoxy]-phenyl}-propionic acid ethyl ester. MS:ES+(m/e):488.2 (M⁺).

### EXAMPLE 220

### 3-{4-[3-(2-Benzo[d]isoxazol-3-yl-4-chloro-phenoxy)-butoxy]-2-methyl-phenyl}-propionic acid

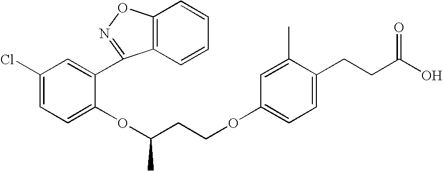

### Step A

### (5-Chloro-2-methoxy-phenyl)-(2-fluoro-phenyl)-methanone

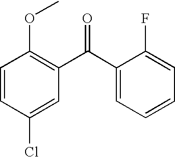

To a solution of the anisole (1 mmol) in CH₂Cl₂ (1 mL/mmol), under N₂ at 0° C., AlCl₃ (1.2 mmol) is d in several portions. After stirring for 10 min, acyl chloride (1.1 mmol) is added. The mixture is stirred for 2-3 h and is poured into an ice:water:HCl mixture. The organic layer is washed with saturated NaHCO₃ and water, dried over Na₂SO₄, filtered, evaporated and purified by flash chromatography using the eluent. Anisole: 1-Chloro-4-methoxy-benzene (2.00 g, 14.03 mmol). Acylchloride: 2-Fluoro-benzoyl chloride (2.45 g, 15.45 mmol). Eluent: Hexane:AcOEt 10:1.

### Step B

(5-Chloro-2-methoxy-phenyl)-(2-fluoro-phenyl)-methanone oxime

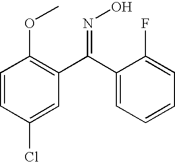

A solution of (5-chloro-2-methoxy-phenyl)-(2-fluoro-phenyl)-methanone (1.73 g, 6.52 mmol) in warm ethanol (26.0 mL) is treated with NH₂OH.HCl (2.18 g, 31.32 mmol) and refluxed for 6 h. The mixture is poured into water and cooled in an ice:water bath. The oxime is filtered off and dried in vacuo.

### Step C

### 3-(5-Chloro-2-methoxy-phenyl)-benzo[d]isoxazole

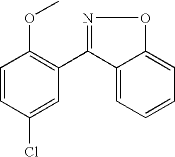

To a solution of (5-chloro-2-methoxy-phenyl)-(2-fluoro-phenyl)-methanone oxime (1.64 g, 5.86 mmol) in 1-methyl-pyrrolidin-2-one (26.0 mL), potassium t-butoxide (0.72 g, 6.45 mmol) is added. The mixture is heated at 100° C. for 3 h. The crude is diluted with water and extracted with AcOEt. The organic layer is washed with water and brine, and then dried over Na₂SO₄, filtered, evaporated, purified by flash chromatography using hexane:AcOEt 5:1 as eluent.

### Step D

### 2-Benzo[d]isoxazol-3-yl-4-chloro-phenol

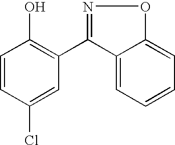

To a solution of the methoxy derivative (1 mmol) in CH₂Cl₂ (5 mL/mmol) at −78° C. under N₂, BBr₃ 1.0M (CH₂Cl₂) is added (2 mmol). After 10 min the bath is removed and the mixture is stirred at rt. After 1-2 h, water is added and the crude is extracted with CH₂Cl₂. The organic layer is dried over Na₂SO₄, filtered and evaporated. The crude product is purified by flash chromatography using the eluent. Methoxyderivative: 3-(5-Chloro-2-methoxy-phenyl)-benzo[d]isoxazole (0.57 g, 2.20 mmol). Eluent: Hexane:AcOEt 5:1. ¹H NMR (CDCl₃, 300 MHz): 7.05 (d, 1H, J=8.88 Hz), 7.30 (dd, 1H, J=2.42, 8.88 Hz), 7.38-7.43 (m, 1H), 7.62 (m, 2H), 7.85 (d, 1H, J=2.43 Hz), 7.99 (d, 1H, J=8.28 Hz). MS [M+H] 246.1.

### Step E

### 3-{4-[3-(2-Benzo[d]isoxazol-3-yl-4-chloro-phenoxy)-butoxy]-2-methyl-phenyl}-propionic acid

The title compound is prepared according to the procedure described in Example 216 by using 3-{4-[3-(2-benzo[d]isoxazol-3-yl-4-chloro-phenoxy)-butoxy]-2-methyl-phenyl}-propionic acid methyl ester gives the title compound. MS:ES+(m/e) 480.2 (M⁺).

### EXAMPLE 221

### 3-{4-[3-(4-Chloro-2-pyridin-4-yl-phenoxy)-butoxy]-2-methyl-phenyl}-propionic acid

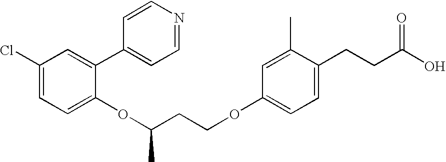

### Step A

### 3-Chloro-6-methoxy-benzene boronic acid

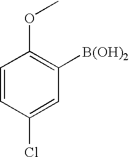

To a solution of the bromide (1 mmol) in THF (1 mL/mmol) at −78° C. under N₂, n-BuLi (1.2 mmol) is added and then B(OPrⁱ)₃ (2 mmol) is added after 15-30 min. The mixture is stirred for 3 h at rt. The crude is quenched with HCl. The aqueous layer is extracted with AcOEt, and the organic layers are combined, dried over Na₂SO₄, filtered and evaporated. The boronic acids are used without further purification in the next step. Bromide: 2-Bromo-4-chloro-1-methoxy-benzene (0.50 g, 2.26 mmol); nBuLi (1.6 M, Hex): 1.69 mL, 2.71 mmol; Triisopropylborate: 1.04 mL, 4.52 mmol. ¹H NMR (300 MHz, CDCl₃): 3.80 (s, 3H), 6.73 (d, 1H, J=8.9 Hz), 7.16 (d, 1H, J=8.9 Hz), 7.70 (s, 1H). R_{f}=0.4 (hex:AcOEt 5:1).

### Step B

### 4-(5-Chloro-2-methoxy-phenyl)-pyridine

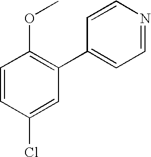

To a solution of the bromide (1 mmol) and boronic acid (1.4 mmol) in DME (2 mL/mmol), Na₂CO₃ 2M in H₂O (2.8 mmol) and Pd(PPh₃)₄ (4%) are added. The mixture is stirred at 85° C. overnight. The crude is quenched with H₂O and extracted with AcOEt. The combined organic layers are dried over Na₂SO₄, filtered and evaporated. The crude product is purified by column chromatography using the appropriate eluent. Bromide: 4-bromo-pyridine hydrochloride (3.50 g, 17.88 mmol); Boronic acid: 3-Chloro-6-methoxy-benzene boronic acid (4.00 g, 21.46 mmol); Eluent: Hexane:AcOEt 1:1.

### Step C

### 4-Chloro-2-pyridin-4-yl-phenol

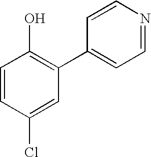

To a solution of the methoxy derivative (1 mmol) in CH₂Cl₂ (5 mL/mmol) at −78° C. under N₂, BBr₃ 1.0M (CH₂Cl₂) is added (2 mmol). After 10 min., the bath is removed and the mixture is stirred at rt. After 1 to 2 h, water is added, and the crude is extracted with CH₂Cl₂. The organic layer is dried over Na₂SO₄, filtered and evaporated. The crude product is purified by flash chromatography using the eluent indicated in each case. Methoxyderivative: 4-(5-Chloro-2-methoxy-phenyl)-pyridine (1.10 g, 5.00 mmol); Eluent: AcOEt. ¹H NMR (CDCl₃, 300 MHz): 6.99 (d, 1H, J=8.67 Hz), 7.21 (d, 1H, J=8.67 Hz); 7.33 (s, 1H), 7.71 (d, 2H, J=5.05 Hz), 8.60 (d, 1H, J=4.64 Hz). MS [M+H] 206.1.

### Step D

### 3-{4-[3-(4-Chloro-2-pyridin-4-yl-phenoxy)-butoxy]-2-methyl-phenyl}-propionic acid

The title compound is prepared according to the procedure described in Example 216 by using 3-{4-[3-(4-chloro-2-pyridin-4-yl-phenoxy)-butoxy]-2-methyl-phenyl}-propionic acid methyl ester. MS:ES+(m/e) 440.1 (M⁺).

### EXAMPLE 222

### {4-[3-(2-Benzo[d]isoxazol-3-yl-4-chloro-phenoxy)-butoxy]-2-methyl-phenylsulfanyl}-acetic acid

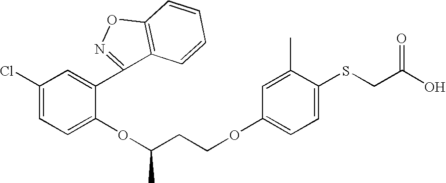

The title compound is prepared according to the procedure described in Example 215A by using {4-[3-(2-benzo[d]isoxazol-3-yl-4-chloro-phenoxy)-butoxy]-2-methyl-phenylsulfanyl}-acetic acid ethyl ester. MS:ES+(m/e):498.0 (M⁺).

### EXAMPLE 223

### 3-{2-Ethyl-4-[3-(2-pyridin-3-yl-4-trifluoromethyl-phenoxy)-butoxy]-phenyl}-propionic acid

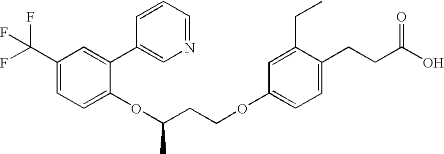

### Step A

### 2-Pyridin-3-yl-4-trifluoromethyl-phenol

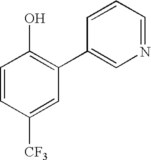

To a solution of the bromide (1 mmol) and boronic acid (1.4 mmol) in DME (2 mL/mmol), Na₂CO₃ 2M in H₂O (2.8 mmol) and Pd(PPh₃)₄ (4%) are added The mixture is stirred at 85° C. overnight. The crude is quenched with H₂O and extracted with AcOEt. The combined organic layers are dried over Na₂SO₄, filtered and evaporated. The crude product is purified by column chromatography using the appropriate eluent in each case. Bromide: 2-Bromo-4-trifluoromethyl-phenol (4.70 g, 19.52 mmol); Boronic acid: 3-pyridine boronic acid (2.40 g, 19.52 mmol); Eluent: Hexane:AcOEt 1:2. ¹H NMR (DMSO, 300 MHz): 6.96 (d, 1H, J=8.5 Hz), 7.34 (dd, 1H, J=4.8, 7.7 Hz), 7.60 (d, 1H, J=8.1 Hz), 7.76 (dd, 1H, J=2.4, 8.5 Hz), 7.85 (m, 1H), 7.98 (d, 1H, J=2.4 Hz), 8.45 (dd, 1H, J=1.6, 6.4 Hz), 8.64 (d, 1H, J=2.0 Hz). MS [4+H] 239.8.

### Step B

### 3-{2-Ethyl-4-[3-(2-pyridin-3-yl-4-trifluoromethyl-phenoxy)-butoxy]-phenyl}-propionic acid

The title compound is prepared according to the procedure described in Example 215A by using 3-{2-ethyl-4-[3-(2-pyridin-3-yl-4-trifluoromethyl-phenoxy)-butoxy]-phenyl}-propionic acid ethyl ester. MS:ES+(m/e):488.2 (M⁺).

### EXAMPLE 224

### 2-Methyl-2-{4-[3-(7-phenyl-naphthalen-2-yloxy)-butoxy]-phenoxy}-propionic acid

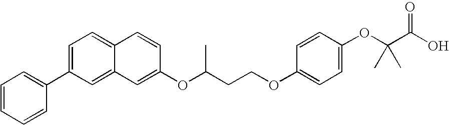

### Step A

### 4-Methyl-[1,3,2]dioxathiane 2-oxide

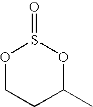

Thionyl chloride (15.9 mL, 217 mmol) is added drop wise over 1 h to a 0° C. solution of 1,3-butanediol (15 mL, 167 mmol) in methylene chloride (80 mL) and vented to a sodium hydroxide scrubber. The resulting solution, while still vented to the scrubber, is refluxed for 1 h and cooled to ambient temperature. The solution is washed thoroughly with water, saturated aqueous sodium bicarbonate, and more water. The organic layer is dried over MgSO₄ and concentrated in vacuo over a cool water bath to provide 17.5 g (77%) of the title compound.

### Step B

### 4-Methyl-[1,3,2]dioxathiane 2,2-dioxide

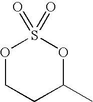

Ruthenium (III) chloride (0.365 g, 1.76 mmol) is added to a biphasic solution of 4-methyl-[1,3,2]dioxathiane 2-oxide) (10.9 g, 80.1 mmol) and sodium periodate (34.3 g, 160.1 mmol) in carbon tetrachloride (150 mL), water (230 mL) and ACN (150 mL). The reaction suspension is stirred at ambient temperature for 2 h, and then extracted from the aqueous layer with methylene chloride. The organic layer is filtered through a pad of celite, dried over MgSO₄, and concentrated in vacuo to provide 12.0 g (99%) of the title compound. ¹H NMR (400 MHz, CDCl₃) δ 5.04-4.98 (m, 1H), 4.73 (tt, 1H, J=10.9 Hz, 2.7 Hz), 4.56-4.52 (m, 1H), 2.15-2.03 (m, 1H), 1.87 (dt, 1H, J=14.0 Hz, 1.9 Hz), 1.44 (dd, 3H, J=6.3 Hz, 2.8 Hz).

### Step C

### 2-[4-(3-Hydroxy-butoxy)-phenoxy]-2-methyl-propionic acid ethyl ester

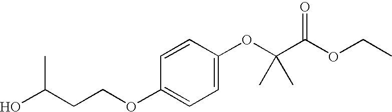

A 0° C. solution of 4-methyl-[1,3,2]dioxathiane 2,2-dioxide (8.1 g, 53.2 mmol) in ACN (300 mL) is treated with cesium carbonate (29.5 g, 79.8 mmol) and 2-(4-hydroxy-phenoxy)-2-methyl-propionic acid ethyl ester (29.5 g, 90.5 mmol). The mixture is stirred at ambient temperature for 10 h and concentrated in vacuo. The reaction residue, which is partitioned between diethyl ether and concentrated HCl, is stirred rapidly for 10 h at ambient temperature. The organic layer is washed with water, saturated aqueous sodium bicarbonate and brine, and then dried over Na₂SO₄ and concentrated in vacuo. The crude material is purified by flash chromatography to provide 10.2 g (65%) of the title compound.

### Step D

### 2-[4-(3-Methanesulfonyloxy-butoxy)-phenoxy]-2-methyl-propionic acid ethyl ester

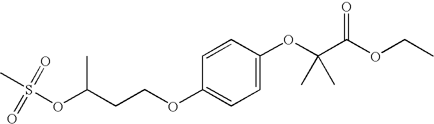

Methanesulphonyl chloride (3.2 mL, 41.3 mmol) is added to a 0° C. solution of 2-[4-(3-hydroxy-butoxy)-phenoxy]-2-methyl-propionic acid ethyl ester (10.2 g, 34.4 mmol) and TEA (7.2 mL, 51.6 mmol) in methylene chloride (300 mL). The resulting solution is stirred at 0° C. for 2 h, and then quenched with 1N HCl. The organic layer is washed with 1N HCl, dried over Na₂SO₄, and concentrated in vacuo. The crude material is purified by flash chromatography, using 25% acetone in hexanes as eluent, to provide 11.12 g (86%) of the title compound. ¹H NMR (400 MHz, CDCl₃) δ 7.15 (d, 2H, J=9.1 Hz), 6.85 (d, 2H, J=9.1 Hz), 4.22 (q, 2H, J=7.1 Hz), 3.09 (s, 3H), 2.92 (s, 1H), 1.58 (s, 6H), 1.52 (s, 1H), 1.50 (d, 1H, J=6.4 Hz), 1.26 (t, 2H, J=7.1 Hz), 1.24 (t, 2H, J=7.4 Hz). MS [EI+] 392 (M+NH₄)⁺. R_{f}=0.18 in 33% acetone in hexanes.

### Step E

### 2-Methoxy-7-phenyl-naphthalene

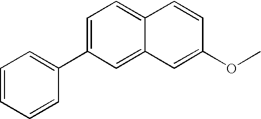

A flame-dried reaction vessel is charged with trifluoro-methanesulfonic acid 7-methoxy-naphthalen-2-yl ester (1.10 g, 3.59 mmol), phenyl boronic acid (1.31 g, 10.8 mmol), tricyclohexyphosphine (0.151 g, 0.54 mmol), palladium (II) acetate (0.081 g, 0.36 mmol), and cesium fluoride (4.91 g, 32.3 mmol). ACN (35 mL) is added to the reaction vessel, and the reaction suspension is heated at 90° C. for 6 minutes, and then cooled to ambient temperature and filtered through celite. The filtrate is diluted with methylene chloride, washed with saturated aqueous sodium bicarbonate, dried over MgSO₄, and concentrated in vacuo. The crude material is purified by flash chromatography, using 5% acetone in hexanes as eluent, to provide 0.65 g (77%) of the title compound. ¹H NMR (400 MHz, CDCl₃) δ 7.97 (d, 1H, J=1.7 Hz), 7.85 (d, 1H, J=9.2 Hz), 7.78 (d, 1H, J=8.8 Hz), 7.74 (dd, 2H, J=8.4 Hz, 1.3 Hz), 7.62 (dd, 1H, J=8.4 Hz, 1.7 Hz), 7.50 (t, 2H, J=7.9 Hz), 7.41 (t, 1H, J=7.9 Hz), 7.22 (d, 1H, 3=2.5 Hz), 7.17 (dd, 1H, J=9.2 Hz, 2.5 Hz). MS [EI+] 235 (M+H)⁺. R_{f}=0.52 in 33% acetone in hexanes.

### Step F

### 7-Phenyl-naphthalen-2-ol

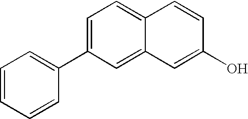

A mixture of 2-methoxy-7-phenyl-naphthalene (0.65 g, 2.77 mmol) and pyridine HCl (6.41 g, 55.5 mmol) is melted at 205° C. for 45 minutes. The reaction mixture is cooled to ambient temperature, diluted with CH₂Cl₂, and washed with 1N HCl. The organic layer is dried over Na₂SO₄, and concentrated in vacuo. The crude material is purified by flash chromatography, using 17% acetone in hexanes as eluent, to provide 0.61 g (100%) of the title compound. ¹H NMR (400 MHz, CDCl₃) δ 7.88 (s, 1H), 7.84 (d, 1H, J=8.2 Hz), 7.78 (d, 1H, J=8.7 Hz), 7.71 (d, 2H, J=8.2 Hz), 7.60 (dd, 1H, J=8.2 Hz, 1.4 Hz), 7.48 (t, 2H, J=8.2 Hz), 7.38 (t, 1H, J=8.2 Hz), 7.20 (d, 1H, J=2.4 Hz), 7.10 (dd, 1H, J=8.7 Hz, 2.4 Hz). MS [EI−] 219 (M−H)⁺. R_{f}=0.30 in 33% acetone in hexanes.

### Step G

### 2-Methyl-2-{4-[3-(7-phenyl-naphthalen-2-yloxy)-butoxy]-phenoxy}-propionic acid

A solution of 7-phenyl-naphthalen-2-ol (Step F) (0.033 g, 0.15 mmol) and 2-[4-(3-methanesulfonyloxy-butoxy)-phenoxy]-2-methyl-propionic acid ethyl ester (Step 1) (0.056 g, 0.15 mmol) in DMF (3 mL) is treated with cesium carbonate (0.58 g, 0.18 mmol) and heated to 50° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography using 17% acetone in hexanes as eluent, to provide a quantitative yield of the ester. R_{f}=0.38 in 33% acetone in hexanes.

A solution of 2-methyl-2-{4-[3-(7-phenyl-naphthalen-2-yloxy)-butoxy]-phenoxy}-propionic acid ethyl ester and 5N NaOH (0.5 mL) in ethanol (5 mL) is refluxed under N₂ for 30 minutes, and then cooled to ambient temperature and concentrated in vacuo. The reaction residue is dissolved in DCM, washed with 1N HCl, dried over Na₂SO₄, and concentrated in vacuo to provide the title compound. ¹H NMR (400 MHz, CDCl₃) δ7.85 (d, 1H, J=1.2 Hz), 7.81 (d, 1H, J=9.4 Hz), 7.74 (d, 1H, J=9.4 Hz), 7.70 (dd, 2H, J=8.8 Hz, 1.2 Hz), 7.59 (dd, 1H, J=8.8 Hz, 1.8 Hz), 7.48 (t, 2H, J=8.2 Hz), 7.37 (t, 1H, J=8.2 Hz), 7.24 (d, 1H, J=1.8 Hz), 7.13 (dd, 1H, J=8.8 Hz, 2.4 Hz), 6.89 (d, 2H, J=9.4 Hz), 6.82 (d, 2H, J=9.4 Hz), 4.87-4.82 (m, 1H), 4.18-4.09 (m, 2H), 2.29-2.22 (m, 1H), 2.18-2.11 (m, 1H), 1.50 (s, 6H), 1.46 (d, 3H, J=6.1 Hz). HRMS (ES⁺) m/z exact mass calcd for C30H31O5 471.2171, found 471.2187.

### EXAMPLE 225

### 2-Methyl-2-{4-[2-methyl-3-(7-phenyl-naphthalen-2-yloxy)-propoxy]-phenoxy}-propionic acid

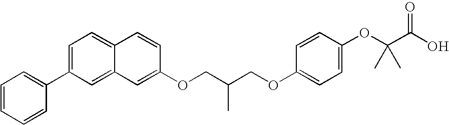

### Step A

### Methanesulfonic acid 3-methanesulfonyloxy-2-methyl-propyl ester

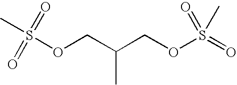

Methanesulphonyl chloride (5.2 mL, 66.6 mmol) is added to a 0° C. solution of 2-methyl-1,3-propanediol (5 mL, 55.5 mmol) and TEA (11.6 mL, 83.2 mmol) in methylene chloride (200 mL). The resulting solution is stirred at 0° C. for 2 h and quenched with 1N HCl. The organic layer is washed with 1N HCl, dried over Na₂SO₄, and concentrated in vacuo to provide 8.8 g (65%) of the title compound. ¹H NMR (400 MHz, CDCl₃) δ 4.19 (d, 1H, J=4.8 Hz), 4.17 (d, 1H, J=4.8 Hz), 4.12 (d, 1H, J=6.3 Hz), 4.10 (d, 1H, J=6.3 Hz), 2.99 (s, 6H), 2.34-2.27 (m, 1H), 1.04 (d, 3H, J=6.3 Hz). MS [EI+] 264 (M+NH₄)⁺. R_{f}=0.08 in 33% acetone in hexanes.

### Step B

### 2-[4-(3-Methanesulfonyloxy-2-methyl-propoxy)-phenoxy]-2-methyl-propionic acid ethyl ester

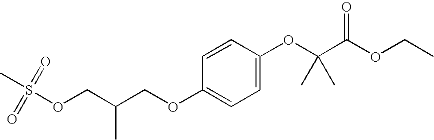

A solution of methanesulfonic acid 3-methanesulfonyloxy-2-methyl-propyl ester (8.8 g, 35.7 mmol) and 2-(4-hydroxy-phenoxy)-2-methyl-propionic acid ethyl ester (1.09 g, 35.73 mmol) in DMF (20 mL) is treated with cesium carbonate (3.5 g, 10.7 mmol) and heated to 50° C. under N₂. After 10 h, the mixture is cooled to ambient temperature and diluted with EtOAc. The organic layer is washed with 1N HCl, water and brine, and then dried over MgSO₄, and concentrated in vacuo. The crude material is purified by flash chromatography, using 20% acetone in hexanes as eluent to provide 1.62 g (60%) of the title compound. ¹H NMR (400 MHz, CDCl₃) δ 7.81 (d, 2H, J=9.2 Hz), 7.74 (d, 2H, J=9.2 Hz), 4.27, 4.26 (AB_{q}, 2H, J=5.3 Hz, 3.8 Hz), 4.88 (d, 1H, J=5.0 Hz, isomer 1), 4.85 (d, 1H, J=5.0 Hz, isomer 2), 4.82 (d, 1H, J=6.1 Hz, isomer 1), 4.79 (d, 1H, J=6.1 Hz, isomer 2), 2.95 (s, 3H), 2.38-2.31 (m, 1H), 1.51 (s, 6H), 1.26 (t, 3H, J=7.3 Hz0, 1.10 d, 3H, J=6.9 Hz). MS [EI+] 392 (M+NH₄)⁺. R_{f}=0.12 in 33% acetone in hexanes.

### Step C

### 2-Methyl-2-{4-[2-methyl-3-(7-phenyl-naphthalen-2-yloxy)-propoxy]-phenoxy}-propionic acid

A solution of 7-phenyl-naphthalen-2-ol (0.036 g, 0.16 mmol) and 2-[4-(3-methanesulfonyloxy-2-methyl-propoxy)-phenoxy]-2-methyl-propionic acid ethyl ester (0.065 g, 0.16 mmol) in DMF (3 mL) is treated with cesium carbonate (0.62 g, 0.20 mmol) and heated to 50° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography using 17% acetone in hexanes as eluent to provide the ester. R_{f}=0.35 in 33% acetone in hexanes.

A solution of 2-methyl-2-{4-[2-methyl-3-(7-phenyl-naphthalen-2-yloxy)-propoxy]-phenoxy}-propionic acid and 5N NaOH (0.5 mL) in ethanol (5 mL) is refluxed under N₂ for 30 minutes, and then cooled to ambient temperature and concentrated in vacuo. The reaction residue is dissolved in DCM, washed with 1N HCl, dried over Na₂SO₄, and concentrated in vacuo to provide the title compound. ¹H NMR (400 MHz, CDCl₃) δ 7.85 (d, 1H, J=1.7 Hz), 7.81 (d, 1H, J=8.9 Hz), 7.74 (d, 1H, J=8.9 Hz), 7.70 (d, 2H, J=7.3 Hz0, 7.60 (dd, 1H, J=8.9 Hz, 2.2 Hz), 7.48 (t, 2H, J=7.8 Hz), 7.38 (d, 1H, J=7.8 Hz), 7.24 (d, 1H, J=2.2 Hz), 7.13 (dd, 1H, J=8.9 Hz, 2.2 Hz), 6.89 (d, 2H, J=9.5 Hz), 6.82 (d, 2H, J=9.5 Hz), 4.84 (q, 1H, J=6.1 Hz), 4.18-4.09 (m, 2H), 2.30-2.22 (m, 1H), 2.18-2.11 (m, 1H), 1.50 (s, 6H), 1.46 (d, 3H, J=6.1 Hz). HRMS (ES⁺) m/z exact mass calcd for C30H31NO5 471.2171, found 471.2187.

### EXAMPLE 226

### 2-{4-[3-(2-Benzoyl-4-trifluoromethoxy-phenoxy)-butoxy]-phenoxy}-2-methyl-propionic acid

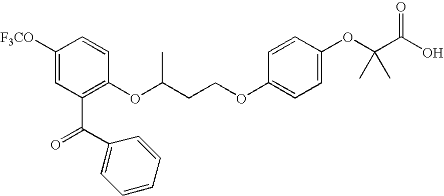

### Step A

### (2-Methoxy-5-trifluoromethoxy-phenyl)-phenyl-methanone

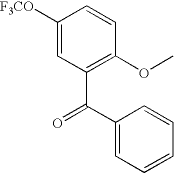

n-Butyl lithium (32.5 mL, 52 mmol) is added drop wise to −1° C. TMEDA (7.85 mL, 52 mmol) in a flame-dried reaction vessel over 30 minutes. The compound of 4-(trifluoromethoxy)anisole (3.94 mL, 26 mmol) is added drop wise to the resulting yellow mixture. This resulting brown solution is stirred at −10° C. for thirty minutes and treated with N-methoxy-N-methylbenzamide (7.92 mL, 52 mmol). The mixture is stirred at 10° C. for 40 minutes, and then quenched with 1N HCl and diluted with diethyl ether. The organic layer is washed with 1N HCl, water and brine, and then dried over Na₂SO₄, and concentrated in vacuo. The crude material is purified by flash chromatography using 11% acetone in hexanes as eluent to provide 5.38 g (70%) of the title compound. ¹H NMR (400 MHz, CDCl₃) δ 7.80 (dd, 2H, J=8.4 Hz, 1.3 Hz), 7.58 (tt, 1H, J=8.0 Hz, 1.3 Hz), 7.45 (t, 2H, J=8.0 Hz), 7.33 (dd, 1H, J=8.4 Hz, 3.0 Hz), 7.24 (d, 1H, J=3.0 Hz), 6.99 (d, 1H, J=9.3 Hz), 3.73 (s, 3H). R_{f}=0.42 in 33% acetone in hexanes.

### Step B

### (2-Hydroxy-5-trifluoromethoxy-phenyl)-phenyl-methanone

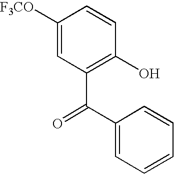

A mixture of (2-methoxy-5-trifluoromethoxy-phenyl)-phenyl-methanone (5.38 g, 18.6 mmol) and pyridine HCl (42 g, 36.3 mmol) is melted at 205° C. for 3.5 h. The mixture is cooled to ambient temperature, diluted with CH₂Cl₂, and washed with 1N HCl. The organic layer is dried over Na₂SO₄ and concentrated in vacuo. The crude material is purified by flash chromatography using 17% acetone in hexanes as eluent to provide 2.71 g (71%) of the title compound. ¹H NMR (400 MHz, CDCl₃) δ 11.93 (s, 1H), 7.69 (dd, 2H, J=8.4 Hz, 1.5 Hz), 7.64 (tt, 1H, J=7.5 Hz, 2.1 Hz), 7.55 (d, 2H, J=8.1 Hz), 7.47 (d, 1H, J=2.7 Hz), 7.40 (dd, 1H, J=8.7 Hz, 2.7 Hz), 7.10 (d, 1H, J=9.3 Hz). MS [EI−] 281 (M−H)⁺. R_{f}=0.56 in 33% acetone in hexanes.

### Step C

### 2-{4-[3-(2-Benzoyl-4-trifluoromethoxy-phenoxy)-butoxy]-phenoxy}-2-methyl-propionic acid

A solution of (2-hydroxy-5-trifluoromethoxy-phenyl)-phenyl-methanone (0.044 g, 0.16 mmol) and 2-[4-(3-methanesulfonyloxy-butoxy)-phenoxy]-2-methyl-propionic acid ethyl ester (0.058 g, 0.16 mmol) in DMF (2.9 mL) is treated with cesium carbonate (0.066 g, 0.20 mmol) and heated to 50° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography using 17% acetone in hexanes as eluent to provide the ester. R_{f}=0.33 in 33% acetone in hexanes.

A solution of 2-{4-[3-(2-benzoyl-4-trifluoromethoxy-phenoxy)-butoxy]-phenoxy}-2-methyl-propionic acid ethyl ester and 5N NaOH (0.3 mL) in ethanol (3 mL) is refluxed under N₂ for 30 minutes, and then cooled to ambient temperature and concentrated in vacuo. The reaction residue is dissolved in DCM, washed with 1N HCl, dried over Na₂SO₄, and concentrated in vacuo to provide 0.021 g of the title compound. ¹H NMR (400 MHz, CDCl₃) δ 7.78, 7.74 (AB_{q}, 2H, J=7.6 Hz), 7.53 (t, 1H, J=7.6 Hz), 7.40 (t, 2H, J=7.6 Hz), 7.26 (d, 2H, J=1.9 Hz), 6.98 (d, 1H, J=8.9 Hz), 6.87 (d, 2H, J=8.9 Hz), 6.66 (d, 2H, J=8.9 Hz), 4.62 (q, 1H, J=6.4 Hz), 3.73-3.68 (m, 2H), 1.86-1.82 (m, 2H), 1.54 (s, 6H), 1.20 (d, 3H, J=6.4 Hz). MS [EI+] 533 (M+H)⁺.

### EXAMPLE 227

### 2-{4-[3-(2-Benzoyl-4-trifluoromethoxy-phenoxy)-2-methyl-propoxy]-phenoxy}-2-methyl-propionic acid

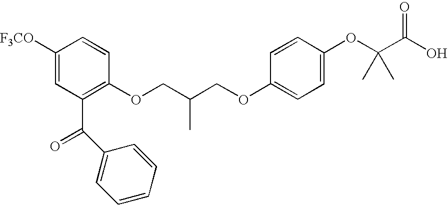

A solution of (2-hydroxy-5-trifluoromethoxy-phenyl)-phenyl-methanone (0.061 g, 0.22 mmol) and 2-[4-(3-methanesulfonyloxy-2-methyl-propoxy)-phenoxy]-2-methyl-propionic acid ethyl ester (0.081 g, 0.22 mmol) in DMF (2.9 mL) is treated with cesium carbonate (0.092 g, 0.28 mmol) and heated to 50° C. under N₂. After 10 h, the mixture is cooled to ambient temperature and diluted with diethyl ether. The organic layer is washed with 1N HCl, water and brine, and then dried over Na₂SO₄ and concentrated in vacuo. The crude material is purified by flash chromatography using 17% acetone in hexanes as eluent to provide the ester. R_{f}=0.33 in 33% acetone in hexanes.

A solution of 2-{4-[3-(2-benzoyl-4-trifluoromethoxy-phenoxy)-2-methyl-propoxy]-phenoxy}-2-methyl-propionic acid ethyl ester and 5N NaOH (0.3 mL) in ethanol (3 mL) is refluxed under N₂ for 30 minutes, then cooled to ambient temperature and concentrated in vacuo. The reaction residue is dissolved in DCM, washed with 1N HCl, dried over Na₂SO₄, and concentrated in vacuo to provide 0.054 g of the title compound. ¹H NMR (400 MHz, CDCl₃) δ 7.77, 7.73 (AB_{q}, 2H, J=7.3 Hz), 7.49 (t, 1H, J=7.3 Hz), 7.36 (t, 2H, J=7.3 Hz), 7.27 (d, 2H, J=3.0 Hz), 6.97 (d, 1H, J=9.1 Hz), 6.87 (d, 2H, J=9.1 Hz), 6.62 (d, 2H, J=9.1 Hz), 3.93 (t, 2H, J=5.5 Hz), 3.45 (q, 2H, J=2.4 Hz), 2.14-2.06 (m, 1H), 1.55 (s, 6H), 0.80 (d, 3H, J=6.7 Hz). MS [EI+] 533 (M+H)⁺.

### EXAMPLE 228

### 2-{4-[3-(3-Benzoyl-naphthalen-2-yloxy)-butoxy]-phenoxy}-2-methyl-propionic acid

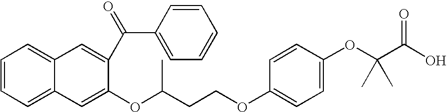

### Step A

### (3-Hydroxy-naphthalen-2-yl)-phenyl-methanone

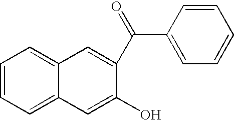

Phenyllithium (95 mL, 1.8M in 70/30 cyclohexane/ether) is added drop wise to a −78° C. solution of 3-hydroxy-2-naphthoic acid (4.0 g, 21.3 mmol) in THF. The mixture is warmed to ambient temperature for 3 h, and then cooled to 0° C. and quenched with water. Then resulting bright yellow solution is stirred vigorously while 1 N HCl is added. The organic layer is washed with water and brine, and then dried over MgSO₄ and adsorbed onto silica gel. The crude material is purified by flash chromatography, using 20% acetone in hexanes as eluent, to provide the title compound. ¹H NMR (400 MHz, CDCl₃) δ 11.15 (s, 1H), 8.17 (s, 1H), 7.78 (d, 2H, J=7.4 Hz), 7.73 (d, 1H J=5.0 Hz), 7.71 (d, 1H, J=5.0 Hz), 7.66 (tt, 1H, J=8.1 Hz), 7.55-7.52 (m, 3H), 7.39 (s, 1H), 7.32 (t, 2H, J=8.1 Hz). HRMS (ES⁺) m/z exact mass calcd for C28H29NO6S2Cl 574.1125, found 574.1122. R_{f}=0.44 in 33% acetone in hexanes.

### Step B

### 2-{4-[3-(3-Benzoyl-naphthalen-2-yloxy)-butoxy]-phenoxy}-2-methyl-propionic acid

A solution of (3-hydroxy-naphthalen-2-yl)-phenyl-methanone (0.042 g, 0.17 mmol) and 2-[4-(3-methanesulfonyloxy-butoxy)-phenoxy]-2-methyl-propionic acid ethyl ester (0.064 g, 0.17 mmol) in DMF (3 mL) is treated with cesium carbonate (0.072 g, 0.22 mmol) and heated to 50° C. under N₂. After 10 h, the mixture is cooled to ambient temperature and diluted with EtOAc. The organic layer is washed with 1N HCl and water, dried through a Varian ChemElut cartridge, and concentrated in vacuo. The crude material is purified by flash chromatography, using 20% acetone in hexanes as eluent, to provide the ester. R_{f}=0.26 in 33% acetone in hexanes.

A solution of 2-{4-[3-(3-benzoyl-naphthalen-2-yloxy)-butoxy]-phenoxy}-2-methyl-propionic acid ethyl ester and 5N NaOH (0.3 mL) in ethanol (3.5 mL) is refluxed under N₂, then cooled to ambient temperature and concentrated in vacuo. The reaction residue is dissolved in DCM, washed with 1N HCl, dried through a Varian ChemElut cartridge, and concentrated in vacuo to provide 0.034 g of the title compound. ¹H NMR (400 MHz, CDCl₃) δ 7.89 (s, 1H), 7.82-7.75 (m, 4H), 7.53-7.46 (m, 2H), 7.39 (td, 1H, J=8.2 Hz, 1.5 Hz), 7.34 (t, 2H, J=8.2 Hz), 7.23 (s, 1H), 6.87 (d, 2H, J=8.9 Hz), 6.63 (d, 2H, J=8.9 Hz), 3.78 (q, 1H, J=5.7 Hz), 3.73 (td, 2H, J=5.7 Hz, 1.9 Hz), 2.17 (s, 1H), 1.89 (q, 2H, J=6.2 Hz), 1.51 (s, 6H), 1.27 (d, 3H, J=6.2 Hz). MS [EI+] 499 (M+H)⁺.

### EXAMPLE 229

### 2-{4-[3-(3-Benzoyl-naphthalen-2-yloxy)-2-methyl-propoxy]-phenoxy}-2-methyl-propionic acid

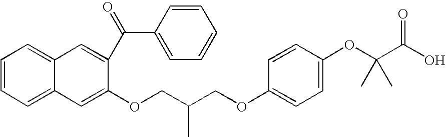

A solution of (3-hydroxy-naphthalen-2-yl)-phenyl-methanone (0.042 g, 0.17 mmol) and 2-[4-(3-methanesulfonyloxy-2-methyl-propoxy)-phenoxy]-2-methyl-propionic acid ethyl ester (0.063 g, 0.17 mmol) in DMF (3 mL) is treated with cesium carbonate (0.071 g, 0.22 mmol) and heated to 50° C. under N₂. After 10 h, the mixture is cooled to ambient temperature and diluted with EtOAc. The organic layer is washed with 1N HCl and water, dried through a Varian ChemElut cartridge, and concentrated in vacuo. The crude material is purified by flash chromatography, using 20% acetone in hexanes as eluent, to provide the ester. R_{f}=0.24 in 33% acetone in hexanes.

A solution of 2-{4-[3-(3-benzoyl-naphthalen-2-yloxy)-2-methyl-propoxy]-phenoxy}-2-methyl-propionic acid ethyl ester and 5N NaOH (0.3 mL) in ethanol (3.5 mL) is refluxed under N₂, then cooled to ambient temperature and concentrated in vacuo. The residue is dissolved in DCM, washed with 1N HCl, dried through a Varian ChemElut cartridge, and concentrated in vacuo. The material is lost while purifying by flash chromatography. ¹H NMR (400 MHz, CDCl₃) δ 7.89 (s, 1H), 7.79 (q, 4H, J=9.1 Hz), 7.53-7.46 (m, 2H), 7.39 (td, 1H, J=7.0 Hz, 0.7 Hz), 7.34 (t, 2H, J=7.7 Hz), 7.22 (s, 1H), 6.87 (d, 2H, J=9.1 Hz), 6.64 (d, 2H, J=9.1 Hz), 4.04 (p, 2H, J=5.9 Hz), 3.50 (d, 2H, J=6.8 Hz), 2.18 (p, 2H, J=5.9 Hz), 1.53 (s, 6H), 0.85 (d, 3H, J=6.8 Hz). MS[EI+] 499 (M+H)⁺.

### EXAMPLE 230

### 3-{4-[3-(2-Benzoyl-4-trifluoromethoxy-phenoxy)-butoxy]-2-methyl-phenyl}-propionic acid

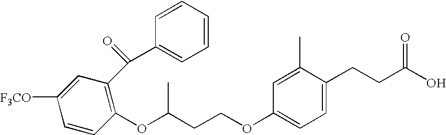

### Step A

### Toluene-4-sulfonic acid 3-hydroxy-butyl ester

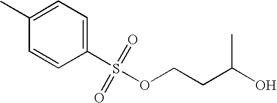

A solution of 1,3-butanediol (2 mL, 22.3 mmol), TEA (3.1 mL, 22.3 mmol), and p-toluene sulphonyl chloride (4.25 g, 22.3 mmol) in methylene chloride (45 mL) is treated with dibutyltin oxide (0.111 g, 0.45 mmol) and stirred under N₂ for 10 h. The reaction suspension is filtered through a pad of celite and concentrated in vacuo. The crude material is purified by flash chromatography, using 20% acetone in hexanes as eluent, to provide 3.44 g (63%) of the title compound. MS [EI+] 245 (M+H)⁺. R_{f}=0.18 in 33% acetone in hexanes.

### Step B

### 3-[4-(3-Hydroxy-butoxy)-phenyl]-propionic acid methyl ester

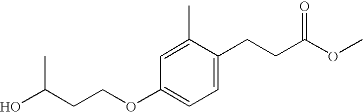

A solution of (toluene-4-sulfonic acid 3-hydroxy-butyl ester (3.5 g, 14.3 mmol) and (2.3 g, 11.9 mmol) in DMF (40 mL) is treated with cesium carbonate (5.8 g, 17.8 mmol) and heated to 5° C. under N₂. After 10 h, the mixture is cooled to ambient temperature and diluted with diethyl ether. The organic layer is washed with 1N HCl and water, dried over Na₂SO₄, and concentrated in vacuo. The crude material is purified by flash chromatography, using 17% acetone in hexanes as eluent, to provide 1.51 g (48%) of the title compound. ¹H NMR (400 MHz, CDCl₃) δ 7.03 (d, 1H, J=8.2 Hz), 6.71 (d, 1H, J=2.5 Hz), 6.67 (dd, 1H, J=8.2 Hz, 2.5 Hz), 4.15-4.02 (m, 3H), 3.67 (s, 3H), 2.87 (t, 2H, J=7.5 Hz), 2.54 (t, 2H, J=7.5 Hz), 2.35 (d, 1H, J=4.1 Hz), 2.28 (s, 3H), 1.89 (q, 2H, J=6.3 Hz), 1.25 (d, 3H, J=6.3 Hz). R_{f}=0.31 in 33% acetone in hexanes.

### Step C

### 3-[4-(3-Methanesulfonyloxy-butoxy)-2-methyl-phenyl]-propionic acid methyl ester

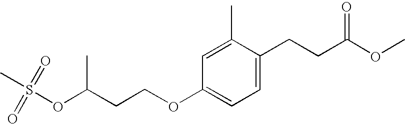

Methanesulphonyl chloride (0.53 mL, 6.8 mmol) is added to a 0° C. solution of 3-[4-(3-Hydroxy-butoxy)-phenyl]-propionic acid methyl ester (1.51 g, 5.67 mmol) and TEA (1.2 mL, 8.5 mmol) in methylene chloride (60 mL). The resulting solution is stirred at 0° C. for 10 h and quenched with 1N HCl. The organic layer is washed with 1N HCl, dried over Na₂SO₄, and concentrated in vacuo to provide 1.9 g (100%) of the title compound. ¹H NMR (400 MHz, CDCl₃) δ 7.03 (d, 1H, J=8.3 Hz), 6.69 (d, 1H, J=2.4 Hz), 6.65 (dd, 1H, J=8.3 Hz, 2.4 Hz), 5.04 (q, 1H, J=6.3 Hz), 4.03 (t, 2H, J=5.0 Hz), 3.67 (s, 3H), 2.93 (s, 3H), 2.87 (t, 2H, J=7.5 Hz), 2.54 (t, 2H, J=7.5 Hz), 2.28 (s, 3H), 2.10 (qd, 2H, J=6.3 Hz, 2.3 Hz), 1.51 (d, 3H, J=6.3 Hz). MS [EI+] 362 (+H)⁺. R_{f}=0.18 in 33% acetone in hexanes.

### Step D

### 3-{4-[3-(2-Benzoyl-4-trifluoromethoxy-phenoxy)-butoxy]-2-methyl-phenyl}-propionic acid

A solution of (2-hydroxy-5-trifluoromethoxy-phenyl)-phenyl-methanone (0.14 g, 0.47 mmol) and 3-[4-(3-methanesulfonyloxy-butoxy)-2-methyl-phenyl]-propionic acid methyl ester(0.24 g, 0.71 mmol) in DMF (5 mL) is treated with cesium carbonate (0.262 g, 0.80 mmol) and heated to 50° C. under N₂. After 10 h, the reaction mixture is cooled to ambient temperature and diluted with EtOAc. The organic layer is washed with 1N HCl, water and brine, and then dried over Na₂SO₄, and concentrated in vacuo. The crude material is purified by flash chromatography, using 17% acetone in hexanes as eluent, to provide the ester. R_{f}=0.28 in 33% acetone in hexanes.

A solution of 3-{4-[3-(2-Benzoyl-4-trifluoromethoxy-phenoxy)-butoxy]-2-methyl-phenyl}-propionic acid methyl ester and 5N NaOH (0.4 mL) in ethanol (4 mL) is refluxed under N₂, then cooled to ambient temperature and concentrated in vacuo. The reaction residue is dissolved in DCM, washed with 1N HCl, dried over Na₂SO₄, and concentrated in vacuo to provide 0.115 g of the title compound. ¹H NMR (400 MHz, CDCl₃) δ 7.74 (d, 2H, J=7.0 Hz), 7.52 (t, 1H, J=7.0 Hz), 7.38 (t, 2H, J=7.8 Hz), 7.25 (d, 2H, J=7.8 Hz), 6.96 (d, 2H, J=8.7 Hz), 6.55 (d, 1H, J=2.6 Hz), 6.49 (dd, 1H, J=8.7 Hz, 2.6 Hz), 4.59 (q, 1H, J=6.0 Hz), 3.67 (t, 2H, J=6.0 Hz), 2.81 (t, 2H, J=8.3 Hz), 2.51 (t, 2H, J=8.3 Hz), 2.22 (s, 3H), 1.79 (q, 2H, J=6.0 Hz), 1.16 (d, 3H, J=6.0 Hz). MS [EI+] 517 (M+H)⁺. R_{f}=0.54 in 10% methanol in methylene chloride.

### EXAMPLE 231

### 3-{4-[3-(2-Benzoyl-4-ethyl-phenoxy)-1-methyl-propoxy]-2-methyl-phenyl}-propionic acid

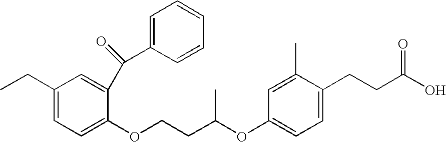

### Step A

### [5-Ethyl-2-(3-hydroxy-butoxy)-phenyl]-phenyl-methanone

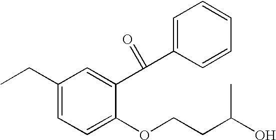

A 0° C. solution of 4-methyl-[1,3,2]dioxathiane 2,2-dioxide (0.19 g, 1.25 mmol) in ACN (10 mL) is treated with cesium carbonate (0.69 g, 2.12 mmol) and (5-ethyl-2-hydroxy-phenyl)-phenyl-methanone (0.42 g, 1.87 mmol). The mixture is stirred at ambient temperature for 10 h and concentrated in vacuo. The residue, partitioned between diethyl ether and concentrated HCl, is stirred rapidly for 10 h at ambient temperature. The resulting mixture is diluted with EtOAc, washed with water, saturated aqueous sodium bicarbonate and brine, and then dried over Na₂SO₄, and concentrated in vacuo. The crude material is purified by flash chromatography to provide 10.2 g (65%) of the title compound. ¹H NMR (400 MHz, CDCl₃) δ 7.79 (d, 2H, J=7.9 Hz), 7.54 (d, 1H, J=7.9 Hz), 7.43 (t, 2H, J=7.9 Hz), 7.29 (dd, 1H, J=8.6 Hz, 2.0 Hz), 7.23 (d, 1H, J=2.0 Hz), 6.92 (d, 1H, J=8.6 Hz), 4.12-4.06 (m, 1H, isomer 1), 4.03-3.98 (m, 1H, isomer 2), 6.34 (q, 1H, J=6.0 Hz), 2.62 (q, 2H, J=7.8 Hz), 2.04 (s, 2H), 1.64 (q, 2H, J=6.0 Hz), 1.48 (d, 1H, J=6.0 Hz), 1.22 (t, 3H, J=7.8 Hz), 1.06 (d, 3H, J=7.8 Hz). MS [EI+] 299 (M+H)⁺.

### Step B

### Methanesulfonic acid 3-(2-benzoyl-4-ethyl-phenoxy)-1-methyl-propyl ester

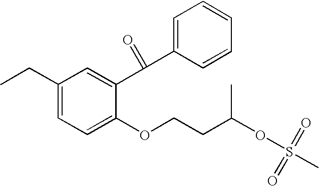

Methanesulphonyl chloride (0.1 mL, 0.60 mmol) is added to a 0° C. solution of [5-ethyl-2-(3-hydroxy-butoxy)-phenyl]-phenyl-methanone (0.15 g, 0.50 mmol) and TEA (0.11 mL, 0.75 mmol) in methylene chloride (5 mL). The resulting solution is stirred at 0° C. for 2 h and quenched with 1N HCl. The organic layer is washed with 1N HCl, dried over Na₂SO₄, and concentrated in vacuo. The crude material is purified by flash chromatography, using 20% acetone in hexanes as eluent, to provide 0.12 g (63%) of the title compound. ¹H NMR (400 MHz, CDCl₃) δ 7.78 (d, 2H, J=7.3 Hz), 7.56 (t, 1H, J=7.3 Hz), 7.44 (t, 2H, J=7.3 Hz), 7.28 (dd, 1H, J=8.4 Hz, 2.1 Hz), 7.24 (d, 1H, J=2.1 Hz), 6.88 (d, 1H, J=8.4 Hz), 4.50-4.44 (m, 1H), 4.06-3.97 (m, 1H), 3.95-3.90 (m, 1H), 2.84 (s, 3H), 2.63 (q, 2H, J=7.4 Hz), 1.87-1.79 (m, 1H), 1.72-1.64 (m, 1H), 1.21 (d, 3H, J=7.4 Hz), 1.23 (t, 3H, J=5.9 Hz). MS [EI+] 377 (M+H)⁺. R_{f}=0.25 in 33% acetone in hexanes.

### Step C

### 3-{4-[3-(2-Benzoyl-4-ethyl-phenoxy)-1-methyl-propoxy]-2-methyl-phenyl}-propionic acid methyl ester

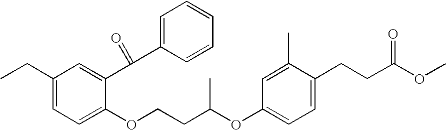

A solution of (3-(4-hydroxy-2-methyl-phenyl)-propionic acid methyl ester (0.03 g, 0.15 mmol) and methanesulfonic acid 3-(2-benzoyl-4-ethyl-phenoxy)-1-methyl-propyl ester (0.057 g, 0.151 mmol) in DMF (5 mL) is treated with cesium carbonate (0.064 g, 0.197 mmol) and heated to 50° C. under N₂. After 10 h, the reaction mixture is cooled to ambient temperature and diluted with EtOAc. The organic layer is washed with 1N HCl, water and brine, and then dried over Na₂SO₄, and concentrated in vacuo. The crude material is purified by flash chromatography, using 17% acetone in hexanes as eluent, to provide the ester. ¹H NMR (400 MHz, CDCl₃) δ 7.79 (d, 2H, J=7.3 Hz), 7.54 (t, 1H, J=7.3 Hz), 7.41 (t, 2H, J=7.3 Hz), 7.25 (d, 2H, J=7.3 Hz), 6.95 (d, 1H, J=8.1 Hz), 6.85 (d, 1H, J=8.1 Hz), 6.55 (d, 1H, J=2.4 Hz), 6.45 (dd, 1H, J=8.1 Hz, 2.4 Hz), 4.08-4.01 (m, 2H), 3.96-3.91 (m, 1H), 3.68 (s, 3H), 2.85 (t, 2H, J=7.7 Hz), 2.62 (q, 2H, J=7.4 Hz), 2.54 (t, 2H, J=7.4 Hz), 2.24 (s, 3H), 1.90-1.78 (m, 1H), 1.65-1.57 (m, 1H), 1.22 (t, 3H, J=7.7 Hz), 1.07 (d, 3H, J=6.1 Hz). MS [EI+] 475 (M+H)⁺. R_{f}=0.38 in 30% acetone in hexanes.

### Step D

### 3-{4-[3-(2-Benzoyl-4-ethyl-phenoxy)-1-methyl-propoxy]-2-methyl-phenyl}-propionic acid

A solution of 3-{4-[3-(2-Benzoyl-4-ethyl-phenoxy)-1-methyl-propoxy]-2-methyl-phenyl}-propionic acid methyl ester (0.30 g, 063 mmol) and 5N NaOH (0.3 mL) in ethanol (3 mL) is refluxed under N₂, then cooled to ambient temperature and concentrated in vacuo. The residue is dissolved in DCM, washed with 1N HCl, dried through a Varian ChemElut cartridge, and concentrated in vacuo to provide the title compound. HRMS (ES⁺) m/z exact mass calcd for C₂₉H₃₃O₅ 461.2328, found 461.2333.

### EXAMPLE 232

### 3-{4-[3-(2-Benzoyl-4-ethyl-phenoxy)-pentyloxy]-2-methyl-phenyl}-propionic acid

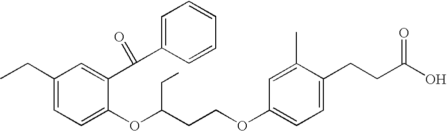

### Step A

### Toluene-4-sulfonic acid 3-hydroxy-pentyl ester

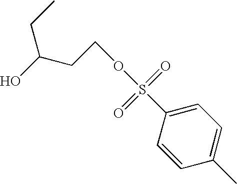

Dibutyltin oxide (0.30 g, 1.20 mmol) and TEA (10.9 mL, 78 mmol) are added to a 0° C. solution of pentane-1,3-diol (6.24 g, 59.9 mmol) in methylene chloride (100 mL). The resulting solution is treated with p-toluenesulphonic anhydride (1.14 g, 59.9 mmol) in two portions over 10 minutes. The mixture is stirred under N₂ for 10 h while gradually warning to ambient temperature. The reaction is quenched with 1N HCl. The organic layer is washed with 1N HCl, dried over Na₂SO₄, and concentrated in vacuo. The crude material is purified by flash chromatography, using 17% acetone in hexanes as eluent, to provide 1.03 g (7%) of the title compound. ¹H NMR (400 MHz, CDCl₃) δ 7.78 (d, 2H, J=8.2 Hz), 7.33 (d, 2H, J=8.2 Hz), 4.27-4.21 (m, 1H), 4.14-4.09 (m, 1H), 3.66-3.61 (m, 1H), 2.43 (s, 3H), 1.88-1.80 (m, 2H), 1.67-1.59 (m, 1H), 1.47-1.39 (m, 2H), 0.90 (t, 3H, J=7.8 Hz). R_{f}=0.15 in 33% acetone in hexanes.

### Step B

### 3-[4-(3-Hydroxy-pentyloxy)-2-methyl-phenyl]-propionic acid methyl ester

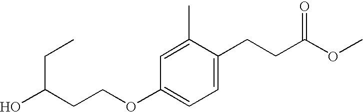

A solution of toluene-4-sulfonic acid 3-hydroxy-pentyl ester (1.03 g, 3.99 mmol) and 3-(4-hydroxy-2-methyl-phenyl)-propionic acid methyl ester (0.52 g, 2.7 mmol) in DMF (25 mL) is treated with cesium carbonate (1.47 g, 4.52 mmol) and heated to 50° C. under N₂. After 10 h, the reaction mixture is cooled to ambient temperature and diluted with diethyl ether and 1N HCl. The organic layer is washed with 1N HCl and brine, dried over Na₂SO₄, and concentrated in vacuo. The crude material is purified by flash chromatography, using 20% acetone in hexanes as eluent, to provide 0.29 (39%) of the title compound. ¹H NMR (400 MHz, CDCl₃) δ 7.02 (d, 1H, J=8.4 Hz), 6.71 (d, 1H, J=2.4 Hz), 6.67 (dd, 1H, J=8.4 Hz, 2.4 Hz), 4.16-4.10 (m, 1H, isomer 1), 4.09-4.03 (m, 1H, isomer 2), 3.81-3.76 (m, 1H), 3.66 (s, 3H), 2.86 (t, 2H, J=8.6 Hz), 2.53 (t, 2H, J=8.2 Hz), 2.36 (d, 1H, J=4.3 Hz) 2.27 (s, 3H), 1.97-1.90 (m, 1H, isomer 1), 1.88-1.80 (m, 1H, isomer 2), 1.53 (p, 2H, J=7.5 Hz), 0.96 (t, 3H, J=7.5 Hz). R_{f}=0.31 in 33% acetone in hexanes.

### Step C

### 3-[4-(3-Methanesulfonyloxy-pentyloxy)-2-methyl-phenyl]-propionic acid methyl ester

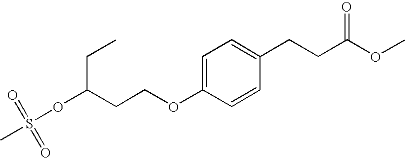

Methanesulphonyl chloride (0.1 mL, 1.3 mmol) is added to a 0° C. solution of 3-[4-(3-hydroxy-pentyloxy)-2-methyl-phenyl]-propionic acid methyl ester (0.29 g, 1.0 mmol) and TEA (0.2 mL, 1.6 mmol) in methylene chloride (10 mL). The resulting solution is stirred under N₂ for 2 h while gradually warning to ambient temperature, which then quenched with 1N HCl. The organic layer is washed with 1N HCl, dried over Na₂SO₄, and concentrated in vacuo to provide 0.37 g (100%) of the title compound. ¹H NMR (400 MHz, CDCl₃) δ 7.03 (d, 1H, J=8.4 Hz), 6.70 (d, 1H, J=2.7 Hz), 6.66 (dd, 1H, J=8.4 Hz, 2.7 Hz), 4.91 (p, 1H, J=5.8 Hz), 4.04 (t, 2H, J=5.8 Hz), 3.67 (s, 3H), 2.95 (s, 3H), 2.87 (t, 2H, J=7.4 Hz), 2.54 (t, 2H, J=7.4 Hz), 2.28 (s, 3H), 2.15-2.10 (m, 2H), 1.90-1.79 (m, 2H), 1.02 (t, 3H, J=7.4 Hz). MS [EI+] 376 (M+NH₄)⁺. R_{f}=0.30 in 33% acetone in hexanes.

### Step D

### 3-{4-[3-(2-Benzoyl-4-ethyl-phenoxy)-pentyloxy]-2-methyl-phenyl}-propionic acid

A solution of 3-[4-(3-methanesulfonyloxy-pentyloxy)-2-methyl-phenyl]-propionic acid methyl ester (0.11 g, 0.30 mmol) and (5-ethyl-2-hydroxy-phenyl)-phenyl-methanone (0.045 g, 0.20 mmol) in DMF (5 mL) is treated with cesium carbonate (0.1 g, 0.34 mmol) and heated to 50° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography, using 17% acetone in hexanes as eluent, to provide the ester. R_{f}=0.38 in 33% acetone in hexanes.

A solution of 3-{4-[3-(2-benzoyl-4-ethyl-phenoxy)-pentyloxy]-2-methyl-phenyl}-propionic acid methyl ester and 5N NaOH (0.3 mL) in ethanol (3 mL) is refluxed under N₂, then cooled to ambient temperature and concentrated in vacuo. The residue is dissolved in DCM, washed with 1N HCl, dried through a Varian ChemElut cartridge, and concentrated in vacuo to provide the title compound. ¹H NMR (400 MHz, CDCl₃) δ 7.78 (d, 2H, J=7.0 Hz), 7.51 (t, 1H, J=7.0 Hz), 7.40 (t, 2H, J=7.0 Hz), 7.23 (d, 2H, J=8.4 Hz), 7.01 (dd, 1H, J=8.4 Hz, 3.5 Hz), 6.90 (d, 1H, J=8.4 Hz), 6.61 (d, 1H, J=2.8 Hz), 6.55 (dd, 1H, J=8.4 Hz, 2.8 Hz), 4.42 (p, 1H, J=5.4 Hz), 3.76-3.71 (m, 2H), 2.90-2.85 (m 2H), 2.61 (q, 4H, J=7.6 Hz), 2.28 (s, 3H), 1.89-1.77 (m, 2H), 1.56-1.49 (m, 2H), 1.27-1.20 (m 4H), 0.75 (t, 3H, J=7.5 Hz). MS [EI+] 475 (M+H)⁺.

### EXAMPLE 233

### 3-{4-[3-(2-Benzoyl-4-trifluoromethoxy-phenoxy)-pentyloxy]-2-methyl-phenyl}-propionic acid

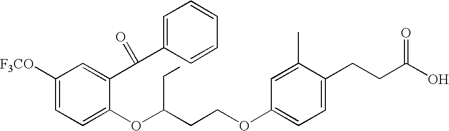

A solution of 3-[4-(3-methanesulfonyloxy-pentyloxy)-2-methyl-phenyl]-propionic acid methyl ester (0.11 g, 0.30 mmol) and (2-hydroxy-5-trifluoromethoxy-phenyl)-phenyl-methanone (0.045 g, 0.20 mmol) in DMF (5 mL) is treated with cesium carbonate (0.1 g, 0.34 mmol) and heated to 50° C. under N₂. After 10 h, the mixture is cooled to ambient temperature and diluted with diethyl ether. The organic layer is washed with 1N HCl, water and brine, and then dried over Na₂SO₄ and concentrated in vacuo. The crude material is purified by flash chromatography, using 17% acetone in hexanes as eluent, to provide the ester. R_{f}=0.38 in 33% acetone in hexanes.

A solution of 3-{4-[3-(2-benzoyl-4-trifluoromethoxy-phenoxy)-pentyloxy]-2-methyl-phenyl}-propionic acid methyl ester and 5N NaOH (0.3 mL) in ethanol (3 mL) is refluxed under N₂, and then cooled to ambient temperature and concentrated in vacuo. The residue is dissolved in DCM, washed with 1N HCl, dried through a Varian ChemElut cartridge, and concentrated in vacuo to provide the title compound. ¹H NMR (400 MHz, CDCl₃) δ 7.77 (d, 2H J=7.0 Hz), 7.55 (t, 1H, J=7.0 Hz, 1.6 Hz), 7.41 (t, 2H, J=7.8 Hz), 7.25 (d, 2H, J=2.3 Hz), 7.24 (s, 1H), 7.01 (td, 2H, J=7.0 Hz, 2.3 Hz), 6.61 (d, 1H, J=2.3), 6.56 (dd, 1H, J=7.8 Hz, 2.3 Hz), 4.47 (p, 1H, J=5.6 Hz), 3.78-3.72 (m, 2H), 2.90-2.85 (m, 2H), 2.61 (t, 1H, J=7.5 Hz), 2.28 (s, 3H), 2.17 (s, 1H), 1.92-1.80 (m, 2H), 1.55 (p, 2H, J=5.6 Hz), 1.25 (t, 1H, J=7.5 Hz), 0.76 (t, 3H, J=7.5 Hz). MS [EI+] 531 (M+H)⁺.

### EXAMPLE 234

### 3-{4-[3-(3-Benzoyl-naphthalen-2-yloxy)-butoxy]-2-methyl-phenyl}-propionic acid

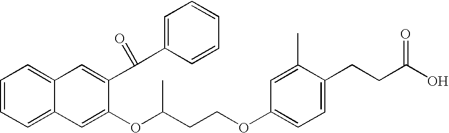

### Step A

### Acetic acid 3-hydroxy-butyl ester

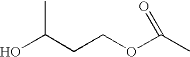

DIPEA (39.0 mL, 223 mmol) is added to a −6° C. solution of 1,3-butanediol (10.0 mL, 112 mmol) in dry methylene chloride (80 mL). Acetyl chloride (9.5 mL, 134 mmol) is added slowly via syringe to the resulting solution. The mixture is stirred at 0° C. under N₂ until all 1,3-butanediol is consumed and quenched with 1N HCl. The organic layer is washed with 1N HCl, dried over Na₂SO₄, and concentrated in vacuo. The crude material is purified by flash chromatography, using 20% acetone in hexanes as eluent, to provide 10.35 g (72%) of the title compound. ¹H NMR (400 MHz, CDCl₃) δ 4.28-4.21 (m, 1H), 4.11-4.05 (m, 1H), 3.86-3.81 (m, 1H), 2.44 (s, 1H), 2.00 (s, 3H), 1.76-1.62 (m, 2H), 1.17 (d, 3H, J=6.2 Hz). R_{f}=0.19 in 33% acetone in hexanes.

### Step B

### Acetic acid 3-(toluene-4-sulfonyloxy)-butyl ester

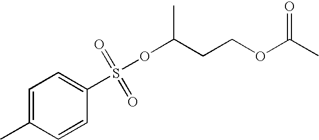

A 0° C. solution of acetic acid 3-hydroxy-butyl ester (10.4 g, 78.3 mmol), DMAP (2.87 g, 23.5 mmol), and pyridine (19.0 mL, 235 mmol) in methylene chloride (500 mL) is treated with p-toluenesulphonic anhydride (38.3 g, 117.5 mmol) and stirred at 0° C. under N₂ for 30 minutes. The mixture is warmed to ambient temperature to continue stirring until the acetic acid 3-hydroxy-butyl ester is consumed. The reaction is quenched with 1N HCl. The organic layer is washed with 1N HCl, dried over Na₂SO₄, and concentrated in vacuo. The crude material is purified by flash chromatography, using 17% acetone in hexanes as eluent, to provide 20.12 g (90%) yield of the title compound. ¹H NMR (400 MHz, CDCl₃) δ 7.79 (d, 2H, J=7.87 Hz), 7.33 (d, 2H, J=7.8 Hz), 4.76-4.71 (m, 1H), 4.05-3.99 (m, 1H), 3.93-3.87 (m, 1H), 2.44 (s, 3H), 1.96 (s, 3H), 1.94-1.80 (m, 2H), 1.34 (d, 3H, J=6.5 Hz). R_{f}=0.31 in 33% acetone in hexanes.

### Step C

### Acetic acid 3-(3-benzoyl-naphthalen-2-yloxy)-butyl ester

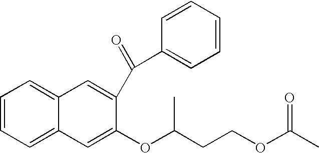

A solution of acetic acid 3-(toluene-4-sulfonyloxy)-butyl ester (2.13 g, 7.43 mmol) and (3-hydroxy-naphthalen-2-yl)-phenyl-methanone (1.23 g, 4.95 mmol) in DMF (20 mL) is treated with cesium carbonate (4.12 g, 12.6 mmol) and heated to 50° C. under N₂. After 10 h, the mixture is cooled to ambient temperature and diluted with diethyl ether. The organic layer is washed with 1N HCl, water and brine, and then dried over Na₂SO₄ and concentrated in vacuo. The crude material is purified by flash chromatography, using 14% acetone in hexanes as eluent, to provide 1.25 g (70%) of the title compound. ¹H NMR (400 MHz, CDCl₃) δ 7.89 (s, 1H), 7.80 (dd, 3H, J=8.3 Hz, 3.2 Hz), 7.75 (d, 1H, J=7.75 Hz), 7.53 (p, 2H, J=8.3 Hz), 7.44-7.36 (m, 3H), 7.20 (s, 1H), 4.62 (q, 1H, J=5.7 Hz), 3.93 (p, 1H, J=5.7 Hz), 3.86-3.80 (m, 1H), 2.01 (s, 3H), 1.75 (q, 2H, J=5.7 Hz), 1.23 (d, 3H, J=5.7 Hz). HRMS (ES⁺) m/z exact mass calcd for C24H25O4S 409.1474, found 409.1486. MS [EI+] 363 (M+H)⁺. R_{f}=0.39 in 33% acetone in hexanes.

### Step D

### [3-(3-Hydroxy-1-methyl-propoxy)-naphthalen-2-yl]-phenyl-methanone

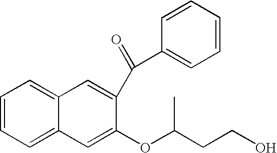

A solution of acetic acid 3-(3-benzoyl-naphthalen-2-yloxy)-butyl ester in methanol (30 mL) is treated with DIPEA (6 mL, 0.34 mmol) and stirred under N₂ for 20 h, then concentrated in vacuo. The residue is dissolved in methanol, treated with potassium carbonate (2.25 g, 0.16 mmol), and stirred at ambient temperature under N₂ for 2 h. The mixture is quenched with 1N HCl and diluted with methylene chloride. The organic layer is washed with 1N HCl, dried over Na₂SO₄, and concentrated in vacuo. The crude material is purified by flash chromatography, using 17% acetone in hexanes as eluent, to provide 0.873 g (79%) of the title compound. MS [EI+] 321 (M+H)⁺. R_{f}=0.27 in 33% acetone in hexanes.

### Step E

### Methanesulfonic acid 3-(3-benzoyl-naphthalen-2-yloxy)-butyl ester

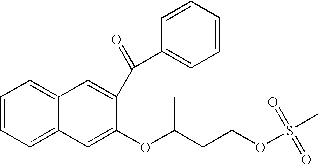

Methanesulphonyl chloride (0.3 mL, 3.3 mmol) is added to a 0° C. solution of [3-(3-hydroxy-1-methyl-propoxy)-naphthalen-2-yl]-phenyl-methanone (0.873 g, 2.72 mmol) and TEA (0.6 mL, 4.1 mmol) in methylene chloride (10 mL). The resulting is stirred under N₂ for 2 h while gradually warming to ambient temperature, which then quenched with 1N HCl. The organic layer is washed with 1N HCl, dried over Na₂SO₄, and concentrated in vacuo to provide 1.1 g (100%) of the title compound. ¹H NMR (400 MHz, CDCl₃) δ 7.85 (s, 1H), 7.83-7.76 (m, 4H), 7.57 (tt, 1H, J=7.0 Hz, 2.1 Hz), 7.52 (td, 1H, J=7.0 Hz, 1.2 Hz), 7.44 (t, 2H, J=7.8 Hz), 7.23 (s, 1H), 4.75-4.68 (m, 1H), 4.10, 4.08 (AB_{q}, 2H, J=6.1 Hz), 2.86 (s, 3H), 1.99-1.91 (m, 1H), 1.85-1.77 (m 1H), 1.29 (d, 3H, J=6.1 Hz). MS [EI+] 399 (M+H)⁺.

### Step F

### 3-{4-[3-(3-Benzoyl-naphthalen-2-yloxy)-butoxy]-2-methyl-phenyl}-propionic acid

A solution of methanesulfonic acid 3-(3-benzoyl-naphthalen-2-yloxy)-butyl ester (0.057 g, 0.14 mmol) and 3-(4-hydroxy-2-methyl-phenyl)-propionic acid methyl ester (0.036 g, 0.19 mmol) in DMF (3 mL) is treated with cesium carbonate (0.070 g, 0.21 mmol) and heated to 50° C. under N₂. After 10 h, the mixture is treated with 5N NaOH (1 mL), heated at 50° C. for 20 minutes, and cooled to ambient temperature over 2 h. The mixture is diluted with diethyl ether, washed with 1N HCl, dried through a Varian ChemElut cartridge, and concentrated in vacuo. The crude material is purified by LCMS to provide the title compound. ¹H NMR (400 MHz, CDCl₃) δ 7.86 (s, 1H), 7.79 (dd, 3H, J=8.4 Hz, 1.5 Hz), 7.69 (d, 1H, J=7.7 Hz), 7.55-7.47 (m, 2-H), 7.40-7.35 (m, 3H), 7.21 (s, 1H), 7.01 (d, 1H, J=8.4 Hz), 6.61 (d, 1H, J=2.3 Hz), 6.55 (dd, 1H, J=7.7 Hz, 2.3 Hz), 4.78 (q, 1H, J=6.0 Hz), 3.74 (t, 2H, J=6.0 Hz), 2.87 (t, 2H, J=8.4 Hz), 2.59 (t, 2H, J=8.4 Hz), 2.26 (s, 3H), 1.88 (q, 2H, J=6.0 Hz), 1.26 (d, 3H, J=6.0 Hz). HRMS (ES⁺) m/z exact mass calcd for C₃₁H₃₁O₅ 483.2171, found 483.2184.

### EXAMPLE 235

### 3-{4-[3-(3-Benzoyl-naphthalen-2-yloxy)-butylsulfanyl]-2-methyl-phenyl}-propionic acid

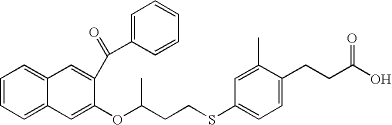

A solution of methanesulfonic acid 3-(3-benzoyl-naphthalen-2-yloxy)-butyl ester (0.057 g, 0.14 mmol) and 3-(4-mercapto-2-methyl-phenyl)-propionic acid methyl ester (0.039 g, 0.19 mmol) in DMF (3 mL) is treated with cesium carbonate (0.070 g, 0.21 mmol) and heated to 50° C. under N₂. After 10 h, the mixture is treated with 5N NaOH (1 mL), heated at 50° C. for 20 minutes and cooled to ambient temperature over 2 h. The mixture is diluted with diethyl ether, washed with 1N HCl, dried through a Varian ChemElut cartridge, and concentrated in vacuo. The crude material is purified by LCMS to provide the title compound. ¹H NMR (400 MHz, CDCl₃) δ 7.87 (s, 1H), 7.81-7.76 (m, 3H), 7.71 (d, 1H, J=7.5 Hz), 7.52 (q, 2H, J=7.5 Hz), 7.41-7.36 (m, 3H), 7.13 (s, 1H), 7.04 (s, 1H), 6.99, 6.97 (AB_{q}, 2H, J=8.0 Hz), 4.70-4.64 (m, 1H), 2.82 (t, 2H, J=7.4 Hz), 2.73-2.59 (m, 2H), 2.56 (t, 2H, J=7.4 Hz), 2.21 (s, 3H), 1.80-1.69 (m, 2H), 1.20 (d, 3H, J=7.4 Hz). HRMS (ES⁺) m/z exact mass calcd for C₃₁H₃₁O₄S 499.1943, found 499.1954.

### EXAMPLE 236

### {4-[3-(3-Benzoyl-naphthalen-2-yloxy)-butoxy]-2-methyl-phenylsulfanyl}-acetic acid

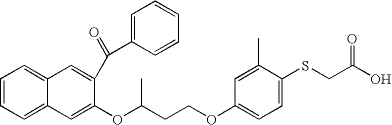

The title compound is prepared according to the procedure described in Example 235 by using methanesulfonic acid 3-(3-benzoyl-naphthalen-2-yloxy)-butyl ester (0.060 g, 0.15 mmol) and (4-hydroxy-2-methyl-phenylsulfanyl)-acetic acid ethyl ester (0.044 g, 0.19 mmol). ¹H NMR (400 MHz, CDCl₃) 37.85 (s, 1H), 7.79 (d, 3H, J=7.5 Hz), 7.70 (d, 3H, J=8.3 Hz), 7.51 (q, 2H, J=7.5 Hz), 7.39 (m, 5H), 7.21 (s, 1H), 6.66 (d, 1H, J=2.5 Hz), 6.56 (dd, 1H J=8.3 Hz, 2.5 Hz), 4.77 (p, 1H, J=5.9 Hz), 3.77-3.68 (m, 2H), 3.47 (s, 3H), 2.41 (s, 3H), 1.91-1.86 (m, 2H), 1.27 (d, 3H, J=5.9 Hz). HRMS (ES⁺) m/z exact mass calcd for C₃₀H₂₉O₅S 501.1736, found 501.1755.

### EXAMPLE 237

### {4-[3-(3-Benzoyl-naphthalen-2-yloxy)-butylsulfanyl]-2-methyl-phenoxy}-acetic acid

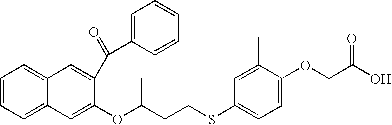

The title compound is prepared according to the procedure described in Example 235 by using methanesulfonic acid 3-(3-benzoyl-naphthalen-2-yloxy)-butyl ester (0.059 g, 0.15 mmol) and (4-mercapto-2-methyl-phenoxy)-acetic acid ethyl ester (0.044 g, 0.19 mmol). ¹H NMR (400 MHz, CDCl₃) δ 7.86 (s, 1H), 7.79 (t, 3H, J=8.6 Hz), 7.71 (d, 1H, J=8.6 Hz), 7.51 (qd, 2H, J=8.6 Hz, 1.7 Hz), 7.41-7.36 (m, 3H), 7.11 (d, 2H, J=1.7 Hz), 7.05 (dd, 1H, J=8.6 Hz, 1.7 Hz), 6.53 (d, 1H, J=8.6 Hz), 4.67-4.60 (m, 1H), 4.54 (s, 2H), 2.68-2.54 (m, 2H), 2.18 (s, 3H), 1.78-1.64 (m, 2H), 1.19 (d, 3H, J=6.0 Hz). HRMS (ES+) m/z exact mass calcd for C₃₀H₂₉O₅S 501.1736, found 501.1754.

### EXAMPLE 238

### 3-{4-[3-(2-Benzoyl-4-trifluoromethoxy-phenoxy)-butoxy]-2-methyl-phenyl}-propionic acid

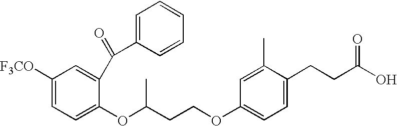

### Step A

### Acetic acid 3-(2-benzoyl-4-trifluoromethoxy-phenoxy)-butyl ester

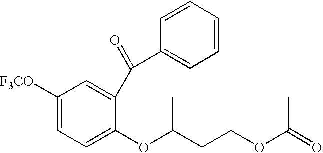

A solution of acetic acid 3-(toluene-4-sulfonyloxy)-butyl ester (1.57 g, 5.47 mmol) and (2-hydroxy-5-trifluoromethoxy-phenyl)-phenyl-methanone(1.03 g, 3.65 mmol) in DMF (15 mL) is treated with cesium carbonate (2.02 g, 6.21 mmol) and heated to 5° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography, using 17% acetone in hexanes as eluent, to provide 1.31 g (90%) of the title compound. ¹H NMR (400 MHz, CDCl₃) δ 7.75 (dd, 2H, J=8.5 Hz, 1.4 Hz), 7.55 (tt, 1H, J=7.5 Hz, 1.4 Hz), 7.41 (t, 2H, J=8.5 Hz), 7.27 (dd, 1H, J=8.5 Hz, 2.9 Hz), 7.23 (d, 1H, J=2.9 Hz), 6.95 (d, 1H, J=8.5 Hz), 4.44 (q, 1H, J=6.4 Hz), 3.92-3.86 (m, 1H), 3.82-3.76 (m, 1H), 1.97 (s, 3H), 1.67 (qd, 2H, J=6.4 Hz, 1.3 Hz), 1.14 (d, 3H, J=6.4 Hz). MS [EI+] 397 (M+H)⁺. R_{f}=0.39 in 33% acetone in hexanes.

### Step B

### [2-(3-Hydroxy-1-methyl-propoxy)-5-trifluoromethoxy-phenyl]-phenyl-methanone

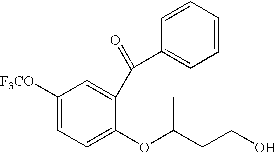

A solution of acetic acid 3-(2-benzoyl-4-trifluoromethoxy-phenoxy)-butyl ester (1.31 g, 3.31 mmol) in methanol (15 mL) is treated with potassium carbonate (2.15 g, 6.61 mmol). The mixture is stirred at ambient temperature under N₂ for 2 h, quenched with 1N HCl, and diluted with methylene chloride. The organic layer is washed with 1N HCl, dried over Na₂SO₄, and concentrated in vacuo. The crude material is purified by flash chromatography, using 25% acetone in hexanes as eluent, to provide 1.05 g (90%) of the title compound. ¹H NMR (400 MHz, CDCl₃) δ 7.75 (d, 2H, J=8.7 Hz), 7.55 (td, 1H, J=7.7 Hz, 1.0 Hz), 7.41 (t, 2H, J=7.7 Hz), 7.27 (dd, 1H, J=8.7 Hz, 2.9 Hz), 7.23 (d, 1H, J=2.9 Hz), 7.02 (d, 1H, J=8.7 Hz), 4.60-4.52 (m, 1H), 3.49 (td, 2H, J=5.6 Hz, 1.9 Hz), 2.51 (s, 1H), 1.64 (q, 2H, J=5.6 Hz), 1.15 (d, 3H, J=5.6 Hz). MS [EI+] 355 (M+H)⁺. R_{f}=0.24 in 33% acetone in hexanes.

### Step C

### Methanesulfonic acid 3-(2-benzoyl-4-trifluoromethoxy-phenoxy)-butyl ester

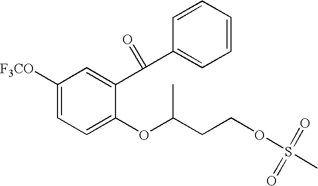

Methanesulphonyl chloride (0.28 mL, 3.6 mmol) is added to a 0° C. solution of [2-(3-hydroxy-1-methyl-propoxy)-5-trifluoromethoxy-phenyl]-phenyl-methanone (1.05 g, 3.3 mmol) and TEA (0.6 mL, 4.5 mmol) in methylene chloride (10 mL). The resulting solution is stirred under N₂ for 2 h while gradually warming to ambient temperature, which then quenched with 1N HCl. The organic layer is washed with 1N HCl, dried over Na₂SO₄, and concentrated in vacuo to provide 1.3 g (100%) of the title compound. ¹H NMR (400 MHz, CDCl₃) δ 7.76 (dd, 2H, J=7.3 Hz, 1.3 Hz), 7.57 (tt, 1H, J=7.3 Hz, 1.3 Hz), 7.44 (t, 2H, J=8.6 Hz), 7.30 (dd, 1H, J=8.6 Hz, 0.9 Hz), 7.24 (d, 1H J=3.0 Hz, 0.9 Hz), 6.98 (d, 1H, J=8.6 Hz), 4.57-4.49 (m, 1H), 4.04 (d, 1H, J=5.2 Hz), 4.02 (dd, 1H, J=5.2 Hz, 1.7 Hz), 2.88 (s, 3H), 1.92-1.84 (m, 1H), 1.77-1.69 (m, 1H), 1.19 (d, 3H, J=5.6 Hz). MS [EI+] 433 (M+H)⁺. R_{f}=0.20 in 33% acetone in hexanes.

### Step D

### 3-{4-[3-(2-Benzoyl-4-trifluoromethoxy-phenoxy)-butoxy]-2-methyl-phenyl}-propionic acid

A solution of methanesulfonic acid 3-(2-benzoyl-4-trifluoromethoxy-phenoxy)-butyl ester (0.050 g, 0.12 mmol) and (3-(4-Hydroxy-2-methyl-phenyl)-propionic acid methyl ester (0.029 g, 0.15 mmol) in DMF (3 mL) is treated with cesium carbonate (0.056 g, 0.17 mmol) and heated to 50° C. under N₂. After 10 h, the reaction mixture is treated with 5N NaOH (1 mL), heated at 50° C. for 20 minutes, then cooled to ambient temperature over 2 h. The reaction mixture is diluted with diethyl ether, washed with 1N HCl, dried through a Varian ChemElut cartridge, and concentrated in vacuo. The crude material is purified by LCMS to provide the title compound. ¹H NMR (400 MHz, CDCl₃) δ 7.76 (d, 2H J=7.5 Hz, 7.55 (t, 1H, J=7.5 Hz), 7.40 (t, 2H, J=8.3 Hz), 7.28-7.25 (m, 2H), 7.02 (d, 1H, J=8.3 Hz), 6.98 (d, 1H, J=8.3 Hz), 6.59 (d, 1H J=3.0 Hz), 6.54 (dd, 1H, J=8.3 Hz, 3.0 Hz), 4.66-4.59 (m, 1H), 3.71 (t, 2H, J=6.0 Hz), 2.88 (t, 2H, J=7.6 Hz), 2.60 (t, 2H, J=7.6 Hz), 2.27 (s, 3H), 1.82 (qd, 2H, J=6.0 Hz, 2.2 Hz), 1.58 (d, 3H, J=6.0 Hz). HRMS (ES⁺) m/z exact mass calcd for C₂₈H₂₈F₃O₆ 517.1838, found 517.1818.

### EXAMPLE 239

### 3-{4-[3-(2-Benzoyl-4-trifluoromethoxy-phenoxy)-butylsulfanyl]-2-methyl-phenyl}-propionic acid

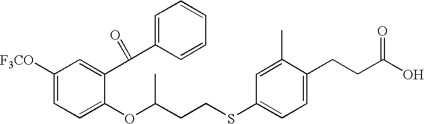

A solution of methanesulfonic acid 3-(2-benzoyl-4-trifluoromethoxy-phenoxy)-butyl ester (0.051 g, 0.12 mmol) and 3-(4-mercapto-2-methyl-phenyl)-propionic acid methyl ester (0.032 g, 0.15 mmol) in DMF (3 mL) is treated with cesium carbonate (0.0588 g, 0.18 mmol) and heated to 50° C. under N₂. After 10 h, the mixture is treated with 5N NaOH (1 mL), heated at 50° C. for 20 minutes, and cooled to ambient temperature over 2 h. The mixture is diluted with diethyl ether, washed with 1N HCl, dried through a Varian ChemElut cartridge, and concentrated in vacuo. The crude material is purified by LCMS to provide the title compound. ¹H NMR (400 MHz, CDCl₃) δ7.76 (d, 2H, J=8.1 Hz), 7.54 (tt, 1H, J=7.6 Hz), 7.41 (t, 2H, J=7.6 Hz), 7.28-7.26 (m, 2H), 7.02 (d, 2H, J=8.1 Hz), 6.98 (dd, 1H, J=8.1 Hz, 1.6 Hz), 6.91 (d, 1H, J=9.2 Hz), 4.55-4.47 (m, 1H), 2.89 (t, 2H, J=8.2 Hz), 2.71-2.53 (m, 4H), 2.25 (s, 3H), 1.75-1.61 (m, 2H), 1.13 (d, 3H, J=5.5 Hz). HRMS (ES⁺) m/z exact mass calcd for C₂₈H₂₇F₃O₅NaS 555.1429, found 555.1411.

### EXAMPLE 240

### {4-[3-(2-Benzoyl-4-trifluoromethoxy-phenoxy)-butoxy]-2-methyl-phenylsulfanyl}-acetic acid

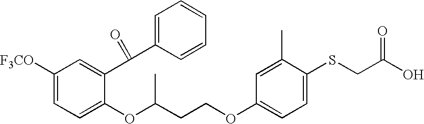

The title compound is prepared according to the procedure described in Example 239 by using methanesulfonic acid 3-(2-benzoyl-4-trifluoromethoxy-phenoxy)-butyl ester (0.054 g, 0.12 mmol) and (4-hydroxy-2-methyl-phenylsulfanyl)-acetic acid ethyl ester (0.037 g, 0.16 mmol). ¹H NMR (400 MHz, CDCl₃) δ 7.75 (d, 2H, J=7.6 Hz), 7.55 (tt, 1H, J=7.6 Hz), 7.41 (t, 2H, J=7.6 Hz), 7.29-7.24 (m, 2H), 6.97 (d, 1H, J=8.3 Hz), 6.64 (d, 1H, J=2.8 Hz), 6.54 (dd, 1H, J=8.3 Hz, 2.8 Hz), 4.64-4.57 (m, 1H), 3.75-3.66 (m, 2H), 3.48 (s, 2H), 2.42 (s, 3H), 1.83 (p, 2H, J=6.2 Hz), 1.21 (d, 3H, J=6.2 Hz). HRMS (ES+) m/z exact mass calcd for C27H26F3O6S 535.1402, found 535.1400. HRMS (ES+) m/z exact mass calcd for C₂₇H₂₅F₃O₆NaS 557.1222, found 557.1222.

### EXAMPLE 241

### {4-[3-(2-Benzoyl-4-trifluoromethoxy-phenoxy)-butylsulfanyl]-2-methyl-phenoxy}-acetic acid

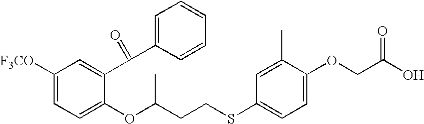

The title compound is prepared according to the procedure described in Example 239 by using methanesulfonic acid 3-(2-benzoyl-4-trifluoromethoxy-phenoxy)-butyl ester (0.064 g, 0.15 mmol) and (4-mercapto-2-methyl-phenoxy)-acetic acid ethyl ester (0.044 g, 0.19 mmol). ¹H NMR (400 MHz, CDCl₃) δ 7.74 (d, 2H, J=7.9 Hz), 7.54 (t, 1H, J=7.2 Hz), 7.41 (t, 2H, J=7.2 Hz), 7.28-7.26 (m, 2H), 7.10 (s, 1H), 7.04 (d, 1H, J=7.9 Hz), 6.90 (d, 1H, J=8.6 Hz), 6.60 (d, 1H, J=8.6 Hz), 4.66 (s, 2H), 4.52-4.47 (m, 1H), 2.65-2.58 (m, 1H), 2.55-2.48 (m, 1H), 2.22 (s, 3H), 1.71-1.57 (m, 2H), 1.11 (d, 3H, J=5.8 Hz). MS [EI+] 535 (M+H)⁺.

### EXAMPLE 242

### 3-{4-[3-(2-Benzoyl-4-ethyl-phenoxy)-1-ethyl-propoxy]-2-methyl-phenyl}-propionic acid

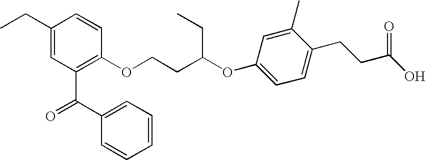

### Step A

### [5-Ethyl-2-(3-hydroxy-pentyloxy)-phenyl]-phenyl-methanone

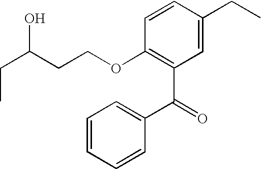

A solution of acetic acid toluene-4-sulfonic acid 3-hydroxy-pentyl ester (0.77 g, 3.0 mmol) and (5-ethyl-2-hydroxy-phenyl)-phenyl-methanone(0.45 g, 2.0 mmol) in DMF (20 mL) is treated with cesium carbonate (1.11 g, 3.4 mmol) and heated to 5° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography, using 14% acetone in hexanes as eluent, to provide 0.32 g (51%) of the title compound. ¹H NMR (400 MHz, CDCl₃) δ 7.79 (dd, 2H, J=8.7 Hz, 1.6 Hz), 7.54 (tt, 1H, J=7.6 Hz, 1.6 Hz), 7.42 (t, 2H, J=7.6 Hz), 7.28 (tt, 1H, J=8.7 Hz, 2.2 Hz), 7.23 (d, 1H, J=2.2 Hz), 6.91 (d, 1H, J=8.2 Hz), 4.11-4.06 (m, 1H), 4.03-3.98 (m, 1H), 3.32-3.26 (m, 1H), 2.61 (s, 1H), 1.68-1.51 (m, 2H), 1.38 (m, 2H), 1.21 (t, 3H, J=7.9 Hz), 0.80 (t, 3H, J=7.9 Hz). R_{f}=0.25 in 33% acetone in hexanes.

### Step B

### Methanesulfonic acid 3-(2-benzoyl-4-ethyl-phenoxy)-1-ethyl-propyl ester

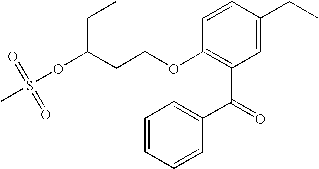

Methanesulphonyl chloride (0.14 mL, 1.8 mmol) is added to a 0° C. solution of [5-ethyl-2-(3-hydroxy-pentyloxy)-phenyl]-phenyl-methanone) (0.32 g, 1.5 mmol) and TEA (0.3 mL, 1.8 mmol) in methylene chloride (10 mL). The resulting solution is stirred under N₂ for 2 h while gradually warming to ambient temperature, which then quenched with 1N HCl. The organic layer is washed with 1N HCl, dried over Na₂SO₄, and concentrated in vacuo to provide 0.44 g (75%) of the title compound. ¹H NMR (400 MHz, CDCl₃) δ 7.80 (dd, 2H, J=7.8 Hz, 1.7 Hz), 7.55 (tt, 1H, J=7.8 Hz, 1.7 Hz), 7.43 (t, 2H, J=7.8 Hz), 7.28 (dd, 1H, J=8.4 Hz, 2.2 Hz), 7.24 (d, 1H, J=2.2 Hz), 6.88 (d, 1H, J=8.4 Hz), 4.40-4.34 (m, 1H), 4.04-3.99 (m, 1H), 3.96-3.91 (m, 1H), 2.86 (s, 3H), 2.62 (q, 2H, J=7.3 Hz), 1.88-1.69 (m, 2H), 1.54 (p, 2H, J=7.3 Hz), 1.22 (t, 3H, J=7.3 Hz), 0.77 (t, 3H, J=7.3 Hz). MS [EI+] 391 (M+H)⁺. R_{f}=0.24 in 33% acetone in hexanes.

### Step C

### 3-{4-[3-(2-Benzoyl-4-ethyl-phenoxy)-1-ethyl-propoxy]-2-methyl-phenyl}-propionic acid methyl ester

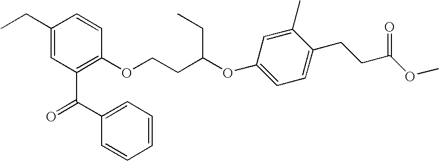

A solution of methanesulfonic acid 3-(2-benzoyl-4-ethyl-phenoxy)-1-ethyl-propyl ester (0.44 g, 1.1 mmol) and 3-(4-hydroxy-2-methyl-phenyl)-propionic acid methyl ester (0.17 g, 0.8 mmol) in DMF (10 mL) is treated with cesium carbonate (0.46 g, 1.4 mmol) and heated to 50° C. under N₂. After 10 h, the mixture is cooled to ambient temperature and diluted with diethyl ether. The organic layer is washed with 1N HCl, water and brine, and then dried over Na₂SO₄, and concentrated in vacuo. The crude material is purified by flash chromatography to provide 0.044 g (10%) of the title compound. ¹H NMR (400 MHz, CDCl₃) δ 7.80 (d, 2H, J=7.9 Hz), 7.54 (t, 1H, J=7.9 Hz), 7.42 (q, 2H, J=8.7 Hz), 7.25 (d, 2H, J=8.7 Hz), 6.94 (d, 1H, J=8.7 Hz), 6.84 (d, 1H, J=8.7 Hz), 6.57 (d, 1H, J=2.4 Hz), 4.46 (dd, 1H, J=7.9 Hz, 2.4 Hz), 4.03-3.97 (m, 1H), 3.96-3.87 (m, 2H), 3.68 (s, 3H), 2.85 (t, 2H, J=8.5 Hz), 2.62 (q, 2H, J=7.7 Hz), 2.53 (t, 2H, J=8.5 Hz), 2.23 (s, 3H), 1.83-1.73 (m, 1H), 1.71-1.61 (m, 1H), 1.51-1.36 (m, 2H), 1.23 (t, 3H, J=7.7 Hz), 0.73 (t, 3H, J=7.7 Hz). MS [EI+] 489 (M+H)⁺. R_{f}=0.03 in 33% acetone in hexanes.

### Step D

### 3-{4-[3-(2-Benzoyl-4-ethyl-phenoxy)-1-ethyl-propoxy]-2-methyl-phenyl}-propionic acid

A solution of 3-{4-[3-(2-benzoyl-4-ethyl-phenoxy)-1-ethyl-propoxy]-2-methyl-phenyl}-propionic acid methyl ester (0.044 g, 0.09 mmol) and 5N NaOH (0.4 mL) in ethanol (2 mL) is refluxed under N₂, and then cooled to ambient temperature and concentrated in vacuo. The reaction residue is dissolved in DCM, washed with 1N HCl, dried through a Varian ChemElut cartridge, and concentrated in vacuo to provide 0.01 g (24%) of the title compound. ¹H NMR (400 MHz, CDCl₃) δ 7.80 (d, 2H, J=7.9 Hz), 7.54 (t, 1H, J=7.9 Hz, 1.6 Hz), 7.42 (q, 2H, J=7.9 Hz), 7.25 (d, 2H, J=7.9 Hz), 6.95 (d, 1H, J=8.7 Hz), 6.83 (d, 1H, J=8.7 Hz), 6.57 (d, 1H, J=2.4 Hz), 6.47 (dd, 1H, J=7.9 Hz, 2.4 Hz), 4.04-3.98 (m, 1H), 3.96-3.88 (m, 2H), 2.86 (t, 2H, J=7.7 Hz), 2.62 (q, 2H, J=7.7 Hz), 2.58 (t, 2H, J=7.7 Hz), 2.23 (s, 3H), 1.83-1.74 (m, 1H), 1.70-1.62 (m, 1H), 1.49-1.38 (m, 2H), 1.22 (t, 3H, J=7.7 Hz), 0.74 (t, 3H, J=7.7 Hz). MS [EI+] 475 (M+H)⁺.

### EXAMPLE 243

### Preparation of 2-phenoxy 4-(trifluoromethyl)-phenol

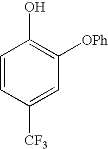

### Step A

### 4-trifluoromethyl-2-phenoxybenzaldehyde

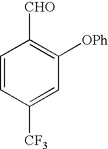

A mixture of 4-triflurormethyl-2-fluorobenzaldehyde (5 g, 26.04 mmol), phenol (2.5 g, 26.60 mmol) and K₂CO₃ (3.5 g, 26.04 mmol) in anhydrous DMF (50 mL) is warmed to 135° C., and the mixture is stirred at that temperature for 3 h. It is allowed to reach r.t. and poured into brine. The organic layer is diluted with EtOAc, washed with brine and water, and then dried, filtered and concentrated. The resulting crude residue is flash chromatographed on SiO₂ (2% EtOAc/hexanes) to afford 6.62 g of the substitution compound (96%, pale yellow solid).

### Step B

### 4-trifluorormethyl-2-phenoxyphenol

mCPBA (7 g, 70%, 28.39 mmol) is added to a solution of compound obtained in Step A (6.6 g, 24.81 mmol) in MeOH (80 mL, HPLC grade). The mixture is warmed to reflux and stirred overnight. It is allowed to reach r.t., diluted with CHCl₃ and washed with NaHSO₃ and NaHCO₃. The organic layer is dried, filtered and concentrated, affording 5.8 g of a white solid that is submitted to the next reaction without further purification. This compound is dissolved in MeOH (40 mL, HPLC grade), and HCl (2 mL, 36% solution in water) is added. The mixture is refluxed overnight, allowed to reach r.t. and poured into brine. It is extracted with EtOAc and washed with water. The organic layer is dried, filtered and concentrated to give a crude residue that is flash chromatographed on SiO₂ (3% EtOAc/hexanes) to afford 4 g of the final compound (64% for the two steps, white solid).

### EXAMPLE 244

### Preparation of 4-hydroxy-2-ethyl-phenylsulfanyl-acetic acid ethyl ester

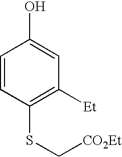

### Step A

### 3-ethylbenzyloxyphenol

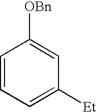

Benzyl bromide (4.92 mL, 41.36 mmol) is added to a suspension of 3-ethylphenol (5.055 g, 41.36 mmol) and K₂CO₃ (8.5 g, 61.5 mmol) in CH₃CN (50 mL, HPLC grade), and the mixture is stirred at r.t. for 5 h. The mixture is acidified with diluted HCl (1M) and partitioned between EtOAc and H₂O. The organic layer is dried, filtered and concentrated, and the product is purified by flash chromatography on SiO₂ (3% EtOAc/hexanes) to afford 8.3 g of 3-ethylbenzyloxyphenol (94%, colorless oil).

### Step B

### 4-bromo-3-ethylbenzyloxyphenol

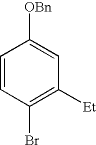

NBS (1.68 g, 9.438 mmol) is added to a solution of 3-ethylbenzyl-oxyphenol (2 g, 9.433 mmol) in CH₃CN (30 mL, HPLC grade). The mixture is stirred at r.t. overnight (c.a. 14 h) and extracted with EtOAc and H₂O. The organic layer is dried, filtered and concentrated, and the resulting crude residue is flash chromatographed on SiO₂ (2% EtOAc/hexanes) to afford 2.3 g of the bromide (84%, colorless oil).

### Step C

### 4-benzyloxy-2-ethyl-phenylsulfanyl-acetic acid ethyl ester

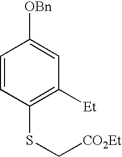

Tert-BuLi (5.25 mL, 1.7 M solution, 8.94 mmol) is added to a −78° C. cooled solution of 4-bromo-3-ethylbenzyloxyphenol (1.3 g, 4.467 mmol) in THF (20 mL). The mixture is stirred at low temperature for 30 min and allowed to reach r.t. Sulfur (150 mg, 4.68 mmol) is added in one portion, and the reaction is stirred at r.t. for 5 min. Ethylbromoacetate (2.5 mL, 22.33 mmol) is added, and the mixture is stirred at r.t. overnight (c.a. 14 h). It is quenched with NH₄Cl (sat) and extracted with EtOAc/H₂O. The organic layer is dried, filtered and concentrated, and the crude residue is flash chromatographed on SiO₂ (2˜4% EtOAc/hexanes) to afford 490 mg of the title compound (33%, colorless oil).

### Step D

### 4-hydroxy-2-ethyl-phenylsulfanyl-acetic acid ethyl ester

TiCl₄ (1.3 mL, 1 M solution in CH₂Cl₂, 1.3 mmol) is added to a −78° C. cooled solution of the benzyloxyphenol (400 mg, 1.21 mmol) in CH₂Cl₂ (12 mL), and the mixture is allowed to reach 0° C., and then r.t. and stirred for 4 h. The reaction is quenched with H₂O and diluted with CH₂Cl₂. The organic layer is washed with brine, dried, filtered and concentrated. The crude residue is flash chromatographed on SiO₂ (5-10-15% EtOAc/hexanes) to afford 160 mg of the title compound (55%, colorless oil).

### EXAMPLE 245

### Preparation of 4-hydroxy-2,6 dimethyl-dihydro-ethyl cinnamate

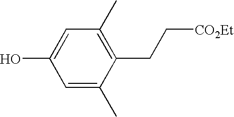

### Step A

### 3,5-dimethyl-4-bromobenzyloxyphenol

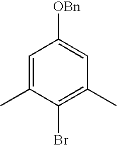

Benzyl bromide (1.53 mL, 12.86 mmol) is added to a suspension of 3,5-dimethyl-4-bromophenol (2.6 g, 12.93 mmol) and K₂CO₃ (2.2 g, 14.47 mmol) in CH₃CN (30 mL, HPLC grade). The mixture is stirred at r.t. for 16 h. It is acidified with diluted HCl (1M) and partitioned between EtOAc and H₂O. The organic layer is dried, filtered and concentrated, and the product is purified by flash chromatography on SiO₂ (5% EtOAc/hexanes) to afford 3.66 g of the benzyloxyphenol (97%, white solid).

### Step B

### 3,5-dimethyl-4-ethylacrylate-benzyloxyphenol

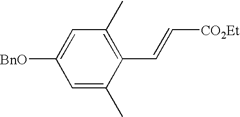

Ethyl acrylate (6 mL, 66.6 mmol) is added to a solution of 3,5-dimethyl-4-bromobenzyloxyphenol (3.6 g, 12.37 mmol), Pd(OAc)₂ (280 mg, 1.247 mmol), P(o-tol)₃ (750 mg, 2.464 mmol) and DIPEA (6 mL, 34.4 mmol) in EtCN (50 mL, HPLC grade). The mixture is warmed to 95° C. and stirred at that temperature for 36 h. It is allowed to reach r.t., filtered trough Celite and partitioned between EtOAc and H₂O. The organic layer is dried, filtered and concentrated, and the resulting crude is flash chromatographed on SiO₂ (2% EtOAc/hexanes) to afford 2.59 g of the Heck product (68%, white solid).

### Step C

### Preparation of 4-hydroxy-2,6 dimethyl-dihydro-ethyl cinnamate

Palladium (1 g, 10% on activated carbon, 0.94 mmol) is added to a solution of the benzyloxyphenol obtained in Step B (2.5 g, 8.012 mmol), and the mixture is stirred under H₂ atmosphere (H₂ balloon) overnight. The mixture is filtered trough Celite, and the solvent is removed. The crude residue is flash chromatographed on SiO₂ (10% EtOAc/hexanes) to afford 1.4 g of the title compound (79%, white solid).

### EXAMPLE 246

### Preparation of (2-hydroxy-4,5 dichloro-phenyl)-phenyl-methanone

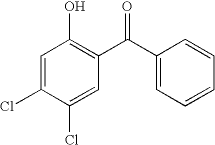

### Step A

### 3,4-dichloromethoxyphenol

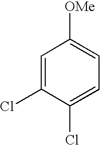

Methyl iodide (2 mL, 32.12 mmol) is added to a suspension of 3,4-dichlorophenol (2.5 g, 15.33 mmol) and K₂CO₃ (2.2 g, 15.92 mmol) in CH₃CN (40 mL, HPLC grade), and the mixture is stirred at r.t. overnight (c.a. 14 h). The mixture is poured into water, acidified with HCl (1M), and extracted with EtOAc. The organic layer is dried, filtered and concentrated, and the resulting crude residue is flash chromatographed on SiO₂ (3-5% EtOAc/hexanes) to afford 2.01 g of the methoxyphenol (74%, colorless oil).

### Step B

### (2-Methoxy-4,5 dichloro-phenyl)-phenyl-methanone

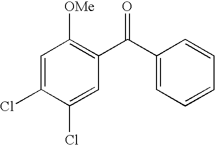

PhCOCl (1.45 mL, 12.43 mmol) is added to a 0° C. cooled solution of the methoxyphenol from Step A (2 g, 11.3 mmol) and AlCl₃ (1.81 g, 13.56 mmol) in 1,2-dichloroethane (30 mL). The mixture is stirred at that temperature for 90 min, and then at r.t. for 1 h. It is quenched with HCl (1M) and partitioned between CH₂Cl₂ and H₂O. The organic layer is dried, filtered and concentrated, and the crude residue flash chromatographed on SiO₂ (2% EtOAc/hexanes) to afford 3.15 g of the diaryl ketone (1:13 mixture of product and unreacted starting material, 11%, colorless oil).

### Step C

### Preparation of (2-hydroxy-4,5 dichloro-phenyl)-phenyl-methanone

BBr₃ (15 mL, 1 M in CH₂Cl₂ solution) is added to a −78° C. cooled solution of the methoxy compound from Step B (3.15 g of the previously described mixture) in CH₂Cl₂ (40 mL), and the mixture is allowed to reach r.t. overnight. The reaction is poured into brine and extracted with CH₂Cl₂. The organic layer is dried, filtered and concentrated, and the crude residue purified by flash chromatography on SiO₂ (2-3-10% EtOAc/hexanes) to afford 90 mg of the title compound (27%, white solid).

### EXAMPLE 247

### Preparation of 4-hydroxy-2-fluoro-dihydro-ethyl cinnamate

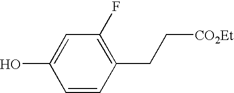

### Step A

### 3-fluorobenzyloxyphenol

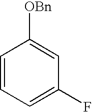

Benzyl bromide (2.9 mL, 24.08 mmol) is added to a suspension of 3-fluorophenol (3.0 g, 26.76 mmol) and K₂CO₃ (4.0 g, 28.94 mmol) in DMF (30 mL), and the mixture is stirred at r.t. for 5 h. It is acidified with diluted HCl (1M) and partitioned between EtOAc and H₂O. The organic layer is dried, filtered and concentrated, and the product is purified by flash chromatography on SiO₂ (3% EtOAc/hexanes) to afford 4.7 g of the title compound (87%, colorless oil).

### Step B

### 4-bromo-3-fluorobenzyloxyphenol

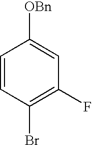

NBS (2.11 g, 11.88 mmol) is added to a solution of 3-fluorobenzyl-oxyphenol (2.4 g, 11.88 mmol) in CH₃CN (50 mL, HPLC grade). The mixture is stirred at r.t. overnight (c.a. 14 h) and extracted with EtOAc and H₂O. The organic layer is dried, filtered and concentrated, and the resulting crude residue is flash chromatographed on SiO₂ (5% EtOAc/hexanes) to afford 3.3 g of title compound (99%, white solid).

### Step C

### 3-fluoro-4-ethylacrylate-benzyloxyphenol

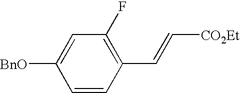

Ethyl acrylate (6.73 mL, 74.73 mmol) is added to a solution of 4-bromo-3-fluorobenzyloxyphenol (3.5 g, 12.455 mmol), Pd(OAc)₂ (280 mg, 1.245 mmol), P(o-tol)₃ (758 mg, 2.49 mmol) and DIPEA (6.5 mL, 37.37 mmol) in EtCN (80 mL, HPLC grade). The mixture is warmed to 95° C. and stirred at that temperature for 1 h. It is allowed to reach r.t., filtered trough Celite and partitioned between EtOAc and H₂O. The organic layer is dried, filtered and concentrated, and the resulting crude is flash chromatographed on SiO₂ (2-3% EtOAc/hexanes) to afford 2.05 g of the Heck product (55%, white solid).

### Step D

### Preparation of 4-hydroxy-2-fluoro-dihydro-ethyl cinnamate

Palladium (120 mg, 10% on activated carbon, 0.112 mmol) is added to a solution of the fluorobenzyloxy compound of Step C (1.2 g, 4.0 mmol), and the mixture is stirred under H₂ atmosphere (H₂ balloon) overnight (c.a. 14 h). The mixture is filtered trough Celite, and the solvent is removed in a rotatory evaporator. The crude residue is flash chromatographed on SiO₂ (10-20% EtOAc/hexanes) to afford 510 mg of the title compound (60%, colorless oil).

### EXAMPLE 248

### Preparation of 4-hydroxy-2-chloro-dihydro-ethyl cinnamate

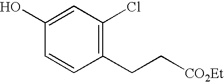

### Step A

### 4-bromo-3-chlorobenzyloxyphenol

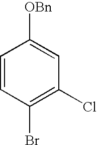

Benzyl bromide (0.83 mL, 6.95 mmol) is added to a suspension of 3-chloro-4-bromophenol (1.0 g, 4.82 mmol) and K₂CO₃ (960 mg, 6.95 mmol) in DMF (25 mL), and the mixture is stirred at r.t. for 3 h. It is acidified with diluted HCl (1M) and partitioned between Et₂O and H₂O. The organic layer is dried, filtered and concentrated, and the product is purified by flash chromatography on SiO₂ (1-2% EtOAc/hexanes) to afford 1.39 g of the title compound (97%, white solid).

### Step B

### 3-chloro-4-ethylacrylate-benzyloxyphenol

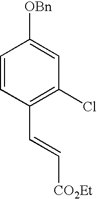

Ethyl acrylate (5.0 mL, 55.5 mmol) is added to a solution of 4-bromo-3-chlorobenzyloxyphenol (2.7 g, 9.08 mmol), palladium acetate (215 mg, 0.96 mmol), P(o-tol)₃ (550 mg, 1.8 mmol) and Et₃N (3 mL, 21.5 mmol) in EtCN (100 mL, HPLC grade). The mixture is warmed to 95° C. and stirred at that temperature overnight (c.a. 16 h). It is allowed to reach r.t., filtered trough Celite and partitioned between EtOAc and H₂O. The organic layer is dried, filtered and concentrated, and the resulting crude is flash chromatographed on SiO₂ (5% EtOAc/hexanes) to afford 1.79 g of the Heck product (62%, white solid).

### Step C

### 4-hydroxy-2-chloro-dihydro-ethyl cinnamate

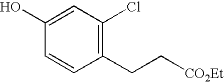

Palladium (121 mg, 10% on activated carbon, 0.113 mmol) is added to a solution of the chlorobenzyloxyphenol (1.2 g, 3.79 mmol), and the mixture is stirred under H₂ atmosphere (H₂ balloon) overnight (c.a. 14 h). The mixture is filtered trough Celite, and the solvent is removed in a rotatory evaporator. The crude residue is flash chromatographed on SiO₂ (5-10% EtOAc/hexanes), and repurified by HPLC (normal phase) to afford 515 mg of the title compound (93%, colorless oil).

### EXAMPLE 249

### Preparation of 2-(2′-pyridyl)-4-(trifluoromethyl)phenol

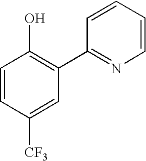

### Step A

### 2-methoxy-5-(trifluoromethyl)phenylboronic acid

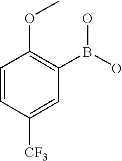

n-BuLi (1.6 M in hexane) (44.45 mL, 71.13 mmol) is added to a solution of 2-bromo-4-(trifluoromethyl)anisole (18.14 g, 71.13 mmol) in diethylether (71 mL) at −78° C., and the mixture is stirred for an hour while maintaining the internal temperature below −75° C. The mixture is stirred at r.t. for 30 minutes, cooled to −78° C. and then a solution of triisopropylborate (19.70 mL, 85.35 mmol) in diethylether (239 mL) is added. The temperature is maintained below −75° C. for an hour and then the mixture is stir at r.t. for 30 minutes. Concentrated HCl (200 mL) is added and the mixture is extracted with diethylether. The organic layers are combined, dried over sodium sulfate, filtered and concentrated under reduced pressure to give the title compound (quantitative).

### Step B

### 2-(2′-pyridyl)-4-(trifluoromethyl)anisole

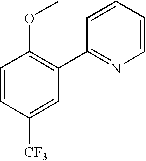

A mixture of 2-methoxy-5-(trifluoromethyl)-phenylboronic acid (15.64 g, 71.10 mmol), 2-bromopyridine (5.65 mL, 59.25 mmol), palladium tetrakis-(triphenylphosphine) (2.74 g, 2.37 mmol) and sodium carbonate (2 M in water) (83 mL, 165.9 mmol) in dimethoxyethane (118 mL) is stirred overnight under reflux. The mixture is cooled to r.t., and the layers are separated and the aqueous layer is extracted with ethylacetate. The organic layers are combined, dried, filtered and concentrated. Purification by flash chromatography, eluting with hexane:EtOAc 5:1 provides the title compound (11.68 g, 78%).

### Step C

### 2-(2′-pyridyl)-4-(trifluoromethyl)phenol

Boron tribromide (1.0 M in DCM) (92.25 mL, 92.25 mmol) is added to a solution of 2-(2′-pyridyl)-4-(trifluoromethyl)-anisole (11.68 g, 46.12 mmol) in DCM (230 mL) at −78° C., and the mixture is stirred for 10 minutes at that temperature. The bath is removed and stirred at r.t. for 1 h. Water is added slowly and stirred for 1 h, and the mixture is extracted with DCM. The organic layers are combined, dried over sodium sulfate, filtered and concentrated under reduced pressure. Purification by flash chromatography, eluting with hexane:EtOAc 5:1 provides the title compound (6.00 g, 54%).

### EXAMPLE 250

### Preparation of 4-hydroxy-2-ethyl-dihydro-ethyl cinnamate

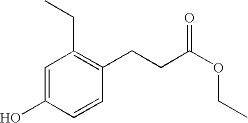

### Step A

### 3-iodobenzyloxybenzene

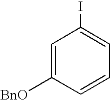

Sodium hydride (mineral dispersion 60%) (1.36 g, 34.10 mmol) is added slowly to a solution of 3-iodophenol (5.0 g, 22.73 mmol) and TABI (0.84 g, 2.27 mmol) in THF (113 mL), and the mixture is stirred overnight. The crude is treated with water and extracted with EtOAc. The organic layers are combined, dried over sodium sulfate, filtered and concentrated under reduced pressure. Purification by flash chromatography, eluting with hexane: EtOAc 10:1 provides the title compound (7.00 g, 99%). Rf=0.77 (hexane: EtOAc 5:1). ¹H NMR (200 MHz, CDCl₃): 5.03 (s, 2H), 6.93 (m, 1H), 7.02 (d, 1H, J=8.3 Hz), 7.27-7.34 (m, 7H).

### Step B

### 3-ethylbenzyloxybenzene

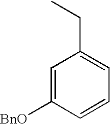

Copper (I) chloride (0.016 g, 0.17 mmol), ethyl iodide (0.40 mL, 5.03 mmol) and diethyl zinc (1.0 M, THF) (4.61 mL, 4.61 mmol) are added successively to a solution of manganese bromide (0.054 g, 0.25 mmol) in 1,3-dimethyl-3,4,5,6-tetrahydro-2(1H)pyrimidinone (4.20 mL), and the mixture is stirred at for 4 h. A solution of 3-iodobenzyloxybenzene (1.3 g, 4.19 mmol) and dichloro(diphenylphosphinoferrocene)-Pd(II) (DCM complex) (0.14 g, 0.17 mmol) in THF (21 mL) is added, and the mixture is stirred under reflux for 2.5 h. The mixture is cooled to r.t. and HCl 1N is added. The mixture is extracted with EtOAc. The organic layers are combined, dried over sodium sulfate, filtered and concentrated under reduced pressure. Purification by flash chromatography, eluting with hexane: EtOAc 20:1 provides the title compound (0.81 g, 91%). Rf=0.82 (hexane: EtOAc 5:1). ¹H NMR (200 MHz, CDCl₃): 1.30 (t, 3H, J=7.8 Hz), 2.70 (q, 2H, J=7.5 Hz), 5.11 (s, 2H), 6.86-6.91 (m, 3H), 7.23-7.53 (m, 6H).

### Step C

### 4-bromo-3-ethylbenzyloxybenzene

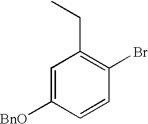

N-bromosuccinimide (0.75 g, 4.20 mmol) is added to a solution of 3-ethylbenzyloxybenzene (0.81 g, 3.82 mmol) in ACN (19 mL) and the mixture is stirred for an hour. The solvent is evaporated in vacuo and the resultant is purified by flash chromatography, eluting with hexane: EtOAc 20:1 to give the title compound (1.09 g, 98%). Rf=0.74 (hexane: EtOAc 5:1). ¹H NMR (200 MHz, CDCl₃): 1.22 (t, 3H, J=7.5 Hz), 2.72 (q, 2H, J=7.5 Hz), 5.04 (s, 2H), 6.69 (dd, 1H, J=3.0, 8.6 Hz), 6.88 (d, 2H, J=3.0 Hz), 7.32-7.45 (m, 6H).

### Step D

### 4-benzyloxy-2-ethyl-ethyl trans-cinnamate

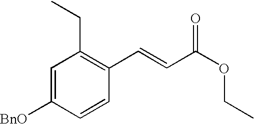

A mixture of 4-bromo-3-ethylbenzyloxybenzene (0.95 g, 3.27 mmol), palladium acetate (0.073 g, 0.33 mmol), tri-o-tolylphosphine (0.20 g, 0.65 mmol), DIPEA (1.14 mL, 6.53 mmol) and ethyl acrylate (1.42 mL, 13.06 mmol) in propionitrile (49 mL) is stirred at 90° C. under nitrogen overnight. The solution is filtered through Celite and washed with EtOAc. The mixture is concentrated under reduced pressure. Purification by flash chromatography, eluting with hexane: EtOAc 10:1 provides the title compound (0.43 g, 43%). Rf=0.22 (hexane: EtOAc 20:1). ¹H NMR (300 MHz, CDCl₃): 1.25 (t, 3H, J=7.7 Hz), 1.37 (t, 3H, J=7.1 Hz), 2.80 (q, 2H, J=7.7 Hz), 4.30 (q, 2H, J=7.3 Hz), 5.09 (s, 2H), 6.32 (d, 1H, J=15.7 Hz), 6.83-6.87 (m, 2H), 7.35-7.47 (m, 5H), 7.56 (d, 1H, J=8.5 Hz), 8.01 (d, 1H, J=15.9 Hz).

### Step E

### Preparation of 4-hydroxy-2-ethyl-dihydro-ethyl cinnamate

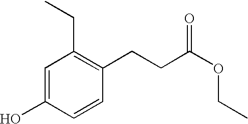

A solution of 4-benzyloxy-2-ethyl-ethyl trans-cinnamate (0.43 g, 1.39 mmol) and pd/C (10%) (0.074 g, 0.07 mmol) in methanol (14 mL) is stirred under 1 atm of hydrogen. After 4 h, the mixture is filtered through Celite and washed with metanol and concentrated under reduced pressure. Purification by flash chromatography, eluting with hexane: EtOAc 5:1 provides the title compound (0.29 g, 63%).

Rf: 0.17 (hexane: EtOAc 5:1). ¹HNMR (300 MHz, CDCl₃): 1.19 (t, 3H, J=7.5 Hz), 1.26 (t, 3H, J=7.3 Hz), 2.54-2.63 (m, 4H), 2.87-2.92 (m, 2H), 4.16 (q, 2H, J=7.1 Hz), 5.94 (s, 1H), 6.62 (dd, 1H, J=2.6, 8.3 Hz), 6.70 (d, 1H, J=2.6 Hz), 6.99 (d, 1H, J=8.3 Hz).

### EXAMPLE 251

### Preparation of 4-hydroxy-2-propyl-dihydro-ethyl cinnamate

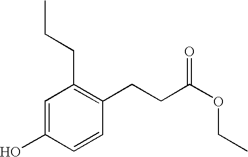

### Step A

### 3-propylbenzyloxybenzene

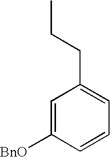

Copper (I) chloride (0.016 g, 0.17 mmol), propyl iodide (0.49 mL, 5.03 mmol) and diethyl zinc (1.0 M, THF) (4.61 mL, 4.61 mmol) is added successively to a solution of manganese bromide (0.054 g, 0.25 mmol) in 1,3-dimethyl-3,4,5,6-tetrahydro-2(1H)pyrimidinone (4.20 mL), and the mixture is stirred at r.t. for 4 h. A solution of 3-iodobenzyloxybenzene (Example 250, Step A) (1.3 g, 4.19 mmol) and dichloro-(diphenylphosphinoferrocene)palladium (II) (DCM complex) (0.14 g, 0.17 mmol) in THF (21 mL) is added, and the mixture is stirred under reflux for 2.5 h. The mixture is cooled to r.t. and 1N HCl is added. The mixture is extracted with EtOAc, and the organic layers are combined, dried over sodium sulfate, filtered and concentrated under reduced pressure. Purification by flash chromatography, eluting with hexane: EtOAc 20:1 provides the title compound together with 25% of 3-ethylbenzyloxybenzene (0.85 g, 81% overall). Rf=0.82 (hexane: EtOAc 5:1). ¹H NMR (200 MHz, CDCl₃): 1.13-1.20 (m, 3H), 1.81-1.92 (m, 2H), 2.74-2.85 (m, 2H), 5.22 (s, 2H), 7.00-7.03 (m, 3H), 7.37-7.61 (m, 6H).

### Step B

### 4-bromo-3-propylbenzyloxybenzene

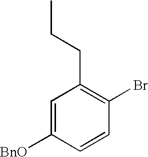

N-bromosuccinimide (0.66 g, 3.74 mmol) is added to a solution of 3-propylbenzyloxybenzene (0.85 g, 3.40 mmol) in ACN (17 mL), and the mixture is stirred for an hour. The solvent is evaporated in vacuo and purified by flash chromatography by eluting with hexane: EtOAc 20:1 to give the title compound together with 25% of 4-bromo-3-ethylbenzyl-oxybenzene (1.03 g, 99% overall). Rf=0.74 (hexane: EtOAc 5:1). ¹H NMR (200 MHz, CDCl₃): 1.00 (t, 3H, J=7.2 Hz), 1.65 (sext, 2H, J=7.2 Hz), 2.71 (q, 2H, J=7.5 Hz), 5.05 (s, 2H), 6.71 (dd, 1H, J=3.0, 8.6 Hz), 6.91 (d, 2H, J=3.0 Hz), 7.32-7.47 (m, 6H).

### Step C

### 4-benzyloxy-propylbenzaldehyde

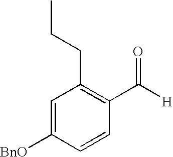

n-BuLi (1.6 M in hexane) (7.03 mL, 11.25 mmol) is added to a solution of 4-bromo-3-propylbenzyloxybenzene (2.29 g, 7.50 mmol) in THF (30 mL) under nitrogen at −78° C., and the mixture is stirred for 30 minutes. N-Formylpiperidine (1.25 mL, 11.25 mmol) is added and stirred for 4 h. The mixture is allowed to gradually warm up to −40° C., and then water is added and extracted with EtOAc. The organic layers are combined, dried and filtered, and then the solvent is evaporated in vacuo. Purification by flash chromatography by eluting with hexane: EtOAc 10:1 provides the title compound together with 25% of 4-bromo-3-ethylbenzyloxybenzene (1.00 g, 52% overall). Rf=0.63 (hexane: EtOAc 5:1). ¹H NMR (300 MHz, CDCl₃): 1.26 (t, 3H, J=7.7 Hz), 1.65 (sext, 2H, J=7.2 Hz), 2.99 (q, 2H, J=7.7 Hz), 5.13 (s, 2H), 6.84-6.94 (m, 2H), 7.33-7.46 (m, 5, H), 7.79 (d, 1H, J=8.2 Hz), 10.12 (s, 1H).

### Step D

### 4-benzyloxy-2-propyl-ethyl trans-cinnamate

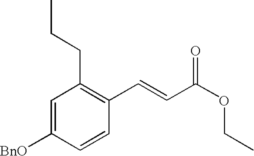

Method 1: A mixture of 4-bromo-3-ethylbenzyl-oxybenzene (0.56 g, 1.85 mmol), palladium acetate (0.042 g, 0.18 mmol), tri-o-tolylphosphine (0.11 g, 0.37 mmol), DIPEA (0.64 mL, 3.70 mmol) and ethyl acrylate (0.80 mL, 7.42 mmol) in propionitrile (28 mL) is stirred at 90° C. a under nitrogen overnight. The mixture is filtered through Celite, washed with EtOAc and concentrated under reduced pressure. Purification by flash chromatography by eluting with hexane: EtOAc 10:1 provides the title compound with a 25% of 4-benzyloxy-2-ethyl-ethyl trans-cinnamate (0.22 g, 37% overall).

Method 2: Triethylphosphono acetate (0.15 mL, 0.74 mmol) is added to a solution of 4-benzyloxy-proylbenzaldehyde (Step C) (0.16 g, 0.62 mmol) and potassium carbonate (0.26 g, 1.86 mmol) in ethanol (2.10 mL), and the mixture is stirred under reflux for 2.5 h. The mixture is cooled to r.t. and water is added. The mixture is extracted with EtOAc, and the organic layers are combined, dried and filtered. The solvent is evaporated in vacuo. Purification by flash chromatography by eluting with hexane: EtOAc 5:1 provides the title compound together with 25% of 4-benzyloxy-2-ethyl-ethyl trans-cinnamate (0.17 g, 86% overall). Rf=0.22 (hexane: EtOAc 20:1). ¹H NMR (300 MHz, CDCl₃): 0.99 (t, 3H, J=7.3 Hz), 1.25 (t, 3H, J=7.5 Hz), 1.58-1.69 (m, 2H), 2.75 (q, 2H, J=7.1 Hz), 4.29 (q, 2H, J=7.3 Hz), 5.10 (s, 2H), 6.31 (d, 1H, J=15.7 Hz), 6.85 (d, 2H, J=7.3 Hz), 7.35-7.47 (m, 5H), 7.56 (d, 1H, J=7.9 Hz), 8.00 (d, 1H, J=15.7 Hz).

### Step E

### 4-hydroxy-2-propyl-dihydro-ethyl cinnamate

A solution of 4-benzyloxy-2-propyl-ethyl trans-cinnamate (0.44 g, 1.35 mmol) and pd/C (10%) (0.14 g, 0.14 mmol) in methanol (13 mL) is stirred under 1 atm of hydrogen. After 4 h, the mixture is filtered through Celite, washed with metanol, and concentrated under reduced pressure. Purification by flash chromatography by eluting with hexane: EtOAc 5:1 provides the title compound (0.17 g, 54%) with a 25% of 4-hydroxy-2-ethyl-dihydro-ethyl cinnamate. The mixture is separated by HPLC (reverse phase purification) under acidic conditions (ACN:TFA=99.95:0.05). Rf=0.17 (hexane:EtOAc 5:1). ¹H NMR (300 MHz, CDCl₃): 0.97 (t, 3H, J=7.5 Hz), 1.26 (t, 3H, J=7.1 Hz), 1.59 (sext, 2H, J=7.5 Hz), 2.55 (q, 4H, J=8.9 Hz), 2.89 (t, 2H, J=7.5 Hz), 4.16 (q, 2H, J=7.13 Hz), 5.72 (s, 1H), 6.71 (dd, 1H, J=3.0, 8.1 Hz), 6.67 (d, 1H, J=2.6 Hz), 6.99 (d, 1H, J=8.3 Hz).

### EXAMPLE 252

### Preparation of 4-(4-hydroxy-2-methylphenyl)-butyric acid ethyl ester

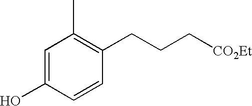

### Step A

### 4-benzyloxy-2-methyl bromobenzene

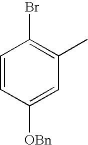

To a solution of 15 g (80.2 mmol) of 4-bromo-3-methyl-phenol and 1.5 g (10% in weight) of tetrebutylammonium iodide in THF (100 ml) is added 60% NaH (2.88 gr, 120 mmol) at 0° C. After the mixture is stirred at 0° C. for 30 min, benzyl bromide (14.3 ml 120 mmol) is added drop wise. The reaction is stirred at r.t. overnight under argon atmosphere. Then the reaction is poured into ice-water and extracted with EtOAc (3×100 ml). The organic extracts are dried over MgSO₄ and concentrated. The title compound (16.5 g, 66%) is isolated by precipitation in hexane.

### Step B

### 4-(4-benzyloxy-2-methyl-phenyl)-4-oxo-butyric acid

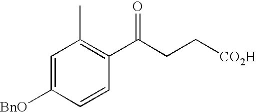

A solution of 4-benzyloxy-2-methyl bromobenzene (4 g, 14.4 mmol) in THF (25 ml) is added drop wise over a mixture of Mg (414 mg, 17.3 mmol), 1,2-dibromoethane (a few drops) and 12 (a crystal) at 70° C. under argon atmosphere. After the addition is completed, the mixture is stirred at 70° C. for 3 hours. Grignard reagent is added over a solution of succinic anhydride (1.73 gr, 17.3 mmol) and Fe(acac)₃ (254 mg, 0.7 mmol) in 25 ml of THF over argon atmosphere and is stirred overnight at r.t. The reaction is quenched with sat NH₄Cl and extracted with EtOAc (3×50 ml). The organic phase is basified with 2N NaOH, and the aqueous phase is washed with EtOAc (3×50 ml). The aqueous phase is acidified with 2N HCl and then extracted with EtOAc (3×50 ml), dried over MgSO₄ and concentrated to give 3.4 g (40%) of the title compound. The crude is used for the next step without further purification.

### Step C

### 4-(4-benzyloxy-2-methylphenyl)-4-oxo-butyric acid ethyl ester

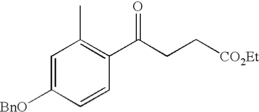

A solution of 4-(4-benzyloxy-2-methyl-phenyl)-4-oxo-butyric acid (1.6 g, 5.6 mmol) and H₂SO₄ (1 ml) in EtOH (50 ml) is stirred at 80° C. overnight. The solvent is evaporated, and water (100 ml) and sat. NaHCO₃ is added up to pH=9. The aqueous phase is extracted with EtOAc (3×50 ml) and the organics are dried over MgSO₄ and concentrated to give about 1.3 g (71%) of the title compound, which is used for the next step without further purification.

### Step, D

### 4-(4-hydroxy-2-methylphenyl)-butyric acid ethyl ester

A mixture of 4-(4-benzyloxy-2-methylphenyl)-4-oxo-butyric acid ethyl ester (1.2 g, 3.4 mmol), Pd/C (120 mg) 10% in 10 ml of AcOH is hydrogenated at 60 psi overnight. The mixture is filtered over celite, washed with EtOH and evaporated. Water (50 ml) and saturated NaHCO₃ are added until neutral pH is achieved. The aqueous phase is extracted with AcOEt (3×50 ml), and the organic phase is dried over MgSO₄ and concentrated. The crude is purificated using silica gel chromatography (hexane/EtOAc 6:1) to afford 700 mg (92%) of the title compound.

### EXAMPLE 253

### Preparation of 1,3-butanediol

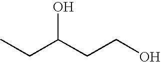

Methanol (320 ml) is added to a refluxing mixture of methyl 3-oxopentanoate (50 g, 0.38 mol) and sodium borohydride (37.8 g, 1-mol) in 800 ml of tert-butanol over a period of two hours. The mixture is to cooled to r.t. and HCl (200 ml, 6N) is added drop wise followed by K₂CO₃ (120 g) in several portions until pH is reached to 10. The solvents are evaporated in vacuo and the residue is extracted with EtOAc (2×200 ml). The mixture is filtered, and the filtrate is dried over magnesium sulfate. The solvent is evaporated in vacuo to afford 31 g of the crude product (78%). Further purification by distillation under high vacuum (b.p.=89° C./1 torr) provides about 17 g (43%) of pure 1,3-butanediol (98% HPLC-MS).

### EXAMPLE 254

### (R)-3-{6-[3-(4-Chloro-2-phenoxy-phenoxy)-butoxy]-4-methyl-pyridin-3-yl}-propionic acid

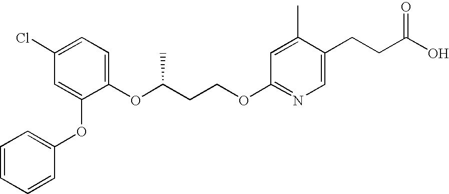

### Step A

### 2-benzyloxy-4-methylpyridine

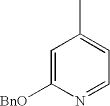

Silver carbonate (6.32 g, 22.91 mmol) and benzylbromide (6.00 mL, 50.40 mmol) are added to a solution of 2-hydroxy-4-methylpyridine (5.0 g, 45.82 mmol) in benzene (200 mL), and the mixture is stirred at 50° C. overnight. The mixture is cooled to r.t. and filtered through Celite, and the solvent is evaporated in vacuo. Purification by flash chromatography by eluting with hexane:ethyl acetate 10:1 affords the title compound (9.00 g, 98%). Rf=0.87 (hexane:ethyl acetate 1:1). ¹H NMR (300 MHz, CDCl₃): 2.30 (s, 3H), 5.37 (s, 2H), 6.64 (s, 1H), 6.72 (d, 1H, J=5.2 Hz), 7.28-7.48 (m, 5H), 8.04 (d, 1H, J=5.2 Hz).

### Step B

### 2-benzyloxy-5-bromo-4-methylpyridine

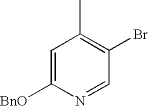

N-bromosuccinimide (5.43 g, 30.51 mmol) is added to a solution of 2-benzyloxy-4-methylpyridine (6.08 g, 30.51 mmol) in ACN (152 mL), and the mixture is stirred overnight at r.t. The solvent is evaporated in vacuo and purification by flash chromatography by eluting with hexane:ethyl acetate 10:1 affords the title compound (7.38 g, 87%). Rf=0.62 (hexane:ethyl acetate 5:1). ¹H NMR (300 MHz, CDCl₃): 2.34 (s, 3H), 5.34 (s, 2H), 6.72 (s, 1H), 7.26-7.46 (m, 5H), 8.20 (s, 1H).

### Step C

### 3-(6-Benzyloxy-4-methyl-pyridin-3-yl)-acrylic acid ethyl ester

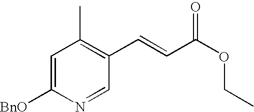

A mixture of 2-benzyloxy-5-bromo-4-methylpyridine (7.38 g, 26.53 mmol), palladium acetate (0.30 g, 1.33 mmol), tri-o-tolylphosphine (0.81 g, 2.65 mmol), diisopropylethylamine (13.9 mL, 79.59 mmol) and ethyl acrylate (11.5 mL, 106.13 mmol) in propionitrile (106 mL) is stirred 90° C. under nitrogen overnight. The mixture is filtered off through Celite, washed with ethyl acetate, and concentrated under reduced pressure. Purification by flash chromatography by eluting with hexane:ethyl acetate 10:1 affords the title compound (4.04 g, 51%) together with starting material (2.87 g, 39%). Rf=0.27 (hexane:ethyl acetate 5:1). ¹H NMR (300 MHz, CDCl₃): 1.35 (t, 3H, J=7.3 Hz), 2.38 (s, 3H), 4.28 (q, 2H, J=7.3 Hz), 5.40 (s, 2H), 6.33 (d, 1H, J=16.1 Hz), 6.65 (s, 1H), 7.29-7.46 (m, 5H), 7.82 (d, 1H, J=16.0 Hz), 8.34 (s, 1H). (m, 5H), 7.56 (d, 1H, J=7.9 Hz), 8.00 (d, 1H, J=15.7 Hz).

### Step D

### 3-(6-Hydroxy-4-methyl-pyridin-3-yl)-propionic acid ethyl ester

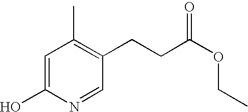

A solution of 3-(6-benzyloxy-4-methyl-pyridin-3-yl)-acrylic acid ethyl ester (5.91 g, 19.87 mmol) and palladium is stirred under 1 atm H₂ under carbon (10%) (2.11 g, 1.99 mmol) in ethanol (50 mL) and acetic acid (10 mL). After stirring overnight, the mixture is filtered through Celite, washed with methanol, and concentrated under reduced pressure. The crude product is dissolved in ethyl acetate and washed with 10% HCl. The aqueous layer is neutralized with 10% NaOH, and the title compound is precipitated from this aqueous layer, which is filtered and dried. The remaining aqueous layer is extracted with DCM, and then dried with Na₂SO₄ and filtered. The solvent is evaporated in vacuo giving a second batch of the title compound. Total amount: 2.60 g (62%). Rf=0.05 (hexane:ethyl acetate 5:1). ¹HNMR (300 MHz, CDCl₃): 1.24 (t, 3H, J=7.3 Hz), 2.21 (s, 3H), 2.49 (t, 2H, J=8.0 Hz), 2.71 (t, 2H, J=7.7 Hz), 4.16 (q, 2H, J=7.3 Hz), 6.39 (s, 1H), 7.11 (s, 1H).

### Step E

### (R)-3-{6-[3-(4-Chloro-2-phenoxy-phenoxy)-butoxy]-4-methyl-pyridin-3-yl}-propionic acid

A solution of (S)-methanesulfonic acid 3-(4-chloro-2-phenoxy-phenoxy)-butyl ester (0.177 g, 0.48 mmol) and 3-(6-hydroxy-4-methyl-pyridin-3-yl)-propionic acid ethyl ester (0.1 g, 0.48 mmol) in DMF (5 mL) is treated with cesium carbonate (171 mg, 0.53 mmol). The mixture is heated to 50° C. and stirred overnight. The mixture is then treated with aqueous 5N NaOH (0.4 mL, 2.2 mmol) and stirred for 2 additional hours at 50° C. The mixture is cooled and quenched with 1N HCl to give pH=4. The mixture is extracted with Et₂O. The organic layer is washed with brine, dried over sodium sulfate and filtered. The solvent is removed, and the crude product is purified by prep HPLC to afford 72 mg (33%) of the title product. ¹H NMR (400 MHz, CDCl₃); MS (ES⁺) m/z mass calcd for C₂₅H₂₆ClNO₅ 455, found 456 (M+1, 100%).

### EXAMPLE 255

### (R)-3-{6-[3-(4-Ethyl-2-phenoxy-phenoxy)-butoxy]-4-methyl-pyridin-3-yl}-propionic acid

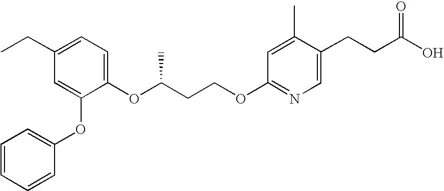

A solution of (S)-methanesulfonic acid 3-(4-ethyl-2-phenoxy-phenoxy)-butyl ester (0.174 g, 0.48 mmol) and 3-(6-hydroxy-4-methyl-pyridin-3-yl)-propionic acid ethyl ester (0.1 g, 0.48 mmol) in DMF (5 mL) is treated with cesium carbonate (171 mg, 0.53 mmol). The mixture is heated to 50° C. and stirred overnight. The mixture is treated with aqueous 5N NaOH (0.4 mL, 2.2 mmol) and stirred for 2 additional hours at 50° C. The mixture is cooled and quenched with 1N HCl to give pH=4. The mixture is extracted with Et₂O, and the organic layer is washed with brine, dried over sodium sulfate and filtered. The solvent is removed, and the crude product is purified by prep HPLC to afford 77 mg (36%) of the desired product. ¹H NMR (400 MHz, CDCl₃); MS (ES⁺) m/z mass calcd for C₂₇H₃₁NO₅ 449, found 450 (M+1, 100%).

### EXAMPLE 256

### (R)-3-{4-Methyl-6-[3-(2-phenoxy-4-trifluoromethyl-phenoxy)-butoxy]-pyridin-3-yl}-propionic acid

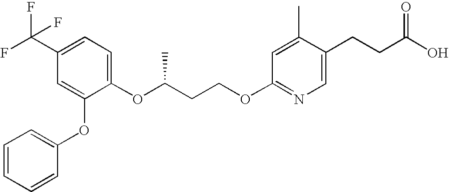

A solution of (S)-Methanesulfonic acid 3-(2-phenoxy-4-trifluoromethyl-phenoxy)-butyl ester (0.193 g, 0.48 mmol) and 3-(6-Hydroxy-4-methyl-pyridin-3-yl)-propionic acid ethyl ester (0.1 g, 0.48 mmol) in DMF (5 mL) is treated with cesium carbonate (171 mg, 0.53 mmol). The mixture is heated to 50° C. and stirred overnight. The mixture is treated with aqueous 5N NaOH (0.4 mL, 2.2 mmol) and stirred for 2 additional hours at 50° C. The mixture is cooled and quenched with 1N HCl to give pH=4. The mixture is extracted with Et₂O, and the organic layer is washed with brine, dried over sodium sulfate and filtered. The solvent is removed, and the crude product is purified by prep HPLC to afford 96 mg (41%) of the desired product. ¹H NMR (400 MHz, CDCl₃); MS (ES⁺) m/z mass calcd for C₂₆H₂₆F₃NO₅ 489, found 490 (M+1, 100%).

### EXAMPLE 257

### (R)-3-{6-[3-(2-Benzoyl-4-ethyl-phenoxy)-butoxy]-4-methyl-pyridin-3-yl}-propionic acid

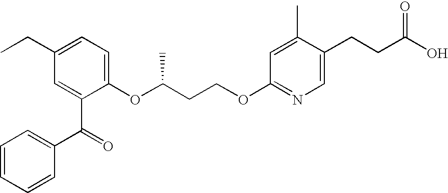

A solution of (S)-methanesulfonic acid 3-(2-benzoyl-4-ethyl-phenoxy)-butyl ester (0.18 g, 0.48 mmol) and 3-(6-hydroxy-4-methyl-pyridin-3-yl)-propionic acid ethyl ester (0.1 g, 0.48 mmol) in DMF (5 mL) is treated with cesium carbonate (171 mg, 0.53 mmol). The mixture is heated to 50° C. and stirred overnight. The mixture is treated with aqueous 5N NaOH (0.4 mL, 2.2 mmol) and stirred for 2 additional hours at 50° C. The mixture is cooled and quenched with 1N HCl to give pH=4. The mixture is extracted with Et₂O, and the organic layer is washed with brine, dried over sodium sulfate and filtered. The solvent is removed, and the crude product is purified by prep HPLC to afford 64 mg (29%) of the desired product. ¹H NMR (400 MHz, CDCl₃); MS (ES⁺) m/z mass calcd for C₂₈H₃₁NO₅ 461, found 462 (M+1, 100%).

### EXAMPLE 258

### (R)-{4-[3-(4-Ethyl-2-pyridin-2-yl-phenoxy)-butoxy]-3-methyl-phenyl}-acetic acid

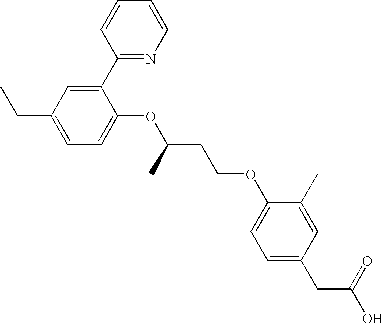

[4-(3-Methanesulfonyloxy-butoxy)-3-methyl-phenyl]-acetic acid methyl ester is reacted with 4-ethyl-2-pyridin-2-yl-phenol as in Example 11 to afford 0.115 g (43%) of (R)-{4-[3-(4-Ethyl-2-pyridin-2-yl-phenoxy)-butoxy]-3-methyl-phenyl}-acetic acid. ¹H NMR (400 MHz, CDCl₃); HRMS (ES⁺) m/z mass calcd for C₂₆H₂₉NO₄ 420.2175, found 420.2201 (M+1).

### EXAMPLE 259

### (R)-{3-Methyl-4-[3-(2-pyridin-2-yl-4-trifluoromethyl-phenoxy)-butoxy]-phenyl}-acetic acid

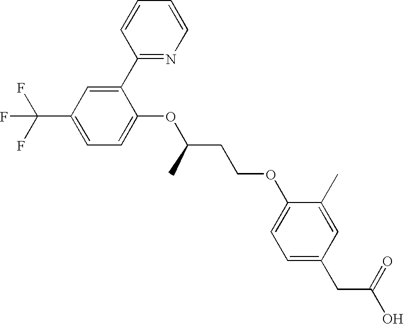

[4-(3-Methanesulfonyloxy-butoxy)-3-methyl-phenyl]-acetic acid methyl ester is reacted with 2-pyridin-2-yl-4-trifluoromethyl-phenol as in Example 11 to afford 0.118 g (61%) of (R)-{3-methyl-4-[3-(2-pyridin-2-yl-4-trifluoromethyl-phenoxy)-butoxy]-phenyl}-acetic acid. ¹H NMR (400 MHz, CDCl₃); HRMS (ES⁺) m/z mass calcd for C₂₅H₂₄NF₃O₄ 460.1736, found 460.1733 (M+1).

### EXAMPLE 260

### (R)-{3-Methyl-4-[3-(2-pyrimidin-2-yl-4-trifluoromethyl-phenoxy)-butoxy]-phenyl}-acetic acid

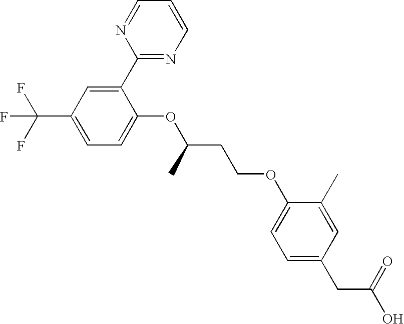

[4-(3-Methanesulfonyloxy-butoxy)-3-methyl-phenyl]-acetic acid methyl ester is reacted with 2-pyrimidin-2-yl-4-trifluoromethyl-phenol as in Example 11 to afford 0.084 g (71%) of (R)-{3-methyl-4-[3-(2-pyrimidin-2-yl-4-trifluoromethyl-phenoxy)-butoxy]-phenyl}-acetic acid. ¹H NMR (400 MHz, CDCl₃); HRMS (ES⁺) m/z mass calcd for C₂₄H₂₃N₂F₃O₄ 461.1688, found 461.1716 (M+1).

### EXAMPLE 261

### (R)-(4-{3-[4-Chloro-2-(4-fluoro-phenoxy)-phenoxy]-butoxy}-3-methyl-phenyl)-acetic acid

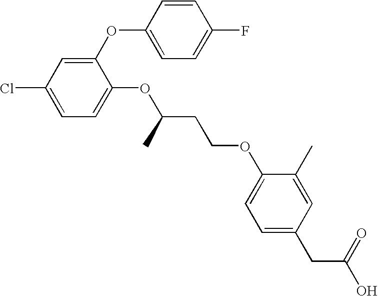

[4-(3-Methanesulfonyloxy-butoxy)-3-methyl-phenyl]-acetic acid methyl ester is reacted with 4-chloro-2-(4-fluoro-phenoxy)-phenol as in Example 11 to afford 0.172 g (56%) of (R)-(4-{3-[4-chloro-2-(4-fluoro-phenoxy)-phenoxy]-butoxy}-3-methyl-phenyl)-acetic acid. ¹H NMR (400 MHz, CDCl₃); MS (ES) m/z mass calcd for C₂₅H₂₄ClFO₅ 458, found 457 and 459 (M−1 and M+1).

### EXAMPLE 262

### (R)-{3-Methyl-4-[3-(2-o-tolyloxy-4-trifluoromethyl-phenoxy)-butoxy]-phenyl}-acetic acid

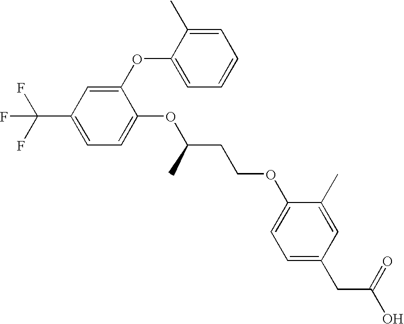

[4-(3-Methanesulfonyloxy-butoxy)-3-methyl-phenyl]-acetic acid methyl ester is reacted with 2-o-tolyloxy-4-trifluoromethyl-phenol as in Example 11 to afford 0.124 g (65%) of (R)-{3-methyl-4-[3-(2-o-tolyloxy-4-trifluoromethyl-phenoxy)-butoxy]-phenyl}-acetic acid. ¹H NMR (400 MHz, CDCl₃); MS (ES⁻) m/z mass calcd for C₂₇H₂₇F₃O₅ 488, found 487 (M−1).

### EXAMPLE 263

### (R)-{3-Methyl-4-[3-(2-phenoxy-4-trifluoromethyl-phenoxy)-butoxy]-phenyl}-acetic acid

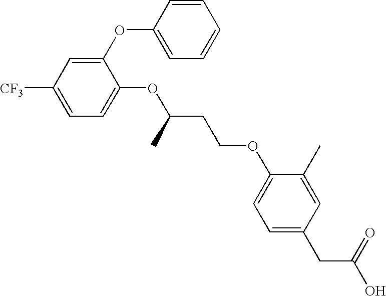

[4-(3-Methanesulfonyloxy-butoxy)-3-methyl-phenyl]-acetic acid methyl ester is reacted with 4-chloro-2-(4-fluoro-phenoxy)-phenol as in Example 11 to afford 0.230 g (58%) of (R)-{3-methyl-4-[3-(2-phenoxy-4-trifluoromethyl-phenoxy)-butoxy]phenyl}-acetic acid. ¹H NMR (400 MHz, CDCl₃); MS (ES⁻) m/z mass calcd for C₂₆H₂₅F₃O₅ 474, found 473 (M−1).

### EXAMPLE 264

### (R)-{4-[3-(2-Phenoxy-4-trifluoromethyl-phenoxy)-butoxy]-phenyl}-acetic acid

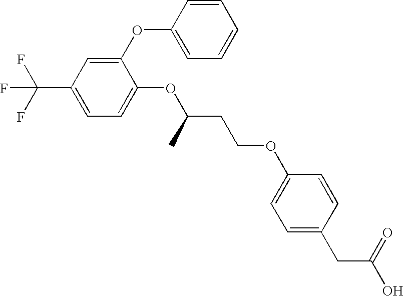

Methanesulfonic acid 3-(2-phenoxy-4-trifluoromethyl-phenoxy)-butyl ester is reacted with methyl-4-hydroxyphenylacetate as in Example 11 to afford 0.154 g (54%) of (R)-{4-[3-(2-phenoxy-4-trifluoromethyl-phenoxy)-butoxy]-phenyl}-acetic acid. ¹H NMR (400 MHz, CDCl₃); MS (ES⁻) m/z mass calcd for C₂₅H₂₃F₃O₅ 460, found 459 (M−1).

### EXAMPLE 265

### (R)-{2-Methyl-4-[3-(2-o-tolyloxy-4-trifluoromethyl-phenoxy)-butoxy]-phenylsulfanyl}-acetic acid

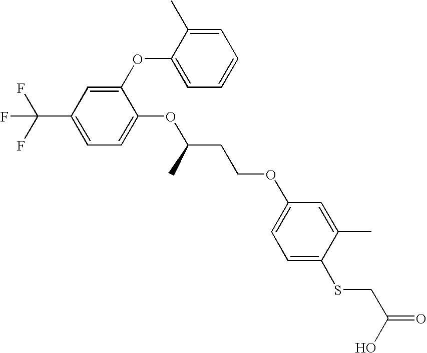

[4-(3-Methanesulfonyloxy-butoxy)-2-methyl-phenylsulfanyl]-acetic acid ethyl ester is reacted with 2-o-tolyloxy-4-trifluoromethyl-phenol as in Example 11 to afford 0.124 g (58%) of (R)-{2-methyl-4-[3-(2-o-tolyloxy-4-trifluoromethyl-phenoxy)-butoxy]-phenylsulfanyl}-acetic acid. ¹H NMR (400 MHz, CDCl₃); MS (ES⁻) m/z mass calcd for C₂₇H₂₇F₃O₅S 520, found 519 (M−1).

### EXAMPLE 266

### (R)-{2-Methyl-4-[3-(2-o-tolyloxy-4-trifluoromethyl-phenoxy)-butylsulfanyl]-phenoxy}-acetic acid

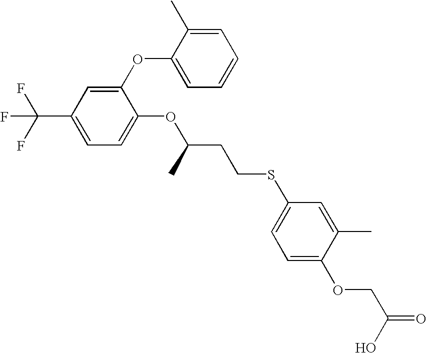

[4-(3-Methanesulfonyloxy-butylsulfanyl)-2-methyl-phenoxy]-acetic acid ethyl ester is reacted with 2-o-tolyloxy-4-trifluoromethyl-phenol as in Example 11 to afford 0.156 g (73%) of (R)-{2-methyl-4-[3-(2-o-tolyloxy-4-trifluoromethyl-phenoxy)-butylsulfanyl]-phenoxy}-acetic acid. ¹H NMR (400 MHz, CDCl₃); HRMS (ES⁺) m/z mass calcd for C₂₇H₂₇F₃O₅S 521.1609, found 521.1599 (M+1).

### EXAMPLE 267

### (R)-{2-Methyl-4-[3-(2-pyrimidin-2-yl-4-trifluoromethyl-phenoxy)-butylsulfanyl]-phenoxy}-acetic acid

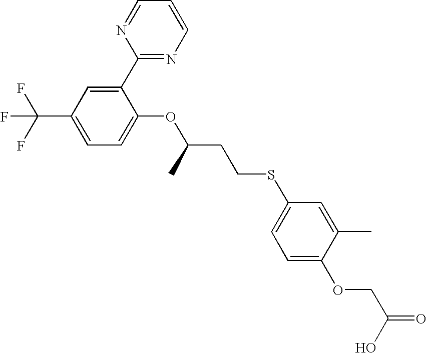

[4-(3-Methanesulfonyloxy-butylsulfanyl)-2-methyl-phenoxy]-acetic acid ethyl ester is reacted with 2-pyrimidin-2-yl-4-trifluoromethyl-phenol as in Example 11 to afford 0.050 g (51%) of (R)-{2-methyl-4-[3-(2-pyrimidin-2-yl-4-trifluoromethyl-phenoxy)-butylsulfanyl]-phenoxy}-acetic acid. ¹H NMR (400 MHz, CDCl₃); HRMS (ES⁺) m/z mass calcd for C₂₄H₂₃N₂O₄F₃S 493.1409, found 493.1407 (M+1).

### EXAMPLE 268

### (R)-(4-{3-[2-(4-Fluoro-phenoxy)-4-trifluoromethyl-phenoxy]-butylsulfanyl}-2-methyl-phenoxy)-acetic acid

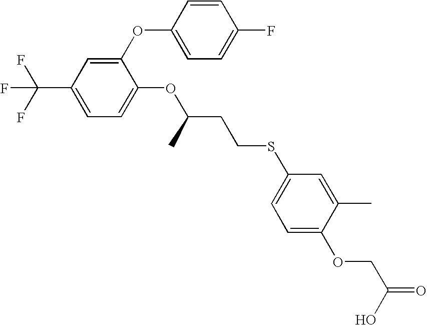

[4-(3-Methanesulfonyloxy-butylsulfanyl)-2-methyl-phenoxy]-acetic acid ethyl ester is reacted with 2-(4-fluoro-phenoxy)-4-trifluoromethyl-phenol as in Example 11 to afford 0.035 g (29%) of (R)-(4-{3-[2-(4-fluoro-phenoxy)-4-trifluoromethyl-phenoxy]-butylsulfanyl}-2-methyl-phenoxy)-acetic acid. ¹H NMR (400 MHz, CDCl₃); HRMS (ES⁺) m/z mass calcd for C₂₆H₂₄O₅F₄S 525.1359, found 525.1351 (M+1).

### EXAMPLE 269

### (R)-(4-{3-[4-Chloro-2-(4-fluoro-phenoxy)-phenoxy]-butylsulfanyl}-2-methyl-phenoxy)-acetic acid

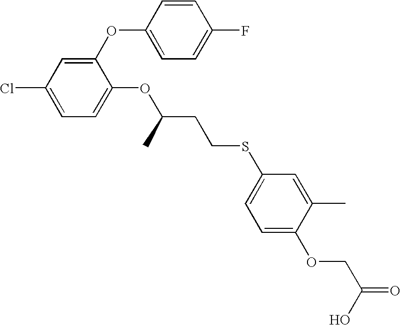

[4-(3-Methanesulfonyloxy-butylsulfanyl)-2-methyl-phenoxy]-acetic acid ethyl ester is reacted with 4-chloro-2-(4-fluoro-phenoxy)-phenol as in Example 11 to afford 0.012 g (11%) of (R)-(4-{3-[4-chloro-2-(4-fluoro-phenoxy)-phenoxy]-butylsulfanyl}-2-methyl-phenoxy)-acetic acid. ¹H NMR (400 MHz, CDCl₃); HRMS (ES⁺) m/z mass calcd for C₂₅H₂₄O₅FClS 491.1095, found 491.1106 (M+1).

### EXAMPLE 270

### 3-{4-[3-(4-Chloro-2-phenoxy-phenoxy)-2-methyl-propoxy]-2-methyl-phenyl}-propionic acid

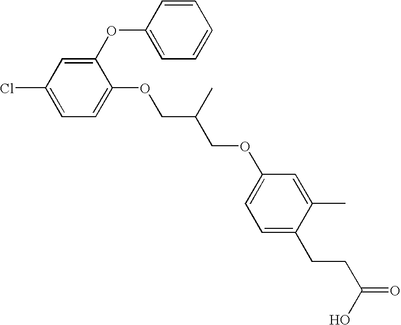

3-[2-methyl-4-(3-methanesulfonyloxy-2-methyl-propoxy)-phenyl]-propionic acid ethyl ester is reacted with 4-chloro-2-phenoxy-phenol as in Example 11 to afford 0.055 g (98%) of 3-{4-[3-(4-chloro-2-phenoxy-phenoxy)-2-methyl-propoxy]-2-methyl-phenyl}-propionic acid. ¹H NMR (400 MHz, CDCl₃); MS (ES⁻) m/z mass calcd for C₂₆H₂₇O₅Cl 454, found 453 and 455 (M−1 and M+1).

### EXAMPLE 271

### 3-{4-[3-(4-Chloro-2-phenoxy-phenoxy)-2-methyl-propoxy]-2-ethyl-phenyl}-propionic acid

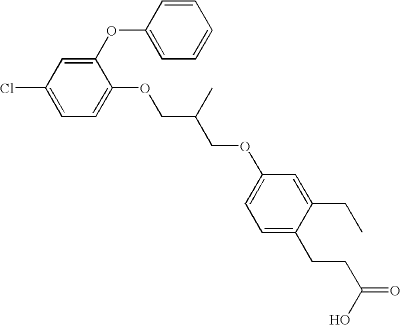

3-[2-ethyl-4-(3-methanesulfonyloxy-2-methyl-propoxy)-phenyl]-propionic acid ethyl ester is reacted with 4-chloro-2-phenoxy-phenol as in Example 11 to afford 0.250 g (56%) of 3-{4-[3-(4-chloro-2-phenoxy-phenoxy)-2-methyl-propoxy]-2-ethyl-phenyl}-propionic acid. ¹H NMR (400 MHz, CDCl₃); HRMS (ES⁺) m/z mass calcd for C₂₇H₂₉O₅Cl 469.1782, found 469.1804 (M+1).

### EXAMPLE 272

### 3-{2-Methyl-4-[2-methyl-3-(2-phenoxy-4-trifluoromethyl-phenoxy)-propoxy]-phenyl}-propionic acid

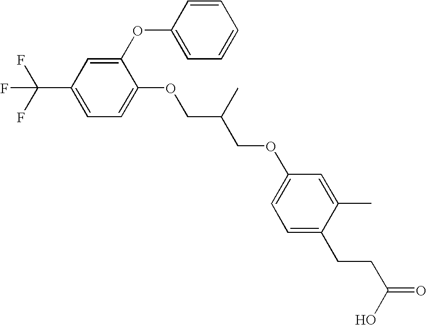

3-[2-methyl-4-(3-methanesulfonyloxy-2-methyl-propoxy)-phenyl]-propionic acid ethyl ester is reacted with 2-phenoxy-4-trifluoromethyl-phenol as in Example 11 to afford 0.052 g (96%) of 3-{2-methyl-4-[2-methyl-3-(2-phenoxy-4-trifluoromethyl-phenoxy)-propoxy]-phenyl}-propionic acid. ¹H NMR (400 MHz, CDCl₃); MS (ES⁻) m/z mass calcd for C₂₇H₂₇O₅F₃ 488, found 487 (M−1).

### EXAMPLE 273

### 3-{2-Ethyl-4-[2-methyl-3-(2-phenoxy-4-trifluoromethyl-phenoxy)-propoxy]-phenyl}-propionic acid

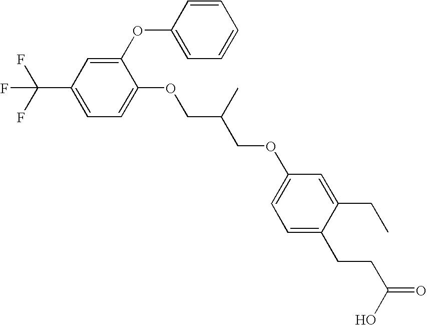

3-[2-Ethyl-4-(3-methanesulfonyloxy-2-methyl-propoxy)-phenyl]-propionic acid ethyl ester is reacted with 2-phenoxy-4-trifluoromethyl-phenol as in Example 11 to afford 0.048 g (60%) of 3-{2-ethyl-4-[2-methyl-3-(2-phenoxy-4-trifluoromethyl-phenoxy)-propoxy]-phenyl}-propionic acid. ¹H NMR (400 MHz, CDCl₃); MS (ES⁻) m/z mass calcd for C₂₈H₂₉O₅F₃ 502, found 501 (M−1).

### EXAMPLE 274

### 2-Methyl-2-{4-[2-methyl-3-(2-phenoxy-4-trifluoromethyl-phenoxy)-propoxy]-phenoxy}-propionic acid

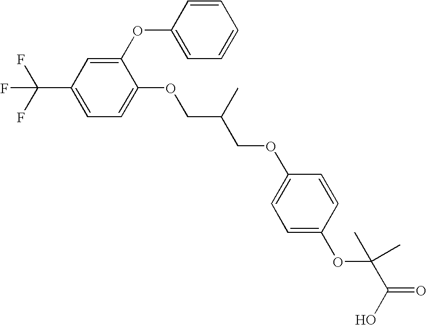

Methanesulfonic acid 2-methyl-3-(2-phenoxy-4-trifluoromethyl-phenoxy)-propyl ester is reacted with 2-(4-hydroxy-phenoxy)-2-methyl-propionic acid ethyl ester as in Example 1 to afford 0.270 g (37%) of 2-methyl-2-{4-[2-methyl-3-(2-phenoxy-4-trifluoromethyl-phenoxy)-propoxy]-phenoxy}-propionic acid. ¹H NMR (400 MHz, CDCl₃); HRMS (ES⁺) m/z mass calcd for C₂₇H₂₇O₆F₃ 503.1682, found 503.1664 (M+1).

### EXAMPLE 275

### 2-Methyl-2-{2-methyl-4-[2-methyl-3-(2-phenoxy-4-trifluoromethyl-phenoxy)-propoxy]-phenoxy}-propionic acid

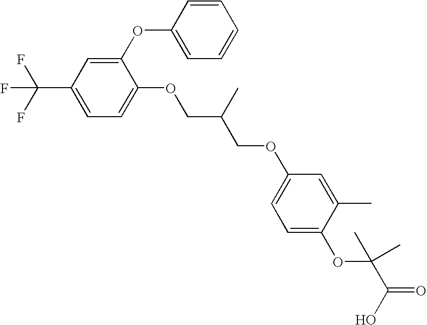

Methanesulfonic acid 2-methyl-3-(2-phenoxy-4-trifluoromethyl-phenoxy)-propyl ester is reacted with 2-(4-hydroxy-2-methyl-phenoxy)-2-methyl-propionic acid ethyl ester as in Example 1 to afford 0.165 g (60%) of 2-methyl-2-{2-methyl-4-[2-methyl-3-(2-phenoxy-4-trifluoromethyl-phenoxy)-propoxy]-phenoxy}-propionic acid. ¹H NMR (400 MHz, CDCl₃); MS (ES⁻) m/z mass calcd for C₂₈H₂₉O₆F₃ 518, found 517 (M−1).

### EXAMPLE 276

### {2-Methyl-4-[2-methyl-3-(2-phenoxy-4-trifluoromethyl-phenoxy)-propoxy]-phenoxy}-acetic acid

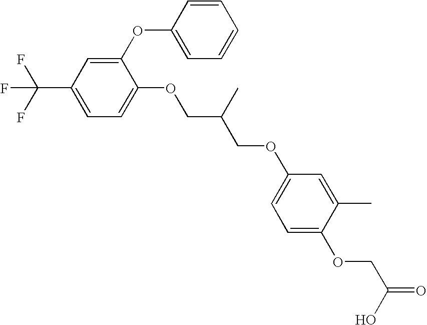

Methanesulfonic acid 2-methyl-3-(2-phenoxy-4-trifluoromethyl-phenoxy)-propyl ester is reacted with 2-(4-hydroxy-2-methyl-phenoxy)-propionic acid methyl ester as in Example 1 to afford 0.047 g (32%) of {2-methyl-2-[2-methyl-3-(2-phenoxy-4-trifluoromethyl-phenoxy)-propoxy]-phenoxy}-acetic acid. ¹H NMR (400 MHz, CDCl₃); MS (ES⁻) m/z mass calcd for C₂₆H₂₅O₆F₃ 490, found 489 (M−1).

### EXAMPLE 277

### 2-{4-[3-(2-Benzoyl-4-trifluoromethyl-phenoxy)-2-methyl-propoxy]-phenoxy}-2-methyl-propionic acid

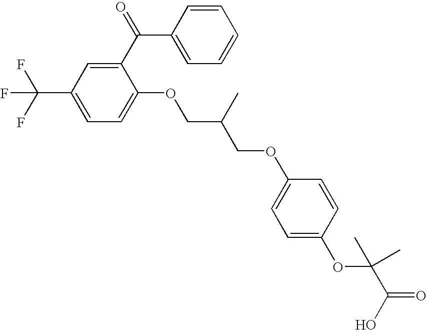

2-[4-(3-Methanesulfonyloxy-2-methyl-propoxy)-phenoxy]-2-methyl-propionic acid ethyl ester is reacted with (2-hydroxy-5-trifluoromethyl-phenyl)-phenyl-methanone as in Example 1 to afford 1.05 g (74%) of 2-{4-[3-(2-benzoyl-4-trifluoromethyl-phenoxy)-2-methyl-propoxy]-phenoxy}-2-methyl-propionic acid. ¹H NMR (400 MHz, CDCl₃); MS (ES⁻) m/z mass calcd for C₂₈H₂₇O₆F₃ 532, found 531 (M−1).

### EXAMPLE 278

### 2-Methyl-2-{4-[2-methyl-3-(2-phenoxy-5-trifluoromethyl-phenoxy)-propoxy]-phenoxy}-propionic acid

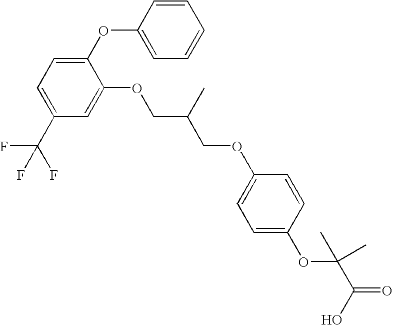

2-[4-(3-Methanesulfonyloxy-2-methyl-propoxy)-phenoxy]-2-methyl-propionic acid ethyl ester is reacted with 2-phenoxy-5-trifluoromethyl-phenol as in Example 1 to afford 0.46 g (93%) of 2-methyl-2-{4-[2-methyl-3-(2-phenoxy-5-trifluoromethyl-phenoxy)-propoxy]-phenoxy}-propionic acid. ¹H NMR (400 MHz, CDCl₃); HRMS (ES⁺) m/z mass calcd for C₂₇H₂₇O₆F₃ 505.1838, found 505.1856 (M+1).

### EXAMPLE 279

### 2-Methyl-2-{4-[2-methyl-3-(2-phenoxy-3-trifluoromethyl-phenoxy)-propoxy]-phenoxy}-propionic acid

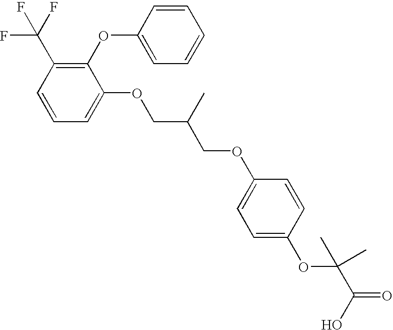

2-[4-(3-Methanesulfonyloxy-2-methyl-propoxy)-phenoxy]-2-methyl-propionic acid ethyl ester is reacted with 2-phenoxy-3-trifluoromethyl-phenol as in Example 11 to afford 0.229 g (77%) of 2-methyl-2-{4-[2-methyl-3-(2-phenoxy-3-trifluoromethyl-phenoxy)-propoxy]-phenoxy}-propionic acid. ¹H NMR (400 MHz, CDCl₃); MS (ES⁻) m/z mass calcd for C₂₇H₂₇O₆F₃ 504, found 503 (M−1).

### EXAMPLE 280

### (R)-3-{4-[3-(4-Chloro-2-phenoxy-phenoxy)-1-methyl-propylsulfanyl]-2-methyl-phenyl}-propionic acid

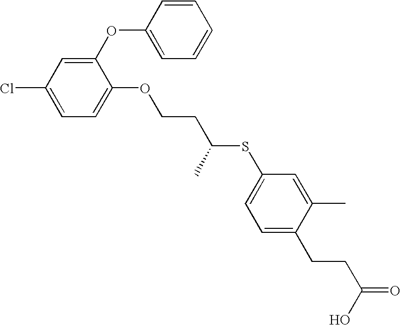

(S)-Methanesulfonic acid 3-(4-chloro-2-phenoxy-phenoxy)-1-methyl-propyl ester is reacted with 3-(4-mercapto-2-methyl-phenyl)-propionic acid methyl ester as in Example 1 to afford 0.130 g (42%) of 3-{4-[3-(4-chloro-2-phenoxy-phenoxy)-1-methyl-propylsulfanyl]-2-methyl-phenyl}-propionic acid. ¹H NMR (400 MHz, CDCl₃); HRMS (ES⁺) m/z mass calcd for C₂₆H₂₇O₄SCl 471.1397, found 471.1391 (M+1).

### EXAMPLE 281

### (R)-{4-[3-(4-Chloro-2-phenoxy-phenoxy)-1-methyl-propylsulfanyl]-2-methyl-phenoxy}-acetic acid

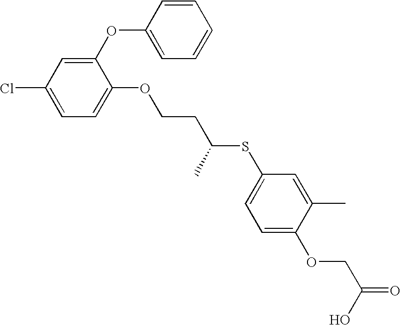

(S)-Methanesulfonic acid 3-(4-chloro-2-phenoxy-phenoxy)-1-methyl-propyl ester is reacted with (4-mercapto-2-methyl-phenoxy)-acetic acid ethyl ester as in Example 1 to afford 0.156 g (45%) of {4-[3-(4-chloro-2-phenoxy-phenoxy)-1-methyl-propylsulfanyl]-2-methyl-phenoxy}-acetic acid. ¹H NMR (400 MHz, CDCl₃); HRMS (ES⁺) m/z mass calcd for C₂₅H₂₅O₅SCl 473.1189, found 473.1190 (M+1).

### EXAMPLE 282

### (R)-3-{4-[3-(4-Chloro-2-phenoxy-phenoxy)-1-methyl-propoxy]-2-methyl-phenyl}-propionic acid

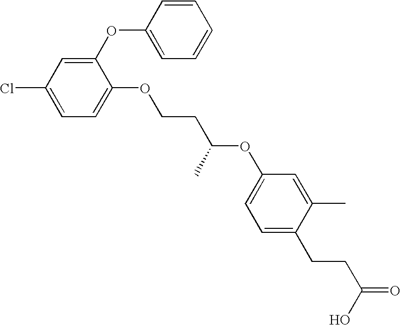

(S)-Methanesulfonic acid 3-(4-chloro-2-phenoxy-phenoxy)-1-methyl-propyl ester is reacted with 3-(4-hydroxy-2-methyl-phenyl)-propionic acid methyl ester as in Example 1 to afford 0.114 g (42%) of 3-{4-[3-(4-chloro-2-phenoxy-phenoxy)-1-methyl-propoxy]-2-methyl-phenyl}-propionic acid. ¹H NMR (400 MHz, CDCl₃); HRMS (ES⁺) m/z mass calcd for C₂₆H₂₇O₅Cl 472.1891, found 472.1894 (M+NH₄).

### EXAMPLE 283

### (R)-{4-[3-(4-Chloro-2-phenoxy-phenoxy)-1-methyl-propoxy]-2-methyl-phenylsulfanyl}-acetic acid

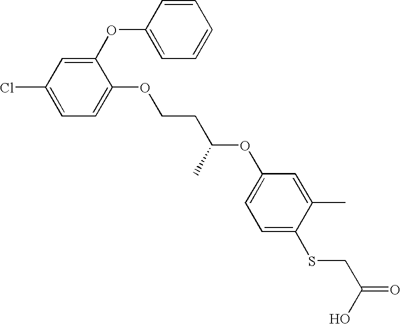

(S)-Methanesulfonic acid 3-(4-chloro-2-phenoxy-phenoxy)-1-methyl-propyl ester is reacted with (4-hydroxy-2-methyl-phenylsulfanyl)-acetic acid ethyl ester as in to afford 0.092 g (36%) of {4-[3-(4-chloro-2-phenoxy-phenoxy)-1-methyl-propoxy]-2-methyl-phenylsulfanyl}-acetic acid. ¹H NMR (400 MHz, CDCl₃); HRMS (ES⁺) m/z mass calcd for C₂₅H₂₅O₅SCl 490.1455, found 490.1440 (M+NH₄).

### EXAMPLE 284

### (S)-3-{2-Ethyl-4-[3-(2-phenoxy-4-trifluoromethyl-phenoxy)-butylsulfanyl]-phenyl}-propionic acid

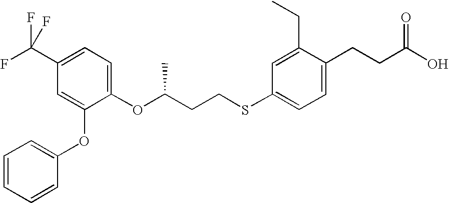

### Step A

### (R)-3-[2-Ethyl-4-(3-hydroxy-butylsulfanyl)-phenyl]-propionic acid ethyl ester

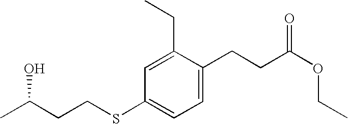

A solution of (R)-toluene-4-sulfonic acid 3-hydroxy-butyl ester (1.1 g, 4.6 mmol) and 3-(2-Ethyl-4-mercapto-phenyl)-propionic acid ethyl ester (1.0 g, 4.2 mmol) is combined in DMF (20 mL) and purged with nitrogen. The solution is treated with potassium carbonate (0.87 g, 6.3 mmol) and stirred overnight at room temperature. The reaction is then quenched with 1.0N aqueous HCl to pH=6 and extracted with diethyl ether. The organic is washed with brine, dried over sodium sulfate, filtered, and the solvent removed. The crude is purified by silica gel column chromatography using 9:1 hexanes:ethyl acetate to elute the pure product. The solvent is removed to afford 1.13 g (87%) of product. ¹H NMR (400 MHz, CDCl₃); MS (ES⁺) m/z mass calcd for C₁₇H₂₆O₃S 310, found 311 (M+1, 100%).

### Step B

### (R)-3-[2-Ethyl-4-(3-methanesulfonyloxy-butylsulfanyl)-phenyl]-propionic acid ethyl ester

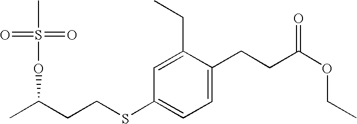

(R)-3-[2-Ethyl-4-(3-hydroxy-butylsulfanyl)-phenyl]-propionic acid ethyl ester (1.13 g, 3.6 mmol) in dichloromethane (20 mL) is cooled to 0° C. in an ice bath. The reaction is then treated with triethylamine (0.44 g, 4.4 mmol) and methanesulfonyl chloride (0.46 g, 4.0 mmol). The reaction is allowed to warm to room temperature and stirred for 2 hr. The reaction is then quenched with 1.0N aqueous HCl to pH=6, and the aqueous is extracted with dichloromethane. The organic is washed with brine, dried over sodium sulfate, and filtered. The solvent is removed to afford 1.28 g (91%) of product. ¹H NMR (400 MHz, CDCl₃); MS (ES⁺) m/z mass calcd for C₁₈H₂₈O₅S₂ 388, found 406 (M+NH₄, 100%).

### Step C

### (S)-3-{2-Ethyl-4-[3-(2-phenoxy-4-trifluoromethyl-phenoxy)-butylsulfanyl]-phenyl}-propionic acid

A solution of (R)-3-[2-ethyl-4-(3-methanesulfonyloxy-butylsulfanyl)-phenyl]-propionic acid ethyl ester (0.15 g, 0.39 mmol) and 2-Phenoxy-4-trifluoromethyl-phenol (0.098 g, 0.39 mmol) in DMF (3 mL) is treated with cesium carbonate (0.151 g, 0.46 mmol). The reaction is heated to 60° C. and stirred overnight. The reaction is then treated with 5N aqueous sodium hydroxide (0.4 mL) and stirred for an additional 2 hr. The reaction is cooled and quenched with 1N aqueous hydrochloric acid to pH=4. The aqueous solution is extracted with diethyl ether. The organic is washed with brine and dried over sodium sulfate. The organic is filtered, and the solvent is removed to afford the crude product. The crude is purified by reverse phase HPLC. The solvent is removed to afford 0.081 g (40%) of the desired product. ¹H NMR (400 MHz, CDCl₃); MS (ES⁺) m/z mass calcd for C₂₈H₂₉F₃O₄S 518, found 536 (M+NH₄, 100%); MS (ES⁻) found 517 (M−1, 100%).

### EXAMPLE 285

### (S)-{3-[3-(2-Phenoxy-4-trifluoromethyl-phenoxy)-butylsulfanyl]-phenyl}-acetic acid

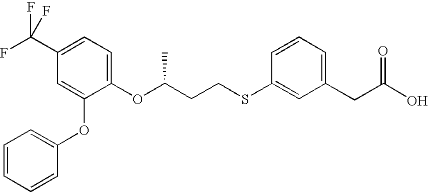

### Step A

### (R)-[3-(3-Hydroxy-butylsulfanyl)-phenyl]-acetic acid methyl ester

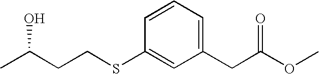

The procedure from Example 284, Step A is utilized with (3-mercapto-phenyl)-acetic acid methyl ester. The reaction affords 1.1 g (79%) of product. ¹H NMR (400 MHz, CDCl₃); MS (ES⁺) m/z mass calcd for C₁₇H₂₆O₃S 254, found 255 (M+1, 100%).

### Step B

### (R)-[3-(3-Methanesulfonyloxy-butylsulfanyl)-phenyl]-acetic acid methyl ester

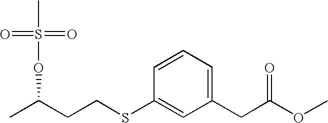

The procedure from Example 284, Step B is utilized with (R)-[3-(3-hydroxy-butylsulfanyl)-phenyl]-acetic acid methyl ester. The reaction affords 1.37 g (95%) of product. ¹H NMR (400 MHz, CDCl₃); MS (ES⁺) m/z mass calcd for C₁₄H₂₀O₅S₂ 332, found 350 (M+NH₄, 100%).

### Step C

### (S)-{3-[3-(2-Phenoxy-4-trifluoromethyl-phenoxy)-butylsulfanyl]-phenyl}-acetic acid

The procedure from Example 284, Step C is utilized with (R)-[3-(3-methanesulfonyloxy-butylsulfanyl)-phenyl]-acetic acid methyl ester. The reaction affords 0.053 g (37%) of product. ¹H NMR (400 MHz, CDCl₃); MS (ES⁺) m/z mass calcd for C₂₅H₂₃F₃O₄S 476, found 494 (M+NH₄, 100%); MS (ES⁻) found 475 (M−1, 100%).

### EXAMPLE 286

### (S)-{3-[3-(4-Chloro-2-phenoxy-phenoxy)-butylsulfanyl]-phenyl}-acetic acid

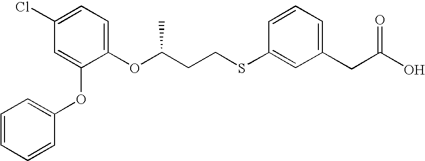

The procedure from Example 284, Step C is utilized with (R)-[3-(3-methanesulfonyloxy-butylsulfanyl)-phenyl]-acetic acid methyl ester and 4-chloro-2-phenoxy-phenol. The reaction affords 0.053 g (40%) of product. ¹H NMR (400 MHz, CDCl₃); MS (ES⁺) m/z mass calcd for C₂₄H₂₃ClO₄S 442, found 460 (M+NH₄, 100%); MS (ES⁻) found 441 (M−1, 100%).

### EXAMPLE 287

### (S)-{3-[3-(2-Benzoyl-4-ethyl-phenoxy)-butylsulfanyl]-phenyl}-acetic acid

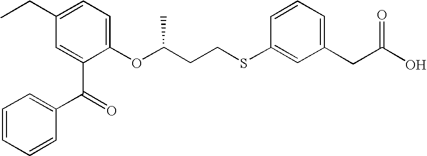

The procedure from Example 284, Step C is utilized with (R)-[3-(3-methanesulfonyloxy-butylsulfanyl)-phenyl]-acetic acid methyl ester and (5-ethyl-2-hydroxy-phenyl)-phenyl-methanone. The reaction affords 0.051 g (40%) of product. ¹H NMR (400 MHz, CDCl₃); MS (ES⁺) m/z mass calcd for C₂₇H₂₈O₄S 448, found 449 (M+1, 100%); MS (ES⁻) found 447 (M−1, 100%).

### EXAMPLE 288

### (S)-{3-[3-(4-Chloro-2-phenoxy-phenoxy)-butoxy]-phenyl}-acetic acid

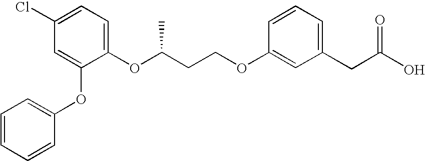

The procedure from Example 284, Step C is utilized with (S)-methanesulfonic acid 3-(4-chloro-2-phenoxy-phenoxy)-butyl ester and (3-hydroxy-phenyl)-acetic acid methyl ester. The reaction affords 0.1 g (58%) of product. ¹H NMR (400 MHz, CDCl₃); MS (ES⁺) m/z mass calcd for C₂₄H₂₃ClO₅ 426, found 444 (M+NH₄, 100%); MS (ES⁻) found 425 (M−1,100%).

### EXAMPLE 289

### (S)-3-{3-[3-(4-Chloro-2-phenoxy-phenoxy)-butoxy]-phenyl}-propionic acid

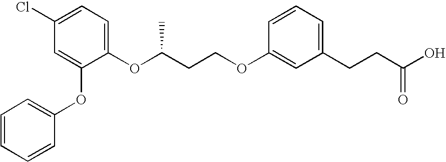

The procedure from Example 284, Step C is utilized with (S)-methanesulfonic acid 3-(4-chloro-2-phenoxy-phenoxy)-butyl ester and 3-(3-hydroxy-phenyl)-propionic acid methyl ester. The reaction affords 0.12 g (67%) of product. ¹H NMR (400 MHz, CDCl₃); MS (ES⁺) m/z mass calcd for C₂₅H₂₅ClO₅ 440, found 458 (M+NH₄, 100%); MS (ES⁻) found 439 (M−1, 100%).

### EXAMPLE 290

### (S)-3-{2-Ethyl-4-[3-(2-phenoxy-4-trifluoromethoxy-phenoxy)-butylsulfanyl]-phenyl}-propionic acid

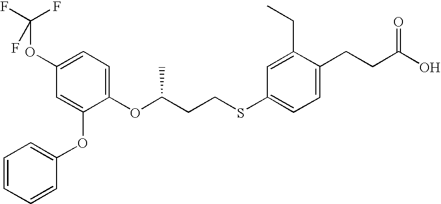

### Step A

### (S)-3-{4-[3-(2-Bromo-4-trifluoromethoxy-phenoxy)-butylsulfanyl]-2-ethyl-phenyl}-propionic acid ethyl ester

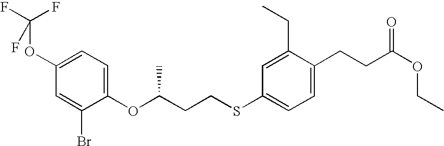

A solution of (R)-3-[2-ethyl-4-(3-methanesulfonyloxy-butylsulfanyl)-phenyl]-propionic acid ethyl ester (0.15 g, 0.39 mmol) and 2-Bromo-4-trifluoromethoxy-phenol (0.099 g, 0.39 mmol) in DMF (3 mL) is treated with cesium carbonate (0.151 g, 0.46 mmol). The reaction is heated to 60° C. and stirred overnight. The reaction is cooled and quenched with 1N aqueous hydrochloric acid to pH=6. The aqueous solution is extracted with diethyl ether. The organic is washed with brine and dried over sodium sulfate. The organic is filtered, and the solvent is removed to afford the crude product. The crude is purified by silica gel column chromatography using 4:1 hexanes:ethylacetate to elute the pure product. The solvent is removed to afford 0.185 g (87%) of product. ¹H NMR (400 MHz, CDCl₃); MS (ES⁺) m/z mass calcd for C₂₄H₂₈BrF₃O₄ 548, found 566 (M+NH₄, 100%).

### Step B

### (S)-3-{2-Ethyl-4-[3-(2-phenoxy-4-trifluoromethoxy-phenoxy)-butylsulfanyl]-phenyl}-propionic acid

A solution of (S)-3-{4-[3-(2-bromo-4-trifluoromethoxy-phenoxy)-butylsulfanyl]-2-ethyl-phenyl}-propionic acid ethyl ester (0.185 g, 0.34 mmol), phenol (0.095 g, 1.01 mmol), copper(I) chloride (17 mg, 0.17 mmol), cesium carbonate (0.329 g, 1.01 mmol) and 2,2,6,6-tetramethyl-3,5-heptanedione (0.02 mL, 0.08 mmol) in NMP (5 mL) is purged with nitrogen. The reaction is heated to 120° C. and stirred overnight. The reaction is cooled to 60° C. and treated with 5N aqueous NaOH. The reaction is stirred an additional 2 hr, and then cooled and quenched with 1N aqueous hydrochloric acid to pH=4. The aqueous solution is extracted with diethyl ether. The organic is washed with brine and dried over sodium sulfate. The organic is filtered, and the solvent is removed to afford the crude product. The crude is purified by reverse phase HPLC to afford 0.107 g (59%) of product. ¹H NMR (400 MHz, CDCl₃); MS (ES⁺) m/z mass calcd for C₂₈H₂₉F₃O₅S 534, found 532 (M+NH₄, 100%); MS (ES−) found 533 (M−1, 100%).

### EXAMPLE 291

### (S)-3-{4-[3-(5-Chloro-2′-fluoro-biphenyl-2-yloxy)-butoxy]-2-methyl-phenyl}-propionic acid

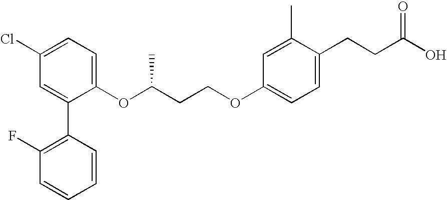

### Step A

### (S)-3-(2-Bromo-4-chloro-phenoxy)-butan-1-ol

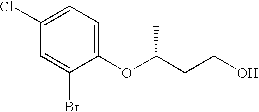

A solution of (R)-acetic acid 3-(toluene-4-sulfonyloxy)-butyl ester (4.0 g, 14 mmol), 2-bromo-4-chlorophenol (2.9 g, 14 mmol), and cesium carbonate (5.45 g, 16.8 mmol) is combined in DMF (100 mL). The solution is heated to 60° C. and stirred overnight. The reaction is cooled and quenched with 1N aqueous HCl to pH=6. The aqueous is extracted with diethyl ether. The organic is washed with brine, dried over sodium sulfate, filtered, and the solvent is removed. The residue is taken up in MeOH (50 mL) and treated with potassium carbonate (5.8 g, 42 mmol). The reaction is stirred at room temperature for 3 hr. The reaction is filtered and the filtrate is concentrated. The crude is purified by silica gel column chromatography using 4:1 hexanes:ethylacetate to elute the pure product. The solvent is removed to afford 3.29 g (84%) of product. ¹H NMR (400 MHz, CDCl₃); MS (ES⁺) m/z mass calcd for C₁₀H₁₂BrClO₂ 278, found 301 (M+Na, 100%).

### Step B

### (S)-Methanesulfonic acid 3-(2-bromo-4-chloro-phenoxy)-butyl ester

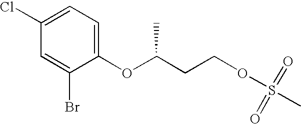

The procedure from Example 284, Step B is utilized with (S)-3-(2-bromo-4-chloro-phenoxy)-butan-1-ol. The reaction affords 4.14 g (99%) of product. ¹H NMR (400 MHz, CDCl₃); MS (ES⁺) m/z mass calcd for C₁₁H₁₄BrClO₄S 356, found 374 (M+NH₄, 100%).

### Step C

### (S)-3-{4-[3-(2-Bromo-4-chloro-phenoxy)-butoxy]-2-methyl-phenyl}-propionic acid methyl ester

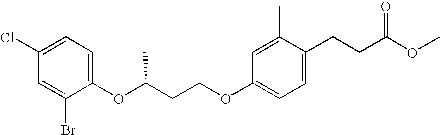

The procedure from Example 290, Step A is utilized with (S)-methanesulfonic acid 3-(2-bromo-4-chloro-phenoxy)-butyl ester and 3-(4-hydroxy-2-methyl-phenyl)-propionic acid methyl ester. The reaction affords 3.59 g (68%) of product. ¹H NMR (400 MHz, CDCl₃); MS (ES⁺) m/z mass calcd for C₂₁H₂₄BrClO₄ 454, found 472 (M+NH₄, 100%); MS (ES⁻) found 471 (M−1,100%).

### Step D

### (S)-3-{4-[3-(5-Chloro-2′-fluoro-biphenyl-2-yloxy)-butoxy]-2-methyl-phenyl}-propionic acid methyl ester

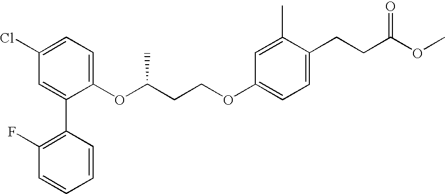

A solution of (S)-3-{4-[3-(2-bromo-4-chloro-phenoxy)-butoxy]-2-methyl-phenyl}-propionic acid methyl ester (0.1 g, 0.22 mmol), 2-fluorophenylboronic acid (0.092 g, 0.66 mmol), cesium fluoride (0.117 g, 0.77 mmol), and 1,1′-bis(diphenylphosphino)ferrocene palladium(II)chloride complex with dichloromethane (0.032 g, 0.04 mmol) in acetonitrile (10 mL) is purged with nitrogen. The reaction is heated to reflux and stirred for 3 hr. The reaction is quenched with 1N aqueous hydrochloric acid to pH=6. The aqueous is extracted with diethyl ether. The organic is washed with brine, dried over sodium sulfate, filtered, and solvent is removed. The crude is purified using silica gel column chromatography with 9:1 hexanes:ethyl acetate to elute the pure product. The solvent is removed to afford 0.026 g (25%). ¹H NMR (400 MHz, CDCl₃); MS (ES⁺) m/z mass calcd for C₂₇H₂₈ClFO₄ 470, found 488 (M+NH₄, 100%).

### Step E

### (S)-3-{4-[3-(5-Chloro-2′-fluoro-biphenyl-2-yloxy)-butoxy]-2-methyl-phenyl}-propionic acid

A solution of (S)-3-{4-[3-(5-chloro-2′-fluoro-biphenyl-2-yloxy)-butoxy]-2-methyl-phenyl}-propionic acid methyl ester (0.026 g, 0.05 mmol) in methanol (5 mL) is treated with 5N aqueous sodium hydroxide (0.1 mL). The reaction is heated to reflux and stirred for 3 hr. The reaction is cooled to room temperature and the pH is adjusted to pH=4 with 1N aqueous hydrochloric acid. The aqueous is extracted with diethyl ether. The organic is washed with brine, dried over sodium sulfate, and filtered. The solvent is removed to afford 0.023 g (92%) of product. ¹H NMR (400 MHz, CDCl₃); MS (ES⁺) m/z mass calcd for C₂₆H₂₆ClFO₄ 456, found 474 (M+NH₄, 100%); MS (ES−) found 455 (M−1, 100%).

### EXAMPLE 292

### (S)-3-{4-[3-(5-Chloro-2′-methoxy-biphenyl-2-yloxy)-butoxy]-2-methyl-phenyl}-propionic acid

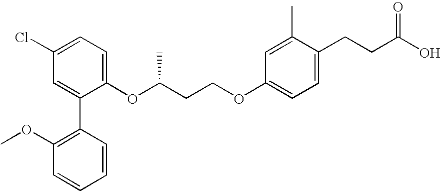

### Step A

### (S)-3-{4-[3-(5-Chloro-2′-methoxy-biphenyl-2-yloxy)-butoxy]-2-methyl-phenyl}-propionic acid methyl ester

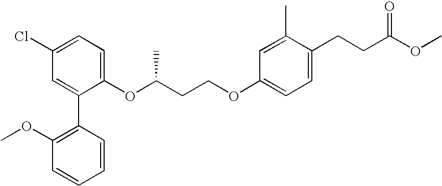

The procedure from Example 291, Step D is utilized with 2-methoxyphenyl boronic acid. The reaction affords 0.076 g (75%) of product. ¹H NMR (400 MHz, CDCl₃); MS (ES⁺) m/z mass calcd for C₂₈H₃₁ClO₅ 482, found 500 (M+NH₄, 100%).

### Step B

### (S)-3-{4-[3-(5-Chloro-2′-methoxy-biphenyl-2-yloxy)-butoxy]-2-methyl-phenyl}-propionic acid

The procedure from Example 291, Step E is utilized with (S)-3-{4-[3-(5-chloro-2′-methoxy-biphenyl-2-yloxy)-butoxy]-2-methyl-phenyl}-propionic acid methyl ester. The reaction affords 0.063 g (85%) of product. ¹H NMR (400 MHz, CDCl₃); MS (ES⁺) m/z mass calcd for C₂₇H₂₉ClO₅ 468, found 486 (M+NH₄, 100%); MS (ES⁻) found 467 (M−1, 100%).

### EXAMPLE 293

### (S)-3-(4-{3-[4-Chloro-2-(2-fluoro-phenoxy)-phenoxy]-butoxy}-2-methyl-phenyl)-propionic acid

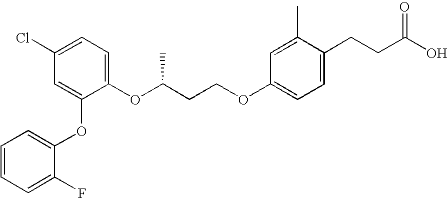

### Step A

### 4-Chloro-2-(2-fluoro-phenoxy)-benzaldehyde

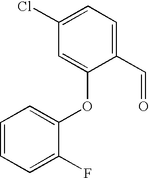

A solution of 4-chloro-2-fluorobenzaldehyde (1.0 g, 6.3 mmol) and 2-fluorophenol (0.78 g, 6.9 mmol) in DMSO (10 mL) is treated with potassium carbonate (1.04 g, 7.6 mmol). The reaction is heated to 100° C. and stirred overnight. The reaction is cooled to room temperature and quenched with 1N aqueous hydrochloric acid to pH=6. The aqueous is extracted with diethyl ether. The organic is washed with brine, dried over sodium sulfate, filtered, and the solvent is removed. The crude is purified by silica gel column chromatography using 4:1 hexanes:acetone to elute the pure product. The solvent is removed to afford 0.8 g (51%) of product. ¹H NMR (400 MHz, CDCl₃), TLC (1:1 Hexanes:EtOAc) R_{f}=0.8.

### Step B

### 4-Chloro-2-(2-fluoro-phenoxy)-phenol

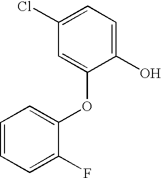

A solution of 4-chloro-2-(2-fluoro-phenoxy)-benzaldehyde (0.8 g, 3.2 mmol) in chloroform (10 mL) is treated with m-CPBA (2.75 g, 16 mmol). The reaction is heated to reflux and stirred for 2 hr. The reaction is cooled to room temperature and quenched with 10% aqueous NaHSO₄. The aqueous is extracted with diethyl ether. The organic is washed with brine, dried over sodium sulfate, and the solvent is removed. The crude is diluted in methanol (20 mL) and treated with potassium carbonate (1.32 g, 9.6 mmol). The reaction is stirred for 30 minutes at room temperature. The reaction is filtered and the solvent removed. The crude is purified by silica gel column chromatography using 4:1 hexanes:acetone to elute the pure product. The solvent is removed to afford 0.64 g (84%) of product. ¹H NMR (400 MHz, CDCl₃). MS (ES⁻) m/z mass calcd for C₁₂H₈ClFO₂ 238, found 237 (M−1, 100%).

### Step C

### (S)-3-(4-{3-[4-Chloro-2-(2-fluoro-phenoxy)-phenoxy]-butoxy}-2-methyl-phenyl)-propionic acid methyl ester

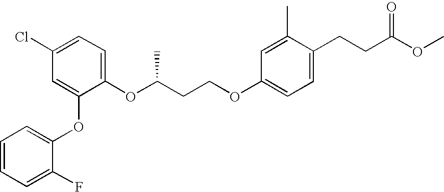

The procedure from Example 290, Step A is utilized with 4-chloro-2-(2-fluoro-phenoxy)-phenol and 3-[4-(3-Methanesulfonyloxy-butoxy)-2-methyl-phenyl]-propionic acid methyl ester. The reaction affords 0.195 g (92%) of product. ¹H NMR (400 MHz, CDCl₃). MS (ES⁺) m/z mass calcd for C₂₇H₂₈ClFO₅ 486, found 504 (M+NH₄, 100%).

### Step D

### (S)-3-(4-{3-[4-Chloro-2-(2-fluoro-phenoxy)-phenoxy]-butoxy}-2-methyl-phenyl)-propionic acid

The procedure from Example 291, Step E is utilized with (S)-3-(4-{3-[4-chloro-2-(2-fluoro-phenoxy)-phenoxy]-butoxy}-2-methyl-phenyl)-propionic acid methyl ester. The reaction affords 0.166 g (88%) of product. ¹H NMR (400 MHz, CDCl₃). MS (ES⁺) m/z mass calcd for C₂₆H₂₆ClFO₅ 472, found 490 (M+NH₄, 100%); MS (ES⁻) found 471.

### EXAMPLE 294

### (S)-3-(4-{3-[4-Chloro-2-(2-fluoro-phenoxy)-phenoxy]-butoxy}-2-ethyl-phenyl)-propionic acid

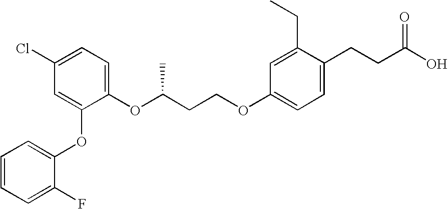

### Step A

### (S)-3-(4-{3-[4-Chloro-2-(2-fluoro-phenoxy)-phenoxy]-butoxy}-2-ethyl-phenyl)-propionic acid ethyl ester

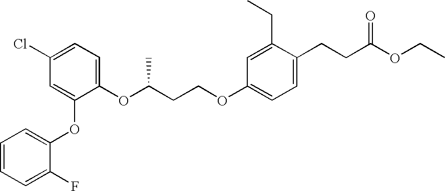

The procedure from Example 290, Step A is utilized with 4-chloro-2-(2-fluoro-phenoxy)-phenol and 3-(2-ethyl-4-hydroxy-phenyl)-propionic acid ethyl ester. The reaction affords 0.135 g (65%) of product. ¹H NMR (400 MHz, CDCl₃). MS (ES⁺) m/z mass calcd for C₂₉H₃₂ClFO₅ 514, found 532 (M+NH₄, 100%).

### Step B

### (S)-3-(4-{3-[4-Chloro-2-(2-fluoro-phenoxy)-phenoxy]-butoxy}-2-ethyl-phenyl)-propionic acid

A solution of (S)-3-(4-{3-[4-chloro-2-(2-fluoro-phenoxy)-phenoxy]-butoxy}-2-ethyl-phenyl)-propionic acid ethyl ester (0.135 g, 0.26 mmol) in ethanol (10 mL) is treated with 5N aqueous sodium hydroxide (0.5 mL, 2.6 mmol). The reaction is heated to reflux and stirred for 3 hr. The reaction is cooled to room temperature and quenched with 1N aqueous hydrochloric acid to pH=4. The aqueous is extracted with diethyl ether. The organic is washed with brine, dried over sodium sulfate, and filtered. The solvent is removed to afford 0.108 g (85%) of product. ¹H NMR (400 MHz, CDCl₃). MS (ES⁺) m/z mass calcd for C₂₇H₂₈ClFO₅ 486, found 504 (M+NH₄, 100%); MS (ES⁻) found 485.

### EXAMPLE 295

### (S)-3-{2-Methyl-4-[3-(2-phenoxy-4-trifluoromethoxy-phenoxy)-butylsulfanyl]-phenyl}-propionic acid

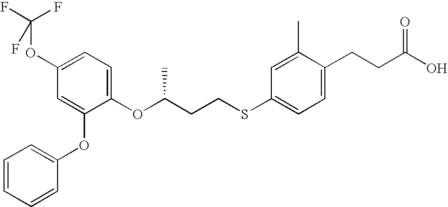

### Step A

### (S)-3-{4-[3-(2-Bromo-4-trifluoromethoxy-phenoxy)-butylsulfanyl]-2-methyl-phenyl}-propionic acid methyl ester

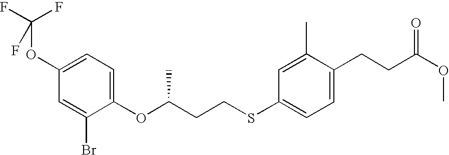

The procedure from Example 290, Step A is utilized with (R)-3-[4-(3-methanesulfonyloxy-butylsulfanyl)-2-methyl-phenyl]-propionic acid methyl ester to afford 0.13 g (90%) of product. ¹H NMR (400 MHz, CDCl₃); MS (ES⁺) m/z mass calcd for C₂₂H₂₄BrF₃O₄S 520, found 538 (M+NH₄, 100%).

### Step B

### (S)-3-{2-Methyl-4-[3-(2-phenoxy-4-trifluoromethoxy-phenoxy)-butylsulfanyl]-phenyl}-propionic acid

The procedure from Example 290, Step B is utilized with (S)-3-{4-[3-(2-bromo-4-trifluoromethoxy-phenoxy)-butylsulfanyl]-2-methyl-phenyl}-propionic acid methyl ester to afford 0.009 g (9%) of product. ¹H NMR (400 MHz, CDCl₃); MS (ES⁺) m/z mass calcd for C₂₇H₂₇F₃O₅S 520, found 538 (M+NH₄, 100%); MS (ES⁻) found 519 (M−1, 100%).

### EXAMPLE 296

### (S)-{2-Methyl-4-[3-(2-phenoxy-4-trifluoromethyl-phenoxy)-butylsulfanyl]-phenoxy}-acetic acid

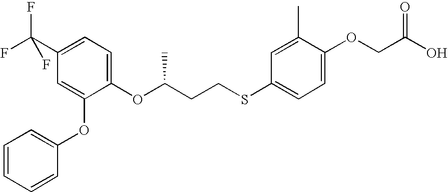

### Step A

### (S)-3-(2-Phenoxy-4-trifluoromethyl-phenoxy)-butan-1-ol

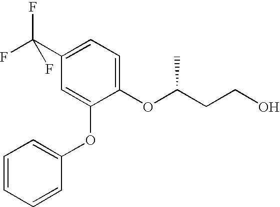

The procedure from Example 291, Step A is utilized 2-phenoxy-4-trifluoromethyl-phenol to afford 3.29 g (85%) of product. ¹H NMR (400 MHz, CDCl₃); MS (ES⁺) m/z mass calcd for C₁₇H₁₇F₃O₃ 326, found 327 (M+1, 60%); 344 (M+NH₄, 100%); MS (ES⁻) found 385 (M+CH₃COO⁻, 100%).

### Step B

### (S)-Methanesulfonic acid 3-(2-phenoxy-4-trifluoromethyl-phenoxy)-butyl ester

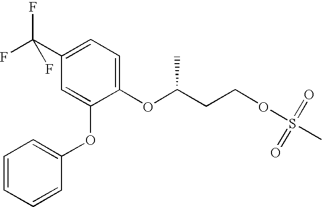

The procedure from Example 284, Step B is utilized with (S)-3-(2-phenoxy-4-trifluoromethyl-phenoxy)-butan-1-ol afford 3.8 g (95%) of product. ¹H NMR (400 MHz, CDCl₃); MS (ES⁺) m/z mass calcd for C₁₈H₁₉F₃O₅S 404, found 422 (M+NH₄, 100%).

### Step C

### (S)-{2-Methyl-4-[3-(2-phenoxy-4-trifluoromethyl-phenoxy)-butylsulfanyl]-phenoxy}-acetic acid ethyl ester

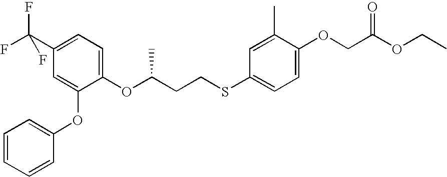

The procedure from Example 290, Step A is utilized with (S)-methanesulfonic acid 3-(2-phenoxy-4-trifluoromethyl-phenoxy)-butyl ester and (4-Mercapto-2-methyl-phenoxy)-acetic acid ethyl ester to afford 0.082 g (62%) of product. ¹H NMR (400 MHz, CDCl₃); MS (ES⁺) m/z mass calcd for C₂₈H₂₉F₃O₅S 534, found 535 (M+1, 20%), found 552 (M+NH₄, 100%); MS (ES⁻) found 593 (+CH3COO, 100%).

### Step D

### (S)-{2-Methyl-4-[3-(2-phenoxy-4-trifluoromethyl-phenoxy)-butylsulfanyl]-phenoxy}-acetic acid

The procedure from Example 294, Step B is utilized with (S)-{2-methyl-4-[3-(2-phenoxy-4-trifluoromethyl-phenoxy)-butylsulfanyl]-phenoxy}-acetic acid ethyl ester to afford 0.072 g (92%) of product. ¹H NMR (400 MHz, CDCl₃); MS (ES⁺) m/z mass calcd for C₂₆H₂₅F₃O₅S 506, found 524 (M+NH₄, 100%); MS (ES⁻) found 505 (M−1, 100%).

### EXAMPLE 297

### (S)-{2-Ethyl-4-[3-(2-phenoxy-4-trifluoromethyl-phenoxy)-butylsulfanyl]-phenoxy}-acetic acid

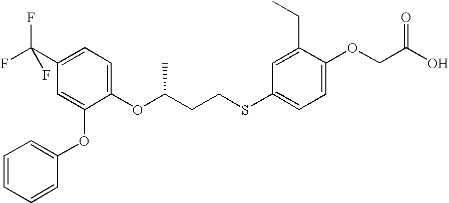

### Step A

### (S)-{2-Ethyl-4-[3-(2-phenoxy-4-trifluoromethyl-phenoxy)-butylsulfanyl]-phenoxy}-acetic acid ethyl ester

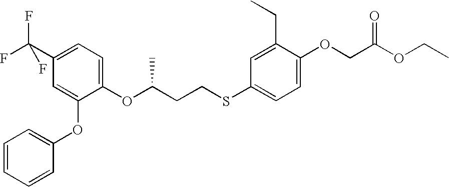

The procedure from Example 290, Step A is utilized with (S)-methanesulfonic acid 3-(2-phenoxy-4-trifluoromethyl-phenoxy)-butyl ester and (4-mercapto-2-ethyl-phenoxy)-acetic acid ethyl ester to afford 0.115 g (85%) of product. ¹H NMR (400 MHz, CDCl₃); MS (ES⁺) m/z mass calcd for C₂₉H₃₁F₃O₅S 548, found 549 (M+1,20%), found 566 (+NH₄, 100%); MS (ES⁻) found 607 (M+CH₃COO⁻, 100%).

### Step B

### (S)-{2-Ethyl-4-[3-(2-phenoxy-4-trifluoromethyl-phenoxy)-butylsulfanyl]-phenoxy}-acetic acid

The procedure from Example 294, Step B is utilized with (S)-{2-ethyl-4-[3-(2-phenoxy-4-trifluoromethyl-phenoxy)-butylsulfanyl]-phenoxy}-acetic acid ethyl ester to afford 0.072 g (92%) of product. ¹H NMR (400 MHz, CDCl₃); MS (ES⁺) m/z mass calcd for C₂₇H₂₇F₃O₅S 520, found 538 (M+NH₄, 100%); MS (ES⁻) found 519 (M−1, 100%).

### EXAMPLE 298

### (S)-3-(4-{3-[4-Chloro-2-(3-fluoro-phenoxy)-phenoxy]-butoxy}-2-methyl-phenyl)-propionic acid

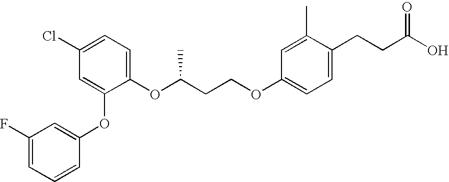

### Step A

### 4-Chloro-2-(3-fluoro-phenoxy)-benzaldehyde

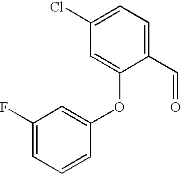

The procedure from Example 293, Step A is utilized with 3-fluorophenol to afford 1.4 g (89%) of product. ¹H NMR (400 MHz, CDCl₃), TLC (1:1 hexanes:EtOAc) R_{f}=0.8.

### Step B

### 4-Chloro-2-(3-fluoro-phenoxy)-phenol

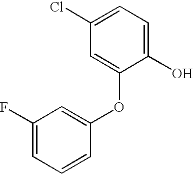

The procedure from Example 293, Step B is utilized with 4-chloro-2-(3-fluoro-phenoxy)-benzaldehyde to afford 0.914 g (69%) of product. ¹H NMR (400 MHz, CDCl₃). MS (ES⁻) m/z mass calcd for C₁₂H₈ClFO₂ 238, found 237 (M−1, 100%).

### Step C

### (S)-3-(4-{3-[4-Chloro-2-(3-fluoro-phenoxy)-phenoxy]-butoxy}-2-methyl-phenyl)-propionic acid methyl ester

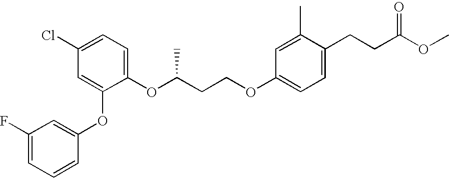

The procedure from Example 290, Step A is utilized with 4-chloro-2-(3-fluoro-phenoxy)-phenol and 3-[4-(3-methanesulfonyloxy-butoxy)-2-methyl-phenyl]-propionic acid methyl ester. The reaction affords 0.09 g (64%) of product. ¹H NMR (400 MHz, CDCl₃). MS (ES⁺) m/z mass calcd for C₂₇H₂₈ClFO₅ 486, found 504 (M+NH₄, 100%).

### Step D

### (S)-3-(4-{3-[4-Chloro-2-(3-fluoro-phenoxy)-phenoxy]-butoxy}-2-methyl-phenyl)-propionic acid

The procedure from Example 291, Step E is utilized with (S)-3-(4-{3-[4-chloro-2-(3-fluoro-phenoxy)-phenoxy]-butoxy}-2-methyl-phenyl)-propionic acid methyl ester. The reaction affords 0.06 g (91%) of product. ¹H NMR (400 MHz, CDCl₃). MS (ES⁺) m/z mass calcd for C₂₆H₂₆ClFO₅ 472, found 490 (M+NH₄, 100%); MS (ES⁻) found 471.

### EXAMPLE 299

### (S)-3-(4-{3-[4-Chloro-2-(4-fluoro-phenoxy)-phenoxy]-butoxy}-2-methyl-phenyl)-propionic acid

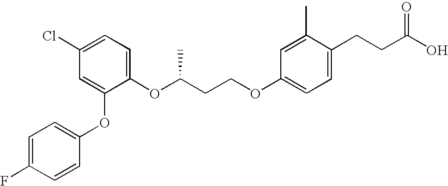

### Step A

### 4-Chloro-2-(4-fluoro-phenoxy)-benzaldehyde

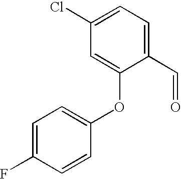

The procedure from Example 293, Step A is utilized with 3-fluorophenol to afford 1.2 g (76%) of product. ¹H NMR (400 MHz, CDCl₃), TLC (1:1 hexanes:EtOAc) R_{f}=0.8.

### Step B

### 4-Chloro-2-(4-fluoro-phenoxy)-phenol

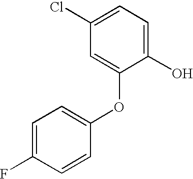

The procedure from Example 293, Step B is utilized with 4-chloro-2-(4-fluoro-phenoxy)-benzaldehyde to afford 0.994 g (87%) of product. ¹H NMR (400 MHz, CDCl₃). MS (ES⁻) m/z mass calcd for C₁₂H₈ClFO₂ 238, found 237 (M−1, 100%).

### Step C

### (S)-3-(4-{3-[4-Chloro-2-(4-fluoro-phenoxy)-phenoxy]-butoxy}-2-methyl-phenyl)-propionic acid methyl ester

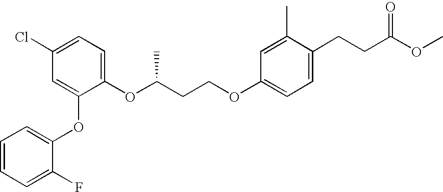

The procedure from Example 290, Step A is utilized with 4-chloro-2-(4-fluoro-phenoxy)-phenol and 3-[4-(3-Methanesulfonyloxy-butoxy)-2-methyl-phenyl]-propionic acid methyl ester. The reaction affords 0.09 g (64%) of product. ¹H NMR (400 MHz, CDCl₃). MS (ES⁺) m/z mass calcd for C₂₇H₂₈ClFO₅ 486, found 504 (M+NH₄, 100%).

### Step D

### (S)-3-(4-{3-[4-Chloro-2-(4-fluoro-phenoxy)-phenoxy]-butoxy}-2-methyl-phenyl)-propionic acid

The procedure from Example 291, Step E is utilized with (S)-3-(4-{3-[4-chloro-2-(3-fluoro-phenoxy)-phenoxy]-butoxy}-2-methyl-phenyl)-propionic acid methyl ester. The reaction affords 0.06 g (91%) of product. ¹H NMR (400 MHz, CDCl₃). MS (ES⁺) m/z mass calcd for C₂₆H₂₆ClFO₅ 472, found 490 (M+NH₄, 100%); MS (ES⁻) found 471.

### EXAMPLE 300

### (S)-{2-Ethyl-4-[3-(2-phenoxy-4-trifluoromethyl-phenoxy)-butoxy]-phenylsulfanyl}-acetic acid

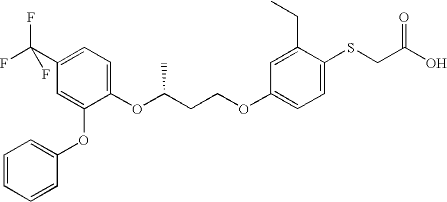

The procedure from Example 284, Step C is utilized with (S)-methanesulfonic acid 3-(2-phenoxy-4-trifluoromethyl-phenoxy)-butyl ester and (2-ethyl-4-hydroxy-phenylsulfanyl)-acetic acid ethyl ester to afford 0.068 g (53%) of product. ¹H NMR (400 MHz, CDCl₃). MS (ES⁺) m/z mass calcd for C₂₇H₂₇F₃O₅S 520, found 538 (M+NH₄, 100%); MS (ES⁻) found 519.

### EXAMPLE 301

### (S)-3-{4-[3-(2′-fluoro-5-Trifluoromethyl-biphenyl-2-yloxy)-butoxy]-2-methyl-phenyl}-propionic acid

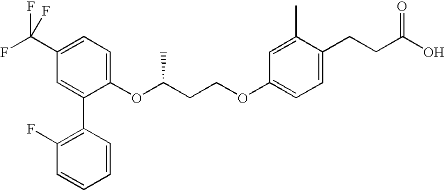

### Step A

### (S)-3-(2-Bromo-4-trifluoromethyl-phenoxy)-butan-1-ol

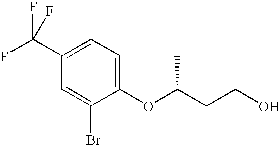

The procedure from Example 291, Step A is utilized with 2-bromo-4-trifluoromethylphenol to afford 0.45 g (51%) of product. ¹H NMR (400 MHz, CDCl₃); MS (ES⁺) m/z mass calcd for C₁₁H₁₂BrF₃O₂ 312, found 335 (M+Na, 100%).

### Step B

### (S)-Methanesulfonic acid 3-(2-bromo-4-trifluoromethyl-phenoxy)-butyl ester

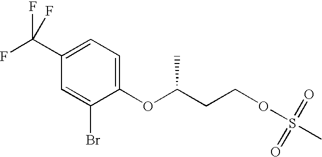

The procedure from Example 284, Step B is utilized with (S)-3-(2-bromo-4-trifluoromethyl-phenoxy)-butan-1-ol. The reaction affords 0.56 g (100%) of product. ¹H NMR (400 MHz, CDCl₃); MS (ES⁺) m/z mass calcd for C₁₂H₁₄BrClF₃O₄S 390, found 408 (M+NH₄, 100%).

### Step C

### (S)-3-{4-[3-(2-Bromo-4-trifluoromethyl-phenoxy)-butoxy]-2-methyl-phenyl}-propionic acid methyl ester

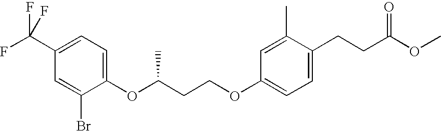

The procedure from Example 290, Step A is utilized with (S)-methanesulfonic acid 3-(2-bromo-4-trifluoromethyl-phenoxy)-butyl ester and 3-(4-hydroxy-2-methyl-phenyl)-propionic acid methyl ester. The reaction affords 0.43 g (61%) of product. ¹H NMR (400 MHz, CDCl₃); MS (ES⁺) m/z mass calcd for C₂₂H₂₄BrF₃O₄ 488, found 508 (M+NH₄, 100%).

### Step D

### (S)-3-{4-[3-(2′-Fluoro-5-trifluoromethyl-biphenyl-2-yloxy)-butoxy]-2-methyl-phenyl}-propionic acid methyl ester

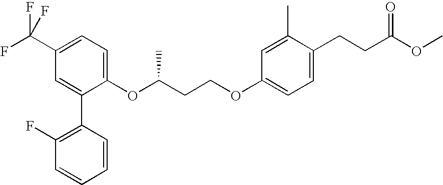

The procedure from Example 291, Step D is utilized with (S)-3-{4-[3-(2-bromo-4-trifluoromethyl-phenoxy)-butoxy]-2-methyl-phenyl}-propionic acid methyl ester to afford 0.148 g (72%). ¹H NMR (400 MHz, CDCl₃); MS (ES⁺) m/z mass calcd for C₂₈H₂₈F₄O₄ 504, found 522 (M+NH₄, 100%).

### Step E

### (S)-3-{4-[3-(2′-fluoro-5-Trifluoromethyl-biphenyl-2-yloxy)-butoxy]-2-methyl-phenyl}-propionic acid

The procedure from Example 291, Step E is utilized with (S)-3-{4-[3-(2′-fluoro-5-trifluoromethyl-biphenyl-2-yloxy)-butoxy]-2-methyl-phenyl}-propionic acid methyl ester to afford 0.135 g (94%) of product. ¹H NMR (400 MHz, CDCl₃); MS (ES⁺) m/z mass calcd for C₂₇H₂₆F₄O₄ 490, found 508 (M+NH₄, 100%); MS (ES⁻) found 489 (M−1, 100%).

### EXAMPLE 302

### (S)-3-{4-[3-(2′-Methoxy-5-Trifluoromethyl-biphenyl-2-yloxy)-butoxy]-2-methyl-phenyl}-propionic acid

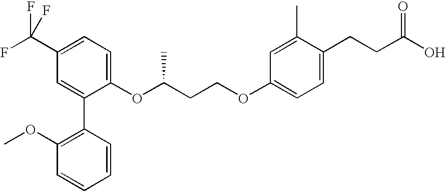

### Step A

### (S)-3-{4-[3-(2′-Methoxy-5-trifluoromethyl-biphenyl-2-yloxy)-butoxy]-2-methyl-phenyl}-propionic acid methyl ester

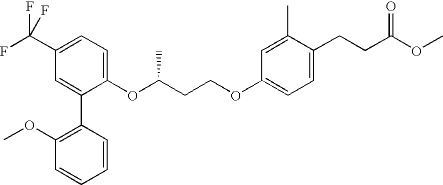

The procedure from Example 291, Step D is utilized with (S)-3-{4-[3-(2-bromo-4-trifluoromethyl-phenoxy)-butoxy]-2-methyl-phenyl}-propionic acid methyl ester and 2-methoxyphenyl boronic acid to afford 0.068 g (64%). ¹H NMR (400 MHz, CDCl₃); MS (ES⁺) m/z mass calcd for C₂₉H₃₁F₃O₅ 516, found 534 (M+NH₄, 100%).

### Step B

### (S)-3-{4-[3-(2′-Methoxy-5-Trifluoromethyl-biphenyl-2-yloxy)-butoxy]-2-methyl-phenyl}-propionic acid

The procedure from Example 291, Step E is utilized with (S)-3-{4-[3-(2′-methoxy-5-trifluoromethyl-biphenyl-2-yloxy)-butoxy]-2-methyl-phenyl}-propionic acid methyl ester to afford 0.06 g (91%) of product. ¹H NMR (400 MHz, CDCl₃); MS (ES⁺) m/z mass calcd for C₂₈H₂₉F₃O₅ 502, found 520 (M+NH₄, 100%); MS (ES⁻) found 501 (M−1, 100%).

### EXAMPLE 303

### (S)-3-{2-Ethyl-4-[3-(2′-fluoro-5-trifluoromethyl-biphenyl-2-yloxy)-butoxy]-phenyl}-propionic acid

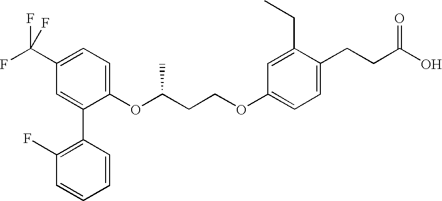

### Step A

### (S)-3-{4-[3-(2-Bromo-4-trifluoromethyl-phenoxy)-butoxy]-2-methyl-phenyl}-propionic acid methyl ester

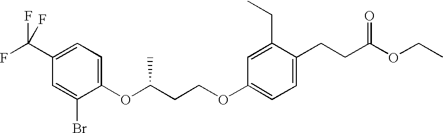

The procedure from Example 290, Step A is utilized with (R)-3-[2-ethyl-4-(3-methanesulfonyloxy-butoxy)-phenyl]-propionic acid ethyl ester and 2-bromo-4-trifluoromethyl-phenol to afford 0.5 g (69%) of product. ¹H NMR (400 MHz, CDCl₃); MS (ES⁺) m/z mass calcd for C₂₄H₂₈BrF₃O₄ 516, found 534 (M+NH₄, 100%).

### Step B

### (S)-3-{2-Ethyl-4-[3-(2′-fluoro-5-trifluoromethyl-biphenyl-2-yloxy)-butoxy]-phenyl}-propionic acid ethyl ester

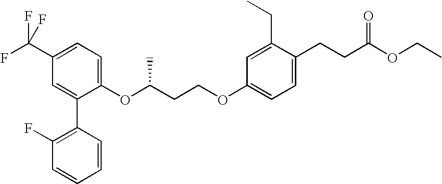

The procedure from Example 291, Step D is utilized with (S)-3-{4-[3-(2-bromo-4-trifluoromethyl-phenoxy)-butoxy]-2-methyl-phenyl}-propionic acid methyl ester to afford 0.104 g (68%). ¹H NMR (400 MHz, CDCl₃); MS (ES⁺) m/z mass calcd for C₃₀H₃₂F₄O₄ 532, found 550 (M+NH₄, 100%).

### Step C

### (S)-3-{2-Ethyl-4-[3-(2′-fluoro-5-trifluoromethyl-biphenyl-2-yloxy)-butoxy]-phenyl}-propionic acid

The procedure from Example 291, Step E is utilized with (S)-3-{2-ethyl-4-[3-(2′-fluoro-5-trifluoromethyl-biphenyl-2-yloxy)-butoxy]-phenyl}-propionic acid ethyl ester to afford 0.096 g (97%) of product. ¹H NMR (400 MHz, CDCl₃); MS (ES⁺) m/z mass calcd for C₂₈H₂₈F₄O₄ 504, found 522 (M+NH₄, 100%); MS (ES⁻) found 503 (M−1, 100%).

### EXAMPLE 304

### (S)-3-{2-Ethyl-4-[3-(3′-fluoro-5-trifluoromethyl-biphenyl-2-yloxy)-butoxy]-phenyl}-propionic acid

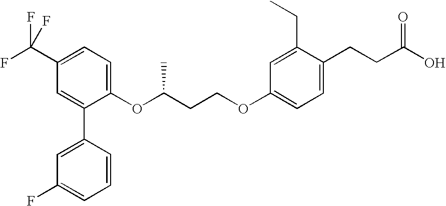

### Step A

### (S)-3-{2-Ethyl-4-[3-(3′-fluoro-5-trifluoromethyl-biphenyl-2-yloxy)-butoxy]-phenyl}-propionic acid ethyl ester

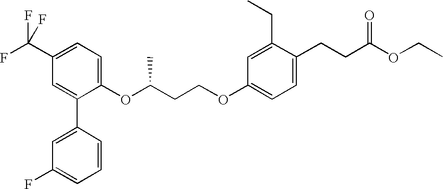

The procedure from Example 291, Step D is utilized with (S)-3-{4-[3-(2-bromo-4-trifluoromethyl-phenoxy)-butoxy]-2-methyl-phenyl}-propionic acid methyl ester and 3-Fluorophenylboronic acid to afford 0.115 g (75%). ¹H NMR (400 MHz, CDCl₃); MS (ES⁺) m/z mass calcd for C₃₀H₃₂F₄O₄ 532, found 550 (M+NH₄, 100%).

### Step B

### (S)-3-{2-Ethyl-4-[3-(3′-fluoro-5-trifluoromethyl-biphenyl-2-yloxy)-butoxy]-phenyl}-propionic acid

The procedure from Example 291, Step E is utilized with (S)-3-{2-ethyl-4-[3-(3′-fluoro-5-trifluoromethyl-biphenyl-2-yloxy)-butoxy]-phenyl}-propionic acid ethyl ester to afford 0.104 g (95%) of product. ¹H NMR (400 MHz, CDCl₃); MS (ES⁺) m/z mass calcd for C₂₈H₂₈F₄O₄ 504, found 522 (M+NH₄, 100%); MS (ES⁻) found 503 (M−1, 100%).

### EXAMPLE 305

### (S)-3-{2-Ethyl-4-[3-(4′-fluoro-5-trifluoromethyl-biphenyl-2-yloxy)-butoxy]-phenyl}-propionic acid

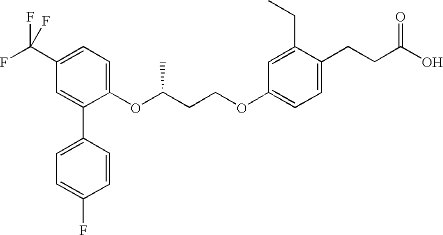

### Step A

### (S)-3-{2-Ethyl-4-[3-(4′-fluoro-5-trifluoromethyl-biphenyl-2-yloxy)-butoxy]-phenyl}-propionic acid ethyl ester

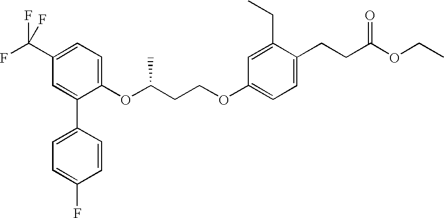

The procedure from Example 291, Step D is utilized with (S)-3-{4-[3-(2-bromo-4-trifluoromethyl-phenoxy)-butoxy]-2-methyl-phenyl}-propionic acid methyl ester and 4-fluorophenylboronic acid to afford 0.131 g (85%). ¹H NMR (400 MHz, CDCl₃); MS (ES⁺) m/z mass calcd for C₃₀H₃₂F₄O₄ 532, found 550 (M+NH₄, 100%).

### Step B

### (S)-3-{2-Ethyl-4-[3-(4′-fluoro-5-trifluoromethyl-biphenyl-2-yloxy)-butoxy]-phenyl}-propionic acid

The procedure from Example 291, Step E is utilized with (S)-3-{2-ethyl-4-[3-(4′-fluoro-5-trifluoromethyl-biphenyl-2-yloxy)-butoxy]-phenyl}-propionic acid ethyl ester to afford 0.113 g (91%) of product. ¹H NMR (400 MHz, CDCl₃); MS (ES⁺) m/z mass calcd for C₂₈H₂₈F₄O₄ 504, found 522 (M+NH₄, 100%); MS (ES⁻) found 503 (M−1, 100%).

### EXAMPLE 306

### (S)-3-{2-Ethyl-4-[3-(5-trifluoromethyl-biphenyl-2-yloxy)-butoxy]-phenyl}-propionic acid

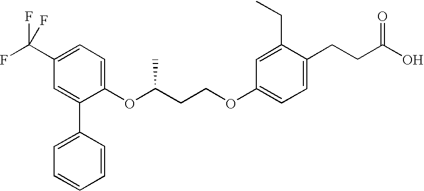

### Step A

### (S)-3-{2-Ethyl-4-[3-(5-trifluoromethyl-biphenyl-2-yloxy)-butoxy]-phenyl}-propionic acid ethyl ester

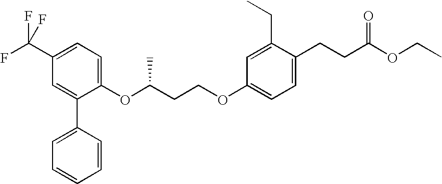

The procedure from Example 291, Step D is utilized with (S)-3-{4-[3-(2-bromo-4-trifluoromethyl-phenoxy)-butoxy]-2-methyl-phenyl}-propionic acid methyl ester and Phenylboronic acid to afford 0.068 g (68%). ¹H NMR (400 MHz, CDCl₃); MS (ES⁺) m/z mass calcd for C₃₀H₃₃F₃O₄ 514, found 532 (M+NH₄, 100%).

### Step B

### (S)-3-{2-Ethyl-4-[3-(5-trifluoromethyl-biphenyl-2-yloxy)-butoxy]-phenyl}-propionic acid

The procedure from Example 291, Step E is utilized with (S)-3-{2-ethyl-4-[3-(5-trifluoromethyl-biphenyl-2-yloxy)-butoxy]-phenyl}-propionic acid ethyl ester to afford 0.059 g (92%) of product. ¹H NMR (400 MHz, CDCl₃); MS (ES⁺) m/z mass calcd for C₂₈H₂₉F₃O₄ 486, found 504 (M+NH₄, 100%); MS (ES⁻) found 485 (M−1, 100%).

### EXAMPLE 307

### (S)-3-{2-Ethyl-4-[3-(3′-methoxy-4-fluoromethyl-biphenyl-2-yloxy)-butoxy]-phenyl}-propionic acid

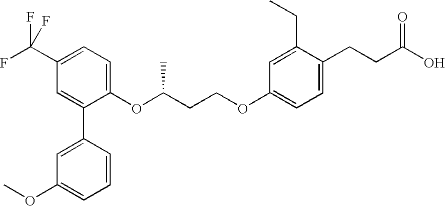

A solution of (S)-3-{4-[3-(2-bromo-4-trifluoromethyl-phenoxy)-butoxy]-2-methyl-phenyl}-propionic acid methyl ester (0.1 g, 0.19 mmol), 3-methoxyphenyl-boronic acid (0.088 g, 0.58 mmol), cesium fluoride (0.103 g, 0.68 mmol), and 1,1′-bis(diphenylphosphino)ferrocene palladium(II)chloride complex with dichloromethane (0.028 g, 0.04 mmol) in acetonitrile (3 mL) is purged with nitrogen. The reaction is heated to reflux and stirred overnight. The reaction is then treated with 5N aqueous sodium hydroxide (0.5 mL) and stirred for an additional 2 hr. The reaction is cooled and quenched with 1N aqueous hydrochloric acid to pH=4. The aqueous solution is extracted with diethyl ether. The organic is washed with brine and dried over sodium sulfate. The organic is filtered and the solvent is removed to afford the crude product. The crude is purified by reverse phase HPLC. The solvent is removed to afford 0.027 g (27%) of product. ¹H NMR (400 MHz, CDCl₃); MS (ES⁻) m/z mass calcd for C₂₉H₃₁F₃O₅ 516, found 515 (M−1, 100%).

### EXAMPLE 308

### (S)-3-{2-Ethyl-4-[3-(4′-methoxy-5-trifluoromethyl-biphenyl-2-yloxy)-butoxy]-phenyl}-propionic acid

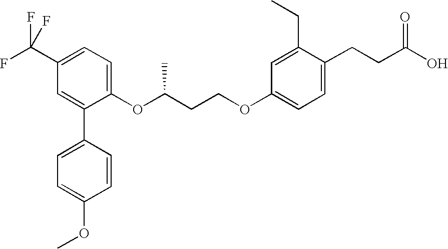

The procedure from Example 307 is utilized with 4-methoxyphenylboronic acid to afford 0.034 g (34%) of product. ¹H NMR (400 MHz, CDCl₃); MS (ES⁻) m/z mass calcd for C₂₉H₃₁F₃O₅ 516, found 515 (M−1, 100%).

### EXAMPLE 309

### (S)-3-(4-{3-[2-(2,3-Dihydro-benzo[1,4]dioxin-6-yl)-4-trifluoromethyl-phenoxy]-butoxy}-2-ethyl-phenyl)-propionic acid

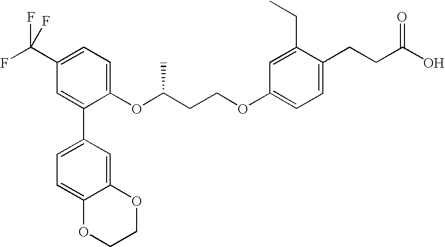

The procedure from Example 307 is utilized with 1,4-benzodioxane-6-boronic acid to afford 0.059 g (56%) of product. ¹H NMR (400 MHz, CDCl₃); MS (ES⁻) m/z mass calcd for C₃₀H₃₁F₃O₆ 544, found 543 (M−1, 100%).

### EXAMPLE 310

### (S)-3-{4-[3-(2′-Chloro-5-trifluoromethyl-biphenyl-2-yloxy)-butoxy]-2-ethyl-phenyl}-propionic acid

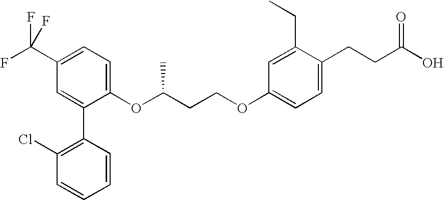

The procedure from Example 307 is utilized with 2-chlorophenylboronic acid to afford 0.042 g (41%) of product. ¹H NMR (400 MHz, CDCl₃); MS (ES⁻) m/z mass calcd for C₂₈H₂₈ClF₃O₄ 520, found 519 (M−1, 100%).

### EXAMPLE 311

### (S)-3-{4-[3-(3′-Chloro-5-trifluoromethyl-biphenyl-2-yloxy)-butoxy]-2-ethyl-phenyl}-propionic acid

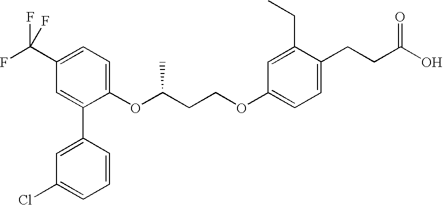

The procedure from Example 307 is utilized with 3-chlorophenylboronic acid to afford 0.035 g (35%) of product. ¹H NMR (400 MHz, CDCl₃); MS (ES⁻) m/z mass calcd for C₂₈H₂₈ClF₃O₄ 520, found 519 (M−1, 100%).

### EXAMPLE 312

### (S)-3-{4-[3-(4′-Chloro-5-trifluoromethyl-biphenyl-2-yloxy)-butoxy]-2-ethyl-phenyl}-propionic acid

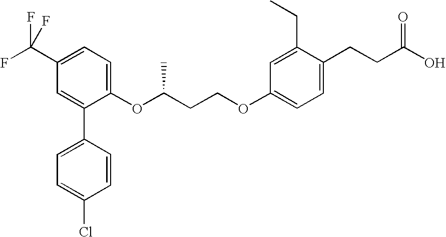

The procedure from Example 307 is utilized with 4-chlorophenylboronic acid to afford 0.052 g (52%) of product. ¹H NMR (400 MHz, CDCl₃); MS (ES⁻) m/z mass calcd for C₂₈H₂₈ClF₃O₄ 520, found 519 (M−1, 100%).

### EXAMPLE 313

### (S)-3-{4-[3-(4′-Dimethylamino-5-trifluoromethyl-biphenyl-2-yloxy)-butoxy]-2-ethyl-phenyl}-propionic acid

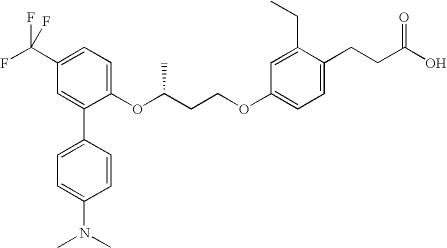

The procedure from Example 307 is utilized with 4-Dimethylaminophenyl boronic acid to afford 0.046 g (46%) of product. ¹H NMR (400 MHz, CDCl₃); MS (ES⁻) m/z mass calcd for C₃₀H₃₄F₃NO₄ 529, found 528 (M−1, 100%).

### EXAMPLE 314

### (S)-3-{4-[3-(5,2′-Bis-trifluoromethyl-biphenyl-2-yloxy)-butoxy]-2-ethyl-phenyl}-propionic acid

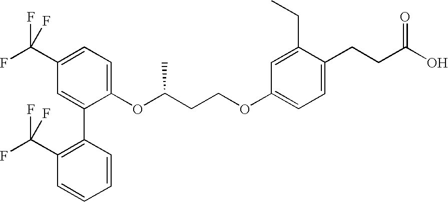

The procedure from Example 307 is utilized with 2-trifluoromethyl-phenylboronic acid to afford 0.02 g (19%) of product. ¹H NMR (400 MHz, CDCl₃); MS (ES−) m/z mass calcd for C₂₉H₂₈F₆O₄ 554, found 553 (M−1, 100%).

### EXAMPLE 315

### (S)-3-{4-[3-(5,3-Bis-trifluoromethyl-biphenyl-2-yloxy)-butoxy]-2-ethyl-phenyl}-propionic acid

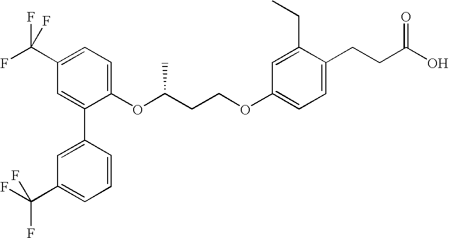

The procedure from Example 307 is utilized with 3-trifluoromethyl-phenylboronic acid to afford 0.049 g (45%) of product. ¹H NMR (400 MHz, CDCl₃); MS (ES−) m/z mass calcd for C₂₉H₂₈F₆O₄ 554, found 553 (M−1, 100%).

### EXAMPLE 316

### (S)-3-{4-[3-(5,4′-Bis-trifluoromethyl-biphenyl-2-yloxy)-butoxy]-2-ethyl-phenyl}-propionic acid

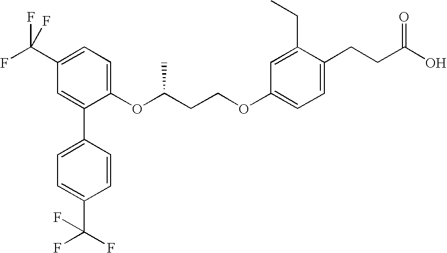

The procedure from Example 307 is utilized with 4-trifluoromethyl-phenylboronic acid to afford 0.07 g (65%) of product. ¹H NMR (400 MHz, CDCl₃); MS (ES−) m/z mass calcd for C₂₉H₂₈F₆O₄ 554, found 553 (M−1, 100%).

### EXAMPLE 317

### (S)-3-{2-Ethyl-4-[3-(4′-methanesulfonyl-5-trifluoromethyl-biphenyl-2-yloxy)-butoxy]-phenyl}-propionic acid

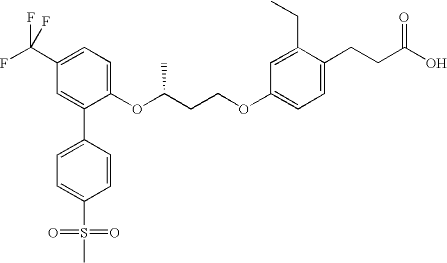

The procedure from Example 307 is utilized with (4-methylsulfonylphenyl)boronic acid to afford 0.021 g (19%) of product. ¹H NMR (400 MHz, CDCl₃); MS (ES−) m/z mass calcd for C₂₉H₃₁F₃O₆S 564, found 563 (M−1, 100%).

### EXAMPLE 318

### (S)-3-{6-[3-(4-Chloro-2-phenoxy-phenoxy)-butoxy]-4-methyl-pyridin-3-yl}-propionic acid

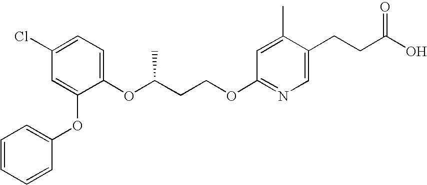

A solution of (S)-3-methanesulfonic acid 3-(4-chloro-2-phenoxy-phenoxy)-butyl ester (0.177 g, 0.48 mmol) and 3-(6-hydroxy-4-methyl-pyridin-3-yl)-propionic acid ethyl ester (0.1 g, 0.48 mmol) in DMF (5 mL) is treated with cesium carbonate (0.171 g, 0.53 mmol). The reaction is heated to 60° C. and stirred overnight. The reaction is then treated with 5N aqueous sodium hydroxide (0.4 mL) and stirred for an additional 2 hr. The reaction is cooled and quenched with 1N aqueous hydrochloric acid to pH=7. The aqueous solution is extracted with diethyl ether. The organic is washed with brine and dried over sodium sulfate. The organic is filtered and the solvent is removed to afford the crude product. The crude is purified by reverse phase HPLC. The solvent is removed to afford 0.072 g (33%) of product. ¹H NMR (400 MHz, CDCl₃); MS (ES+) m/z mass calcd for C₂₅H₂₆ClNO₅ 455, found 456 (M+1, 100%); MS (ES−) found 454 (M−1, 100%).

### EXAMPLE 319

### (S)-3-{6-[3-(4-Ethyl-2-phenoxy-phenoxy)-butoxy]-4-methyl-pyridin-3-yl}-propionic acid

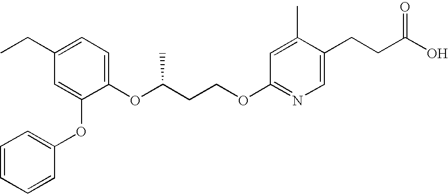

The procedure from Example 318 is utilized with (S)-methanesulfonic acid 3-(4-ethyl-2-phenoxy-phenoxy)-butyl ester to afford 0.077 g (36%) of desired product. ¹H NMR (400 MHz, CDCl₃); MS (ES+) m/z mass calcd for C₂₇H₃₁NO₅ 449, found 450 (M+1, 100%); MS (ES−) found 448 (M−1, 100%).

### EXAMPLE 320

### (S)-3-{4-Methyl-6-[3-(2-phenoxy-4-trifluoromethyl-phenoxy)-butoxy]-pyridin-3-yl}-propionic acid

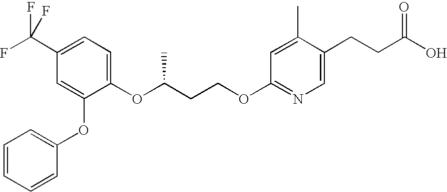

The procedure from Example 318 is utilized with (S)-methanesulfonic acid 3-(2-phenoxy-4-trifluoromethyl-phenoxy)-butyl ester to afford 0.096 g (41%) of desired product. ¹H NMR (400 MHz, CDCl₃); MS (ES⁺) m/z mass-calcd for C₂₆H₂₆F₃NO₅ 489, found 490 (M+1, 100%); MS (ES⁻) found 488 (M−1, 100%).

### EXAMPLE 321

### (S)-3-{6-[3-(2-Benzoyl-4-ethyl-phenoxy)-butoxy]-4-methyl-pyridin-3-yl}-propionic acid

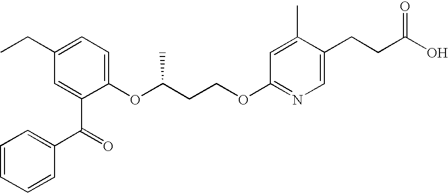

The procedure from Example 318 is utilized with (S)-methanesulfonic acid 3-(2-benzoyl-4-ethyl-phenoxy)-butyl ester to afford 0.064 g (29%) of desired product. ¹H NMR (400 MHz, CDCl₃); MS (ES+) m/z mass calcd for C₂₈H₃₁NO₅ 461, found 462 (M+1, 100%); MS (ES−) found 460 (M−1, 100%).

### EXAMPLE 322

### (S)-{3-[3-(2-Phenoxy-4-trifluoromethyl-phenoxy)-butoxy]-phenyl}-acetic acid

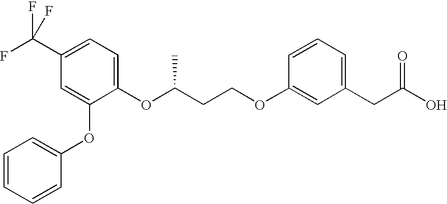

The procedure from Example 284, Step C is utilized with (S)-methanesulfonic acid 3-(2-phenoxy-4-trifluoromethyl-phenoxy)-butyl ester and (3-hydroxy-phenyl)-acetic acid methyl ester to afford 0.072 g (63%) of desired product. ¹H NMR (400 MHz, CDCl₃); MS (ES⁺) m/z mass calcd for C₂₅H₂₃F₃O₅ 460, found 478 (M+NH₄, 100%); MS (ES⁻) found 459 (M−1, 100%).

### EXAMPLE 323

### (S)-3-{3-[3-(2-Phenoxy-4-trifluoromethyl-phenoxy)-butoxy]-phenyl}-propionic acid

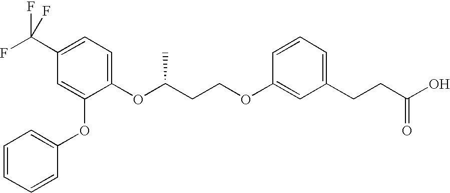

The procedure from Example 284, Step C is utilized with (S)-methanesulfonic acid 3-(2-phenoxy-4-trifluoromethyl-phenoxy)-butyl ester and 3-(3-hydroxy-phenyl)-propionic acid methyl ester to afford 0.076 g (65%) of desired product. ¹H NMR (400 MHz, CDCl₃); MS (ES⁺) m/z mass calcd for C₂₆H₂₅F₃O₅ 474, found 492 (M+NH₄, 100%); MS (ES⁻) found 473 (M−1, 100%).

### EXAMPLE 324

### (S)-3-{4-[3-(4-Chloro-2-phenoxy-phenoxy)-butoxy]-2-isopropyl-phenyl}-propionic acid

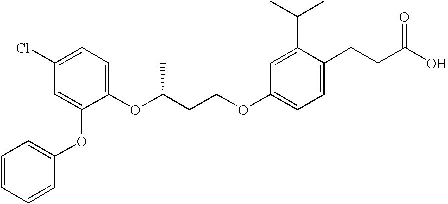

### Step A

### (S)-3-{4-[3-(4-Chloro-2-phenoxy-phenoxy)-butoxy]-2-isopropyl-phenyl}-propionic acid ethyl ester

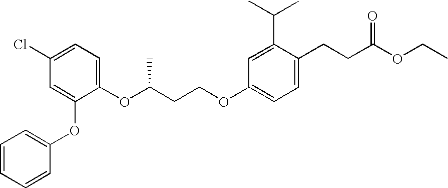

The procedure from Example 290, Step A is utilized with (S)-methanesulfonic acid 3-(4-chloro-2-phenoxy-phenoxy)-butyl ester and 3-(4-hydroxy-2-isopropyl-phenyl)-propionic acid ethyl ester to afford 0.122 g (54%) of product. ¹H NMR (400 MHz, CDCl₃); MS (ES⁺) m/z mass calcd for C₃₀H₃₅ClO₅ 510, found 528 (M+NH₄, 100%).

### Step B

### (S)-3-{4-[3-(4-Chloro-2-phenoxy-phenoxy)-butoxy]-2-isopropyl-phenyl}-propionic acid

The procedure from Example 294, Step B is utilized with (S)-3-{4-[3-(4-chloro-2-phenoxy-phenoxy)-butoxy]-2-isopropyl-phenyl}-propionic acid ethyl ester to afford 0.109 g (95%) of product. ¹H NMR (400 MHz, CDCl₃); MS (ES⁺) m/z mass calcd for C₂₈H₃₁ClO₅ 482, found 500 (M+NH₄, 100%); MS (ES−) found 481 (M−1, 100%).

### EXAMPLE 325

### (S)-3-{5-[3-(4-Chloro-2-phenoxy-phenoxy)-butoxy]-3-methyl-pyridin-2-yl}-propionic acid

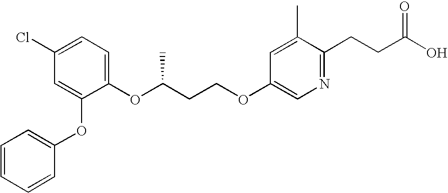

### Step A

### (S)-3-{5-[3-(4-Chloro-2-phenoxy-phenoxy)-butoxy]-3-methyl-pyridin-2-yl}-propionic acid ethyl ester

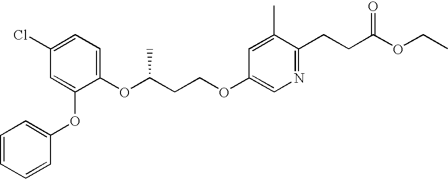

The procedure from Example 290, Step A is utilized with (S)-methanesulfonic acid 3-(4-chloro-2-phenoxy-phenoxy)-butyl ester and 3-(5-Hydroxy-3-methyl-pyridin-2-yl)-propionic acid ethyl ester to afford 0.062 g (310%) of product. ¹H NMR (400 MHz, CDCl₃); MS (ES⁺) m/z mass calcd for C₂₇H₃₀ClNO₅ 483, found 484 (M+1, 100%).

### Step B

### (S)-3-{5-[3-(4-Chloro-2-phenoxy-phenoxy)-butoxy]-3-methyl-pyridin-2-yl}-propionic acid

The procedure from Example 294, Step B is utilized with (S)-3-{5-[3-(4-chloro-2-phenoxy-phenoxy)-butoxy]-3-methyl-pyridin-2-yl}-propionic acid ethyl ester to afford 0.022 g (38%) of product. ¹H NMR (400 MHz, CDCl₃); MS (ES⁺) m/z mass calcd for C₂₅H₂₆ClNO₅ 455, found 456 (M+1, 100%); MS (ES−) found 454 (M−1,20%).

### EXAMPLE 326

### (R)-3-{4-[3-(4-Chloro-2-phenoxy-phenoxy)-1-methyl-propoxy]-2-ethyl-phenyl}-propionic acid

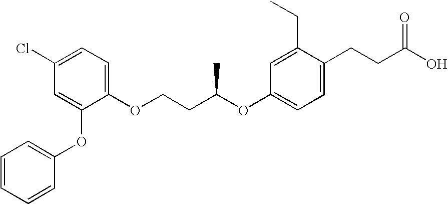

### Step A

### (R)-4-(4-Chloro-2-phenoxy-phenoxy)-butan-2-ol

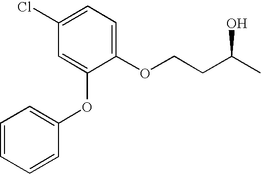

The procedure from Example 284, Step A is utilized with (R)-toluene-4-sulfonic acid 3-hydroxy-butyl ester and 4-Chloro-2-phenoxy-phenol to afford 0.73 g (61%) of product. ¹H NMR (400 MHz, CDCl₃); MS (ES⁺) m/z mass calcd for C₁₆H₁₇ClO₃ 292, found 293 (M+1,70%), 310 (M+NH₄, 100%).

### Step B

### (R)-Methanesulfonic acid 3-(4-chloro-2-phenoxy-phenoxy)-1-methyl-propyl ester

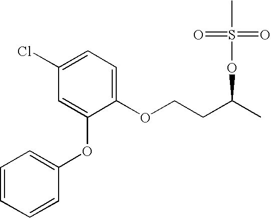

The procedure from Example 284, Step B is utilized with (R)-4-(4-chloro-2-phenoxy-phenoxy)-butan-2-ol to afford 0.84 g (92%) of product. ¹H NMR (400 MHz, CDCl₃); MS (ES⁺) m/z mass calcd for C₁₇H₁₉ClO₅S 370, found 388 (M+NH₄, 100%).

### Step C

### (R)-3-{4-[3-(4-Chloro-2-phenoxy-phenoxy)-1-methyl-propoxy]-2-ethyl-phenyl}-propionic acid ethyl ester

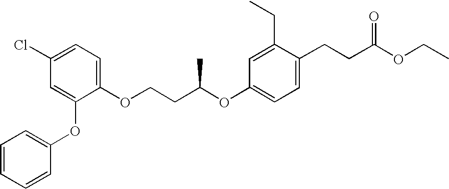

The procedure from Example 290, Step A is utilized with (R)-methanesulfonic acid 3-(4-chloro-2-phenoxy-phenoxy)-1-methyl-propyl ester and 3-(2-ethyl-4-hydroxy-phenyl)-propionic acid ethyl ester to afford 0.074 g (55%) of product. ¹H NMR (400 MHz, CDCl₃); MS (ES⁺) m/z mass calcd for C₂₉H₃₃ClO₅ 496, found 514 (M+NH₄, 100%).

### Step D

### (R)-3-{4-[3-(4-Chloro-2-phenoxy-phenoxy)-1-methyl-propoxy]-2-ethyl-phenyl}-propionic acid

The procedure from Example 294, Step B is utilized with (R)-3-{4-[3-(4-chloro-2-phenoxy-phenoxy)-1-methyl-propoxy]-2-ethyl-phenyl}-propionic acid ethyl ester to afford 0.074 g (100%) of product. ¹H NMR (400 MHz, CDCl₃); MS (ES⁺) m/z mass calcd for C₂₇H₂₉ClO₅ 468, found 586 (M+NH₄, 100%); MS (ES⁻) found 467 (M−H, 100%).

### EXAMPLE 327

### (R)-3-{4-[3-(4-Chloro-2-phenoxy-phenoxy)-1-methyl-propoxy]-2-ethyl-phenyl}-propionic acid

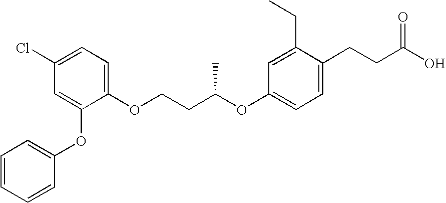

### Step A

### (S)-4-(4-Chloro-2-phenoxy-phenoxy)-butan-2-ol

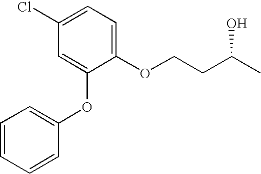

The procedure from Example 284, Step A is utilized with (S)-toluene-4-sulfonic acid 3-hydroxy-butyl ester and 4-chloro-2-phenoxy-phenol to afford 0.78 g (65%) of product. ¹H NMR (400 MHz, CDCl₃); MS (ES⁺) m/z mass calcd for C₁₆H₁₇ClO₃ 292, found 293 (M+1, 70%), 310 (M+NH₄, 100%).

### Step B

### (S)-Methanesulfonic acid 3-(4-chloro-2-phenoxy-phenoxy)-1-methyl-propyl ester

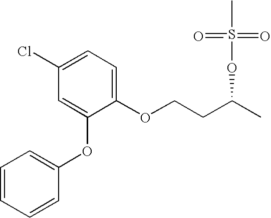

The procedure from Example 284, Step B is utilized with (S)-4-(4-chloro-2-phenoxy-phenoxy)-butan-2-ol to afford 0.86 g (87%) of product. ¹H NMR (400 MHz, CDCl₃); MS (ES⁺) m/z mass calcd for C₁₇H₁₉ClO₅S 370, found 388 (M+NH₄, 100%).

### Step C

### (R)-3-{4-[3-(4-Chloro-2-phenoxy-phenoxy)-1-methyl-propoxy]-2-ethyl-phenyl}-propionic acid ethyl ester

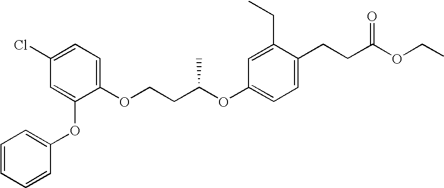

The procedure from Example 290, Step A is utilized with (S)-methanesulfonic acid 3-(4-chloro-2-phenoxy-phenoxy)-1-methyl-propyl ester and 3-(2-ethyl-4-hydroxy-phenyl)-propionic acid ethyl ester to afford 0.056 g (42%) of product. ¹H NMR (400 MHz, CDCl₃); MS (ES⁺) m/z mass calcd for C₂₉H₃₃ClO₅ 496, found 514 (M+NH₄, 100%).

### Step D

### (R)-3-{4-[3-(4-Chloro-2-phenoxy-phenoxy)-1-methyl-propoxy]-2-ethyl-phenyl}-propionic acid

The procedure from Example 294, Step B is utilized with (R)-3-{4-[3-(4-chloro-2-phenoxy-phenoxy)-1-methyl-propoxy]-2-ethyl-phenyl}-propionic acid ethyl ester to afford 0.05 g (94%) of product. ¹H NMR (400 MHz, CDCl₃); MS (ES⁺) m/z mass calcd for C₂₇H₂₉ClO₅ 468, found 586 (M+NH₄, 100%); MS (ES−) found 467 (M−H, 100%).

### EXAMPLE 328

### (S)-3-(4-{3-[4-Chloro-2-(3-fluoro-phenoxy)-phenoxy]-butoxy}-2-ethyl-phenyl)-propionic acid

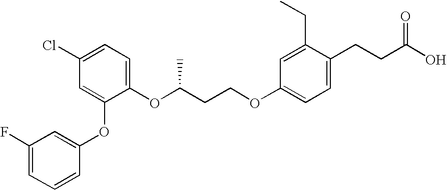

### Step A

### (S)-3-(4-{3-[4-Chloro-2-(3-fluoro-phenoxy)-phenoxy]-butoxy}-2-ethyl-phenyl)-propionic acid ethyl ester

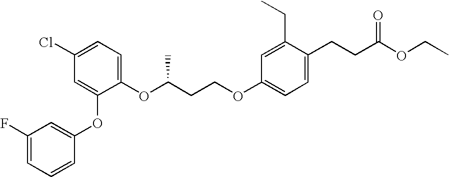

The procedure from Example 290, Step A is utilized with 4-chloro-2-(3-fluoro-phenoxy)-phenol and 3-[4-(3-methanesulfonyloxy-butoxy)-2-ethyl-phenyl]-propionic acid ethyl ester to afford 0.095 g (69%) of product. ¹H NMR (400 MHz, CDCl₃). MS (ES⁺) m/z mass calcd for C₂₉H₃₂ClFO₅ 514, found 532 (M+NH₄, 100%).

### Step B

### (S)-3-(4-{3-[4-Chloro-2-(3-fluoro-phenoxy)-phenoxy]-butoxy}-2-ethyl-phenyl)-propionic acid

The procedure from Example 291, Step E is utilized with (S)-3-(4-{3-[4-chloro-2-(3-fluoro-phenoxy)-phenoxy]-butoxy}-2-ethyl-phenyl)-propionic acid ethyl ester. The reaction affords 0.074 g (82%) of product. ¹H NMR (400 MHz, CDCl₃). MS (ES⁺) m/z mass calcd for C₂₇H₂₈ClFO₅ 486, found 504 (M+NH₄, 100%); MS (ES−) found 485.

### EXAMPLE 329

### (S)-3-(4-{3-[4-Chloro-2-(4-fluoro-phenoxy)-phenoxy]-butoxy}-2-ethyl-phenyl)-propionic acid

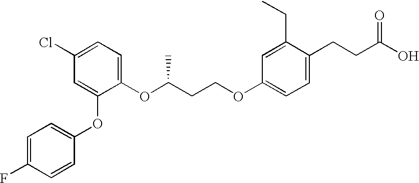

### Step A

### (S)-3-(4-{3-[4-Chloro-2-(4-fluoro-phenoxy)-phenoxy]-butoxy}-2-ethyl-phenyl)-propionic acid ethyl ester

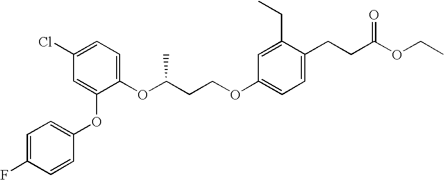

The procedure from Example 290, Step A is utilized with 4-chloro-2-(4-fluoro-phenoxy)-phenol and 3-[4-(3-methanesulfonyloxy-butoxy)-2-ethyl-phenyl]-propionic acid ethyl ester to afford 0.174 g (63%) of product. ¹H NMR (400 MHz, CDCl₃). MS (ES⁺) m/z mass calcd for C₂₉H₃₂ClFO₅ 514, found 532 (M+NH₄, 100%).

### Step B

### (S)-3-(4-{3-[4-Chloro-2-(4-fluoro-phenoxy)-phenoxy]-butoxy}-2-ethyl-phenyl)-propionic acid

The procedure from Example 291, Step E is utilized with (S)-3-(4-{3-[4-chloro-2-(4-fluoro-phenoxy)-phenoxy]-butoxy}-2-ethyl-phenyl)-propionic acid ethyl ester. The reaction affords 0.147 g (90%) of product. ¹H NMR (400 MHz, CDCl₃). MS (ES⁺) m/z mass calcd for C₂₇H₂₈ClFO₅ 486, found 504 (M+NH₄, 100%); MS (ES−) found 485.

### EXAMPLE 330

### (S)-3-{3-Methyl-5-[3-(2-phenoxy-4-trifluoromethyl-phenoxy)-butoxy]-pyridin-2-yl}-propionic acid

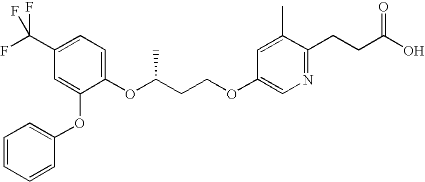

The procedure from Example 284, Step C is utilized with (S)-methanesulfonic acid 3-(2-phenoxy-4-trifluoromethyl-phenoxy)-butyl ester and 3-(5-hydroxy-3-methyl-pyridin-2-yl)-propionic acid ethyl ester to afford 0.092 g (51%) of product. ¹H NMR (400 MHz, CDCl₃); MS (ES⁺) m/z mass calcd for C₂₆H₂₆F₃NO₅ 489, found 490 (M+1, 100%); MS (ES−) found 488 (M−1, 20%).

### EXAMPLE 331

### (S)-3-{4-[3-(4-Chloro-2-o-tolyloxy-phenoxy)-butoxy]-2-ethyl-phenyl}-propionic acid

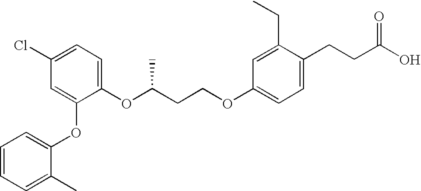

### Step A

### 4-Chloro-2-o-tolyloxy-benzaldehyde

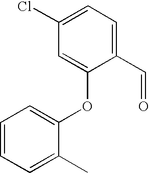

The procedure from Example 293, Step A is utilized with o-cresol to afford 1.09 g (70%) of product. ¹H NMR (400 MHz, CDCl₃), MS (ES+) m/z mass calcd for C₁₄H₁₁ClO₂ 246, found 247 (M+1, 100%).

### Step B

### 4-Chloro-2-o-tolyloxy-phenol

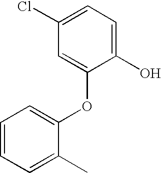

The procedure from Example 293, Step B is utilized with 4-chloro-2-o-tolyloxy-benzaldehyde to afford 0.539 g (52%) of product. ¹H NMR (400 MHz, CDCl₃). MS (ES−) m/z mass calcd for C₁₃H₁₁ClO₂ 234, found 233 (M−1, 100%).

### Step C

### (S)-3-{4-[3-(4-Chloro-2-o-tolyloxy-phenoxy)-butoxy]-2-ethyl-phenyl}-propionic acid

The procedure from Example 284, Step C is utilized with (R)-3-[2-ethyl-4-(3-methanesulfonyloxy-butoxy)-phenyl]-propionic acid ethyl ester and 4-chloro-2-o-tolyloxy-phenol to afford 0.068 g (53%) of product. ¹H NMR (400 MHz, CDCl₃). MS (ES+) m/z mass calcd for C₂₈H₃₁ClO₅ 482, found 500 (M+NH₄, 100%), MS (ES−) found 481 (M−1, 100%).

### EXAMPLE 332

### (S)-3-{4-[3-(4-Chloro-2-m-tolyloxy-phenoxy)-butoxy]-2-ethyl-phenyl}-propionic acid

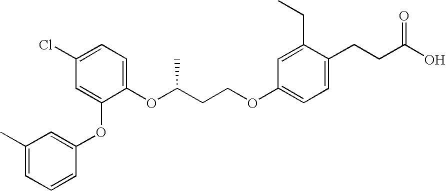

### Step A

### 4-Chloro-2-m-tolyloxy-benzaldehyde

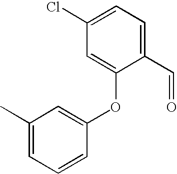

The procedure from Example 293, Step A is utilized with m-cresol to afford 1.18 g (76%) of product. ¹H NMR (400 MHz, CDCl₃), MS (ES+) m/z mass calcd for C₁₄H₁₁ClO₂ 246, found 247 (M+1, 100%).

### Step B

### 4-Chloro-2-m-tolyloxy-phenol

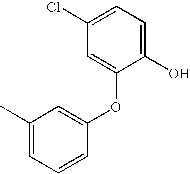

The procedure from Example 293, Step B is utilized with 4-chloro-2-m-tolyloxy-benzaldehyde to afford 0.581 g (52%) of product. ¹H NMR (400 MHz, CDCl₃). MS (ES−) m/z mass calcd for C₁₃H₁₁ClO₂ 234, found 233 (M−1, 100%).

### Step C

### (S)-3-{4-[3-(4-Chloro-2-n-tolyloxy-phenoxy)-butoxy]-2-ethyl-phenyl}-propionic acid

The procedure from Example 284, Step C is utilized with (R)-3-[2-ethyl-4-(3-methanesulfonyloxy-butoxy)-phenyl]-propionic acid ethyl ester and 4-chloro-2-m-tolyloxy-phenol to afford 0.079 g (61%) of product. ¹H NMR (400 MHz, CDCl₃). MS (ES+) m/z mass calcd for C₂₈H₃₁ClO₅ 482, found 500 (M+NH₄, 100%), MS (ES−) found 481 (M−1, 100%).

### EXAMPLE 333

### (S)-3-{4-[3-(4-Chloro-2-p-tolyloxy-phenoxy)-butoxy]-2-ethyl-phenyl}-propionic acid

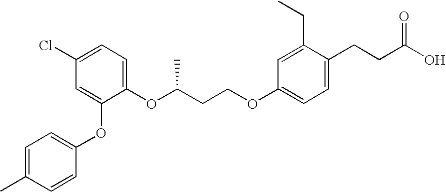

### Step A

### 4-Chloro-2-p-tolyloxy-benzaldehyde

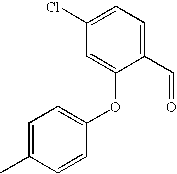

The procedure from Example 293, Step A is utilized with p-cresol to afford 1.02 g (65%) of product. ¹H NMR (400 MHz, CDCl₃), MS (ES+) m/z mass calcd for C₁₄H₁₁ClO₂ 246, found 247 (M+1, 100%).

### Step B

### 4-Chloro-2-p-tolyloxy-phenol

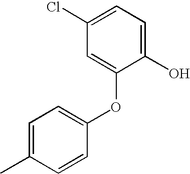

The procedure from Example 293, Step B is utilized with 4-chloro-2-p-tolyloxy-benzaldehyde to afford 0.408 g (42%) of product. ¹H NMR (400 MHz, CDCl₃). MS (ES−) m/z mass calcd for C₁₃H₁₁ClO₂ 234, found 233 (M−1, 100%).

### Step C

### (S)-3-{4-[3-(4-Chloro-2-p-tolyloxy-phenoxy)-butoxy]-2-ethyl-phenyl}-propionic acid

The procedure from Example 284, Step C is utilized with (R)-3-[2-ethyl-4-(3-methanesulfonyloxy-butoxy)-phenyl]-propionic acid ethyl ester and 4-chloro-2-p-tolyloxy-phenol to afford 0.078 g (60%) of product. ¹H NMR (400 MHz, CDCl₃). MS (ES+) m/z mass calcd for C₂₈H₃₁ClO₅ 482, found 500 (M+NH₄, 100%), MS (ES−) found 481 (M−1, 100%).

### EXAMPLE 334

### (S)-3-(4-{3-[4-Chloro-2-(2,4-difluoro-phenoxy)-phenoxy]-butoxy}-2-ethyl-phenyl)-propionic acid

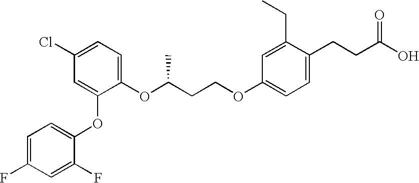

### Step A

### 4-Chloro-2-(2,4-difluoro-phenoxy)-benzaldehyde

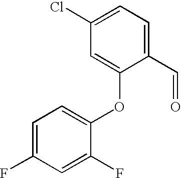

The procedure from Example 293, Step A is utilized with 2,4-difluorophenol to afford 1.69 g (100%) of product. ¹H NMR (400 MHz, CDCl₃), MS (ES+) m/z mass calcd for C₁₃H₇ClF₂O₂ 268, found 269 (M+1,30%).

### Step B

### 4-Chloro-2-(2,4-difluoro-phenoxy)-phenol

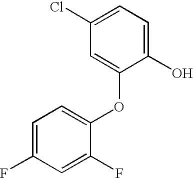

The procedure from Example 293, Step B is utilized with 4-chloro-2-(2,4-difluoro-phenoxy)-benzaldehyde to afford 0.739 g (46%) of product. ¹H NMR (400 MHz, CDCl₃). MS (ES−) m/z mass calcd for C₁₂H₇ClF₂O₂ 256, found 255 (M−1, 100%).

### Step C

### (S)-3-(4-{3-[4-Chloro-2-(2,4-difluoro-phenoxy)-phenoxy]-butoxy}-2-ethyl-phenyl)-propionic acid ethyl ester

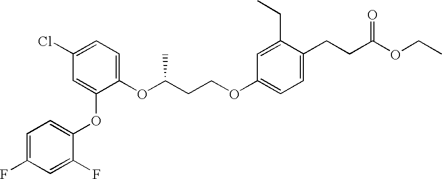

The procedure from Example 290, Step A is utilized with (R)-3-[2-ethyl-4-(3-methanesulfonyloxy-butoxy)-phenyl]-propionic acid ethyl ester and 4-chloro-2-(2,4-difluoro-phenoxy)-phenol to afford 0.044 g (15%) of product. ¹H NMR (400 MHz, CDCl₃). MS (ES+) m/z mass calcd for C₂₉H₃₁ClF₂O₅ 532, found 550 (M+NH₄, 100%).

### Step D

### (S)-3-(4-{3-[4-Chloro-2-(2,4-difluoro-phenoxy)-phenoxy]-butoxy}-2-ethyl-phenyl)-propionic acid

The procedure from Example 291, Step E is utilized with (S)-3-(4-{3-[4-chloro-2-(2,4-difluoro-phenoxy)-phenoxy]-butoxy}-2-ethyl-phenyl)-propionic acid ethyl ester to afford 0.033 g (79%) of product. ¹H NMR (400 MHz, CDCl₃). MS (ES+) m/z mass calcd for C₂₇H₂₇ClF₂O₅ 504, found 522 (M+NH₄, 100%), MS (ES−) found 503 (M−1, 100%).

### EXAMPLE 335

### (S)-3-(4-{3-[4-Chloro-2-(2,4-difluoro-phenoxy)-phenoxy]-butoxy}-2-ethyl-phenyl)-propionic acid

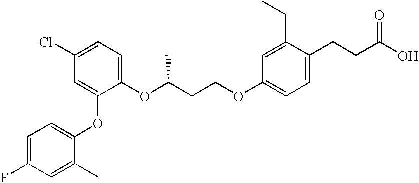

### Step A

### 4-Chloro-2-(4-fluoro-2-methyl-phenoxy)-benzaldehyde

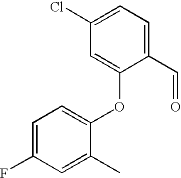

The procedure from Example 293, Step A is utilized with 4-fluoro-2-methylphenol to afford 1.68 g (100%) of product. ¹H NMR (400 MHz, CDCl₃), MS (ES+) m/z mass calcd for C₁₄H₁₀ClFO₂ 264, found 265 (M+1,30%).

### Step B

### 4-Chloro-2-(4-fluoro-2-methyl-phenoxy)-phenol

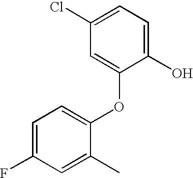

The procedure from Example 293, Step B is utilized with 4-chloro-2-(4-fluoro-2-methyl-phenoxy)-benzaldehyde to afford 1.07 g (66%) of product. ¹H NMR (400 MHz, CDCl₃). MS (ES−) m/z mass calcd for C₁₃H₁₀ClFO₂ 252, found 251 (M−1, 100%).

### Step C

### (S)-3-(4-{3-[4-Chloro-2-(4-fluoro-2-methyl-phenoxy)-phenoxy]-butoxy}-2-ethyl-phenyl)-propionic acid ethyl ester

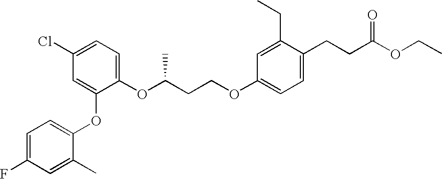

The procedure from Example 290, Step A is utilized with (R)-3-[2-ethyl-4-(3-methanesulfonyloxy-butoxy)-phenyl]-propionic acid ethyl ester and 4-chloro-2-(4-fluoro-2-methyl-phenoxy)-phenol to afford 0.208 g (73%) of product. ¹H NMR (400 MHz, CDCl₃). MS (ES+) m/z mass calcd for C₃₀H₃₄ClFO₅ 528, found 546 (M+NH₄, 100%).

### Step D

### (S)-3-(4-{3-[4-Chloro-2-(2,4-difluoro-phenoxy)-phenoxy]-butoxy}-2-ethyl-phenyl)-propionic acid

The procedure from Example 291, Step E is utilized with (S)-3-(4-{3-[4-chloro-2-(4-fluoro-2-methyl-phenoxy)-phenoxy]-butoxy}-2-ethyl-phenyl)-propionic acid ethyl ester to afford 0.190 g (96%) of product. ¹H NMR (400 MHz, CDCl₃). MS (ES+) m/z mass calcd for C₂₉H₃₀ClFO₅ 500, found 518 (M+NH₄, 100%), MS (ES−) found 499 (M−1, 100%).

### EXAMPLE 336

### (S)-{3-[3-(2-Pyrimidin-2-yl-4-trifluoromethyl-phenoxy)-butylsulfanyl]-phenyl}-acetic acid

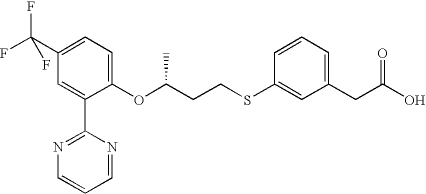

### Step A

### (S)-{3-[3-(2-Pyrimidin-2-yl-4-trifluoromethyl-phenoxy)-butylsulfanyl]-phenyl}-acetic acid methyl ester

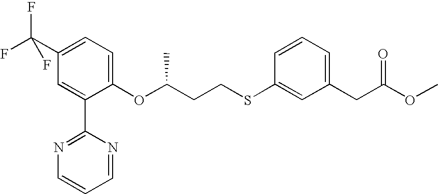

The procedure from Example 290, Step A is utilized with (R)-[3-(3-methanesulfonyloxy-butylsulfanyl)-phenyl]-acetic acid methyl ester and 2-pyrimidin-2-yl-4-trifluoromethyl-phenol to afford 0.114 g (79%) of product. ¹H NMR (400 MHz, CDCl₃). MS (ES+) m/z mass calcd for C₂₄H₂₃F₃N₂O₃S 476, found 477 (M+1, 100%), MS (ES−) found 475 (M−1, 100%).

### Step B

### (S)-{3-[3-(2-Pyrimidin-2-yl-4-trifluoromethyl-phenoxy)-butylsulfanyl]-phenyl}-acetic acid

The procedure from Example 291, Step E is utilized with (S)-{3-[3-(2-pyrimidin-2-yl-4-trifluoromethyl-phenoxy)-butylsulfanyl]-phenyl}-acetic acid methyl ester to afford 0.1 g (91%) of product. ¹H NMR (400 MHz, CDCl₃). MS (ES+) m/z mass calcd for C₂₃H₂₁F₃N₂O₃S 462, found 463 (M+1, 100%), MS (ES−) found 461 (M−1, 100%).

### EXAMPLE 337

### (S)-(3-{3-[2-(4-Fluoro-phenoxy)-4-trifluoromethyl-phenoxy]-butylsulfanyl}-phenyl)-acetic acid

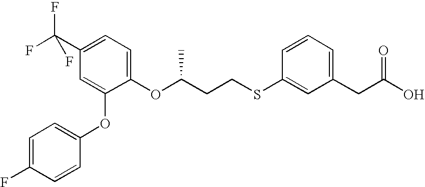

### Step A

### 2-(4-Fluoro-phenoxy)-4-trifluoromethyl-benzaldehyde

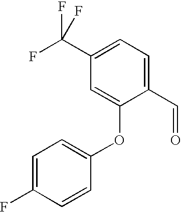

The procedure from Example 293, Step A is utilized with 2-fluoro-4-trifluoromethyl-benzaldehyde and 4-fluorophenol to afford 1.46 g (99%) of product. ¹H NMR (400 MHz, CDCl₃), MS (ES−) m/z mass calcd for C₁₄H₈F₄O₂ 284, found 343 (M+CH₃COO⁻, 80%).

### Step B

### 2-(4-Fluoro-phenoxy)-4-trifluoromethyl-phenol

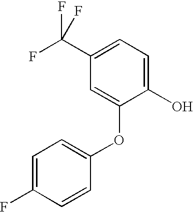

The procedure from Example 293, Step B is utilized with 2-(4-fluoro-phenoxy)-4-trifluoromethyl-benzaldehyde to afford 0.839 g (60%) of product. ¹H NMR (400 MHz, CDCl₃). MS (ES−) m/z mass calcd for C₁₃H₈F₄O₂ 272, found 271 (M−1, 100%).

### Step C

### (S)-(3-{3-[2-(4-Fluoro-phenoxy)-4-trifluoromethyl-phenoxy]-butylsulfanyl}-phenyl)-acetic acid methyl ester

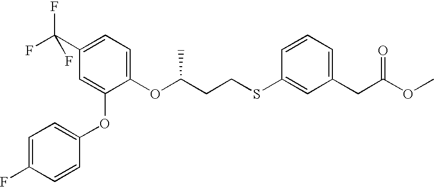

The procedure from Example 290, Step A is utilized with (R)-[3-(3-methanesulfonyloxy-butylsulfanyl)-phenyl]-acetic acid methyl ester and 2-(4-fluoro-phenoxy)₄-trifluoromethyl-phenol to afford 0.114 g (75%) of product. ¹H NMR (400 MHz, CDCl₃). MS (ES+) m/z mass calcd for C₂₆H₂₄F₄O₄S 508, found 526 (M+NH₄, 100%), MS (ES−) found 507 (M−1, 100%).

### Step D

### (S)-(3-{3-[2-(4-Fluoro-phenoxy)-4-trifluoromethyl-phenoxy]-butylsulfanyl}-phenyl)-acetic acid

The procedure from Example 291, Step E is utilized with (S)-(3-{3-[2-(4-fluoro-phenoxy)-4-trifluoromethyl-phenoxy]-butylsulfanyl}-phenyl)-acetic acid methyl ester to afford 0.091 g (82%) of product. ¹H NMR (400 MHz, CDCl₃). MS (ES+) m/z mass calcd for C₂₅H₂₂F₄O₄S 494, found 512 (M+NH₄, 100%), MS (ES−) found 493 (M−1, 100%).

### EXAMPLE 338

### (S)-(3-{3-[2-(2,4-Difluoro-phenoxy)-4-trifluoromethyl-phenoxy]-butylsulfanyl}-phenyl)-acetic acid

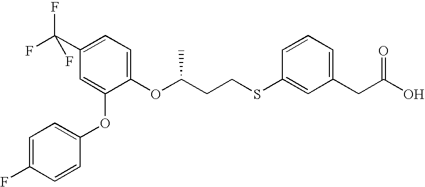

### Step A

### 2-(2,4-Difluoro-phenoxy)-4-trifluoromethyl-benzaldehyde

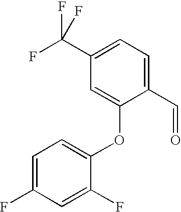

The procedure from Example 293, Step A is utilized with 2-fluoro-4-trifluoromethyl-benzaldehyde and 2,4-Difluorophenol to afford 1.25 g (80%) of product. ¹H NMR (400 MHz, CDCl₃), MS (ES−) m/z mass calcd for C₁₄H₇F₅O₂ 302, found 361 (M+CH₃COO⁻, 80%).

### Step B

### 2-(2,4-Difluoro-phenoxy)-4-trifluoromethyl-phenol

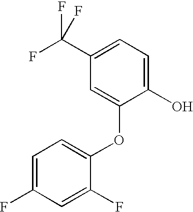

The procedure from Example 293, Step B is utilized with 2-(2,4-difluoro-phenoxy)-4-trifluoromethyl-benzaldehyde to afford 0.696 g (58%) of product. ¹H NMR (400 MHz, CDCl₃). MS (ES−) m/z mass calcd for C₁₃H₇F₅O₂ 290, found 289 (M−1, 100%).

### Step C

### (S)-(3-{3-[2-(2,4-Difluoro-phenoxy)-4-trifluoromethyl-phenoxy]-butylsulfanyl}-phenyl)-acetic acid methyl ester

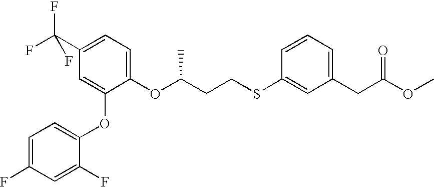

The procedure from Example 290, Step A is utilized with (R)-[3-(3-methanesulfonyloxy-butylsulfanyl)-phenyl]-acetic acid methyl ester and 2-(2,4-difluoro-phenoxy)-4-trifluoromethyl-phenol to afford 0.047 g (30%) of product. ¹H NMR (400 MHz, CDCl₃). MS (ES+) m/z mass calcd for C₂₆H₂₃F₅O₄S 526, found 544 (M+NH₄, 100%), MS (ES−) found 525 (M−1, 100%).

### Step D

### (S)-(3-{3-[2-(2,4-Difluoro-phenoxy)-4-trifluoromethyl-phenoxy]-butylsulfanyl}-phenyl)-acetic acid

The procedure from Example 291, Step E is utilized with (S)-(3-{3-[2-(2,4-difluoro-phenoxy)-4-trifluoromethyl-phenoxy]-butylsulfanyl}-phenyl)-acetic acid methyl ester to afford 0.018 g (39%) of product. ¹H NMR (400 MHz, CDCl₃). MS (ES+) m/z mass calcd for C₂₅H₂₁F₅O₄S 512, found 530 (M+NH₄, 100%), MS (ES−) found 511 (M−1, 100%).

### EXAMPLE 339

### (S)-3-{2-Methyl-4-[3-(2-pyrimidin-2-yl-4-trifluoromethyl-phenoxy)-butylsulfanyl]-phenyl}-propionic acid

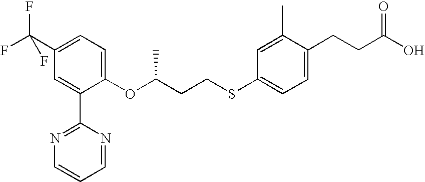

The procedure from Example 284, Step C is utilized with (R)-3-[4-(3-methanesulfonyloxy-butylsulfanyl)-2-methyl-phenyl]-propionic acid ethyl ester and 2-pyrimidin-2-yl-4-trifluoromethyl-phenol to afford 0.076 g (56%) of product. ¹H NMR (400 MHz, CDCl₃). MS (ES+) m/z mass calcd for C₂₅H₂₅F₃N₂O₃S 490, found 491 (M+1, 100%), MS (ES−) found 489 (M−1, 100%).

### EXAMPLE 340

### (S)-3-(4-{3-[2-(4-Fluoro-phenoxy)-4-trifluoromethyl-phenoxy]-butylsulfanyl}-2-methyl-phenyl)-propionic acid

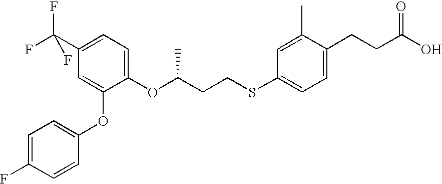

The procedure from Example 284, Step C is utilized with (R)-3-[4-(3-methanesulfonyloxy-butylsulfanyl)-2-methyl-phenyl]-propionic acid ethyl ester and 2-(4-fluoro-phenoxy)-4-trifluoromethyl-phenol to afford 0.078 g (54%) of product. ¹H NMR (400 MHz, CDCl₃). MS (ES+) m/z mass calcd for C₂₇H₂₆F₄O₄S 522, found 540 (M+NH₄, 100%), MS (ES−) found 521 (M−1, 100%).

### EXAMPLE 341

### (S)-3-(4-{3-[2-(2,4-Difluoro-phenoxy)-4-trifluoromethyl-phenoxy]-butylsulfanyl}-2-methyl-phenyl)-propionic acid

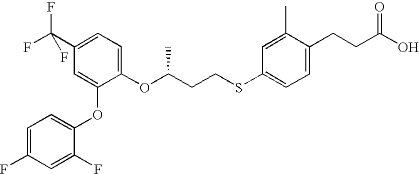

The procedure from Example 284, Step C is utilized with (R)-3-[4-(3-methanesulfonyloxy-butylsulfanyl)-2-methyl-phenyl]-propionic acid ethyl ester and 2-(2,4-difluoro-phenoxy)-4-trifluoromethyl-phenol to afford 0.045 g (30%) of product. ¹H NMR (400 MHz, CDCl₃). MS (ES+) m/z mass calcd for C₂₇H₂₅F₅O₄S 540, found 558 (M+NH4, 100%), MS (ES−) found 539 (M−1, 100%).

### EXAMPLE 342

### (S)-3-{2-Ethyl-4-[3-(2-pyrimidin-2-yl-4-trifluoromethyl-phenoxy)-butylsulfanyl]-phenyl}-propionic acid

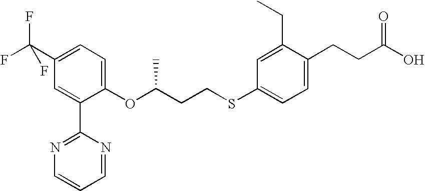

The procedure from Example 284, Step C is utilized with (R)-3-[4-(3-methanesulfonyloxy-butylsulfanyl)-2-ethyl-phenyl]-propionic acid ethyl ester and 2-pyrimidin-2-yl-4-trifluoromethyl-phenol to afford 0.004 g (3%) of product. ¹H NMR (400 MHz, CDCl₃). MS (ES+) m/z mass calcd for C₂₆H₂₇F₃N₂O₃S 504, found 505 (M+1, 100%), MS (ES−) found 503 (M−1, 100%).

### EXAMPLE 343

### (S)-3-{2-Ethyl-4-[3-(4-ethyl-2-pyridin-2-yl-phenoxy)-butylsulfanyl]-phenyl}-propionic acid

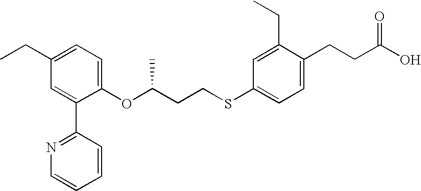

The procedure from Example 284, Step C is utilized with (R)-3-[4-(3-methanesulfonyloxy-butylsulfanyl)-2-ethyl-phenyl]-propionic acid ethyl ester and 4-ethyl-2-pyridin-2-yl-phenol to afford 0.014 g (12%) of product. ¹H NMR (400 MHz, CDCl₃). MS (ES+) m/z mass calcd for C₂₈H₃₃NO₃S 463, found 464 (M+1, 100%), MS (ES−) found 462 (M−1, 100%).

### EXAMPLE 344

### (S)-3-(4-{3-[2-(4-Fluoro-phenoxy)-4-trifluoromethyl-phenoxy]-butylsulfanyl}-2-ethyl-phenyl)-propionic acid

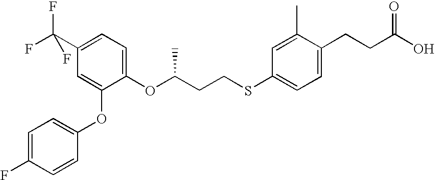

The procedure from Example 284, Step C is utilized with (R)-3-[4-(3-methanesulfonyloxy-butylsulfanyl)-2-ethyl-phenyl]-propionic acid ethyl ester and 2-(4-fluoro-phenoxy)-4-trifluoromethyl-phenol to afford 0.09 g (65%) of product. ¹H NMR (400 MHz, CDCl₃). MS (ES+) m/z mass calcd for C₂₈H₂₈F₄O₄S 536, found 554 (M+NH₄, 100%), MS (ES−) found 535 (M−1, 100%).

### EXAMPLE 345

### (S)-3-(4-{3-[2-(2,4-Difluoro-phenoxy)-4-trifluoromethyl-phenoxy]-butylsulfanyl}-2-ethyl-phenyl)-propionic acid

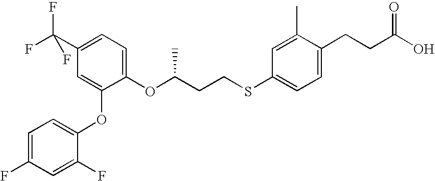

The procedure from Example 284, Step C is utilized with (R)-3-[4-(3-methanesulfonyloxy-butylsulfanyl)-2-ethyl-phenyl]-propionic acid ethyl ester and 2-(2,4-difluoro-phenoxy)-4-trifluoromethyl-phenol to afford 0.085 g (59%) of product. ¹H NMR (400 MHz, CDCl₃). MS (ES+) m/z mass calcd for C₂₈H₂₇F₅O₄S 554, found 572 (M+NH₄, 100%), MS (ES−) found 553 (M−1, 100%).

### EXAMPLE 346

### (S)-{3-[3-(4-Ethyl-2-pyridin-2-yl-phenoxy)-butylsulfanyl]-phenyl}-acetic acid

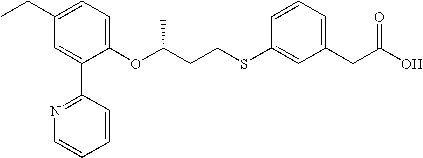

The procedure from Example 284, Step C is utilized with (R)-[3-(3-methanesulfonyloxy-butylsulfanyl)-phenyl]-acetic acid methyl ester and 4-ethyl-2-pyridin-2-yl-phenol to afford 0.005 g (3%) of product. ¹H NMR (400 MHz, CDCl₃). MS (ES+) m/z mass calcd for C₂₅H₂₇NO₃S 421, found 422 (M+1, 100%), MS (ES−) found 420 (M−1, 100%).

### EXAMPLE 347

### (S)-3-{2-Ethyl-4-[3-(4-ethyl-2-o-tolyloxy-phenoxy)-butoxy]-phenyl}-propionic acid

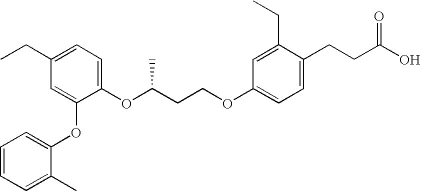

### Step A

### 4-Ethyl-1-methoxy-2-o-tolyloxy-benzene

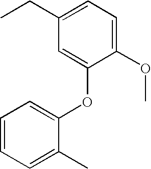

The procedure from Example 290, Step B is utilized with 2-bromo-4-ethyl-1-methoxy-benzene and o-cresol to afford 1.13 g (67%) of product. ¹H NMR (400 MHz, CDCl₃), MS (ES+) m/z mass calcd for C₁₆H₁₈O₂ 242, found 260 (M+NH₄, 100%).

### Step B

### 4-Ethyl-2-o-tolyloxy-phenol

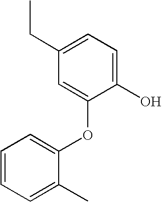

A solution of 4-ethyl-1-methoxy-2-o-tolyloxy-benzene (1.13 g, 4.66 mmol) in dichloromethane (10 mL) is cooled to −78° C. The solution is then treated with 1M boron tribromide in dichloromethane (23 mL, 23 mmol). The reaction is warmed to room temperature and stirred for 2 hr. The reaction is then quenched with water followed by 1N aqueous HCl to pH=7. The aqueous is extracted with dichloromethane. The organic is washed with brine, dried over sodium sulfate, filtered, and the solvent is removed. The crude is purified by silica gel column chromatography using 4:1 hexanes:ethyl acetate to elute the pure product. The solvent is removed to afford 0.567 g (53%) of product. ¹H NMR (400 MHz, CDCl₃). MS (ES−) m/z mass calcd for C₁₅H₁₆O₂ 228, found 227 (M−1, 100%).

### Step C

### (S)-3-{2-Ethyl-4-[3-(4-ethyl-2-o-tolyloxy-phenoxy)-butoxy]-phenyl}-propionic acid ethyl ester

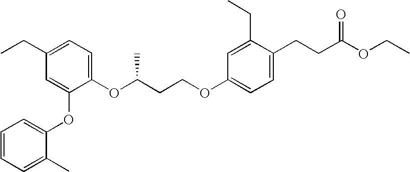

The procedure from Example 290, Step A is utilized with (R)-3-[2-ethyl-4-(3-methanesulfonyloxy-butoxy)-phenyl]-propionic acid ethyl ester and 4-ethyl-2-o-tolyloxy-phenol to afford 0.322 g (48%) of product. ¹H NMR (400 MHz, CDCl₃). MS (ES+) m/z mass calcd for C₃₂H₄₀O₅ 504, found 522 (+NH₄, 100%).

### Step D

### (S)-3-{2-Ethyl-4-[3-(4-ethyl-2-o-tolyloxy-phenoxy)-butoxy]-phenyl}-propionic acid

The procedure from Example 294, Step B is utilized with (S)-3-{2-ethyl-4-[3-(4-ethyl-2-o-tolyloxy-phenoxy)-butoxy]-phenyl}-propionic acid ethyl ester to afford 0.23 g (76%) of product. ¹H NMR (400 MHz, CDCl₃). MS (ES+) m/z mass calcd for C₃₀H₃₆O₅ 476, found 494 (M+NH₄, 20%), MS (ES−) found 475 (M−1, 100%).

### EXAMPLE 348

### (S)-3-(4-{3-[4-Chloro-2-(4-fluoro-phenoxy)-phenoxy]-butylsulfanyl}-2-ethyl-phenyl)-propionic acid

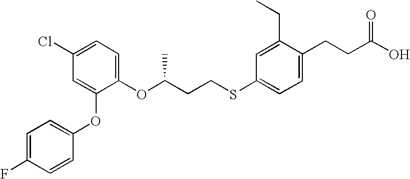

The procedure from Example 284, Step C is utilized with (R)-3-[2-ethyl-4-(3-methanesulfonyloxy-butylsulfanyl)-phenyl]-propionic acid ethyl ester and 4-chloro-2-(4-fluoro-phenoxy)-phenol to afford 0.106 g (81%) of product. ¹H NMR (400 MHz, CDCl₃). MS (ES+) m/z mass calcd for C₂₇H₂₈ClFO₄S 502, found 520 (M+NH₄, 100%), MS (ES−) found 501 (M−1, 100%).

### EXAMPLE 349

### (S)-(3-{3-[4-Chloro-2-(4-fluoro-phenoxy)-phenoxy]-butylsulfanyl}-phenyl)-acetic acid

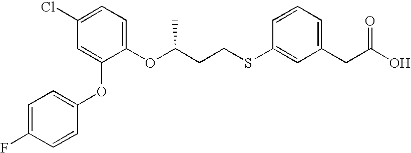

The procedure from Example 284, Step C is utilized with (R)-[3-(3-methanesulfonyloxy-butylsulfanyl)-phenyl]-acetic acid methyl ester and 4-chloro-2-(4-fluoro-phenoxy)-phenol to afford 0.073 g (53%) of product. ¹H NMR (400 MHz, CDCl₃). MS (ES+) m/z mass calcd for C₂₄H₂₂ClFO₄S 460, found 478 (M+NH₄, 100%), MS (ES−) found 459 (M−1, 100%).

### EXAMPLE 350

### (S)-3-{2-Ethyl-4-[3-(2-o-tolyloxy-4-trifluoromethoxy-phenoxy)-butoxy]-phenyl}-propionic acid

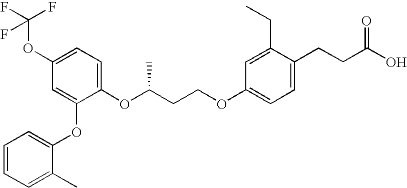

### Step A

### 1-Methoxy-2-o-tolyloxy-4-trifluoromethoxy-benzene

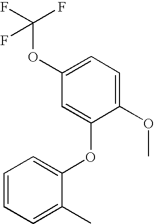

The procedure from Example 290, Step B is utilized with 2-bromo-1-methoxy-4-trifluoromethoxy-benzene and o-cresol to afford 5.8 g (59%) of product. ¹H NMR (400 MHz, CDCl₃), MS (ES+) m/z mass calcd for C₁₅H₁₃F₃O₃ 298, found 316 (M+NH₄, 100%).

### Step B

### 2-o-Tolyloxy-4-trifluoromethoxy-phenol

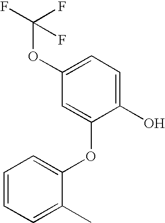

The procedure from Example 314, Step B is utilized with 1-methoxy-2-o-tolyloxy-4-trifluoromethoxy-benzene to afford 5.11 g (93%) of product. ¹H NMR (400 MHz, CDCl₃). MS (ES−) m/z mass calcd for C₁₄H₁₁F₃O₃ 284, found 283 (M−1, 100%).

### Step C

### (S)-3-{2-Ethyl-4-[3-(2-o-tolyloxy-4-trifluoromethoxy-phenoxy)-butoxy]-phenyl}-propionic acid ethyl ester

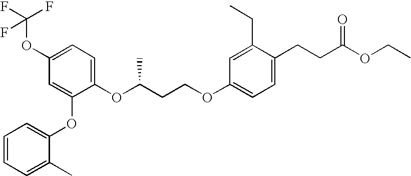

The procedure from Example 290, Step A is utilized with (R)-3-[2-ethyl-4-(3-methanesulfonyloxy-butoxy)-phenyl]-propionic acid ethyl ester and 2-o-tolyloxy-4-trifluoromethoxy-phenol to afford 0.176 g (78%) of product. ¹H NMR (400 MHz, CDCl₃). MS (ES+) m/z mass calcd for C₃₁H₃₅F₃O₆ 560, found 578 (M+NH₄, 100%).

### Step D

### (S)-3-{2-Ethyl-4-[3-(2-o-tolyloxy-4-trifluoromethoxy-phenoxy)-butoxy]-phenyl}-propionic acid

The procedure from Example 294, Step B is utilized with (S)-3-{2-ethyl-4-[3-(2-o-tolyloxy-4-trifluoromethoxy-phenoxy)-butoxy]-phenyl}-propionic acid ethyl ester to afford 0.152 g (91%) of product. ¹H NMR (400 MHz, CDCl₃). MS (ES+) m/z mass calcd for C₂₉H₃₁F₃O₆ 532, found 550 (M+NH₄, 20%), MS (ES−) found 531 (M−1, 100%).

### EXAMPLE 351

### (S)-3-{2-Ethyl-4-[3-(2-o-tolyloxy-4-trifluoromethyl-phenoxy)-butoxy]-phenyl}-propionic acid

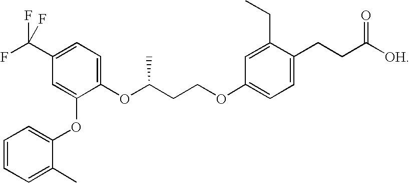

The procedure from Example 290, Step B is utilized with 3-{4-[3-(2-bromo-4-trifluoromethyl-phenoxy)-butoxy]-2-ethyl-phenyl}-propionic acid ethyl ester and o-cresol to afford 0.007 g (5%) of product. ¹H NMR (400 MHz, CDCl₃). MS (ES+) m/z mass calcd for C₂₉H₃₁F₃O₅ 516, found 534 (M+NH₄, 100%), MS (ES−) found 515 (M−1,100%).

### EXAMPLE 352

### (S)-(4-{3-[2-(2-Fluoro-phenoxy)-4-trifluoromethyl-phenoxy]-butoxy}-3-methyl-phenyl)-acetic acid

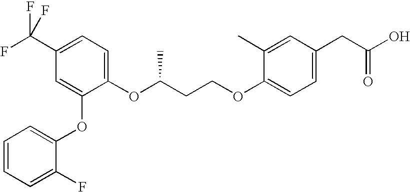

The procedure from Example 284, Step C is utilized with (R)-[4-(3-methanesulfonyloxy-butoxy)-3-methyl-phenyl]-acetic acid methyl ester and 2-(2-fluoro-phenoxy)-4-trifluoromethyl-phenol to afford 0.089 g (60%) of product. ¹H NMR (400 MHz, CDCl₃). MS (ES+) m/z mass calcd for C₂₆H₂₄F₄O₅ 492, found 510 (M+NH₄, 100%), MS (ES−) found 491 (M−1, 100%).

### EXAMPLE 353

### (S)-(4-{3-[2-(2-Fluoro-phenoxy)-4-trifluoromethyl-phenoxy]-butoxy}-2-methyl-phenylsulfanyl)-acetic acid

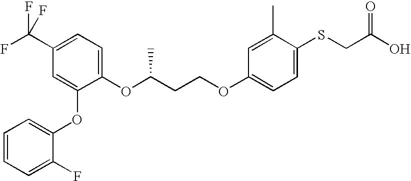

The procedure from Example 284, Step C is utilized with (R)-[4-(3-methanesulfonyloxy-butoxy)-2-methyl-phenylsulfanyl]-acetic acid ethyl ester and 2-(2-fluoro-phenoxy)-4-trifluoromethyl-phenol to afford 0.079 g (57%) of product. ¹H NMR (400 MHz, CDCl₃). MS (ES+) m/z mass calcd for C₂₆H₂₄F₄O₅S 524, found 542 (M+NH₄, 100%), MS (ES−) found 523 (M−1, 100%).

### EXAMPLE 354

### (S)-(4-{3-[2-(2-Fluoro-phenoxy)-4-trifluoromethyl-phenoxy]-butylsulfanyl}-2-methyl-phenoxy)-acetic acid

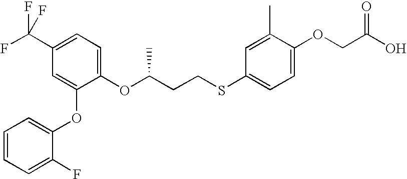

The procedure from Example 284, Step C is utilized with (R)-[4-(3-methanesulfonyloxy-butylsulfanyl)-2-methyl-phenoxy]-acetic acid ethyl ester and 2-(2-fluoro-phenoxy)-4-trifluoromethyl-phenol to afford 0.096 g (69%) of product. ¹H NMR (400 MHz, CDCl₃). MS (ES+) m/z mass calcd for C₂₆H₂₄F₄O₅S 524, found 542 (M+NH₄, 100%), MS (ES−) found 523 (M−1, 100%).

### EXAMPLE 355

### (S)-3-{4-[3-(4-Chloro-2-phenoxy-phenoxy)-butylsulfanyl]-2-ethyl-phenyl}-propionic acid

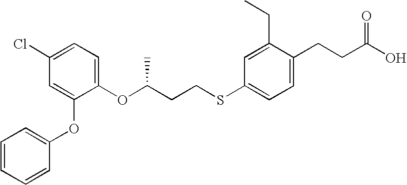

The procedure from Example 284, Step C is utilized with (R)-3-[2-ethyl-4-(3-methanesulfonyloxy-butylsulfanyl)-phenyl]-propionic acid ethyl ester and 4-chloro-2-phenoxy-phenol to afford 0.068 g (54%) of product. ¹H NMR (400 MHz, CDCl₃). MS (ES+) m/z mass calcd for C₂₇H₂₉ClO₄S 484, found 502 (M+NH₄, 100%), MS (ES−) found 483 (M−1, 100%).

### EXAMPLE 356

### (S)-3-{2-Ethyl-4-[3-(2-o-tolyloxy-4-trifluoromethyl-phenoxy)-butylsulfanyl]-phenyl}-propionic acid

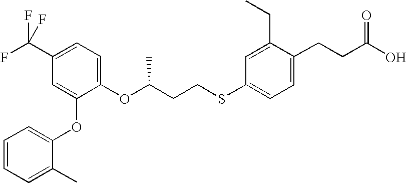

### Step A

### 2-o-Tolyloxy-4-trifluoromethyl-benzaldehyde

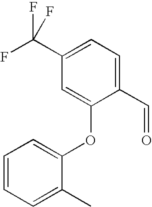

The procedure from Example 293, Step A is utilized with 2-fluoro-4-trifluoromethyl-benzaldehyde and o-cresol to afford 3.7 g (84%) of product. ¹H NMR (400 MHz, CDCl₃), MS (ES−) m/z mass calcd for C₁₅H₁₁F₃O₂ 280, found 339 (M+CH₃COO⁻, 100%).

### Step B

### 2-o-Tolyloxy-4-trifluoromethyl-phenol

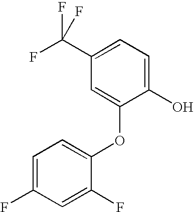

The procedure from Example 293, Step B is utilized with 2-o-tolyloxy-4-trifluoromethyl-benzaldehyde to afford 2.08 g (59%) of product. ¹H NMR (400 MHz, CDCl₃). MS (ES−) m/z mass calcd for C₁₄H₁₁F₃O₂ 268, found 267 (M−1, 100%).

### Step C

### (S)-3-{2-Ethyl-4-[3-(2-o-tolyloxy-4-trifluoromethyl-phenoxy)-butylsulfanyl]-phenyl}-propionic acid

The procedure from Example 284, Step C is utilized with (R)-3-[2-ethyl-4-(3-methanesulfonyloxy-butylsulfanyl)-phenyl]-propionic acid ethyl ester and 2-o-tolyloxy-4-trifluoromethyl-phenol to afford 0.084 g (61%) of product. ¹H NMR (400 MHz, CDCl₃). MS (ES+) m/z mass calcd for C₂₉H₃₁F₃O₄S 532, found 550 (M+NH₄, 100%), MS (ES−) found 531 (M−1, 100%).

### EXAMPLE 357

### (S)-3-(2-Ethyl-4-{3-[2-(2-fluoro-phenoxy)-4-trifluoromethyl-phenoxy]-butylsulfanyl}-phenyl)-propionic acid

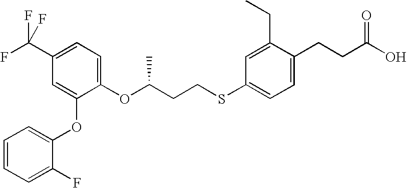

The procedure from Example 284, Step C is utilized with (R)-3-[2-ethyl-4-methanesulfonyloxy-butylsulfanyl)-phenyl]-propionic acid ethyl ester and 2-(2-fluoro-enoxy)-4-trifluoromethyl-phenol to afford 0.092 g (67%) of product. ¹H NMR (400 Hz, CDCl₃). MS (ES+) m/z mass calcd for C₂₈H₂₈F₄O₄S 536, found 554 (M+NH₄, 0%), MS (ES−) found 535 (M−1, 100%).

### EXAMPLE 358

### )-3-(2-Ethyl-4-{3-[2-(2-fluoro-phenoxy)-4-trifluoromethyl-phenoxy]-butoxy}-phenyl)-propionic acid

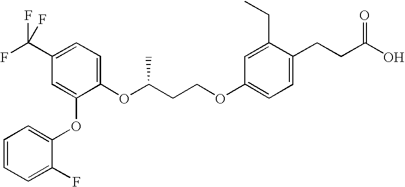

The procedure from Example 284, Step C is utilized with (R)-3-[2-ethyl- [symbol] ethanesulfonyloxy-butoxy)-phenyl]-propionic acid ethyl ester and 2-(2-fluoro- [symbol] )-4-trifluoromethyl-phenol to afford 0.065 g (46%) of product. ¹H NMR (400 [symbol] Cl₃). MS (ES+) m/z mass calcd for C₂₈H₂₈F₄O₅ 520, found 538 (M+NH₄, [symbol] IS (ES−) found 519 (M−1, 100%).

1. A compound having a formula I, 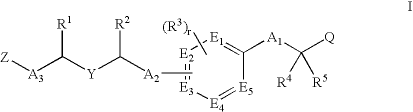
or a pharmaceutically acceptable salt, solvate, hydrate or stereoisomer thereof, wherein:
A₁ is: a bond, CH₂, O or S, and wherein A₁ and R⁴ or A₁ and R⁵ together being a 3- to 6-membered carbocyclyl when A₁ is a carbon;
A₂ and A₃ are independently: CH₂, O or S;
E₁, E₂, E₃, E₄ and E₅ are each CH or substituted carbon bearing A₂ and R³; or at least one of E₁, E₂, E₃, E₄ and E₅ is nitrogen and each of others being CH or substituted carbon bearing A₂ and R³;
Q is: —C(O)OR⁶, or R^{6A};
Y is: a bond, C₁-C₆ alkyl or C₃-C₆ cycloalkyl;
Z is: a) aryl;
b) a 5- to 10-membered heteroaryl wherein the heteroaryl containing at least one heteroatom selected from N, O or S,
c) bi-aryl, wherein biaryl being defined as aryl substituted with another aryl or aryl substituted with heteroaryl, or
d) bi-heteroaryl, wherein bi-heteroaryl being defined as heteroaryl substituted with another heteroaryl, or heteroaryl substituted with aryl, and wherein aryl, heteroaryl, bi-aryl and bi-heteroaryl being optionally substituted with one or more groups independently selected from R⁷;
n is: 1,2,3,4, 5 or 6
p is: 1 or 2;
r is: 1,2,3, or 4;
R¹ and R² are each independently:
hydrogen,
haloalkyl,
C₁-C₆ alkyl,
(CH₂)ₙC₃-C₈ cycloalkyl, or
R¹ and R² form a 4- to 8-membered nonaromatic carbocyclic ring; and
wherein at least one of R¹ and R² is alkyl or cycloalkyl, and;
R³ is: hydrogen,
nitro,
cyano,
hydroxyl,
halo,
haloalkyl,
haloalkyloxy,
aryloxy,
C₁-C₆ alkyl,
C₁-C₆ alkoxy, or
C₃-C₈ cycloalkyl;
R⁴ and R⁵ are each independently: hydrogen or C₁-C₆ alkyl;
R⁶ is: hydrogen, C₁-C₆ alkyl or aminoalkyl;
R^{6A} is: carboxamide, sulfonamide, acylsulfonamide, tetrazole, 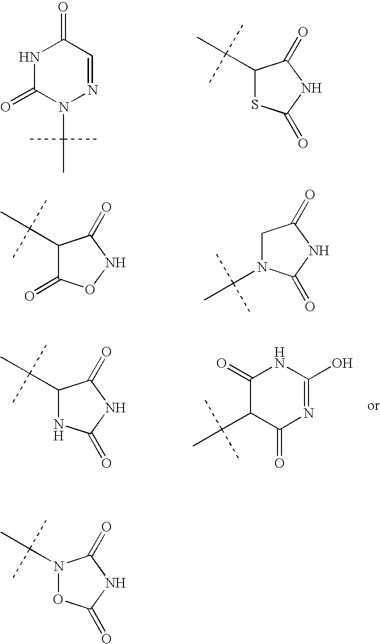
R⁷ is: hydrogen,
oxo,
nitro,
cyano,
hydroxyl,
halo,
haloalkyl,
haloalkyloxy,
aryloxy,
arylalkyl,
aminoalkyl,
C₁-C₆ alkyl,
C₁-C₆ alkoxy,
(CH₂)ₙC₃-C₈ cycloalkyl,
C(O)R⁹,
C(O)OR⁹,
C(═NOR⁸)R⁹,
CR⁸(OH)R⁹,
C[═C(R⁸)₂]R⁹,
OR⁹,
SR⁹ or
S(O)ₚR⁹;
R⁸ is: hydrogen or C₁-C₆ alkyl; and
R⁹ is: hydrogen,
C₁-C₆ alkyl,
C₃-C₈ cycloalkyl,
aryl,
heteroaryl or
heterocyclyl,
wherein alkyl, cycloalkyl, aryl, heteroaryl or heterocyclyl being optionally substituted with one or more substituents selected from the group consisting of:
hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

2. The compound of claim 1, wherein the compound is represented by a compound of formula II, 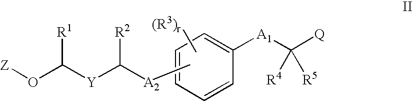
or a pharmaceutically acceptable salt, solvate, hydrate or stereoisomer thereof, wherein:
A₁ is: a bond, CH₂, O or S, and wherein A₁ and R⁴ or A₁ and R⁵ together being a 3- to 6-membered carbocyclyl when A₁ is a carbon;
A₂ is: O or S or CH₂;
Q is: —C(O)OR⁶, or R^{6A};
Y is: a bond, C₁-C₆ alkyl or C₃-C₆ cycloalkyl;
Z is: a) aryl;
b) a 5- to 10-membered heteroaryl wherein the heteroaryl containing at least one heteroatom selected from N, O or S,
c) bi-aryl, wherein biaryl being defined as aryl substituted with another aryl or aryl substituted with heteroaryl, or
d) bi-heteroaryl, wherein bi-heteroaryl being defined as heteroaryl substituted with another heteroaryl, or heteroaryl substituted with aryl, and wherein aryl, heteroaryl, bi-aryl and bi-heteroaryl being optionally substituted with one or more groups independently selected from R⁷;
n is: 1, 2, 3, 4, 5 or 6
p is: 1 or 2;
r is: 1, 2, 3, or 4;
R¹ and R² are each independently:
hydrogen,
haloalkyl,
C₁-C₆ alkyl,
(CH₂)ₙC₃-C₈ cycloalkyl, or
R¹ and R² form a 4- to 8-membered nonaromatic carbocyclic ring; and
wherein at least one of R¹ and R² is alkyl or cycloalkyl, and;
R³ is: hydrogen,
nitro,
cyano,
hydroxyl,
halo,
haloalkyl,
haloalkyloxy,
aryloxy,
C₁-C₆ alkyl,
C₁-C₆ alkoxy, or
C₃-C₈ cycloalkyl;
R⁴ and R⁵ are each independently: hydrogen or C₁-C₆ alkyl;
R⁶ is: hydrogen, C₁-C₆ alkyl or aminoalkyl;
R^{6A} is: carboxamide, sulfonamide, acylsulfonamide, tetrazole, 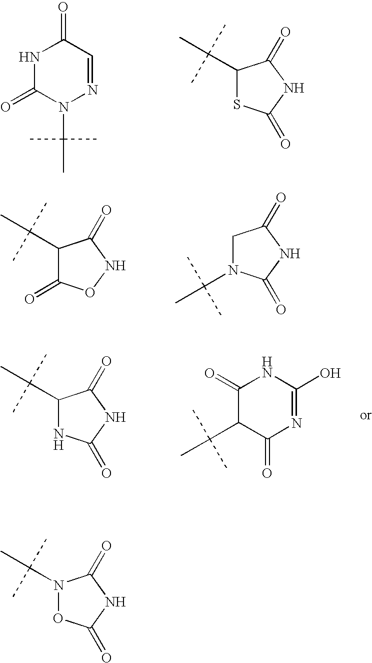
R⁷ is: hydrogen,
oxo,
nitro,
cyano,
hydroxyl,
halo,
haloalkyl,
haloalkyloxy,
aryloxy,
arylalkyl,
aminoalkyl,
C₁-C₆ alkyl,
C₁-C₆ alkoxy,
(CH₂)ₙC₃-C₈ cycloalkyl,
C(O)R⁹,
C(O)OR⁹,
C(═NOR⁸)R⁹,
CR⁸(OH)R⁹,
C[═C(R⁸)₂]R⁹,
R⁹,
SR⁹ or
S(O)ₚR⁹;
R⁸ is: hydrogen or C₁-C₆ alkyl; and
R⁹ is: hydrogen,
C₁-C₆ alkyl,
C₃-C₈ cycloalkyl,
aryl,
heteroaryl or
heterocyclyl,
wherein alkyl, cycloalkyl, aryl, heteroaryl or heterocyclyl being optionally substituted with one or more substituents selected from the group consisting of:
hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

3. The compound of claim 2, wherein Z is optionally substituted phenyl or naphthyl, furanyl, imidazolyl, indolyl, oxazolyl, isoxazolyl, pyridyl, pyrrolyl, thiazolyl, thiophenyl, benzofuranyl, benzothiophenyl, benzoisoxazolyl, quinolinyl, isoquinolinyl or a structural formula selected from following: 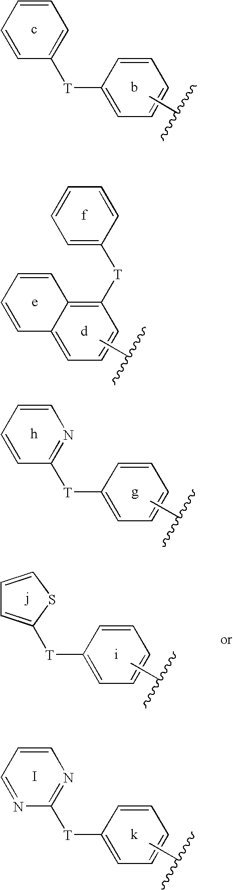
wherein T is:
a bond, —(CH₂)_{q}O—, —O(CH₂)_{q}—, —C(O)(CH₂)_{q}—, —(CH₂)_{q}C(O)—, —(CH₂)_{q}S—, —S(CH₂)_{q}—, S[O]ₚ, —(C₁-C₃ alkyl)-, —(CH₂)_{q}C(═CH₂)—, —C(═CH₂)(CH₂)_{q}—, —(CH₂)_{q}C(═NOH)—, —C(═NOH)(CH₂)_{q}—, —(CH₂)_{q}C(═NOCH₃)—, —C(═NOCH₃)(CH₂)_{q}—, —CH(OH)(CH₂)_{q}—, or —(CH₂)_{q}CH(OH)—,
q is: 0, 1, 2 or 3; and
rings b to l are each optionally substituted with one or more groups independently selected from the group consisting of:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

4. The compound of claim 2, wherein the compound having a structural formula III,

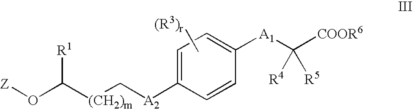

5. The compound of claim 2, wherein the compound is represented by structural formula IV, 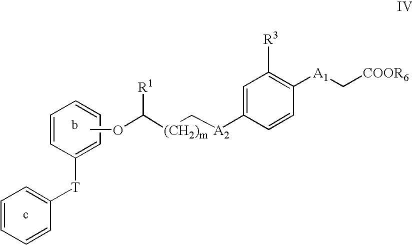
or a pharmaceutically acceptable salt, solvate, hydrate or stereoisomer thereof, wherein:
A₁ and A₂ are respectively:
O and O,
CH₂ and O,
CH₂ and S,
O and S or
S and O;
m is: 1 or 2;
R¹ is: C₁-C₃alkyl;
R³ is: hydrogen, halo or C₁-C₆ alkyl;
R⁶ and R⁹ are each independently: hydrogen or C₁-C₆ alkyl;
T is: a bond, —O—, —C(O)—, —S(O)—S(O)₂—, —C(═CH₂)—, —C(═NOH)— or —CH(OH)—; and
rings b and c are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

6. The compound of claim 5, wherein the compound is represented by structural formula V, 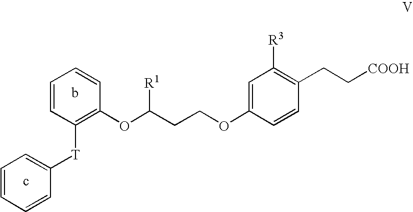
or a pharmaceutically acceptable salt, solvate, hydrate or stereoisomer thereof, wherein:
T is: a bond, —O— or —C(O)—;
R¹ is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, methyl, ethyl, isopropyl, N(CH₃)₂, S(O)₂CH₃, methoxy and cyclopropyl.

7. The compound of claim 6, wherein the compound is represented by a structural formula VI, 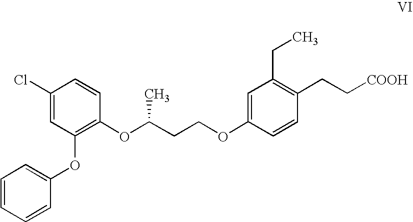
or a pharmaceutically acceptable salt, solvate or hydrate thereof.

8. The compound of claim 2, wherein the compound having a structural formula VII, 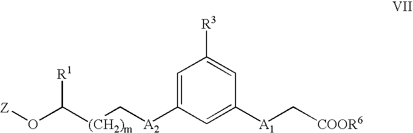
Z is: 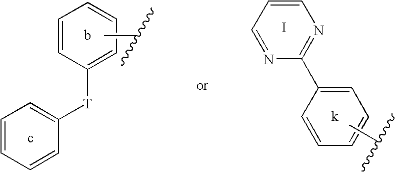
A₁ and A₂ are respectively: bond and S; bond and O; CH₂ and S; or CH₂ and O;
m is: 1 or 2;
R¹ is: C₁-C₃alkyl;
R³ is: hydrogen, halo or C₁-C₆ alkyl;
R⁶ and R⁹ are each independently: hydrogen or C₁-C₆ alkyl;
T is: bond, —O—, —C(O)—, —S(O)—S(O)₂—, —C(═CH₂)—, —C(═NOH)— or —CH(OH)—; and
rings b, c, k and l are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

9. The compound of claim 8, wherein R¹ is: methyl, ethyl or cyclopropyl; R³ is: methyl or ethyl; and rings b, c k and l are each optionally substituted with one or more substituent independently selected from the group consisting of: hydrogen, Cl, Br, CF₃, OCF₃, N(CH₃)₂, S(O)₂CH₃, methyl, ethyl, isopropyl, methoxy and cyclopropyl.

10. The compound of claim 2, wherein the compound is represented by structural formula VIII, 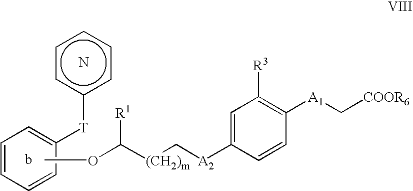
or a pharmaceutically acceptable salt, solvate, hydrate or stereoisomer thereof, wherein:
A₁ and A₂ are respectively:
O and O,
CH₂ and O,
CH₂ and S,
O and S or
S and O;
m is: 1 or 2;
R¹ is: C₁-C₃ alkyl; and
R³ is: hydrogen, halo or C₁-C₆ alkyl;
R⁶ and R⁹ are each independently: hydrogen or C₁-C₆ alkyl;
T is: a bond, —O—, —C(O)—, —S(O)—S(O)₂—, —C(═CH₂)—, —C(—NOH)— or —CH(OH)—; and
ring b is optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

11. The compound of claim 10, wherein the compound is represented by structural formula IX, 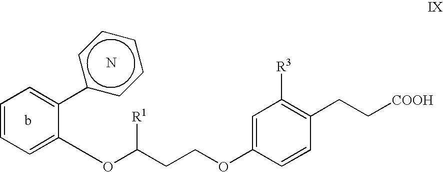
or a pharmaceutically acceptable salt, solvate, hydrate or stereoisomer thereof, wherein:
R¹ is C₁-C₃ alkyl;
R³ is: hydrogen, halo or C₁-C₄ alkyl;
ring b is optionally substituted with one or more groups independently selected from the group consisting of: hydrogen, halo, haloalkyl, haloalkyloxy and C₁-C₆ alkyl.

12. The compound of claim 11, wherein the compound is represented by structural formula X, 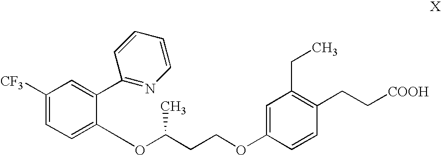
or a pharmaceutically acceptable salt, solvate or hydrate thereof.

13. The compound of claim 11, wherein the compound is represented by structural formula XI, 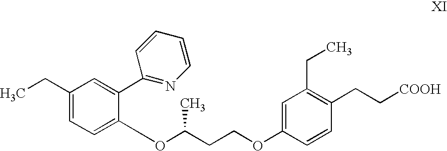
or a pharmaceutically acceptable salt, solvate or hydrate thereof.

14. The compound of claim 2, wherein the compound is represented by structural formula XII, 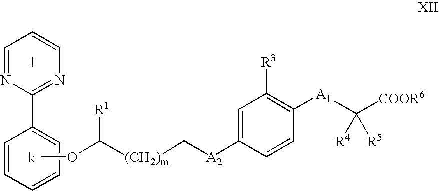
or a pharmaceutically acceptable salt, solvate, hydrate or stereoisomer thereof, wherein:
A₁ and A₂ are respectively:
and O,
CH₂ and O,
CH₂ and S,
O and S or
S and O;
m is: 1 or 2;
R¹ is: C₁-C₃ alkyl; and
R³ is: hydrogen, halo or C₁-C₆ alkyl;
R⁴, R⁵, R⁶ and R⁹ are each independently: hydrogen or C₁-C₆ alkyl;
rings k and l are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

15. (canceled)

16. The compound of claim 2, wherein the compound is represented by structural formula XIII, 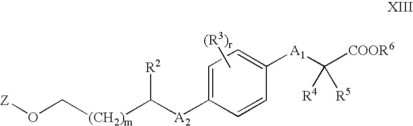
or a pharmaceutically acceptable salt, solvate, hydrate or stereoisomer thereof, wherein m is 1, 2, 3, or 4.

17. (canceled)

18. The compound of claim 16, wherein the compound is represented by structural formula XV, 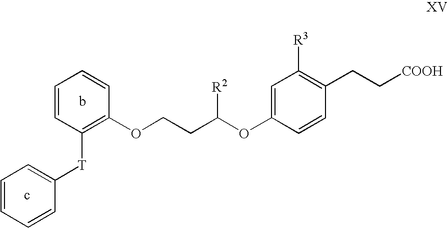
or a pharmaceutically acceptable salt, solvate, hydrate or stereoisomer thereof, wherein:
T is: a bond, O or C(O);
R is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, N(CH₃)₂, S(O)₂CH₃, methyl, ethyl, isopropyl, methoxy and cyclopropyl.

19. The compound of claim 2, wherein the compound is represented by structural formula XVI, 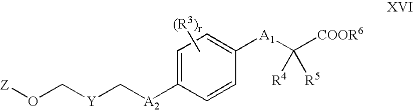
or a pharmaceutically acceptable salt, solvate, hydrate or stereoisomer thereof, wherein Y is a branched alkyl or C₃-C₆ cycloalkyl.

20. (canceled)

21. The compound of claim 19, wherein the compound structural formula XVIII, 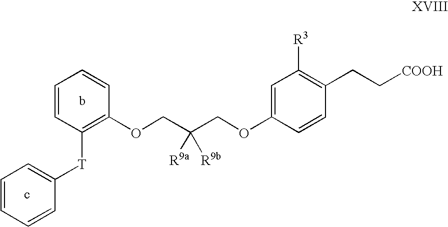
or a pharmaceutically acceptable salt, solvate, hydrate or stereoisomer thereof, wherein:
T is: a bond, O or C(O);
R³ is: methyl or ethyl;
R^{9a} and R^{9b} are each independently hydrogen, methyl or ethyl, wherein at least one of R^{9a} and R^{9b} being methyl or ethyl;
rings b and c are each optionally substituted with one or more substituent independently selected from the group consisting of: hydrogen, Cl, Br, CF₃, OCF₃, S(O)₂CH₃, N(CH₃)₂, methyl, ethyl, isopropyl, methoxy and cyclopropyl.

22. (canceled)

23. The compound of claim 1, wherein the compound is a compound of formula XX, 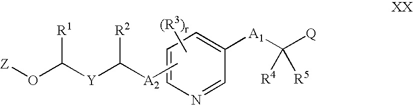
or a pharmaceutically acceptable salt, solvate, hydrate or stereoisomer thereof, wherein:
A₁ is: a bond, CH₂, O or S, and wherein A₁ and R⁴ or A₁ and R⁵ together being a 3- to 6-membered carbocyclyl when A₁ is a carbon;
A₂ is: O or S or CH₂;
Q is: —C(O)OR⁶, or R^{6A};
Y is: a bond, C₁-C₆ alkyl or C₃-C₆ cycloalkyl;
Z is: a) aryl;
b) a 5- to 10-membered heteroaryl wherein the heteroaryl containing at least one heteroatom selected from N, O or S,
c) bi-aryl, wherein biaryl being defined as aryl substituted with another aryl or aryl substituted with heteroaryl, or
d) bi-heteroaryl, wherein bi-heteroaryl being defined as heteroaryl substituted with another heteroaryl, or heteroaryl substituted with aryl, and
wherein aryl, heteroaryl, bi-aryl and bi-heteroaryl being optionally substituted with one or more groups independently selected from R⁷;
n is: 1,2,3,4, 5 or 6
p is: 1 or 2;
r is: 1,2,3, or 4;
R¹ and R² are each independently:
hydrogen,
haloalkyl,
C₁-C₆ alkyl,
(CH₂)ₙC₃-C₈ cycloalkyl, or
R¹ and R² form a 4- to 8-membered nonaromatic carbocyclic ring; and wherein at least one of R¹ and R² is alkyl or cycloalkyl, and;
R³ is: hydrogen,
nitro,
cyano,
hydroxyl,
halo,
haloalkyl,
haloalkyloxy,
aryloxy,
C₁-C₆ alkyl,
C₁-C₆ alkoxy, or
C₃-C₈ cycloalkyl;
R⁴ and R⁵ are each independently: hydrogen or C₁-C₆ alkyl;
R⁶ is: hydrogen, C₁-C₆ alkyl or aminoalkyl;
R^{6A} is: carboxamide, sulfonamide, acylsulfonamide, tetrazole, 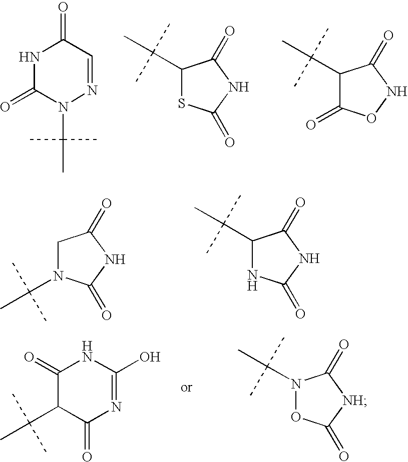
R⁷ is: hydrogen,
oxo,
nitro,
cyano,
hydroxyl,
halo,
haloalkyl,
haloalkyloxy,
aryloxy,
arylalkyl,
aminoalkyl,
C₁-C₆ alkyl
C₁-C₆ alkoxy,
(CH₂)ₙC₃-C₈ cycloalkyl,
C(O)R⁹,
C(O)OR⁹,
C(═NOR⁸)R⁹,
CR⁸(OH)R⁹,
C[═C(R⁸)₂]R⁹,
OR⁹,
SR⁹ or
S(O)ₚR⁹;
R⁸ is: hydrogen or C₁-C₆ alkyl; and
R⁹ is: hydrogen,
C₁-C₆ alkyl,
C₃-C₈ cycloalkyl,
aryl,
heteroaryl or
heterocyclyl,
wherein alkyl, cycloalkyl, aryl, heteroaryl or heterocyclyl being optionally substituted with one or more substituents selected from the group consisting of:
hydrogen, nitro, cyano, hydroxyl, halo, haloalkyl, haloalkyloxy, aryloxy, oxo, C₁-C₆ alkyl, C₁-C₆ alkoxy and C₃-C₈ cycloalkyl.

24. (canceled)

25. (canceled)

26. The compound of claim 23, wherein the compound is a compound of structural formula XXII, 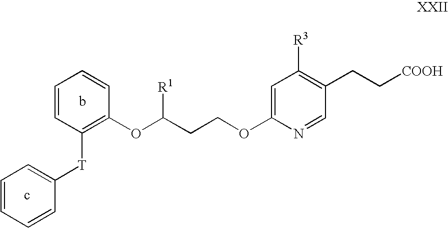
or a pharmaceutically acceptable salt, solvate, hydrate or stereoisomer thereof, wherein:
T is: a bond, —O— or —C(O)—;
R¹ is: methyl, ethyl or cyclopropyl;
R³ is: methyl or ethyl; and
rings b and c are each optionally substituted with one or more substituent independently selected from the group consisting of: hydrogen, Cl, Br, CF₃, OCF₃, S(O)₂CH₃, N(CH₃)₂, methyl, ethyl, isopropyl, methoxy and cyclopropyl.

27. The compound of claim 1, wherein the compound is a compound of structural formula XXIII, 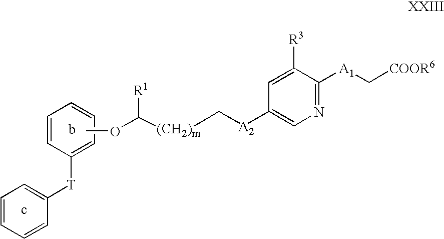
or a pharmaceutically acceptable salt, solvate, hydrate or stereoisomer thereof, wherein:
A₁ and A₂ are respectively:
O and O,
CH₂ and O,
CH₂ and S,
O and S or
S and O;
m is: 1,2, 3 or 4;
R¹ is: C₁-C₃ alkyl; and
R³ is: hydrogen, halo or C₁-C₆ alkyl;
R⁶ and R⁹ are each independently: hydrogen or C₁-C₆ alkyl;
T is: a bond, —O—, —C(O)—, —S(O)—S(O)₂—, —C(═CH₂)—, —C(═NOH)— or —CH(OH)—; and
rings b and c are each optionally substituted with one or more groups independently selected from:
hydrogen, oxo, nitro, cyano, hydroxyl, halo, haloalkyl, haloalkyloxy, aryloxy, arylalkyl, aminoalkyl, S(O)₂R⁹, C₁-C₆ alkyl, C₁-C₆ alkoxy and (CH₂)ₙC₃-C₈ cycloalkyl.

28. (canceled)

29. A compound of claim 1 selected from the group consisting of:

| No. | Structure | Name |
|---|---|---|
| 1 | 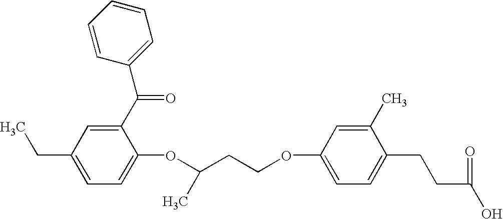 | 3-{4-[3-(2-Benzoyl-4- ethyl-phenoxy)-butoxy]- 2-methyl-phenyl}- propionic acid |
| 2 | 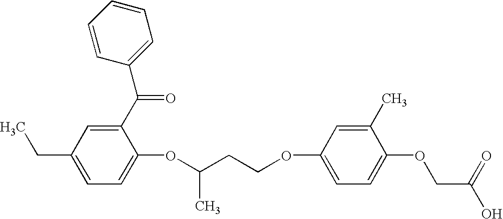 | {4-[3-(2-Benzoyl-4- ethyl-phenoxy)-butoxy]- 2-methyl-phenoxyl- acetic acid |
| 3 | 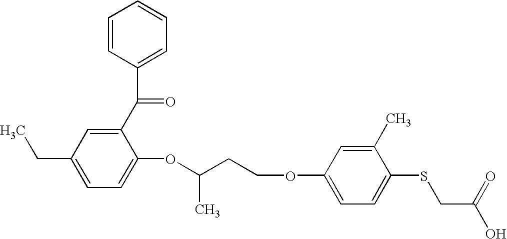 | {4-[3-(2-Benzoyl-4- ethyl-phenoxy)-butoxy]- 2-methyl- phenylsulfanyl}-acetic acid |
| 4 | 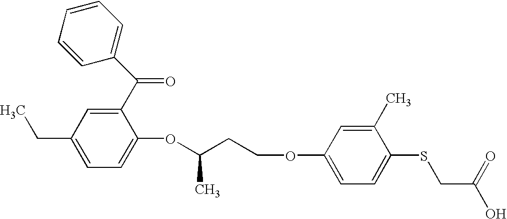 | {4-[3-(2-Benzoyl-4- ethyl-phenoxy)-butoxy]- 2-methyl- phenylsulfanyl}-acetic acid |
| 5 | 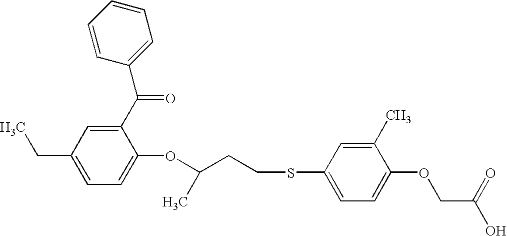 | {4-[3-(2-Benzoyl-4- ethyl-phenoxy)- butylsulfanyl]-2-methyl- phenoxy}-acetic acid |
| 6 | 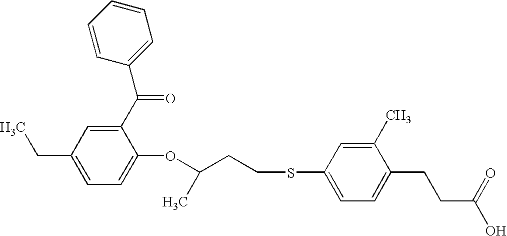 | 3-{4-[3-(2-Benzoyl-4- ethyl-phenoxy)- butylsulfanyl]-2-methyl- phenyl}-propionic acid |
| 7 | 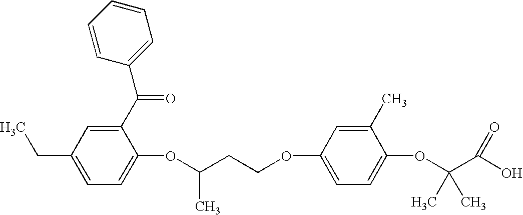 | 2-{4-[3-(2-Benzoyl-4- ethyl-phenoxy)-butoxy]- 2-methyl-phenoxy}-2- methyl-propionic acid |
| 8 | 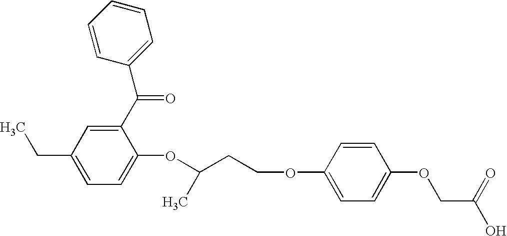 | {4-[3-(2-Benzoyl-4- ethyl-phenoxy)-butoxy]- phenoxy}-acetic acid |
| 9 | 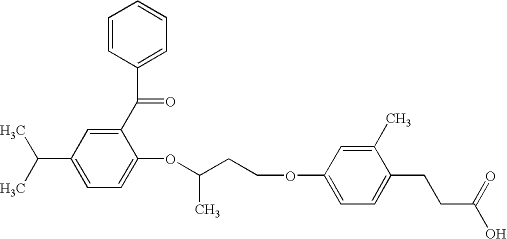 | 3-{4-[3-(2-Benzoyl-4- isopropyl-phenoxy)- butoxy]-2-methyl- phenyl}-propionic acid |
| 10 | 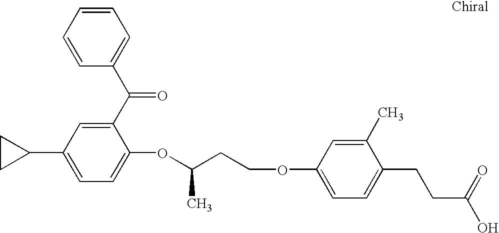 | 3-{4-[3-(2-Benzoyl-4- cyclopropyl-phenoxy)- butoxy]-2-methyl- phenyl}-propionic acid |
| 11 | 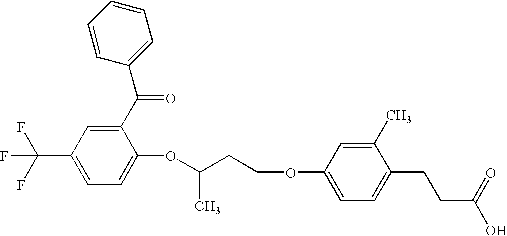 | 3-{4-[3-(2-Benzoyl-4- trifluoromethyl- phenoxy)-butoxy]-2- methyl-phenyl}- propionic acid |
| 12 | 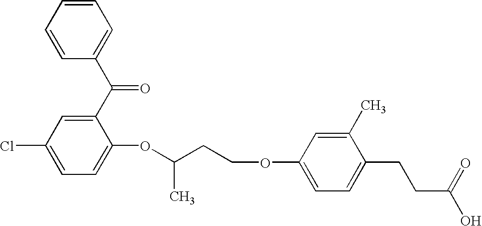 | 3-{4-[3-(2-Benzoyl-4- chloro-phenoxy)- butoxy]-2-methyl- phenyl}-propionic acid |
| 13 | 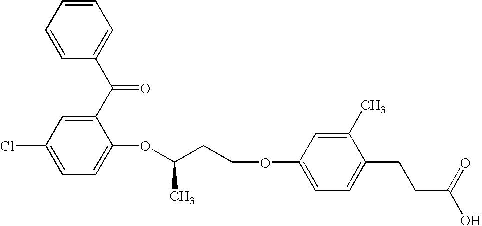 | 3-{4-[3-(2-Benzoyl-4- chloro-phenoxy)- butoxy]-2-methyl- phenyl}-propionic acid |
| 14 | 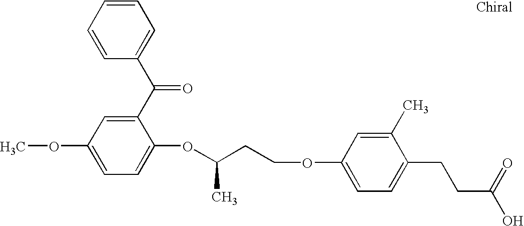 | 3-{4-[3-(2-Benzoyl-4- methoxy-phenoxy)- butoxy]-2-methyl- phenyl}-propionic acid |
| 15 | 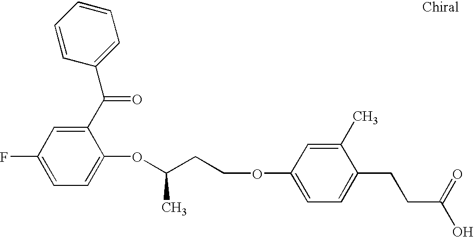 | 3-{4-[3-(2-Benzoyl-4- fluoro-phenoxy)- butoxy]-2-methyl- phenyl}-propionic acid |
| 16 | 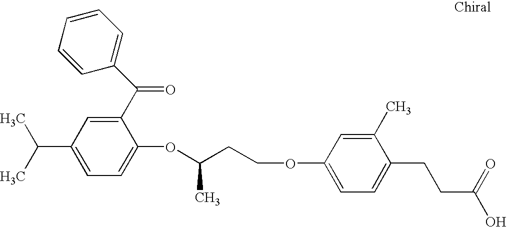 | 3-{4-[3-(2-Benzoyl-4- isopropyl-phenoxy)- butoxy]-2-methyl- phenyl}-propionic acid |
| 17 | 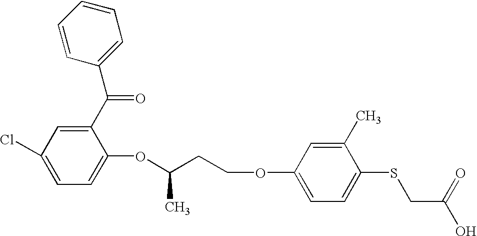 | {4-[3-(2-Benzoyl-4- chloro-phenoxy)- butoxy]-2-methyl- phenylsulfanyl}-acetic acid |
| 18 | 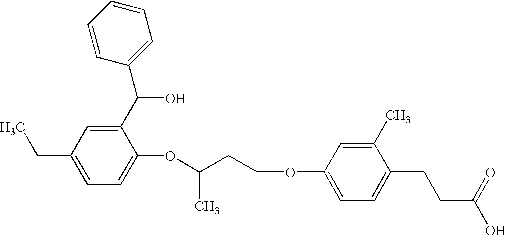 | 3-(4-{3-[4-Ethyl-2- (hydroxy-phenyl- methyl)-phenoxy]- butoxy}-2-methyl- phenyl)-propionic acid |
| 19 | 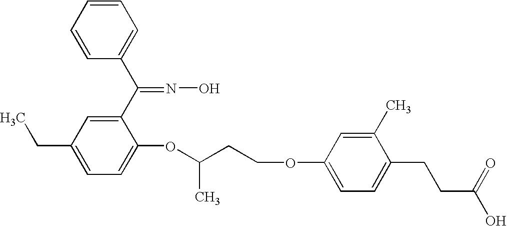 | 3-(4-{3-[4-Ethyl-2- (hydroxyimino-phenyl- methyl)-phenoxy]- butoxy}-2-methyl- phenyl)-propionic acid |
| 20 | 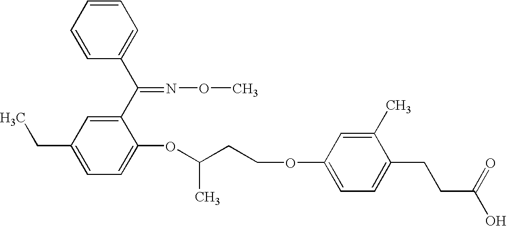 | 3-(4-{3-[4-Ethyl-2- (methoxyimino-phenyl- methyl)-phenoxy]- butoxy}-2-methyl- phenyl)-propionic acid |
| 21 | 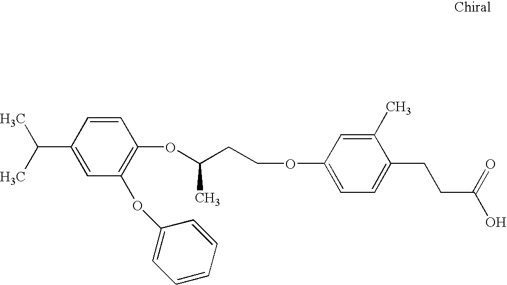 | 3-{4-[3-(4-Isopropyl-2- phenoxy-phenoxy)- butoxy]-2-methyl- phenyl}-propionic acid |
| 22 | 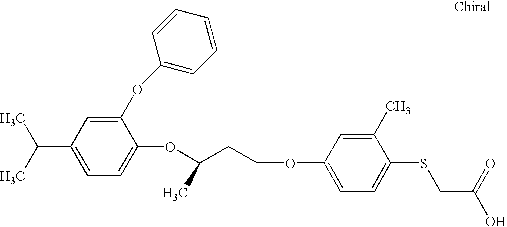 | {4-[3-(4-Isopropyl-2- phenoxy-phenoxy)- butoxy]-2-methyl- phenylsulfanyl}-acetic acid |
| 23 | 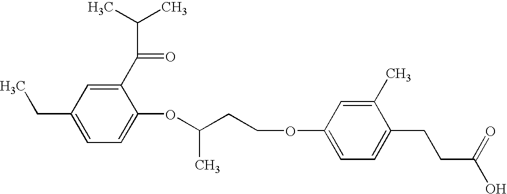 | 3-{4-[3-(4-Ethyl-2- isobutyryl-phenoxy)- butoxy]-2-methyl- phenyl}-propionic acid |
| 24 | 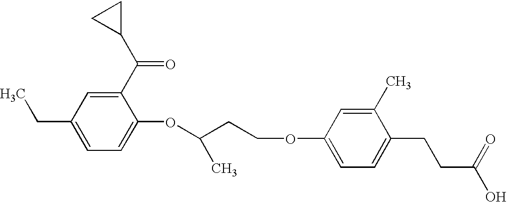 | 3-{4-[3-(2- Cyclopropanecarbonyl-4- ethyl-phenoxy)-butoxy]- 2-methyl-phenyl}- propionic acid |
| 25 | 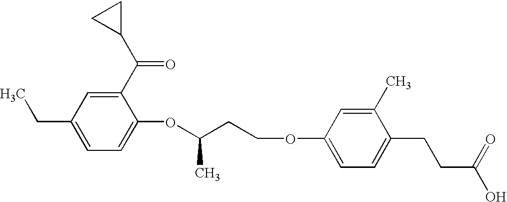 | 3-{4-[3-(2- Cyclopropanecarbonyl-4- ethyl-phenoxy)-butoxy]- 2-methyl-phenyl}- propionic acid |
| 26 | 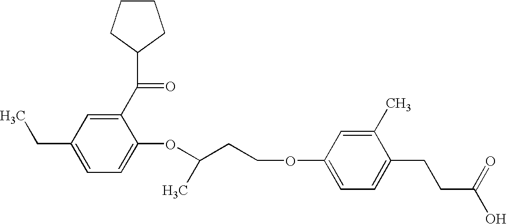 | 3-{4-[3-(2- Cyclopentanecarbonyl-4- ethyl-phenoxy)-butoxy]- 2-methyl-phenyl}- propionic acid |
| 27 | 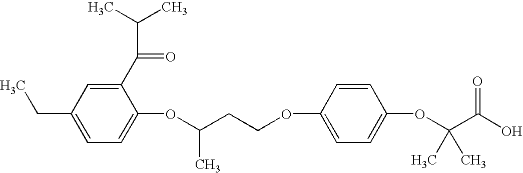 | 2-{4-[3-(4-Ethyl-2- isobutyryl-phenoxy)- butoxy]-phenoxy}-2- methyl-propionic acid |
| 28 | 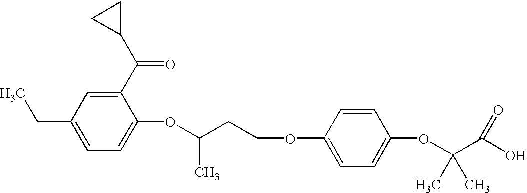 | 2-{4-[3-(2- Cyclopropanecarbonyl-4- ethyl-phenoxy)-butoxy]- phenoxy}-2-methyl- propionic acid |
| 29 | 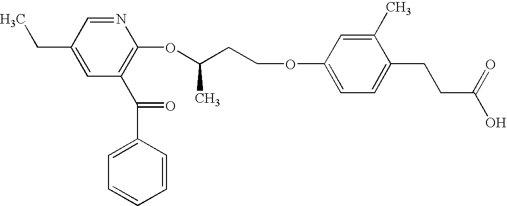 | 3-{4-[3-(3-Benzoyl-5- ethyl-pyridin-2-yloxy)- butoxyl-2-methyl- phenyl}-propionic acid |
| 30 | 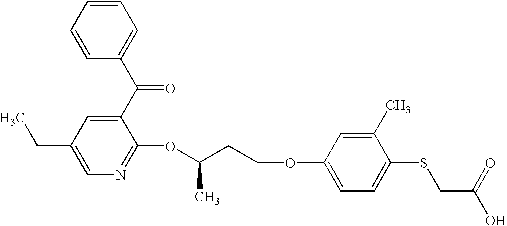 | {4-[3-(3-Benzoyl-5- ethyl-pyridin-2-yloxy)- butoxy]-2-methyl- phenylsulfanyl}-acetic acid |
| 31 | 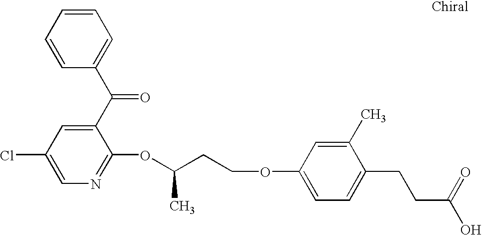 | 3-{4-[3-(3-Benzoyl-5- chloro-pyridin-2-yloxy)- butoxy]-2-methyl- phenyl}-propionic acid |
| 32 | 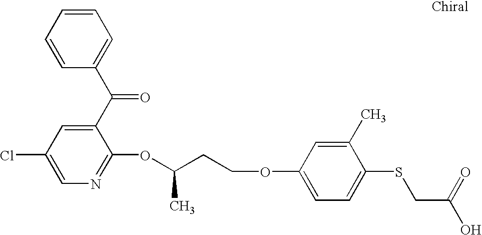 | {4-[3-(3-Benzoyl-5- chloro-pyridin-2-yloxy)- butoxy]-2-methyl- phenylsulfanyl}-acetic acid |
| 33 | 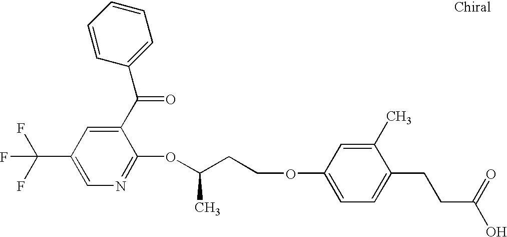 | 3-{4-[3-(3-Benzoyl-5- trifluoromethyl-pyridin- 2-yloxy)-butoxy]-2- methyl-phenyl}- propionic acid |
| 34 | 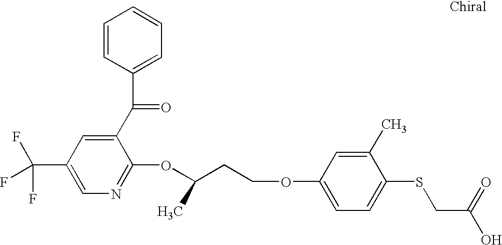 | {4-[3-(3-Benzoyl-5- trifluoromethyl-pyridin- 2-yloxy)-butoxy]-2- methyl-phenylsulfanyl}- acetic acid |
| 35 | 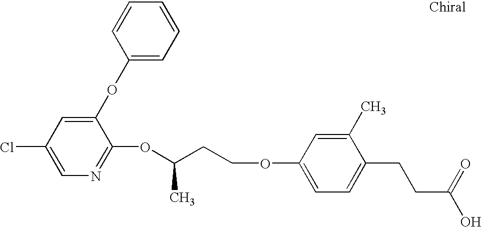 | 3-{4-[3-(5-Chloro-3- phenoxy-pyridin-2- yloxy)-butoxy]-2- methyl-phenyl}- propionic acid |
| 36 | 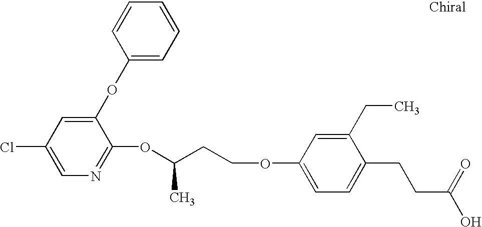 | 3-{4-[3-(5-Chloro-3- phenoxy-pyridin-2- yloxy)-butoxy]-2-ethyl- phenyl}-propionic acid |
| 37 | 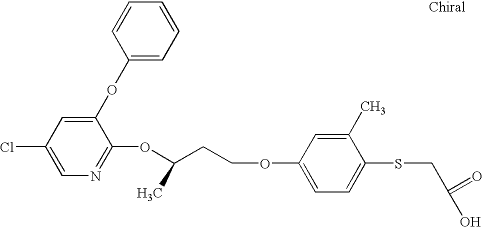 | {4-[3-(5-Chloro-3- phenoxy-pyridin-2- yloxy)-butoxy]-2- methyl-phenylsulfanyl}- acetic acid |
| 38 | 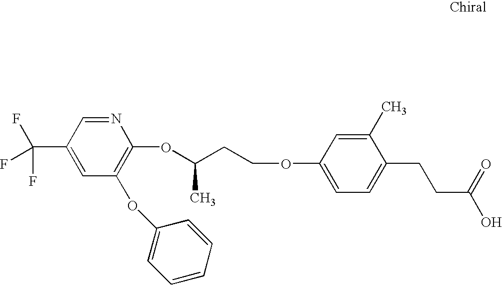 | 3-{2-Methyl-4-[3-(3- phenoxy-5- trifluoromethyl-pyridin- 2-yloxy)-butoxy]- phenyl}-propionic acid |
| 39 | 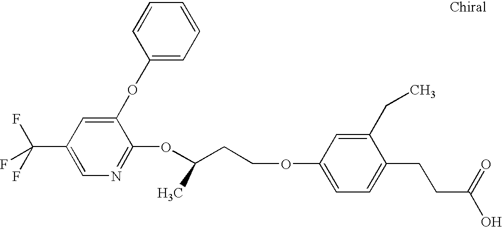 | 3-{2-Ethyl-4-[3-(3- phenoxy-5- trifluoromethyl-pyridin- 2-yloxy)-butoxyl- phenyl}-propionic acid |
| 40 | 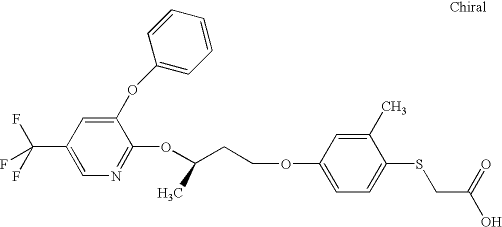 | 3-{2-Ethyl-4-[3-(3- phenoxy-5- trifluoromethyl-pyridin- 2-yloxy)-butoxy]- phenyl}-propionic acid |
| 41 | 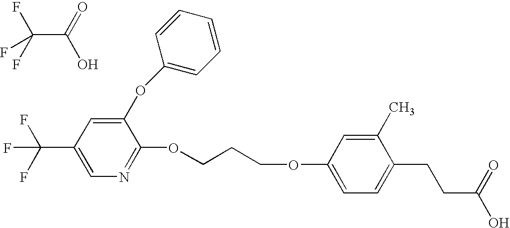 | 3-{2-Methyl-4-[3-(3- phenoxy-5- trifluoromethyl-pyridin- 2-yloxy)-propoxy]- phenyl}-propionic acid (trifluoroacetic acid salt) |
| 42 | 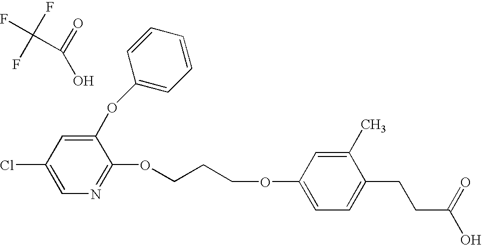 | 3-{4-[3-(5-Chloro-3- phenoxy-pyridin-2- yloxy)-propoxy]-2- methyl-phenyl}- propionic acid |
| 43 | 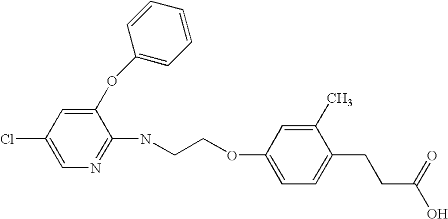 | 3-{4-[2-(5-Chloro-3- phenoxy-pyridin-2- ylamino)-ethoxyl-2- methyl-phenyl}- propionic acid |
| 44 | 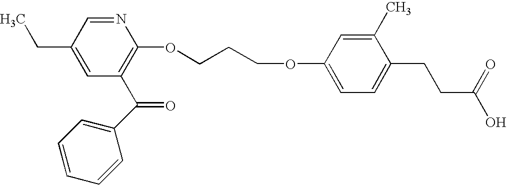 | 3-{4-[3-(3-Benzoyl-5- ethyl-pyridin-2-yloxy)- propoxy]-2-methyl- phenyl}-propionic acid |
| 45 | 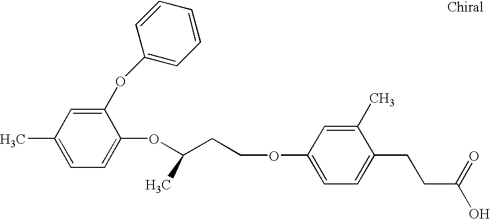 | 3-{2-Methyl-4-[3-(6- methyl-2-phenoxy- pyridin-3-yloxy)- butoxy]-phenyl}- propionic acid |
| 46 | 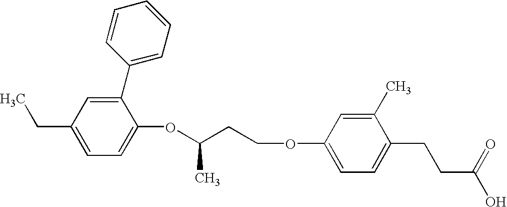 | 3-{4-[3-(5-Ethyl- biphenyl-2-yloxy)- butoxy]-2-methyl- phenyl}-propionic acid |
| 47 | 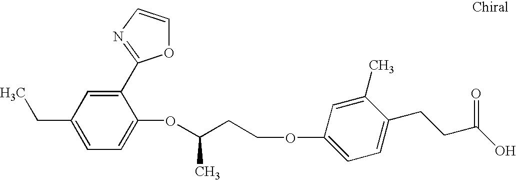 | 3-{4-[3-(4-Ethyl-2- oxazol-2-yl-phenoxy)- butoxyl-2-methyl- phenyl}-propionic acid |
| 48 | 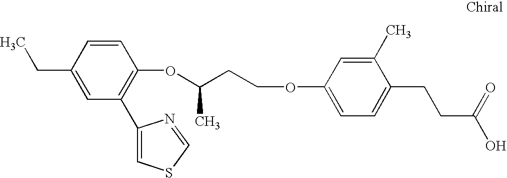 | 3-{4-[3-(4-Ethyl-2- thiazol-4-yl-phenoxy)- butoxy]-2-methyl- phenyl]-propionic acid |
| 49 | 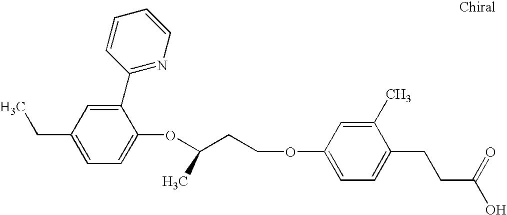 | 3-{4-[3-(4-Ethyl-2- pyridin-2-yl-phenoxy)- butoxy]-2-methyl- phenyl}-propionic acid |
| 50 | 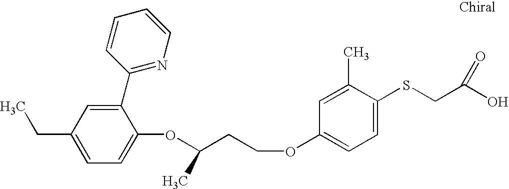 | {4-[3-(4-Ethyl-2-pyridin- 2-yl-phenoxy)-butoxy]- 2-methyl- phenylsulfanyl}-acetic acid |
| 51 | 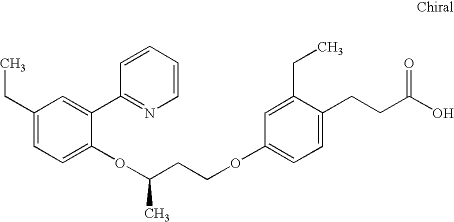 | 3-{2-Ethyl-4-[3-(4-ethyl- 2-pyridin-2-yl-phenoxy)- butoxy]-phenyl}- propionic acid |
| 52 | 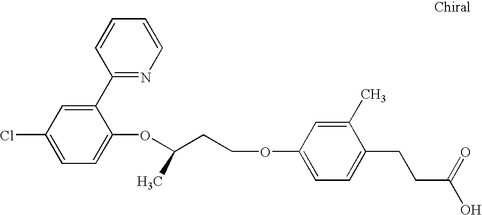 | 3-{4-[3-(4-Chloro-2- pyridin-2-yl-phenoxy)- butoxy]-2-methyl- phenyl}-propionic acid |
| 53 | 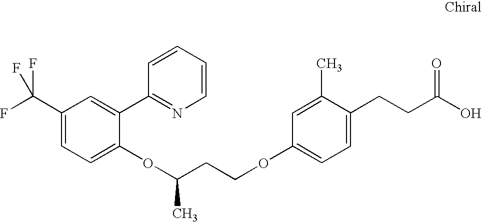 | 3-{2-Methyl-4-[3-(2- pyridin-2-yl-4- trifluoromethyl- phenoxy)-butoxy]- phenyl}-propionic acid |
| 54 | 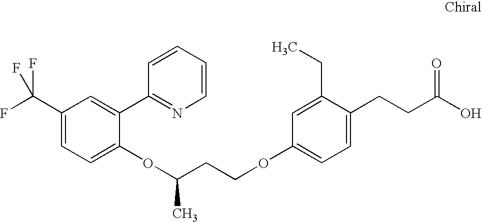 | 3-{2-Ethyl-4-[3-(2- pyridin-2-yl-4- trifluoromethyl- phenoxy)-butoxy]- phenyl}-propionic acid |
| 55 | 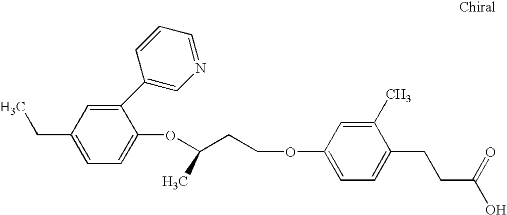 | 3-{4-[3-(4-Ethyl-2- pyridin-3-yl-phenoxy)- butoxy]-2-methyl- phenyl}-propionic acid |
| 56 | 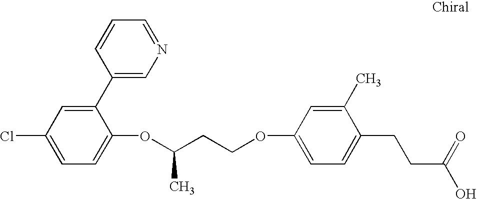 | 3-{4-[3-(4-Chloro-2- pyridin-3-yl-phenoxy)- butoxy]-2-methyl- phenyl}-propionic acid |
| 57 | 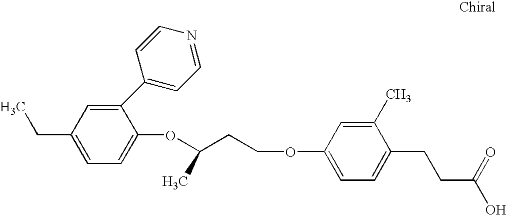 | 3-{4-[3-(4-Ethyl-2- pyridin-4-yl-phenoxy)- butoxy]-2-methyl- phenyl}-propionic acid |
| 58 | 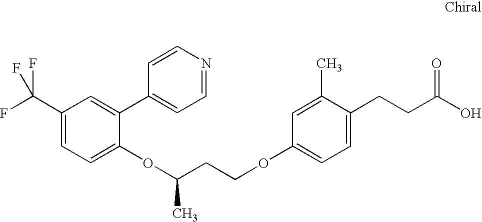 | 3-{2-Methyl-4-[3-(2- pyridin-4-yl-4- trifluoromethyl- phenoxy)-butoxy]- phenyl}-propionic acid |
| 59 | 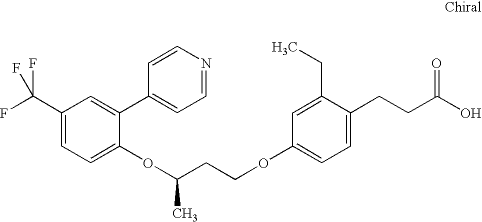 | 3-{2-Ethyl-4-[3-(2- pyridin-4-yl-4- trifluoromethyl- phenoxy)-butoxy]- phenyl}-propionic acid |
| 60 | 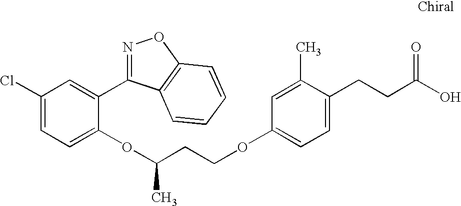 | 3-{4-[3-(2- Benzo[d]isoxazol-3-yl-4- chloro-phenoxy)- butoxy]-2-methyl- phenyl}-propionic acid |
| 61 | 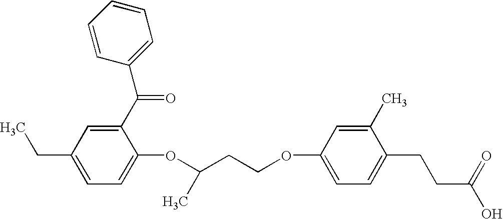 | 3-{4-[3-(2-Benzoyl-4- ethyl-phenoxy)-butoxy]- 2-methyl-phenyl}- propionic acid |
| 62 | 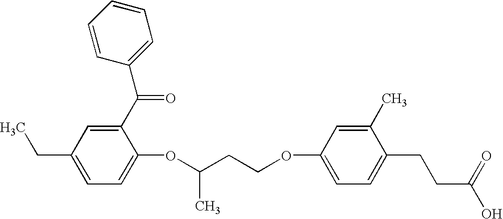 | {4-[3-(2-Benzoyl-4- ethyl-phenoxy)-butoxy]- 2-methyl-phenoxy}- acetic acid |
| 63 | 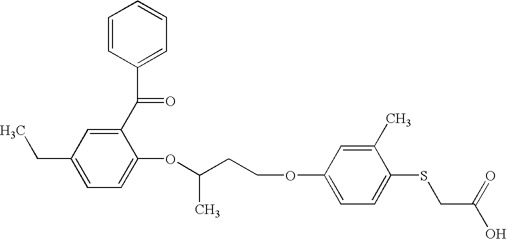 | {4-[3-(2-Benzoyl-4- ethyl-phenoxy)-butoxy]- 2-methyl- phenylsulfanyl}-acetic acid |
| 64 | 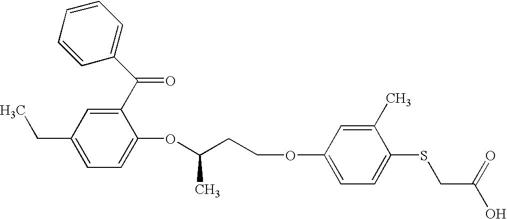 | {4-[3-(2-Benzoyl-4- ethyl-phenoxy)-butoxy]- 2-methyl- phenylsulfanyl}-acetic acid |
| 65 | 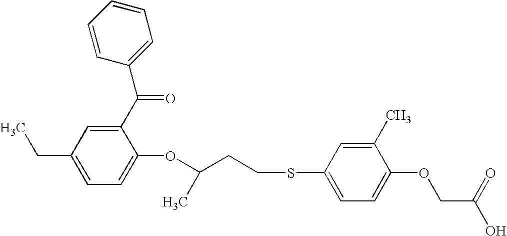 | {4-[3-(2-Benzoyl-4- ethyl-phenoxy)- butylsulfanyl]-2-methyl- phenoxy}-acetic acid |
| 66 | 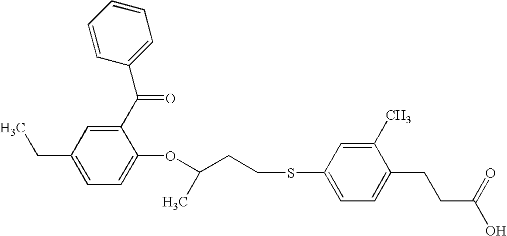 | 3-{4-[3-(2-Benzoyl-4- ethyl-phenoxy)- butylsulfanyl]-2-methyl- phenyl}-propionic acid |
| 67 | 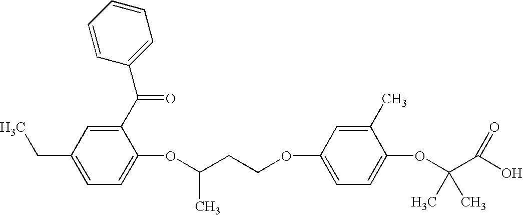 | 2-{4-[3-(2-Benzoyl-4- ethyl-phenoxy)-butoxy]- 2-methyl-phenoxy}-2- methyl-propionic acid |
| 68 | 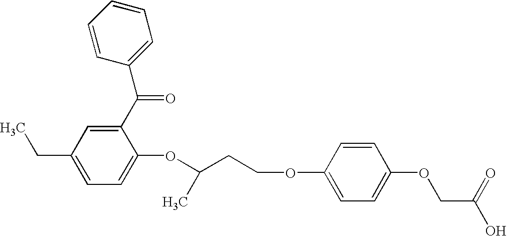 | {4-[3-(2-Benzoyl-4- ethyl-phenoxy)-butoxy]- phenoxy}-acetic acid |
| 69 | 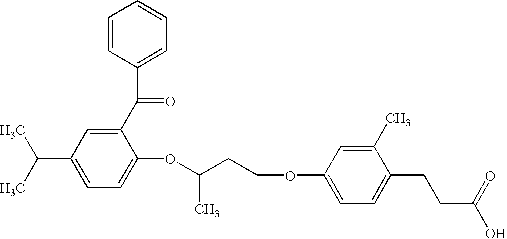 | 3-{4-[3-(2-Benzoyl-4- isopropyl-phenoxy)- butoxy]-2-methyl- phenyl}-propionic acid |
| 70 | 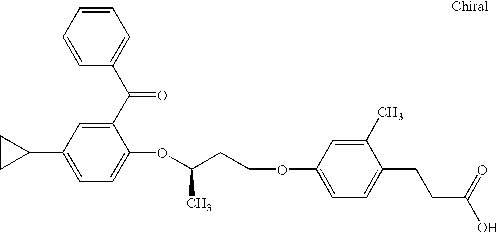 | 3-{4-[3-(2-Benzoyl-4- cyclopropyl-phenoxy)- butoxy]-2-methyl- phenyl}-propionic acid |
| 71 | 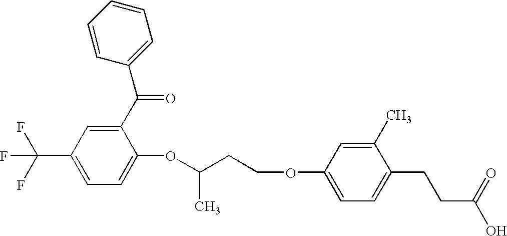 | 3-{4-[3-(2-Benzoyl-4- trifluoromethyl- phenoxy)-butoxy]-2- methyl-phenyl}- propionic acid |
| 72 | 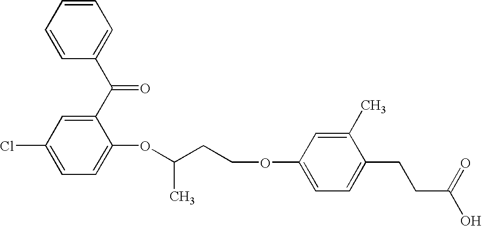 | 3- {4-[3-(2-Benzoyl-4- chloro-phenoxy)- butoxy]-2-methyl- phenyl}-propionic acid |
| 73 | 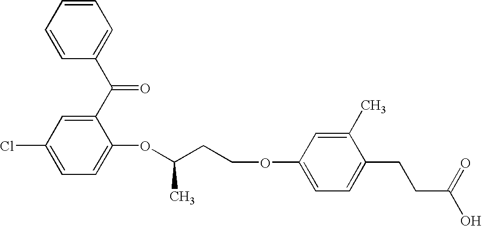 | 3-{4-[3-(2-Benzoyl-4- chloro-phenoxy)- butoxy]-2-methyl- phenyl}-propionic acid |
| 74 | 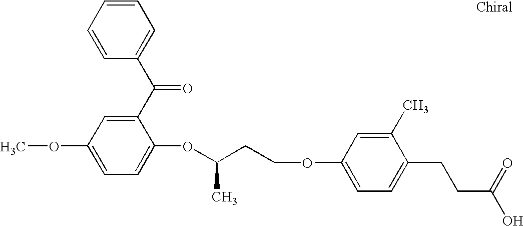 | 3-{4-[3-(2-Benzoyl-4- methoxy-phenoxy)- butoxy]-2-methyl- phenyl}-propionic acid |
| 75 | 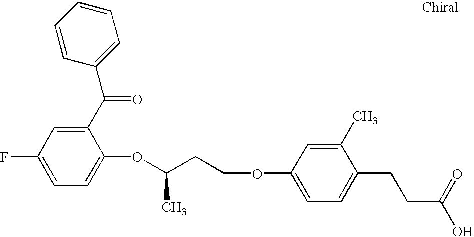 | 3-{4-[3-(2-Benzoyl-4- fluoro-phenoxy)- butoxy]-2-methyl- phenyl}-propionic acid |
| 76 | 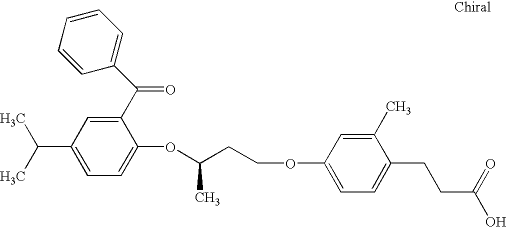 | 3-{4-[3-(2-Benzoyl-4- isopropyl-phenoxy)- butoxy]-2-methyl- phenyl}-propionic acid |
| 77 | 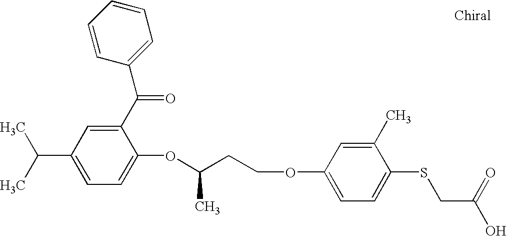 | {4-[3-(2-Benzoyl-4- isopropyl-phenoxy)- butoxy]-2-methyl- phenylsulfanyl}-acetic acid |
| 78 | 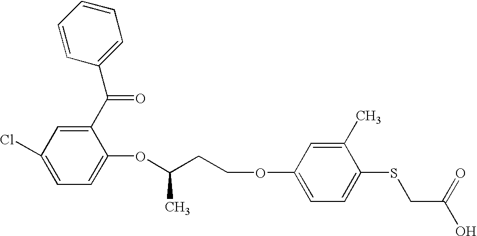 | {4-[3-(2-Benzoyl-4- chloro-phenoxy)- butoxy]-2-methyl- phenylsulfanyl}-acetic acid |
| 79 | 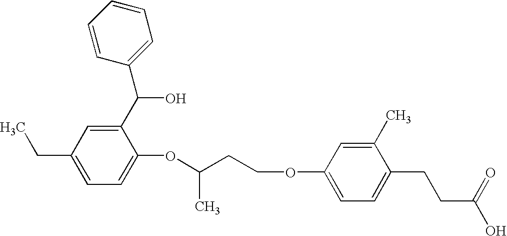 | 3-(4-{3-[4-Ethyl-2- (hydroxy-phenyl- methyl)-phenoxy]- butoxy}-2-methyl- phenyl)-propionic acid |
| 80 | 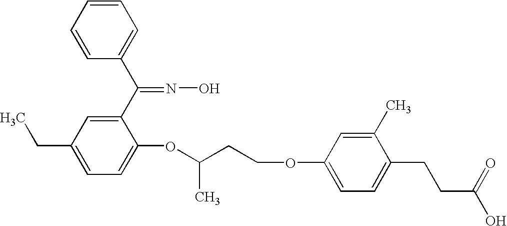 | 3-(4-{3-[4-Ethyl-2- (hydroxyimino-phenyl- methyl)-phenoxy]- butoxy}-2-methyl- phenyl)-propionic acid |
| 81 | 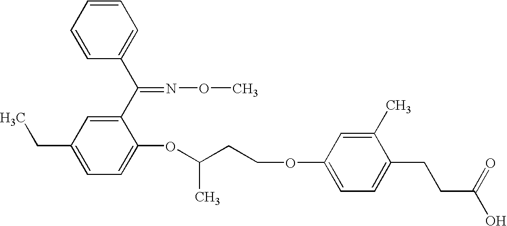 | 3-(4-{3-[4-Ethyl-2- (methoxyimino-phenyl- methyl)-phenoxy]- butoxy}-2-methyl- phenyl)-propionic acid |
| 82 | 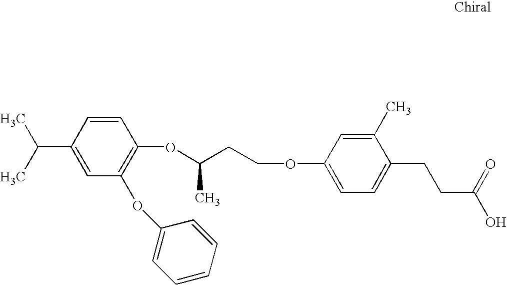 | 3-{4-[3-(4-Isopropyl-2- phenoxy-phenoxy)- butoxy]-2-methyl- phenyl}-propionic acid |
| 83 | 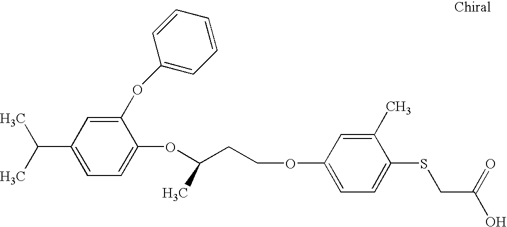 | {4-[3-(4-Isopropyl-2- phenoxy-phenoxy)- butoxy]-2-methyl- phenylsulfanyl}-acetic acid |
| 84 | 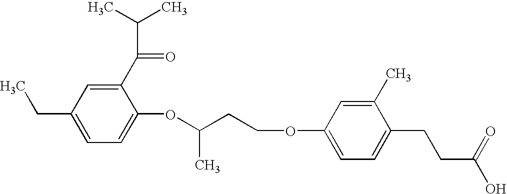 | 3-{4-[3-(4-Ethyl-2- isobutyryl-phenoxy)- butoxy]-2-methyl- phenyl}-propionic acid |
| 85 | 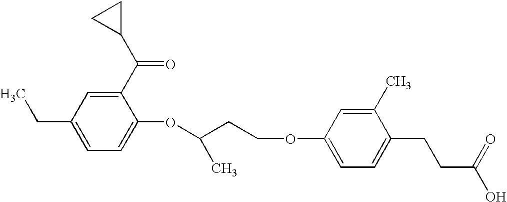 | 3-{4-[3-(2- Cyclopropanecarbonyl-4- ethyl-phenoxy)-butoxy]- 2-methyl-phenyl}- propionic acid |
| 86 | 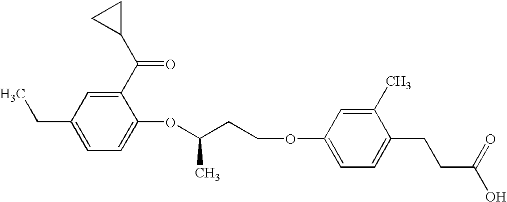 | 3-{4-[3-(2- Cyclopropanecarbonyl-4- ethyl-phenoxy)-butoxy]- 2-methyl-phenyl}- propionic acid |
| 87 | 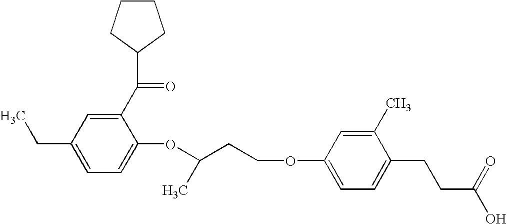 | 3-{4-[3-(2- Cyclopentanecarbonyl-4- ethyl-phenoxy)-butoxy]- 2-methyl-phenyl}- propionic acid |
| 88 | 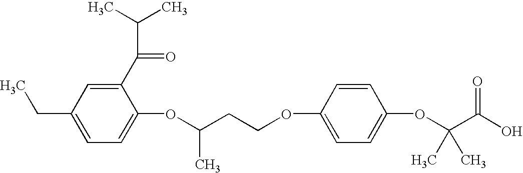 | 2-{4-[3-(4-Ethyl-2- isobutyryl-phenoxy)- butoxy]-phenoxy}-2- methyl-propionic acid |
| 89 | 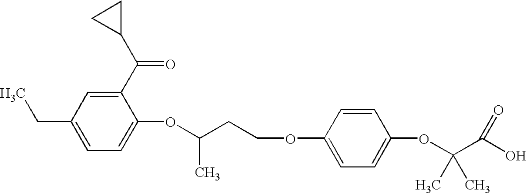 | 2-{4-[3-(2- Cyclopropanecarbonyl-4- ethyl-phenoxy)-butoxy]- phenoxy}-2-methyl- propionic acid |
| 90 | 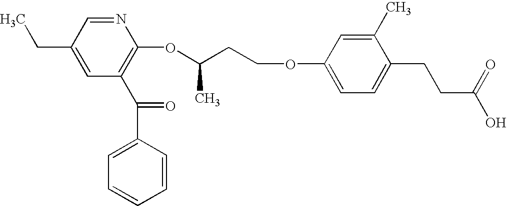 | 3-{4-[3-(3-Benzoyl-5- ethyl-pyridin-2-yloxy)- butoxy]-2-methyl- phenyl}-propionic acid |
| 91 | 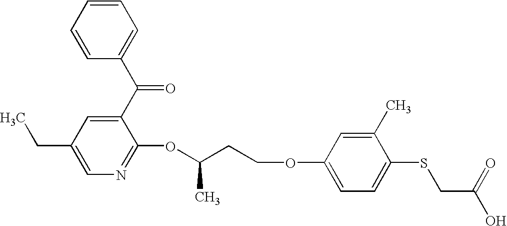 | {4-[3-(3-Benzoyl-5- ethyl-pyridin-2-yloxy)- butoxy]-2-methyl- phenylsulfanyl}-acetic acid |
| 92 | 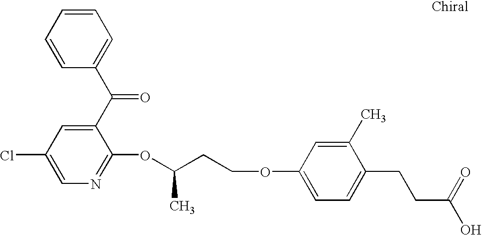 | 3-{4-[3-(3-Benzoyl-5- chloro-pyridin-2-yloxy)- butoxy]-2-methyl- phenyl}-propionic acid |
| 93 | 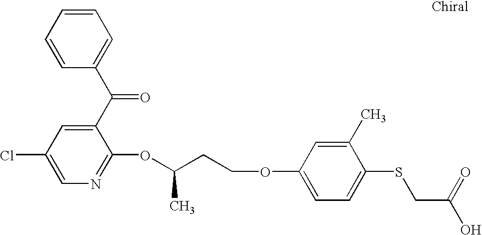 | {4-[3-(3-Benzoyl-5- chloro-pyridin-2-yloxy)- butoxy]-2-methyl- phenylsulfanyl}-acetic acid |
| 94 | 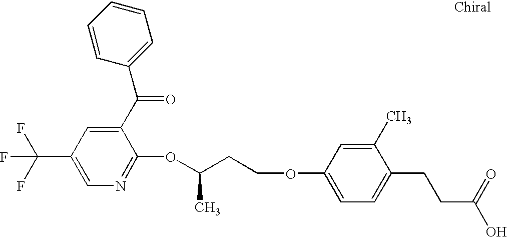 | 3-{4-[3-(3-Benzoyl-5- trifluoromethyl-pyridin- 2-yloxy)-butoxy]-2- methyl-phenyl}- propionic acid |
| 95 | 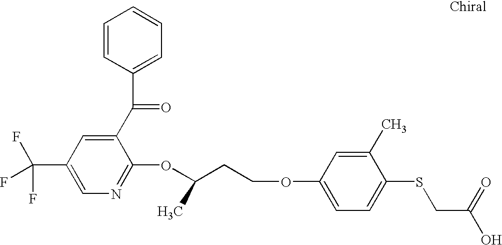 | {4-[3-(3-Benzoyl-5- trifluoromethyl-pyridin- 2-yloxy)-butoxy]-2- methyl-phenylsulfanyl}- acetic acid |
| 96 | 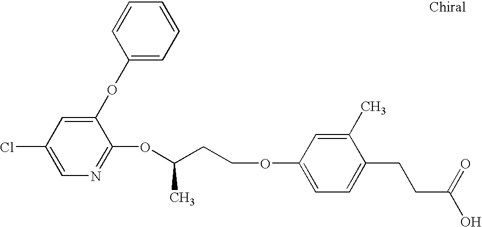 | 3-{4-[3-(5-Chloro-3- phenoxy-pyridin-2- yloxy)-butoxy]-2- methyl-phenyl}- propionic acid |
| 97 | 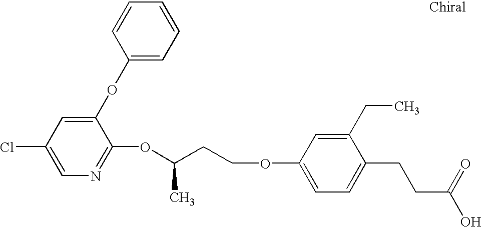 | 3-{4-[3-(5-Chloro-3- phenoxy-pyridin-2- yloxy)-butoxy]-2-ethyl- phenyl}-propionic acid |
| 98 | 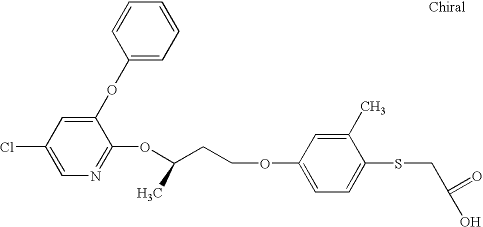 | {4-[3-(5-Chloro-3- phenoxy-pyridin-2- yloxy)-butoxy]-2- methyl -phenylsulfanyl}- acetic acid |
| 99 | 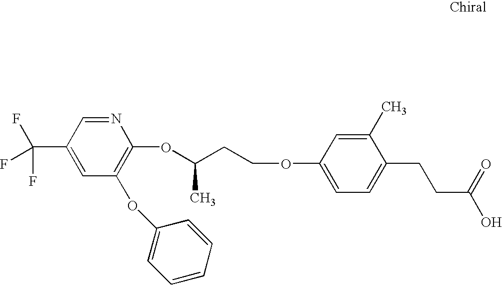 | 3-{2-Methyl-4-[3-(3- phenoxy-5- trifluoromethyl-pyridin- 2-yloxy)-butoxy]- phenyl}-propionic acid |
| 100 | 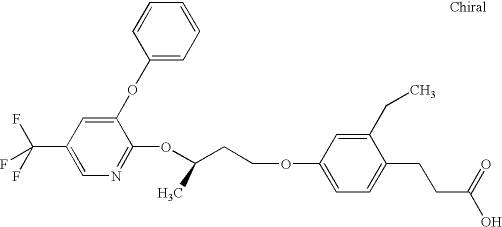 | 3-{2-Ethyl-4-[3-(3- phenoxy-5- trifluoromethyl-pyridin- 2-yloxy)-butoxy]- phenyl}-propionic acid |
| 101 | 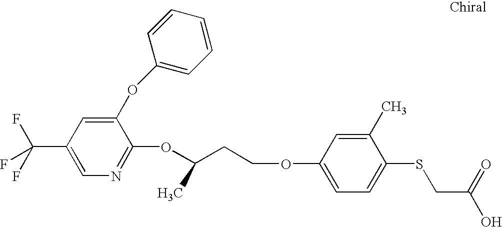 | 3-{2-Ethyl-4-[3-(3- phenoxy-5- trifluoromethyl-pyridin- 2-yloxy)-butoxy]- phenyl}-propionic acid |
| 102 | 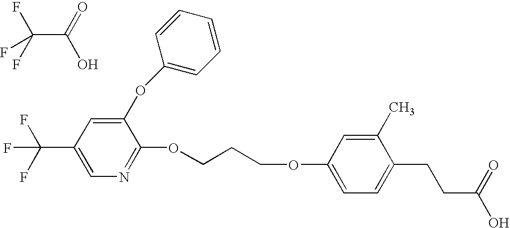 | 3-{2-Methyl-4-[3-(3- phenoxy-5- trifluoromethyl-pyridin- 2-yloxy)-propoxy]- phenyl}-propionic acid (trifluoroacetic acid salt) |
| 103 | 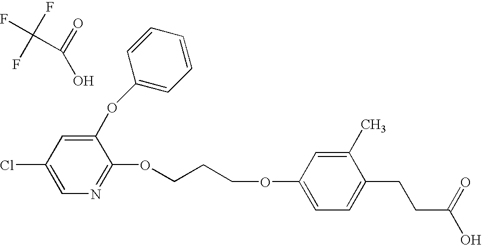 | 3-{4-[3-(5-Chloro-3- phenoxy-pyridin-2- yloxy)-propoxy]-2- methyl-phenyl}- propionic acid |
| 104 | 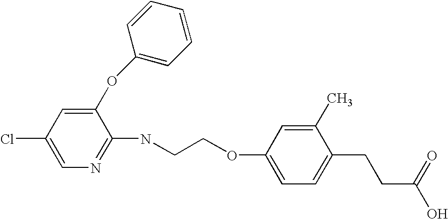 | 3-{4-[2-(5-Chloro-3- phenoxy-pyridin-2- ylamino)-ethoxy]-2- methyl-phenyl}- propionic acid |
| 105 | 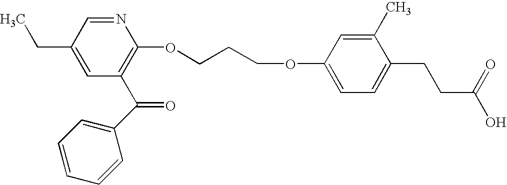 | 3-{4-[3-(3-Benzoyl-5- ethyl-pyridin-2-yloxy)- propoxyl-2-methyl- phenyl}-propionic acid |
| 106 | 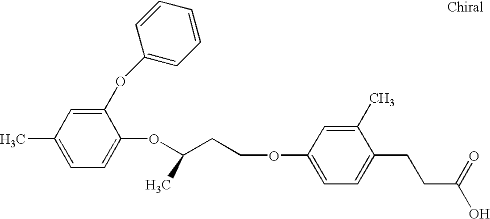 | 3-{2-Methyl-4-[3-(6- methyl-2-phenoxy- pyridin-3-yloxy)- butoxy]-phenyl}- propionic acid |
| 107 | 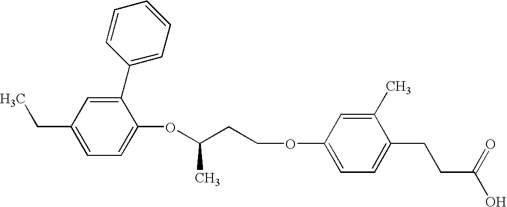 | 3-{4-[3-(5-Ethyl- biphenyl-2-yloxy)- butoxy]-2-methyl- phenyl}-propionic acid |
| 108 | 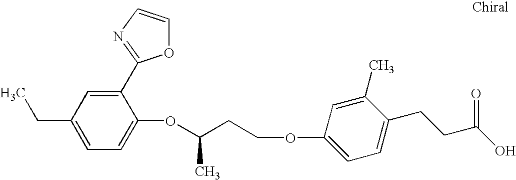 | 3-{4-[3-(4-Ethyl-2- oxazol-2-yl-phenoxy)- butoxy]-2-methyl- phenyl}-propionic acid |
| 109 | 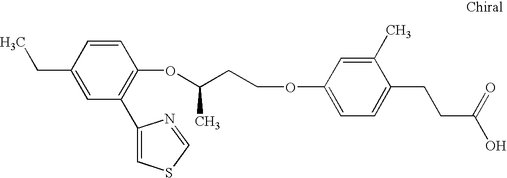 | 3-{4-[3-(4-Ethyl-2- thiazol-4-yl-phenoxy)- butoxy]-2-methyl- phenyl}-propionic acid |
| 110 | 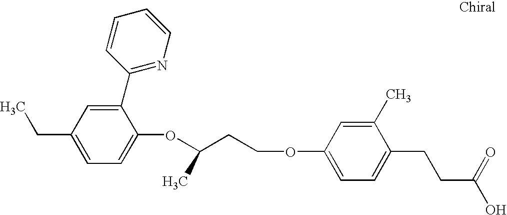 | 3-{4-[3-(4-Ethyl-2- pyridin-2-yl-phenoxy)- butoxy]-2-methyl- phenyl}-propionic acid |
| 111 | 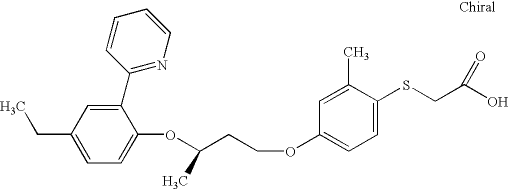 | {4-[3-(4-Ethyl-2-pyridin- 2-yl-phenoxy)-butoxy]- 2-methyl- phenylsulfanyl}-acetic acid |
| 112 | 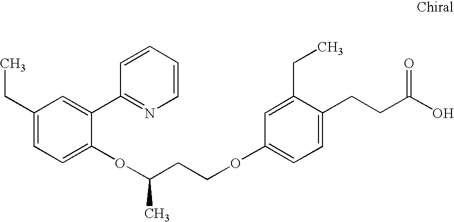 | 3-{2-Ethyl-4-[3-(4-ethyl- 2-pyridin-2-yl-phenoxy)- butoxy]-phenyl}- propionic acid |
| 113 | 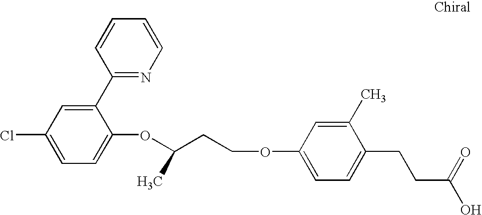 | 3-{4-[3-(4-Chloro-2- pyridin-2-yl-phenoxy)- butoxy]-2-methyl- phenyl}-propionic acid |
| 114 | 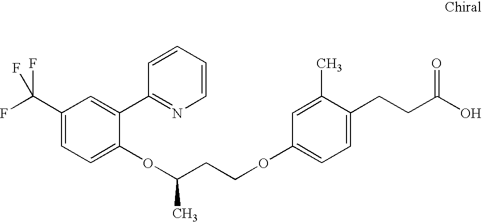 | 3-{2-Methyl-4-[3-(2- pyridin-2-yl-4- trifluoromethyl- phenoxy)-butoxy]- phenyl}-propionic acid |
| 115 | 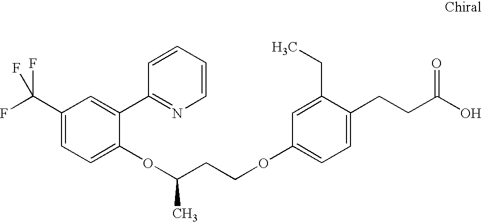 | 3-{2-Ethyl-4-[3-(2- pyridin-2-yl-4- trifluoromethyl- phenoxy)-butoxy]- phenyl}-propionic acid |
| 116 | 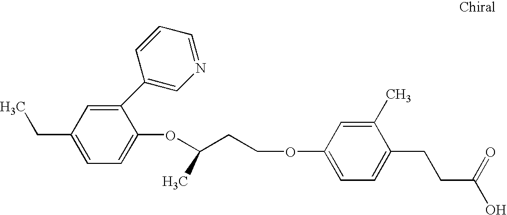 | 3-{4-[3-(4-Ethyl-2- pyridin-3-yl-phenoxy)- butoxy]-2-methyl- phenyl}-propionic acid |
| 117 | 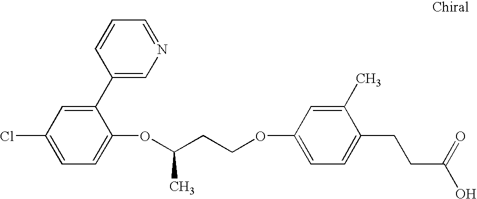 | 3-{4-[3-(4-Chloro-2- pyridin-3-yl-phenoxy)- butoxy]-2-methyl- phenyl}-propionic acid |
| 118 | 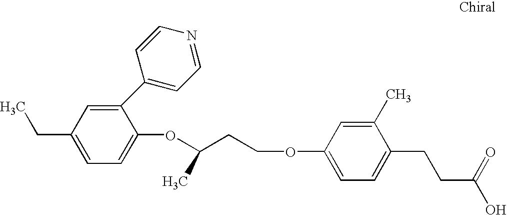 | 3-{4-[3-(4-Ethyl-2- pyridin-4-yl-phenoxy)- butoxy]-2-methyl- phenyl}-propionic acid |
| 119 | 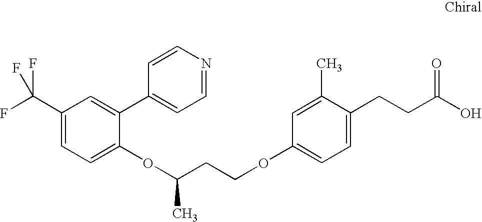 | 3-{2-Methyl-4-[3-(2- pyridin-4-yl-4- trifluoromethyl- phenoxy)-butoxy]- phenyl}-propionic acid |
| 120 | 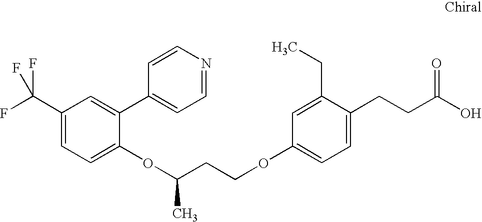 | 3-{2-Ethyl-4-[3-(2- pyridin-4-yl-4- trifluoromethyl- phenoxy)-butoxy]- phenyl}-propionic acid |
| 121 | 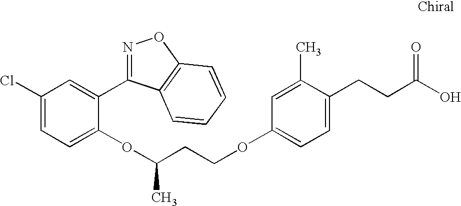 | 3-{4-[3-(2- Benzo[d]isoxazol-3-yl-4- chloro-phenoxy)- butoxy]-2-methyl- phenyl}-propionic acid |
| 122 | 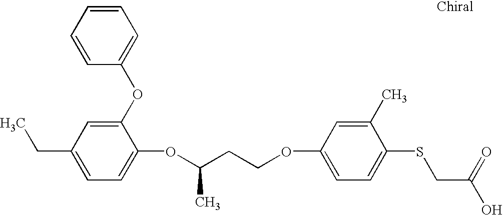 | (R)-{4-[3-(4-ethyl-2- phenoxy-phenoxy)- butoxy]-2-methyl- phenylsulfanyl}-acetic acid |
| 123 | 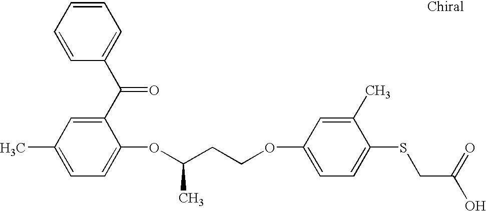 | (R)-{4-[3-(2-benzoyl-4- methyl-phenoxy)- butoxy]-2-methyl- phenylsulfanyl}-acetic acid |
| 124 | 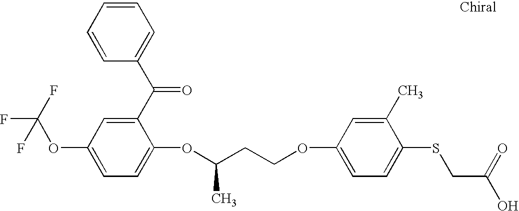 | (R)-{4-[3-(2-benzoyl-4- trifluoromethoxy- phenoxy)-butoxy]-2- methyl-phenylsulfanyl}- acetic acid |
| 125 | 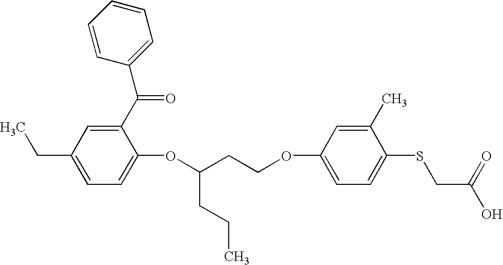 | {4-[3-(2-benzoyl-4-ethyl- phenoxy)-hexyloxy]-2- methyl-phenylsulfanyl}- acetic acid |
| 126 | 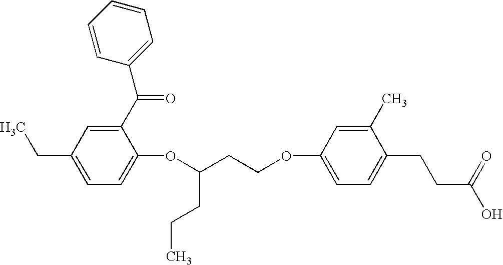 | 3-{4-[3-(2-benzoyl-4- ethyl-phenoxy)- hexyloxy]-2-methyl- phenyl}-propionic acid |
| 127 | 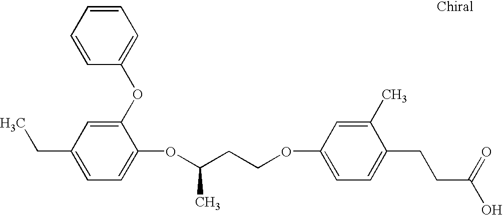 | (R)-3-{4-[3-(4-ethyl-2- phenoxy-phenoxy)- butoxy]-2-methyl- phenyl}-propionic acid |
| 128 | 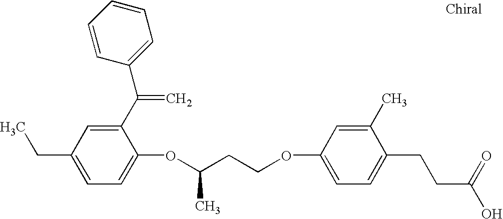 | (R)-3-(4-{3-[4-ethyl-2- (1-phenyl-vinyl)- phenoxy]-butoxy}-2- methyl-phenyl)- propionic acid |
| 129 | 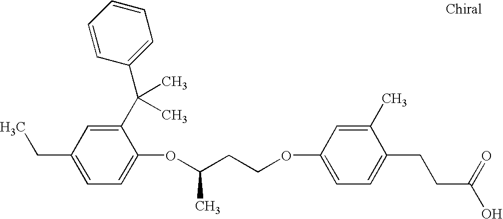 | (R)-3-(4-{3-[4-ethyl-2- (1-methyl-1-phenyl- ethyl)-phenoxy]- butoxy}-2-methyl- phenyl)-propionic acid |
| 130 | 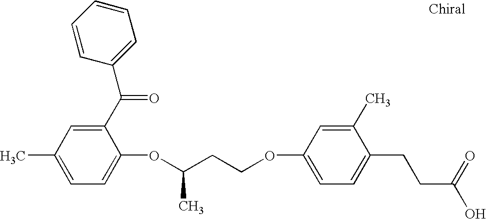 | (R)-3-{4-[3-(2-benzoyl- 4-methyl-phenoxy)- butoxy]-2-methyl- phenyl}-propionic acid |
| 131 | 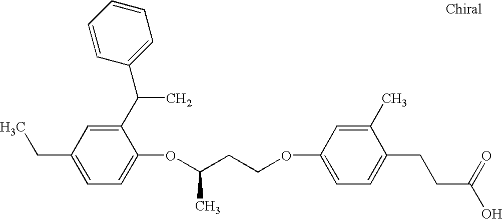 | (R)-3-(4-{3-[4-ethyl-2- (1-phenyl-ethyl)- phenoxy]-butoxy}-2- methyl-phenyl)- propionic acid |
| 132 | 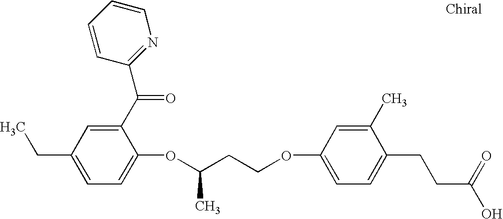 | (R)-3-(4-{3-[4-ethyl-2- (pyridine-2-carbonyl)- phenoxy]-butoxy}-2- methyl-phenyl)- propionic acid |
| 133 | 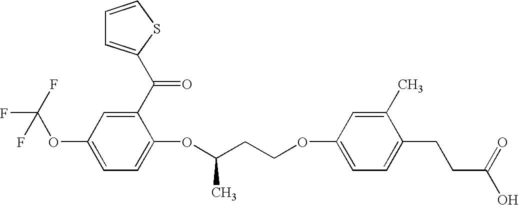 | 3-(2-methyl-4-{3-[2- (thiophene-2-carbonyl)- 4-trifluoromethoxy- phenoxy]-butoxy}- phenyl)-propionic acid |
| 134 | 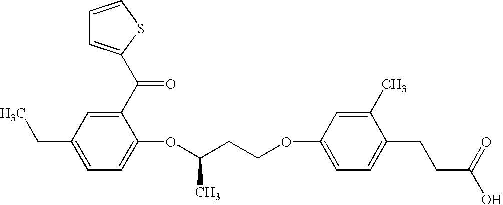 | 3-(4-{3-[4-ethyl-2- (thiophene-2-carbonyl)- phenoxyl-butoxy}-2- methyl-phenyl)- propionic acid |
| 135 | 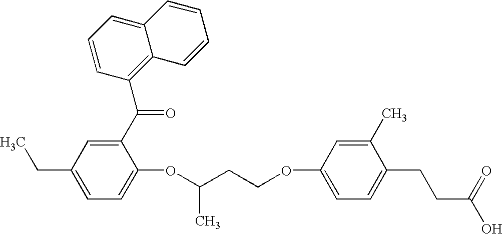 | 3-(4-{3-[4-ethyl-2- (naphthalene-1- carbonyl)-phenoxy]- butoxy}-2-methyl- phenyl)-propionic acid |
| 136 | 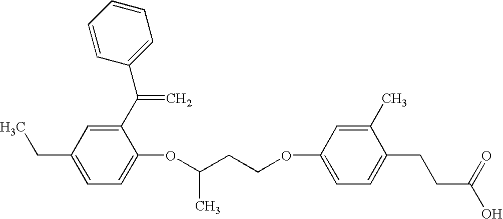 | 3-(4-{3-[4-ethyl-2-(1- phenyl-vinyl)-phenoxy]- butoxy}-2-methyl- phenyl)-propionic acid |
| 137 | 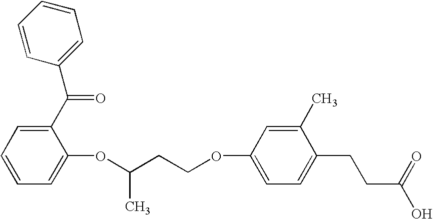 | 3-{4-[3-(2-benzoyl- phenoxy)-butoxy]-2- methyl-phenyl}- propionic acid |
| 138 | 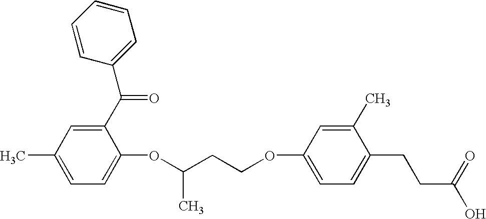 | 3-{4-[3-(2-benzoyl-4- methyl-phenoxy)- butoxy]-2-methyl- phenyl}-propionic acid |
| 139 | 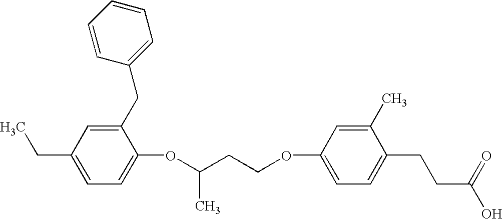 | 3-{4-[3-(2-benzyl-4- ethyl-phenoxy)-butoxy]- 2-methyl-phenyl}- propionic acid |
| 140 | 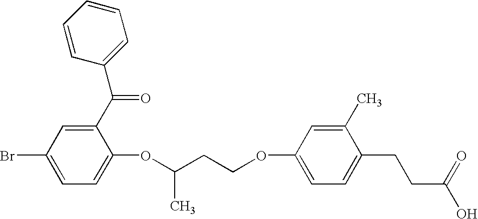 | 3-{4-[3-(2-benzoyl-4- bromo-phenoxy)- butoxy]-2-methyl- phenyl}-propionic acid |
| 141 | 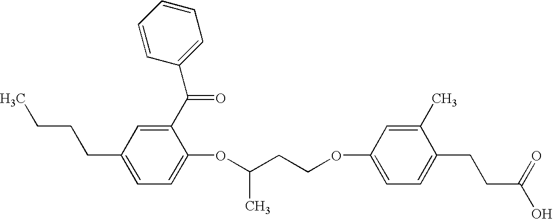 | 3-{4-[3-(2-benzoyl-4- butyl-phenoxy)-butoxy]- 2-methyl-phenyl}- propionic acid |
| 142 | 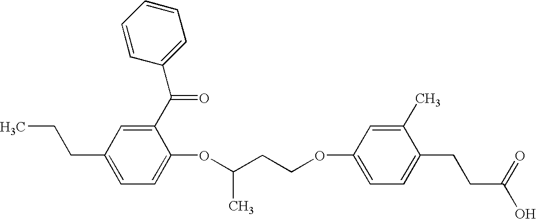 | 3-{4-[3-(2-benzoyl-4- propyl-phenoxy)- butoxy]-2-methyl- phenyl}-propionic acid |
| 143 | 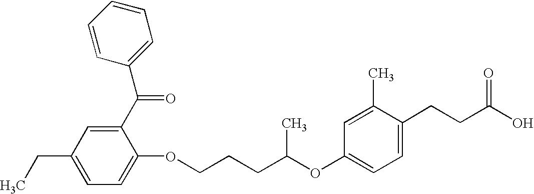 | 3-{4-[4-(2-benzoyl-4- ethyl-phenoxy)-1- methyl-butoxy]-2- methyl-phenyl}- propionic acid |
| 144 | 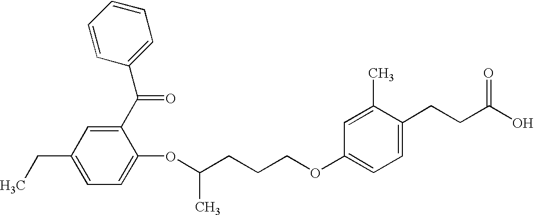 | 3-{4-[4-(2-benzoyl-4- ethyl-phenoxy)- pentyloxy]-2-methyl- phenyl}-propionic acid |
| 145 | 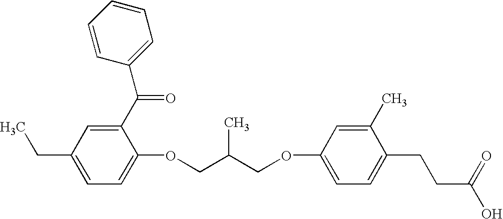 | 3-{4-[3-(2-benzoyl-4- ethyl-phenoxy)-2- methyl-propoxy]-2- methyl-phenyl}- propionic acid |
| 146 | 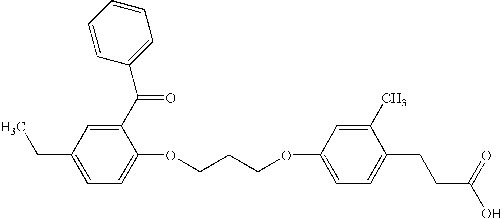 | 3-{4-[3-(2-benzoyl-4- ethyl-phenoxy)- propoxy]-2-methyl- phenyl}-propionic acid |
| 147 | 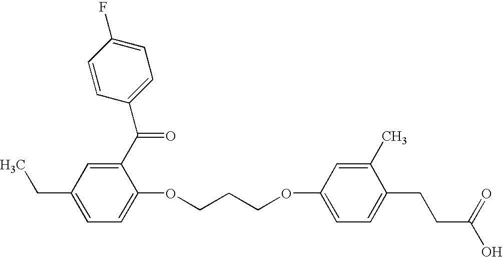 | 3-(4-{3-[4-ethyl-2-(4- fluoro-benzoyl)- phenoxy]-propoxy}-2- methyl-phenyl)- propionic acid |
| 148 | 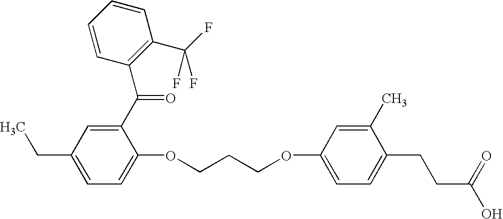 | 3-(4-{3-[4-ethyl-2-(2- trifluoromethyl-benzoyl)- phenoxy]-propoxy}-2- methyl-phenyl)- propionic acid |
| 149 | 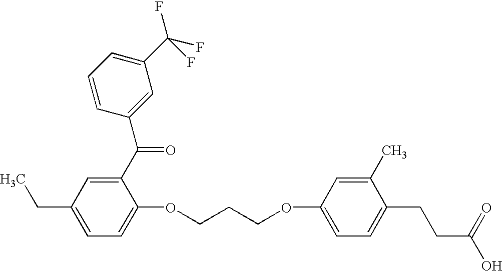 | 3-(4-{3-[4-ethyl-2-(3- trifluoromethyl-benzoyl)- phenoxy]-propoxy}-2- methyl-phenyl)- propionic acid |
| 150 | 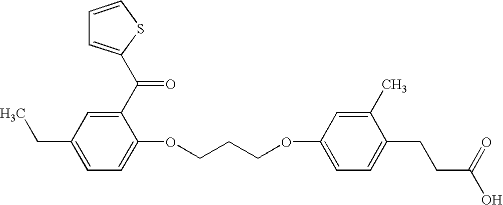 | 3-(4-{3-[4-ethyl-2- (thiophene-2-carbonyl)- phenoxy]-propoxy}-2- methyl-phenyl)- propionic acid |
| 151 | 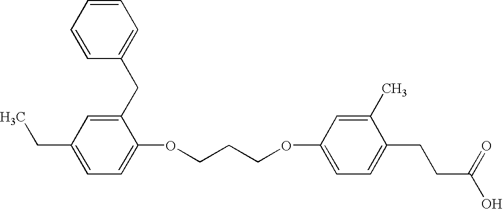 | 3-{4-[3-(2-benzyl-4- ethyl-phenoxy)- propoxy]-2-methyl- phenyl}-propionic acid |
| 152 | 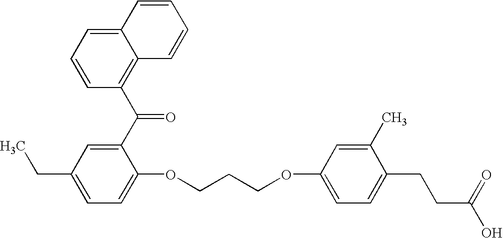 | 3-(4-{3-[4-ethyl-2- (naphthalene-1- carbonyl)-phenoxy]- propoxy}-2-methyl- phenyl)-propionic acid |
| 153 | 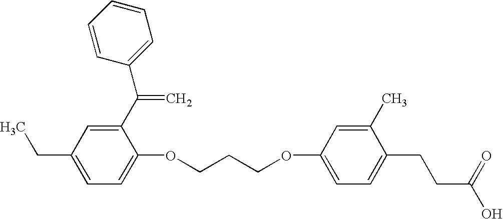 | 3-(4-{3-[4-ethyl-2-(1- phenyl-vinyl)-phenoxy]- propoxy}-2-methyl- phenyl)-propionic acid |
| 154 | 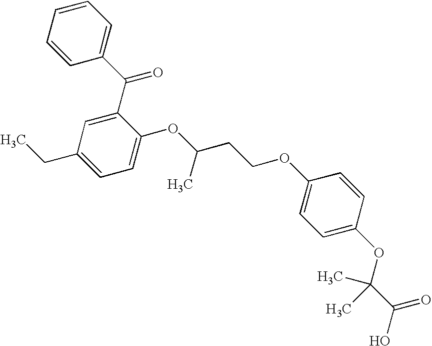 | 2-{4-[3-(2-benzoyl-4- ethyl-phenoxy)-butoxy]- phenoxy}-2-methyl- propionic acid |
| 155 | 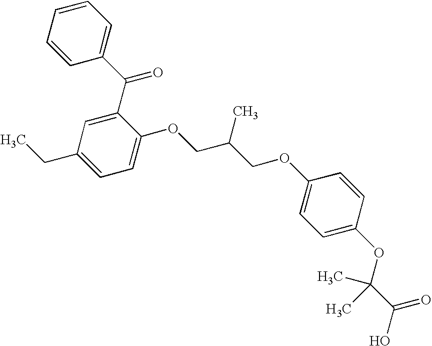 | 2-{4-[3-(2-benzoyl-4- ethyl-phenoxy)-2- methyl-propoxy]- phenoxy}-2-methyl- propionic acid |
| 156 | 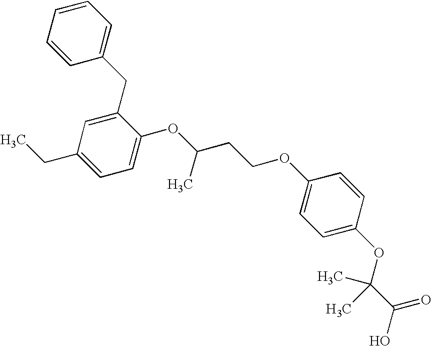 | 2-{4-[3-(2-benzyl-4- ethyl-phenoxy)-butoxy]- phenoxy}-2-methyl- propionic acid |
| 157 | 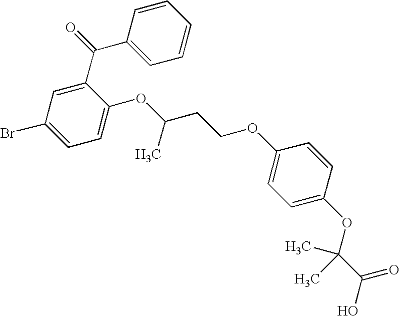 | 2-{4-[3-(2-benzoyl-4- bromo-phenoxy)- butoxy]-phenoxy}-2- methyl-propionic acid |
| 158 | 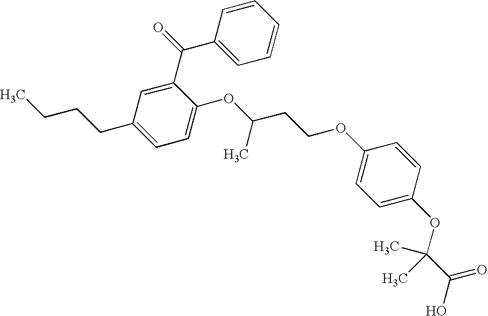 | 2-{4-[3-(2-benzoyl-4- butyl-phenoxy)-butoxy]- phenoxy}-2-methyl- propionic acid |
| 159 | 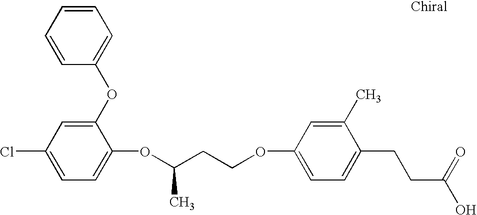 | (R)-3-{4-[3-(4-chloro-2- phenoxy-phenoxy)- butoxy]-2-methyl- phenyl}-propionic acid |
| 160 | 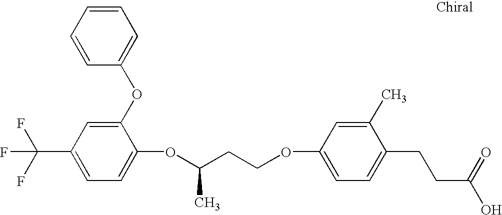 | (R)-3-{2-methyl-4-[3-(2- phenoxy-4- trifluoromethyl- phenoxy)-butoxy]- phenyl}-propionic acid |
| 161 | 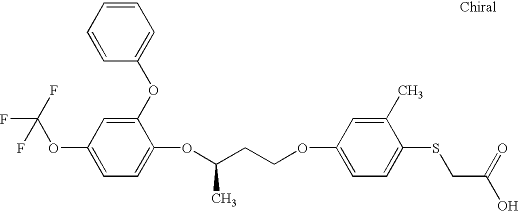 | (R)-3-{2-methyl-4-[3-(2- phenoxy-4- trifluoromethoxy- phenoxy)-butoxy]- phenyl}-propionic acid |
| 162 | 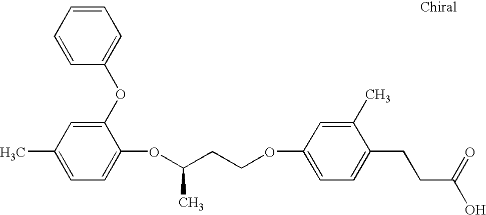 | (R)-3-{2-methyl-4-[3-(4- methyl-2-phenoxy- phenoxy)-butoxy]- phenyl}-propionic acid |
| 163 | 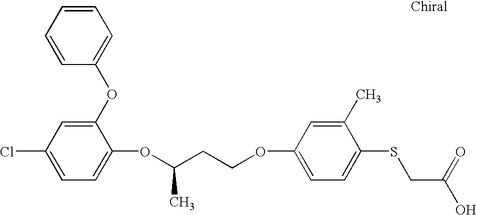 | (R)-{4-[3-(4-chloro-2- phenoxy-phenoxy)- butoxy]-2-methyl- phenylsulfanyl}-acetic acid |
| 164 | 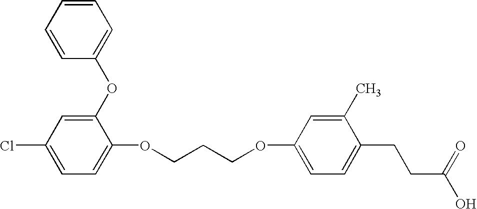 | 3-{4-[3-(4-chloro-2- phenoxy-phenoxy)- propoxyl-2-methyl- phenyl}-propionic acid |
| 165 | 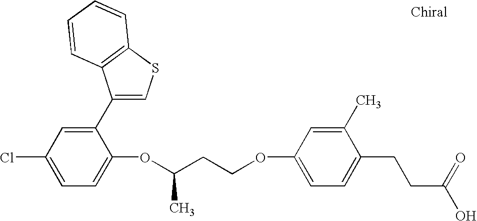 | (R)-3-{4-[3-(2- benzo[b]thiophen-3-yl-4- chloro-phenoxy)- butoxy]-2-methyl- phenyl}-propionic acid |
| 166 | 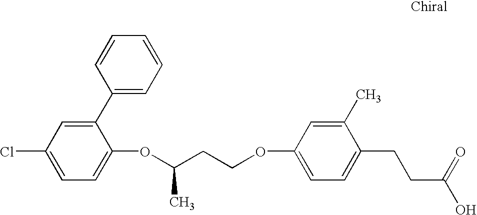 | (R)-3-{4-[3-(4-chloro-2- pyridin-3-yl-phenoxy)- butoxy]-2-methyl- phenyl}-propionic acid |
| 167 | 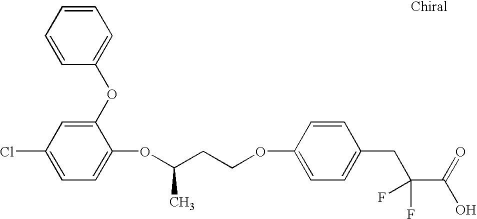 | (R)-3-{4-[3-(4-chloro-2- phenoxy-phenoxy)- butoxy]-phenyl}-2,2- difluoro-propionic acid |
| 168 | 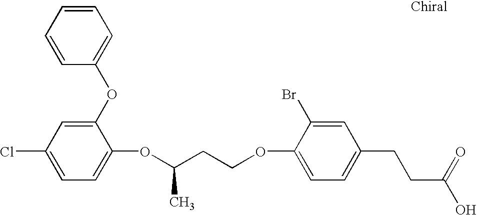 | (R)-3-{3-bromo-4-[3- (4-chloro-2-phenoxy- phenoxy)-butoxy]- phenyl}-propionic acid |
| 169 | 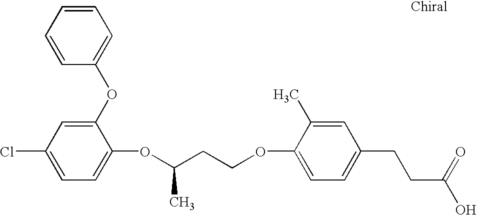 | (R)-3-{4-[3-(4-chloro-2- phenoxy-phenoxy)- butoxy]-3-methyl- phenyl}-propionic acid |
| 170 | 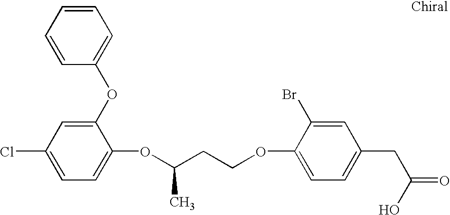 | (R)-{3-bromo-4-[3-(4- chloro-2-phenoxy- phenoxy)-butoxy]- phenyl}-acetic acid |
| 171 | 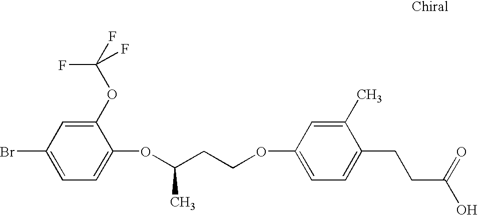 | (R)-3-{4-[3-(4-bromo-2- trifluoromethoxy- phenoxy)-butoxy]-2- methyl-phenyl}- propionic acid |
| 172 | 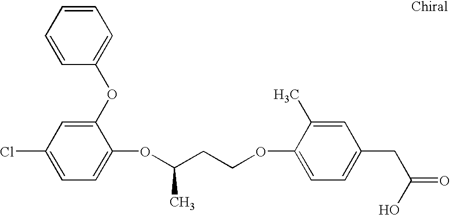 | (R)-{4-[3-(4-chloro-2- phenoxy-phenoxy)- butoxy]-3-methyl- phenyl}-acetic acid |
| 173 | 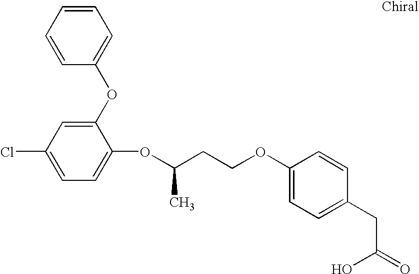 | (R)-{4-[3-(4-chloro-2- phenoxy-phenoxy)- butoxy]-phenyl}-acetic acid |
| 174 | 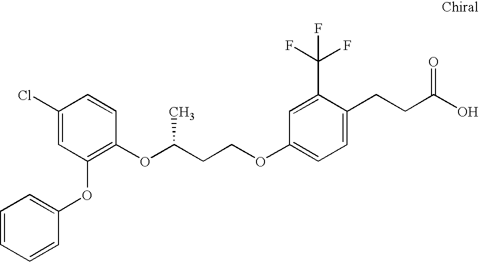 | (R)-3-{4-[3-(4-chloro-2- phenoxy-phenoxy)- butoxy]-2- trifluoromethyl-phenyl}- propionic acid |
| 175 | 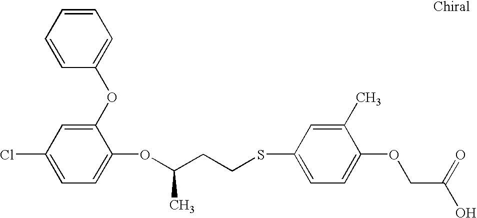 | (R)-{4-[3-(4-chloro-2- phenoxy-phenoxy)- butylsulfanyl]-2-methyl- phenoxy}-acetic acid |
| 176 | 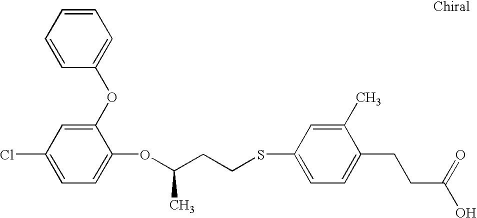 | (R)-3-{4-[3-(4-chloro-2- phenoxy-phenoxy)- butylsulfanyl]-2-methyl- phenyl}-propionic acid |
| 177 | 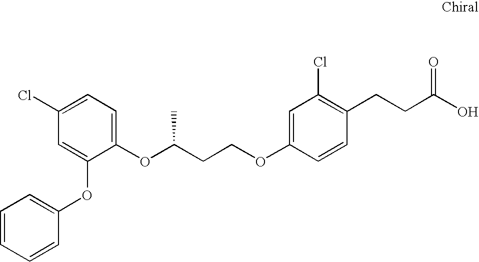 | (R)-3-{2-Chloro-4-[3-(4- chloro-2-phenoxy- phenoxy)-butoxy]- phenyl}-propionic acid |
| 178 | 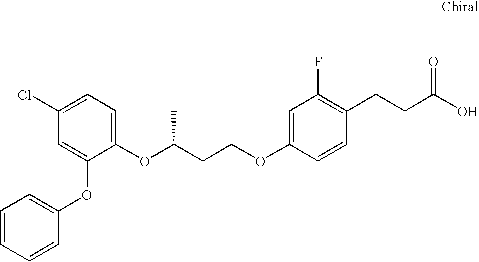 | (R)-3-{4-[3-(4-Chloro-2- phenoxy-phenoxy)- butoxy]-2-fluoro- phenyl}-propionic acid |
| 179 | 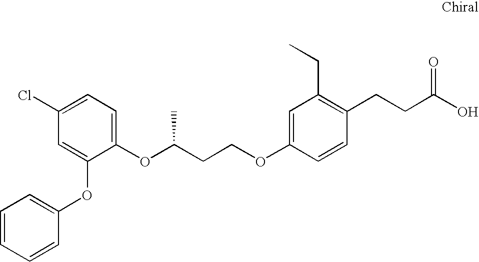 | (R)-3-{4-[3-(4-Chloro-2- phenoxy-phenoxy)- butoxy]-2-ethyl-phenyl}- propionic acid |
| 180 | 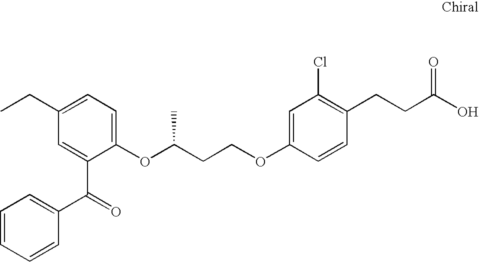 | (R)-3-{4-[3-(2-Benzoyl- 4-ethyl-phenoxy)- butoxy]-2-chloro- phenyl}-propionic acid |
| 181 | 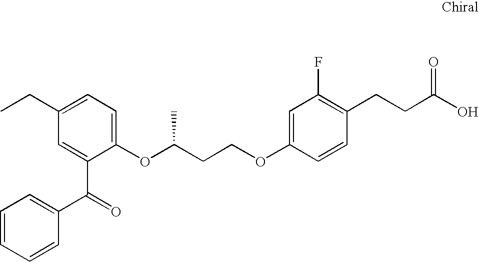 | (R)-3-{4-[3-(2-Benzoyl- 4-ethyl-phenoxy)- butoxy]-2-fluoro- phenyl}-propionic acid |
| 182 | 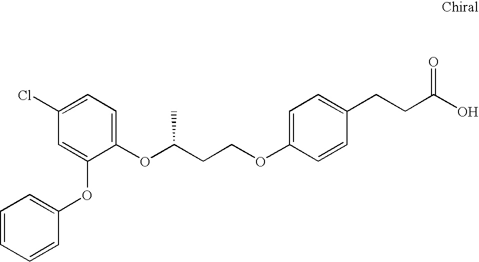 | (R)-3-{4-[3-(4-Chloro-2- phenoxy-phenoxy)- butoxy]-phenyl}- propionic acid |
| 183 | 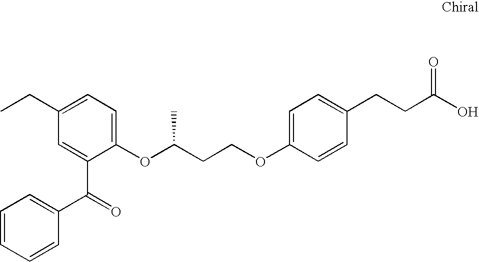 | (R)-3-{4-[3-(2-Benzoyl- 4-ethyl-phenoxy)- butoxy]-phenyl}- propionic acid |
| 184 | 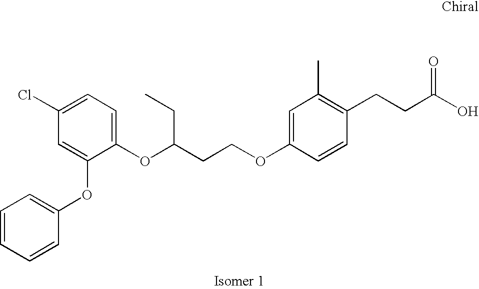 | (R)-3-{4-[3-(4-Chloro-2- phenoxy-phenoxy)- pentyloxy]-2-methyl- phenyl}-propionic acid |
| 185 | 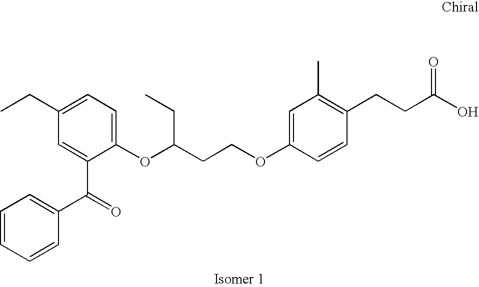 | (R)-3-{4-[3-(2-Benzoyl- 4-ethyl-phenoxy)- pentyloxy]-2-methyl- phenyl}-propionic acid |
| 186 | 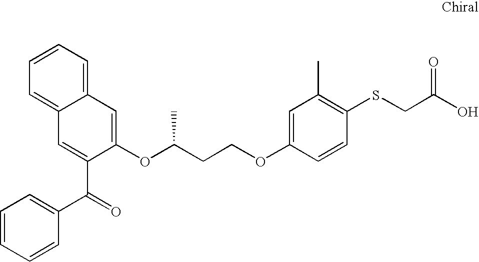 | (R)-{4-[3-(3-Benzoyl- naphthalen-2-yloxy)- butoxy]-2-methyl- phenylsulfanyl}-acetic acid |
| 187 | 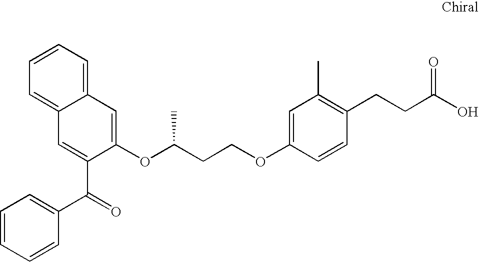 | (R)-3-{4-[3-(3-Benzoyl- naphthalen-2-yloxy)- butoxy]-2-methyl- phenyl}-propionic acid |
| 188 | 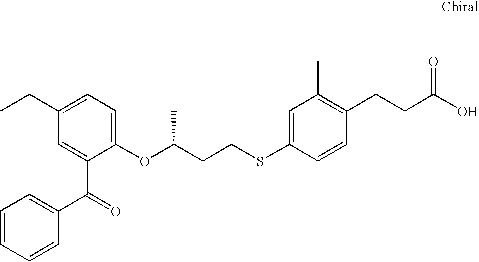 | (R)-3-{4-[3-(4-Ethyl-2- phenoxy-phenoxy)- butylsulfanyl]-2-methyl- phenyl}-propionic acid |
| 189 | 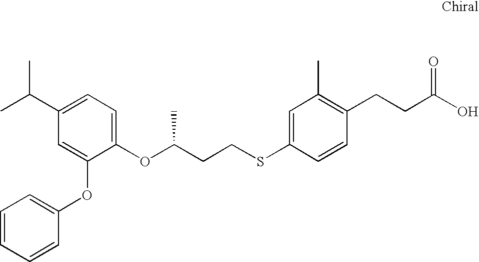 | (R)-3-{4-[3-(4-Isopropyl- 2-phenoxy-phenoxy)- butylsulfanyl]-2-methyl- phenyl}-propionic acid |
| 190 | 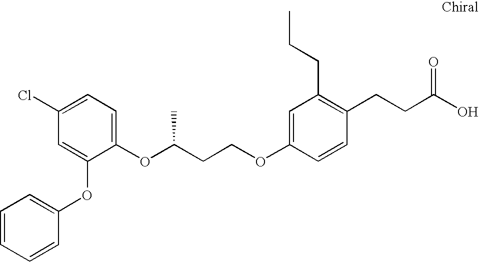 | (R)-3-{4-[3-(4-Chloro-2- phenoxy-phenoxy)- butoxy]-2-propyl- phenyl}-propionic acid |
| 191 | 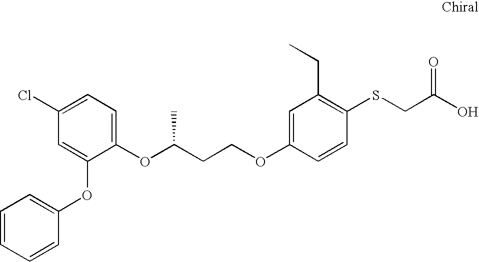 | (R)-{4-[3-(4-Chloro-2- phenoxy-phenoxy)- butoxy]-2-ethyl- phenylsulfanyl}-acetic acid |
| 192 | 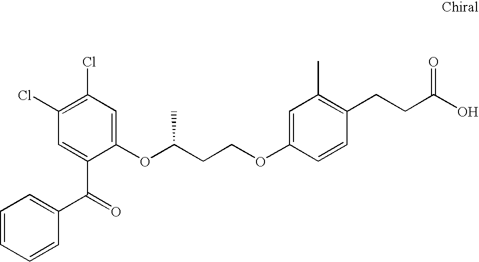 | (R)-3-{4-[3-(2-Benzoyl- 4,5-dichloro-phenoxy)- butoxy]-2-methyl- phenyl}-propionic acid |
| 193 | 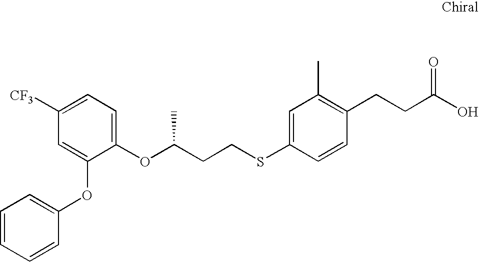 | (R)-3-{2-Methyl-4-[3-(2- phenoxy-4- trifluoromethyl- phenoxy)-butylsulfanyl]- phenyl}-propionic acid |
| 194 | 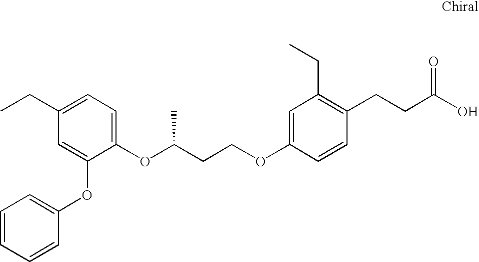 | (R)-3-{2-Ethyl-4-[3-(4- ethyl-2-phenoxy- phenoxy)-butoxy]- phenyl}-propionic acid |
| 195 | 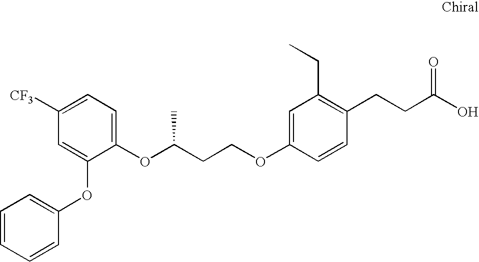 | (R)-3-{2-Ethyl-4-[3-(2- phenoxy-4- trifluoromethyl- phenoxy)-butoxy]- phenyl}-propionic acid |
| 196 | 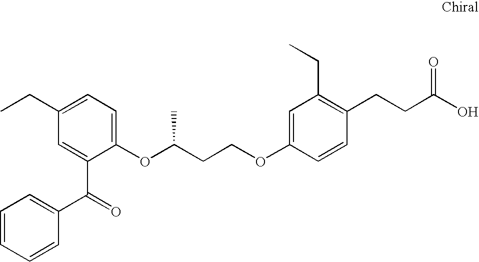 | (R)-3-{4-[3-(2-Benzoyl- 4-ethyl-phenoxy)- butoxy]-2-ethyl-phenyl}- propionic acid |
| 197 | 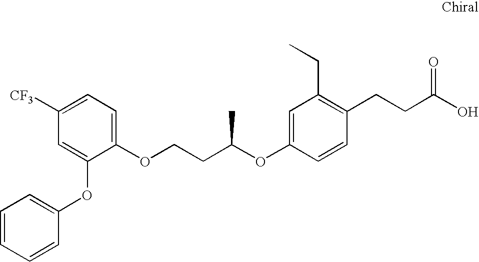 | (R)-3-{2-Ethyl-4-[1- methyl-3-(2-phenoxy-4- trifluoromethyl- phenoxy)-propoxy]- phenyl}-propionic acid |
| 198 | 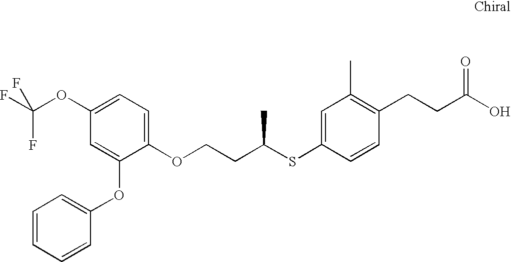 | (R)-3-{2-Methyl-4-[1- methyl-3-(2-phenoxy-4- trifluoromethoxy- phenoxy)- propylsulfanyl]-phenyl}- propionic acid |
| 199 | 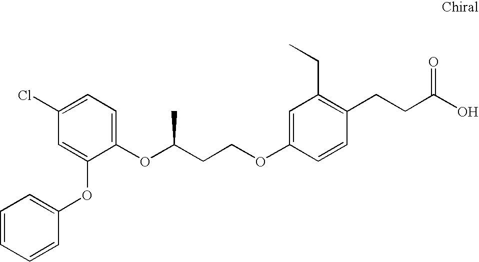 | (S)-3-{4-[3-(4-Chloro-2- phenoxy-phenoxy)- butoxy]-2-ethyl-phenyl}- propionic acid |
| 200 | 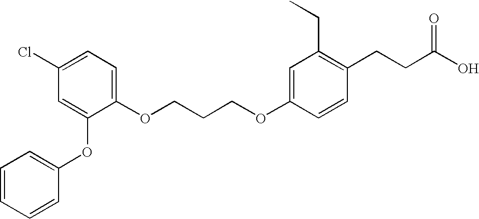 | 3-{4-[3-(4-Chloro-2- phenoxy-phenoxy)- propoxy]-2-ethyl- phenyl}-propionic acid |
| 201 | 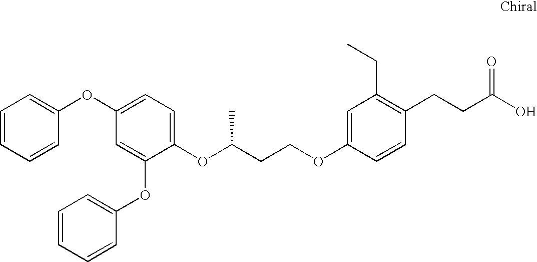 | (R)-3-{4-[3-(2,4- Diphenoxy-phenoxy)- butoxy]-2-ethyl-phenyl}- propionic acid |
| 202 | 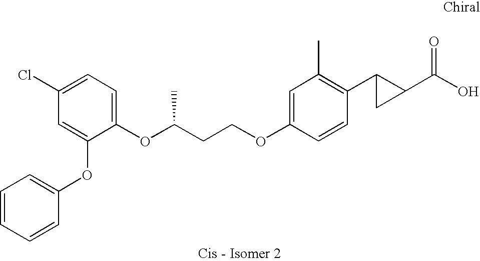 | 2-{4-[4-(4-Chloro-2- phenoxy-phenyl)-3- methyl-butoxy]-2- methyl-phenyl}- cyclopropanecarboxylic acid |
| 203 | 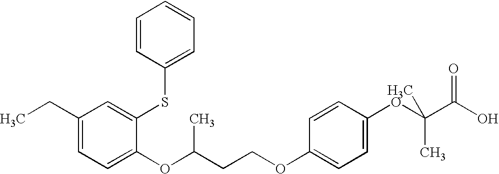 | (R, S)-2-{4-[3-(4-Ethyl- 2-phenylsulfanyl- phenoxy)-butoxy]- phenoxy}-2-methyl- propionic acid |
| 204 | 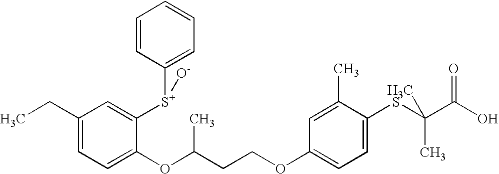 | 2-{4-[3-(R,S-2- Benzenesulfinyl-4-ethyl- phenoxy)-butoxy]- 2-methyl- phenylsulfanyl}-2- methyl-propionic acid (enamtiomer pair 1) |
| 205 | 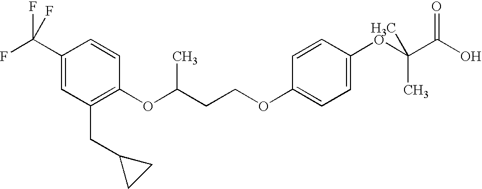 | (R, S)-2-{4-[3-(2- Cyclopropylmethyl-4- trifluoromethyl- phenoxy)-butoxy]- phenoxy}-2-methyl- propionic acid |
| 206 | 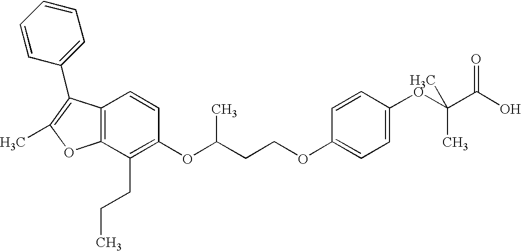 | (R, S)-2-Methyl-2-{4-[3- (2-methyl-3-phenyl-7- propyl- benzofuran-6-yloxy)- butoxy]-phenoxy}- propionic acid |
| 207 | 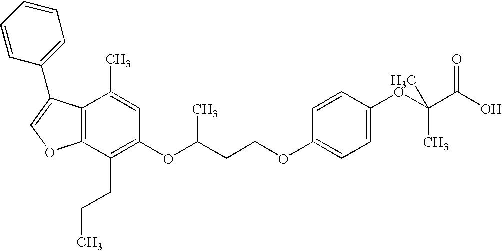 | (R, S)-2-Methyl-2-{4-[3- (4-methyl-3-phenyl-7- propyl- benzofuran-6-yloxy)- butoxyl-phenoxy}- propionic acid |
| 208 | 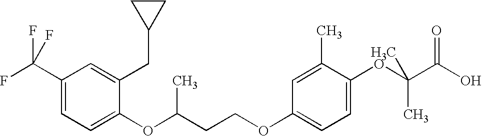 | (R, S)-2-{4-[3-(2- Cyclopropylmethyl-4- trifluoromethyl- phenoxy)-butoxy]-2- methyl-phenoxy}-2- methyl-propionic acid |
| 209 | 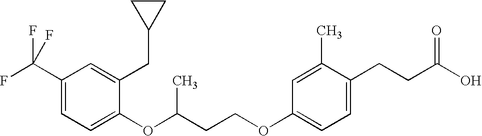 | (R, S)-3-{4-[3-(2- Cyclopropylmethyl-4- trifluoromethyl- phenoxy)-butoxy]-2- methyl-phenyl}- propionic acid |
| 210 | 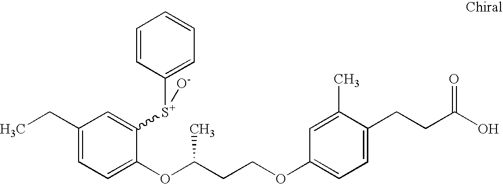 | 3-{R-4-[3-(R, S-2- Benzenesulfinyl-4-ethyl- phenoxy)-butoxy]- 2-methyl-phenyl}- propionic acid |
| 211 | 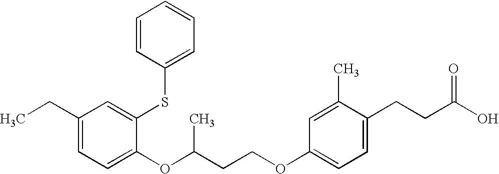 | 3-{4-[3-(4-Ethyl-2- phenylsulfanyl- phenoxy)-butoxy]- 2-methyl-phenyl}- propionic acid isomer 2 |
| 212 | 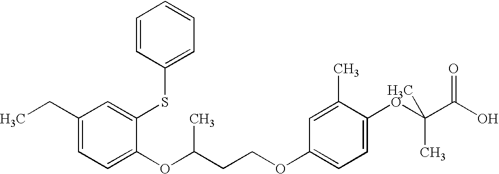 | (R, S)-2-{4-[3-(4-Ethyl- 2-phenylsulfanyl- phenoxy)- butoxy]-phenoxy}-2- methyl-propionic acid |
| 213 | 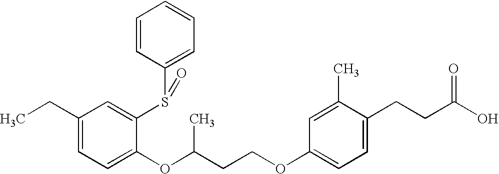 | (R, S)-3-{4-[3-(R, S-2- Benzenesulfinyl-4-ethyl- phenoxy)- butoxy]-2-methyl- phenyl}-propionic acid |
| 214 | 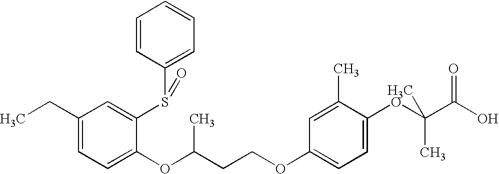 | (R, S)-2-{4-[3-(R, S-2- Benzenesulfinyl-4-ethyl- phenoxy)- butoxy]-2-methyl- phenoxy}-2-methyl- propionic acid |
| 215 | 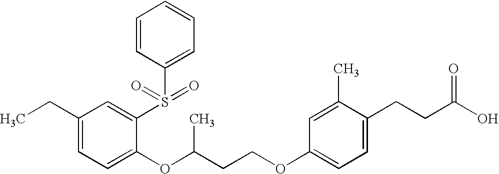 | (R, S)-3-{4-[3-(2- Benzenesulfonyl-4-ethyl- phenoxy)-butoxy]-2- methyl-phenyl}- propionic acid |
| 216 | 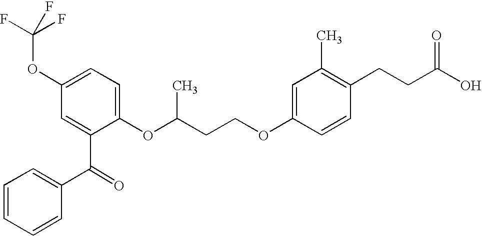 | 3-{4-[3-(2-Benzoyl-4- trifluoromethoxy- phenoxy)-butoxy]-2- methyl-phenyl}- propionic acid |

30. The compound of claim 29, wherein the compound is 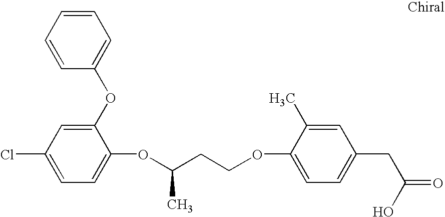
or a pharmaceutically acceptable salt, solvate or hydrate thereof.

31. A pharmaceutical composition comprising a pharmaceutically acceptable carrier and a compound of claim 1 or a pharmaceutically acceptable salt, solvate or hydrate thereof.

32. A pharmaceutical composition comprising:
(1) a compound of claim 1, or a pharmaceutically acceptable salt, solvate, hydrate or stereoisomer thereof;
(2) a second therapeutic agent selected from the group consisting of: insulin sensitizers, sulfonylureas, biguanides, meglitinides, thiazolidinediones, α-glucosidase inhibitors, insulin secretogogues, insulin, antihyperlipidemic agents, plasma HDL-raising agents, HMG-CoA reductase inhibitors, statins, acryl CoA:cholestrol acyltransferase inhibitors, antiobesity compounds, antihypercholesterolemic agents, fibrates, vitamins and aspirin; and
(3) optionally a pharmaceutically acceptable carrier.

33. (canceled)

34. (canceled)

35. (canceled)

36. (canceled)

37. (canceled)

38. (canceled)

39. (canceled)

40. (canceled)

41. (canceled)

42. (canceled)

43. A method for lowering blood-glucose in a mammal in need thereof comprising the step of administering an effective amount of a compound of claim 1.

44. A method of treating or preventing disease or condition in a mammal in need thereof selected from the group consisting of hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component, comprising the step of administering an effective amount of a compound of claim 1.

45. A method of treating or preventing diabetes mellitus in a mammal in need there of comprising the step of administering to a mammal a therapeutically effective amount of a compound of claim 1.

46. A method of treating or preventing cardiovascular disease in a mammal in need thereof comprising the step of administering to a mammal a therapeutically effective amount of a compound of claim 1, or a pharmaceutically acceptable salt, solvate, hydrate or stereoisomer thereof.

47. (canceled)

48. A method of treating or preventing disease or condition in a mammal in need thereof selected from the group consisting of hyperglycemia, dyslipidemia, Type II diabetes, Type I diabetes, hypertriglyceridemia, syndrome X, insulin resistance, heart failure, diabetic dyslipidemia, hyperlipidemia, hypercholesteremia, hypertension, obesity, anorexia bulimia, anorexia nervosa, cardiovascular disease and other diseases where insulin resistance is a component, comprising the step of administering an effective amount of a compound of claim 1 and an effective amount of second therapeutic agent selected from the group consisting of: insulin sensitizers, sulfonylureas, biguanides, meglitinides, thiazolidinediones, α-glucosidase inhibitors, insulin secretogogues, insulin, antihyperlipidemic agents, plasma HDL-raising agents, HMG-CoA reductase inhibitors, statins, acryl CoA:cholestrol acyltransferase inhibitors, antiobesity compounds, antihypercholesterolemic agents, fibrates, vitamins, and aspirin.

49. (canceled)
